(19) United States
(12) Patent Application Publication
(10) Pub. No.: US 2025/0154138 A1
(43) Pub. Date: May 15, 2025

(54) Small Molecule IL-17A Modulators

(71) Applicant: BeiGene, Ltd., Camana Bay (KY)

(72) Inventors: Hai Xue, Beijing (CN); Yunhang Guo, Beijing (CN); Si Chen, Beijing (CN); Zhiwei Wang, Beijing (CN)

(73) Assignee: BeiGene, Ltd., Camana Bay (KY)

(21) Appl. No.: 18/918,712

(22) Filed: Oct. 17, 2024

Related U.S. Application Data
(63) Continuation of application No. PCT/CN2023/089491, filed on Apr. 20, 2023.

(30) Foreign Application Priority Data
Apr. 21, 2022 (WO) PCT/CN22/88080

(51) Int. Cl. C07D 409/14; A61K 31/4178; A61K 31/4245; A61K 31/527; C07D 403/12; C07D 405/14; C07D 413/12; C07D 413/14

(52) U.S. Cl. CPC C07D 409/14; A61K 31/4178; A61K 31/4245; A61K 31/527; C07D 403/12; C07D 405/14; C07D 413/12; C07D 413/14

(57) ABSTRACT

Disclosed herein are fused bi-cyclic compounds used as Small Molecule IL-17A Modulators. Disclosed herein is the use of these Small Molecule IL-17A Modulators for the use of decreasing IL-17 activity by inhibition, and the use of such compounds for the use in the treatment of autoimmune diseases or inflammatory diseases.

### CROSS-REFERENCE TO RELATED APPLICATIONS

This application is a continuation of International Application No. PCT/CN2023/089491, filed Apr. 20, 2023, which claims priority to International Application No. PCT/CN2022/088080, filed Apr. 21, 2022. The disclosures of the aforementioned applications are incorporated herein by reference in their entireties.

### FIELD OF THE INVENTION

Disclosed herein are fused bi-cyclic compounds used as Small Molecule IL-17A Modulators. Disclosed herein is the use of these Small Molecule IL-17A Modulators for the use of decreasing IL-17 activity by inhibition, and the use of such compounds for the use in the treatment of autoimmune diseases or inflammatory diseases.

### BACKGROUND OF THE INVENTION

Interleukin-17(IL-17) family is a group of pro-inflammatory cytokines, which are involved in the immune response of tissues and played a critical role in chronic inflammation. The secretion of IL-17 can stimulate the production of other proinflammatory cytokines (IL-1, IL-6, G-CSF, GM-CSF, and TNF) and chemokines (CXCL1, CXCL2, CXCL5, CCL2, CCL7, CCL20, and IL-8), matrix metalloproteinases (MMP1, MMP3, MMP9, and MMP13), and anti-microbial peptides (β-defensins, S-100 proteins) (Frontiers in Immunology (2020) 11: 947). IL-17 family is composed of 7 members including IL17A to IL17F and vIL-17A. IL-17A, as the founder member with most widely studied, is associated with host defense against various microbial pathogens and tissue inflammation (Gene (2017) 614: 8-14). IL-17A consists of 155 amino acids and the molecular mass of the homodimer is 30-35 kDa (European Respiratory Journal (2005): 159-172). IL-17F, the closest relative of IL-17A, shares around 50% amino acid sequence homology with IL-17A over its 163 amino acids and is often co-expression with IL-17A (Immunity (2004): 467-476). Both IL-17A and IL-17F are secreted from T helper cells (Th17) and expressed as either homodimers or IL-17A/F heterodimer (European Respiratory Journal (2005): 159-172). The family of IL-17 receptors composes of five members: IL-17RA, IL-17RB, IL-17RC, IL-17RD, and IL-17RE. All of these five receptors share a common cytoplasmic motif named as SEFIR domain (Frontiers in Immunology 11 (2020): 947). The signal pathway of IL-17A and IL-17F through the same receptor complex IL-17R consists of two subunits: IL-17RA and IL-17RG (Gene (2017) 614: 8-14). The correspondent receptors activate the downstream pathway of these signaling including NFκB, MAPKs and C/EBPs to induce the expression of anti-microbial peptides, cytokines and chemokines (Frontiers in Immunology (2020): 947). IL-17A promotes tissue inflammation and bone remodeling. It can act on various cell types: keratinocytes, endothelial cells, fibroblasts, osteoclasts, chondrocytes, and osteoblasts (Archives of oral biology (2014): 897-905).

The abnormal autoimmune response of IL-17A increasing secretion is observed in many autoimmune diseases including psoriasis, spondylarthritis, rheumatoid arthritis, and multiple sclerosis (Gene (2017) 614: 8-14). One potential treatment of these diseases is to develop IL-17A inhibitors. Currently, several monoclonal antibodies targeting IL-17A are approved by the FDA for the treatment of moderate-to-severe plaque psoriasis (Expert Opin. Biol. Ther. (2019), 19, 45-54): secukinumab (Cosentyx, Novartis), ixekizumab (Taltz, Eli Lilly), and brodalumab (Kyntheum, LEO Pharma/AstraZeneca). Bimekizumab (UCB) is a bispecific anti-IL-17A/IL-17F humanized monoclonal antibody that was just approved in 2021 by EC. However, since monoclonal antibodies have a number of disadvantages, such as high cost-of-goods, non-oral applications, poor tissue penetration, and often long half-lives (Chem. Biol. (2014) 21, 1102-1114), the search for small molecules that would exhibit the same biological outcome is still ongoing. Some disclosures are known to describe the small molecule with IL-17A inhibition activity (WO2014066726, WO2018229079, WO2019138017, WO2019223718, WO2020182666, WO2020011731, WO2020127685, WO2020163554, WO2021055376, WO2021098844, 2020163554 WO2020146194, WO2021204801, WO2021170627, WO2021098844, WO2021222404, WO2021220183). Several small molecule inhibitors targeting protein/protein interaction of IL-17A and IL-17RA have been advanced into clinical trials now. Oral administration and flexible treatment regimen are supposed to be main aspects in favor of patient's convenience. The possibility of fast withdraw of small molecule drug may present approved safety when target-related adverse events occur. Therefore, there is a continuous need to develop small molecule IL-17A modulator with diverse structure, particularly that can be orally administrated.

### SUMMARY OF THE INVENTION

In one embodiment, disclosed herein is fused bi-cyclic compound of Formula (I). The embodiment comprises the following aspects:

Aspect 1. A compound of Formula (I):

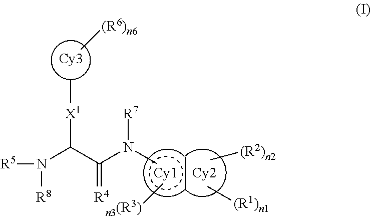

or a N-oxide thereof, or a pharmaceutically acceptable salt thereof, or a stereoisomer thereof, or a deuterated analog thereof, wherein:
Cy1 is a 5-6 membered aromatic ring, said ring comprising 0-3 heteroatoms independently selected from nitrogen, oxygen or sulfur; said ring is optionally substituted with at least one substituent R³;
n3 is independently 0, 1, 2 or 3;
Cy2 is a 4-8 membered saturated or partially or completely unsaturated ring, said ring comprising 0-3 heteroatoms independently selected from nitrogen, oxygen or optionally oxidized sulfur; said ring is substituted with at least one substituent R¹ and/or R²;
n1 is 0 or 1;
n2 is 0, 1 or 2;
n1 and n2 are not 0 at the same time;
Cy1 and Cy2 are fused with each other;
Cy3 is a 3-12 membered saturated or partially or completely unsaturated ring, said ring comprising 0-3 heteroatoms independently selected from nitrogen, oxygen or optionally oxidized sulfur; said ring is optionally substituted with at least one substituent R⁶;
n6 is independently 0, 1, 2, 3, 4 or 5, provided that the valency theory is met;
X¹ is a single bond or —CR⁹R¹⁰—;
R¹ is independently selected from H or

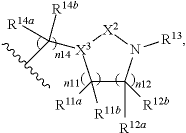

wherein X² is N or C(R¹⁵);
   X³ is —C(O)—, —S(O)— or —S(O)₂—;
   n11 and n12 are each independently 1, 2 or 3;
   n14 is 0 or 1;
R¹⁵ is selected from hydrogen, halogen, —C₁₋₈alkyl, —C₃-C₈cycloalkyl, 3- to 8-membered heterocyclyl, —CN, —OR^{15a}, —NR^{15a}R^{15b}, or —NR^{15a}COR^{15b}; each of —C₁₋₈alkyl, C₃-C₈cycloalkyl and 3- to 8-membered heterocyclyl is optionally substituted with at least one substituent R^{15c},
R^{15a} and R^{15b} are each independently selected from hydrogen, —C₁₋₈alkyl, —C₁₋₈haloalkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of —C₁₋₈alkyl, —C₁₋₈haloalkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl and 5- to 12-membered heteroaryl is optionally substituted with at least one substituent R^{15d};
R^{15c} and R^{15d}, at each occurrence, are each independently halogen, —OH, —CN, oxo, —C₁₋₈alkyl, —C₁₋₈ haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
R^{11a}, R^{11b}, R^{12a} and R^{12b} are each independently selected from hydrogen, halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, —CN, —OR^{11c}, —COR^{11c}, —CO₂R^{11c}, —CONR^{11c}R^{11d}, —NR^{11c}R^{11d} or —NR^{11c}COR^{11d}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{11e}, or
(R^{11a} and R^{11b}), (R^{12a} and R^{12b}), (R^{11a} and R^{12a}), (R^{11a} and R^{12b}), (R^{11b} and R^{12a}) or (R^{11b} and R^{12b}) together with the atom(s) to which they are attached, form a 3- to 8-membered unsaturated or saturated ring, said ring comprising 0-3 heteroatoms independently selected from nitrogen, oxygen or sulfur; said ring is optionally substituted with at least one substituent R^{11f};
R^{11e} and R^{11f} at each occurrence, are each independently hydrogen, halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo (═O), —OR^{11g}, thioxo (═S), —SR^{11g}, —CN, —SO₂R^{11g}, —SO₂NR^{11g}R^{11h}, —COR^{11g}, —CO₂R^{11h}, —CONR^{11g}R^{11h}, —NR^{11g}R^{11h}, —NR^{11g}COR^{11h}, —NR^{11g}CO₂R^{11h} or —NR¹SO₂R^{11h}; each of —C₁₋₈alkyl, —C₁₋₈alkoxy, C₃-C₈cycloalkyl, 3- to 8-membered heterocyclyl, C₆-C₁₂aryl or 5- to 12-membered heteroaryl is optionally substituted with at least one substituent R¹¹ⁱ;
R^{11c}, R^{11d}, R^{11g} and R^{11h} are each independently selected from hydrogen, —C₁₋₈alkyl, —C₁₋₈haloalkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl; each of —C₁₋₈alkyl, —C₁₋₈haloalkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl and 5- to 12-membered heteroaryl is optionally substituted with at least one substituent R^{11j};
at each occurrence, R¹¹ⁱ and R^{11j} are each independently halogen, —OH, —CN, oxo, —C₁₋₈alkyl, —C₁₋₈ haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl; each of —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl is optionally substituted with at least one substituent halogen, —OH, —CN, oxo, —C₁₋₈alkyl, —C₁₋₈haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
R¹³, R^{14a} and R^{14b} are each independently selected from hydrogen, —C₁₋₈alkyl, —C₃-C₈cycloalkyl or 3- to 8-membered heterocyclyl; each of —C₁₋₈alkyl, —C₃-C₈cycloalkyl and 3- to 8-membered heterocyclyl is optionally substituted with at least one substituent R^{13a};
R^{13a}, at each occurrence, is independently halogen, —OH, —CN, oxo, —C₁₋₈alkyl, —C₁₋₈haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
R² is independently selected from H, —C₁₋₈alkyl, —C₂₋₈alkenyl, —C₂₋₈alkynyl, —C₃-C₈cycloalkyl, C₆-C₁₂aryl, 5- to 12-membered heteroaryl, —CN, —COR^{2a}, —CO₂R^{2a}, —CONR^{2a}R^{2b}, —NR^{2a}R^{2b}, or —NR^{2a}COR^{2b}, each of —C₁₋₈alkyl, —C₂₋₈alkenyl, —C₂₋₈alkynyl, —C₃-C₈cycloalkyl, C₆-C₁₂aryl and 5- to 12-membered heteroaryl is optionally substituted with at least one substituent R^{2c}; or
when geminal or adjacent, two R² together with the carbon atom(s) to which they are attached, form a 3- to 8-membered unsaturated or saturated ring, said ring comprising 0-3 heteroatoms independently selected from nitrogen, oxygen or sulfur; said ring is optionally substituted with at least one substituent R^{2c};
R^{2a} and R^{2b} are each independently selected from hydrogen, —C₁₋₈alkyl, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{2d}; or
R^{2a} and R^{2b} together with the nitrogen atom to which they are attached, form a 3- to 8-membered unsaturated or saturated ring, said ring comprising 0-3 heteroatoms independently selected from nitrogen, oxygen or sulfur; said ring is optionally substituted with at least one substituent R^{2d};
R^{2c} and R^{2d}, at each occurrence, are each independently hydrogen, halogen, —C₁₋₈alkyl, —C₃-C₈cycloalkyl, 3- to 8-membered heterocyclyl, C₆-C₁₂aryl, 5- to 12-membered heteroaryl, oxo (═O), —OR², —SR^{2e}, —CN, —COR^{2e}, —CO₂R^{2e}, —CONR^{2e}R^{2f}, —NR^{2e}R^{2f}, —NR^{2e}COR^{2f} or —NR^{2e}CO₂R^{2f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{2g};
R^{2e} and R^{2f}are each independently selected from hydrogen, —C₁₋₈alkyl, —C₁₋₈alkoxy, C₁₋₈alkoxy-C₁₋₈ alkyl-, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₁₋₈alkoxy, C₁₋₈alkoxy-C₁₋₈alkyl-, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{2h};
R^{2g} and R^{2h}, at each occurrence, are each independently halogen, —OH, —C₁₋₈alkyl, —C₁₋₈haloalkyl, —C₁₋₈alkoxy, C₁₋₈alkoxy-C₁₋₈alkyl-, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
when n1 and n2 are both 1, R¹ and R² are geminal or adjacent;
R³ is independently selected from hydrogen, halogen, —C₁₋₈alkyl, —C₁₋₈alkoxy, —C₃-C₈cycloalkyl, or —CN, wherein each of —C₁₋₈alkyl, —C₁₋₈alkoxy and —C₃-C₈cycloalkyl is optionally substituted with at least one substituent selected from halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁴ is oxo (═O) or thioxo (═S); or
R³ and R⁴ together with the atoms to which they are attached, form a 5- to 6-membered aromatic ring, said ring comprising 1-3 heteroatoms independently selected from nitrogen, oxygen or sulfur; said ring is optionally substituted with at least one substituent R^{3a};
R^{3a} is independently selected from hydrogen, halogen, —C₁₋₈alkyl, —C₁₋₈alkoxy, —C₃-C₈cycloalkyl, or —CN, wherein each of —C₁₋₈alkyl, —C₁₋₈alkoxy and —C₃-C₈cycloalkyl is optionally substituted with at least one substituent selected from halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁵ is C₆-C₁₂aryl, 5- to 12-membered heteroaryl, —COR^{5a}, —CO₂R^{5a}, —CONR^{5a}R^{5b}, wherein each of C₆-C₁₂aryl and 5- to 12-membered heteroaryl is optionally substituted with at least one substituent R^{5c};
R^{5a} and R^{5b} are each independently selected from —C₁₋₈alkyl, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{5d}; or
R^{5a} and R^{5b} together with the atom to which they are attached, form a 3- to 8-membered unsaturated or saturated ring, said ring comprising 0-3 heteroatoms independently selected from nitrogen, oxygen or sulfur; said ring is optionally substituted with at least one substituent R^{5d};
R^{5c} and R^{5d}, at each occurrence, are each independently selected from hydrogen, halogen, —C₁₋₈alkyl, —C₃-C₈cycloalkyl, 3- to 8-membered heterocyclyl, C₆-C₁₂aryl, 5- to 12-membered heteroaryl, oxo (═O), —OR^{5e}, —SR^{5e}, —CN, —COR^{5e}, —CO₂R^{5e}, —CONR^{5e}R^{5f}, —NR^{5e}R^{5f}, —NR^{5e}COR^{5f} or —NR^{5e}CO₂R^{5f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{5g};
R^{5e} and R^{5f}are each independently selected from hydrogen, —C₁₋₈alkyl, —C₁₋₈alkoxy, C₁₋₈alkoxy-C₁₋₈ alkyl-, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₁₋₈alkoxy, C₁₋₈alkoxy-C₁₋₈alkyl-, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{5h};
R^{5g} and R^{5h} are each independently selected from halogen, —OH, —CN, —C₁₋₈alkyl, —C₁₋₈alkoxy, C₁₋₈ alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁶ is H, halogen, —C₁₋₈alkyl, —C₂₋₈alkenyl, —C₂₋₈alkynyl, —C₃-C₈cycloalkyl, C₆-C₁₂aryl, 5- to 12-membered heteroaryl, —CN, —OR^{6a}, —NR^{6a}R^{6b}, —COR^{6a}, —CO₂R^{6a}, —CONR^{6a}R^{6b}, —COR^{6a} or —NR^{6a}COR^{6b}each of —C₁₋₈alkyl, —C₂₋₈alkenyl, —C₂₋₈alkynyl, —C₃-C₈cycloalkyl, C₆-C₁₂aryl and 5- to 12-membered heteroaryl is optionally substituted with at least one substituent R^{6d};
R^{6a} and R^{6b} are each independently selected from hydrogen, —C₁₋₈alkyl, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{6d};
R^{6e}, at each occurrence, is independently hydrogen, halogen, —C₁₋₈alkyl, —C₃-C₈cycloalkyl, 3- to 8-membered heterocyclyl, C₆-C₁₂aryl, 5- to 12-membered heteroaryl, oxo (═O), —OR^{6e}, —SR^{6e}, —CN, —COR^{6e}, —CO₂R^{6e}, —CONR^{6e}R^{6f}, —NR^{6e}R^{6f}, —NR^{6e}COR^{6f} or —NR^{5e}CO₂R^{5f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{6g};
R^{6e} and R^{6f} are each independently selected from hydrogen, —C₁₋₈alkyl, —C₁₋₈alkoxy, C₁₋₈alkoxy-C₁₋₈ alkyl-, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl;
R^{6d} and R^{6g} are each independently selected from halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁷ and R⁸ are each independently hydrogen;
R⁹ and R¹⁰ are each independently selected from H, halogen, —C₁₋₈alkyl, —C₂₋₈alkenyl, —C₂₋₈alkynyl, —C₃-C₈cycloalkyl, C₆-C₁₂aryl or 5- to 12-membered heteroaryl, each of —C₁₋₈alkyl, —C₂₋₈alkenyl, —C₂₋₈alkynyl, —C₃-C₈cycloalkyl, C₆-C₁₂aryl and 5- to 12-membered heteroaryl is optionally substituted with at least one substituent R^{9a}; and
R^{9a}, at each occurrence, is independently halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl.

Aspect 2. The compound of Aspect 1, wherein the compound is selected from Formula (IIa)-(IIp):

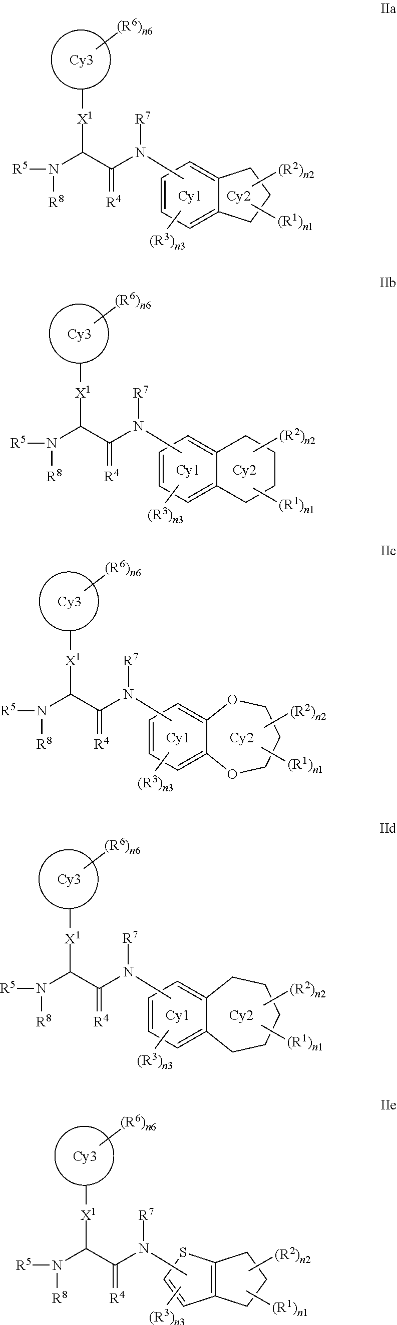

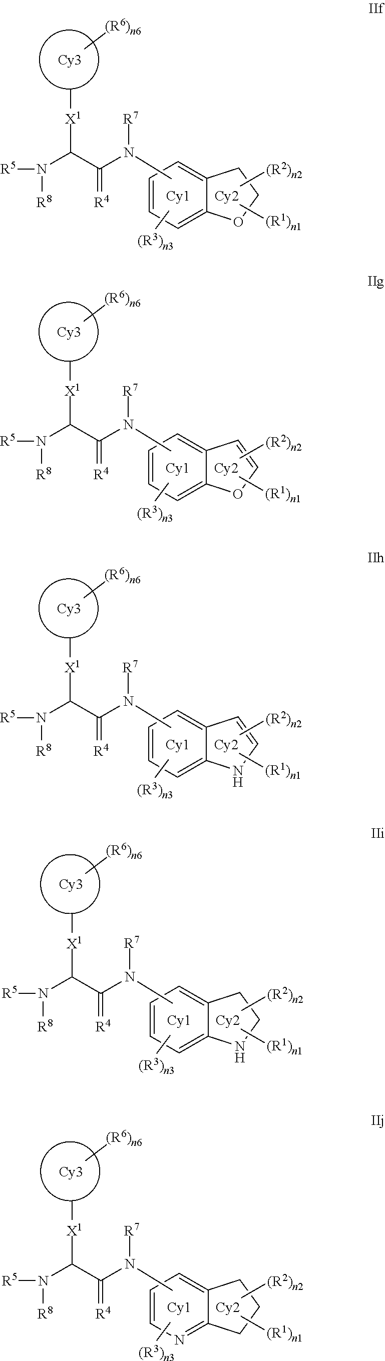

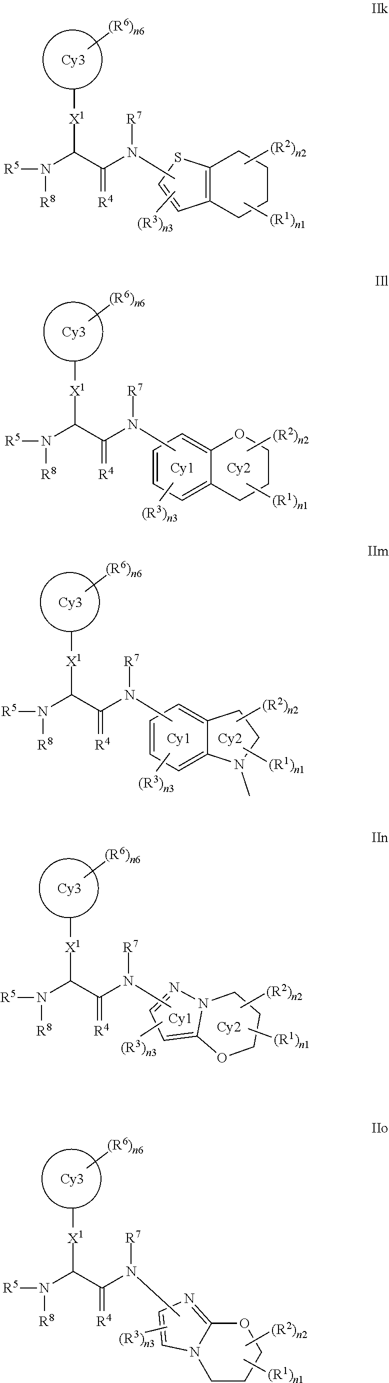

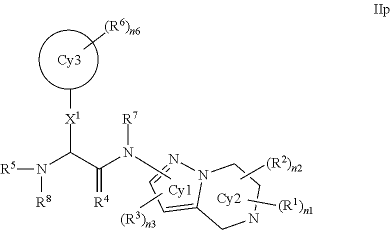

wherein, R¹, R², R³, R⁴, R⁵, R⁶, R⁷, R⁸, Cy3, n1, n2, n13, n6 and X¹ are defined as in Aspect 1.

Aspect 3. The compound of Aspect 1, wherein the compound is selected from Formula

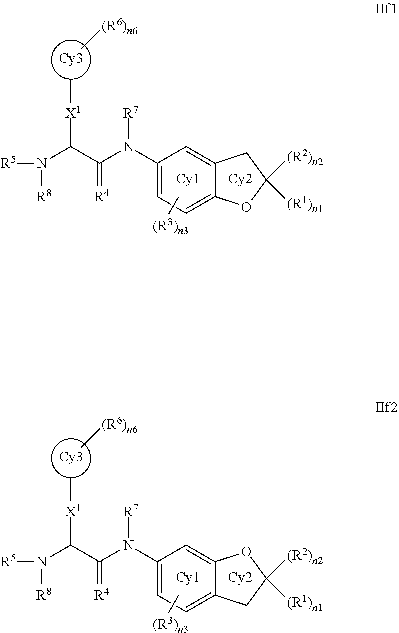

wherein, R¹, R², R³, R⁴, R⁵, R⁶, R⁷, R⁸, Cy3, n1, n2, n3, n6 and X¹ are defined as in Aspect 1.

Aspect 4. The compound of Aspect 1, wherein the compound is selected from Formula (IIIa)-(IIIb):

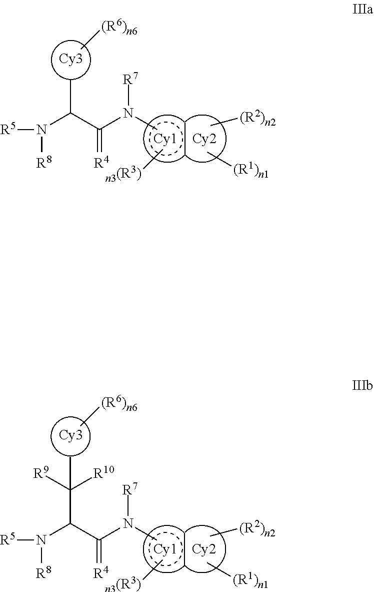

wherein, R¹, R², R³, R⁴, R⁵, R⁶, R⁷, R⁸, Cy1, Cy2, Cy3, n1, n2, n3, n6, R⁹ and R¹⁰ are defined as in Aspect 1.

Aspect 5. The compound of Aspect 1, wherein the compound is selected from Formula (IVa)-(IVb):

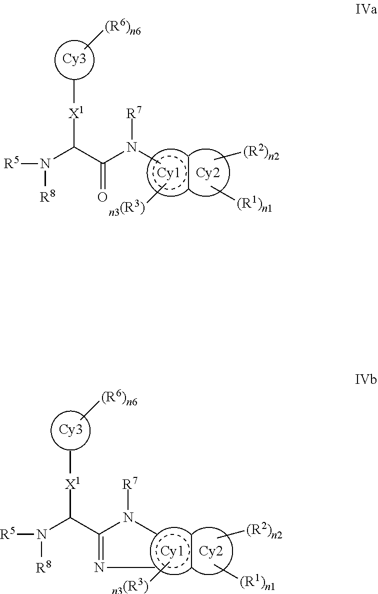

wherein, X¹, R¹, R², R³, R⁵, R⁶, R⁷, R⁸, Cy1, Cy2, Cy3, n1, n2, n3, n6, R⁹ and R¹⁰ are defined as in Aspect 1.

Aspect 6. The compound of Aspect 1, wherein the compound is selected from Formula (Va)-(Vc):

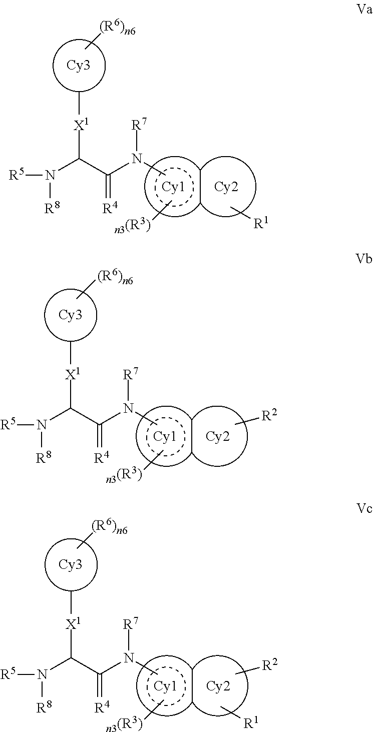

wherein, X¹, R¹, R², R³, R⁴, R⁵, R⁶, R⁷, R⁸, Cy1, Cy2, Cy3, n3, n6, R⁹ and R¹⁰ are defined as in Aspect 1.

Aspect 7. The compound Aspect 1, wherein the compound is VIa,

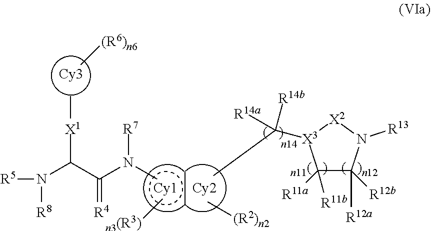

preferably, the compound is selected from VIb and Vic

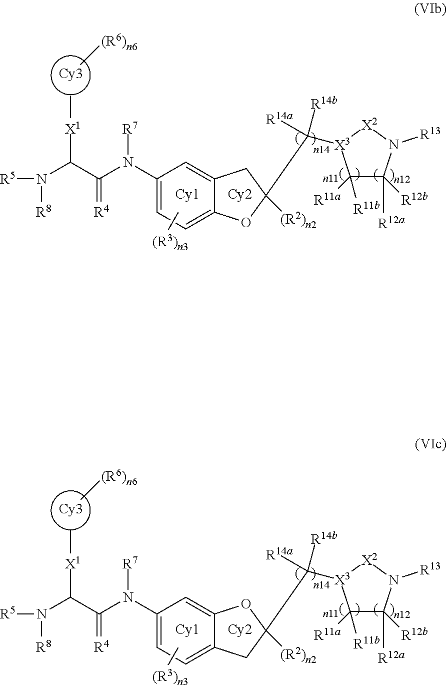

wherein, X¹, X², X³, R², R³, R⁴, R⁵, R⁶, R⁷, R⁸, Cy1, Cy2, Cy3, n2, n3, n6, R^{11a}, R^{11b}, R^{12a}, R^{12b}, R¹³, R^{14a}, R^{14b}, n11, n12 and n14 are defined as in Aspect 1.

Aspect 8. The compound Aspect 1, wherein the compound is VIIa or VIIb,

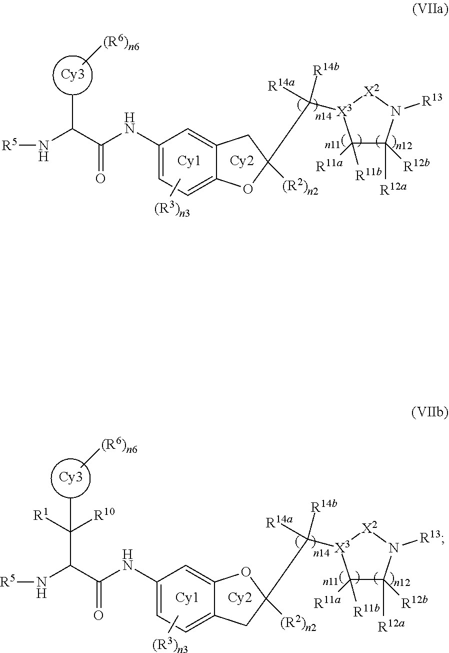

preferably, the compound is selected from VIIc, VIId, VIIe, VIIf, VIIg or VIIh,

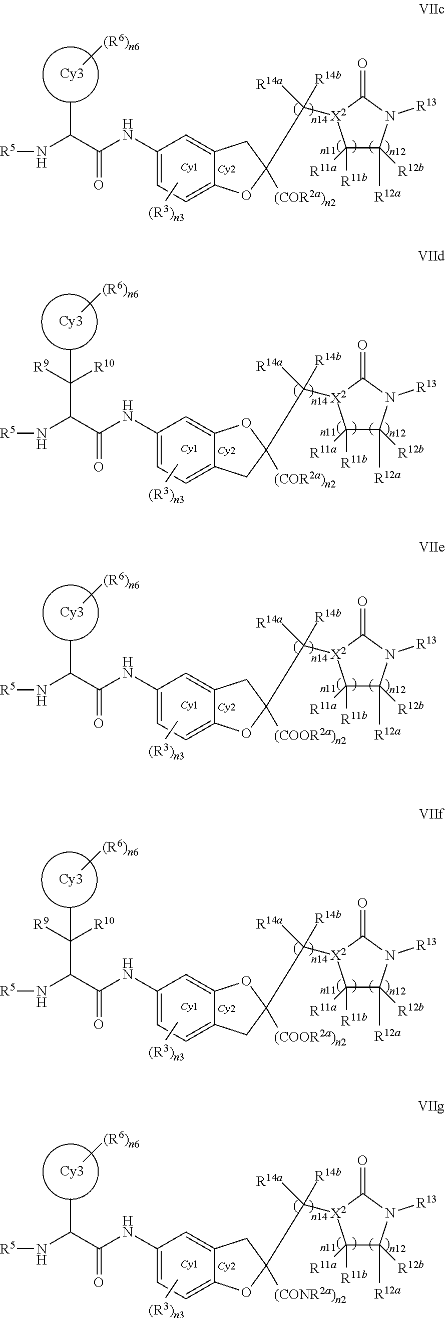

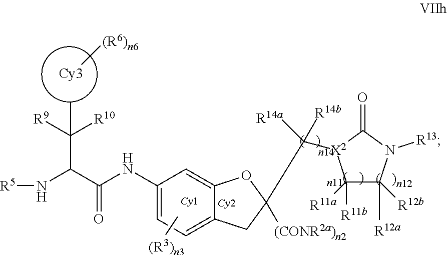

wherein, X², R^{2a}, R³, R⁵, R⁶, R⁹, R¹⁰, Cy1, Cy2, Cy3, n2, n3, n6, R^{11a}, R^{11b}, R^{12a}, R^{12b}, R¹³, R^{14a}, R^{14b}, n11, n12 and n14 are defined as in Aspect 1.

Aspect 9. The compound of any one of the preceding Aspects, wherein Cy1 is a 5- or 6-membered aromatic ring, said ring comprising 0, 1 or 2 heteroatoms independently selected from nitrogen, oxygen or sulfur; said ring is optionally substituted with 0, 1, 2 or 3 substituents R³;
preferably, Cy1 is phenyl, pyridyl, thiophenyl, imidazolyl, furanyl or pyrazolyl; each of phenyl, pyridyl, thiophenyl, imidazolyl, furanyl or pyrazolyl is optionally substituted with 0, 1 or 2 substituents R³.

Aspect 10. The compound of any one of the preceding Aspects, wherein Cy2 a 4-, 5-, 6-, 7- or 8-membered saturated or partially or completely unsaturated ring, said ring comprising 0, 1, 2 or 3 heteroatoms independently selected from nitrogen or oxygen; said ring is substituted with at least one substituent R¹ and/or R²;
preferably, Cy2 a 5-, 6- or 7-membered saturated or partially or completely unsaturated ring, said ring comprising 0, 1 or 2 heteroatoms independently selected from nitrogen or oxygen; said ring is substituted with at least one substituent R¹ and/or R².

Aspect 11. The compound of any one of the preceding Aspects, wherein

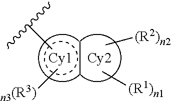

moiety is selected from:

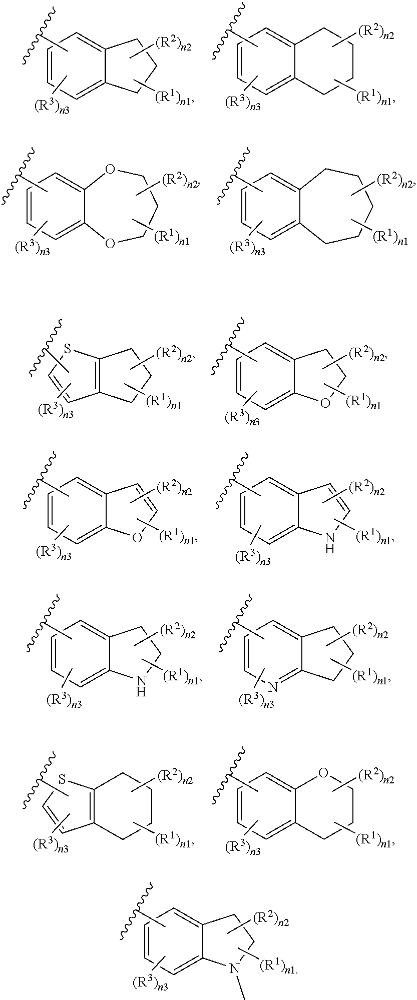

Aspect 12. The compound of any one of the preceding Aspects, wherein Cy3 is a 3-, 4-, 5-, 6-, 7-, 8-, 9-, 10-, 11- or 12-membered saturated or partially or completely unsaturated ring, said ring comprising 0, 1, 2 or 3 heteroatoms independently selected from nitrogen, oxygen or optionally oxidized sulfur; said ring is optionally substituted with 0, 1, 2, 3, 4 or 5 substituent R⁶;
preferably, Cy3 is a 3-, 4-, 5-, 6-, 7-, 8-, 9-, 10-, 11- or 12-membered saturated or partially or completely unsaturated ring, said ring is mono-cycle, bi-cycle or tri-cycle comprising 0, 1, 2 or 3 heteroatoms independently selected from nitrogen, oxygen or optionally oxidized sulfur; said ring is optionally substituted with 0, 1, 2, 3, 4 or 5 substituent R⁶;
more preferably,

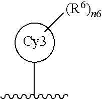

moiety is

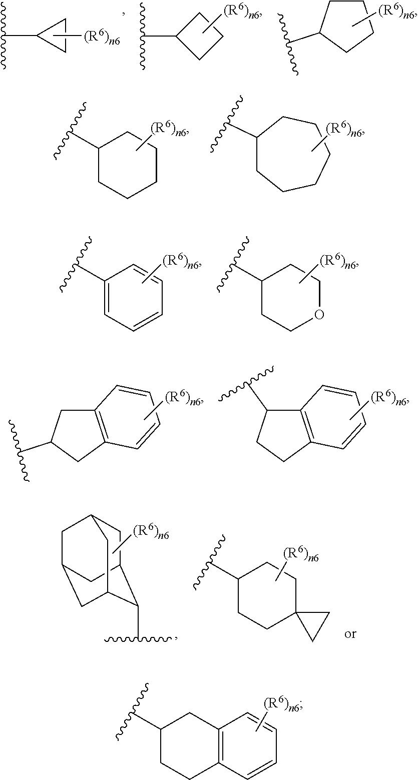

even more preferably,

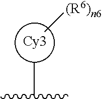

moiety is

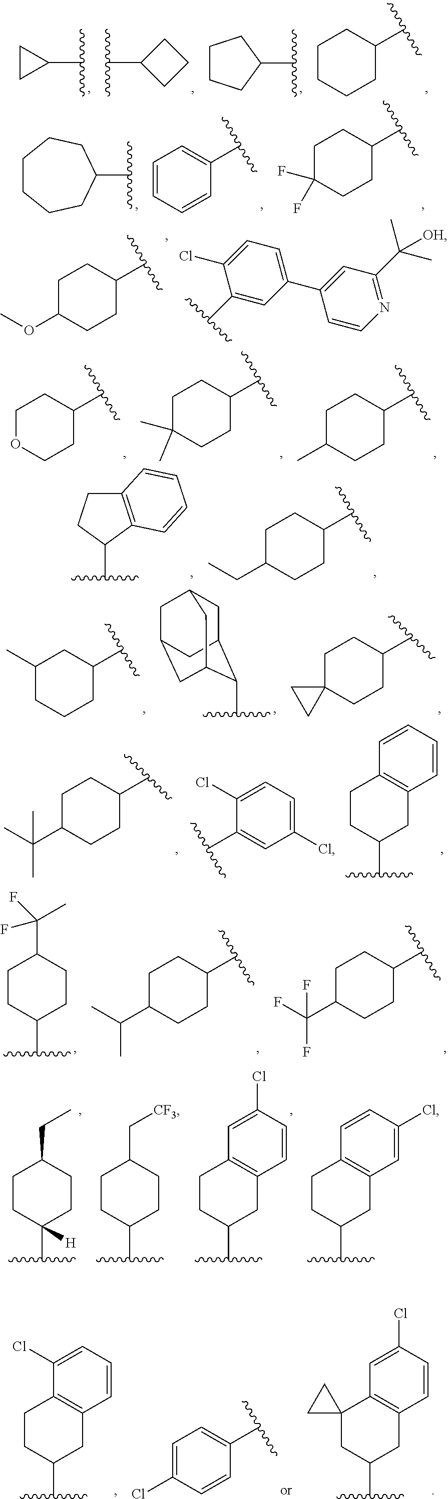

Aspect 13. The compound of any one of the preceding Aspects, wherein R¹ is independently selected from H,

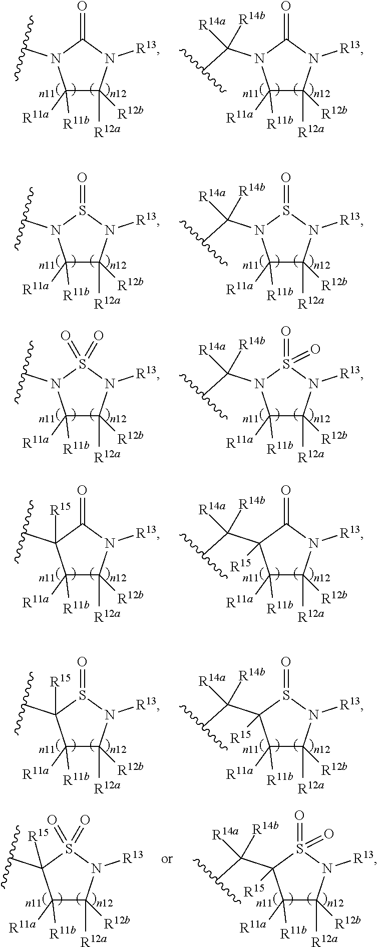

wherein R^{11a}, R^{11b}, R^{12a}, R^{12b}, R¹³, n11 and n12 are defined as in Aspect 1.

Aspect 14. The compound of any one of the preceding Aspects, wherein R¹⁵ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —CN, —OR^{15a}, —NR^{15a}R^{15b}, or —NR^{15a}COR^{15b}; each of methyl, ethyl, propyl, butyl, pentyl, hexyl, heptyl, octyl, cyclopropyl, cyclobutyl, cyclopentyl, cyclohexyl, cycloheptyl, cyclooctyl and 3- to 8-membered heterocyclyl is optionally substituted with at least one substituent R^{15C};
R^{15a} and R^{15b} are each independently selected from hydrogen, methyl, ethyl, propyl, butyl, pentyl, hexyl, heptyl, octyl, —C₁₋₈haloalkyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl, each of methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, C₁₋₈ alkoxy-C₁₋₈alkyl-, cyclopropyl, cyclobutyl, cyclopentyl, cyclohexyl, cycloheptyl, cyclooctyl, 3- to 8-membered heterocyclyl, C₆-C₁₂aryl and 5- to 12-membered heteroaryl is optionally substituted with at least one substituent R^{15d};
R^{15c} and R^{15d}, at each occurrence, are each independently —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, phenyl, or 5- to 12-membered heteroaryl.

Aspect 15. The compound of any one of the preceding Aspects, wherein R¹⁵ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —CN, —OH, methoxy, ethoxy, propoxy, butoxy, pentoxy, hexoxy, heptoxy, octoxy;
preferably, R¹⁵ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl;
more preferably, R¹⁵ is selected from hydrogen, —F, —Cl, —Br, —I, methyl, ethyl, propyl, butyl, pentyl, hexyl, heptyl, octyl;
even more preferably, R¹⁵ is selected from hydrogen.

Aspect 16. The compound of any one of the preceding Aspects, wherein R^{11a}, R^{11b}, R^{12a} and R^{12b} are each independently selected from hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN; each of methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl and pyrazinyl is optionally substituted with at least one substituent R^{11e};
R^{11e}, at each occurrence, is independently hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, oxo (═O), —OR^{11g}, thioxo (═S), —SR^{11g}, —CN, —SO₂R^{11g}, —SO₂NR^{11g}R^{11h}, —COR^{11g}, —CO₂R^{11h}, —CONR^{11g}R^{11h}, —NR^{11g}R^{11h}, —NR^{11g}COR^{11h}, —NR^{11g}CO₂R^{11h} or —NR^{11g}SO₂R^{11h}; each of methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl and pyrazinyl is optionally substituted with at least one substituent R¹;
R^{11c}, R^{11d}, R^{11g} and R^{11h} are each independently selected from hydrogen, methyl, ethyl, propyl, butyl, pentyl, hexyl, heptyl, octyl, —C₁₋₈haloalkyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, —C₁₋₈ haloalkyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one substituent R^{11j};
at each occurrence, R¹¹ⁱ and R^{11j} are each independently —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, —C₃-C₈halocycloalkyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl; each of methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, —C₃-C₈halocycloalkyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl is optionally substituted with at least one substituent —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, phenyl, or 5- to 12-membered heteroaryl.

Aspect 17. The compound of any one of the preceding Aspects, wherein R^{11a}, R^{11b}, R^{12a} and R^{12b} are each independently selected from hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN; each of methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl and pyrazinyl is optionally substituted with at least one substituent R^{11e};
R^{11e}, at each occurrence, is independently hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —OR^{11g} or —CN;
R^{11g} is independently selected from hydrogen, methyl, ethyl, propyl, butyl, pentyl, hexyl, heptyl, octyl;
preferably, R^{11a}, R^{11b}, R^{12a} and R^{12b} are each independently selected from H, —CH₃, —CF₃, —CH₂CH₃, —CH₂CF₃, n-propyl, iso-propyl, n-butyl, iso-butyl, sec-butyl, tert-butyl, pentyl, cyclopropyl, cyclobutyl, cyclopentyl, cyclohexyl, cycloheptyl, pyrrolidinyl, tetrahydrofuranyl, oxazolidinyl, piperidinyl, piperazinyl, morpholinyl, tetrahydropyranyl, phenyl, benzyl, vinyl, propenyl, allyl, but-3-en-1-yl, ethynyl, prop-2-yn-1-yl, prop-1-yn-1-yl, but-3-yn-1-yl, but-2-yn-1-yl, but-1-yn-1-yl,

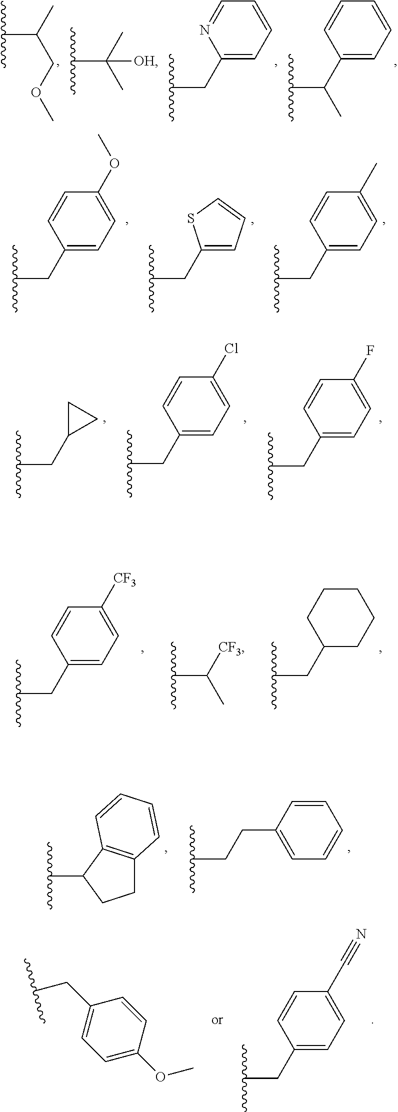

Aspect 18. The compound of any one of the preceding Aspects, wherein (R^{11a} and R^{11b}), (R^{12a}and R^{12b}), (R^{11a} and R^{12a}), (R^{11a} and R^{12b}), (R^{11b} and R^{12a}) or (R^{11b} and R^{12b}) together with the carbon atoms to which they are attached, form a 3-, 4-, 5-, 6- or 8-membered unsaturated or saturated ring, said ring comprising 0, 1, 2 or 3 heteroatoms independently selected from nitrogen, oxygen or sulfur; said ring is optionally substituted with at least one substituent R^{11f};
R^{11f} at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo (═O), —OR^{11g}, thioxo (═S), —SR^{11g}, —CN, —SO₂R^{11g}, —SO₂NR^{11g}R^{11h}, —COR^{11g}, —CO₂R^{11h}, —CONR^{11g}R^{11h}, —NR^{11g}R^{11h}, —NR^{11g}COR^{11h}, —NR^{11g}CO₂R^{11h} or —NR^{11g}SO₂R^{11h}.

R^{11g} and R^{11h} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl;
at each occurrence, R^{11h} is independently —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, —C₆-C₁₂aryl, or 5- to 12-membered heteroaryl;
preferably, (R^{11a} and R^{11b}), (R^{12a} and R^{12b}), (R^{11a} and R^{12a}), (R^{11a} and R^{12b}), (R^{11b} and R^{12a}) or (R^{11b} and R^{12b}) together with the carbon atoms to which they are attached, form a 3-, 4-, 5-, 6- or 8-membered unsaturated or saturated ring;
more preferably, (R^{11a} and R^{11b}), (R^{12a} and R^{12b}), (R^{11a} and R^{12a}), (R^{11a} and R^{12b}), (R^{11b} and R^{12a}) or (R^{11b} and R^{12b}) together with the carbon atoms to which they are attached, form a 3-, 4-, 5-, 6- or 8-membered saturated ring;

Aspect 19. The compound of any one of the preceding Aspects, wherein R¹³, R^{14a} and R^{14b}are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one substituent R^{13a};
R^{13a}, at each occurrence, is independently halogen, —OH, —CN, oxo, methyl, ethyl, propyl, butyl, pentyl, hexyl, heptyl, octyl, cyclopropyl, cyclobutyl, cyclopentyl, cyclohexyl, cycloheptyl, cyclooctyl, —C₁₋₈haloalkyl, —C₁₋₈alkoxy, —C₂₋₈alkenyl, —C₂₋₈alkynyl, —C₃-C₈halocycloalkyl, 3- to 8-membered heterocyclyl, phenyl, or 5- to 12-membered heteroaryl;
preferably, R¹³, R^{14a} and R^{14b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl;
more preferably, R¹³ is hydrogen, methyl, ethyl, propyl or butyl; R^{14a} and R^{14b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl; even more preferably, R¹³ is hydrogen; R^{14a} and R^{14b} are each independently selected from hydrogen, methyl, ethyl, propyl, butyl, cyclopropyl, cyclobutyl, cyclopentyl.

Aspect 20. The compound of any one of the preceding Aspects, wherein R¹ is selected from

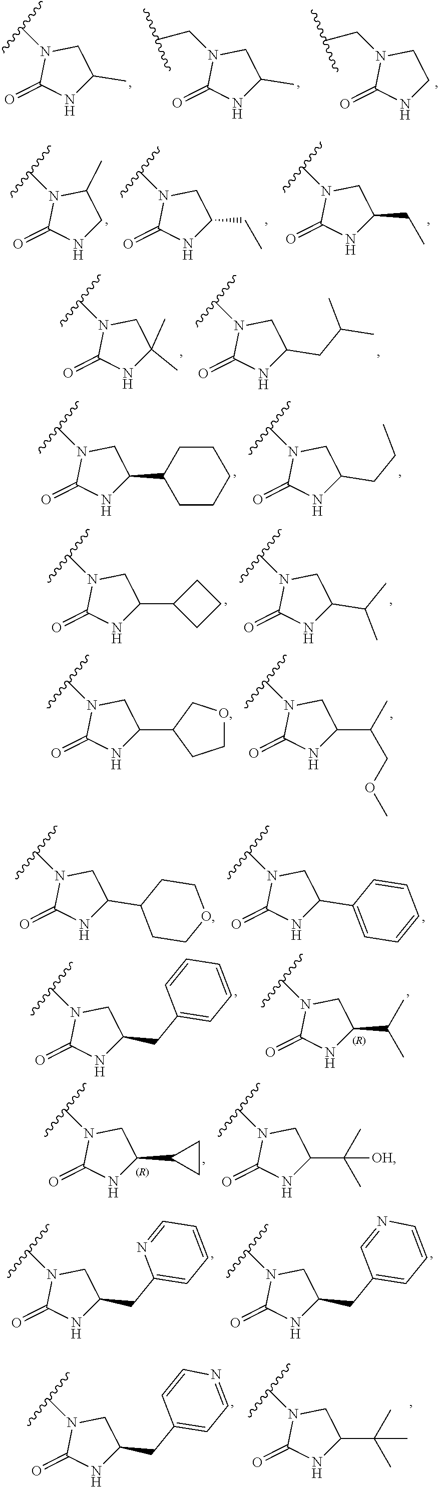

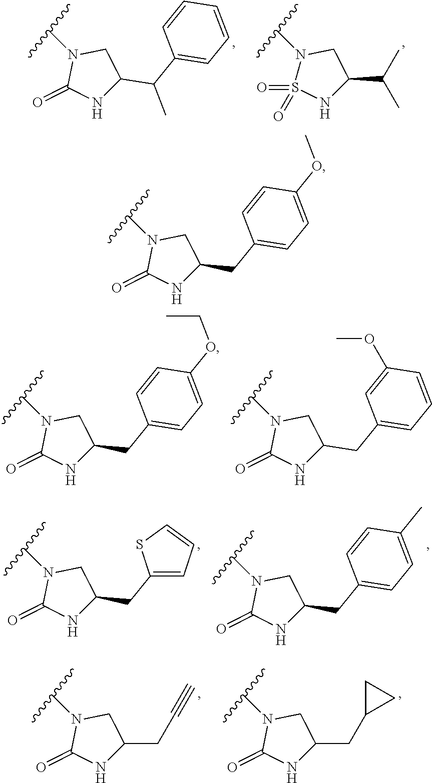

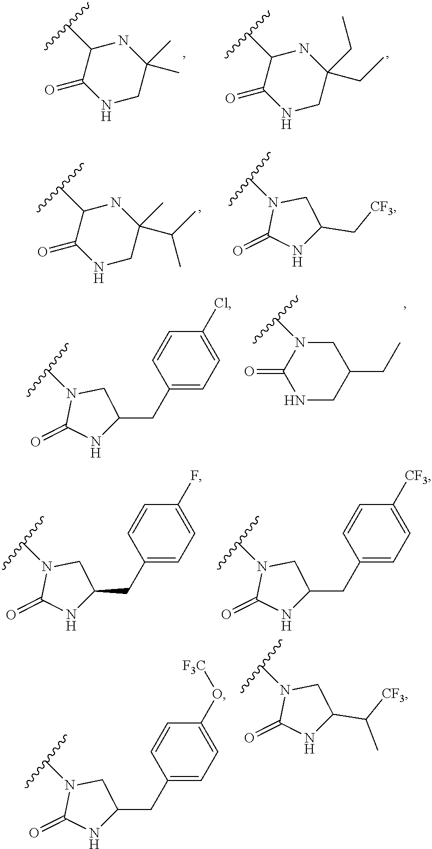

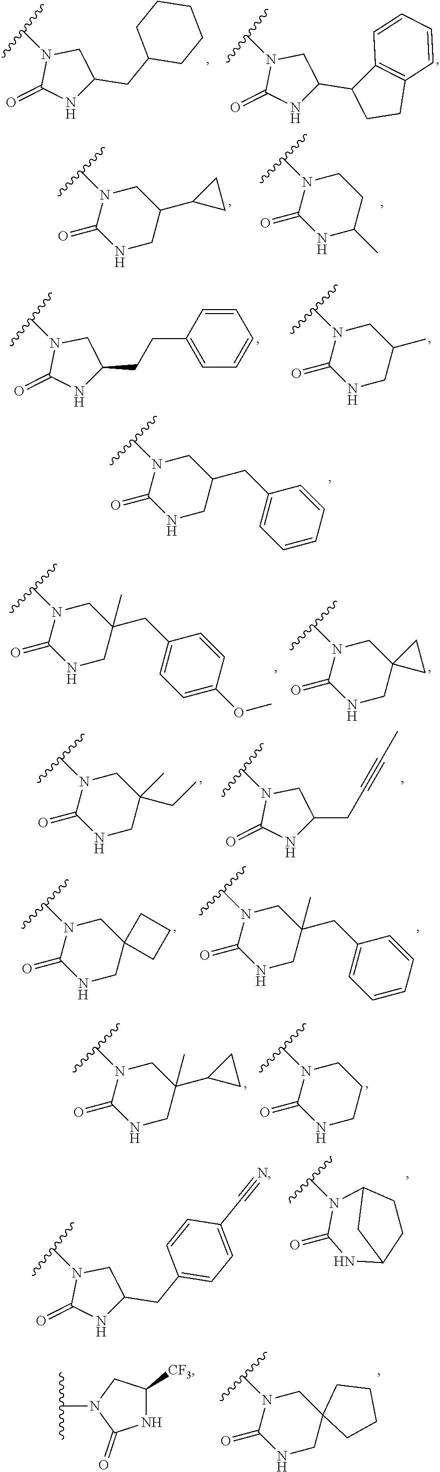

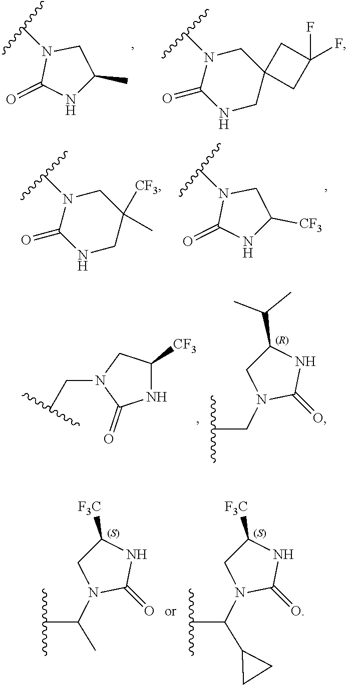

Aspect 21. The compound of any one of the preceding Aspects, wherein R² is independently selected from H, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —CN, —COR^{2a}, —CO₂R^{2a}, —CONR^{2a}R^{2b}, —NR^{2a}R^{2b}, —NR^{2a}COR^{2b}, each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl and imidazopyridinyl is optionally substituted with at least one substituent R^{2c};
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2d}; or
R^{2a} and R^{2b} together with the carbon atoms to which they are attached, form a 3-, 4-, 5-, 6-, 7- or 8-membered unsaturated or saturated ring, said ring comprising 0, 1, 2, 3 heteroatoms independently selected from nitrogen, oxygen or sulfur; said ring is optionally substituted with at least one substituent R^{2d};
R^{2c} and R^{2d}, at each occurrence, are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, oxo (═O), —OR^{2e}, —SR^{2e}, —CN, —COR^{2e}, —CO₂R^{2e}, —CONR^{2e}R^{2f}, —NR^{2e}R^{2f}, —NR^{2e}COR^{2f} or —NR^{2e}CO₂R^{2f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2g};
R^{2e} and R^{2f}are each independently selected from hydrogen, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{2h}
R^{2g} and R^{2h}, at each occurrence, are each independently —F, —Cl, —Br, —I, —OH, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, phenyl, or 5- to 12-membered heteroaryl.

Aspect 22. The compound of any one of the preceding Aspects, wherein R² is independently selected from H, methyl, ethyl, propyl, butyl, pentyl, hexyl, cyclopropyl, cyclobutyl, cyclopentyl, cyclohexyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —CN, —COR^{2a}, —CO₂R^{2a} or —CONR^{2a}R^{2b}, each of methyl, ethyl, propyl, butyl, pentyl, hexyl, cyclopropyl, cyclobutyl, cyclopentyl, cyclohexyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2c};
R^{2a} and R^{2b} are each independently selected from hydrogen, methyl, ethyl, propyl, butyl, pentyl, hexyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl; each of methyl, ethyl, propyl, butyl, pentyl, hexyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2d}; or
R^{2a} and R^{2b} together with the carbon atoms to which they are attached, form a 3-, 4-, 5- or 6-membered unsaturated or saturated ring, said ring comprising 0 or 1 heteroatoms independently selected from nitrogen, oxygen or sulfur; said ring is optionally substituted with at least one substituent R^{2d};
R^{2c} and R^{2d}, at each occurrence, are each independently hydrogen (H or D), —F, —Cl, —Br, —I, methyl, ethyl, propyl, butyl, pentyl, hexyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —OR^{2e}, —CN or —NR^{2e}R^{2f}; each of methyl, ethyl, propyl, butyl, pentyl, hexyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2g};
R^{2e} and R^{2f}are each independently selected from hydrogen, methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl or phenyl; each of methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl or phenyl is optionally substituted with at least one substituent R^{2h}
R^{2g} and R^{2h}, at each occurrence, are each independently —F, —Cl, —Br, —I, —OH, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl;
preferably, R² is

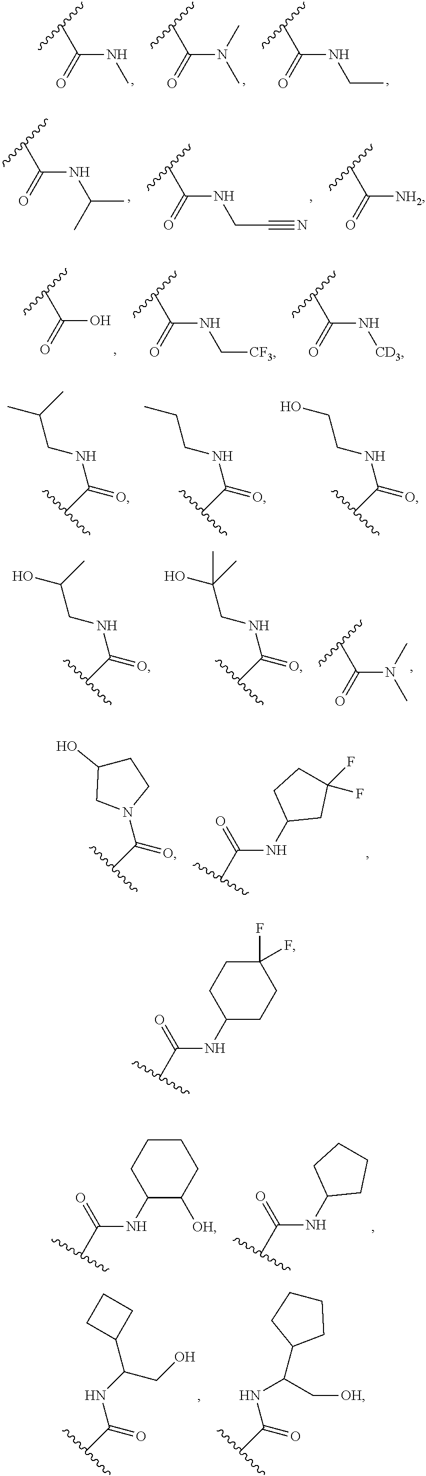

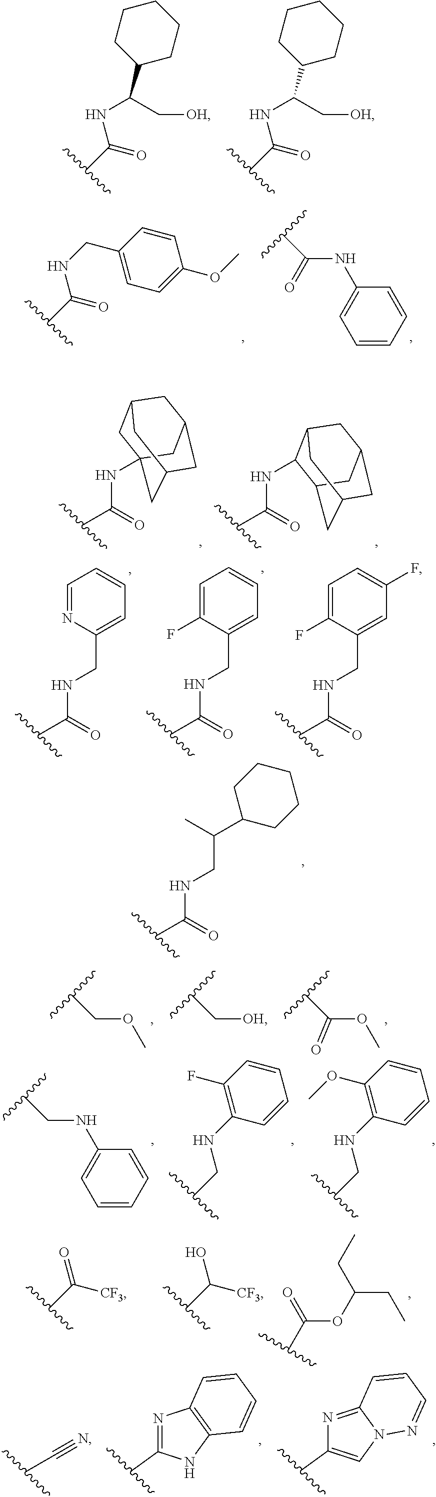

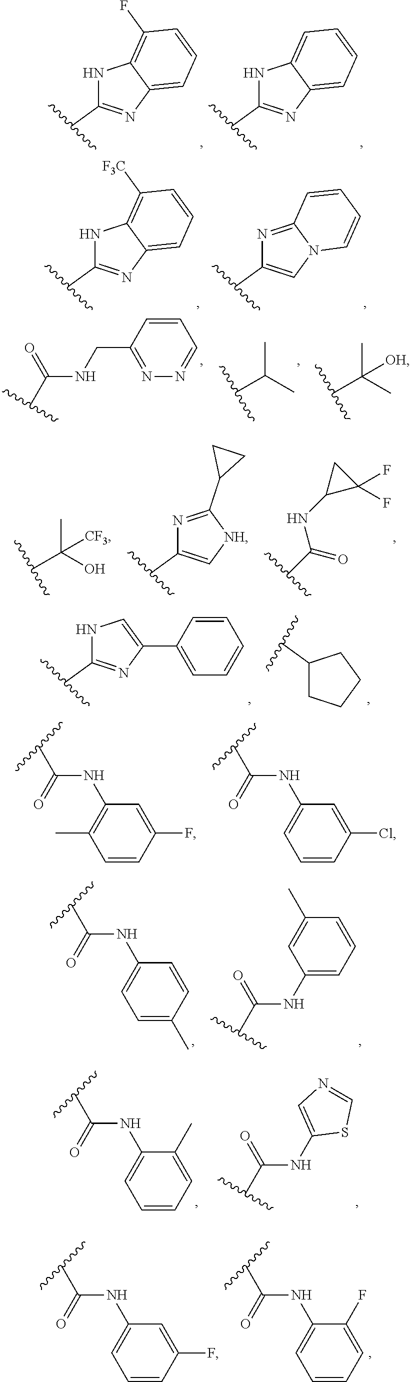

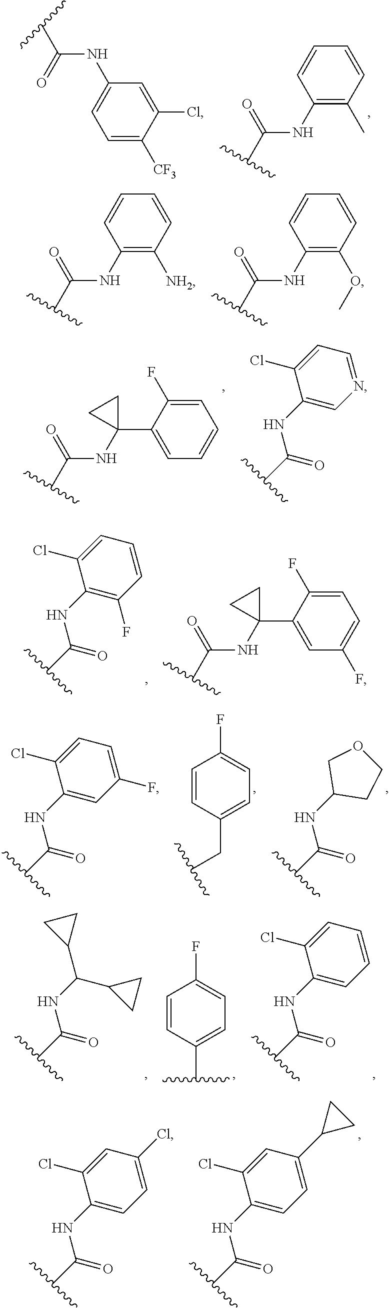

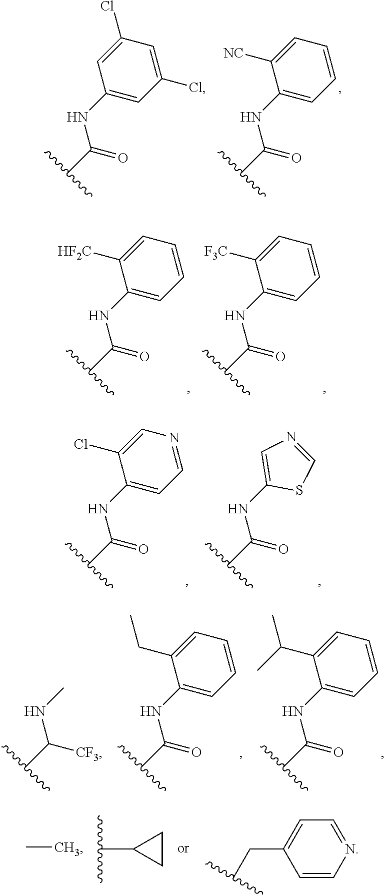

Aspect 23. The compound of any one of the preceding Aspects, wherein R³ is independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl or —CN, wherein each of methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl is optionally substituted with at least one substituent selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl;
preferably, R³ is independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl or —CN; more preferably, R³ is independently selected from hydrogen, —F, —Cl, —Br, —I.

Aspect 24. The compound of any one of the preceding Aspects, wherein R⁴ is oxo (═O).

Aspect 25. The compound of any one of the preceding Aspects, wherein R³ and R⁴ together with the carbon atoms to which they are attached, form a 5- to 6-membered aromatic ring, said ring comprising 1, 2 or 3 heteroatoms independently selected from nitrogen, oxygen or sulfur; said ring is optionally substituted with at least one substituent R^{3a};
R^{3a} is independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, —CN, wherein each of methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl is optionally substituted with at least one substituent selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl.

Aspect 26. The compound of any one of the preceding Aspects, wherein R⁵ is phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl, imidazopyridinyl, —COR^{5a}, —CO₂R^{5a}, or —CONR^{5a}R^{5b}, wherein each of phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5c};
R^{5a} and R^{5b} are each independently selected from methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, oxadiazolyl, triazolyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, oxadiazolyl, triazolyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5d}; or
R^{5a} and R^{5b} together with the nitrogen atom to which they are attached, form a 3-, 4-, 5-, 6-, 7- or 8-membered unsaturated or saturated ring, said ring comprising 0, 1, 2 or 3 heteroatoms independently selected from nitrogen, oxygen or sulfur; said ring is optionally substituted with at least one substituent R^{5d};
R^{5c} and R^{5d}, at each occurrence, are independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, oxo (═O), —OR^{5e}, —SR^{5e}, —CN, —COR^{5e}, —CO₂R^{5e}, —CONR^{5e}R^{5f}, —NR^{5e}R^{5f}, —NR^{5e}COR^{5f} or —NR^{5e}CO₂R^{5f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl or imidazopyridinyl is optionally substituted with at least one substituent R^{5g};
R^{5e} and R^{5f}are each independently selected from hydrogen, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₁₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5h};
R^{5g} and R^{5h} are each independently selected from halogen, —OH, —CN, methyl, ethyl, propyl, butyl, pentyl, hexyl, heptyl, octyl, methoxy, ethoxy, propoxy, butoxy, pentoxy, hexoxy, heptoxy, octoxy, C₁₋₈ alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 27. The compound of any one of the preceding Aspects, wherein R⁵ is pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl, wherein each of pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl, imidazopyridinyl, is optionally substituted with at least one substituent R^{5c};
R^{5c}, at each occurrence, is independently selected from hydrogen, —F, —Cl, —Br, —I, methyl, ethyl, propyl, butyl, pentyl, hexyl, cyclopropyl, cyclobutyl, cyclopentyl, cyclohexyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl;
preferably, R is benzooxazolyl;
more preferably, R⁵ is

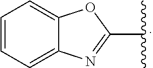 [SMILES: *c1nc2ccccc2o1]

Aspect 28. The compound of any one of the preceding Aspects, wherein R⁵ is —COR^{5a}, CO₂R^{5a}, or —CONR^{5a}R^{5b};
wherein R^{5a} and R^{5b} are each independently selected from methyl, ethyl, propyl, butyl, pentyl, hexyl, heptyl, octyl, cyclopropyl, cyclobutyl, cyclopentyl, cyclohexyl, cycloheptyl, cyclooctyl, phenyl, oxadiazolyl, triazolyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5d};
R^{5d}, at each occurrence, is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —OR^{5e} or —CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl or imidazopyridinyl is optionally substituted with at least one substituent R^{5g};
R^{5e} is selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5h};
R^{5g} and R^{5h} are each independently selected from halogen, —OH, —CN, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, phenyl or 5- to 12-membered heteroaryl;
preferably, R⁵ is

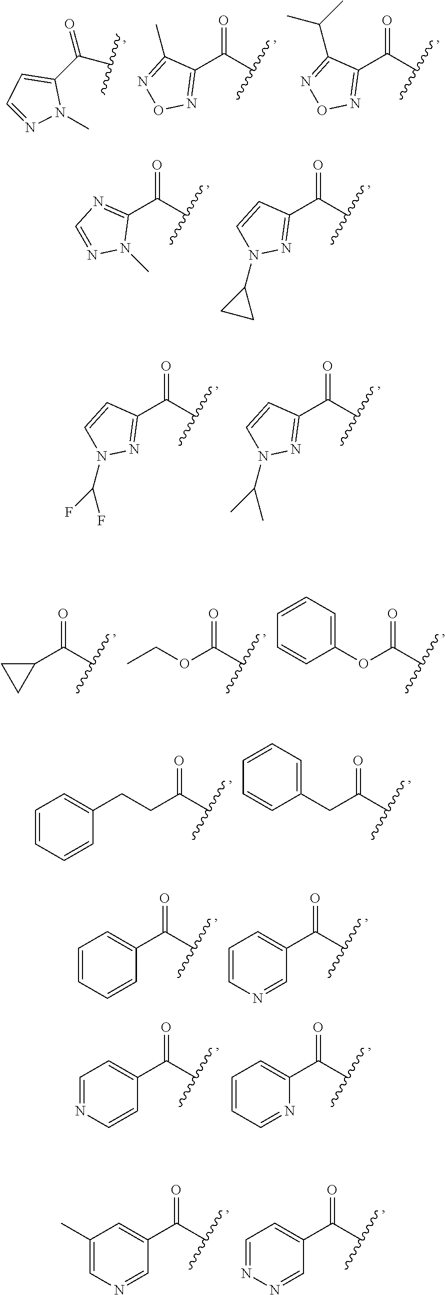

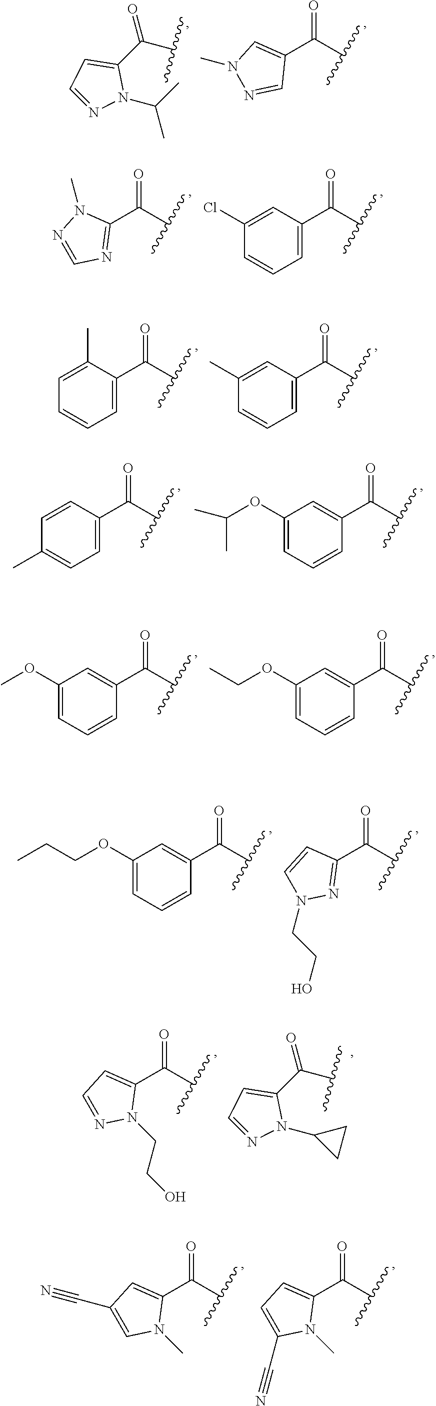

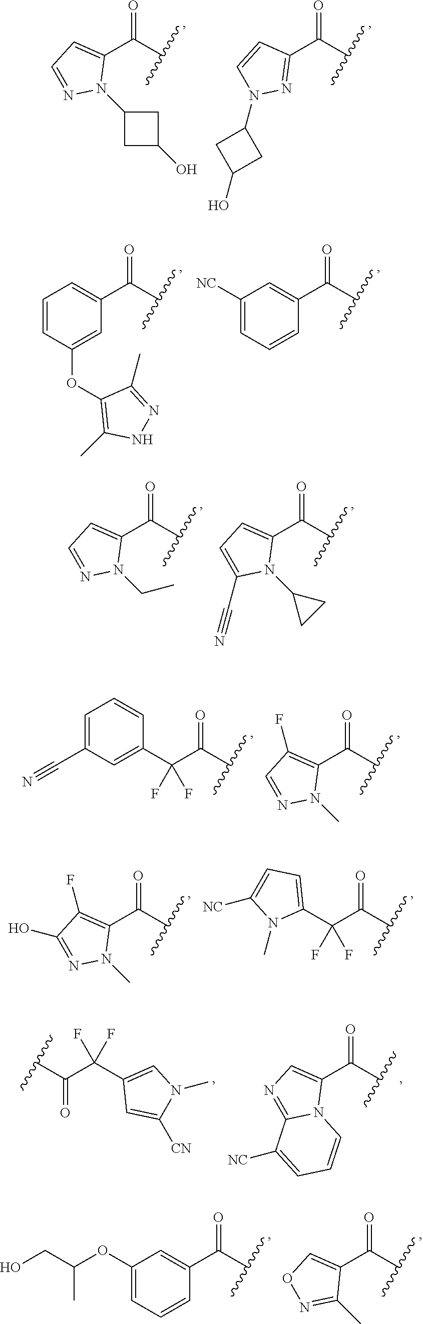

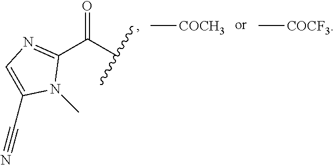

Aspect 29. The compound of any one of the preceding Aspects, wherein R⁶ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN, —OR^{6a}, —NR^{6a}R^{6b}, —COR^{6a}, —C₂R^{6a}, —CONR^{6a}R^{6b}, —COR^{6a} or —NR^{6a}COR^{6b}, each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl is optionally substituted with at least one substituent R^{6c};
R^{6a} and R^{6b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{6d};
R^{6c}, at each occurrence, is independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{6e}, —SR^{6e}, —CN, —COR^{6e}, —CO₂R^{6e}, —CONR^{6e}R^{6f}, —NR^{6e}R^{6f}, —NR^{6e}COR^{6f} or —NR^{6e}CO₂R^{6f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with at least one substituent R^{6g};
R^{6e} and R^{6f} are each independently selected from hydrogen, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{6d} and R^{6g} are each independently selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;

Aspect 30. The compound of any one of the preceding Aspects, wherein R⁶ is H, —F, —Cl, —Br, —I, methyl, —CF₃, ethyl, —CH₂CF₃, —CF₂CH₃, propyl, butyl, pentyl, hexyl, heptyl, octyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN, methoxy, ethoxy, propoxy, butoxy, pentoxy, hexoxy, heptoxy, octoxy,

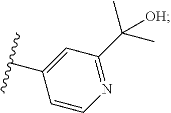 [SMILES: *c1ccnc(C(C)(C)O)c1]

preferably,

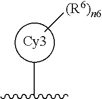

moiety is

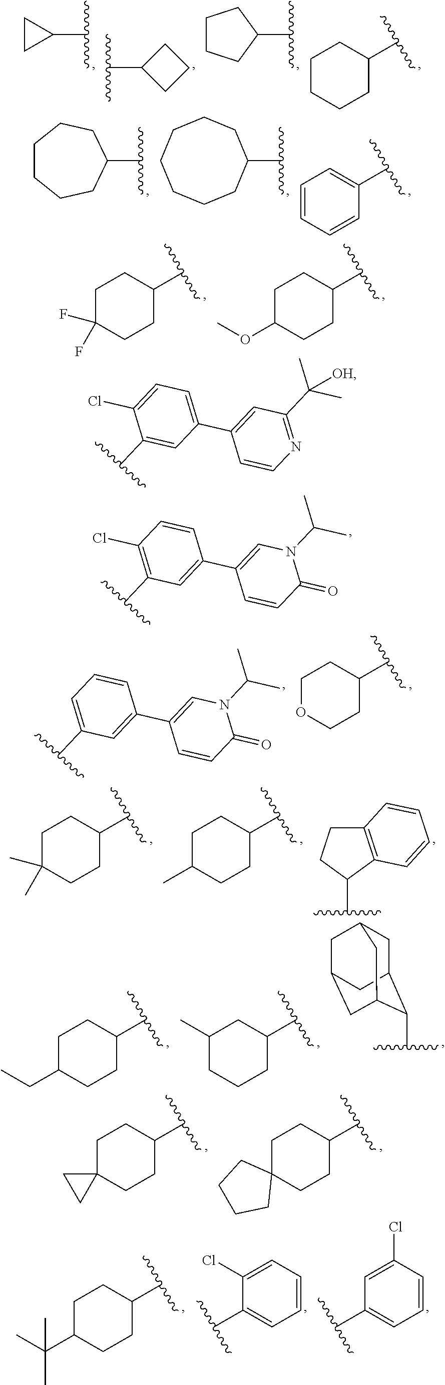

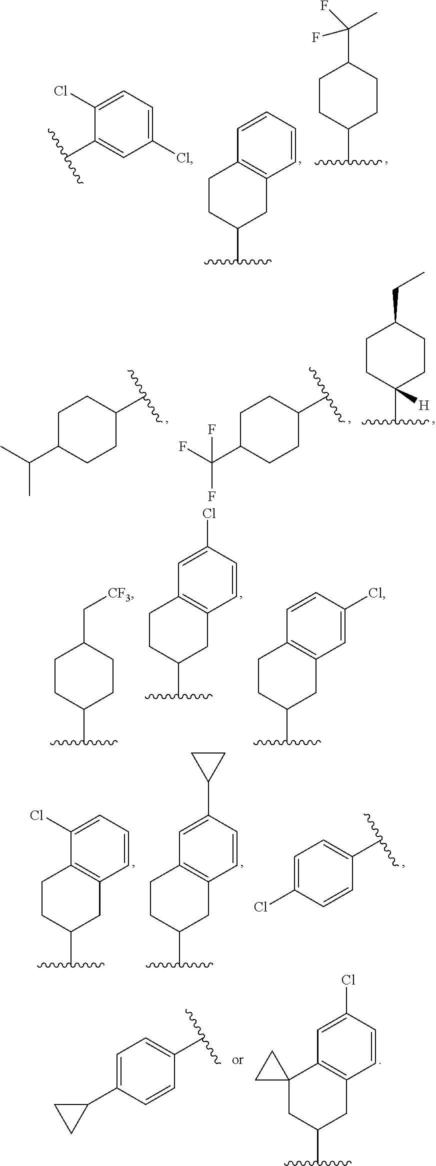

Aspect 31. The compound of any one of the preceding Aspects, wherein R⁹ and R¹⁰ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl or 5- to 12-membered heteroaryl, each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl or 5- to 12-membered heteroaryl is optionally substituted with at least one substituent R^{9a};
R^{9a}, at each occurrence, is independently halogen, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R⁹ and R¹⁰ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl or 5- to 12-membered heteroaryl;
more preferably, R⁹ and R¹⁰ are each independently selected from H, methyl, ethyl, propyl, butyl, pentyl, cyclopropyl, cyclobutyl, cyclopentyl;
more preferably, R⁹ and R¹⁰ are each independently selected from H, cyclopropyl.

Aspect 32. The compound of any one of the preceding Aspects, wherein the compound is selected from

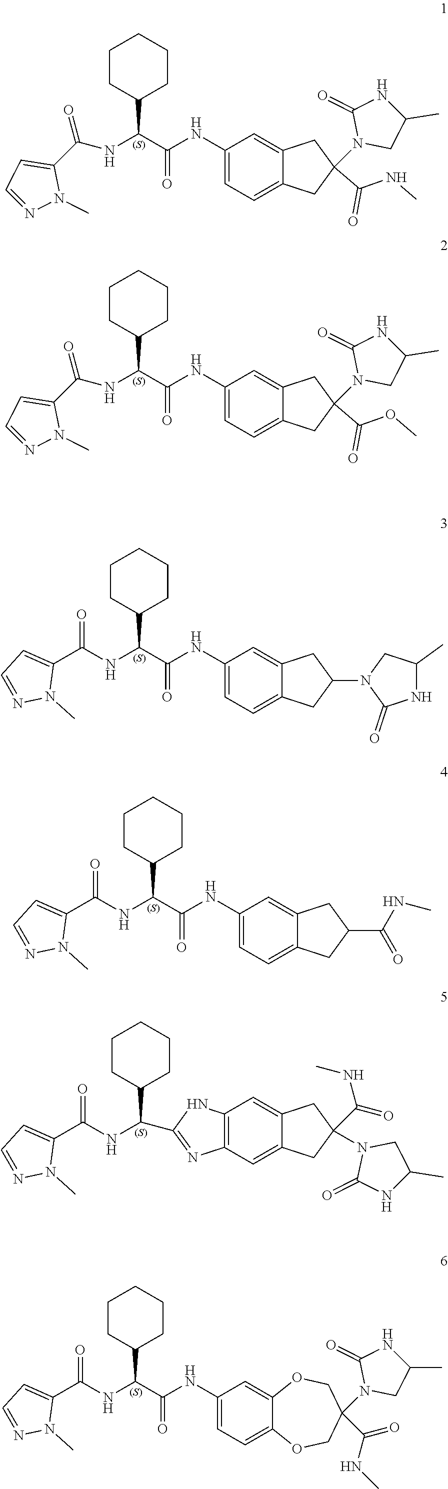

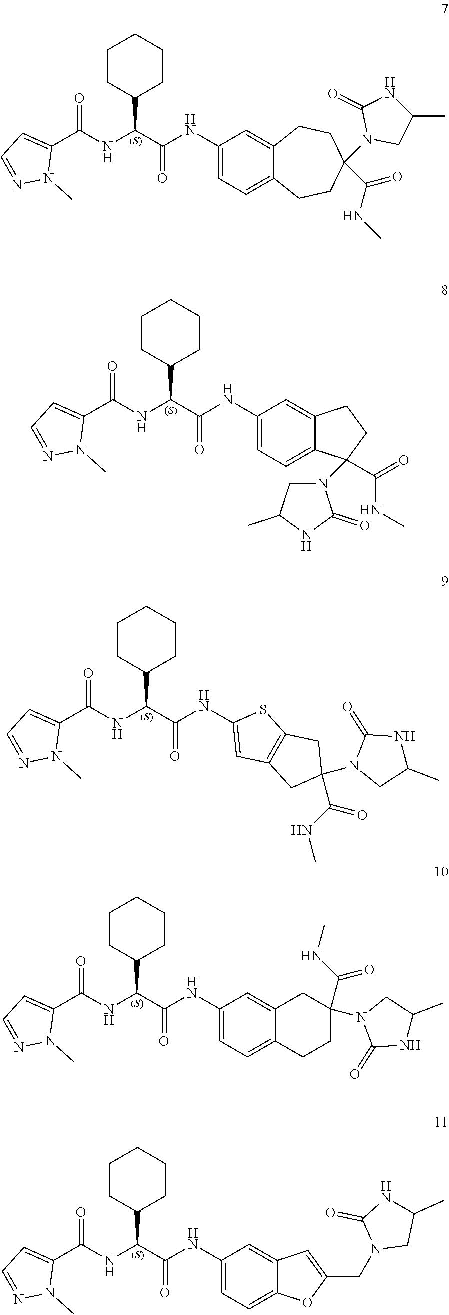

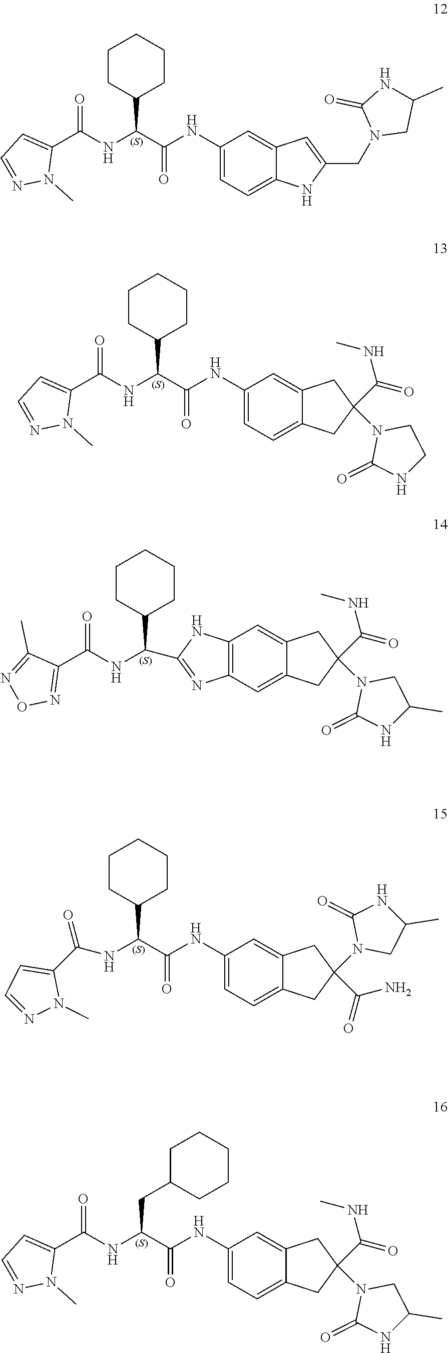

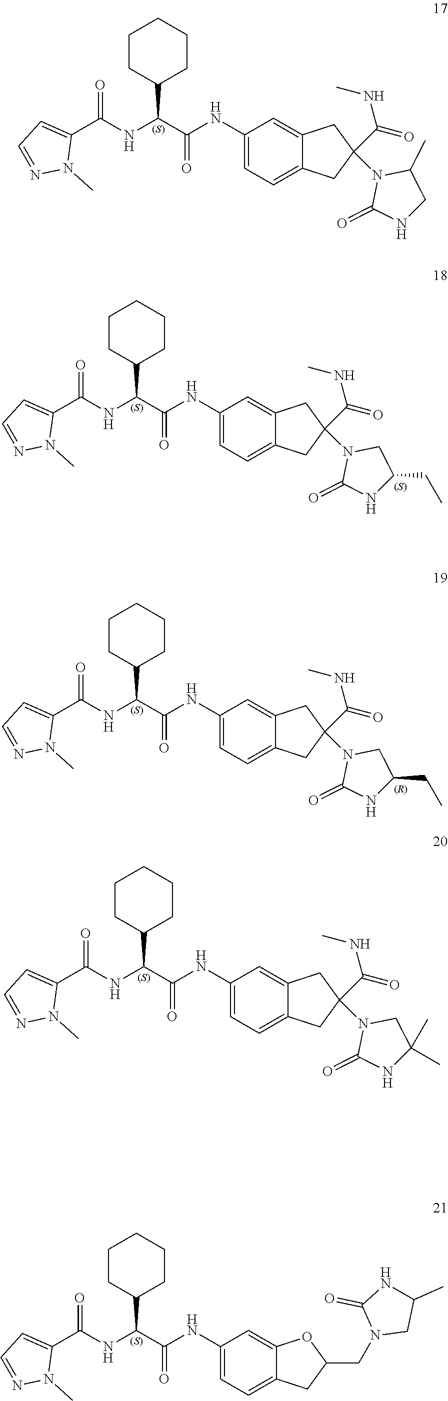

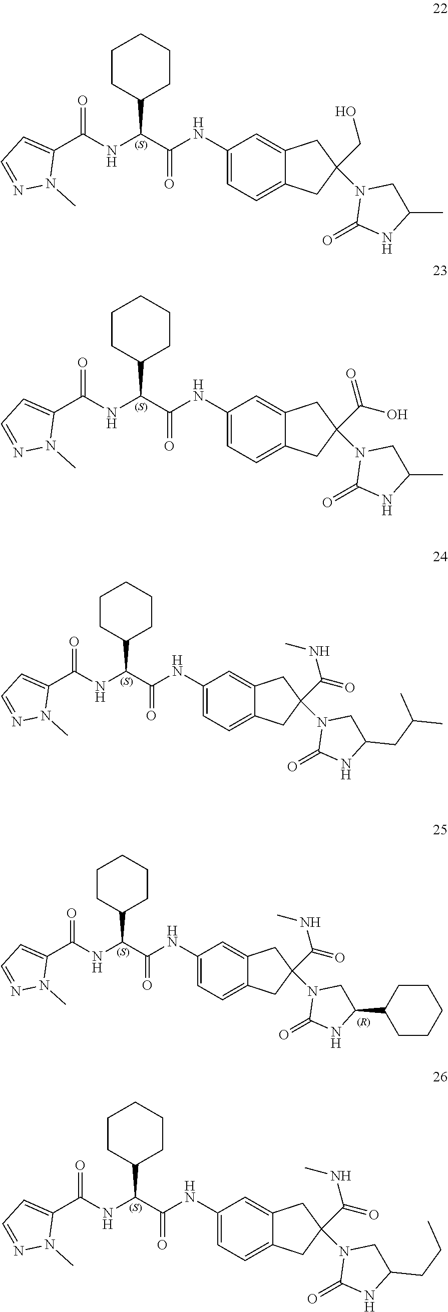

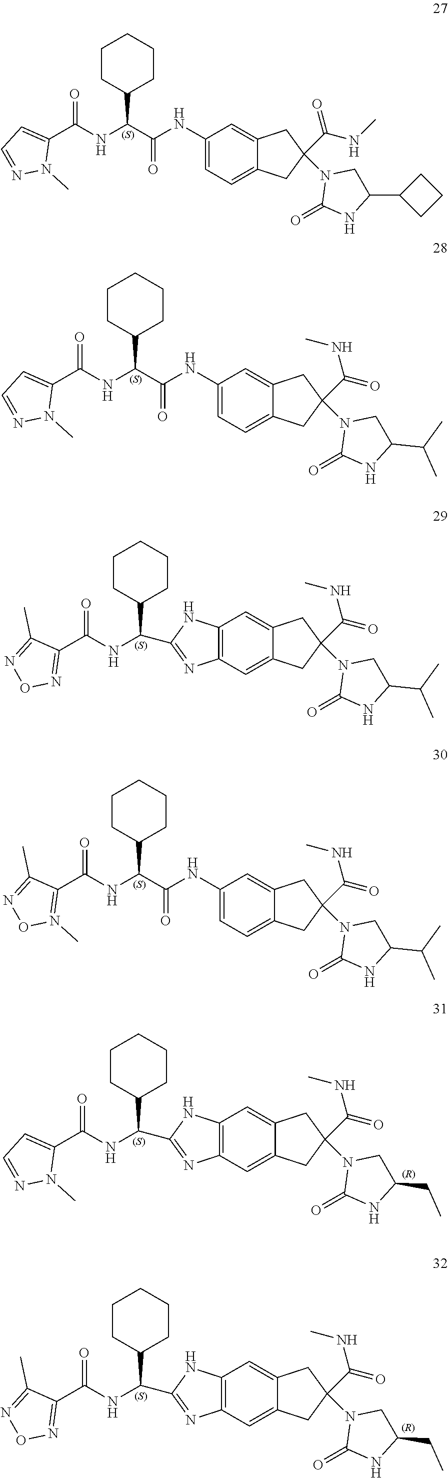

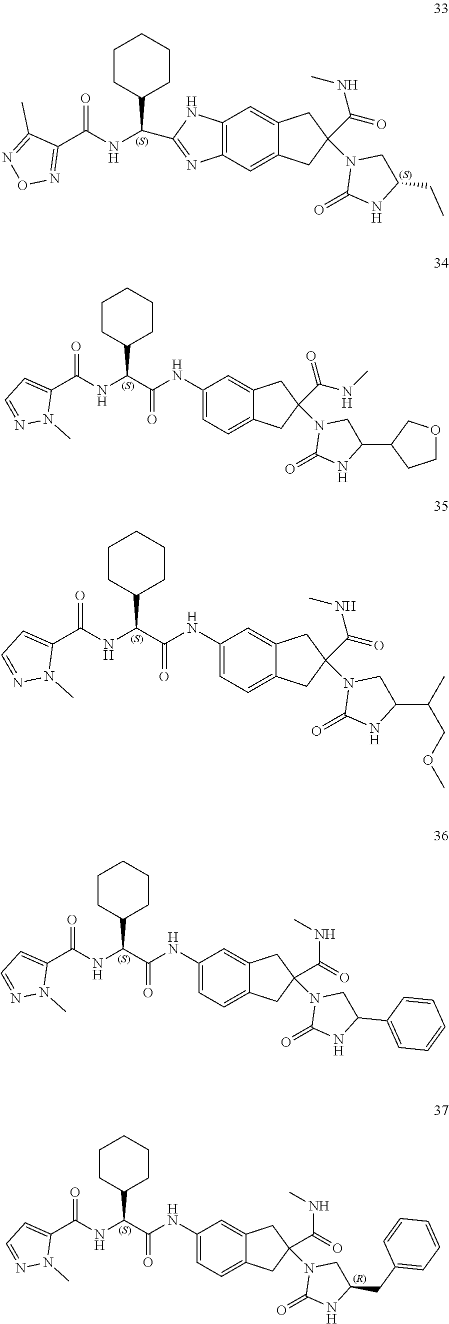

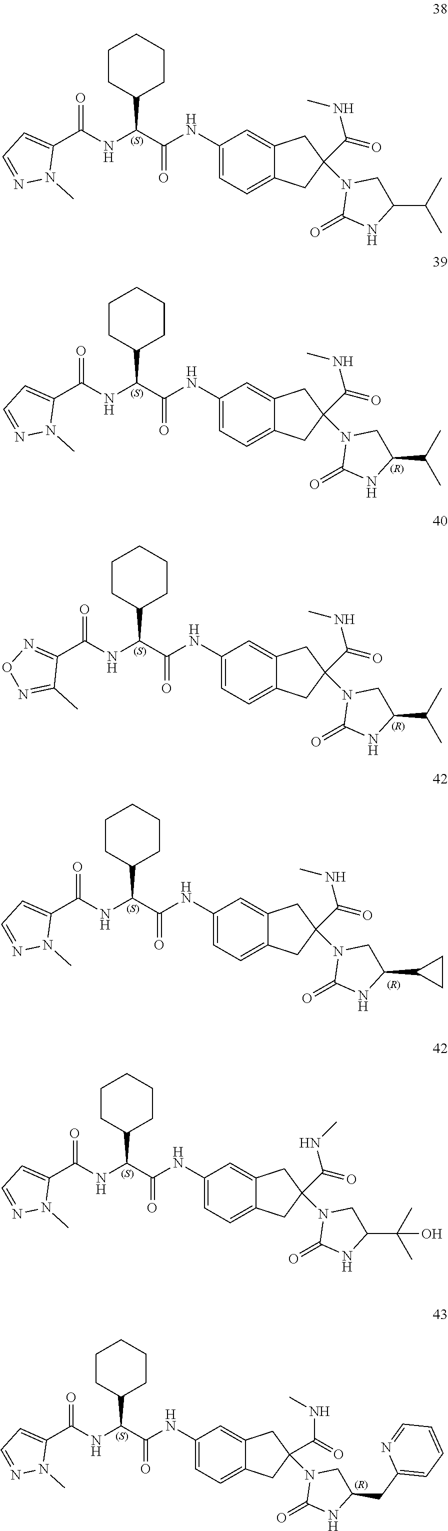

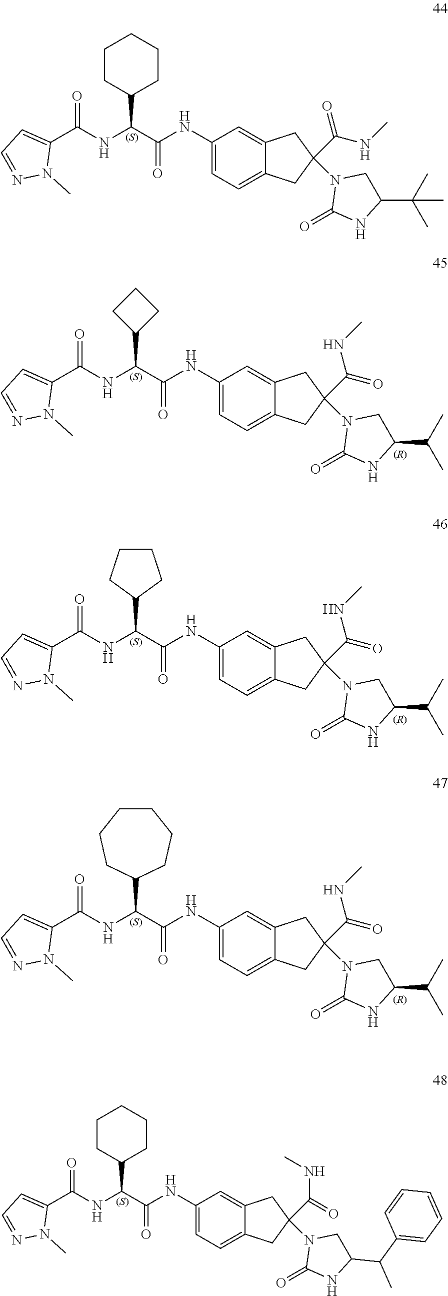

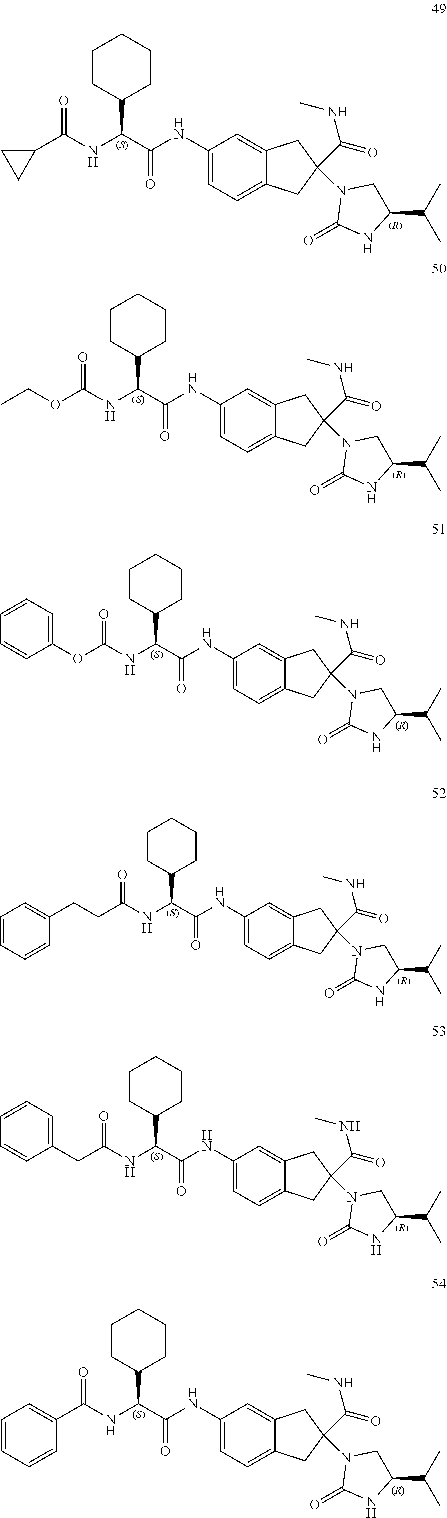

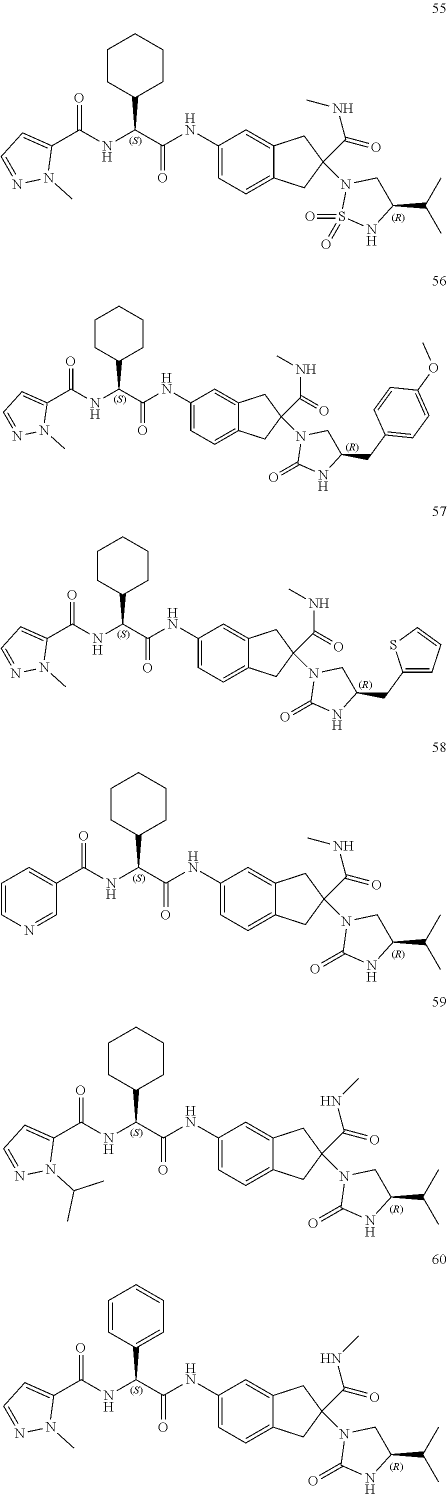

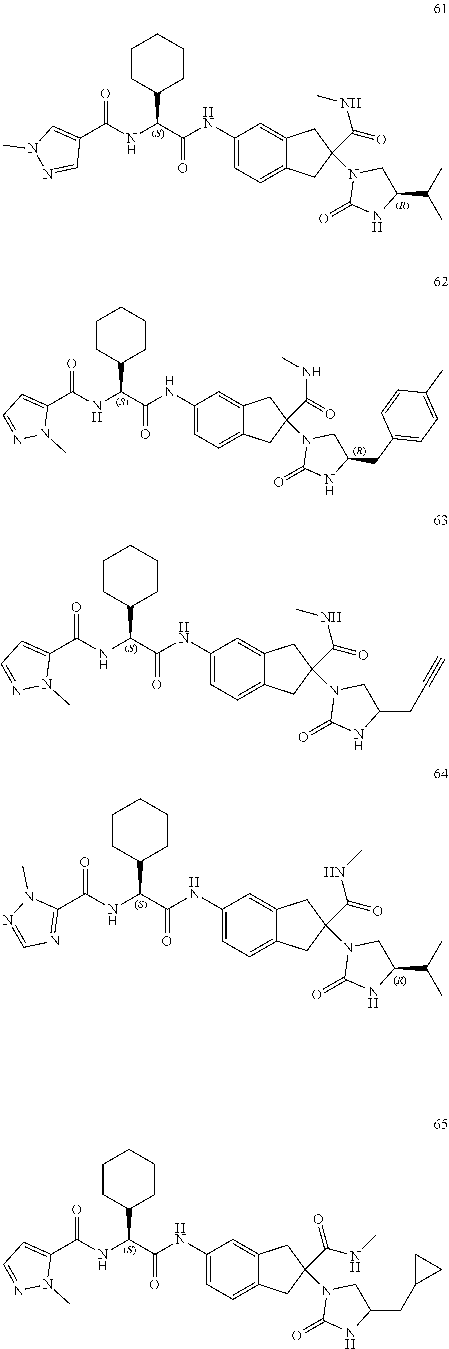

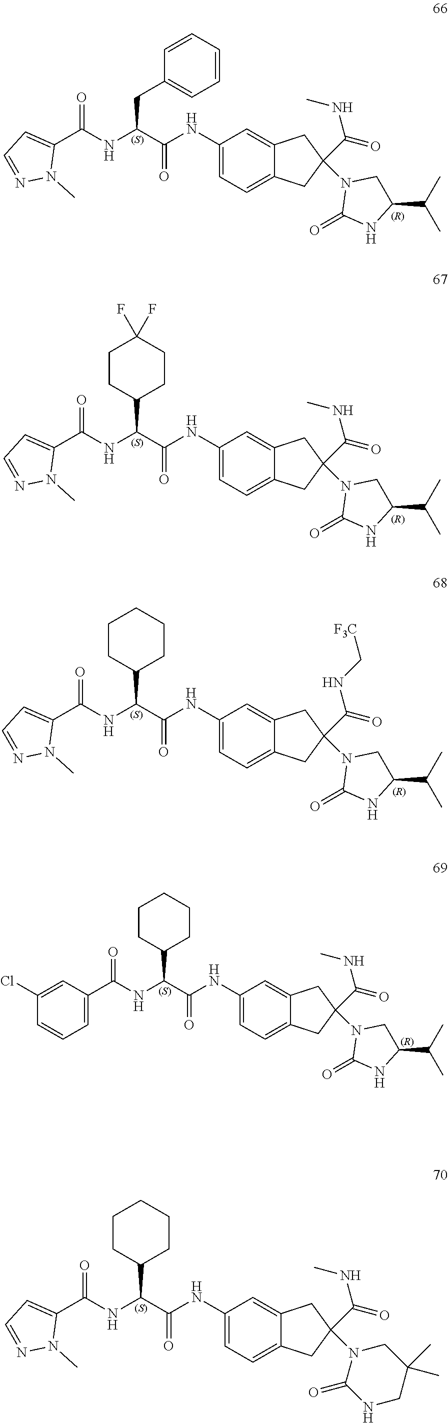

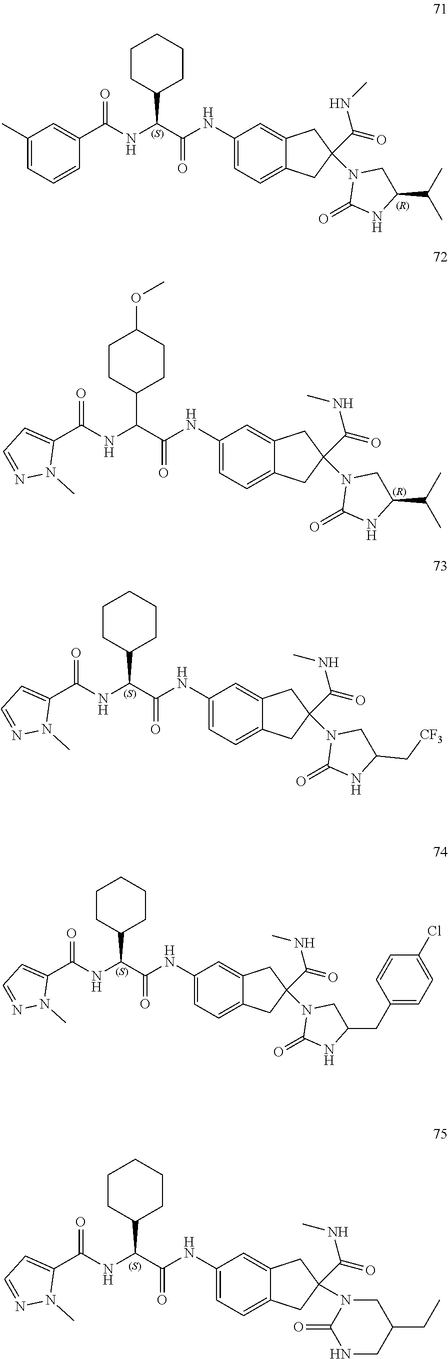

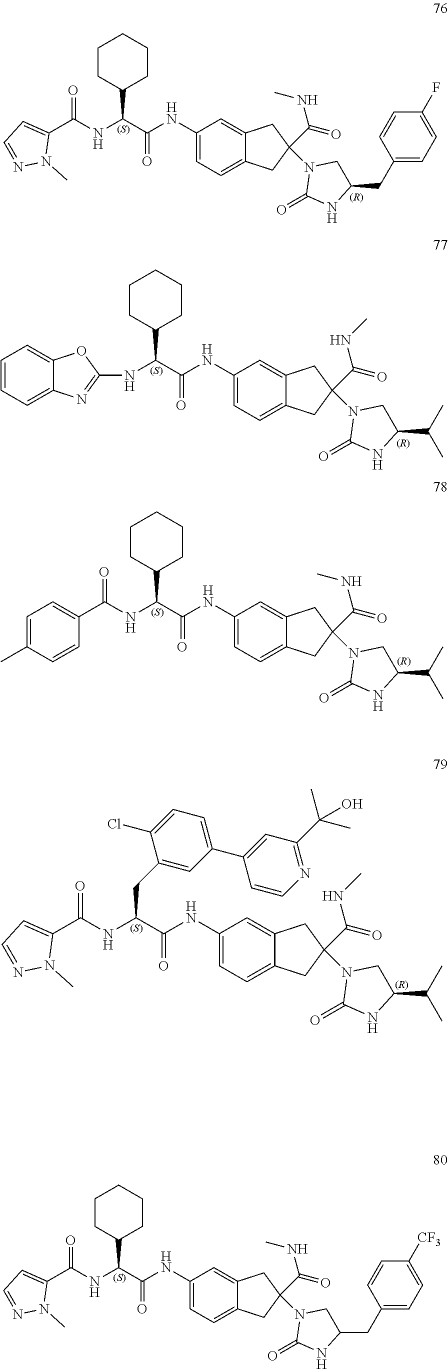

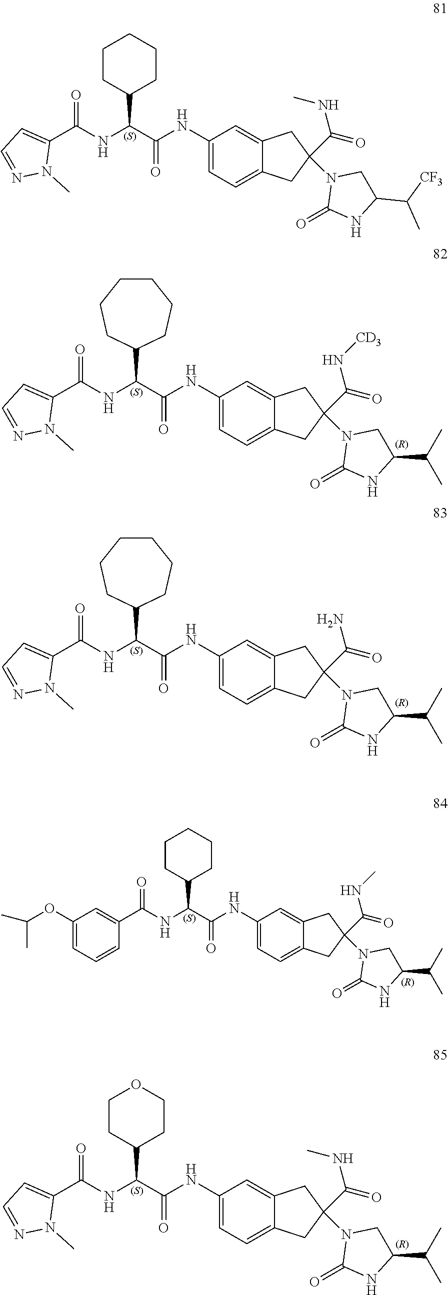

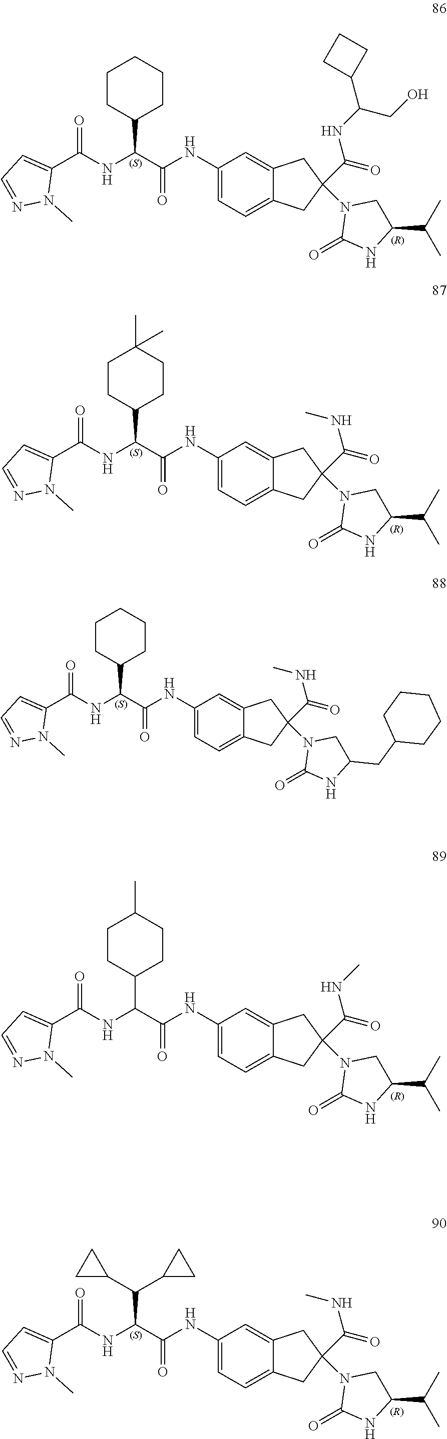

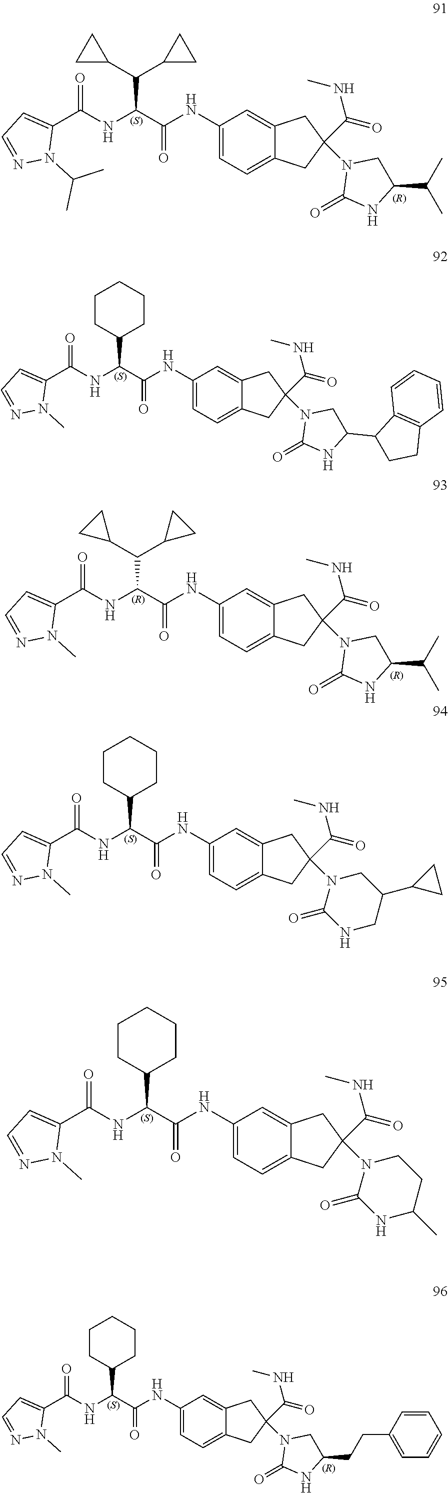

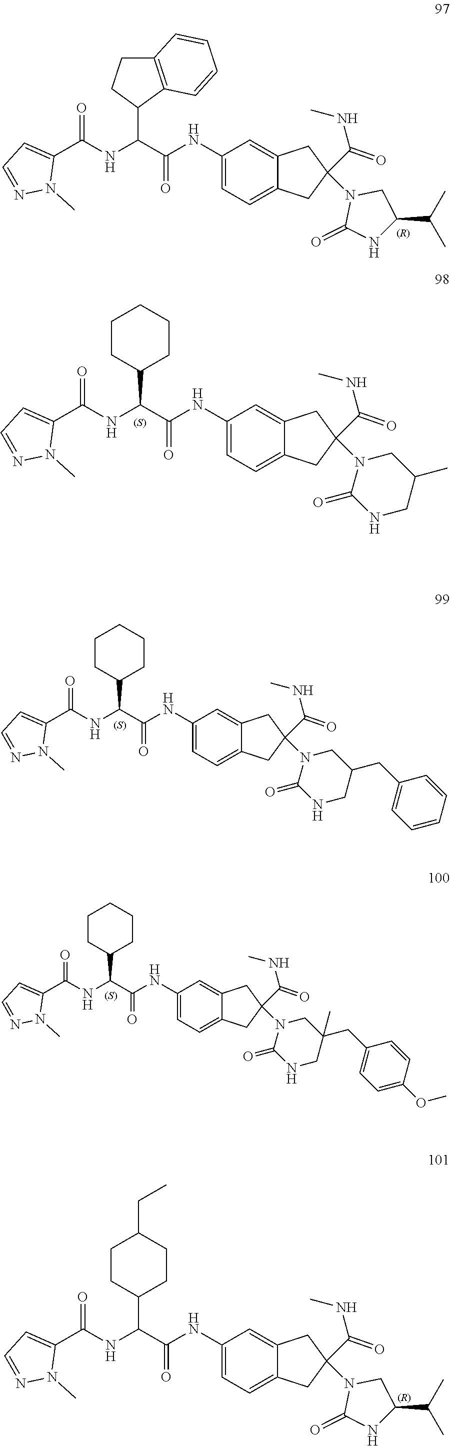

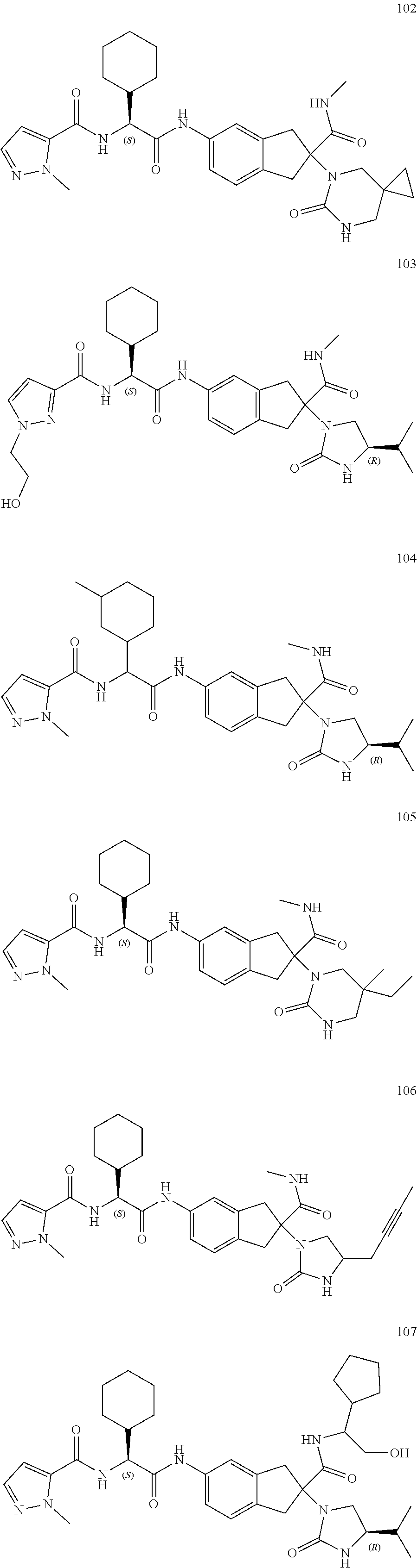

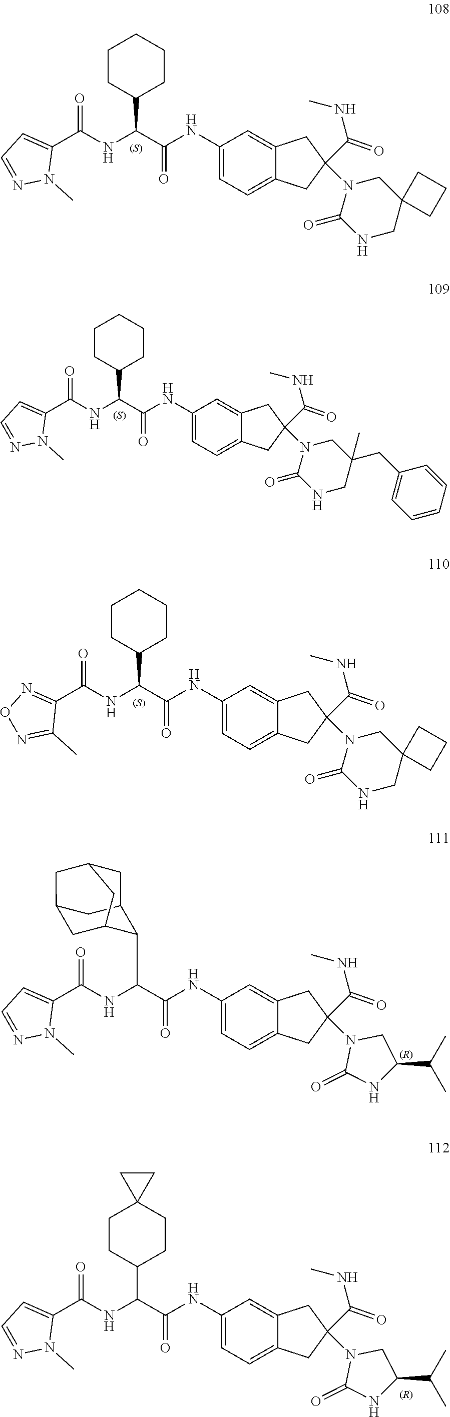

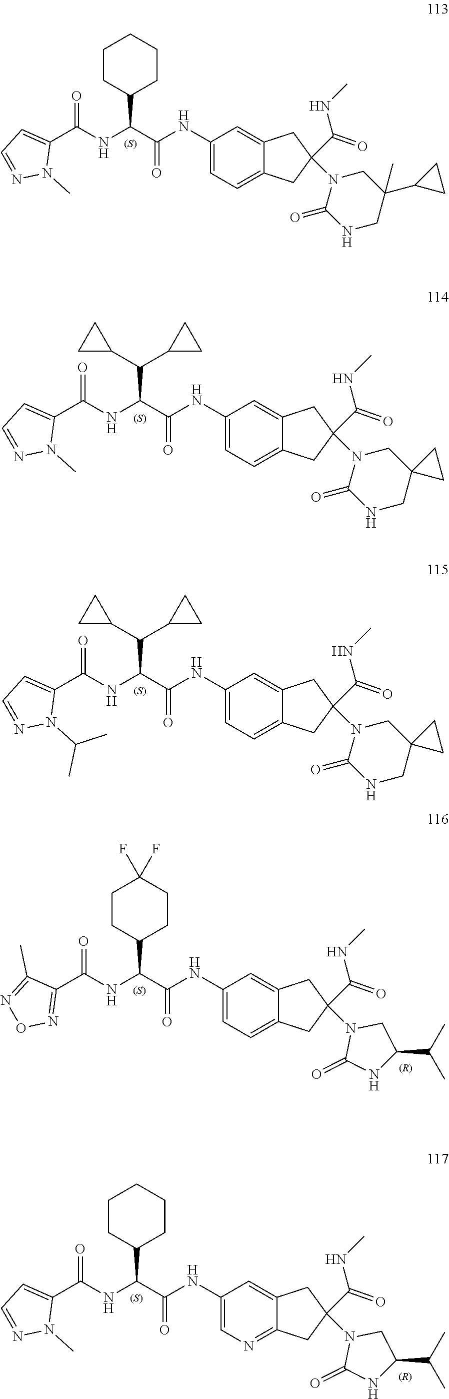

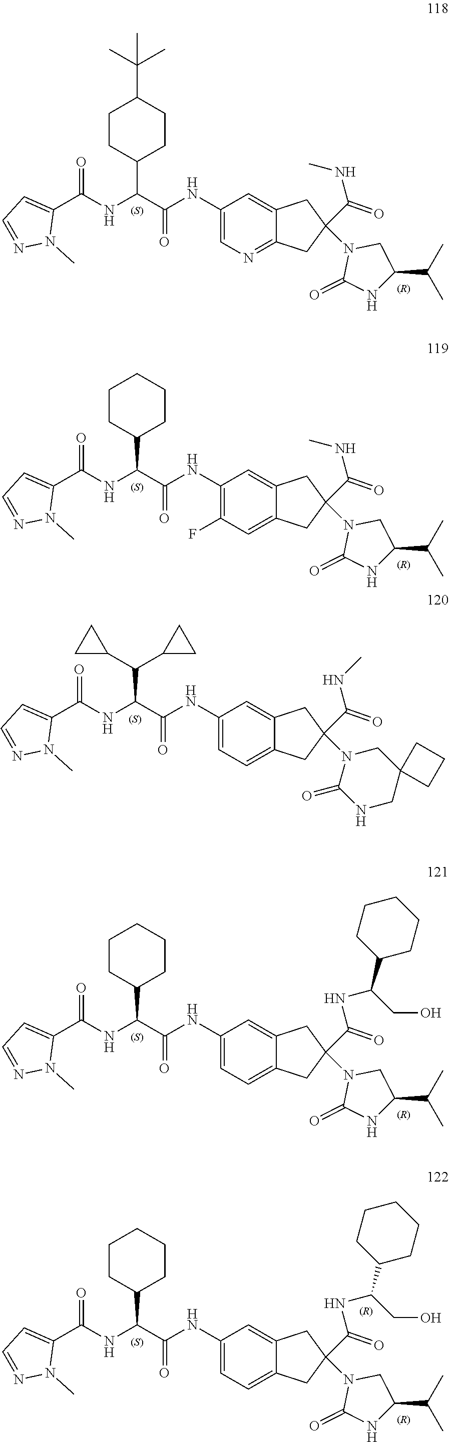

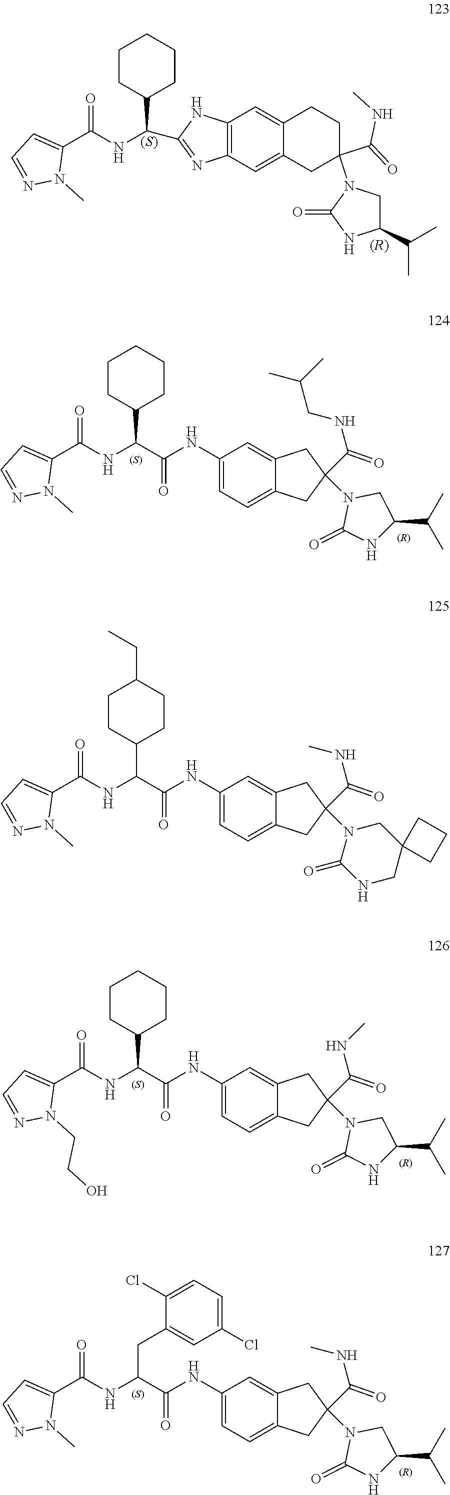

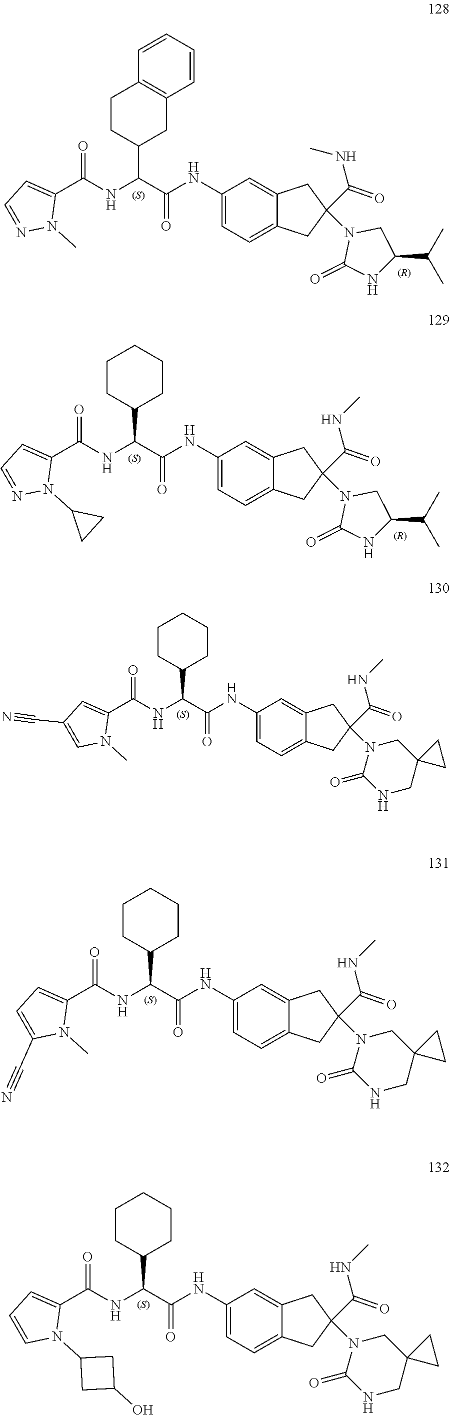

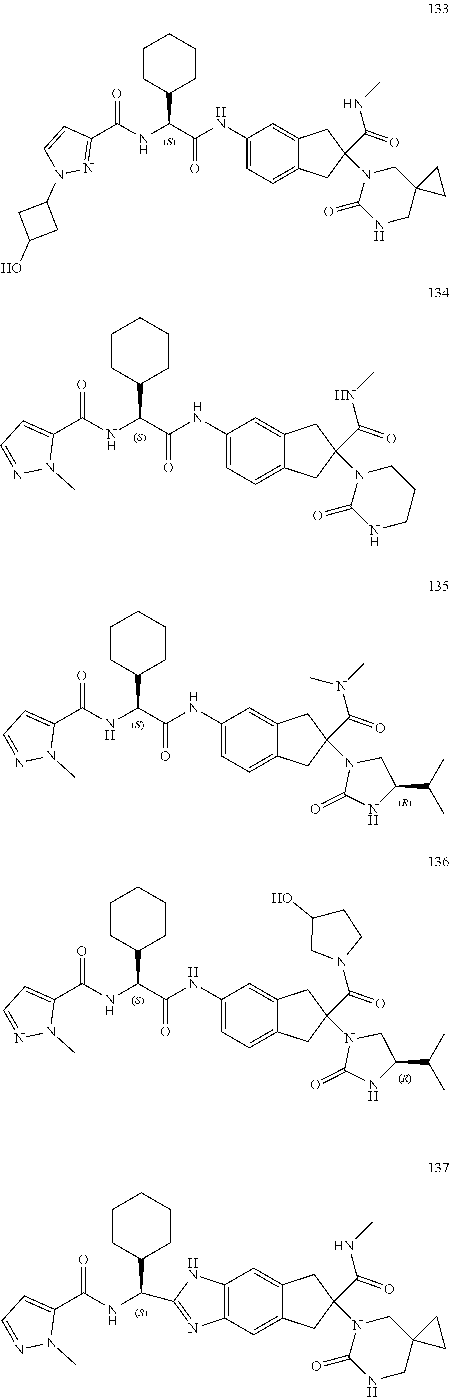

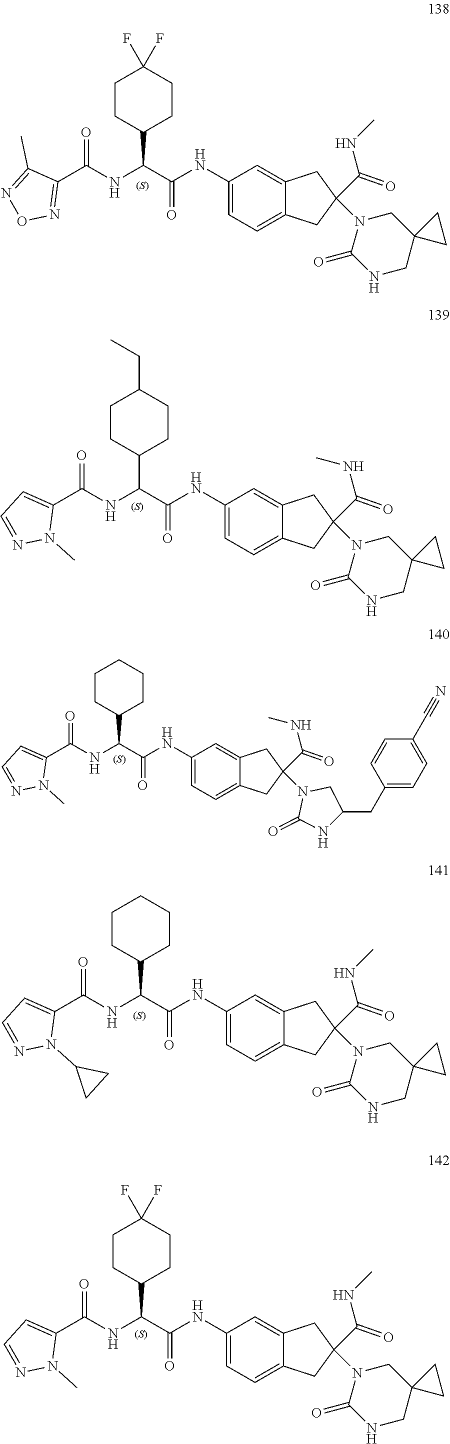

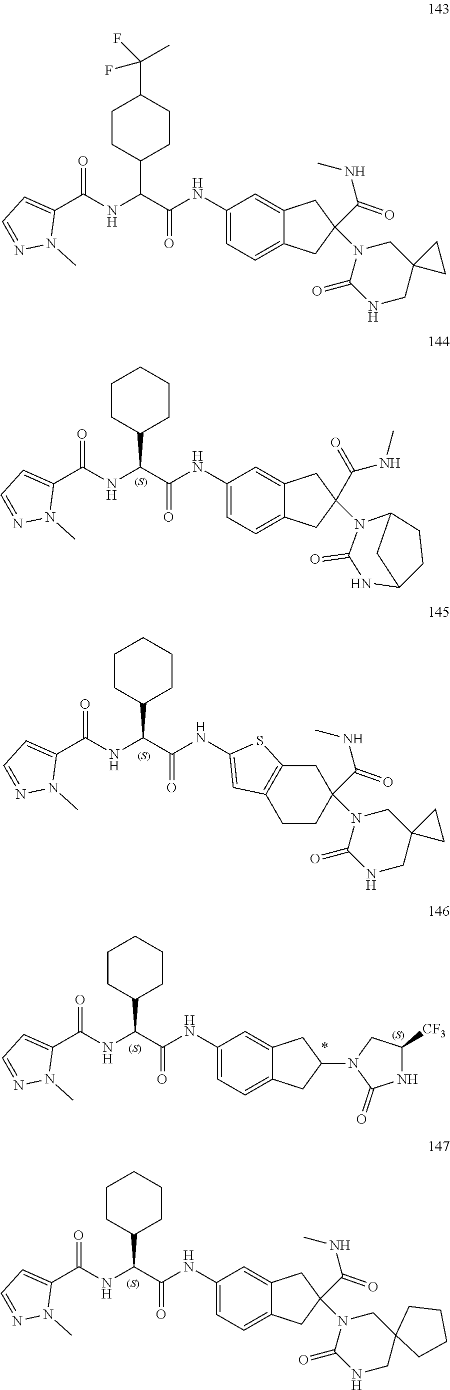

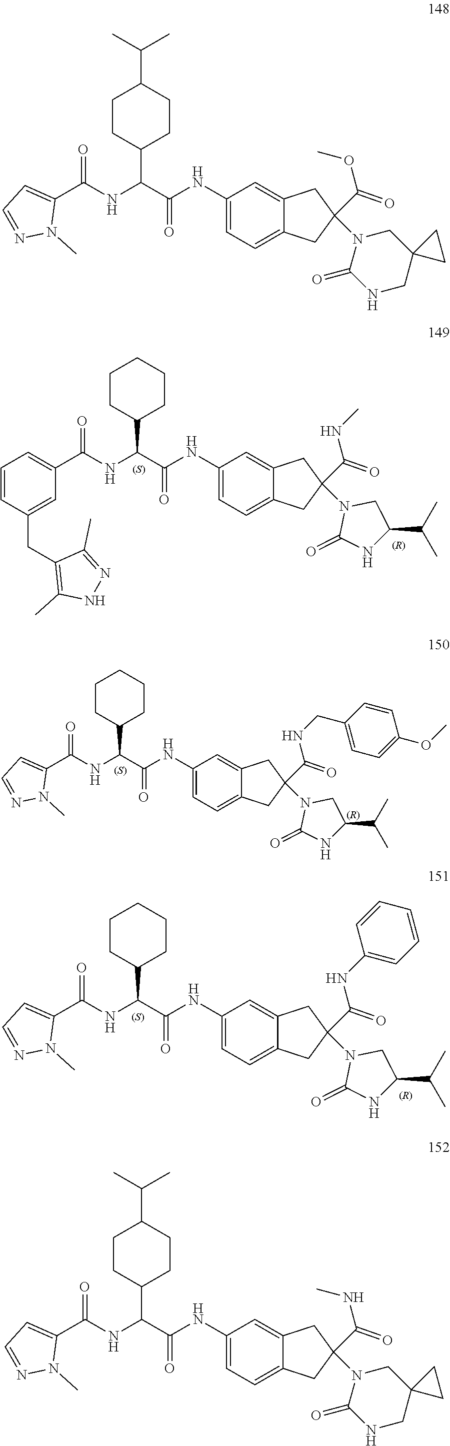

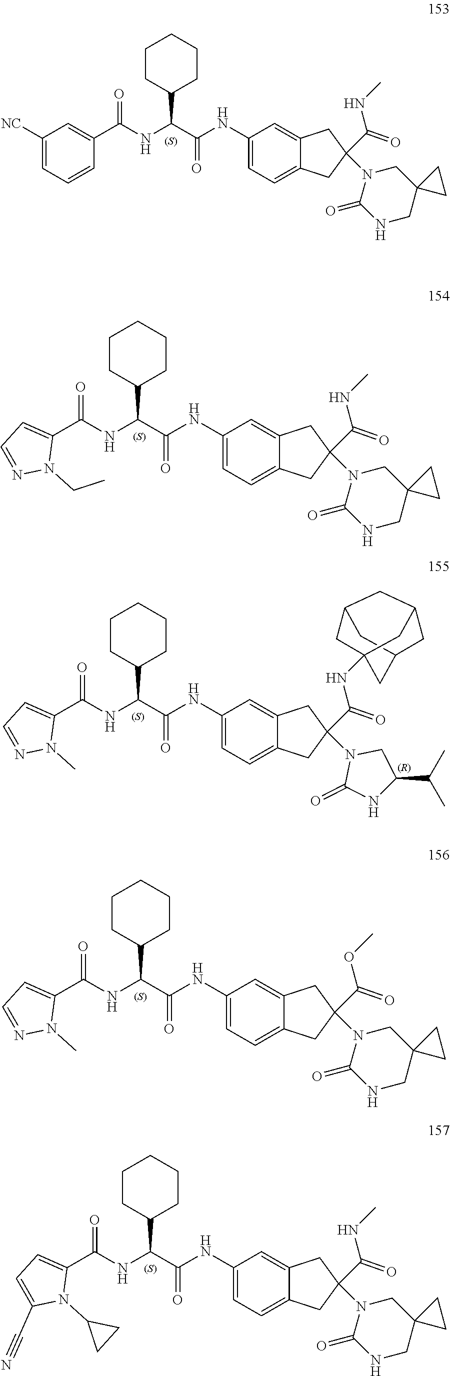

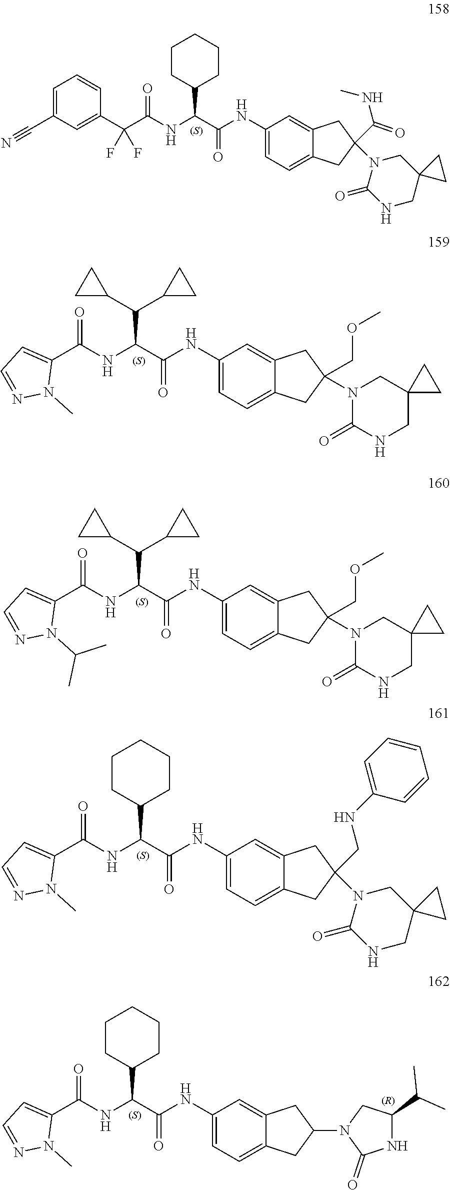

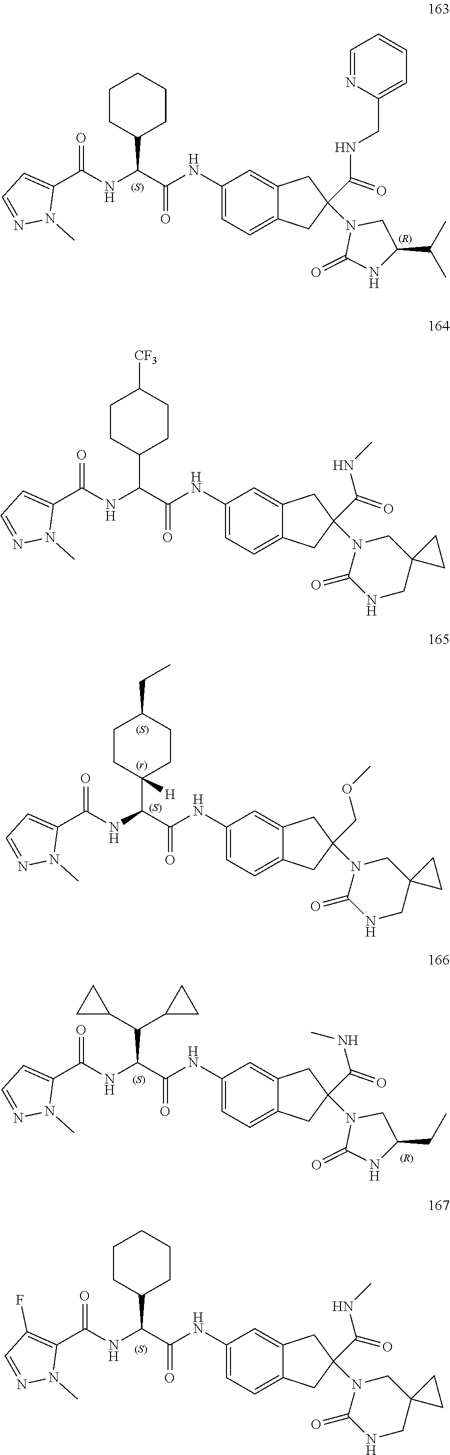

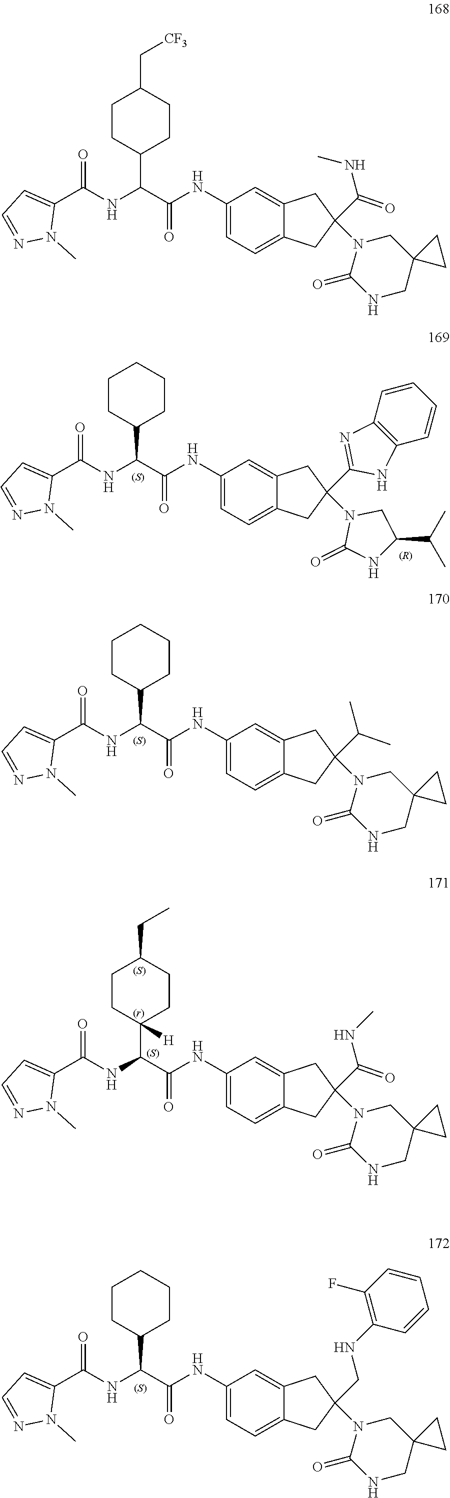

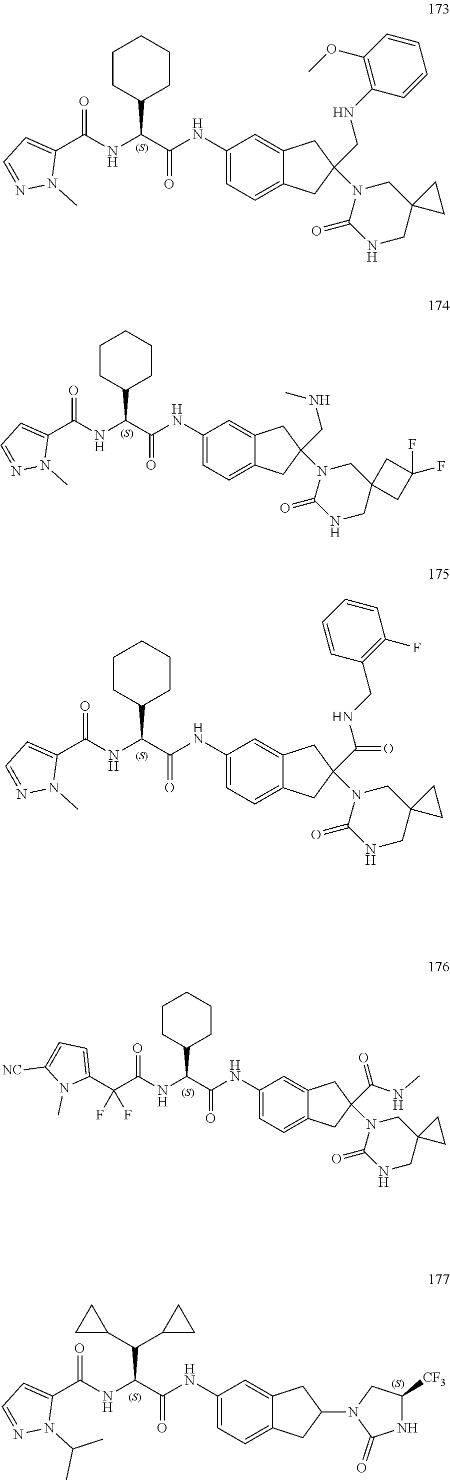

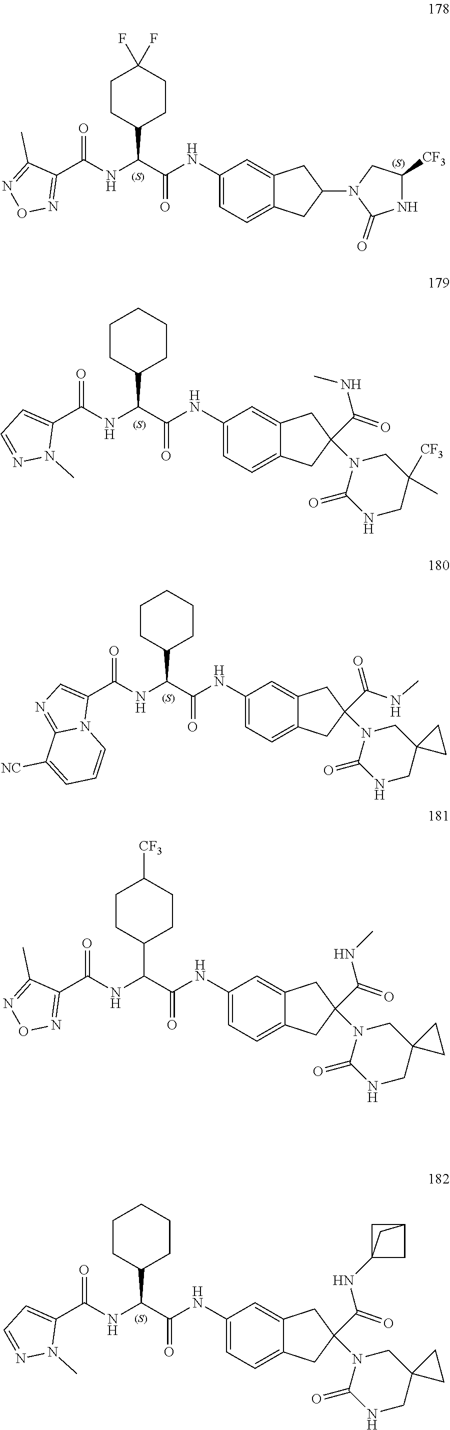

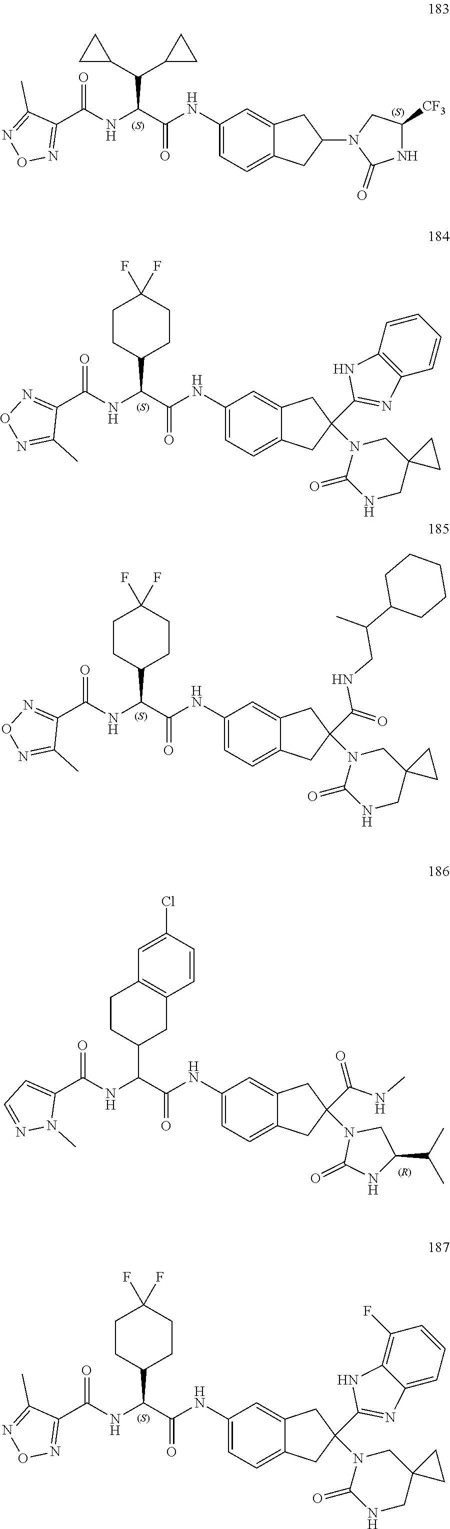

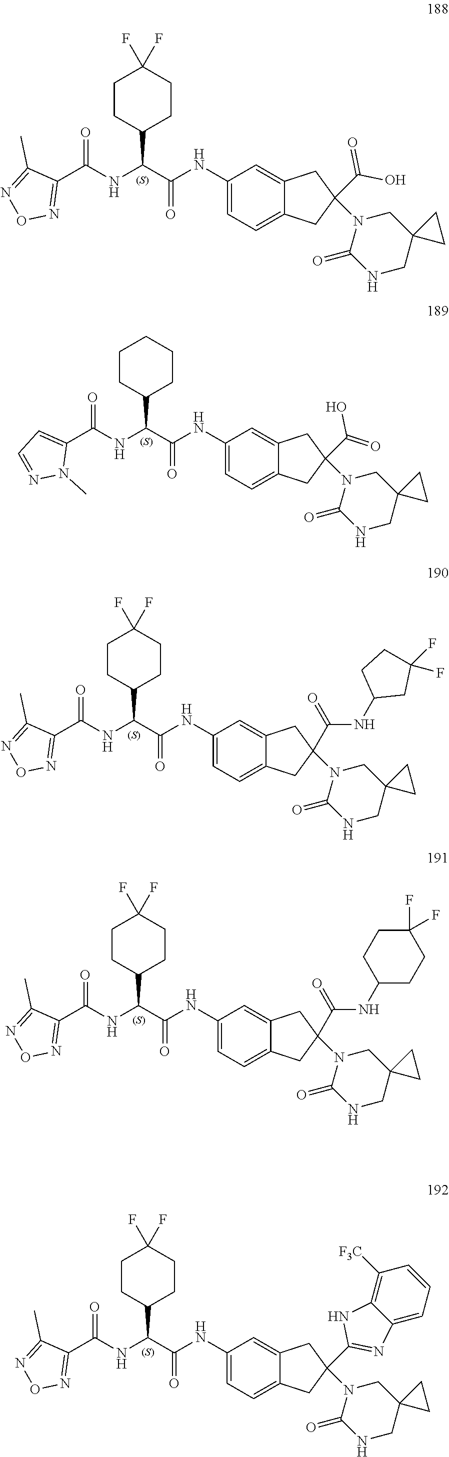

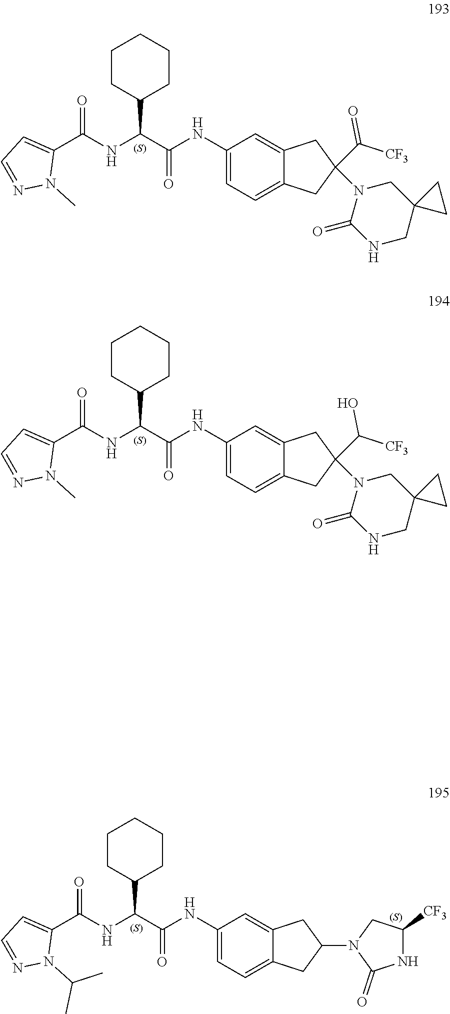

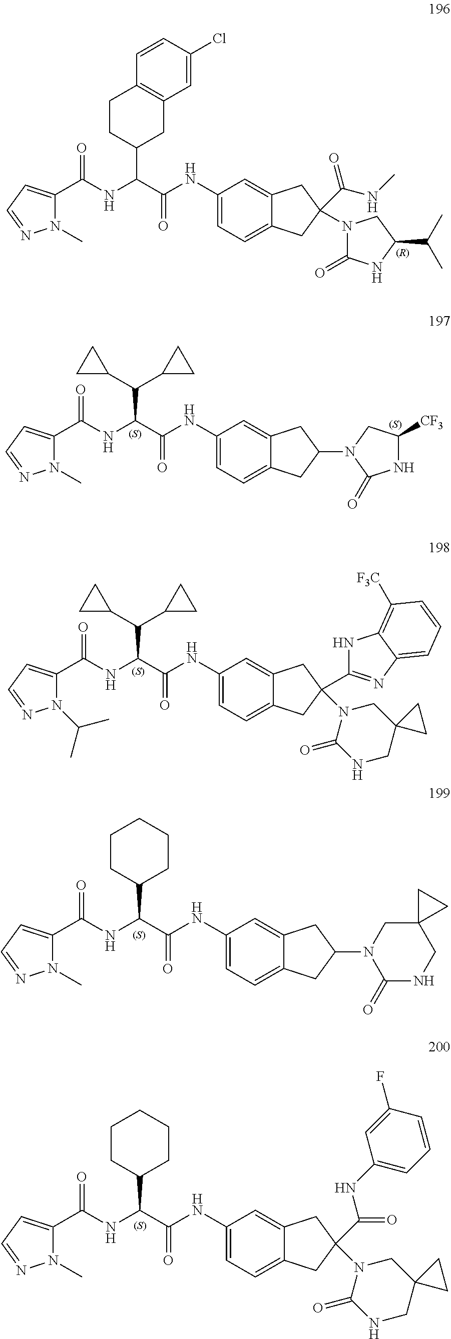

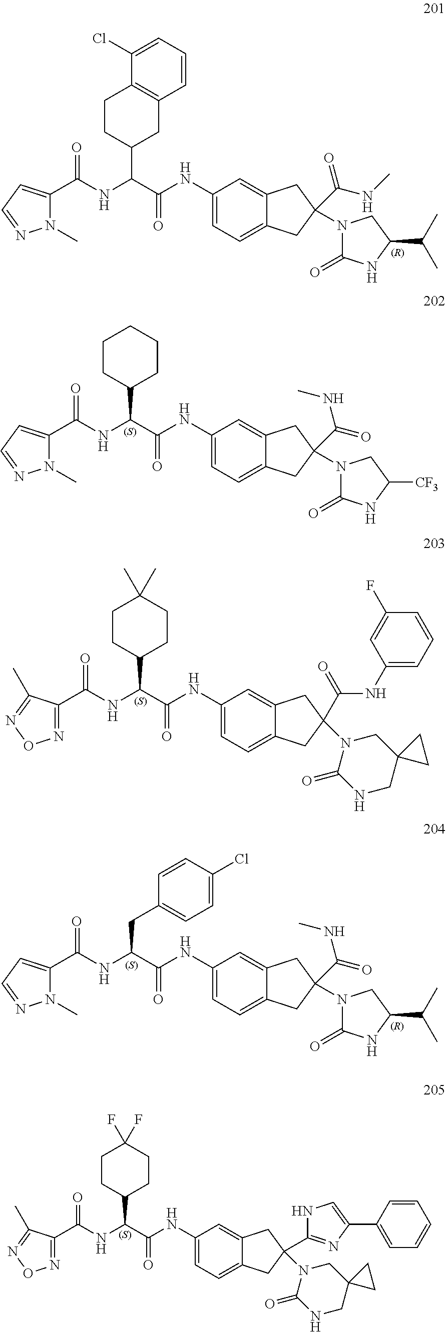

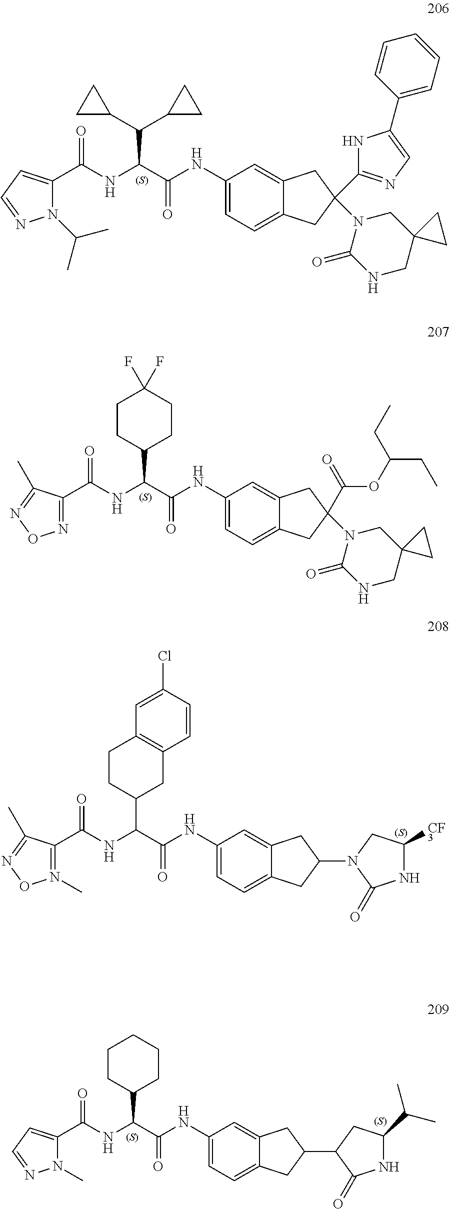

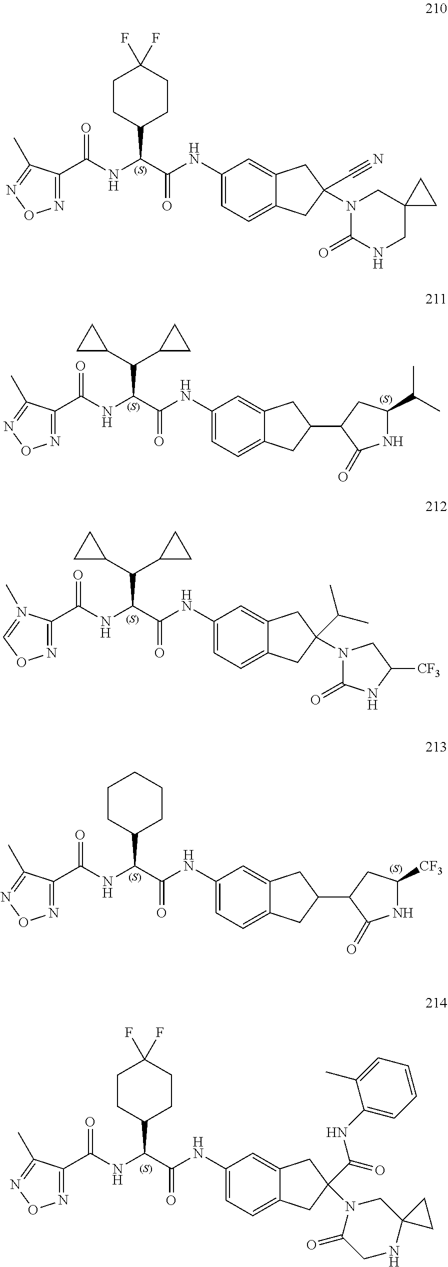

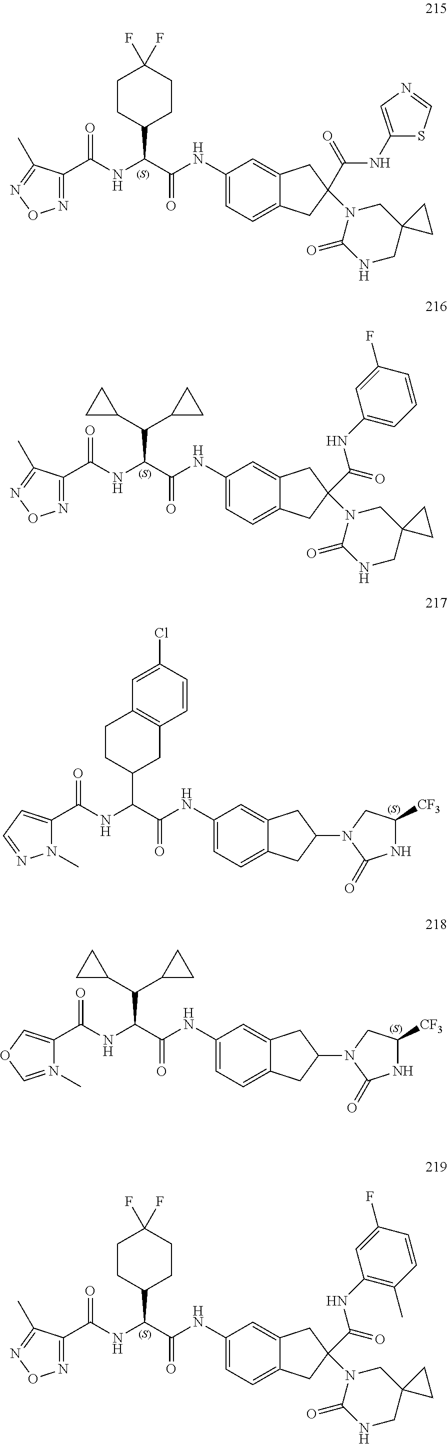

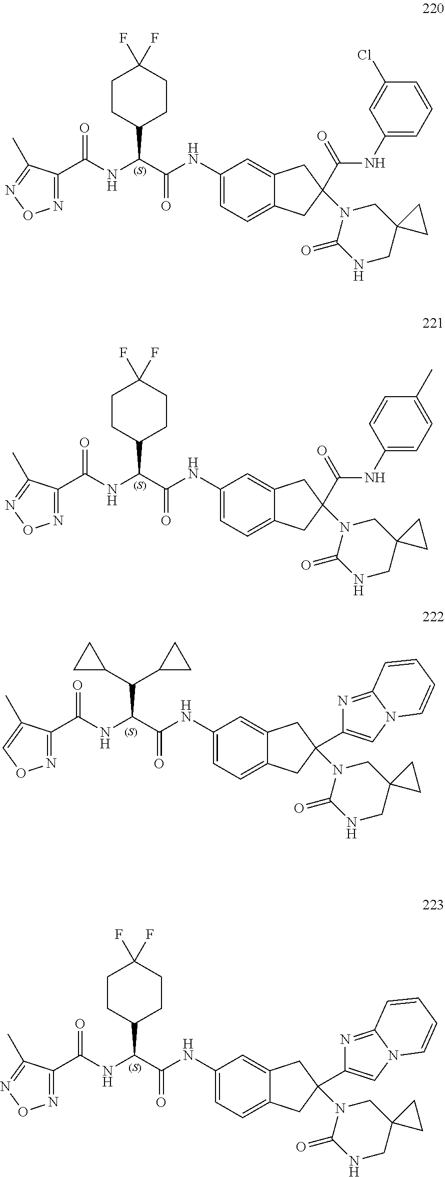

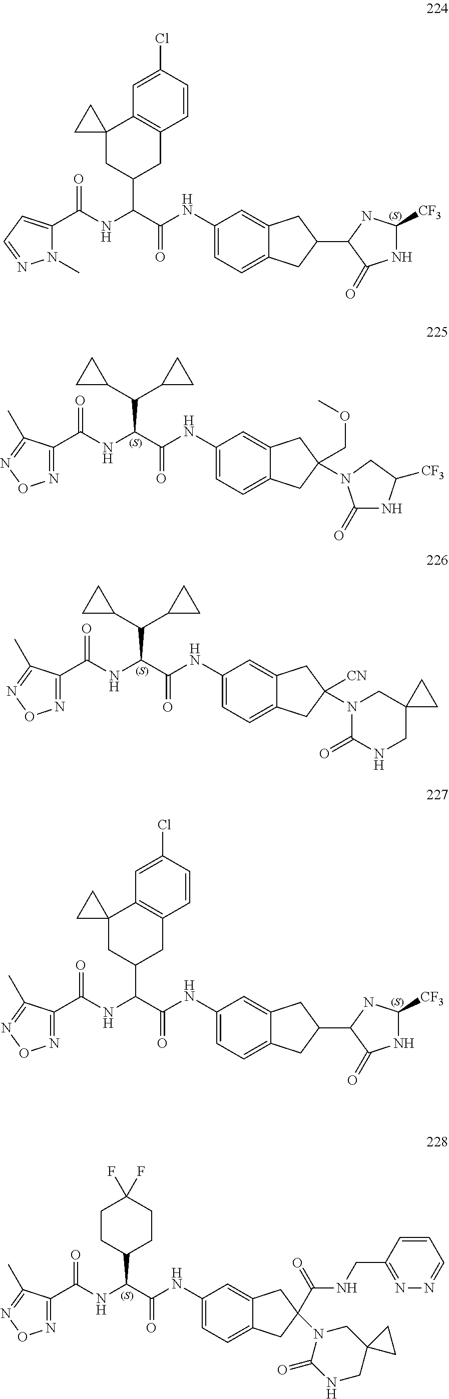

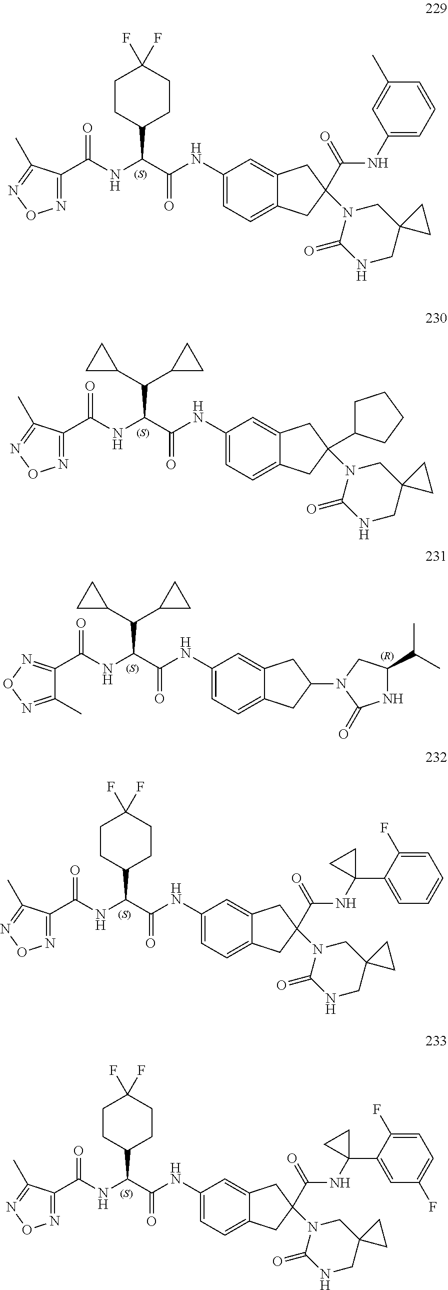

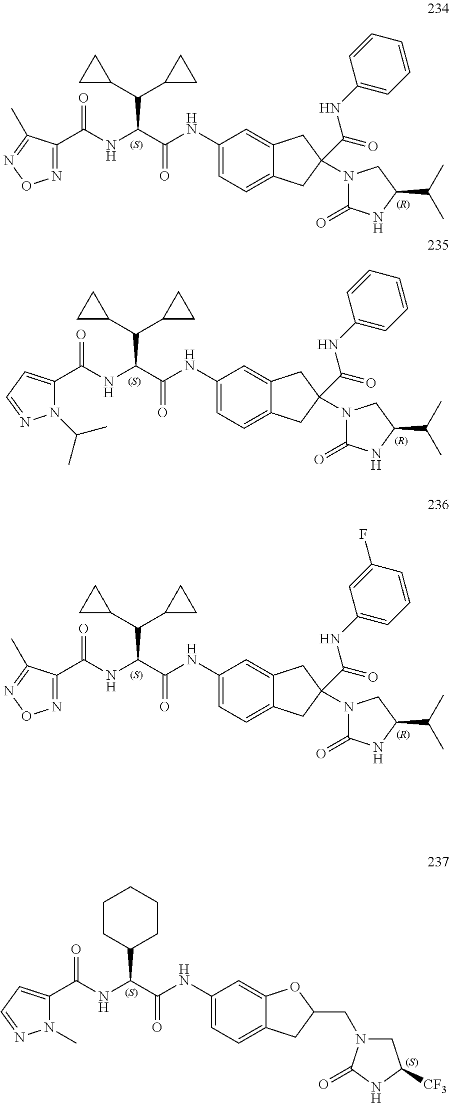

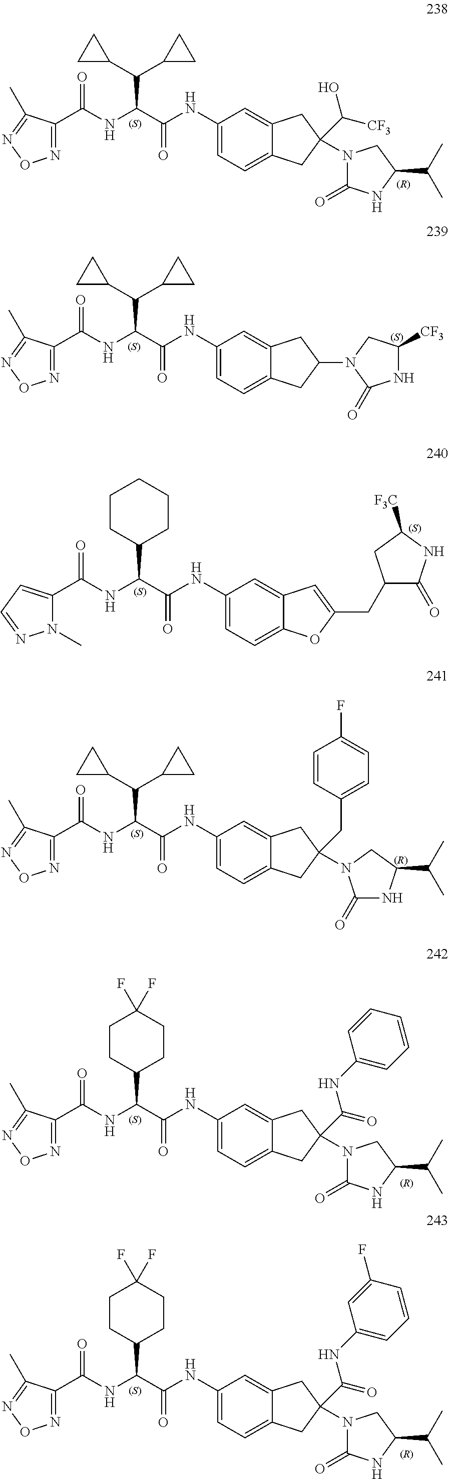

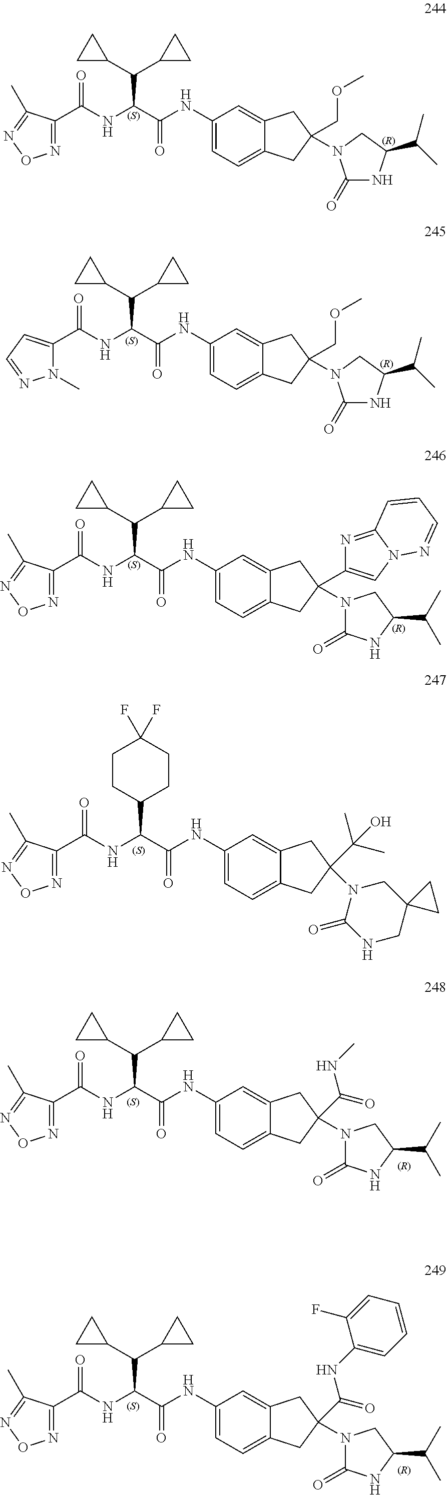

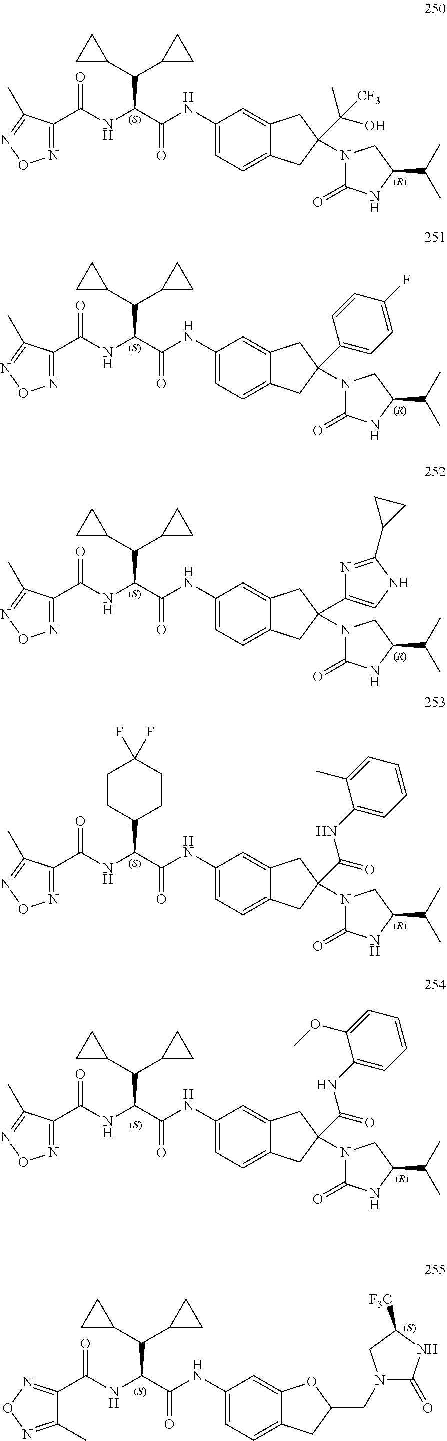

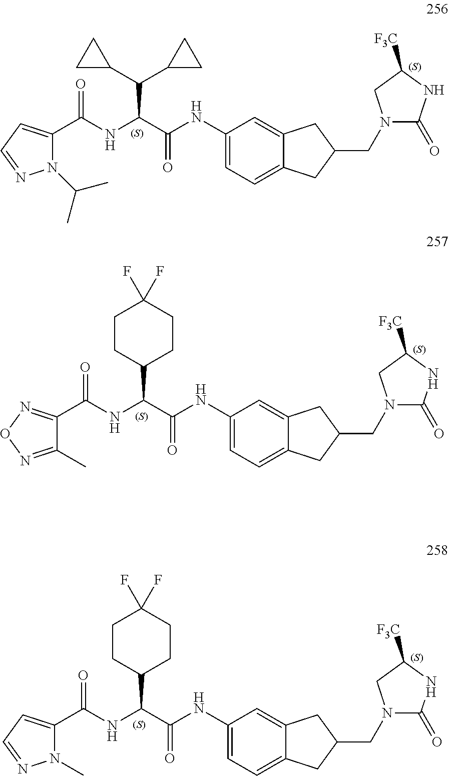

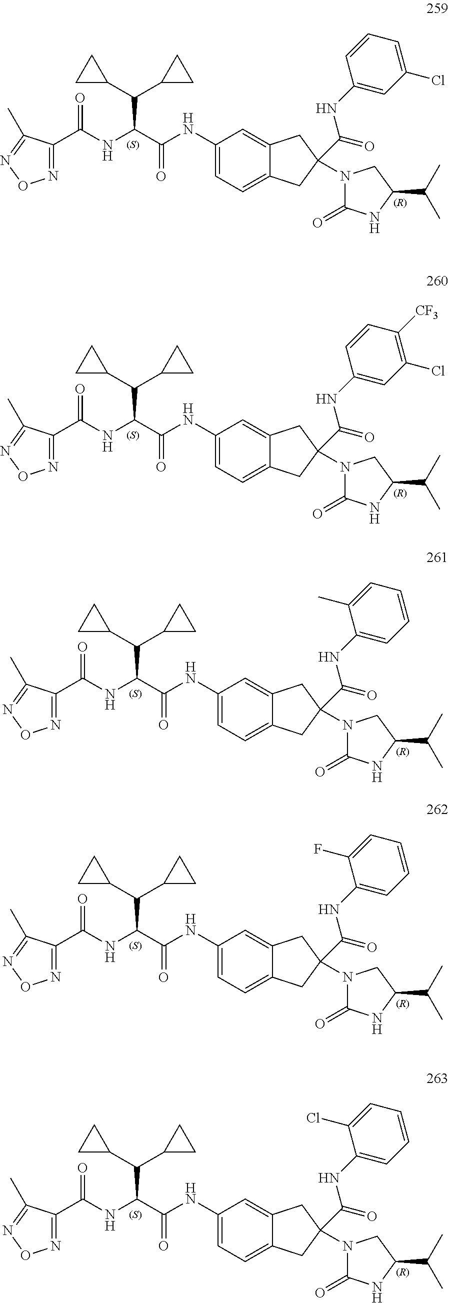

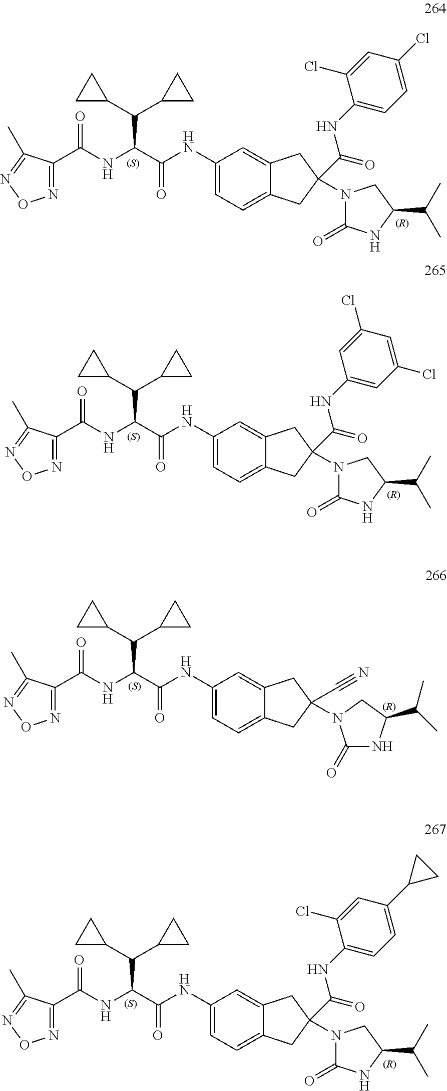

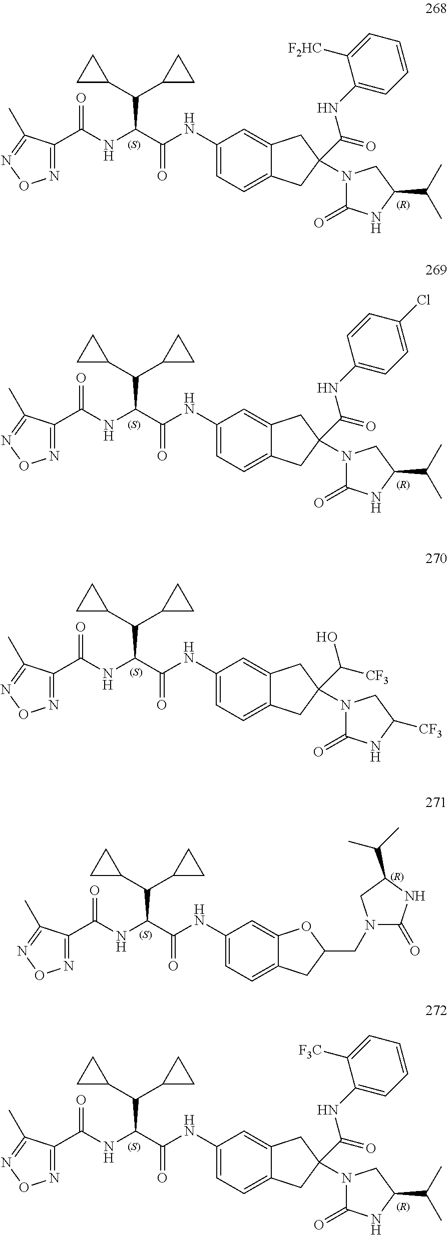

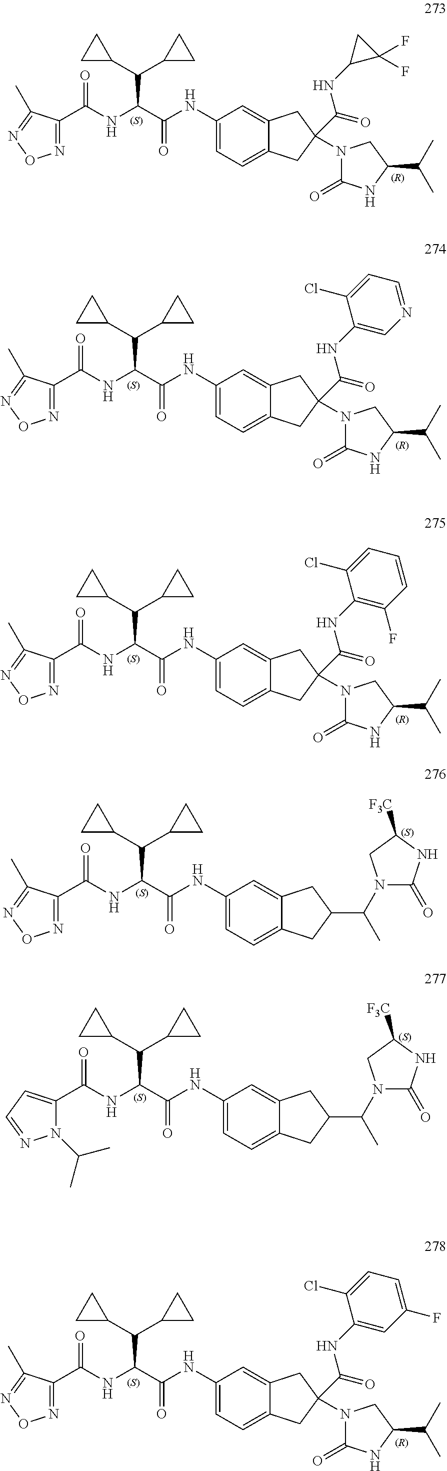

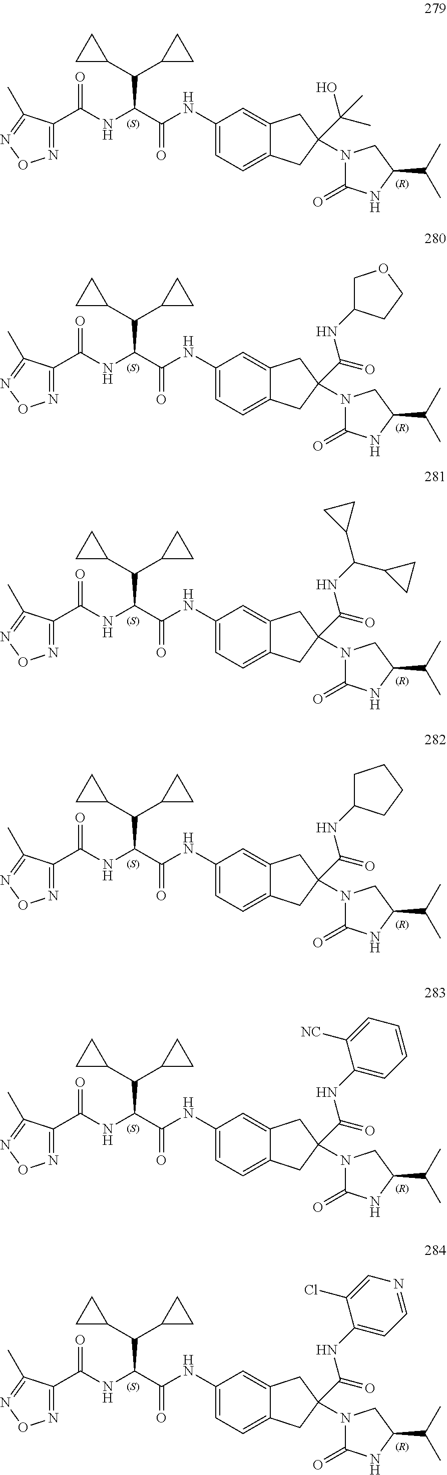

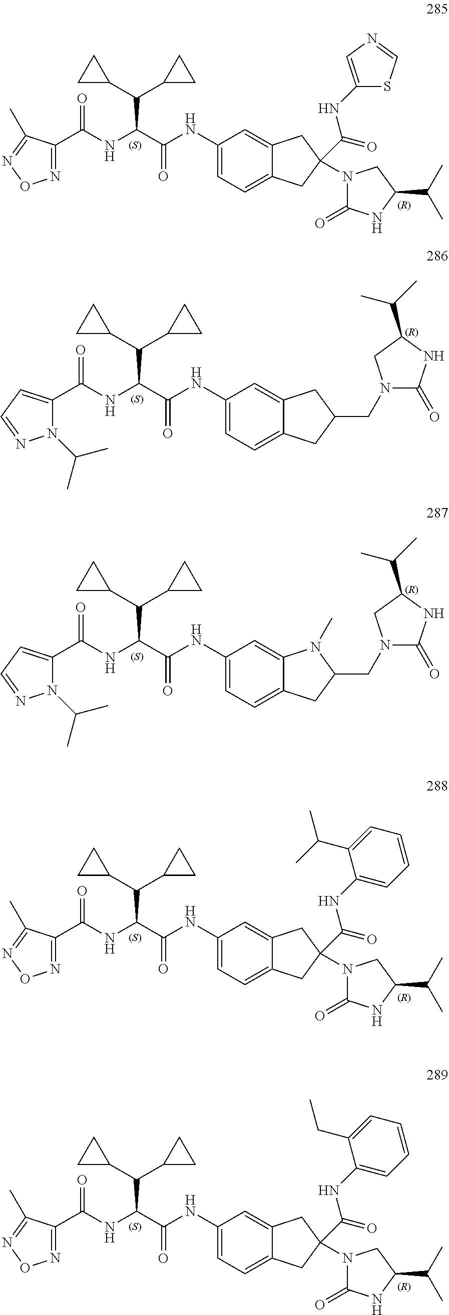

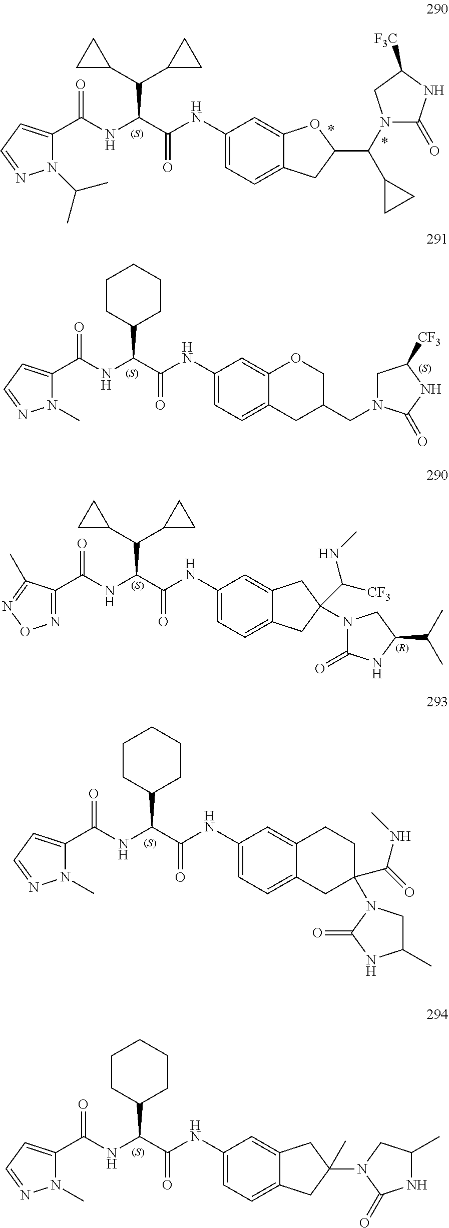

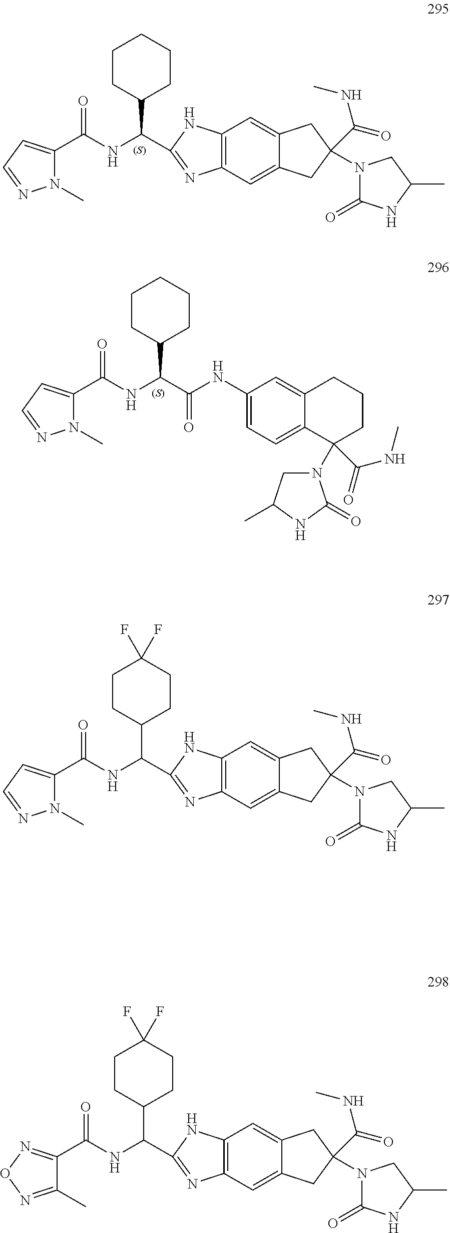

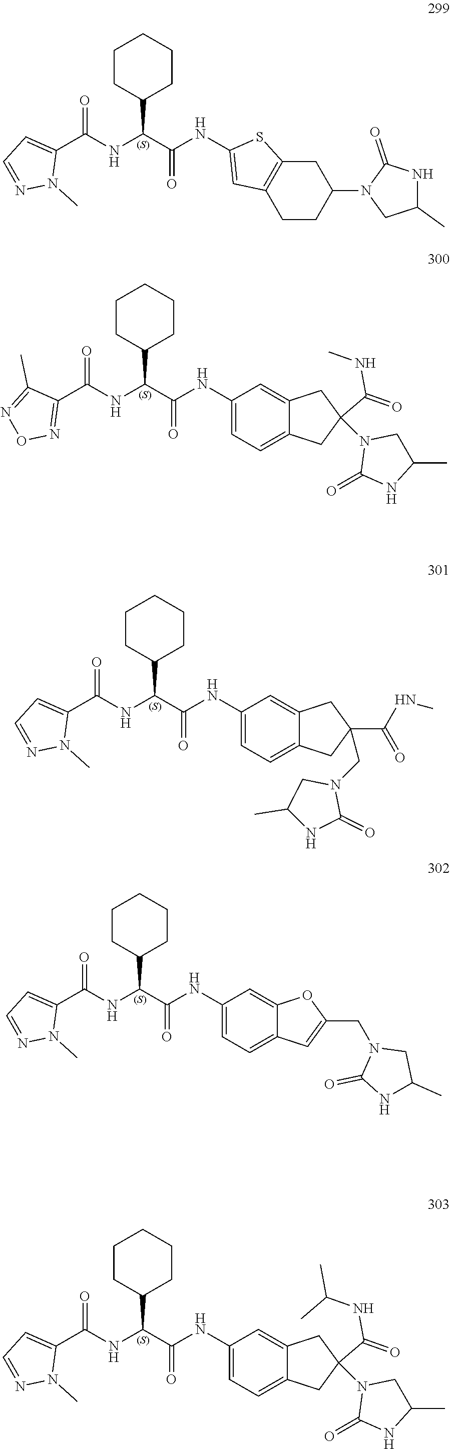

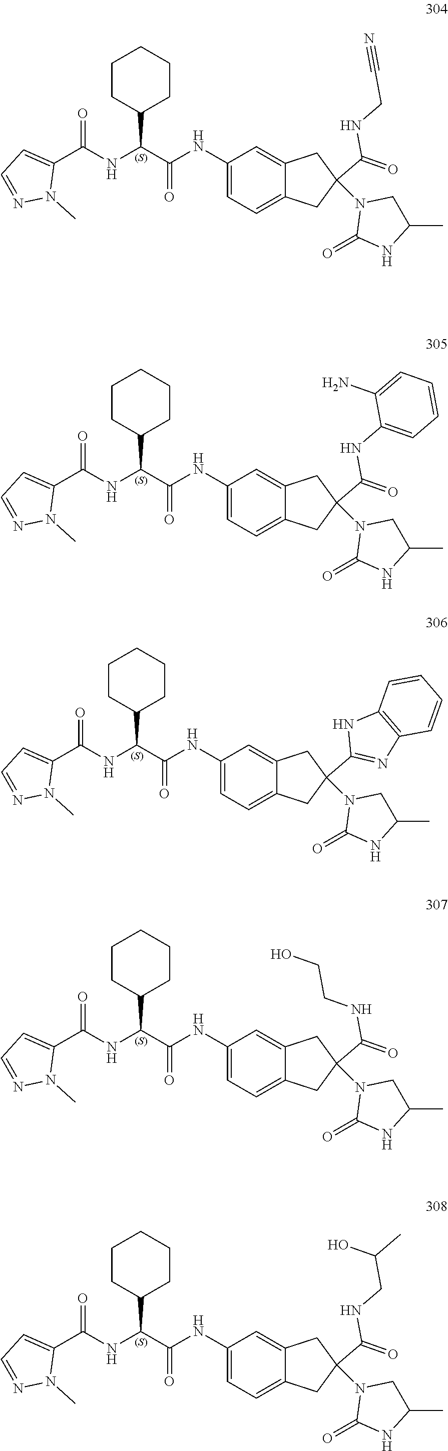

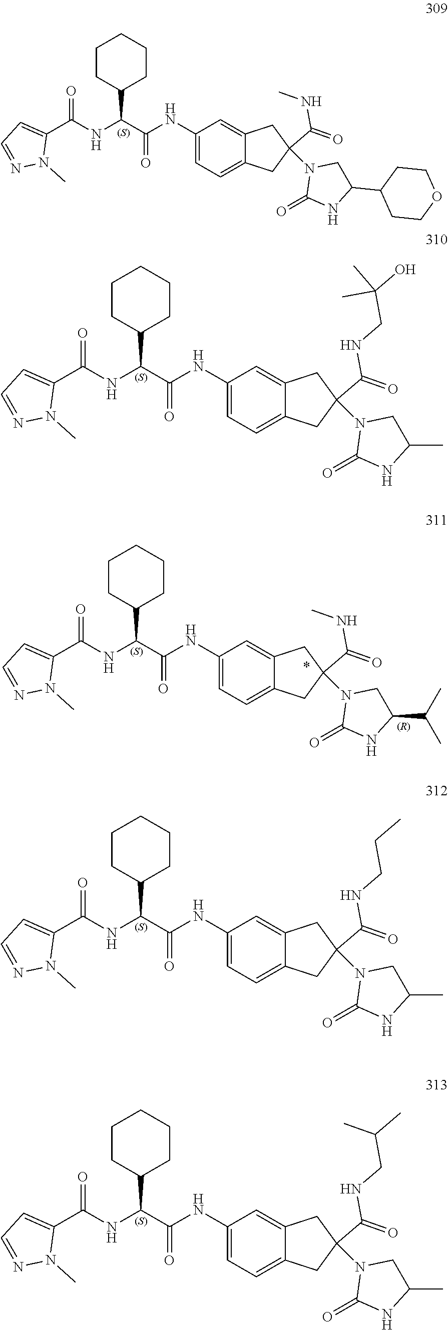

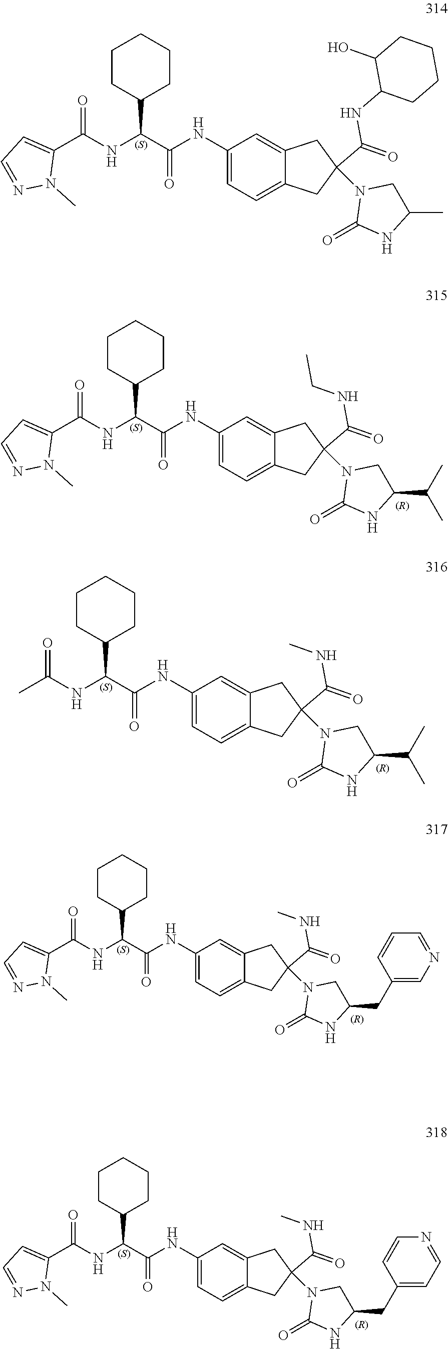

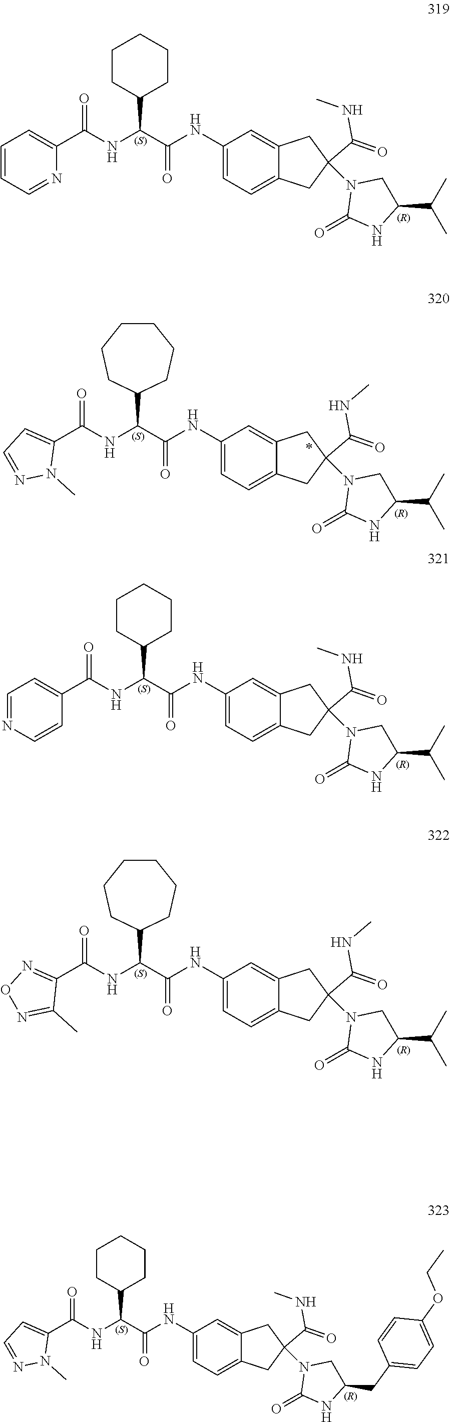

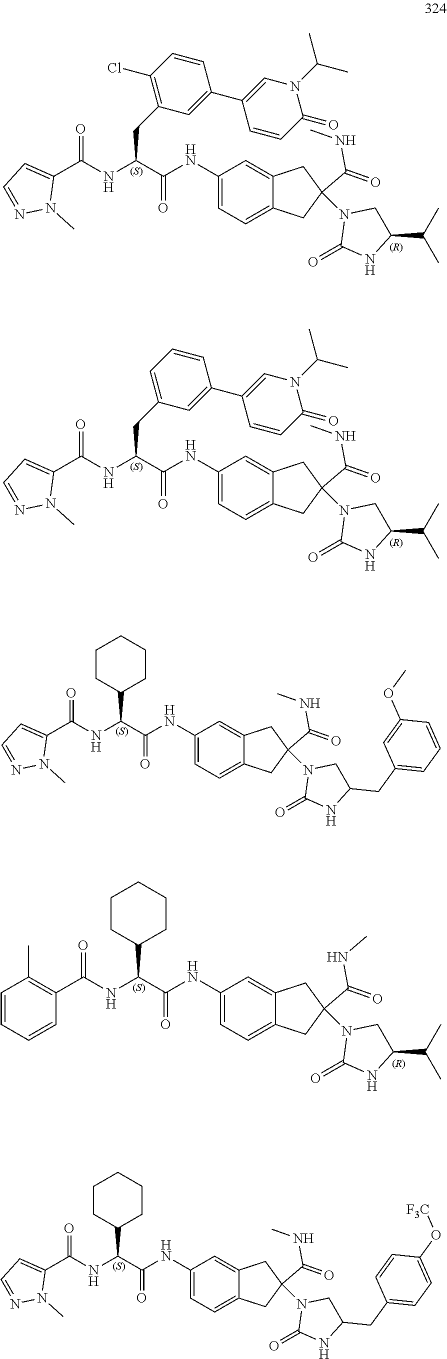

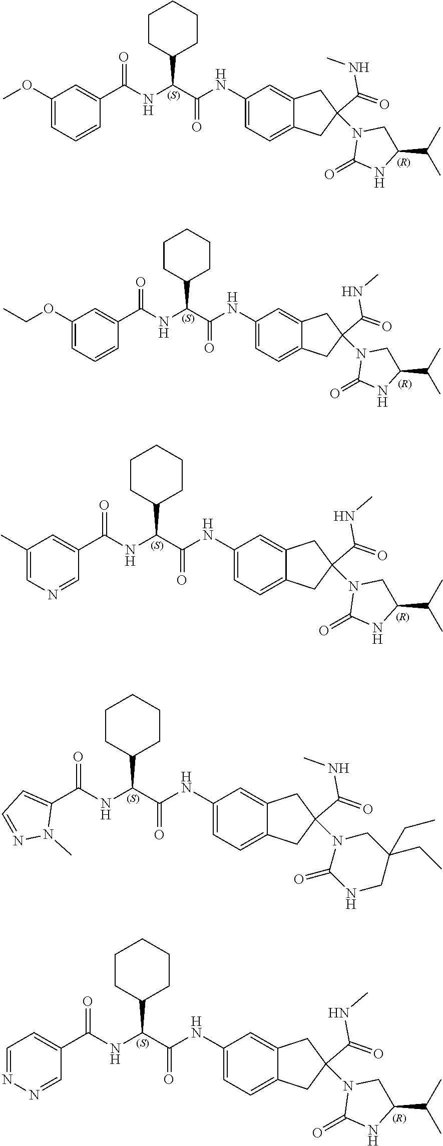

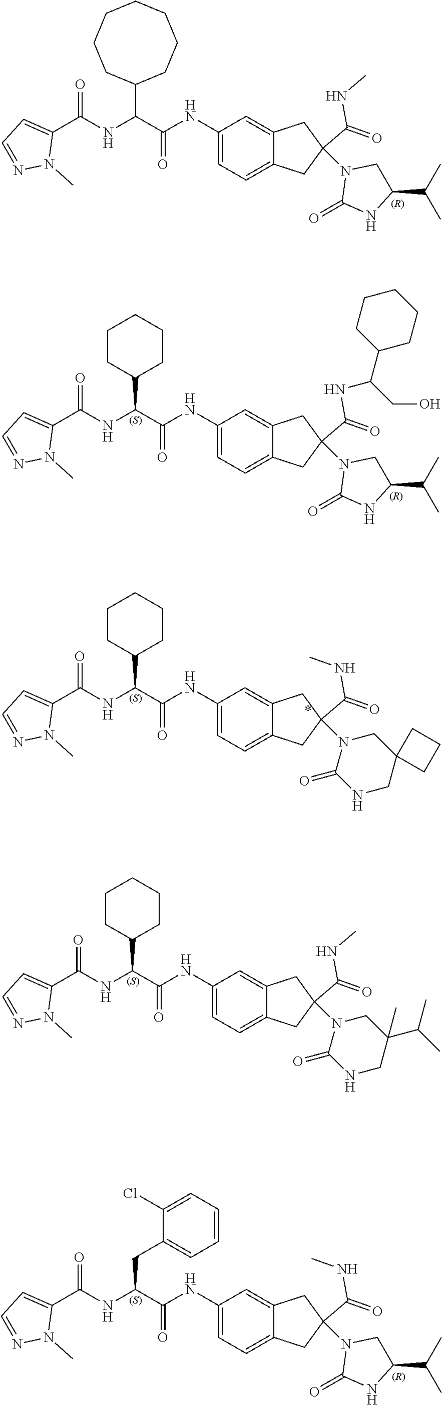

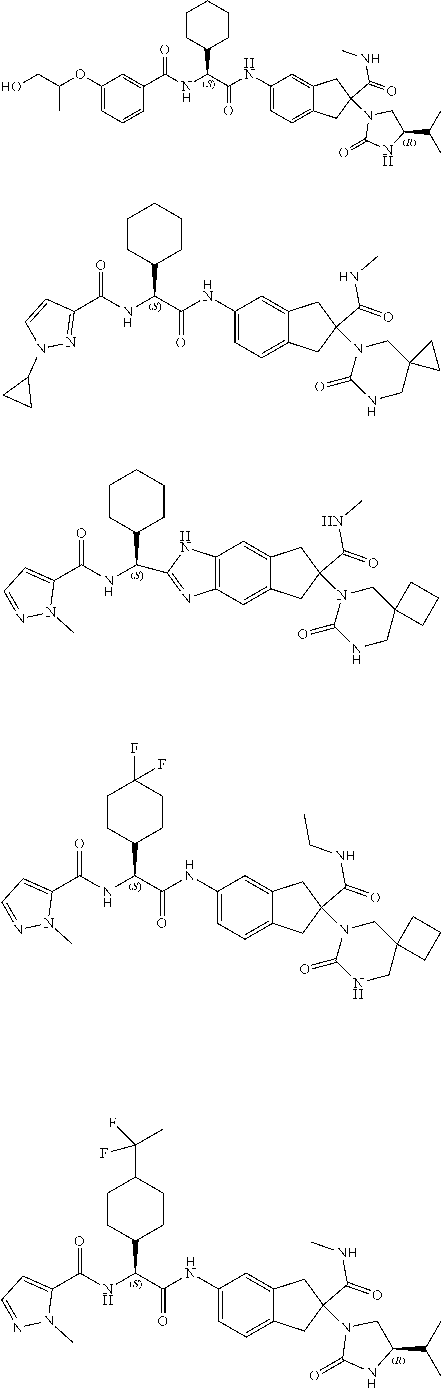

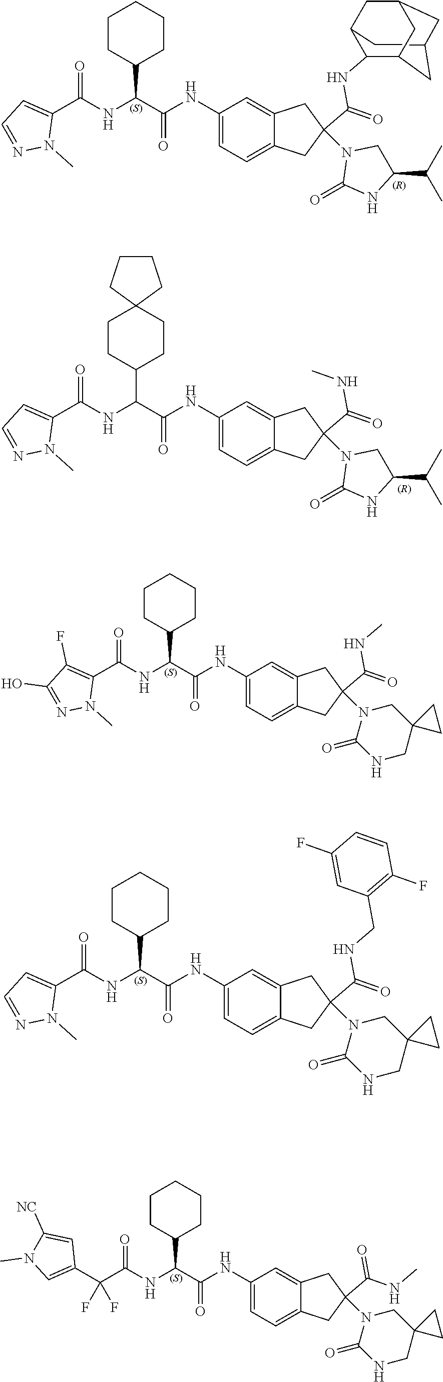

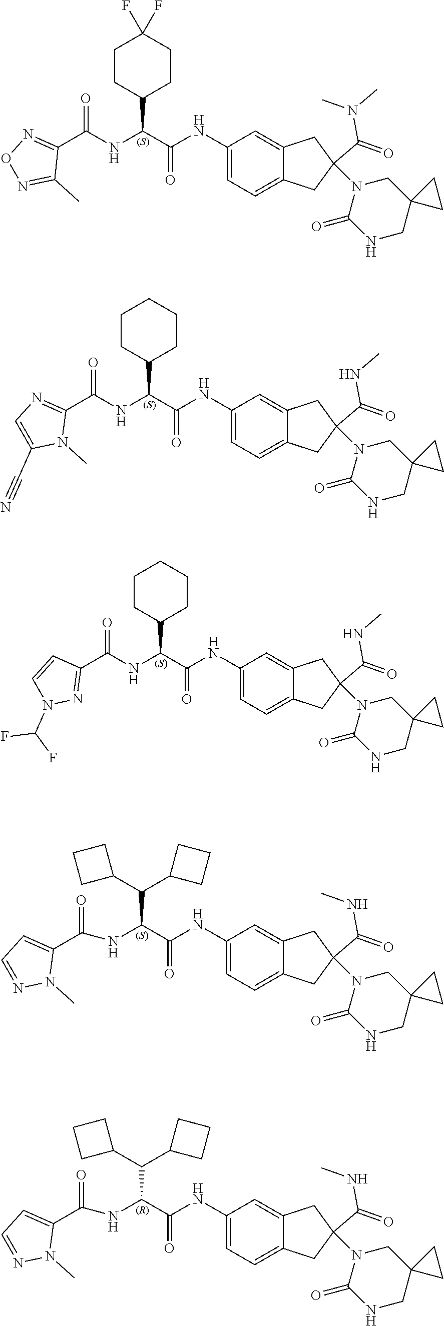

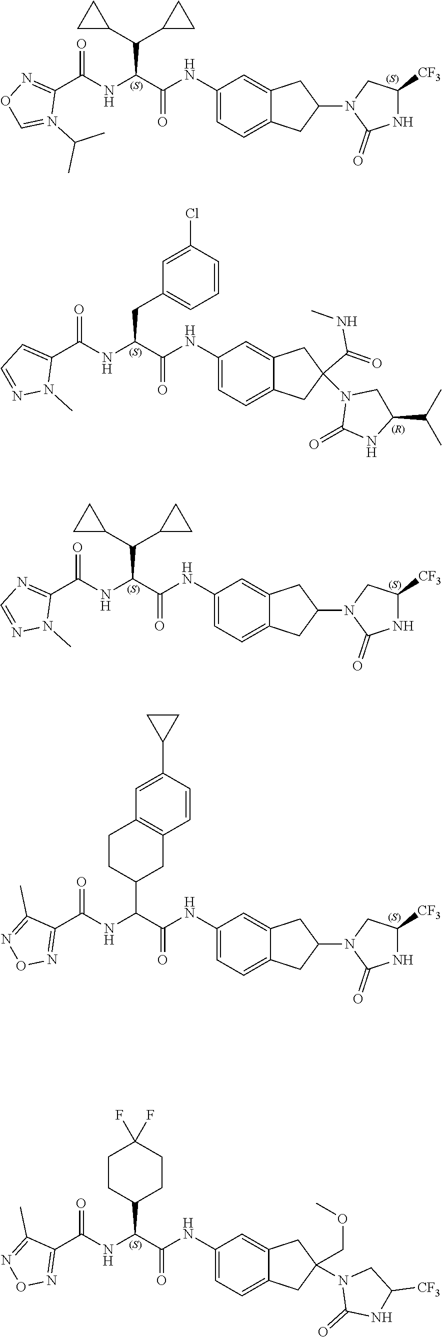

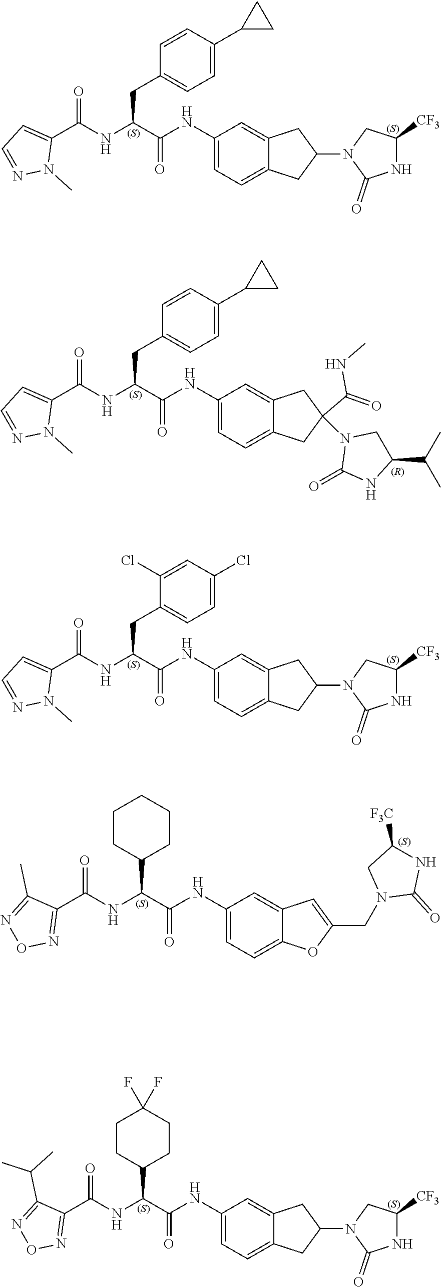

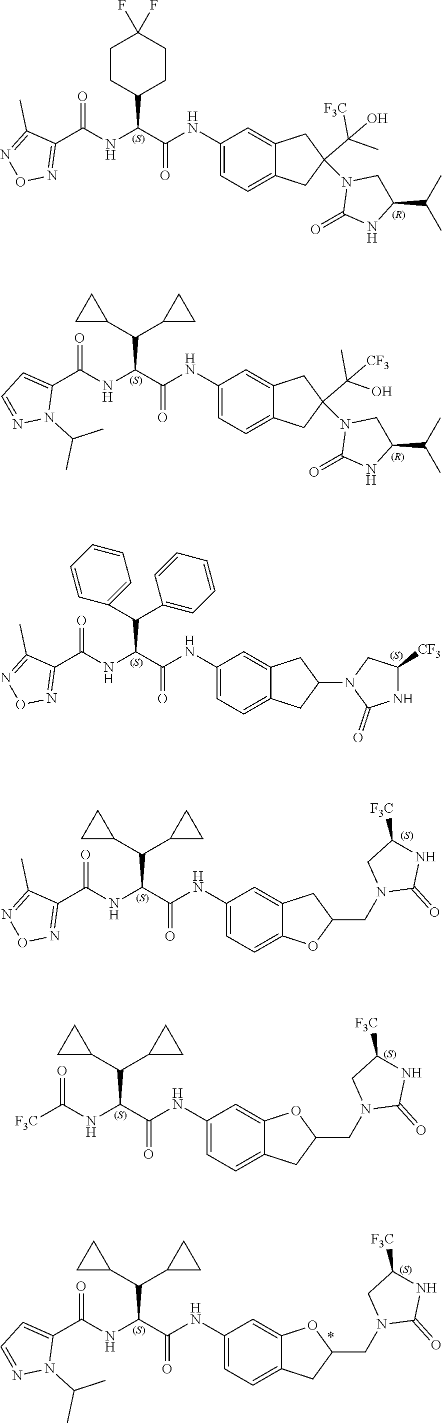

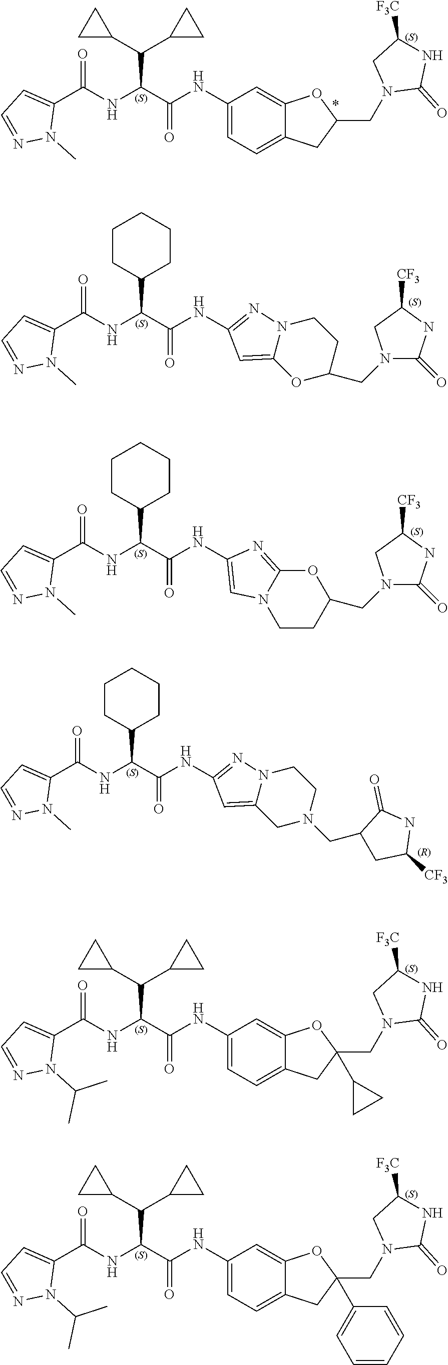

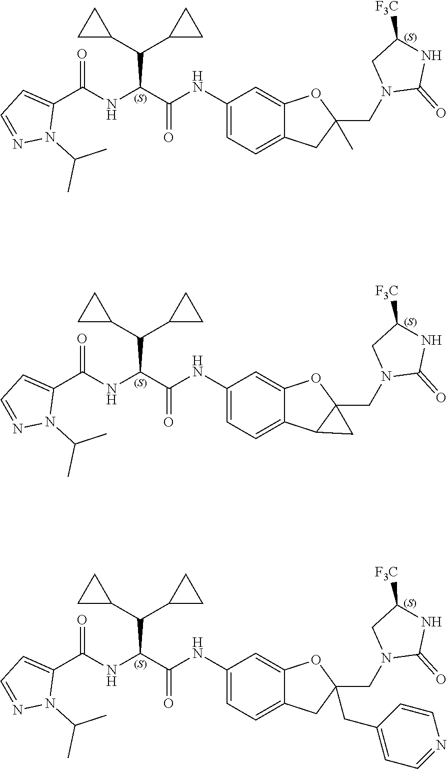

Aspect 33. A pharmaceutical composition comprising a compound of any one of Aspects 1-32 or a pharmaceutically acceptable salt, stereoisomer, tautomer or prodrug thereof, together with a pharmaceutically acceptable excipient.

Aspect 34. A method of decreasing IL-17 activity by inhibition, which comprises administering to an individual the compound according to any one of Aspects 1-32, or a pharmaceutically acceptable salt thereof, including the compound of formula (I) or the specific compounds exemplified herein.

Aspect 35. The method of Aspect 34, wherein the disease is selected from cancer.

Aspect 36. Use of a compound of any one of Aspects 1-32 or a pharmaceutically acceptable salt, stereoisomer, tautomer or prodrug thereof in the preparation of a medicament for treating a disease that can be affected by IL-17.

Aspect 37. The use of Aspect 36, wherein the disease is autoimmune disease or inflammatory disease.

Aspect 38. The use of Aspect 37, wherein the disease is chronic inflammation, psoriasis, spondylarthritis, rheumatoid arthritis or multiple sclerosis.

### DETAILED DESCRIPTION OF THE INVENTION

The following terms have the indicated meanings throughout the specification:

As used herein, including the appended Aspects, the singular forms of words such as “a”, “an” and “the”, include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

The term “aryl” used alone or in combination with other terms refers to a group selected from:
5- and 6-membered carbocyclic aromatic rings, e.g., phenyl;
bicyclic ring systems such as 7- to 12-membered bicyclic ring systems, wherein at least one ring is carbocyclic and aromatic, e.g., naphthyl and indanyl; and,
tricyclic ring systems such as 10- to 15-membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “aryl-alkyl-” refers to an alkyl group as defined above which is further substituted by an aryl group. Examples of an aryl-alkyl group include aryl-C₁₋₈alkyl, such as phenylethyl, or phenylmethyl (benzyl).

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides. The term “C-linked heteroaryl” as used herein means that the heteroaryl group is connected to the core molecule by a bond from a C-atom of the heteroaryl ring

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refers to S, SO or SO₂.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired product of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as the contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be a solution, water solution, oil suspension concentrate, lyophilized powder or the like. Preferably, the formulation of the pharmaceutical composition is selected from a tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cₙ-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### EXAMPLES

The present invention is further exemplified, but not limited to, by the following examples that illustrate the invention.

In the following examples, the abbreviations below are used:

| | |
|---|---|
| AcOH or HOAc | Acetic acid |
| aq. | Aqueous |
| BINAP | (2,2′-bis(diphenylphosphino)-1,1′-binaphthyl) |
| BH₃ | Borane |
| Brine | Saturated aqueous sodium chloride solution |
| Boc₂O | di(tert-butyl) carbonate |
| BSA | Bovine serum albumin |
| CDI | 1,1′-Carbonyldiimidazole |
| DAST | Diethylaminosulfur trifluoride |
| DBN | 1,5-Diazabicyclo[4.3.0]non-5-ene |
| DBU | 1,8-Diazabicyclo[5.4.0]undec-7-ene |
| DCE | 1,2-Dichloroethane |
| DCM | Dichloromethane |
| DMAP | 4-Dimethylaminopyridine |
| CH₃MgBr | Methyl magnesium bromide |
| DIPEA | N,N-Diisopropylethylamine |
| DMF | N,N-Dimethylformamide |
| DMAC | Dimethylacetamide |
| DMSO | Dimethyl sulfoxide |
| DPPA | Diphenylphosphoryl azide |
| EA | Ethyl acetate |
| EDCI | 1-Ethyl-3-(3-dimethylaminopropyl)carbodiimide Hydrochloride |
| EDTA | Ethylenediaminetetraacetic acid |
| EtOH | Ethyl alcohol |
| h or hr | Hour |
| HATU | 1-[Bis(dimethylamino)methylene]-1H-1,2,3-triazolo[4,5- |
| | b]pyridinium 3-oxide hexafluorophosphate |
| Hex | Hexane |
| ¹H NMR | Proton Nuclear Magnetic Resonance |
| H₂O₂ | Hydrogen peroxide |
| HOBt | Hydroxybenzotriazole |
| IPA (i-PrOH) | Isopropyl alcohol |
| KOAc | Potassium Acetate |
| LAH | Lithium aluminum hydride |
| LC-MS | Liquid chromatography-mass spectrometry |
| LDA | Lithium diisopropylamide |
| MeOH | Methanol |
| MsOH | Methanesulfonic acid |
| Min | Minutes |
| n-BuLi | n-Butyllithium |
| NaH | Sodium hydride |
| NaBH(OAc)₃ (STAB) | Sodium triacetoxyborohydride |
| NaBH₃CN | Sodium cyanoborohydride |
| NH₄Cl | Ammonium chloride |
| Pd/C | Palladium on carbon powder |
| Pd(dppf)Cl₂ | [1,1′-Bis(diphenylphosphino)ferrocene]dichloropalladium(II) |
| Pd(PPh₃)₄ | Tetrakis(triphenylphosphine)palladium(0) |
| Pd(OAc)₂ | Palladium acetate |
| Pd(OH)₂/C | Palladium hydroxide on carbon powder |
| PE | Petroleum ether |
| pH | -lg(hydrogen ion concentration) |
| Prep-HPLC | Preparative high-pressure liquid chromatography |
| Prep-MPLC | Preparative medium pressure liquid chromatography |
| Prep-SFC | Preparative supercritical fluid chromatography |
| Pre-TLC | Preparative thin layer chromatography |
| p-TsOH | p-Toluenesulfonic acid |
| r.t. or RT | room temperature |
| sat. | Saturated |
| t-BuOK | Potassium tert-butoxide |
| TBS | tert-butyldimethylsilyl |
| THF | Tetrahydrofuran |
| TEA | Triethylamine |
| TFA | Trifluoroacetic acid |
| TsCl | 4-methylbenzenesulfonyl chloride |
| TMSCN | Trimethylsilyl cyanide |
| TMSCF₃ | (Trifluoromethyl)trimethylsilane |
| TBAF | Tetrabutylammonium fluoride |
| TBSOTf | Trifluoromethanesulfonic acid tert-butyldimethylsilyl ester |
| TiCl₄ | Titanium tetrachloride |
| TIPT | propylphosphonic anhydride solution |
| TMSI | Trimethyliodosilane |

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise.

Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on a Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂0: 4.79 ppm; d₆-DMSO: 2.50 ppm; d6-acetone: 2.05; (CD₃)₂CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LC-MS spectrometer (Agilent 1260) Detector: MWD (190-400 nm), Mass detector: 6120 SQ
Mobile phase: A: acetonitrile with 0.10% Formic acid, B: water with 0.1% Formic acid
Column: Poroshell 120 EC-C18, 4.6×50 mm, 2.7 μm
Gradient method: Flow rate: 1.8 mL/min

| Time (min) | A (%) | B (%) |
|---|---|---|
| 0.00 | 5 | 95 |
| 1.5 | 95 | 5 |
| 2.0 | 95 | 5 |
| 2.1 | 5 | 95 |
| 3.0 | 5 | 95 |

Preparative HPLC was conducted on a column (150×21.2 mm ID, 5 μm, Gemini NX—C18) and (150×19 mm, 5 m, SunFire Prep C18 OBD™) at a different flow rate and injection volume, at room temperature and UV Detection at 214 nm and 254 nm.

Combi Flash was conducted on a column (C18 spherical 20-35 m) at a different flow rate and injection volume, at room temperature and UV Detection at 214 nm and 254 nm.

### Preparation of Intermediates (Int):

### Int A1: 5-((S)-2-amino-2-cyclohexylacetamido)-N-methyl-2-(6-oxo-5,7-diazaspiro[2.5]octan-5-yl)-2,3-dihydro-1H-indene-2-carboxamide

### Step 1: 2-amino-N-methyl-2,3-dihydro-1H-indene-2-carboxamide

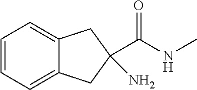

The mixture of methyl 2-amino-2,3-dihydro-1H-indene-2-carboxylate (3.8 g, 20 mmol) in MeNH₂ solution (in EtOH, 33%, wt, 20 mL) was heated to 80° C. in a sealed tube for 4 hrs. The reaction was allowed to cool to ambient temperature and concentrated in vacuum to afford the title compound (3.7 g, yield: 97.32%)

### Step 2: 2-amino-N-methyl-5-nitro-2,3-dihydro-1H-indene-2-carboxamide

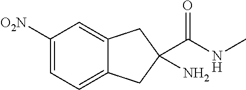 [SMILES: CNC(=O)C1(N)Cc2ccc([N+](=O)[O-])cc2C1]

To the mixture of H₂SO₄ (3 mL) and HNO₃ (3 mL) was added 2-amino-N-methyl-2,3-dihydro-1H-indene-2-carboxamide (3.7 g, 19.46 mmol, a solution in 6 mL HOAc) at 0° C. in batches slowly and stirred for 15 mins. The reaction mixture was poured into NaHCO₃ solution (aq, 200 mL) at 0° C. and then diluted with DCM (200 mL). The organic layer was separated, dried, concentrated in vacuum. The residue was purified by column chromatography on silica gel (eluent: DCM/MeOH (v/v)=20/0 to 20/1) to afford the title compound. (3.28 g, yield: 71.56%).

### Step 3: tert-butyl (2-(methylcarbamoyl)-5-nitro-2,3-dihydro-1H-inden-2-yl)carbamate

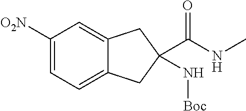 [SMILES: CNC(=O)C1(NC(=O)OC(C)(C)C)Cc2ccc([N+](=O)[O-])cc2C1]

The mixture of 2-amino-N-methyl-5-nitro-2,3-dihydro-1H-indene-2-carboxamide (1.18 g, 5 mmol) and Boc₂O (1.15 g, 5.25 mmol) in DCM (50 mL) was stirred overnight. The mixture was quenched with brine (50 mL), separated and organic phase was dried over Na₂SO₄, concentrated in vacuum. The crude product was purified by column chromatography on silica gel (eluent: DCM/MeOH (v/v)=50/1 to 20/1) to afford the tittle product (1.31 g, yield: 77.52%). MS (ESI, m/e) [M+1]⁺ 336.3.

### Step 4: tert-butyl (5-amino-2-(methylcarbamoyl)-2,3-dihydro-1H-inden-2-yl)carbamate

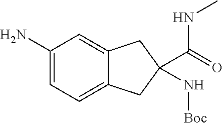 [SMILES: CNC(=O)C1(NC(=O)OC(C)(C)C)Cc2ccc(N)cc2C1]

To the mixture of tert-butyl (2-(methylcarbamoyl)-5-nitro-2,3-dihydro-1H-inden-2-yl)carbamate (1.31 g, 3.90 mmol) and Pd/C (150 mg) in MeOH (50 mL) was bubbled with H₂ balloon and stirred overnight. The mixture was filtrated, concentrated in vacuum, purified by column chromatography on silica gel (eluent: DCM/MeOH (v/v)=50/1 to 20/1) to afford the tittle product (1.06 g, yield: 88.99%). MS (ESI, m/e) [M+1]⁺ 306.0.

### Step 5: tert-butyl (5-((S)-2-(((benzyloxy)carbonyl)amino)-2-cyclohexylacetamido)-2-(methylcarbamoyl)-2,3-dihydro-1H-inden-2-yl)carbamate

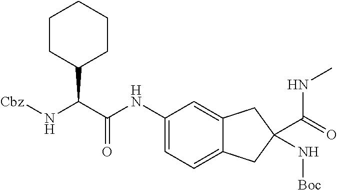 [SMILES: CNC(=O)C1(NC(=O)OC(C)(C)C)Cc2ccc(NC(=O)[C@@H](NC(=O)OCc3ccccc3)C3CCCCC3)cc2C1]

The mixture of (S)-2-(((benzyloxy)carbonyl)amino)-2-cyclohexylacetic acid (909 mg, 3.0 mmol), tert-butyl (5-amino-2-(methylcarbamoyl)-2,3-dihydro-1H-inden-2-yl)carbamate (915 mg, 3.0 mmol), HATU (1.14 g, 3.0 mmol) and TEA (606 mg, 6.0 mmol) in DCM (100 mL) was stirred at room temperature for 4 hrs. The mixture was quenched with aq. NaHCO₃ (150 mL), separated and organic phase was dried over Na₂SO₄, concentrated in vacuo. The crude product was purified by column chromatography on silica gel (eluent: DCM/MeOH (v/v)=50/1 to 20/1) to afford the tittle product (1.41 g, yield: 80.67%). MS (ESI, m/e) [M+1]⁺ 579.3.

### Step 6: benzyl ((1S)-2-((2-amino-2-(methylcarbamoyl)-2,3-dihydro-1H-inden-5-yl)amino)-1-cyclohexyl-2-oxoethyl)carbamate

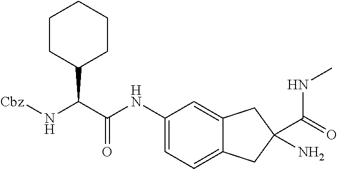 [SMILES: CNC(=O)C1(N)Cc2ccc(NC(=O)[C@@H](NC(=O)OCc3ccccc3)C3CCCCC3)cc2C1]

A mixture of tert-butyl (5-((S)-2-(((benzyloxy)carbonyl)amino)-2-cyclohexylacetamido)-2-(methylcarbamoyl)-2,3-dihydro-1H-inden-2-yl)carbamate (5 g, 8.6 mmol) in DCM (50 mL) and TFA (20 mL) was stirred at 20° C. for 2 hrs. The reaction mixture was then concentrated under reduced pressure. The residue was diluted with H₂O (50 mL) and the aqueous phase was basified by Na₂CO₃ to pH ˜8-9. The mixture was extracted with EtOAc (50 mL×3). The combined organic layers were dried over Na₂SO₄, filtered and concentrated in vacuum to obtain benzyl ((1S)-2-((2-amino-2-(methylcarbamoyl)-2,3-dihydro-1H-inden-5-yl)amino)-1-cyclohexyl-2-oxoethyl)carbamate (4 g, crude). MS (ESI, m/e) [M+H]⁺ 479.3.

### Step 7: benzyl ((1S)-2-((2-(((1-(((tert-butoxycarbonyl)amino)methyl)cyclopropyl)methyl)amino)-2-(methylcarbamoyl)-2,3-dihydro-1H-inden-5-yl)amino)-1-cyclohexyl-2-oxoethyl)carbamate

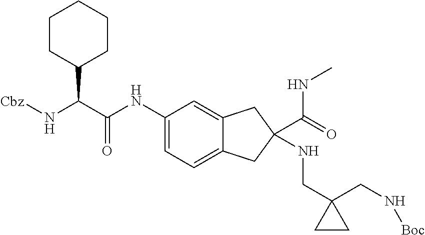 [SMILES: CNC(=O)C1(NCC2(CNC(=O)OC(C)(C)C)CC2)Cc2ccc(NC(=O)[C@@H](NC(=O)OCc3ccccc3)C3CCCCC3)cc2C1]

To a solution of benzyl ((1S)-2-((2-amino-2-(methylcarbamoyl)-2,3-dihydro-1H-inden-5-yl)amino)-1-cyclohexyl-2-oxoethyl)carbamate (0.8 g, 1.7 mmol) and tert-butyl ((1-formylcyclopropyl)methyl)carbamate (0.5 g, 2.5 mmol) in DCM (15 mL) was added AcOH (0.2 g, 3.4 mmol) and NaBH(OAc)3 (0.931 g, 4.2 mmol) at 0° C. The mixture was stirred at 25° C. for 3 hrs. The reaction mixture was then diluted with aq·NaHCO₃ (20 mL) and extracted with DCM (10 mL×3), washed with brine, dried over Na₂SO₄, filtered and concentrated in vacuum. The crude product was purified by column chromatography on silica gel (eluent: Petroleum ether/Ethyl acetate (v/v)=100/1 to 0/1). Title compound (0.65 g, yield: 59%) was obtained.

### Step 8: benzyl ((1S)-2-((2-(((1-(aminomethyl)cyclopropyl)methyl)amino)-2-(methylcarbamoyl)-2,3-dihydro-1H-inden-5-yl)amino)-1-cyclohexyl-2-oxoethyl)carbamate

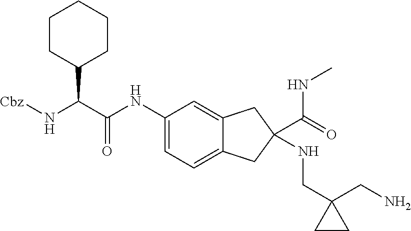 [SMILES: CNC(=O)C1(NCC2(CN)CC2)Cc2ccc(NC(=O)[C@@H](NC(=O)OCc3ccccc3)C3CCCCC3)cc2C1]

A mixture of benzyl ((1S)-2-((2-(((1-(((tert-butoxycarbonyl)amino)methyl)cyclopropyl)methyl)amino)-2-(methylcarbamoyl)-2,3-dihydro-1H-inden-5-yl)amino)-1-cyclohexyl-2-oxoethyl)carbamate (0.65 g, 0.982 mmol) in HCl/EtOAc (10 mL) was stirred at 25° C. for 2 hrs. The reaction mixture was concentrated under reduced pressure to obtain benzyl ((1S)-2-((2-(((1-(aminomethyl)cyclopropyl)methyl)amino)-2-(methylcarbamoyl)-2,3-dihydro-1H-inden-5-yl)amino)-1-cyclohexyl-2-oxoethyl)carbamate (0.55 g, HCl salt). MS (ESI, m/e) [M+H]⁺ 562.3.

### Step 9: benzyl ((1S)-1-cyclohexyl-2-((2-(methylcarbamoyl)-2-(6-oxo-5,7-diazaspiro[2.5]octan-5-yl)-2,3-dihydro-1H-inden-5-yl)amino)-2-oxoethyl)carbamate

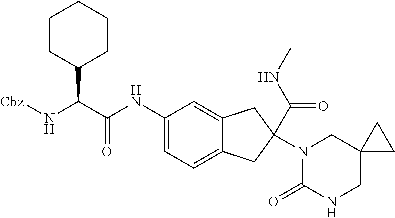 [SMILES: CNC(=O)C1(N2CC3(CC3)CNC2=O)Cc2ccc(NC(=O)[C@@H](NC(=O)OCc3ccccc3)C3CCCCC3)cc2C1]

To a solution of benzyl ((1S)-2-((2-(((1-(aminomethyl)cyclopropyl)methyl)amino)-2-(methylcarbamoyl)-2,3-dihydro-1H-inden-5-yl)amino)-1-cyclohexyl-2-oxoethyl)carbamate (0.55 g, 0.98 mmol) in THF (10 mL) was added TEA (0.198 g, 1.9 mmol) and CDI (0.317 g, 1.9 mmol). The mixture was stirred at 60° C. for 8 hours. The reaction mixture was poured into ice/water (10 mL), extracted with EtOAc (10 mL×3). The combined organic layers were washed with brine, dried over anhydrous Na2SO4, filtered and concentrated in vacuum. The crude product was purified by column chromatography on silica gel (eluent: Petroleum ether/Ethyl acetate (v/v)=100/1 to 0/1) to obtain benzyl ((1S)-1-cyclohexyl-2-((2-(methylcarbamoyl)-2-(6-oxo-5,7-diazaspiro[2.5]octan-5-yl)-2,3-dihydro-1H-inden-5-yl)amino)-2-oxoethyl)carbamate (0.46 g, yield: 80%).

### Step 10: 5-((S)-2-amino-2-cyclohexylacetamido)-N-methyl-2-(6-oxo-5,7-diazaspiro[2.5]octan-5-yl)-2,3-dihydro-1H-indene-2-carboxamide (Int A1)

To a solution of benzyl ((1S)-1-cyclohexyl-2-((2-(methylcarbamoyl)-2-(6-oxo-5,7-diazaspiro[2.5]octan-5-yl)-2,3-dihydro-1H-inden-5-yl)amino)-2-oxoethyl)carbamate (0.41 g, 0.698 mmol) in MeOH (10 mL) and MeNH2·H2O (1 mL) was added Pd(OH)2 (0.1 g, 0.698 mmol). The mixture was stirred at 50° C. for 1 hr under H₂ (15 Psi). The reaction mixture was diluted with water (5 mL) and extracted with EtOAc (5 mL×3). The combined organic layers were washed with brine, dried over anhydrous Na2SO4, filtered and concentrated under reduced pressure to obtain 5-((S)-2-amino-2-cyclohexylacetamido)-N-methyl-2-(6-oxo-5,7-diazaspiro[2.5]octan-5-yl)-2,3-dihydro-1H-indene-2-carboxamide (280 mg, yield: 87%). ¹H NMR (400 MHz, DMSO-d₆) δ ppm: 10.08-9.41 (m, 1H), 7.50 (s, 1H), 7.40-7.22 (m, 2H), 7.06 (d, J=8.4 Hz, 1H), 6.42 (s, 1H), 3.55-3.39 (m, 3H), 3.12-2.93 (m, 5H), 2.91-2.81 (m, 2H), 2.56 (d, J=4.8 Hz, 3H), 2.00-1.81 (m, 1H), 1.69 (d, J=10.0 Hz, 3H), 1.62-1.45 (m, 3H), 1.23-0.97 (m, 5H), 0.45-0.32 (m, 2H), 0.19-0.44 (m, 2H). MS (ESI, m/e) [M+H]⁺ 454.3.

### Int A2: 5-((S)-2-amino-2-cyclohexylacetamido)-2-((R)-4-isopropyl-2-oxoimidazolidin-1-yl)-N-methyl-2,3-dihydro-1H-indene-2-carboxamide

### Step 1: tert-butyl ((2R)-3-methyl-1-((2-(methylcarbamoyl)-5-nitro-2,3-dihydro-1H-inden-2-yl)amino)butan-2-yl)carbamate

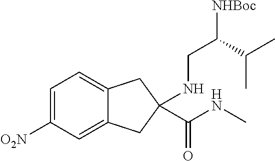 [SMILES: CNC(=O)C1(NC[C@H](NC(=O)OC(C)(C)C)C(C)C)Cc2ccc([N+](=O)[O-])cc2C1]

To a solution of 2-amino-N-methyl-5-nitro-2,3-dihydro-1H-indene-2-carboxamide (10 g, 42.51 mmol) and (R)-tert-butyl (3-methyl-1-oxobutan-2-yl)carbamate (15.4 g, 78.52 mmol) in MeOH (100 mL) was added NaBH₃CN (5.17 g, 858.02 mmol). The mixture was stirred at 25° C. for 12 hrs. The reaction mixture was then concentrated under reduced pressure. The residue was diluted in water (50 mL) and extracted with DCM (50 mL×3). The combined organic layers were dried over anhydrous Na₂SO₄, filtered and concentrated in vacuum. The crude product was purified by prep-MPLC (SiO₂ column, eluent: Petroleum ether/Ethyl acetate (v/v)=10/1 to 0/1) to obtain tert-butyl ((2R)-3-methyl-1-((2-(methylcarbamoyl)-5-nitro-2,3-dihydro-1H-inden-2-yl)amino)butan-2-yl)carbamate (13 g, yield: 73%). ¹H NMR (400 MHz, DMSO-d₆) δ ppm 8.08 (d, J=10.0 Hz, 2H), 7.63-7.61 (m, 1H), 7.34 (d, J=8.0 Hz, 1H), 7.19 (d, J=3.2 Hz, 1H), 4.31 (s, 1H), 3.70-3.59 (m, 3H), 3.44 (s, 1H), 3.02-2.99 (m, 2H), 2.88 (d, J=4.8 Hz, 1H), 2.58-2.55 (m, 1H), 2.35-2.33 (m, 1H), 1.68-1.66 (m, 2H), 1.45 (s, 9H), 0.89-0.85 (m, 6H).

### Step 2: 2-(((R)-2-amino-3-methylbutyl)amino)-N-methyl-5-nitro-2,3-dihydro-1H-indene-2-carboxamide

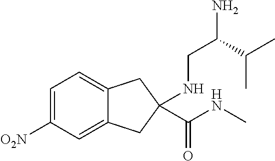 [SMILES: CNC(=O)C1(NC[C@H](N)C(C)C)Cc2ccc([N+](=O)[O-])cc2C1]

To a solution of tert-butyl ((2R)-3-methyl-1-((2-(methylcarbamoyl)-5-nitro-2,3-dihydro-1H-inden-2-yl)amino)butan-2-yl)carbamate (7.8 g, 18.55 mmol) in DCM (80 mL) was added TFA (10 mL). The mixture was stirred at 20° C. for 13 hrs. After concentrated to remove TFA, the reaction mixture was quenched by saturated NaHCO₃ (50 mL) and then extracted with DCM (100 mL×3). The combined organic layers were washed with brine, dried over anhydrous Na₂SO₄, filtered and concentrated in vacuum. The crude product was purified by prep-MPLC (SiO₂ column, eluent: Petroleum ether/Ethyl acetate (v/v)=100/1 to 0/1) to obtain 2-(((R)-2-amino-3-methylbutyl)amino)-N-methyl-5-nitro-2,3-dihydro-1H-indene-2-carboxamide (4.5 g, yield: 76%). MS (ESI, m/e) [M+H]⁺ 321.2.

### Step 3: 2-((R)-4-isopropyl-2-oxoimidazolidin-1-yl)-N-methyl-5-nitro-2,3-dihydro-1H-indene-2-carboxamide

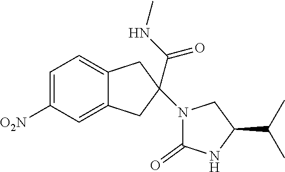 [SMILES: CNC(=O)C1(N2C[C@@H](C(C)C)NC2=O)Cc2ccc([N+](=O)[O-])cc2C1]

A mixture of 2-(((R)-2-amino-3-methylbutyl)amino)-N-methyl-5-nitro-2,3-dihydro-1H-indene-2-carboxamide (4 g, 12.48 mmol), TEA (0.5 mL) and CDI (3.04 g, 18.73 mmol) in THF (40 mL) was stirred at reflux for 1 hour. The reaction mixture was concentrated under reduced pressure and then diluted with water (50 mL) and extracted with DCM (50 mL×3). The combined organic layers were dried over anhydrous Na₂SO₄, filtered and concentrated in vacuum. The crude product was purification by prep-MPLC (SiO₂ column, eluent: Petroleum ether/Ethyl acetate (v/v)=100/1 to 0/1) to obtain 2-((R)-4-isopropyl-2-oxoimidazolidin-1-yl)-N-methyl-5-nitro-2,3-dihydro-1H-indene-2-carboxamide (3.5 g, yield: 80%). ¹H NMR (400 MHz, DMSO-d₆) δ ppm 8.08 (d, J=7.2 Hz, 2H), 7.69 (s, 1H), 7.37-7.33 (m, 1H), 7.12 (s, 1H), 7.05 (s, 1H), 4.75 (s, 1H), 3.87-3.83 (m, 2H), 3.63-3.55 (m, 2H), 3.44-3.39 (m, 2H), 3.14-3.12 (m, 1H), 2.77 (d, J=7.6 Hz, 3H), 1.63-1.62 (m, 1H), 0.89-0.83 (m, 6H).

### Step 4: 5-amino-2-((R)-4-isopropyl-2-oxoimidazolidin-1-yl)-N-methyl-2,3-dihydro-1H-indene-2-carboxamide

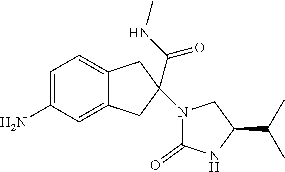 [SMILES: CNC(=O)C1(N2C[C@@H](C(C)C)NC2=O)Cc2ccc(N)cc2C1]

To a solution of 2-((R)-4-isopropyl-2-oxoimidazolidin-1-yl)-N-methyl-5-nitro-2,3-dihydro-1H-indene-2-carboxamide (8.1 g, 25.58 mmol) in CH₃OH (80 mL) was added Pd/C (1 g). The reaction mixture was purged with H₂ for 3 times and hydrogenated under H₂ atmosphere (50 psi) at 50° C. for 12 hrs. The reaction mixture was filtered and the filtrate was concentrated in vacuum. The crude product was purification by prep-MPLC (SiO₂ column, eluent: Petroleum ether/Ethyl acetate (v/v)=100/1 to 0/1) to obtain 5-amino-2-((R)-4-isopropyl-2-oxoimidazolidin-1-yl)-N-methyl-2,3-dihydro-1H-indene-2-carboxamide (6.1 g, yield: 70%). MS (ESI, m/e) [M+H]⁺ 317.3.

### Step 5: tert-butyl ((1S)-1-cyclohexyl-2-((2-((R)-4-isopropyl-2-oxoimidazolidin-1-yl)-2-(methylcarbamoyl)-2,3-dihydro-1H-inden-5-yl)amino)-2-oxoethyl)carbamate

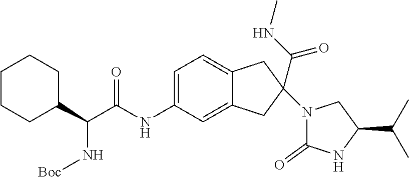 [SMILES: CNC(=O)C1(N2C[C@@H](C(C)C)NC2=O)Cc2ccc(NC(=O)[C@@H](NC(=O)OC(C)(C)C)C3CCCCC3)cc2C1]

To a solution of 5-amino-2-((R)-4-isopropyl-2-oxoimidazolidin-1-yl)-N-methyl-2,3-dihydro-1H-indene-2-carboxamide (1.5 g, 4.74 mmol), TEA (719.59 mg, 7.11 mmol) and (S)-2-((tert-butoxycarbonyl)amino)-2-cyclohexylacetic acid (1.45 g, 5.69 mmol) in DCM (20 mL) was added HATU (2.16 g, 5.69 mmol) at 0° C. The mixture was stirred at 20° C. for 3 hrs. The reaction mixture was then quenched by H₂O (50 mL) and extracted with DCM (50 mL×3). The combined organic layers were dried over anhydrous Na₂SO₄, filtered and concentrated in vacuum. The crude product was purified by prep-MPLC (SiO₂ column, eluent: Petroleum ether/Ethyl acetate (v/v)=100/1 to 1/1) to obtain tert-butyl ((1S)-1-cyclohexyl-2-((2-((R)-4-isopropyl-2-oxoimidazolidin-1-yl)-2-(methylcarbamoyl)-2,3-dihydro-1H-inden-5-yl)amino)-2-oxoethyl)carbamate (2.5 g, yield: 95%). ¹H NMR (400 MHz, DMSO-d₆) δ ppm 9.75 (s, 1H), 7.52-7.45 (m, 2H), 7.34-7.26 (m, 1H), 7.16-7.08 (m, 1H), 6.82-6.75 (m, 2H), 3.92-3.90 (m, 1H), 3.56-3.48 (m, 2H), 3.21-3.18 (m, 2H), 3.11-3.08 (m, 2H), 2.90-2.88 (m, 1H), 2.56 (d, J=4.4 Hz, 3H), 1.75-1.50 (m, 6H), 1.42 (s, 9H), 1.15-1.02 (m, 4H), 0.74-0.65 (m, 6H).

### Step 6: 5-((S)-2-amino-2-cyclohexylacetamido)-2-((R)-4-isopropyl-2-oxoimidazolidin-1-yl)-N-methyl-2,3-dihydro-1H-indene-2-carboxamide (Int A2)

A solution of tert-butyl ((1S)-1-cyclohexyl-2-((2-((R)-4-isopropyl-2-oxoimidazolidin-1-yl)-2-(methylcarbamoyl)-2,3-dihydro-1H-inden-5-yl)amino)-2-oxoethyl)carbamate (2.5 g, 4.5 mmol) in DCM (20 mL) and TFA (5 m) was stirred at 20° C. for 3 hrs. After concentrated in vacuum to remove TFA, the reaction mixture was quenched by saturated NaHCO₃ (50 mL) and extracted with DCM (100 mL×3). The combined organic layers were dried over anhydrous Na₂SO₄, filtered and concentrated in vacuum. The crude product was purified by prep-MPLC (SiO₂ column, eluent: Petroleum ether/Ethyl acetate (v/v)=100/1 to 0/1) to obtain 5-((S)-2-amino-2-cyclohexylacetamido)-2-((R)-4-isopropyl-2-oxoimidazolidin-1-yl)-N-methyl-2,3-dihydro-1H-indene-2-carboxamide (1270 mg, yield: 62%). ¹H NMR (400 MHz, DMSO-d₆) δ ppm 10.25 (s, 1H), 7.55-7.44 (m, 4H), 7.37-7.31 (m, 1H), 7.15 (d, J=8.0 Hz, 1H), 6.77 (d, J=11.2 Hz, 1H), 3.60-3.48 (m, 3H), 3.36-3.34 (m, 3H), 3.22-3.16 (m, 2H), 2.84-2.82 (m, 1H), 2.56 (d, J=4.4 Hz, 3H), 1.75-1.50 (m, 6H), 1.45 (s, 1H), 1.15-1.02 (m, 5H), 0.74-0.67 (m, 6H). MS (ESI, m/e) [M+H]⁺ 456.3.

### Int A3: 5-((S)-2-amino-3,3-dicyclopropylpropanamido)-2-((R)-4-isopropyl-2-oxoimidazolidin-1-yl)-N-(o-tolyl)-2,3-dihydro-1H-indene-2-carboxamide

### Step 1: methyl 5-amino-2-((R)-4-isopropyl-2-oxoimidazolidin-1-yl)-2,3-dihydro-1H-indene-2-carboxylate

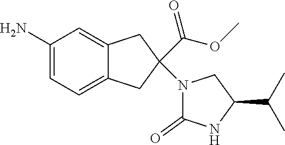 [SMILES: COC(=O)C1(N2C[C@@H](C(C)C)NC2=O)Cc2ccc(N)cc2C1]

To a solution of methyl 2-((R)-4-isopropyl-2-oxoimidazolidin-1-yl)-5-nitro-2,3-dihydro-1H-indene-2-carboxylate (5.9 g, 16.9 mmol, prepared by the procedure similar with 2-((R)-4-isopropyl-2-oxoimidazolidin-1-yl)-N-methyl-5-nitro-2,3-dihydro-1H-indene-2-carboxamide as described in Int A2) in MeOH (60 mL) was added Raney Ni (0.6 g, 1.69 mmol) at r.t. under N₂. The mixture was hydrogenated under H₂ atmosphere (15 psi) at 60° C. for 1 hour. The reaction mixture was filtered through a celite pad and washed with MeOH (50 mL). The filtrate was concentrated in vacuum to obtain methyl 5-amino-2-((R)-4-isopropyl-2-oxoimidazolidin-1-yl)-2,3-dihydro-1H-indene-2-carboxylate (2.5 g, crude), which was used directly for next step. MS (ESI, m/e) [M+H]⁺ 318.1.

### Step 2: methyl 5-((S)-2-(((benzyloxy)carbonyl)amino)-3,3-dicyclopropylpropanamido)-2-((R)-4-isopropyl-2-oxoimidazolidin-1-yl)-2,3-dihydro-1H-indene-2-carboxylate

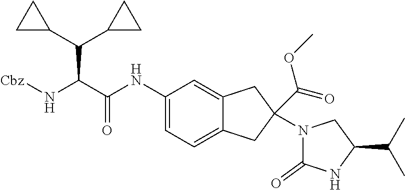 [SMILES: COC(=O)C1(N2C[C@@H](C(C)C)NC2=O)Cc2ccc(NC(=O)[C@@H](NC(=O)OCc3ccccc3)C(C3CC3)C3CC3)cc2C1]

To a solution of methyl 5-amino-2-((R)-4-isopropyl-2-oxoimidazolidin-1-yl)-2,3-dihydro-1H-indene-2-carboxylate (2.5 g), DIEA (3.05 g, 23.6 mmol) and (S)-2-(((benzyloxy)carbonyl)amino)-3,3-dicyclopropylpropanoic acid (2.39 g, 7.88 mmol) in DCM (25 mL) was added HATU (3.0 g, 7.88 mmol) at 0° C. The mixture was stirred at r.t. for 12 hrs. The reaction mixture was diluted with water (30 mL) and then extracted with DCM (30 mL×3). The combined organic layers were washed with brine, dried over anhydrous Na₂SO₄, filtered and concentrated in vacuum. The crude product was purified by column chromatography on silica gel(eluent: Petroleum ether/Ethyl acetate (v/v)=100/1 to 0/1) to obtain methyl 5-((S)-2-(((benzyloxy)carbonyl)amino)-3,3-dicyclopropylpropanamido)-2-((R)-4-isopropyl-2-oxoimidazolidin-1-yl)-2,3-dihydro-1H-indene-2-carboxylate (2.5 g, yield: 53%). MS (ESI, m/e) [M+H]⁺ 603.3.

### Step 3: 5-((S)-2-(((benzyloxy)carbonyl)amino)-3,3-dicyclopropylpropanamido)-2-((R)-4-isopropyl-2-oxoimidazolidin-1-yl)-2,3-dihydro-1H-indene-2-carboxylic acid

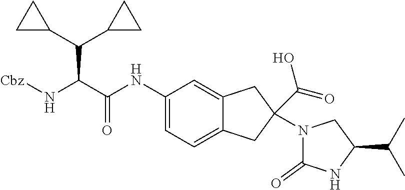 [SMILES: CC(C)[C@@H]1CN(C2(C(=O)O)Cc3ccc(NC(=O)[C@@H](NC(=O)OCc4ccccc4)C(C4CC4)C4CC4)cc3C2)C(=O)N1]

To a solution of methyl 5-((S)-2-(((benzyloxy)carbonyl)amino)-3,3-dicyclopropylpropanamido)-2-((R)-4-isopropyl-2-oxoimidazolidin-1-yl)-2,3-dihydro-1H-indene-2-carboxylate (2.5 g, 4.13 mmol) in THF (47 mL) was added H₂O (10 mL), LiOH·H₂O (654 mg, 8.26 mmol) under N₂. The mixture was stirred at r.t. for 2 hours. The reaction mixture was diluted with HCl acid (30 mL, 0.5M) under stirring and then extracted with EtOAc (35 mL×3). The combined organic phase was washed with brine (40 mL), dried with anhydrous Na₂SO₄, filtered and concentrated in vacuum to obtain the 5-((S)-2-(((benzyloxy)carbonyl)amino)-3,3-dicyclopropylpropanamido)-2-((R)-4-isopropyl-2-oxoimidazolidin-1-yl)-2,3-dihydro-1H-indene-2-carboxylic acid (1.0 g, yield: 50%). ¹H NMR (400 MHz, DMSO-d₆) δ ppm 12.5 (s, 1H), 9.84 (s, 1H), 7.23-7.53 (m, 8H), 7.13-7.11 (m, 1H), 6.75 (d, J=2.4 Hz, 1H), 5.06 (s, 2H), 4.29-4.44 (m, 1H), 3.42-3.52 (m, 2H), 3.36 (s, 2H), 3.16-3.24 (m, 1H), 3.04-3.13 (m, 1H), 1.50-1.47 (m, 1H), 0.70-0.92 (m, 9H), 0.08-0.61 (m, 10H). MS found: [M+H]⁺ 589.4.

### Step 4: benzyl ((2S)-1,1-dicyclopropyl-3-((2-((R)-4-isopropyl-2-oxoimidazolidin-1-yl)-2-(o-tolylcarbamoyl)-2,3-dihydro-1H-inden-5-yl)amino)-3-oxopropan-2-yl)carbamate

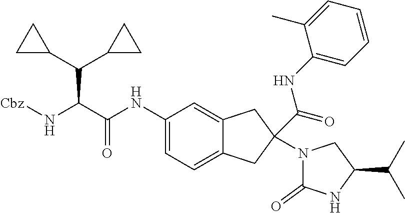 [SMILES: Cc1ccccc1NC(=O)C1(N2C[C@@H](C(C)C)NC2=O)Cc2ccc(NC(=O)[C@@H](NC(=O)OCc3ccccc3)C(C3CC3)C3CC3)cc2C1]

To a solution of 5-((S)-2-(((benzyloxy)carbonyl)amino)-3,3-dicyclopropylpropanamido)-2-((R)-4-isopropyl-2-oxoimidazolidin-1-yl)-2,3-dihydro-1H-indene-2-carboxylic acid (0.15 g, 254.80 umol) in DCM (5 mL) was added o-toluidine (0.027 g, 0.255 mmol), DIEA (65.86 mg, 509.60 umol) and HATU (116.24 mg, 305.76 umol) at 0° C. The mixture was stirred at r.t. for 12 hrs. The reaction mixture was diluted with H₂O (5 mL) and then extracted with DCM (5 mL×3). The combined organic layers were washed with brine, dried over anhydrous Na₂SO₄, filtered and concentrated in vacuum. The crude product was purified by prep-TLC (silica gel, eluent: Petroleum ether/Ethyl acetate (v/v)=5:1, R_{f}=0.1) to obtain benzyl ((2S)-1,1-dicyclopropyl-3-((2-((R)-4-isopropyl-2-oxoimidazolidin-1-yl)-2-(o-tolylcarbamoyl)-2,3-dihydro-1H-inden-5-yl)amino)-3-oxopropan-2-yl)carbamate (0.105 g, yield: 61%).

### Step 5: 5-((S)-2-amino-3,3-dicyclopropylpropanamido)-2-((R)-4-isopropyl-2-oxoimidazolidin-1-yl)-N-(o-tolyl)-2,3-dihydro-1H-indene-2-carboxamide (Int A3)

To a solution of benzyl ((2S)-1,1-dicyclopropyl-3-((2-((R)-4-isopropyl-2-oxoimidazolidin-1-yl)-2-(o-tolylcarbamoyl)-2,3-dihydro-1H-inden-5-yl)amino)-3-oxopropan-2-yl)carbamate (0.105 g, 0.155 mmol) in MeOH (1 mL) and MeNH2·H2O (0.1 mL) was added Pd(OH)2/C (0.1 g, 0.155 mmol) at 20° C. under H₂. The mixture was stirred at 50° C. for 1 hour under H₂ (20 Psi) atmosphere. The reaction mixture was filtered, and the filtrate was concentrated in vacuum. The crude product was purified by prep-TLC (silica gel, eluent: Ethyl acetate/MeOH (v/v)=10/1, R_{f}=0.2) obtain the title compound Int A3 (65 mg, yield: 77%). MS found: [M+H]⁺ 544.5.

Another key intermediates with carboxamide group at 2-position of 2,3-dihydro-1H-indene core fragment were synthesized according to the similar method/procedure as intermediate IntA1 or IntA2 or IntA3, which are known to those skilled in this art; the diverse materials of aldehyde and amino acid were also listed in Table.

| | | Protected Amino |
|---|---|---|
| Int | Structure | acid |
| A4 | 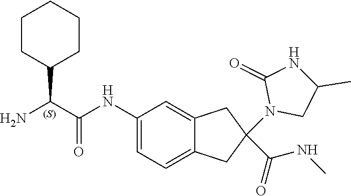 | 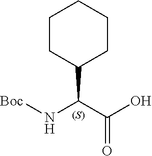 |
| A5 | 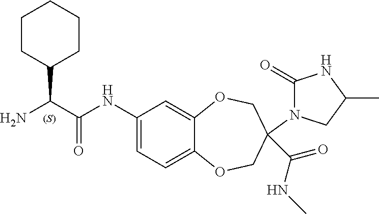 | 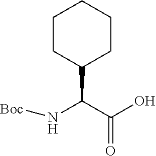 [SMILES: CC(C)(C)OC(=O)N[C@H](C(=O)O)C1CCCCC1] |
| A6 | 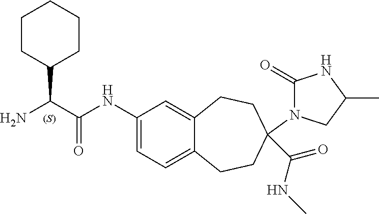 | 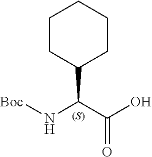 |
| A7 | 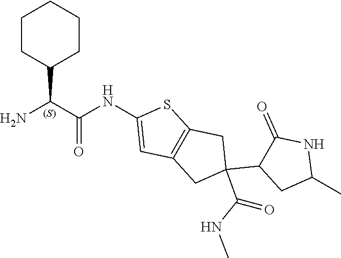 | 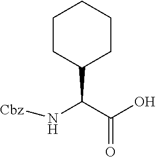 [SMILES: O=C(N[C@H](C(=O)O)C1CCCCC1)OCc1ccccc1] |
| A8 | 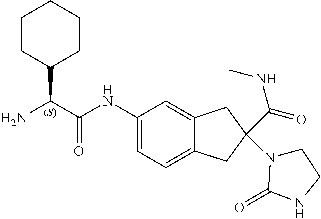 | 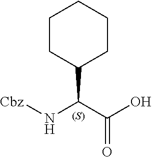 |
| A9 | 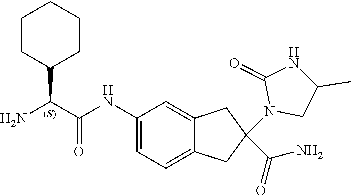 | 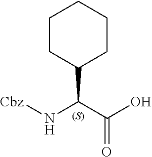 |
| A10 | 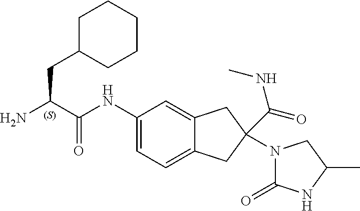 | 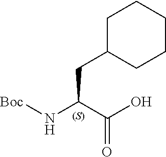 |
| A11 | 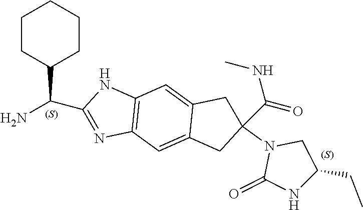 | 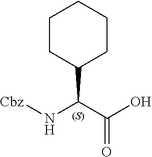 |
| A12 | 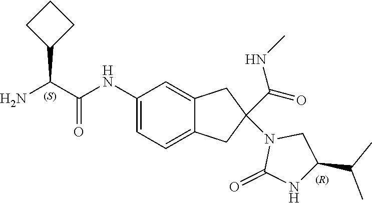 | 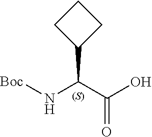 |
| A13 | 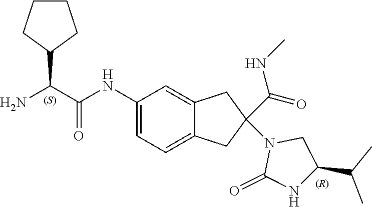 | 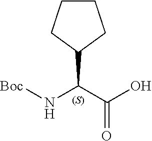 |
| A14 | 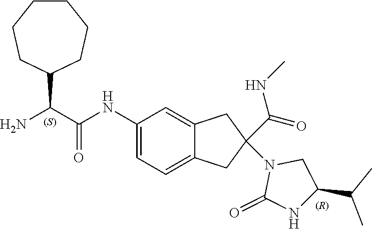 | 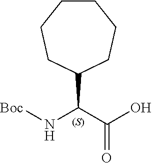 |
| A15 | 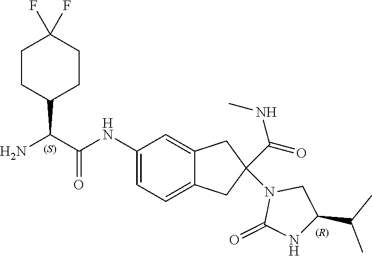 | 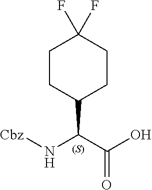 |
| A16 | 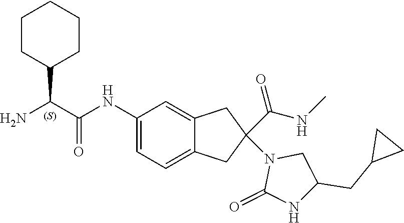 | 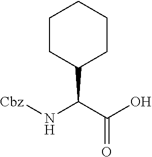 [SMILES: O=C(N[C@H](C(=O)O)C1CCCCC1)OCc1ccccc1] |
| A17 | 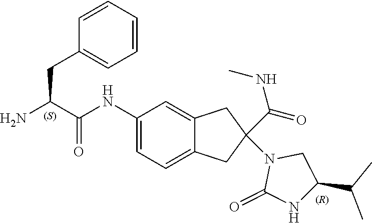 | 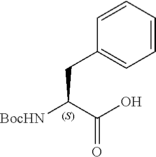 |
| A18 | 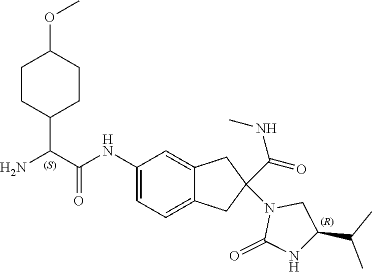 | 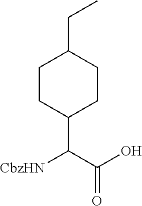 [SMILES: CCC1CCC(C(NC(=O)OCc2ccccc2)C(=O)O)CC1] |
| A19 | 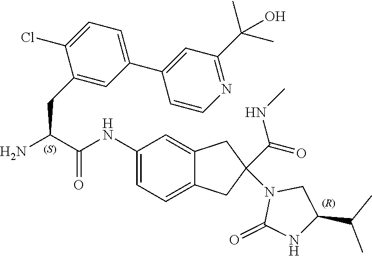 | 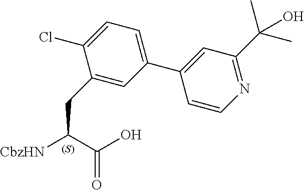 |
| A20 | 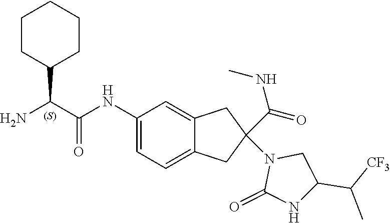 | 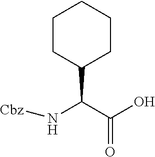 [SMILES: O=C(N[C@H](C(=O)O)C1CCCCC1)OCc1ccccc1] |
| A21 | 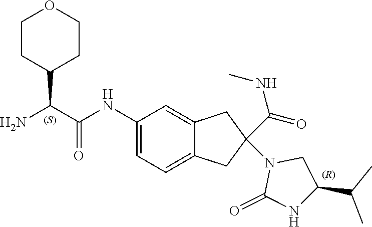 | 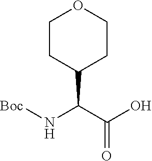 [SMILES: CC(C)(C)OC(=O)N[C@H](C(=O)O)C1CCOCC1] |
| A22 | 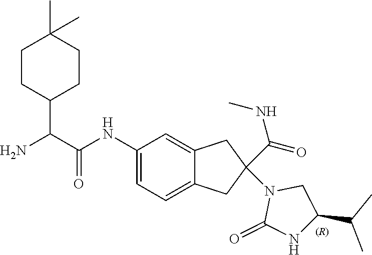 | 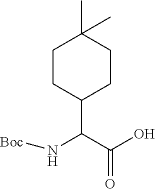 [SMILES: CC1(C)CCC(C(NC(=O)OC(C)(C)C)C(=O)O)CC1] |
| A23 | 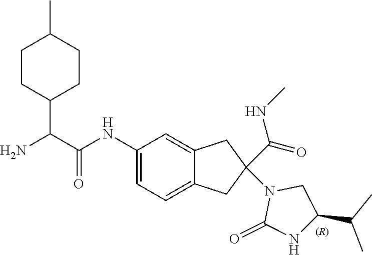 | 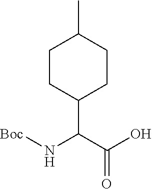 [SMILES: CC1CCC(C(NC(=O)OC(C)(C)C)C(=O)O)CC1] |
| A24 | 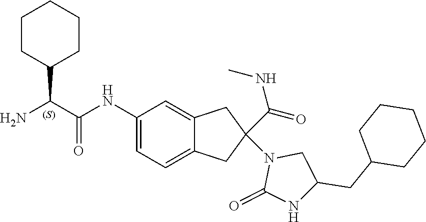 | 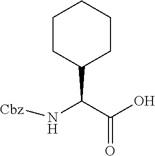 [SMILES: O=C(N[C@H](C(=O)O)C1CCCCC1)OCc1ccccc1] |
| A25 | 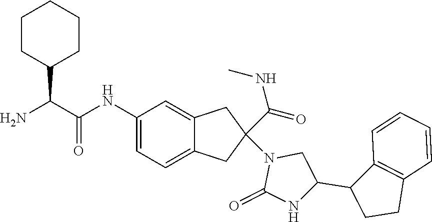 [SMILES: CNC(=O)C1(N2CC(C3CCc4ccccc43)NC2=O)Cc2ccc(NC(=O)[C@@H](N)C3CCCCC3)cc2C1] | 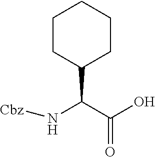 [SMILES: O=C(N[C@H](C(=O)O)C1CCCCC1)OCc1ccccc1] |
| A26 | 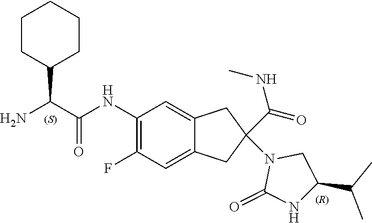 | 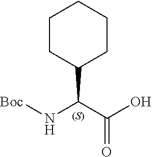 |
| A27 | 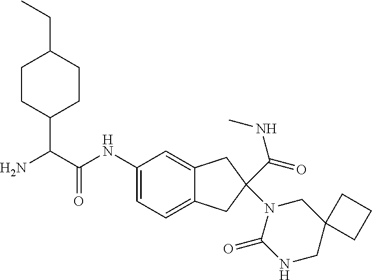 [SMILES: CCC1CCC(C(N)C(=O)Nc2ccc3c(c2)CC(C(=O)NC)(N2CC4(CCC4)CNC2=O)C3)CC1] | 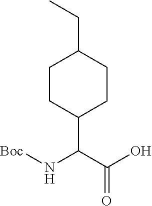 [SMILES: CCC1CCC(C(NC(=O)OC(C)(C)C)C(=O)O)CC1] |
| A28 | 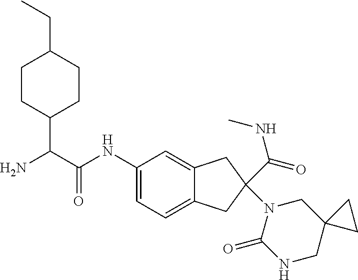 [SMILES: CCC1CCC(C(N)C(=O)Nc2ccc3c(c2)CC(C(=O)NC)(N2CC4(CC4)CNC2=O)C3)CC1] | 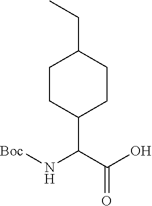 [SMILES: CCC1CCC(C(NC(=O)OC(C)(C)C)C(=O)O)CC1] |
| A29 | 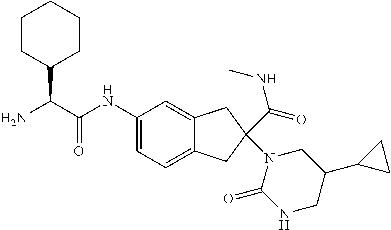 [SMILES: CNC(=O)C1(N2CC(C3CC3)CNC2=O)Cc2ccc(NC(=O)[C@@H](N)C3CCCCC3)cc2C1] | 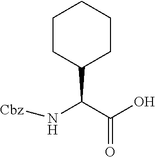 [SMILES: O=C(N[C@H](C(=O)O)C1CCCCC1)OCc1ccccc1] |
| A30 | 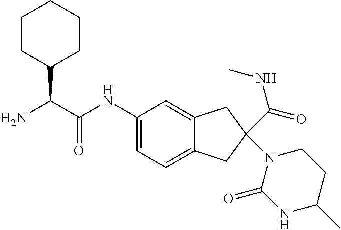 [SMILES: CNC(=O)C1(N2CCC(C)NC2=O)Cc2ccc(NC(=O)[C@@H](N)C3CCCCC3)cc2C1] | 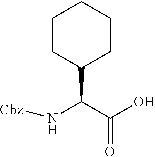 [SMILES: O=C(N[C@H](C(=O)O)C1CCCCC1)OCc1ccccc1] |
| A31 | 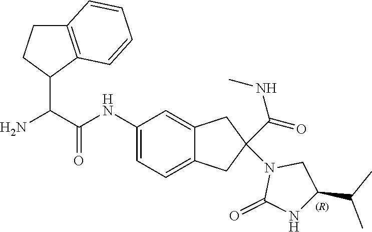 | 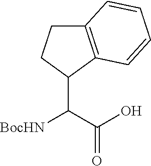 [SMILES: CC(C)(C)OC(=O)NC(C(=O)O)C1CCc2ccccc21] |
| A32 | 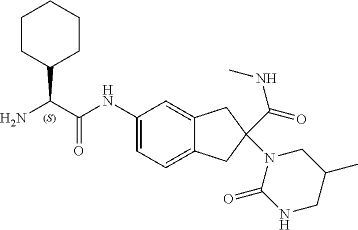 | 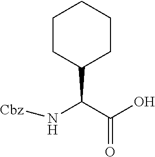 [SMILES: O=C(N[C@H](C(=O)O)C1CCCCC1)OCc1ccccc1] |
| A33 | 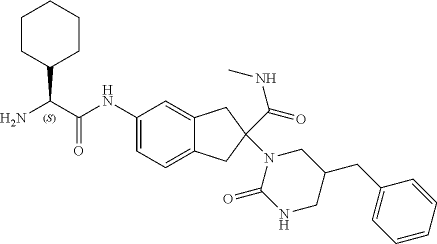 | 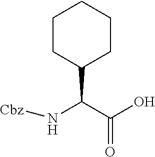 [SMILES: O=C(N[C@H](C(=O)O)C1CCCCC1)OCc1ccccc1] |
| A34 | 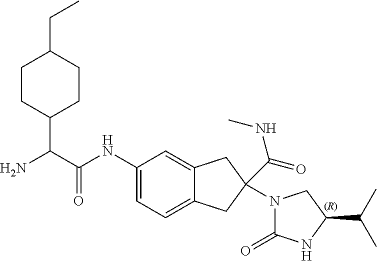 | 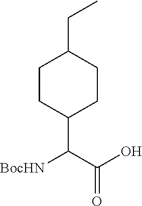 [SMILES: CCC1CCC(C(NC(=O)OC(C)(C)C)C(=O)O)CC1] |
| A35 | 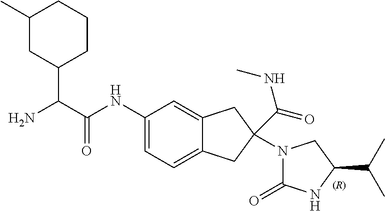 | 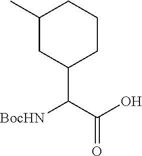 [SMILES: CC1CCCC(C(NC(=O)OC(C)(C)C)C(=O)O)C1] |
| A36 | 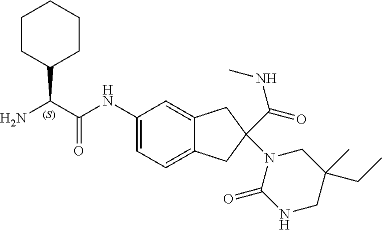 | 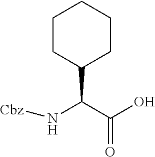 [SMILES: O=C(N[C@H](C(=O)O)C1CCCCC1)OCc1ccccc1] |
| A37 | 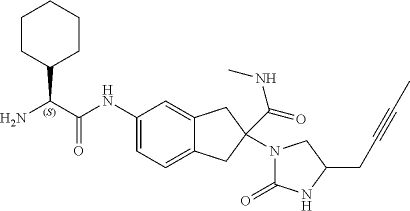 | 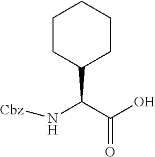 [SMILES: O=C(N[C@H](C(=O)O)C1CCCCC1)OCc1ccccc1] |
| A38 | 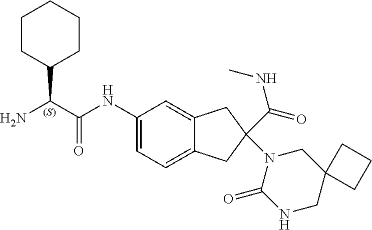 | 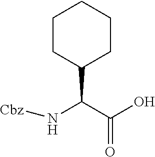 [SMILES: O=C(N[C@H](C(=O)O)C1CCCCC1)OCc1ccccc1] |
| A39 | 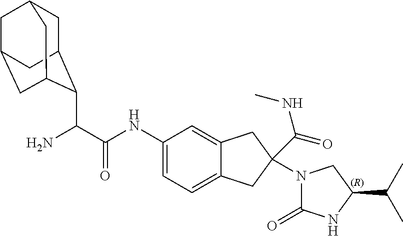 | 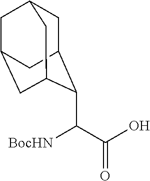 [SMILES: CC(C)(C)OC(=O)NC(C(=O)O)C1C2CC3CC(C2)CC1C3] |
| A40 | 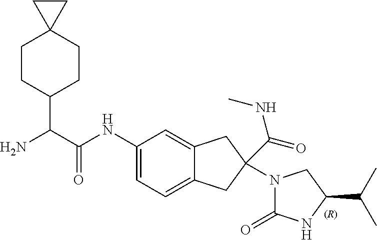 | 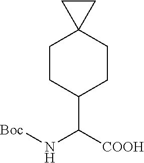 [SMILES: CC(C)(C)OC(=O)NC(C(=O)O)C1CCC2(CC1)CC2] |
| A41 | 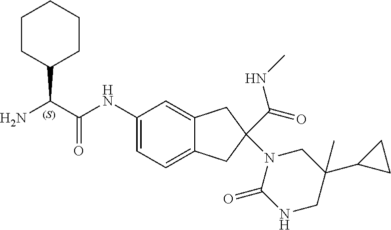 | 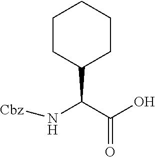 [SMILES: O=C(N[C@H](C(=O)O)C1CCCCC1)OCc1ccccc1] |
| A42 | 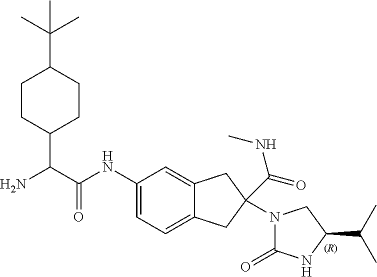 | 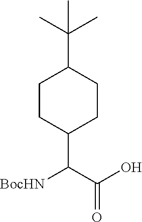 [SMILES: CC(C)(C)OC(=O)NC(C(=O)O)C1CCC(C(C)(C)C)CC1] |
| A43 | 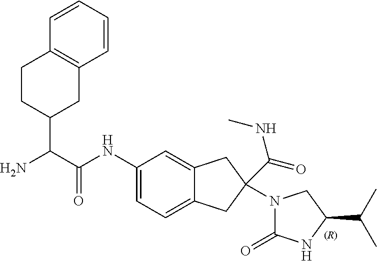 | 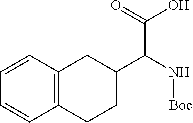 [SMILES: CC(C)(C)OC(=O)NC(C(=O)O)C1CCc2ccccc2C1] |
| A44 | 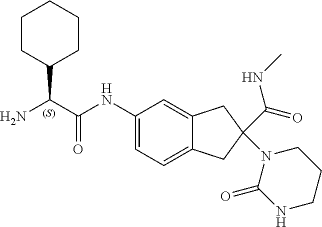 | 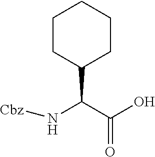 [SMILES: O=C(N[C@H](C(=O)O)C1CCCCC1)OCc1ccccc1] |
| A45 | 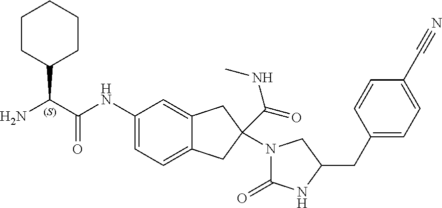 | 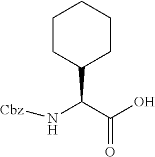 [SMILES: O=C(N[C@H](C(=O)O)C1CCCCC1)OCc1ccccc1] |
| A46 | 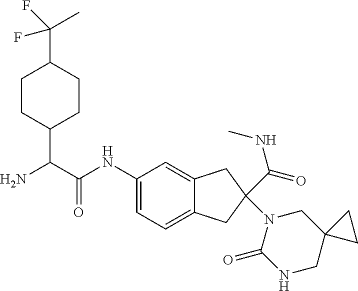 [SMILES: CNC(=O)C1(N2CC3(CC3)CNC2=O)Cc2ccc(NC(=O)C(N)C3CCC(C(C)(F)F)CC3)cc2C1] | 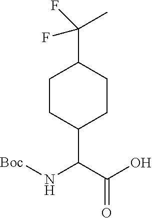 [SMILES: CC(C)(C)OC(=O)NC(C(=O)O)C1CCC(C(C)(F)F)CC1] |
| A47 | 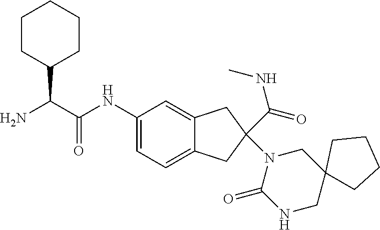 [SMILES: CNC(=O)C1(N2CC3(CCCC3)CNC2=O)Cc2ccc(NC(=O)[C@@H](N)C3CCCCC3)cc2C1] | 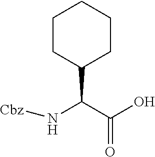 [SMILES: O=C(N[C@H](C(=O)O)C1CCCCC1)OCc1ccccc1] |
| A48 | 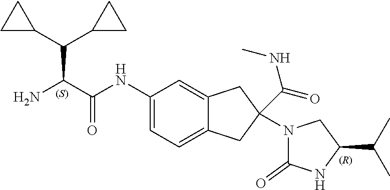 | 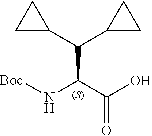 |
| A49 | 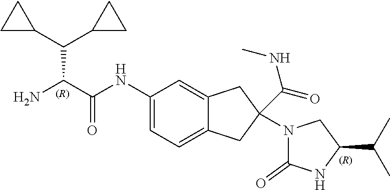 | 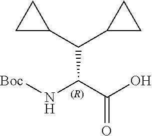 |
| A50 | 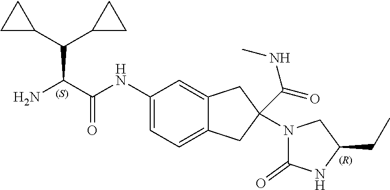 | 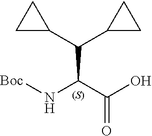 |
| A51 | 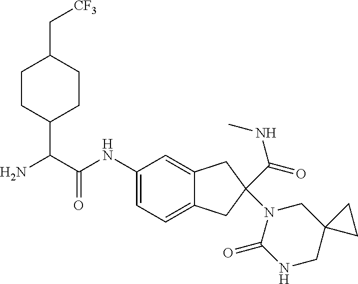 [SMILES: CNC(=O)C1(N2CC3(CC3)CNC2=O)Cc2ccc(NC(=O)C(N)C3CCC(CC(F)(F)F)CC3)cc2C1] | 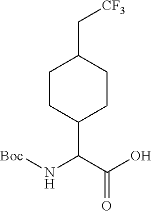 [SMILES: CC(C)(C)OC(=O)NC(C(=O)O)C1CCC(CC(F)(F)F)CC1] |
| A52 | 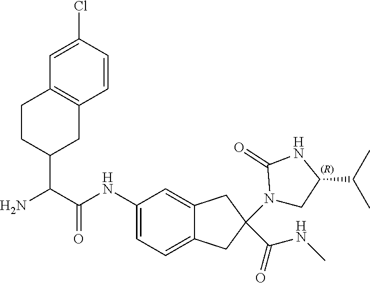 | 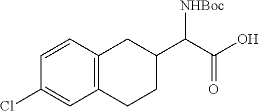 [SMILES: CC(C)(C)OC(=O)NC(C(=O)O)C1CCc2cc(Cl)ccc2C1] |
| A53 | 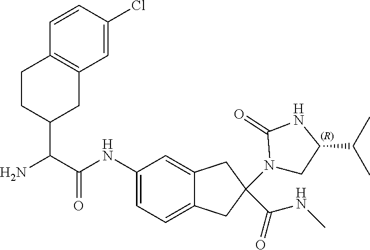 | 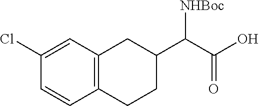 [SMILES: CC(C)(C)OC(=O)NC(C(=O)O)C1CCc2ccc(Cl)cc2C1] |
| A54 | 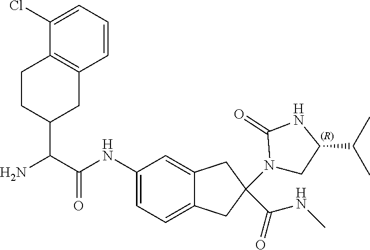 | 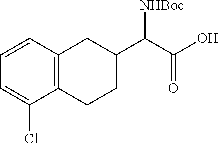 [SMILES: CC(C)(C)OC(=O)NC(C(=O)O)C1CCc2c(Cl)cccc2C1] |
| A55 | 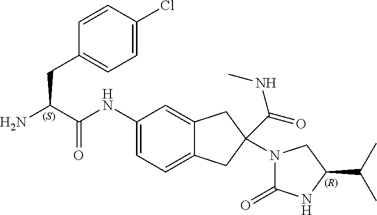 | 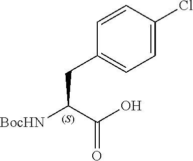 |
| A56 | 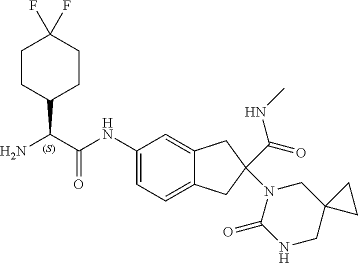 | 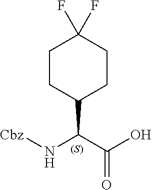 |
| A57 | 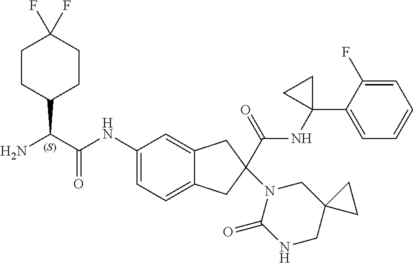 | 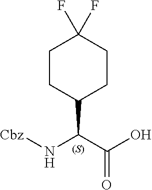 |
| A58 | 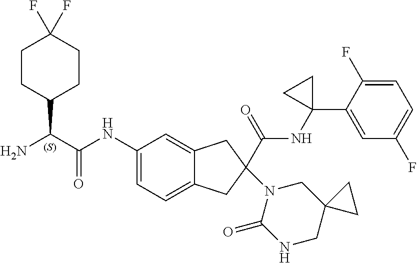 | 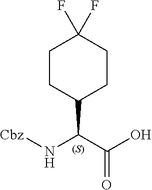 |
| A59 | 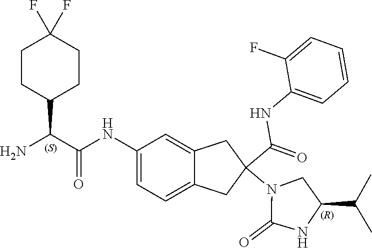 | 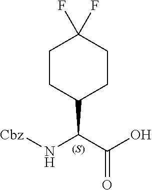 |
| A60 | 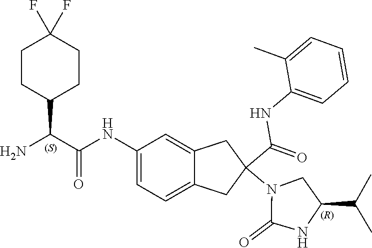 | 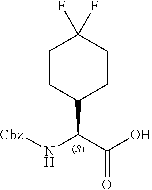 |
| A61 | 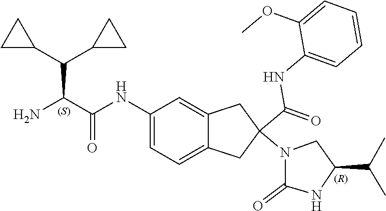 | 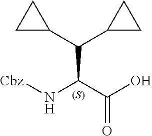 |
| A62 | 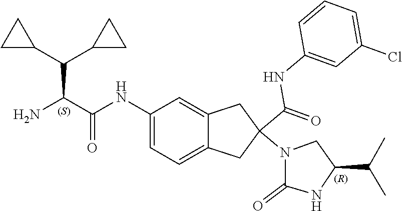 | 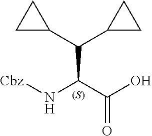 |
| A63 | 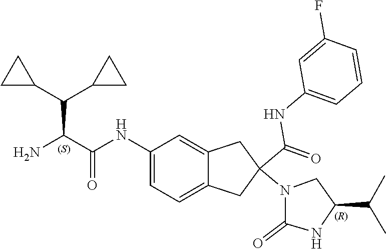 | 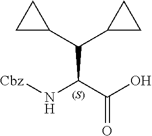 |
| A64 | 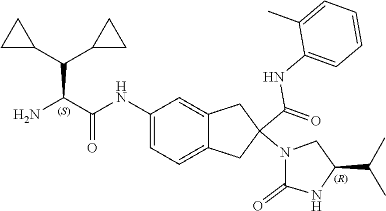 | 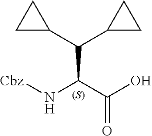 |
| A65 | 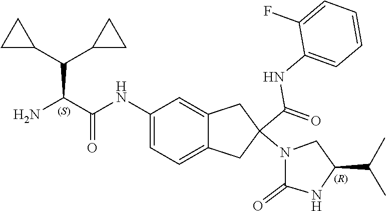 | 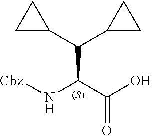 |
| A66 | 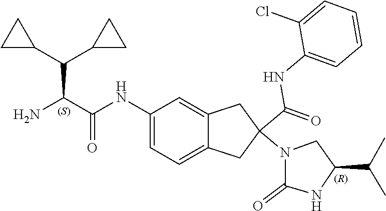 | 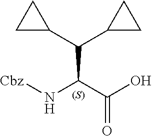 |
| A67 | 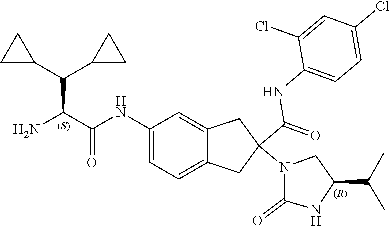 | 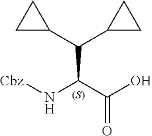 |
| A68 | 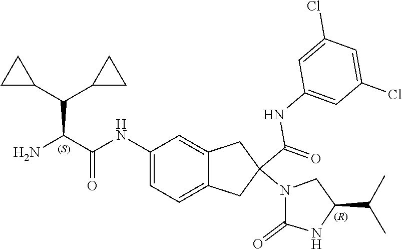 | 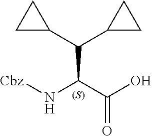 |
| A69 | 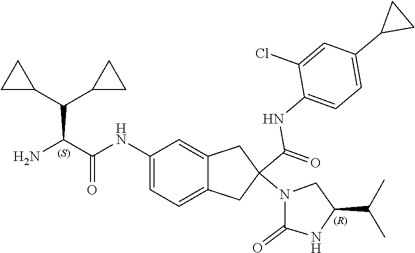 | 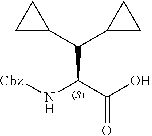 |
| A70 | 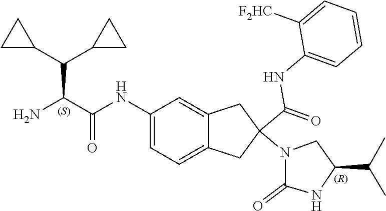 | 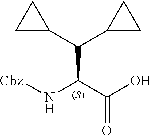 |
| A71 | 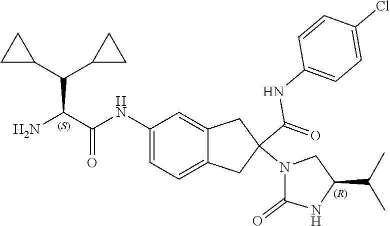 | 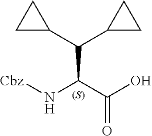 |
| A72 | 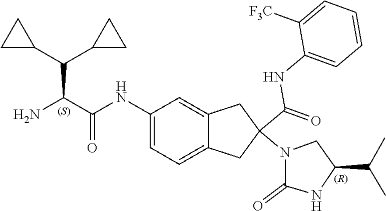 | 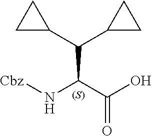 |
| A73 | 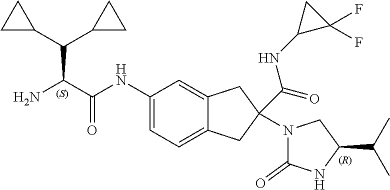 | 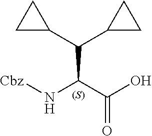 |
| A74 | 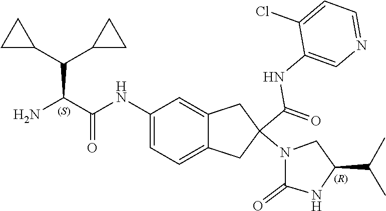 | 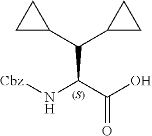 |
| A75 | 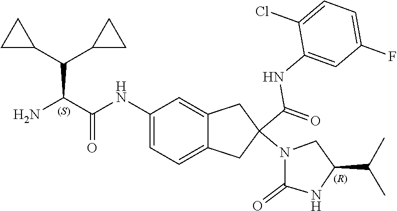 | 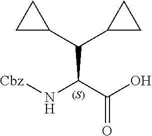 |
| A76 | 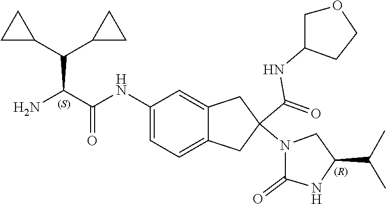 | 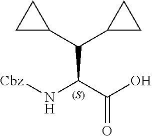 |
| A77 | 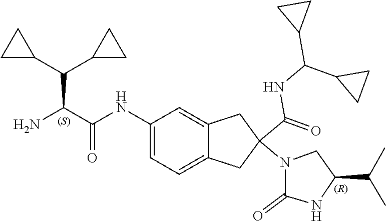 | 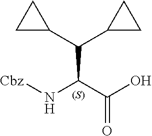 |
| A78 | 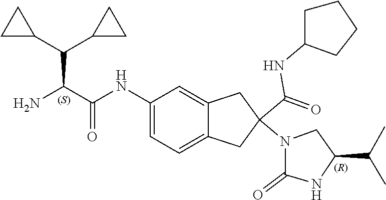 | 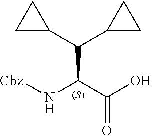 |
| A79 | 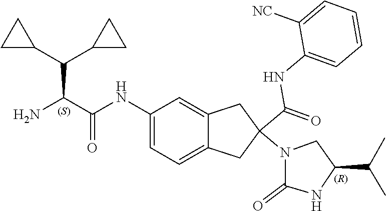 | 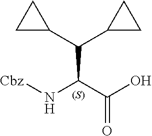 |
| A80 | 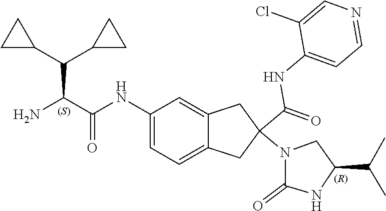 | 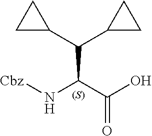 |
| A81 | 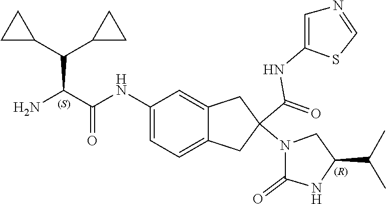 | 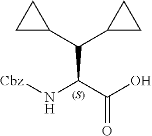 |
| A82 | 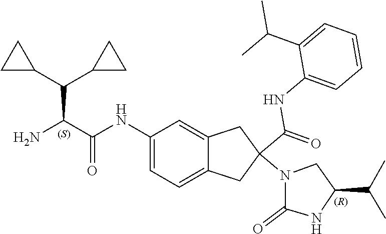 | 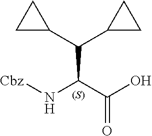 |
| A83 | 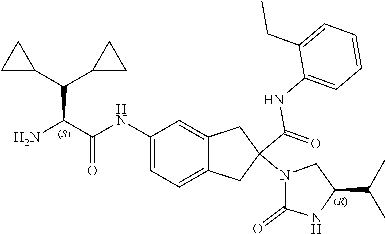 | 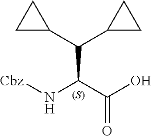 |

| Int | Aldehyde | Data |
|---|---|---|
| A4 | 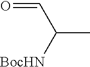 | MS (ESI, m/e) [M + 1]⁺ 428.2 |
| A5 | 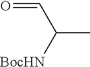 | MS (ESI, m/e) [M + 1]⁺ 460.3 |
| A6 | 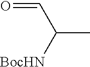 | MS (ESI, m/e) [M + 1]⁺ 456.3 |
| A7 | 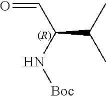 | MS (ESI, m/e) [M + 1]⁺ 434.4. |
| A8 | 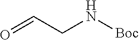 [SMILES: CC(C)(C)OC(=O)NCC=O] | MS (ESI, m/e) [M + 1]⁺ 414.3 |
| A9 | 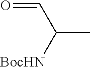 | MS (ESI, m/e) [M + 1]⁺ 414.2 |
| A10 | 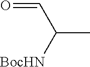 | MS (ESI, m/e) [M + 1]⁺ 442.3 |
| A11 | 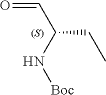 | MS (ESI, m/e) [M + 1]⁺ 439.2 |
| A12 | 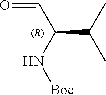 | MS (ESI, m/e) [M + 1]⁺ 428.2 |
| A13 | 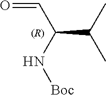 | MS (ESI, m/e) [M + 1]⁺ 442.3 |
| A14 | 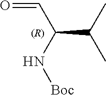 | MS (ESI, m/e) [M + 1]⁺ 470.3 |
| A15 | 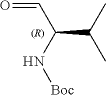 | MS (ESI, m/e) [M + 1]⁺ 492.2. |
| A16 | 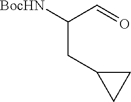 [SMILES: CC(C)(C)OC(=O)NC(C=O)CC1CC1] | MS (ESI, m/e) [M + 1]⁺ 468.2. |
| A17 | 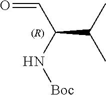 | MS (ESI, m/e) [M + 1]⁺ 464.3. |
| A18 | 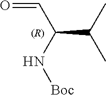 | MS (ESI, m/e) [M + 1]⁺ 486.3. |
| A19 | 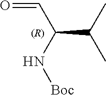 | MS (ESI, m/e) [M + 1]⁺ 633.4. |
| A20 | 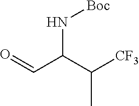 | MS (ESI, m/e) [M + 1]⁺ 510.2. |
| A21 | 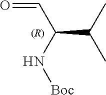 | MS (ESI, m/e) [M + 1]⁺ 458.3. |
| A22 | 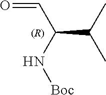 | MS (ESI, m/e) [M + 1]⁺ 484.3 |
| A23 | 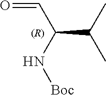 | MS (ESI, m/e) [M + 1]⁺ 470.3 |
| A24 | 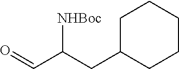 [SMILES: CC(C)(C)OC(=O)NC(C=O)CC1CCCCC1] | MS (ESI, m/e) [M + 1]⁺ 510.4. |
| A25 | 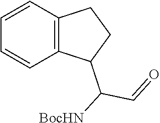 [SMILES: CC(C)(C)OC(=O)NC(C=O)C1CCc2ccccc21] | MS (ESI, m/e) [M + 1]⁺ 530.3. |
| A26 | 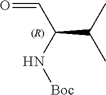 | MS (ESI, m/e) [M + 1]⁺ 474.3 |
| A27 | 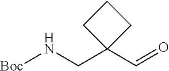 [SMILES: CC(C)(C)OC(=O)NCC1(C=O)CCC1] | MS (ESI, m/e) [M + 1]⁺ 496.3 |
| A28 | 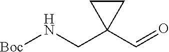 [SMILES: CC(C)(C)OC(=O)NCC1(C=O)CC1] | MS (ESI, m/e) [M + 1]⁺ 482.3 |
| A29 | 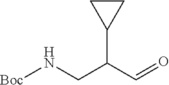 [SMILES: CC(C)(C)OC(=O)NCC(C=O)C1CC1] | MS (ESI, m/e) [M + 1]⁺ 468.4. |
| A30 | 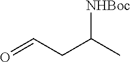 | MS (ESI, m/e) [M + 1]⁺ 442.2. |
| A31 | 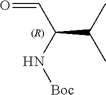 | MS (ESI, m/e) [M + 1]⁺ 490.3. |
| A32 | 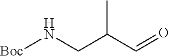 | MS (ESI, m/e) [M + 1]⁺ 442.2. |
| A33 | 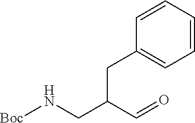 [SMILES: CC(C)(C)OC(=O)NCC(C=O)Cc1ccccc1] | MS (ESI, m/e) [M + 1]⁺ 518.4. |
| A34 | 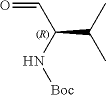 | MS (ESI, m/e) [M + 1]⁺ 484.3. |
| A35 | 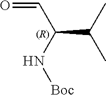 | MS (ESI, m/e) [M + 1]⁺ 470.3. |
| A36 | 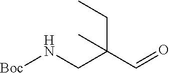 | MS (ESI, m/e) [M + 1]⁺ 470.3. |
| A37 | 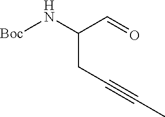 | MS (ESI, m/e) [M + 1]⁺ 466.3. |
| A38 | 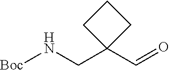 [SMILES: CC(C)(C)OC(=O)NCC1(C=O)CCC1] | MS (ESI, m/e) [M + 1]⁺ 468.3. |
| A39 | 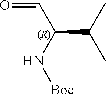 | MS (ESI, m/e) [M + 1]⁺ 508.3. |
| A40 | 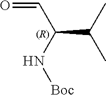 | MS (ESI, m/e) [M + 1]⁺ 482.4. |
| A41 | 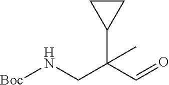 | MS (ESI, m/e) [M + 1]⁺ 482.3 |
| A42 | 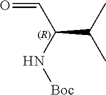 | MS (ESI, m/e) [M + 1]⁺ 512.5. |
| A43 | 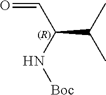 | MS (ESI, m/e) [M + 1]⁺ 504.3. |
| A44 | 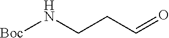 [SMILES: CC(C)(C)OC(=O)NCCC=O] | MS (ESI, m/e) [M + 1]⁺ 428.3. |
| A45 | 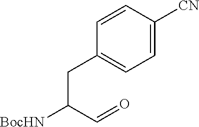 [SMILES: CC(C)(C)OC(=O)NC(C=O)Cc1ccc(C#N)cc1] | MS (ESI, m/e) [M + 1]⁺ 529.3. |
| A46 | 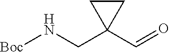 [SMILES: CC(C)(C)OC(=O)NCC1(C=O)CC1] | MS (ESI, m/e) [M + 1]⁺ 519.4 |
| A47 | 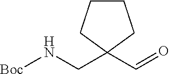 [SMILES: CC(C)(C)OC(=O)NCC1(C=O)CCCC1] | MS (ESI, m/e) [M + 1]⁺ 482.1. |
| A48 | 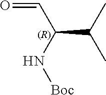 | MS (ESI, m/e) [M + 1]⁺ 468.3 |
| A49 | 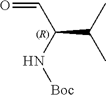 | MS (ESI, m/e) [M + 1]⁺ 468.3 |
| A50 | 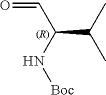 | MS (ESI, m/e) [M + 1]⁺ 454.3. |
| A51 | 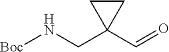 [SMILES: CC(C)(C)OC(=O)NCC1(C=O)CC1] | MS (ESI, m/e) [M + 1]⁺ 536.5. |
| A52 | 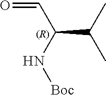 | MS (ESI, m/e) [M + 1]⁺ 538.3. |
| A53 | 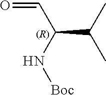 | MS (ESI, m/e) [M + 1]⁺ 538.1. |
| A54 | 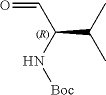 | MS (ESI, m/e) [M + 1]⁺ 538.3. |
| A55 | 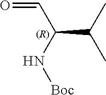 | MS (ESI, m/e) [M + 1]⁺ 498.0. |
| A56 | 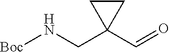 [SMILES: CC(C)(C)OC(=O)NCC1(C=O)CC1] | MS (ESI, m/e) [M + 1]⁺ 490.3 |
| A57 | 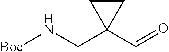 [SMILES: CC(C)(C)OC(=O)NCC1(C=O)CC1] | MS (ESI, m/e) [M + 1]⁺ 610.4. |
| A58 | 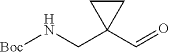 [SMILES: CC(C)(C)OC(=O)NCC1(C=O)CC1] | MS (ESI, m/e) [M + 1]⁺ 628.4. |
| A59 | 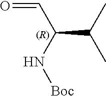 | MS (ESI, m/e) [M + 1]⁺ 572.3. |
| A60 | 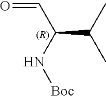 | MS (ESI, m/e) [M + 1]⁺ 568.3. |
| A61 | 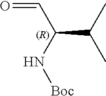 | MS (ESI, m/e) [M + 1]⁺ 560.5. |
| A62 | 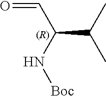 | MS (ESI, m/e) [M + 1]⁺ 564.4. |
| A63 | 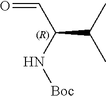 | MS (ESI, m/e) [M + 1]⁺ 632.3. |
| A64 | 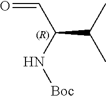 | MS (ESI, m/e) [M + 1]⁺ 544.4. |
| A65 | 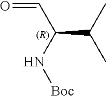 | MS (ESI, m/e) [M + 1]⁺ 548.4. |
| A66 | 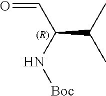 | MS (ESI, m/e) [M + 1]⁺ 564.3. |
| A67 | 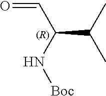 | MS (ESI, m/e) [M + 1]⁺ 598.2. |
| A68 | 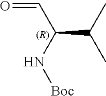 | MS (ESI, m/e) [M + 1]⁺ 598.3. |
| A69 | 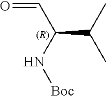 | MS (ESI, m/e) [M + 1]⁺ 604.2. |
| A70 | 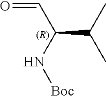 | MS (ESI, m/e) [M + 1]⁺ 580.3. |
| A71 | 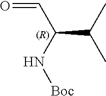 | MS (ESI, m/e) [M + 1]⁺ 564.3. |
| A72 | 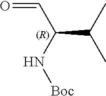 | MS (ESI, m/e) [M + 1]⁺ 598.4. |
| A73 | 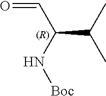 | MS (ESI, m/e) [M + 1]⁺ 530.3. |
| A74 | 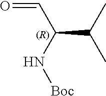 | MS (ESI, m/e) [M + 11]⁺ 565.3. |
| A75 | 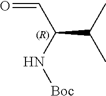 | MS (ESI, m/e) [M + 1]⁺ 582.3. |
| A76 | 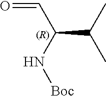 | MS (ESI, m/e) [M + 1]⁺ 524.4. |
| A77 | 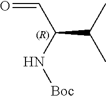 | MS (ESI, m/e) [M + 1]⁺ 548.4. |
| A78 | 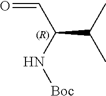 | MS (ESI, m/e) [M + 1]⁺ 522.3. |
| A79 | 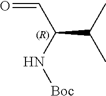 | MS (ESI, m/e) [M + 1]⁺ 555.3. |
| A80 | 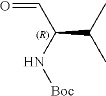 | MS (ESI, m/e) [M + 1]⁺ 565.4. |
| A81 | 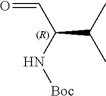 | MS (ESI, m/e) [M + 1]⁺ 537.3. |
| A82 | 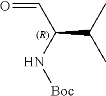 | MS (ESI, m/e) [M + 1]⁺ 572.4. |
| A83 | 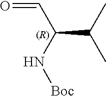 | MS (ESI, m/e) [M + 1]⁺ 558.4. |

The protected amino acid or aldehyde are either commercially available, known in the literature or can be synthesized as outlined in indicated preparation.

### Int B1: 5-(5-amino-2-(1H-benzo[d]imidazol-2-yl)-2,3-dihydro-1H-inden-2-yl)-5,7-diazaspiro[2.5]octan-6-one

### Step 1: 5-nitro-2-(6-oxo-5,7-diazaspiro[2.5]octan-5-yl)-2,3-dihydro-1H-indene-2-carboxylic acid

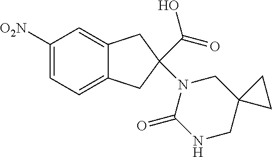 [SMILES: O=C1NCC2(CC2)CN1C1(C(=O)O)Cc2ccc([N+](=O)[O-])cc2C1]

To a solution of N-methyl-5-nitro-2-(6-oxo-5,7-diazaspiro[2.5]octan-5-yl)-2,3-dihydro-1H-indene-2-carboxamide (2.1 g, 6.1 mol, prepared by the procedure similar with 2-((R)-4-isopropyl-2-oxoimidazolidin-1-yl)-N-methyl-5-nitro-2,3-dihydro-1H-indene-2-carboxamide as described in IntA2) in DCM (15 mL) was added TFA (5 mL) and stirred at room temperature for 4 hrs. The reaction mixture was cooled to r.t. and concentrated to remove TFA and solvent in vacuum. The residue was diluted with DCM (50 mL), washed with saturated NaHCO₃ (20 mL), then saturated NH4Cl (20 mL). The organic solution was dried over anhydrous Na₂SO₄, filtered and concentrated in vacuum to obtain 5-nitro-2-(6-oxo-5,7-diazaspiro[2.5]octan-5-yl)-2,3-dihydro-1H-indene-2-carboxylic acid (2 g, yield: 98%). MS found: [M+H]⁺ 332.4.

### Step 2: N-(2-aminophenyl)-5-nitro-2-(6-oxo-5,7-diazaspiro[2.5]octan-5-yl)-2,3-dihydro-1H-indene-2-carboxamide

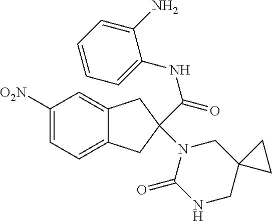 [SMILES: Nc1ccccc1NC(=O)C1(N2CC3(CC3)CNC2=O)Cc2ccc([N+](=O)[O-])cc2C1]

To a solution of 5-nitro-2-(6-oxo-5,7-diazaspiro[2.5]octan-5-yl)-2,3-dihydro-1H-indene-2-carboxylic acid (0.5 g, 1.51 mmol), benzene-1,2-diamine (0.26 g, 1.51 mmol) and TEA (0.3 g, 3 mmol) in DCM (10 mL) was added HATU (0.0.65 g, 1.7 mmol) at 0° C. The mixture was stirred at room temperature for 4 hrs. The reaction mixture was diluted with water (15 mL), extracted with EtOAc (20 mL×3). The combined organic layers were washed with brine, dried over anhydrous Na₂SO₄, filtered and concentrated in vacuum. The residue was purified by column chromatography on silica gel (eluent: Petroleum ether/Ethyl acetate (v/v)=100/1 to 1/1 to obtain N-(2-aminophenyl)-5-nitro-2-(6-oxo-5,7-diazaspiro[2.5]octan-5-yl)-2,3-dihydro-1H-indene-2-carboxamide (0.18 g, yield: 28%). MS found: [M+H]⁺ 422.4.

### Step 3: 5-(2-(1H-benzo[d]imidazol-2-yl)-5-nitro-2,3-dihydro-1H-inden-2-yl)-5,7-diazaspiro[2.5]octan-6-one

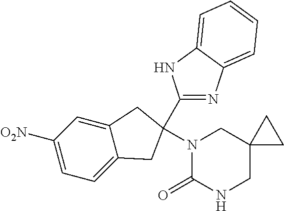 [SMILES: O=C1NCC2(CC2)CN1C1(c2nc3ccccc3[nH]2)Cc2ccc([N+](=O)[O-])cc2C1]

A solution of N-(2-aminophenyl)-5-nitro-2-(6-oxo-5,7-diazaspiro[2.5]octan-5-yl)-2,3-dihydro-1H-indene-2-carboxamide (0.18 g, 0.45 mmol) in AcOH (8 mL) was heated to 50° C. and stirred for 4 hrs. The reaction mixture was diluted with water (15 mL), extracted with EtOAc (20 mL×3). The combined organic layers were washed with brine, dried over anhydrous Na₂SO₄, filtered and concentrated in vacuum. The crude product was purified by column chromatography on silica gel (eluent: Petroleum ether/Ethyl acetate (v/v)=50/1 to 5/1) to obtain 5-(2-(1H-benzo[d]imidazol-2-yl)-5-nitro-2,3-dihydro-1H-inden-2-yl)-5,7-diazaspiro[2.5]octan-6-one (80 mg, yield: 44%). MS found: [M+H]⁺ 404.2.

### Step 4: 5-(5-amino-2-(1H-benzo[d]imidazol-2-yl)-2,3-dihydro-1H-inden-2-yl)-5,7-diazaspiro[2.5]octan-6-one (Int B1)

To a solution of 5-(2-(1H-benzo[d]imidazol-2-yl)-5-nitro-2,3-dihydro-1H-inden-2-yl)-5,7-diazaspiro[2.5]octan-6-one (80 mg, 0.09 mmol) in MeOH (10 mL) was added Raney-Ni (11 mg) and NH3·H₂O (1 mL) under N₂ The suspension was degassed under vacuum and purge with H₂ three times. The mixture was stirred at 50° C. under H₂ atmosphere for 1 hour. The reaction mixture was filtered and concentrated in vacuum to obtain 5-(5-amino-2-(1H-benzo[d]imidazol-2-yl)-2,3-dihydro-1H-inden-2-yl)-5,7-diazaspiro[2.5]octan-6-one (70 mg, yield: 54%). ¹H NMR (400 MHz, DMSO-d6) δ ppm 11.89 (s, 1 H), 7.55-7.38 (m, 2H), 7.07 (s, 2H), 6.79 (d, J=6.8 Hz, 1H), 6.47-6.11 (m, 3H), 4.79 (s, 2H), 4.06-3.82 (m, 2H), 3.27-3.09 (m, 4H), 2.86 (d, J=7.2 Hz, 2H), 0.49-0.27, 4H). [M+H]⁺ 374.2.

### Int B2: 5-(5-amino-2-(imidazo[1,2-a]pyridin-2-yl)-2,3-dihydro-1H-inden-2-yl)-5,7-diazaspiro[2.5]octan-6-one

### Step 1: Ethyl 5-bromo-2-((tert-butoxycarbonyl)amino)-2,3-dihydro-1H-indene-2-carboxylate

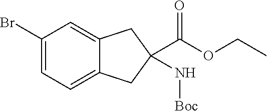 [SMILES: CCOC(=O)C1(NC(=O)OC(C)(C)C)Cc2ccc(Br)cc2C1]

To a solution of ethyl 2-amino-5-bromo-2,3-dihydro-1H-indene-2-carboxylate (4 g, 14.1 mmol) in DCM (60 mL) was added Boc₂O (7.7 g, 35.3 mmol). The mixture was heated to reflux and stirred for 15 hrs. The reaction mixture was cooled to room temperature and concentrated. The residue was purified by column chromatography on silica gel (eluent: EtOAc/PE (v/v)=1/5) to obtain ethyl 5-bromo-2-((tert-butoxycarbonyl)amino)-2,3-dihydro-1H-indene-2-carboxylate (4.8 g, yield: 91%). MS (ESI) m/e [M−56+1]⁺328.1.

### Step 2: 5-bromo-2-((tert-butoxycarbonyl)amino)-2,3-dihydro-1H-indene-2-carboxylic acid

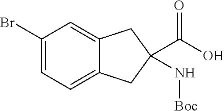 [SMILES: CC(C)(C)OC(=O)NC1(C(=O)O)Cc2ccc(Br)cc2C1]

To a solution of Ethyl 5-bromo-2-((tert-butoxycarbonyl)amino)-2,3-dihydro-1H-indene-2-carboxylate (3.45 g, 9.0 mmol) in THF (45 mL) was added NaOH solution (1.44 g in 15 mL H₂O) dropwise under stirring at room temperature. The resulting mixture was heated to 55° C. and stirred for 16 hrs. The reaction mixture was cooled to room temperature and concentrated in vacuum. The residue was diluted with water and the mixture was adjusted to pH˜5 with HCl acid (1 N), and was then extracted with EtOAc (50×3 mL). The organic layer was washed with brine, dried and concentrated in vacuum. The crude product was stirred in EtOAc/PE (1:1) for 10 min at room temperature. The solid was collected by filtration and dried in vacuum to obtain 5-bromo-2-((tert-butoxycarbonyl)amino)-2,3-dihydro-1H-indene-2-carboxylic acid (3 g, yield: 93%). MS (ESI) m/e [M+23]⁺378.1.

### Step 3: Tert-butyl (5-bromo-2-(methoxy(methyl)carbamoyl)-2,3-dihydro-1H-inden-2-yl)carbamate

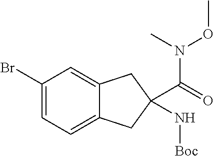 [SMILES: CON(C)C(=O)C1(NC(=O)OC(C)(C)C)Cc2ccc(Br)cc2C1]

To a solution of 5-bromo-2-((tert-butoxycarbonyl)amino)-2,3-dihydro-1H-indene-2-carboxylic acid (2.3 g, 6.5 mmol) in DCM (50 mL) was added N,O-dimethylhydroxylamine hydrochloride (698 mg, 7.1 mmol) and HATU (2.7 g, 7.1 mmol), DIPEA (3.3 g, 25.9 mmol) dropwise at room temperature. The resulting mixture was stirred at room temperature for 2 hrs. The reaction mixture was diluted with EtOAc. The organic layer was washed with saturated NaHCO₃ solution (aq.) and brine, dried and concentrated in vacuum. The residue was purified by column chromatography on silica gel (eluent: EtOAc/PE (v/v)=1/2) to obtain tert-butyl (5-bromo-2-(methoxy(methyl)carbamoyl)-2,3-dihydro-1H-inden-2-yl)carbamate (2.3 g, yield: 88%). MS (ESI) m/e [M−56+1]+343.2.

### Step 4: Tert-butyl (2-acetyl-5-bromo-2,3-dihydro-1H-inden-2-yl)carbamate

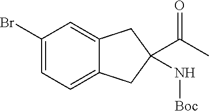 [SMILES: CC(=O)C1(NC(=O)OC(C)(C)C)Cc2ccc(Br)cc2C1]

To a solution of tert-butyl (5-bromo-2-(methoxy(methyl)carbamoyl)-2,3-dihydro-1H-inden-2-yl)carbamate (1.55 g, 3.9 mmol) in THF (25 mL) was added CH₃MgCl (4 mL, 11.7 mmol) at below −10° C. under N₂ atmosphere. The resulting mixture was stirred for 1 hour at 0° C. The reaction mixture was poured into saturated aq. NH₄Cl (30 mL) at 0° C. and then extracted with EtOAc (30 mL×3). The combined organic layer was washed with brine, dried and concentrated in vacuum. The residue was purified by column chromatography on silica gel (eluent: EtOAc/PE (v/v)=1/6) to obtain tert-butyl (2-acetyl-5-bromo-2,3-dihydro-1H-inden-2-yl)carbamate (1.3 g, yield: 94%). MS (ESI) m/e [M+23]⁺376.1.

### Step 5: Tert-butyl (5-bromo-2-(1-((trimethylsilyl)oxy)vinyl)-2,3-dihydro-1H-inden-2-yl)carbamate

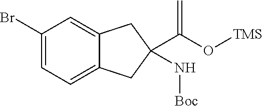 [SMILES: C=C(O[Si](C)(C)C)C1(NC(=O)OC(C)(C)C)Cc2ccc(Br)cc2C1]

Tert-butyl (2-acetyl-5-bromo-2,3-dihydro-1H-inden-2-yl)carbamate (1.1 g, 3.1 mmol) was dissolved into THF (15 mL) and cooled to −70° C. under N₂ atmosphere. To this solution was added LDA (3.1 mL, 6.2 mmol) dropwise with stirring at −70° C. under N₂ atmosphere. After the resulting mixture was stirred for 30 min, TMSCl (680 mg, 6.2 mmol) was added at −70° C. After further stirred at −60° C. for 1 hour, the reaction mixture was quenched by NH₄Cl (aq.) below −20° C. and then was diluted with EtOAc. The organic layer was separated, washed with water and brine, dried and concentrated in vacuum to obtain crude product (1.5 g), which used in next step directly.

### Step 6: Tert-butyl (5-bromo-2-(2-bromoacetyl)-2,3-dihydro-1H-inden-2-yl)carbamate

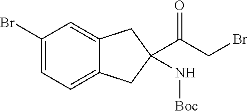 [SMILES: CC(C)(C)OC(=O)NC1(C(=O)CBr)Cc2ccc(Br)cc2C1]

To a solution of tert-butyl (5-bromo-2-(1-((trimethylsilyl)oxy)vinyl)-2,3-dihydro-1H-inden-2-yl)carbamate (1.5 g, crude) in THF (20 mL) was added NBS (625 mg, 3.5 mmol) in several portions at 0° C. The mixture was stirred for 1 hour at 0° C. The reaction mixture was quenched by NaHCO₃ (aq.) and diluted with EtOAc (30 mL). The organic layer was separated, washed with water and brine, dried and concentrated. The residue was purified by column chromatography on silica gel (eluent: EtOAc/PE (v/v)=1/7) to obtain tert-butyl (5-bromo-2-(2-bromoacetyl)-2,3-dihydro-1H-inden-2-yl)carbamate (740 mg, yield: 55% for two steps). MS (ESI) m/e [M+23]⁺ 456.1.

### Step 7: Tert-butyl (5-bromo-2-(imidazo[1,2-a]pyridin-2-yl)-2,3-dihydro-1H-inden-2-yl)carbamate

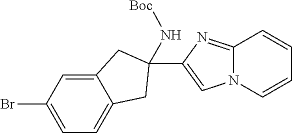 [SMILES: CC(C)(C)OC(=O)NC1(c2cn3ccccc3n2)Cc2ccc(Br)cc2C1]

A solution of Tert-butyl (5-bromo-2-(2-bromoacetyl)-2,3-dihydro-1H-inden-2-yl)carbamate (740 mg, 1.72 mmol) and pyridin-2-amine (162 mg, 1.72 mmol) in MeOH (10 mL) was heated to 80° C. and stirred for 24 h under N₂ atmosphere. The reaction mixture was cooled to room temperature and concentrated in vacuum. The residue was purified by column chromatography on silica gel (eluent: EtOAc/PE (v/v)=1/2) to obtain tert-butyl (5-bromo-2-(imidazo[1,2-a]pyridin-2-yl)-2,3-dihydro-1H-inden-2-yl)carbamate (250 mg, yield: 34%). MS (ESI) m/e [M+1]⁺ 428.2.

### Step 8: 5-bromo-2-(imidazo[1,2-a]pyridin-2-yl)-2,3-dihydro-1H-inden-2-amine

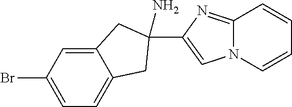 [SMILES: NC1(c2cn3ccccc3n2)Cc2ccc(Br)cc2C1]

To a solution of tert-butyl (5-bromo-2-(imidazo[1,2-a]pyridin-2-yl)-2,3-dihydro-1H-inden-2-yl)carbamate (250 mg, 0.59 mmol) in DCM (16 mL) was added HCl (4N in dioxane, 4 mL) dropwise at room temperature. The resulting mixture was stirred for 2 hrs at room temperature. The reaction mixture was then concentrated in vacuum. 5-bromo-2-(imidazo[1,2-a]pyridin-2-yl)-2,3-dihydro-1H-inden-2-amine (180 mg, crude) as HCl salts. MS (ESI) m/e [M+1]⁺ 328.1.

### Step 9: Tert-butyl ((1-(((5-bromo-2-(imidazo[1,2-a]pyridin-2-yl)-2,3-dihydro-1H-inden-2-yl)amino)methyl)cyclopropyl)-methyl)carbamate

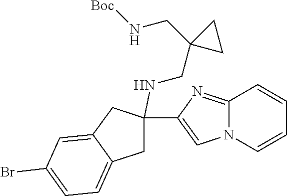 [SMILES: CC(C)(C)OC(=O)NCC1(CNC2(c3cn4ccccc4n3)Cc3ccc(Br)cc3C2)CC1]

A mixture of 5-bromo-2-(imidazo[1,2-a]pyridin-2-yl)-2,3-dihydro-1H-inden-2-amine (180 mg, 0.55 mmol) and tert-butyl ((1-formylcyclopropyl)methyl)carbamate (154 mg, 0.77 mmol) in DCM (10 mL) was stirred at room temperature for 1 hour. To this mixture was then added NaBH₃CN (351 mg, 1.66 mmol) in portions at 0° C. The resulting mixture was stirred at room temperature overnight. The reaction mixture was quenched by NaHCO₃ (aq.) and extracted with EtOAc (30 mL). The organic layer was washed with brine, dried and concentrated in vacuum. The residue was purified by Prep-TLC (eluent: DCM/EtOAc (v/v)=1/1) to obtain tert-butyl ((1-(((5-bromo-2-(imidazo[1,2-a]pyridin-2-yl)-2,3-dihydro-1H-inden-2-yl)amino)methyl)cyclopropyl)-methyl)carbamate (200 mg, yield: 67% for two steps). MS (ESI) m/e [M+1]⁺ 511.3.

### Step 10: N-((1-(aminomethyl)cyclopropyl)methyl)-5-bromo-2-(imidazo[1,2-a]pyridin-2-yl)-2,3-dihydro-1H-inden-2-amine

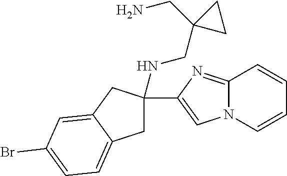 [SMILES: NCC1(CNC2(c3cn4ccccc4n3)Cc3ccc(Br)cc3C2)CC1]

To a solution of tert-butyl ((1-(((5-bromo-2-(imidazo[1,2-a]pyridin-2-yl)-2,3-dihydro-1H-inden-2-yl)amino)methyl)cyclopropyl)-methyl)carbamate (200 mg, 0.39 mmol) in DCM (9 mL) was added HCl (4N in dioxane, 3 mL) dropwise at 0° C. The resulting mixture was stirred for 2 h at room temperature. The reaction mixture was then concentrated and N-((1-(aminomethyl)cyclopropyl)methyl)-5-bromo-2-(imidazo[1,2-a]pyridin-2-yl)-2,3-dihydro-1H-inden-2-amine HCl salts (200 mg, crude) was obtained, which used in next step directly. MS (ESI) m/e [M+1]⁺ 411.2.

### Step 11: 5-(5-bromo-2-(imidazo[1,2-a]pyridin-2-yl)-2,3-dihydro-1H-inden-2-yl)-5,7-diazaspiro[2.5]octan-6-one

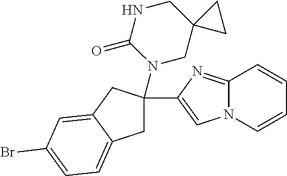 [SMILES: O=C1NCC2(CC2)CN1C1(c2cn3ccccc3n2)Cc2ccc(Br)cc2C1]

To a solution of N-((1-(aminomethyl)cyclopropyl)methyl)-5-bromo-2-(imidazo[1,2-a]pyridin-2-yl)-2,3-dihydro-1H-inden-2-amine (200 mg) and TEA (1 mL) in THF (10 mL) was added CDI (198 mg, 1.22 mmol). The mixture was heated to 60° C. and stirred for 1 hour. The reaction mixture was cooled to room temperature, diluted with EtOAc, washed with water and brine, dried and concentrated in vacuum. The crude product was purified by Prep-TLC (eluent: DCM/MeOH (v/v)=15/1) to obtain 5-(5-bromo-2-(imidazo[1,2-a]pyridin-2-yl)-2,3-dihydro-1H-inden-2-yl)-5,7-diazaspiro[2.5]octan-6-one (100 mg, yield: 58% for two steps). MS (ESI) m/e [M+1]⁺ 437.2.

### Step 12: 5-(5-((diphenylmethylene)amino)-2-(imidazo[1,2-a]pyridin-2-yl)-2,3-dihydro-1H-inden-2-yl)-5,7-diazaspiro[2.5]octan-6-one

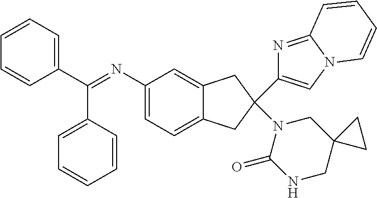 [SMILES: O=C1NCC2(CC2)CN1C1(c2cn3ccccc3n2)Cc2ccc(N=C(c3ccccc3)c3ccccc3)cc2C1]

To a solution of 5-(5-bromo-2-(imidazo[1,2-a]pyridin-2-yl)-2,3-dihydro-1H-inden-2-yl)-5,7-diazaspiro[2.5]octan-6-one (100 mg, 0.23 mmol) in dioxane (15 mL) were added diphenylmethanimine (62.3 mg, 0.34 mmol), Pd₂dba₃ (21 mg, 0.02 mmol), Xantphos (27 mg, 0.04 mmol) and Cs₂CO₃ (150 mg, 0.46 mmol). The mixture was degassed with N₂, heated to 100° C. and then stirred for 6 hrs under N₂ atmosphere. The reaction mixture was cooled to room temperature and diluted with DCM/MeOH (v/v=15/1). After solids were filtered out, the filtrate was concentrated and purified by Prep-TLC (eluent: DCM/MeOH (v/v)=20/1) to obtain 5-(5-((diphenylmethylene)amino)-2-(imidazo[1,2-a]pyridin-2-yl)-2,3-dihydro-1H-inden-2-yl)-5,7-diazaspiro[2.5]octan-6-one (58 mg, yield: 47%). MS (ESI) m/e [M+1]+538.4.

### Step 13: 5-(5-amino-2-(imidazo[1,2-a]pyridin-2-yl)-2,3-dihydro-1H-inden-2-yl)-5,7-diazaspiro[2.5]octan-6-one (Int B2)

To a solution of 5-(5-((diphenylmethylene)amino)-2-(imidazo[1,2-a]pyridin-2-yl)-2,3-dihydro-1H-inden-2-yl)-5,7-diazaspiro[2.5]octan-6-one (58 mg, 0.11 mmol) in THF (5 mL) was added HCl (3N, 1 mL) at room temperature and stirred for 30 min. The resulting reaction mixture was poured into saturated aq. NaHCO₃ and then extracted with EA (30×3 mL). The combined organic layer were washed with brine, dried, filtered and concentrated in vacuum. The residue was purified by Prep-TLC (eluent: DCM/MeOH (v/v)=10/1) to obtain 5-(5-amino-2-(imidazo[1,2-a]pyridin-2-yl)-2,3-dihydro-1H-inden-2-yl)-5,7-diazaspiro[2.5]octan-6-one (22 mg, yield: 55%). MS (ESI) m/e [M+1]⁺ 374.3.

### Int C1: (4S)-1-(1-(6-amino-2,3-dihydrobenzofuran-2-yl)ethyl)-4-(trifluoromethyl)imidazolidin-2-one

### Step 1: 1-(6-nitrobenzofuran-2-yl)ethan-1-one

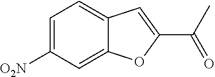

To a solution of 2-hydroxy-4-nitrobenzaldehyde (2 g, 12 mmol) and 1-chloropropan-2-one (1.1 g, 12 mmol) in CH₃CN (20 mL) was added K₂CO₃ (3.3 g, 24 mmol). The mixture was stirred for 4 hrs at room temperature. The reaction mixture was poured into water (40 mL) and then extracted with EtOAc (20 mL×3). The combined organic layers were washed with brine, dried over anhydrous Na₂SO₄, filtered and concentrated in vacuum. The residue was purified by column chromatography on silica gel (eluent: PE/EA (v/v)=100/1 to 10/1) to obtain 1-(6-nitrobenzofuran-2-yl)ethanone (0.61 g, yield: 25%). MS (ESI) m/e [M+1]⁺ 206.2.

### Step 2: (S)-1,1,1-trifluoro-3-((1-(6-nitrobenzofuran-2-yl)ethylidene)amino)propan-2-amine

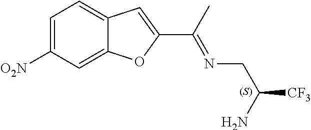

To a solution of 1-(6-nitrobenzofuran-2-yl)ethanone (0.4 g, 1.9 mmol) and (S)-3,3,3-trifluoropropane-1,2-diamine hydrochloride (0.164 g, 2.9 mmol) in DCM (10 mL) was added TEA (0.101 g, 19.5 mmol) and 4A MS (1 g). The mixture was stirred at 45° C. for 3 hrs. After the reaction mixture was filtered, the filtrate was concentrated under reduced pressure. The crude product was purified by column chromatography on silica gel (eluent: PE/EA (v/v)=100/1 to 0/1) to give (S)-3,3,3-trifluoro-N1-(1-(6-nitrobenzofuran-2-yl)ethylidene)propane-1,2-diamine (0.5 g, yield: 81%). MS (ESI) m/e [M+1]+316.1.

### Step 3: (2S)-3,3,3-trifluoro-N1-(1-(6-nitrobenzofuran-2-yl)ethyl)propane-1,2-diamine

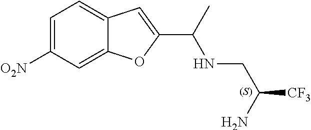

To a solution of (S)-3,3,3-trifluoro-N1-(1-(6-nitrobenzofuran-2-yl)ethylidene)propane-1,2-diamine (0.5 g, 1.6 mmol) in MeOH (10 mL) was added NaBH₃CN (1 g, 16 mmol). The mixture was stirred at 70° C. for 12 hrs. The mixture was poured into water (20 mL) and then extracted with EtOAc (10 mL×3). The combined organic layers were washed with brine, dried over anhydrous Na₂SO₄, filtered and concentrated in vacuum. The residue was purified The crude product was purified by column chromatography on silica gel (eluent: DCM/CH₃OH(v/v)=100/1 to 10/1) to obtain (2S)-3,3,3-trifluoro-N1-(1-(6-nitrobenzofuran-2-yl)ethyl)propane-1,2-diamine (0.32 g, yield: 64%). MS (ESI) m/e [M+1]+318.2.

### Step 4: (4S)-1-(1-(6-nitrobenzofuran-2-yl)ethyl)-4-(trifluoromethyl)imidazolidin-2-one

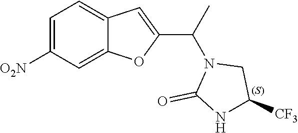

To a solution of (2S)-3,3,3-trifluoro-N1-(1-(6-nitrobenzofuran-2-yl)ethyl)propane-1,2-diamine (1.2 g, 3.8 mmol) in THF (15 mL) was added CDI (0.92 g, 5.7 mmol) and TEA (0.5 mL) at 20° C. The mixture was stirred at 60° C. for 6 hrs. The reaction mixture was diluted with EtOAc (30 mL) and then were washed with brine. The organic layer was dried over anhydrous Na₂SO₄, filtered and concentrated in vacuum. The residue was purified by column chromatography on silica gel (eluent: PE/EAE (v/v)=100/1 to 10/1) to obtain (4S)-1-(1-(6-nitrobenzofuran-2-yl)ethyl)-4-(trifluoromethyl)imidazolidin-2-one (0.34 g, yield: 26%). MS (ESI) m/e [M+1]⁺ 344.3.

### Step 5: (4S)-1-(1-(6-amino-2,3-dihydrobenzofuran-2-yl)ethyl)-4-(trifluoromethyl)imidazolidin-2-one (Int C1)

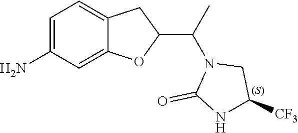

To a solution of (4S)-1-(1-(6-nitrobenzofuran-2-yl)ethyl)-4-(trifluoromethyl)imidazolidin-2-one (0.32 g, 0.932 mmol) in i-PrOH (5 mL) was added Pd/C (0.1 g, 0.932 mmol) at room temperature. The mixture was heated to 50° C. and stirred and hydrogenated for 2 hrs under H₂ (15 Psi) atmosphere. The reaction mixture was filtered, and the filtrate was concentrated in vacuum. (4S)-1-(1-(6-amino-2,3-dihydrobenzofuran-2-yl)ethyl)-4-(trifluoromethyl)imidazolidin-2-one (282 mg) was obtained. ¹H NMR (400 MHz, CDCl₃) δ ppm 7.02-6.84 (m, 1H), 6.79-6.12 (m, 2H), 5.10-4.92 (m, 1H), 4.91-4.72 (m, 1H), 4.20-4.09 (m, 1H), 4.06-3.90 (m, 1H), 3.83-3.66 (m, 1H), 3.65-3.49 (m, 2H), 3.48-2.80 (m, 1H), 1.58-1.12 (m, 3H). MS (ESI, m/e) [M+H]⁺ 316.1.

### Example 32: N-((1S)-cyclohexyl(6-((R)-4-ethyl-2-oxoimidazolidin-1-yl)-6-(methylcarbamoyl)-1,5,6,7-tetrahydroindeno[5,6-d]imidazol-2-yl)methyl)-4-methyl-1,2,5-oxadiazole-3-carboxamide

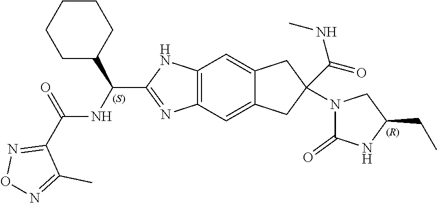

### Step 1: 2-amino-N-methyl-5,6-dinitro-2,3-dihydro-1H-indene-2-carboxamide

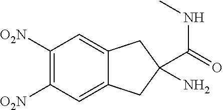 [SMILES: CNC(=O)C1(N)Cc2cc([N+](=O)[O-])c([N+](=O)[O-])cc2C1]

To the mixture of H₂SO₄ (100 mL) and HNO₃ (25 mL) was slowly added 2-amino-N-methyl-2,3-dihydro-1H-indene-2-carboxamide (19 g, 100 mmol, dissolved in 10 mL HOAc) in batches at 0° C. After addition, the mixture was stirred for 4 hrs at room temperature. The reaction mixture was poured into saturated NaHCO₃ (aq, 500 mL) at 0˜10° C. carefully and then extracted with DCM (500 mL). The organic layer was separated and was purified by column chromatogram silica gel (eluent: DCM to DCM/MeOH (v/v)=20/1) to afford the title compound (16.81 g, yield: 60.0%).

### Step 2: tert-butyl (2-(methylcarbamoyl)-5,6-dinitro-2,3-dihydro-1H-inden-2-yl)carbamate

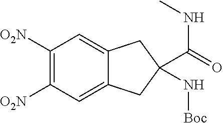 [SMILES: CNC(=O)C1(NC(=O)OC(C)(C)C)Cc2cc([N+](=O)[O-])c([N+](=O)[O-])cc2C1]

The mixture of 2-amino-N-methyl-5,6-dinitro-2,3-dihydro-1H-indene-2-carboxamide (16.81 g, 60 mmol) and Boc₂O (26.16 g) in DCM (200 mL) was stirred overnight at room temperature. The reaction mixture was diluted with PE (200 mL), washed with brine (500 mL), dried over Na₂SO₄, and concentrated in vacuum. The crude product was purified by column chromatography on silica gel (eluent: DCM/MeOH (v/v)=50/1 to 20/1) to afford the tittle product (19.76 g, yield: 86.7%). MS (ESI, m/e) [M+1]⁺ 381.3.

### Step 3: tert-butyl (5,6-diamino-2-(methylcarbamoyl)-2,3-dihydro-1H-inden-2-yl)carbamate

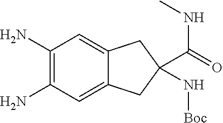 [SMILES: CNC(=O)C1(NC(=O)OC(C)(C)C)Cc2cc(N)c(N)cc2C1]

To the mixture of tert-butyl (2-(methylcarbamoyl)-5,6-dinitro-2,3-dihydro-1H-inden-2-yl)carbamate (19.76 g, 52 mmol) and Pd/C (1.5 g) in MeOH (500 mL) was bubbled with H₂ balloon and stirred overnight under H₂ atmosphere. The reaction mixture was filtered and the filtrate was concentrated in vacuum, then purified by column chromatography on silica gel (eluent: DCM/MeOH (v/v)=50/1 to 20/1) to afford the tittle compound (14.7 g, yield: 88.46%). MS (ESI, m/e) [M+1]⁺ 321.3.

### Step 4: benzyl ((1S)-2-((6-amino-2-((tert-butoxycarbonyl)amino)-2-(methylcarbamoyl)-2,3-dihydro-1H-inden-5-yl)amino)-1-cyclohexyl-2-oxoethyl)carbamate

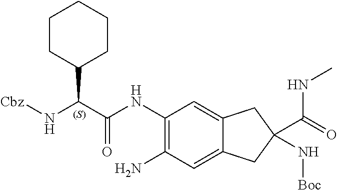

The mixture of (S)-2-(((benzyloxy)carbonyl)amino)-2-cyclohexylacetic acid (13.4 g, 46 mmol), tert-butyl (5,6-diamino-2-(methylcarbamoyl)-2,3-dihydro-1H-inden-2-yl)carbamate (14.70 g, 46 mmol), HATU (19 g, 50 mmol) and TEA (10 g, 100 mmol) in DCM (500 mL) was stirred overnight at room temperature. The reaction mixture was quenched with NaHCO₃ (aq, 500 mL). The organic layer was separated, washed with brine (500 mL), dried over Na₂SO₄, and concentrated in vacuum. The crude product was purified by column chromatography on silica gel (eluent: DCM/MeOH (v/v)=50/1 to 20/1) to afford the tittle compound (9.0 g, yield: 32.95%). MS (ESI, m/e) [M+1]⁺ 594.3.

### Step 5: benzyl ((1S)-(6-((tert-butoxycarbonyl)amino)-6-(methylcarbamoyl)-1,5,6,7-tetrahydroindeno[5,6-d]imidazol-2-yl)(cyclohexyl)methyl)carbamate

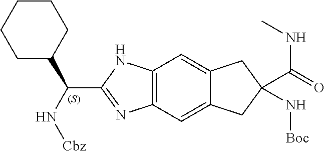

The mixture of benzyl ((1S)-2-((6-amino-2-((tert-butoxycarbonyl)amino)-2-(methylcarbamoyl)-2,3-dihydro-1H-inden-5-yl)amino)-1-cyclohexyl-2-oxoethyl)carbamate (9.0 g) and AcOH (50 mL) was heated to 100° C. and stirred overnight. The reaction mixture was concentrated in vacuum, the residue was diluted with NaHCO₃ (aq, 250 mL), and extracted with DCM (250 mL), washed with brine (500 mL), dried over Na₂SO₄, and concentrated in vacuum. The crude product was purified by column chromatography on silica gel (eluent: DCM/MeOH (v/v)=50/1 to 20/1) to afford the tittle compound (1.63 g, yield: 39.36%). MS (ESI, m/e) [M+1]⁺ 576.3.

### Step 6: benzyl ((1S)-(6-amino-6-(methylcarbamoyl)-1,5,6,7-tetrahydroindeno[5,6-d]imidazol-2-yl)(cyclohexyl)methyl)carbamate

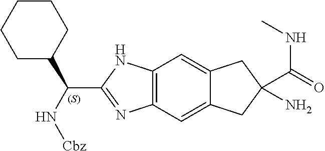

To a solution of benzyl ((1S)-(6-((tert-butoxycarbonyl)amino)-6-(methylcarbamoyl)-1,5,6,7-tetrahydroindeno[5,6-d]imidazol-2-yl)(cyclohexyl)methyl)carbamate (5.0 g, 8.69 mmol) in DCM (100 mL) was added HCl acid (4M, in Dioxane, 100 mL). The mixture was stirred overnight at room temperature. The reaction mixture was concentrated under reduced pressure to remove solvent. The residue was diluted with NaHCO₃ (aq, 250 mL), and extracted with DCM (250 mL). The organic layer was washed with brine (200 mL), dried over Na₂SO₄, and concentrated in vacuum. The crude product was purified by column chromatography on silica gel (eluent: DCM/MeOH (v/v)=50/1 to 20/1) to afford the tittle compound (1.63 g, yield: 39.36%). MS (ESI, m/e) [M+1]⁺ 476.2.

### Step 7: benzyl ((1S)-(6-(((R)-2-((tert-butoxycarbonyl)amino)butyl)amino)-6-(methylcarbamoyl)-1,5,6,7-tetrahydroindeno[5,6-d]imidazol-2-yl)(cyclohexyl)methyl)carbamate

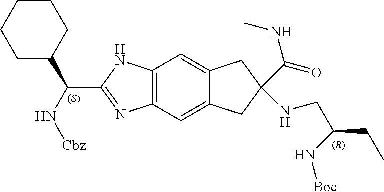

The mixture of benzyl ((1S)-(6-amino-6-(methylcarbamoyl)-1,5,6,7-tetrahydroindeno[5,6-d]imidazol-2-yl)(cyclohexyl)methyl)carbamate (300 mg, 0.63 mmol), tert-butyl (R)-(3-methyl-1-oxobutan-2-yl)carbamate (252 mg, 1.26 mmol) and NaBH(OAc)₃ (267 mg, 1.26 mmol) in DCM (50 mL) was stirred for 2 hrs at room temperature. The reaction mixture was quenched with NaHCO₃ (aq, 100 mL), and extracted with DCM (50 mL). The organic layer was dried over Na₂SO₄ and concentrated in vacuum. The residue was purified by column chromatography on silica gel (eluent: DCM/MeOH (v/v)=50/1). Benzyl ((1S)-(6-(((R)-2-((tert-butoxycarbonyl)amino)butyl)amino)-6-(methylcarbamoyl)-1,5,6,7-tetrahydroindeno[5,6-d]imidazol-2-yl)(cyclohexyl)methyl)carbamate (310 mg, yield: 71.4%) was obtained. MS (ESI, m/e) [M+1]⁺ 647.4.

### Step 8: benzyl ((1S)-(6-(((R)-2-aminobutyl)amino)-6-(methylcarbamoyl)-1,5,6,7-tetrahydroindeno[5,6-d]imidazol-2-yl)(cyclohexyl)methyl)carbamate

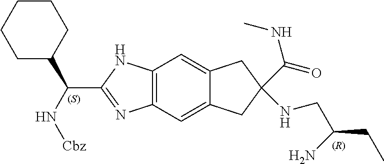

The mixture of benzyl ((1S)-(6-(((R)-2-((tert-butoxycarbonyl)amino)butyl)amino)-6-(methylcarbamoyl)-1,5,6,7-tetrahydroindeno[5,6-d]imidazol-2-yl)(cyclohexyl)methyl)carbamate (310 mg, 0.45 mmol) and TFA (2 mL) in DCM (5 mL) was stirred for 2 hrs. The mixture was concentrated in vacuo to afford benzyl ((1S)-(6-(((R)-2-aminobutyl)amino)-6-(methylcarbamoyl)-1,5,6,7-tetrahydroindeno[5,6-d]imidazol-2-yl)(cyclohexyl)methyl)carbamate (236 mg, crude), which was used directly in next step. MS (ESI, m/e) [M+1]⁺ 547.3.

### Step 9: benzyl ((1S)-cyclohexyl(6-((R)-4-ethyl-2-oxoimidazolidin-1-yl)-6-(methylcarbamoyl)-1,5,6,7-tetrahydroindeno[5,6-d]imidazol-2-yl)methyl)carbamate

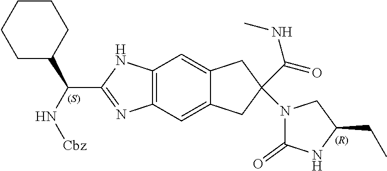

The mixture of benzyl ((1S)-(6-(((R)-2-aminobutyl)amino)-6-(methylcarbamoyl)-1,5,6,7-tetrahydroindeno[5,6-d]imidazol-2-yl)(cyclohexyl)methyl)carbamate (236 mg, 0.44 mmol), TEA (0.5 mL) and CDI (142 mg, 0.88 mmol) in THF (20 mL) was heated to 60° C. and stirred for 2 hrs. The reaction mixture was quenched with brine (50 mL), extracted with EA (50×2 mL). Combined layers were dried over Na₂SO₄ and concentrated in vacuum. The crude product was purified by column chromatography on silica gel (eluent: DCM/MeOH (v/v)=50/1 to 20/1). Benzyl ((1S)-cyclohexyl(6-((R)-4-ethyl-2-oxoimidazolidin-1-yl)-6-(methylcarbamoyl)-1,5,6,7-tetrahydroindeno[5,6-d]imidazol-2-yl)methyl)carbamate was obtained (236 mg, yield: 93.18%). MS (ESI, m/e) [M+1]⁺ 573.3.

### Step 10: 2-((S)-amino(cyclohexyl)methyl)-6-((R)-4-ethyl-2-oxoimidazolidin-1-yl)-N-methyl-1,5,6,7-tetrahydroindeno[5,6-d]imidazole-6-carboxamide

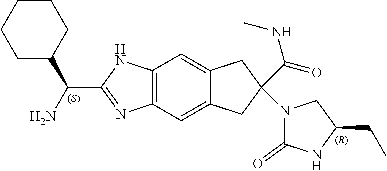

To the mixture of was benzyl ((1S)-cyclohexyl(6-((R)-4-ethyl-2-oxoimidazolidin-1-yl)-6-(methylcarbamoyl)-1,5,6,7-tetrahydroindeno[5,6-d]imidazol-2-yl)methyl)carbamate (236 mg, 0.41 mmol), and Pd/C (20 mg) in MeOH (50 mL) was bubbled with H₂ balloon and stirred overnight at H₂ atmosphere for deprotection. The reaction mixture was then filtrated, and the filtrate was concentrated in vacuum. The crude product was purified by column chromatography on silica gel (eluent: DCM/MeOH (v/v)=50/1 to 20/1) to afford the tittle amine compound (142 mg, yield: 55.66%). MS (ESI, m/e) [M+1]⁺ 439.3.

### Step 11: N-((1S)-cyclohexyl(6-((R)-4-ethyl-2-oxoimidazolidin-1-yl)-6-(methylcarbamoyl)-1,5,6,7-tetrahydroindeno[5,6-d]imidazol-2-yl)methyl)-4-methyl-1,2,5-oxadiazole-3-carboxamide

### Example 32

To a solution of 2-((S)-amino(cyclohexyl)methyl)-6-((R)-4-ethyl-2-oxoimidazolidin-1-yl)-N-methyl-1,5,6,7-tetrahydroindeno[5,6-d]imidazole-6-carboxamide (22 mg, 0.05 mmol) in DCM (10 mL) were added 4-methyl-1,2,5-oxadiazole-3-carboxylic acid (7 mg, 0.05 mmol), HATU (19 mg, 0.05 mmol) and TEA (10 mg, 0.1 mmol). The mixture was stirred at room temperature for 1 hour. The reaction mixture was quenched with aq. NaHCO₃ (50 mL) and diluted with DCM (20 mL). The organic layers were dried over Na₂SO₄, filtered and concentrated in vacuum. The crude product was purified by column chromatography on silica gel (eluent: DCM/MeOH (v/v)=50/1 to 20/1). The tittle compound was obtained (13 mg, yield: 47.4%). ¹H NMR (400 MHz, DMSO-d₆) δ ppm: 12.20 (br, 1H), 9.42 (d, J=8.8 Hz, 1H), 7.55-7.25 (m, 3H), 6.69 (s, 1H), 5.05 (t, J=8.4 Hz, 1H), 3.65-3.40 (m, 4H), 3.32-3.23 (m, 2H), 2.93-2.87 (m, 1H), 2.58 (d, J=4.4 Hz, 3H), 2.10-1.55 (m, 5H), 1.42-1.27 (m, 3H), 1.21-0.95 (m, 5H), 0.71 (t, J=7.2 Hz, 3H). MS (ESI, m/e) [M+1]⁺ 549.4.

### Example 145: N-((1S)-1-cyclohexyl-2-((6-(methylcarbamoyl)-6-(6-oxo-5,7-diazaspiro[2.5]octan-5-yl)-4,5,6,7-tetrahydrobenzo[b]thiophen-2-yl)amino)-2-oxoethyl)-1-methyl-1H-pyrazole-5-carboxamide

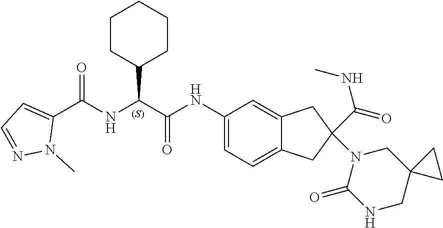

### Step 1: 9,12-dioxa-1,3-diazadispiro[4.2.4⁸.2⁵]tetradecane-2,4-dione

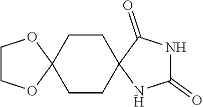 [SMILES: O=C1NC(=O)C2(CCC3(CC2)OCCO3)N1]

A mixture of 1,4-dioxaspiro[4.5]decan-8-one (9.55 g, 61.2 mmol), NaCN (3 g, 61.2 mmol) and (NH₄)₂CO₃ (23.5 g, 244.8 mmol) in H₂O (100 mL) and EtOH (5 mL) was stirred at 60° C. overnight. The reaction mixture was cooled to room temperature and stayed 1 day. The precipitate was filtered and then the cake was dried in air to obtain 9,12-dioxa-1,3-diazadispiro[4.2.4⁸.2⁵]tetradecane-2,4-dione (7.38 g, crude). H NMR (400 MHz, DMSO-d₆) δ ppm: 10.59 (s, 1H), 8.44 (s, 1H), 3.87 (s, 4H), 1.95-1.78 (m, 2H), 1.73-1.69 (m, 4H), 1.59-1.56 (m, 2H).

### Step 2: 8-amino-1,4-dioxaspiro[4.5]decane-8-carboxylic acid

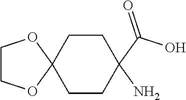 [SMILES: NC1(C(=O)O)CCC2(CC1)OCCO2]

A solution of 9,12-dioxa-1,3-diazadispiro[4.2.4⁸.2⁵]tetradecane-2,4-dione (2 g) in NaOH solution (6 N, 15 mL) was stirred at 140° C. in a sealed tube for 3 hrs. The reaction mixture was cooled to room temperature and acidified to pH=4 with 6N HCl acid. After the mixture was concentrated in vacuum to remove solvent, the residue was dissolved with DCM/MeOH=10/1 (80 mL) and stirred for 10 min. The mixture was filtered and the filtrate was concentrated to obtain 8-amino-1,4-dioxaspiro[4.5]decane-8-carboxylic acid (1.13 g, crude). MS (ESI) m/e [M+1]⁺ 202.1

### Step 3: 8-(((benzyloxy)carbonyl)amino)-1,4-dioxaspiro[4.5]decane-8-carboxylic acid

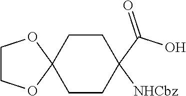 [SMILES: O=C(NC1(C(=O)O)CCC2(CC1)OCCO2)OCc1ccccc1]

A mixture of 8-amino-1,4-dioxaspiro[4.5]decane-8-carboxylic acid (1.13 g), CbzCl (966 mg) and Et₃N (1.15 g, 3.06 mmol) in THF (30 mL) and H₂O (30 mL) was stirred at room temperature for 3 hrs. The reaction mixture was diluted with MTBE (30 mL) and stirred for 10 min. The water phase was collected and concentrated in vacuum. The resulted sloid was dried in air to obtain 8-(((benzyloxy)carbonyl)amino)-1,4-dioxaspiro[4.5]decane-8-carboxylic acid (1.86 g, crude). MS (ESI) m/e [M+1]⁺ 337.1.

### Step 4: benzyl (8-(methylcarbamoyl)-1,4-dioxaspiro[4.5]decan-8-yl)carbamate

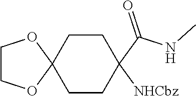 [SMILES: CNC(=O)C1(NC(=O)OCc2ccccc2)CCC2(CC1)OCCO2]

A mixture of 8-(((benzyloxy)carbonyl)amino)-1,4-dioxaspiro[4.5]decane-8-carboxylic acid (1.86 g), methylamine hydrochloride (385 mg, 5.7 mmol), HATU (2.17 g, 5.7 mmol) and Et₃N (1.15 g, 11.4 mmol) in DCM (60 mL) was stirred at room temperature overnight. The reaction mixture was concentrated and the residue was then purified by column chromatography on silica gel (eluent: DCM/MeOH (v/v)=50/1). benzyl (8-(methylcarbamoyl)-1,4-dioxaspiro[4.5]decan-8-yl)carbamate (1.77 g) was obtained. MS (ESI) m/e [M+1]⁺ 349.2.

### Step 5: benzyl (1-(methylcarbamoyl)-4-oxocyclohexyl)carbamate

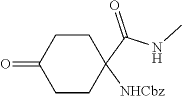 [SMILES: CNC(=O)C1(NC(=O)OCc2ccccc2)CCC(=O)CC1]

A solution of benzyl (8-(methylcarbamoyl)-1,4-dioxaspiro[4.5]decan-8-yl)carbamate (1.77 g, 5.7 mmol) in TFA (10 mL) was stirred at room temperature for 3 hrs. The reaction mixture was concentrated to remove TFA. The residue was neutralized with aq. NaHCO₃ and was then extracted with DCM (50 mL×3). The organic layers were washed with brine, dried over anhydrous Na₂SO₄, filtered and concentrated in vacuum. Benzyl (1-(methylcarbamoyl)-4-oxocyclohexyl)carbamate (615 mg, crude) was obtained. MS (ESI) m/e [M+1]⁺ 305.1

### Step 6: tert-butyl 2-amino-6-(((benzyloxy)carbonyl)amino)-6-(methylcarbamoyl)-4,5,6,7-tetrahydrobenzo[b]thiophene-3-carboxylate

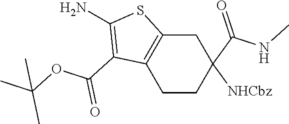 [SMILES: CNC(=O)C1(NC(=O)OCc2ccccc2)CCc2c(sc(N)c2C(=O)OC(C)(C)C)C1]

A mixture of benzyl (1-(methylcarbamoyl)-4-oxocyclohexyl)carbamate (615 mg, 2.02 mmol), tert-butyl 2-cyanoacetate (500 mg, 2.02 mmol), sulfur (65 mg, 2.02 mmol) and morpholine (176 mg, 2.02 mmol) in EtOH (25 mL) was stirred at 80° C. overnight. The mixture was cooled to room temperature and concentrated in vacuum. The residue was purified by pre-TLC (eluent: EA/PE (v/v)=1/1). Tert-butyl 2-amino-6-(((benzyloxy)carbonyl)amino)-6-(methylcarbamoyl)-4,5,6,7-tetrahydrobenzo[b]thiophene-3-carboxylate (287 mg) was obtained. MS (ESI) m/e [M+1]⁺ 460.3.

### Step 7: benzyl (2-amino-6-(methylcarbamoyl)-4,5,6,7-tetrahydrobenzo[b]thiophen-6-yl)carbamate

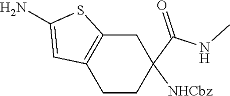 [SMILES: CNC(=O)C1(NC(=O)OCc2ccccc2)CCc2cc(N)sc2C1]

To a solution of tert-butyl 2-amino-6-(((benzyloxy)carbonyl)amino)-6-(methylcarbamoyl)-4,5,6,7-tetrahydrobenzo[b]thiophene-3-carboxylate (287 mg, 0.625 mmol) in THF (30 mL) were added HCl acid (6 N, 10 mL). The mixture was stirred at 60° C. for 4 hrs. After cooled to room temperature, the reaction mixture was neutralized with aq. NaHCO₃ and was then extracted with DCM (50 mL×3). The organic layers were washed with brine, dried over anhydrous Na₂SO₄, filtered and concentrated in vacuum. The residue was purified by pre-TLC (eluent: MeOH/DCM (v/v)=1/10) to obtain benzyl (2-amino-6-(methylcarbamoyl)-4,5,6,7-tetrahydrobenzo[b]thiophen-6-yl)carbamate (237 mg). MS (ESI) m/e [M+1]+360.2.

### Step 8: benzyl (2-((S)-2-((tert-butoxycarbonyl)amino)-2-cyclohexylacetamido)-6-(methylcarbamoyl)-4,5,6,7-tetrahydrobenzo[b]thiophen-6-yl)carbamate

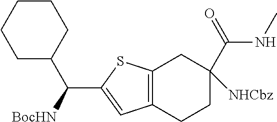 [SMILES: CNC(=O)C1(NC(=O)OCc2ccccc2)CCc2cc([C@@H](NC(=O)OC(C)(C)C)C3CCCCC3)sc2C1]

To a solution of benzyl (2-amino-6-(methylcarbamoyl)-4,5,6,7-tetrahydrobenzo[b]thiophen-6-yl)carbamate (237 mg, 0.625 mmol) in DCM (15 mL) were added (S)-2-((tert-butoxycarbonyl)amino)-2-cyclohexylacetic acid (161 mg, 0.625 mmol), HATU (238 mg, 0.625 mmol) and Et₃N (189 mg, 1.875 mmol). The mixture was stirred at room temperature overnight. The reaction mixture was concentrated, and the residue was purified by pre-TLC (MeOH/DCM=1/10). Benzyl (2-((S)-2-((tert-butoxycarbonyl)amino)-2-cyclohexylacetamido)-6-(methylcarbamoyl)-4,5,6,7-tetrahydrobenzo[b]thiophen-6-yl)carbamate (223 mg) was obtained. MS (ESI) m/e [M+1]⁺ 599.3.

### Step 9: benzyl (2-((S)-2-amino-2-cyclohexylacetamido)-6-(methylcarbamoyl)-4,5,6,7-tetrahydrobenzo[b]thiophen-6-yl)carbamate

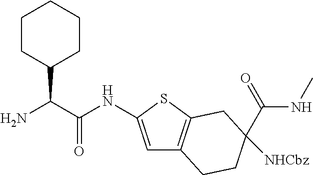 [SMILES: CNC(=O)C1(NC(=O)OCc2ccccc2)CCc2cc(NC(=O)[C@@H](N)C3CCCCC3)sc2C1]

A solution of benzyl (2-((S)-2-((tert-butoxycarbonyl)amino)-2-cyclohexylacetamido)-6-(methylcarbamoyl)-4,5,6,7-tetrahydrobenzo[b]thiophen-6-yl)carbamate (223 mg, 0.372 mmol) in TFA (5 mL) was stirred at room temperature for 1 hr. The reaction was concentrated in vacuum to give the crude title compound (300 mg). MS (ESI) m/e [M+1]⁺ 499.4.

### Step 10: benzyl (2-((S)-2-cyclohexyl-2-(1-methyl-1H-pyrazole-5-carboxamido)acetamido)-6-(methylcarbamoyl)-4,5,6,7-tetrahydrobenzo[b]thiophen-6-yl)carbamate

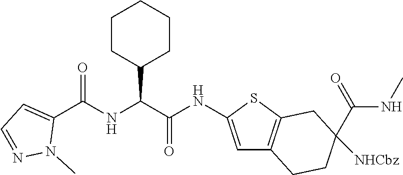 [SMILES: CNC(=O)C1(NC(=O)OCc2ccccc2)CCc2cc(NC(=O)[C@@H](NC(=O)c3ccnn3C)C3CCCCC3)sc2C1]

To a solution of benzyl (2-((S)-2-amino-2-cyclohexylacetamido)-6-(methylcarbamoyl)-4,5,6,7-tetrahydrobenzo[b]thiophen-6-yl)carbamate (300 mg) in DCM (15 mL), were added 1-methyl-1H-pyrazole-5-carboxylic acid (47 mg, 0.372 mmol), HATU (141 mg, 0.372 mmol) and Et₃N (190 mg, 1.86 mmol). The mixture was stirred at room temperature for 48 hrs. The reaction mixture was concentrated in vacuum. The residue was purified by pre-TLC (eluent: MeOH/DCM (v/v)=1/10) to obtain benzyl (2—((S)-2-cyclohexyl-2-(1-methyl-1H-pyrazole-5-carboxamido)acetamido)-6-(methylcarbamoyl)-4,5,6,7-tetrahydrobenzo[b]thiophen-6-yl)carbamate (90 mg). MS (ESI) m/e [M+1]⁺ 607.4.

### Step 11: N-((1S)-2-((6-amino-6-(methylcarbamoyl)-4,5,6,7-tetrahydrobenzo[b]thiophen-2-yl)amino)-1-cyclohexyl-2-oxoethyl)-1-methyl-1H-pyrazole-5-carboxamide

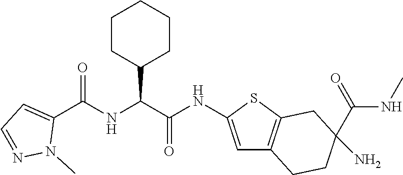 [SMILES: CNC(=O)C1(N)CCc2cc(NC(=O)[C@@H](NC(=O)c3ccnn3C)C3CCCCC3)sc2C1]

To a solution of benzyl (2-((S)-2-cyclohexyl-2-(1-methyl-1H-pyrazole-5-carboxamido)acetamido)-6-(methylcarbamoyl)-4,5,6,7-tetrahydrobenzo[b]thiophen-6-yl)carbamate (80 mg, 0.13 mmol) in DCM (10 mL) was added TMSI (1 mL, 1 mol/L). The mixture was stirred at room temperature for 30 min. The reaction mixture was neutralized with aq. NaHCO₃ and extracted with DCM (50 mL×3). The organic layers were washed with brine, dried over anhydrous Na₂SO₄, filtered and concentrated in vacuum. N-((1S)-2-((6-amino-6-(methylcarbamoyl)-4,5,6,7-tetrahydrobenzo[b]thiophen-2-yl)amino)-1-cyclohexyl-2-oxoethyl)-1-methyl-1H-pyrazole-5-carboxamide (63 mg, crude) was obtained. MS (ESI) m/e [M+1]⁺ 473.2.

### Step 12: tert-butyl ((1-(((2-((S)-2-cyclohexyl-2-(1-methyl-1H-pyrazole-5-carboxamido)acetamido)-6-(methylcarbamoyl)-4,5,6,7-tetrahydrobenzo[b]thiophen-6-yl)amino)methyl)cyclopropyl)methyl)carbamate

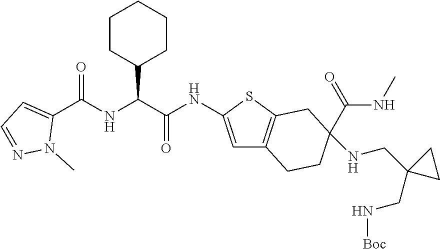 [SMILES: CNC(=O)C1(NCC2(CNC(=O)OC(C)(C)C)CC2)CCc2cc(NC(=O)[C@@H](NC(=O)c3ccnn3C)C3CCCCC3)sc2C1]

A mixture of N-((1S)-2-((6-amino-6-(methylcarbamoyl)-4,5,6,7-tetrahydrobenzo[b]thiophen-2-yl)amino)-1-cyclohexyl-2-oxoethyl)-1-methyl-1H-pyrazole-5-carboxamide (30 mg, 0.063 mmol), tert-butyl ((1-formylcyclopropyl)methyl)carbamate (13 mg, 0.063 mmol) and NaBH(OAc)₃ (14 mg, 0.063 mmol) in DCM (10 mL) was stirred at room temperature overnight. The reaction mixture was concentrated in vacuum. The residue was purified by pre-TLC (eluent: MeOH/DCM (v/v)=1/15) to obtain the desired product (10 mg). MS (ESI) m/e [M+1]⁺ 656.5.

### Step 13: N-((1S)-2-((6-(((1-(aminomethyl)cyclopropyl)methyl)amino)-6-(methylcarbamoyl)-4,5,6,7-tetrahydrobenzo[b]thiophen-2-yl)amino)-1-cyclohexyl-2-oxoethyl)-1-methyl-1H-pyrazole-5-carboxamide

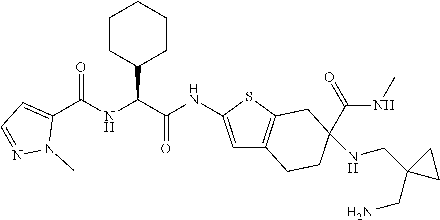 [SMILES: CNC(=O)C1(NCC2(CN)CC2)CCc2cc(NC(=O)[C@@H](NC(=O)c3ccnn3C)C3CCCCC3)sc2C1]

A mixture of tert-butyl ((1-(((2-((S)-2-cyclohexyl-2-(1-methyl-1H-pyrazole-5-carboxamido)acetamido)-6-(methylcarbamoyl)-4,5,6,7-tetrahydrobenzo[b]thiophen-6-yl)amino)methyl)cyclopropyl)methyl)carbamate (10 mg) in TFA (2 mL) was stirred at room temperature for 30 min. The reaction was concentrated to give the desired product as a white solid (10 mg). MS (ESI) m/e [M+1]⁺ 556.4.

### Step 14: N-((1S)-1-cyclohexyl-2-((6-(methylcarbamoyl)-6-(6-oxo-5,7-diazaspiro[2.5]octan-5-yl)-4,5,6,7-tetrahydrobenzo[b]thiophen-2-yl)amino)-2-oxoethyl)-1-methyl-1H-pyrazole-5-carboxamide

### Example 145

To a solution of N-((1S)-2-((6-(((1-(aminomethyl)cyclopropyl)methyl)amino)-6-(methylcarbamoyl)-4,5,6,7-tetrahydrobenzo[b]thiophen-2-yl)amino)-1-cyclohexyl-2-oxoethyl)-1-methyl-1H-pyrazole-5-carboxamide (10 mg) and Et₃N (30 mg) in DMF (2 mL), was added CDI (3 mg). The mixture was stirred at 60° C. for 2 hrs. The reaction mixture was concentrated in vacuum and the residue was purified by pre-TLC (eluent: MeOH/DCM (v/v)=1/15). The title compound example 145 was obtained (2 mg). MS (ESI) m/e [M+1]⁺ 582.2.

### Example 170: N-((1S)-1-cyclohexyl-2-((2-isopropyl-2-(6-oxo-5,7-diazaspiro[2.5]octan-5-yl)-2,3-dihydro-1H-inden-5-yl)amino)-2-oxoethyl)-1-methyl-1H-pyrazole-5-carboxamide

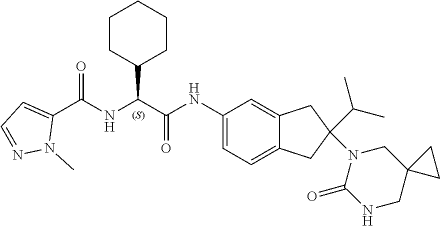

### Step 1: methyl 6-bromo-2-isopropyl-1-oxo-2,3-dihydro-1H-indene-2-carboxylate

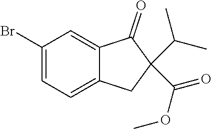 [SMILES: COC(=O)C1(C(C)C)Cc2ccc(Br)cc2C1=O]

To a solution of methyl 6-bromo-1-oxo-2,3-dihydro-1H-indene-2-carboxylate (27 g) in dry DMF (250 ml) was added sodium hydride (8 g of a 60% suspension in oil, 200 mmol) in portions over 20 minutes under N₂ protection. The mixture was stirred for 45 minutes. 2-iodopropane (34 g, 200 mmol) was added dropwise over 15 minutes and the mixture was then stirred for 18 hrs at room temperature. The reaction mixture was concentrated under reduced pressure and the residue was partitioned between EA (150 mL) and HCl acid (2N, 50 mL). The organic layer was separated, dried and concentrated in vacuum. The crude product was purified by column chromatography on silica gel (eluent: PE/EA (v/v)=10/1). Methyl 6-bromo-2-isopropyl-1-oxo-2,3-dihydro-1H-indene-2-carboxylate (10 g, yield: 33%) was obtained. MS (ESI, m/e) [M+1]⁺ 311.0/313.0.

### Step 2: methyl 6-((diphenylmethylene)amino)-2-isopropyl-1-oxo-2,3-dihydro-1H-indene-2-carboxylate

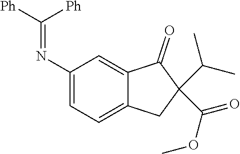 [SMILES: COC(=O)C1(C(C)C)Cc2ccc(N=C(c3ccccc3)c3ccccc3)cc2C1=O]

A mixture of Methyl 6-bromo-2-isopropyl-1-oxo-2,3-dihydro-1H-indene-2-carboxylate (3.11 g, 10 mmol), diphenylmethanimine (2.7 g, 15 mmol), BINAP (1.87 g, 0.3 mmol), Pd₂(dba)₃ (1.4 g, 0.15 mmol) and Cs₂CO₃ (10 g, 30 mmol) in toluene (50 ml) was degassed with N₂. The mixture was heated to 100° C. and stirred for 12 hrs under N₂ atmosphere. The reaction mixture was cooled to room temperature and washed with H₂O, brine. The organic layer was dried over MgSO₄, filtered and concentrated in vacuum. The residue was purified by column chromatography on silica gel (eluent: hexane/EtOAc (v/v)=10/1). Methyl 6-((diphenylmethylene)amino)-2-isopropyl-1-oxo-2,3-dihydro-1H-indene-2-carboxylate (2.2 g, yield: 53.5%) was obtained. MS (ESI, m/e) [M+1]⁺ 412.1

### Step 3: methyl 6-amino-2-isopropyl-1-oxo-2,3-dihydro-1H-indene-2-carboxylate

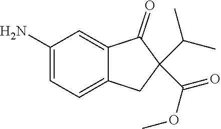 [SMILES: COC(=O)C1(C(C)C)Cc2ccc(N)cc2C1=O]

methyl 6-((diphenylmethylene)amino)-2-isopropyl-1-oxo-2,3-dihydro-1H-indene-2-carboxylate (2.2 g) was dissolved into HCl acid (1N, 15 mL) and stirred for lhour. The reaction mixture was basified with aq. NaHCO₃ to pH=8-9 and extracted with EtOAc (30 mL×3). The combined organic layers were washed with H₂O, brine, dried over MgSO₄, filtered and concentrated in vacuum. The residue was purified by column chromatography on silica gel (eluent: hexane/EtOAc (v/v)=1/1). Methyl 6-amino-2-isopropyl-1-oxo-2,3-dihydro-1H-indene-2-carboxylate (1 g, yield: 76.9%) was obtained. MS (ESI, m/e) [M+1]⁺ 248.1.

### Step 4: methyl 6-amino-1-hydroxy-2-isopropyl-2,3-dihydro-1H-indene-2-carboxylate

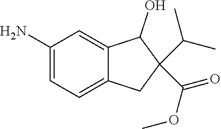 [SMILES: COC(=O)C1(C(C)C)Cc2ccc(N)cc2C1O]

A solution of methyl 6-amino-2-isopropyl-1-oxo-2,3-dihydro-1H-indene-2-carboxylate (1 g, 4 mmol) in MeOH (50 mL) was added NaBH₄ (154 mg, 12 mmol) in portions at 0° C. The reaction mixture was stirred for 1 hour and was then concentrated under reduced pressure. The residue was partitioned between ethyl acetate and water. The organic layer was dried over MgSO₄ and concentrated in vacuum. Methyl 6-amino-1-hydroxy-2-isopropyl-2,3-dihydro-1H-indene-2-carboxylate (1 g, crude) was obtained. MS (ESI, m/e) [M+1]⁺ 250.1.

### Step 5: methyl 5-amino-2-isopropyl-2,3-dihydro-1H-indene-2-carboxylate

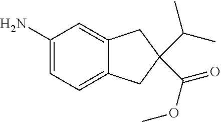 [SMILES: COC(=O)C1(C(C)C)Cc2ccc(N)cc2C1]

To a solution of methyl 6-amino-1-hydroxy-2-isopropyl-2,3-dihydro-1H-indene-2-carboxylate (249 mg, 1 mmol) in TFA (10 mL) was added triethyl silane (2 mL). The mixture was stirred at room temperature for 1 hour. After the reaction mixture was concentrated to remove TFA, the residue was dissolved in DCM (25 mL) and washed with sat·aq·NaHCO₃. The organic layer was dried over MgSO₄ and concentrated in vacuum. Methyl 5-amino-2-isopropyl-2,3-dihydro-1H-indene-2-carboxylate (240 mg, crude) was obtained. MS (ESI, m/e) [M+1]⁺ 234.1.

### Step 6: methyl 5-((S)-2-((tert-butoxycarbonyl)amino)-2-cyclohexylacetamido)-2-isopropyl-2,3-dihydro-1H-indene-2-carboxylate

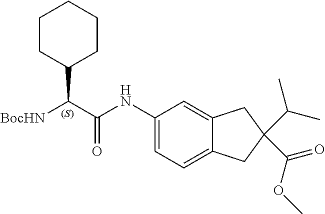

To a solution of methyl 5-amino-2-isopropyl-2,3-dihydro-1H-indene-2-carboxylate (466 mg, 2 mmol) in DCM (25 mL) were added (S)-2-((tert-butoxycarbonyl)amino)-2-cyclohexylacetic acid (819 mg, 3 mmol), HATU (1.35 g, 3.6 mmol) and TEA (1 mL). The mixture was stirred at room temperature for 1 hour. The reaction mixture was concentrated to remove solvent and the residue was purified by prep-TLC (eluent: DCM/MeOH (v/v)=15:1). methyl 5-((S)-2-((tert-butoxycarbonyl)amino)-2-cyclohexylacetamido)-2-isopropyl-2,3-dihydro-1H-indene-2-carboxylate (623 mg, yield: 56%) was obtained. MS (ESI, m/e) [M+1]⁺ 473.3.

### Step 7: methyl 5-((S)-2-amino-2-cyclohexylacetamido)-2-isopropyl-2,3-dihydro-1H-indene-2-carboxylate

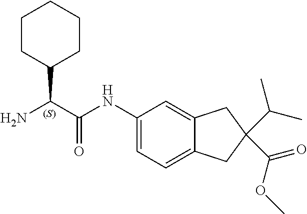

Methyl 5-((S)-2-((tert-butoxycarbonyl)amino)-2-cyclohexylacetamido)-2-isopropyl-2,3-dihydro-1H-indene-2-carboxylate (623 mg, 1.32 mmol) was dissolved into HCl solution (10 mL, 2M in dioxane) and stirred at room temperature for 1 hour. After the reaction mixture was concentrated to remove solvent in vacuum, methyl 5-((S)-2-amino-2-cyclohexylacetamido)-2-isopropyl-2,3-dihydro-1H-indene-2-carboxylate salts of HCl (491 mg, crude) was obtained. MS (ESI, m/e) [M+1]⁺ 373.1.

### Step 8: methyl 5-((S)-2-cyclohexyl-2-(1-methyl-1H-pyrazole-5-carboxamido)acetamido)-2-isopropyl-2,3-dihydro-1H-indene-2-carboxylate

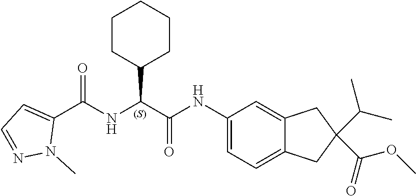

To a solution of methyl 5-((S)-2-amino-2-cyclohexylacetamido)-2-isopropyl-2,3-dihydro-1H-indene-2-carboxylate (491 mg, crude) and TEA (1.0 mL) in DCM (25 mL) were added 1-methyl-1H-pyrazole-5-carboxylic acid (200 mg, 1.58 mmol) and HATU (752 mg, 1.98 mmol). The mixture was stirred at room temperature for 2 hrs. The reaction mixture was quenched with aq. NaHCO₃ (50 mL) and diluted with DCM (20 mL). The organic layers were dried over Na₂SO₄ and concentrated in vacuum. The residue was purified by prep-TLC (eluent: DCM/MeOH (v/v)=15:1). Methyl 5-((S)-2-cyclohexyl-2-(1-methyl-1H-pyrazole-5-carboxamido)acetamido)-2-isopropyl-2,3-dihydro-1H-indene-2-carboxylate (200 mg, yield: 32%) was obtained. MS (ESI, m/e) [M+1]⁺ 481.1.

### Step 9: 5-((S)-2-cyclohexyl-2-(1-methyl-1H-pyrazole-5-carboxamido)acetamido)-2-isopropyl-2,3-dihydro-1H-indene-2-carboxylic acid

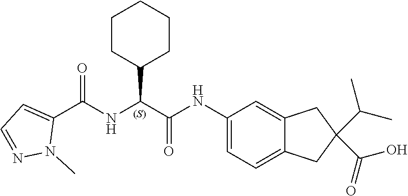

To a solution of methyl 5-((S)-2-cyclohexyl-2-(1-methyl-1H-pyrazole-5-carboxamido)acetamido)-2-isopropyl-2,3-dihydro-1H-indene-2-carboxylate (200 mg, 0.42 mmol) in methanol (5 mL), was added aq. NaOH (2 mL, 1.0 N). The mixture was stirred overnight at room temperature. The reaction mixture was acidified to pH=5-6 with diluted HCl acid and extracted with EtOAc (15 mL×3). The organic layers were washed with H₂O, brine, dried over MgSO₄, filtered and concentrated in vacuum. 5-((S)-2-cyclohexyl-2-(1-methyl-1H-pyrazole-5-carboxamido)acetamido)-2-isopropyl-2,3-dihydro-1H-indene-2-carboxylic acid (100 mg, crude) was obtained. MS (ESI, m/e) [M+1]+467.0.

### Step 10: N-((1S)-2-((2-amino-2-isopropyl-2,3-dihydro-1H-inden-5-yl)amino)-1-cyclohexyl-2-oxoethyl)-1-methyl-1H-pyrazole-5-carboxamide

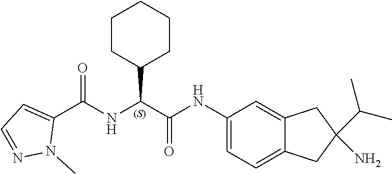

To a solution of 5-((S)-2-cyclohexyl-2-(1-methyl-1H-pyrazole-5-carboxamido)acetamido)-2-isopropyl-2,3-dihydro-1H-indene-2-carboxylic acid (60 mg, 0.13 mmol) in toluene (5 mL) was added DPPA (53 mg) and TEA (0.1 mL). The mixture was heated to 90° C. and stirred for 1 hour. The reaction mixture was cooled and concentrated to remove solvent. The residue was dissolved into HCl acid (5 mL, 1N) and then stirred for 1 hour. The mixture was basified with aq. NaHCO₃ to pH ˜9 and was extracted with EtOAc (10 mL×3). The organic layer was washed with H₂O, brine, dried over MgSO₄, filtered and concentrated in vacuum. The residue was purified by column chromatography on silica gel (eluent: hexane/EtOAc (v/v)=1/1). N-((1S)-2-((2-amino-2-isopropyl-2,3-dihydro-1H-inden-5-yl)amino)-1-cyclohexyl-2-oxoethyl)-1-methyl-1H-pyrazole-5-carboxamide (30 mg, yield: 54%) was obtained. MS (ESI, m/e) [M+1]⁺ 438.1.

### Step 11: tert-butyl ((1-(((5-((S)-2-cyclohexyl-2-(1-methyl-1H-pyrazole-5-carboxamido)acetamido)-2-isopropyl-2,3-dihydro-1H-inden-2-yl)amino)methyl)cyclopropyl)methyl)carbamate

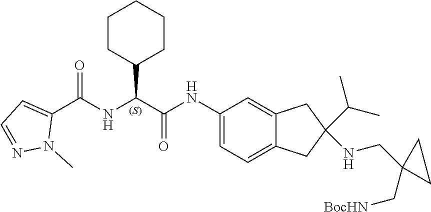

A mixture of N-((1S)-2-((2-amino-2-isopropyl-2,3-dihydro-1H-inden-5-yl)amino)-1-cyclohexyl-2-oxoethyl)-1-methyl-1H-pyrazole-5-carboxamide (30 mg, 0.069 mmol), tert-butyl ((1-formylcyclopropyl)methyl)carbamate (14 mg, 0.069 mmol) and NaBH(OAc)₃ (50 mg) in DCM (5 mL) was stirred at room temperature for 1 hour. The reaction mixture was diluted with DCM (15 mL) and then washed with H₂O, brine. The organic layer was separated and dried over MgSO₄, filtered and concentrated in vacuum. The residue was purified by prep-TLC (eluent: DCM/MeOH (v/v)=15:1). Tert-butyl ((1-(((5-((S)-2-cyclohexyl-2-(1-methyl-1H-pyrazole-5-carboxamido)acetamido)-2-isopropyl-2,3-dihydro-1H-inden-2-yl)amino)methyl)cyclopropyl)methyl)carbamate (20 mg, yield: 47%) was obtained. MS (ESI, m/e) [M+1]⁺ 621.1.

### Step 12: N-((1S)-2-((2-(((1-(aminomethyl)cyclopropyl)methyl)amino)-2-isopropyl-2,3-dihydro-1H-inden-5-yl)amino)-1-cyclohexyl-2-oxoethyl)-1-methyl-1H-pyrazole-5-carboxamide

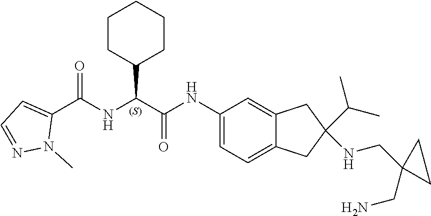

Tert-butyl ((1-(((5-((S)-2-cyclohexyl-2-(1-methyl-1H-pyrazole-5-carboxamido)acetamido)-2-isopropyl-2,3-dihydro-1H-inden-2-yl)amino)methyl)cyclopropyl)methyl)carbamate (20 mg) was dissolved into HCl solution (10 mL, 2M in dioxane) and stirred at room temperature for 1 hour. The reaction mixture was concentrated in vacuum, N-((1S)-2-((2-(((1-(aminomethyl)cyclopropyl)methyl)amino)-2-isopropyl-2,3-dihydro-1H-inden-5-yl)amino)-1-cyclohexyl-2-oxoethyl)-1-methyl-1H-pyrazole-5-carboxamide salts of HCl (20 mg crude) was obtained. MS (ESI, m/e) [M+1]⁺ 521.3.

### Step 13: N-((1S)-1-cyclohexyl-2-((2-isopropyl-2-(6-oxo-5,7-diazaspiro[2.5]octan-5-yl)-2,3-dihydro-1H-inden-5-yl)amino)-2-oxoethyl)-1-methyl-1H-pyrazole-5-carboxamide (Example 170)

To a solution of methyl N-((1S)-2-((2-(((1-(aminomethyl)cyclopropyl)methyl)amino)-2-isopropyl-2,3-dihydro-1H-inden-5-yl)amino)-1-cyclohexyl-2-oxoethyl)-1-methyl-1H-pyrazole-5-carboxamide (20 mg) and TEA (0.5 mL) in THF (5 mL) was added CDI (20 mg). The mixture was heated to reflux and stirred for 3 hrs. The reaction mixture was cooled to room temperature and concentrated in vacuum. The residue was purified by prep-TLC (eluent: DCM/MeOH (v/v)=15:1). The title compound (10 mg, yield: 28.6% for 2 steps) was obtained. ¹H NMR (400 MHz, DMSO-d₆) δ ppm: 10.05 (s, 1H), 8.45 (d, J=8.0 Hz, 1H), 7.45 (t, J=8.6 Hz, 2H), 7.29 (s, 1H), 7.04 (d, J=10.4 Hz, 2H), 6.05 (s, 1H), 4.44-4.31 (m, 1H), 4.03 (s, 3H), 3.21-2.92 (m, 4H), 2.86 (s, 2H), 2.67 (s, 2H), 1.94-1.49 (m, 8H), 128-1.15 (m, 4H), 0.70 (d, J=6.6 Hz, 6H), 0.52-0.38 (m, 4H). MS (ESI, m/e) [M+1]⁺ 547.7.

### Example 212: N-((2S)-1,1-dicyclopropyl-3-((2-isopropyl-2-(2-oxo-4-(trifluoromethyl)imidazolidin-1-yl)-2,3-dihydro-1H-inden-5-yl)amino)-3-oxopropan-2-yl)-4-methyl-1,2,5-oxadiazole-3-carboxamide

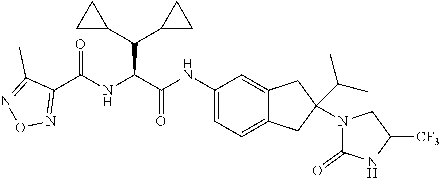 [SMILES: Cc1nonc1C(=O)N[C@H](C(=O)Nc1ccc2c(c1)CC(C(C)C)(N1CC(C(F)(F)F)NC1=O)C2)C(C1CC1)C1CC1]

### Step 1: methyl 2-isopropyl-1-oxo-2,3-dihydro-1H-indene-2-carboxylate

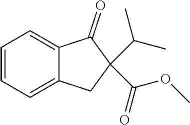 [SMILES: COC(=O)C1(C(C)C)Cc2ccccc2C1=O]

To a solution of methyl 1-oxo-2,3-dihydro-1H-indene-2-carboxylate (14 g, 73.6 mmol) in dry DMF (250 ml) was added sodium hydride (8.8 g of a 60% suspension in oil, 222 mmol) in portions over 20 minutes at 0° C. After the mixture was further stirred for 45 minutes, 2-iodopropane (25 g, 148 mmol) was added dropwise over 15 minutes. The mixture was then heated to 90° C. and and stirred for 18 hrs. The reaction mixture was cooled to r.t and then poured into ice/water (100 mL), extracted with EA (100 mL×3). The combined organic layers were dried over anhydrous Na₂SO₄, filtered and concentrated in vacuum. The residue was purified by column chromatography on silica gel (eluent: PE/EA (v/v)=10/1). Methyl 2-isopropyl-1-oxo-2,3-dihydro-1H-indene-2-carboxylate (8 g, 50%) was obtained. MS (ESI, m/e) [M+1]⁺ 233.1.

### Step 2: methyl 1-hydroxy-2-isopropyl-2,3-dihydro-1H-indene-2-carboxylate

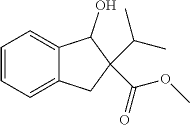 [SMILES: COC(=O)C1(C(C)C)Cc2ccccc2C1O]

To a solution of methyl 2-isopropyl-1-oxo-2,3-dihydro-1H-indene-2-carboxylate (1.8 g, 7.8 mmol) in MeOH (50 mL) was added NaBH₄ (1.2 g, 32 mmol) in portions at 0° C. The mixture was stirred for 1 hour and then was concentrated under reduced pressure. The residue was partitioned between ethyl acetate and water. The organic layer was dried over MgSO₄, filtered and concentrated in vacuum. Methyl 1-hydroxy-2-isopropyl-2,3-dihydro-1H-indene-2-carboxylate (1.8 g, crude) was obtained, which was used directly in next step. MS (ESI, m/e) [M−17]⁺ 217.1.

### Step 3: methyl 2-isopropyl-2,3-dihydro-1H-indene-2-carboxylate

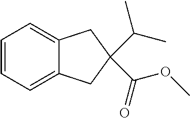 [SMILES: COC(=O)C1(C(C)C)Cc2ccccc2C1]

To a solution of methyl 1-hydroxy-2-isopropyl-2,3-dihydro-1H-indene-2-carboxylate (1.7 g, 7.2 mmol) in TFA (25 mL) was added triethyl silane (5 mL). The mixture was stirred at room temperature for 1 hour. The reaction mixture was then concentrated under reduced pressure to remove solvent. The residue was dissolved in DCM (25 mL) and washed with sat·aq·NaHCO₃ (10 mL). The organic layer was washed with brine, dried over MgSO₄ and concentrated in vacuum. Methyl 2-isopropyl-2,3-dihydro-1H-indene-2-carboxylate (1.5 g, crude) was obtained, which was used directly in next step. MS (ESI, m/e) [M+1]⁺ 219.1.

### Step 4: 2-isopropyl-2,3-dihydro-1H-indene-2-carboxylic acid

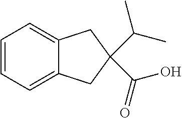

To a solution of methyl 2-isopropyl-2,3-dihydro-1H-indene-2-carboxylate (218 mg, 1 mmol) in methanol (3 mL) was added aq. NaOH (1.0 N, 2 mL, 2 mmol). The mixture was stirred overnight at room temperature. The reaction mixture was then acidified with HCl acid (2.0 N), and extracted with EtOAc (10 mL×3). The combined organic layers were washed with brine, H₂O, dried over MgSO₄, filtered and concentrated in vacuum. 2-isopropyl-2,3-dihydro-1H-indene-2-carboxylic acid (200 mg, crude) was obtained. MS (ESI, m/e) [M+1]⁺ 205.1.

### Step 5: 2-isopropyl-2,3-dihydro-1H-inden-2-amine

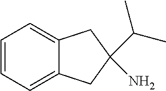

To a solution of 2-isopropyl-2,3-dihydro-1H-indene-2-carboxylic acid (1.2 g, 6.8 mmol) in toluene (25 mL) was added DPPA (2.75 g) and TEA (3 mL). The mixture was heated to 90° C. and stirred for 1 hour. The reaction mixture was concentrated under reduced pressure. The residue was diluted into HCl acid (1N, 15 mL) and then stirred for 1 hour. The mixture was basified with aq. NaHCO₃ to pH ˜8-9 and extracted with EtOAc (10 mL×3). The combined organic layers were washed with H₂O, brine, dried over MgSO₄, filtered and concentrated in vacuum. The residue was purified by column chromatography on silica gel (eluent: hexane/EtOAc (v/v)=4/1). 2-isopropyl-2,3-dihydro-1H-inden-2-amine (700 mg, yield: 69%) was obtained. MS (ESI, m/e) [M+1]⁺ 176.1

### Step 6: tert-butyl (1,1,1-trifluoro-3-((2-isopropyl-2,3-dihydro-1H-inden-2-yl)amino)-3-oxopropan-2-yl)carbamate

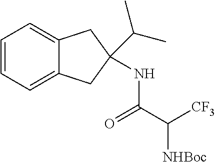 [SMILES: CC(C)C1(NC(=O)C(NC(=O)OC(C)(C)C)C(F)(F)F)Cc2ccccc2C1]

To the mixture of 2-(((benzyloxy)carbonyl)amino)-3,3,3-trifluoropropanoic acid (1.17 g, 4.8 mmol), HATU (1.98 g, 5.2 mmol) in DCM (30 mL) were added 2-isopropyl-2,3-dihydro-1H-inden-2-aminen (700 mg, 4 mmol) and TEA (1.6 g, 15.4 mmol) at 0° C. The mixture was stirred for 1.5 hrs at room temperature. The reaction mixture was diluted with DCM and quenched with aq. NaHCO₃. The organic layer was washed with brine, dried and concentrated in vacuum. The residue was purified by column chromatography on silica gel (eluent: hexane/EtOAc (v/v)=5/1). Tert-butyl (1,1,1-trifluoro-3-((2-isopropyl-2,3-dihydro-1H-inden-2-yl)amino)-3-oxopropan-2-yl)carbamate (750 mg, yield: 46%) was obtained. MS (ESI, m/e) [M+1]⁺ 401.1.

### Step 7: tert-butyl (1,1,1-trifluoro-3-((2-isopropyl-2,3-dihydro-1H-inden-2-yl)amino)propan-2-yl)carbamate

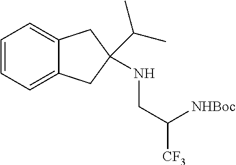 [SMILES: CC(C)C1(NCC(NC(=O)OC(C)(C)C)C(F)(F)F)Cc2ccccc2C1]

To a solution of tert-butyl (1,1,1-trifluoro-3-((2-isopropyl-2,3-dihydro-1H-inden-2-yl)amino)-3-oxopropan-2-yl)carbamate (750 mg, 1.8 mmol) in THF (10 mL) was added BH₃-Me₂S (10 mL) dropwise. The mixture was heated to 70° C. and stirred for 4 hrs. The reaction mixture was quenched with MeOH (10 mL). After solvent was removed, the residue was purified by column chromatography on silica gel (eluent: hexane/EtOAc (v/v)=4/1). Tert-butyl (1,1,1-trifluoro-3-((2-isopropyl-2,3-dihydro-1H-inden-2-yl)amino)propan-2-yl)carbamate (500 mg, 72%) was obtained. MS (ESI, m/e) [M+1]⁺ 387.2

### Step 8: 3,3,3-trifluoro-N¹-(2-isopropyl-2,3-dihydro-1H-inden-2-yl)propane-1,2-diamine

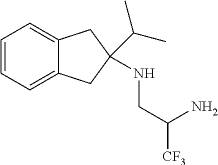 [SMILES: CC(C)C1(NCC(N)C(F)(F)F)Cc2ccccc2C1]

To a solution of tert-butyl (1,1,1-trifluoro-3-((2-isopropyl-2,3-dihydro-1H-inden-2-yl)amino)propan-2-yl)carbamate (500 mg) in THF was added HCl in dioxane (2M, 10 mL). The mixture was stirred at room temperature for 1 hour. After the solvent was removed in vacuum, 3,3,3-trifluoro-N¹-(2-isopropyl-2,3-dihydro-1H-inden-2-yl)propane-1,2-diamine salts (450 mg, crude) was obtained, which was used directly in next step.

### Step 9: 1-(2-isopropyl-2,3-dihydro-1H-inden-2-yl)-4-(trifluoromethyl)imidazolidin-2-one

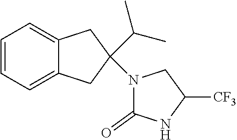 [SMILES: CC(C)C1(N2CC(C(F)(F)F)NC2=O)Cc2ccccc2C1]

To a solution of 3,3,3-trifluoro-N1-(2-isopropyl-2,3-dihydro-1H-inden-2-yl)propane-1,2-diamine (400 mg, 1.4 mmol) and TEA (1.5 mL) in THF (15 mL) was added CDI (324 mg, 2 mmol). The mixture was heated to reflux and stirred for 3 hrs. After cooled to r.t., the reaction mixture was concentrated under reduced pressure. The residue was purified by column chromatography on silica gel (eluent: hexane/EtOAc (v/v)=5/1). 1-(2-isopropyl-2,3-dihydro-1H-inden-2-yl)-4-(trifluoromethyl)imidazolidin-2-one (150 mg, 2 steps yield: 34%) was obtained. MS (ESI, m/e) [M+1]+313.0.

### Step 10: 1-(2-isopropyl-5-nitro-2,3-dihydro-1H-inden-2-yl)-4-(trifluoromethyl)imidazolidin-2-one

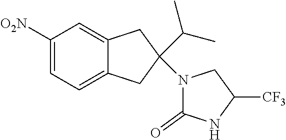 [SMILES: CC(C)C1(N2CC(C(F)(F)F)NC2=O)Cc2ccc([N+](=O)[O-])cc2C1]

To the mixture of H₂SO₄ (2 mL) and HNO₃ (3 mL) was added a solution of 1-(2-isopropyl-2,3-dihydro-1H-inden-2-yl)-4-(trifluoromethyl)imidazolidin-2-one (80 mg, 0.25 mmol) in HOAc (2 mL) in batches at 0° C. The mixture was stirred for 5 mins at 0° C. and then quenched with saturated aq. NaHCO₃ (10 mL), extracted with DCM (10 mL×3). The organic layers were washed with H₂O, brine, dried over MgSO₄, filtered, and concentrated in vacuum. The residue was purified by column chromatography on silica gel (eluent: hexane/EtOAc (v/v)=4/1). 1-(2-isopropyl-5-nitro-2,3-dihydro-1H-inden-2-yl)-4-(trifluoromethyl)imidazolidin-2-one (40 mg, yield: 44%) was obtained. MS (ESI, m/e) [M+1]⁺ 358.0.

### Step 11: 1-(5-amino-2-isopropyl-2,3-dihydro-1H-inden-2-yl)-4-(trifluoromethyl)imidazolidin-2-one

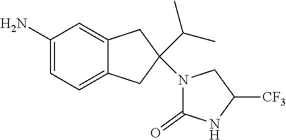 [SMILES: CC(C)C1(N2CC(C(F)(F)F)NC2=O)Cc2ccc(N)cc2C1]

To a solution of 1-(2-isopropyl-5-nitro-2,3-dihydro-1H-inden-2-yl)-4-(trifluoromethyl)imidazolidin-2-one (40 mg, 0.11 mmol) in MeOH (10 mL) was added Pd/C (10 mg). The mixture was stirred under H₂ atmosphere for 1 hour. The reaction mixture was filtered and the filtrate was concentrated in vacuum. 1-(5-amino-2-isopropyl-2,3-dihydro-1H-inden-2-yl)-4-(trifluoromethyl)imidazolidin-2-one (40 mg, crude) was obtained. MS (ESI, m/e) [M+1]⁺ 328.0.

### Step 12: tert-butyl ((2S)-1,1-dicyclopropyl-3-((2-isopropyl-2-(2-oxo-4-(trifluoromethyl)imidazolidin-1-yl)-2,3-dihydro-1H-inden-5-yl)amino)-3-oxopropan-2-yl)carbamate

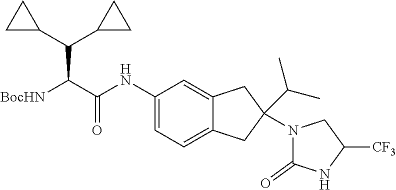 [SMILES: CC(C)C1(N2CC(C(F)(F)F)NC2=O)Cc2ccc(NC(=O)[C@@H](NC(=O)OC(C)(C)C)C(C3CC3)C3CC3)cc2C1]

To a solution of 1-(5-amino-2-isopropyl-2,3-dihydro-1H-inden-2-yl)-4-(trifluoromethyl)imidazolidin-2-one (40 mg, 0.11 mmol) in DCM were added (S)-2-((tert-butoxycarbonyl)amino)-3,3-dicyclopropylpropanoic acid (30 mg, 0.11 mmol), HATU (63 mg, 0.17 mmol) and TEA (0.1 mL). The mixture was stirred at room temperature for 1 hour. The reaction mixture was concentrated and purified by prep-TLC to give tert-butyl ((2S)-1,1-dicyclopropyl-3-((2-isopropyl-2-(2-oxo-4-(trifluoromethyl)imidazolidin-1-yl)-2,3-dihydro-1H-inden-5-yl)amino)-3-oxopropan-2-yl)carbamate (30 mg, yield: 47%). MS (ESI, m/e) [M+1]⁺ 579.1.

### Step 13: (2S)-2-amino-3,3-dicyclopropyl-N-(2-isopropyl-2-(2-oxo-4-(trifluoromethyl)imidazolidin-1-yl)-2,3-dihydro-1H-inden-5-yl)propanamide

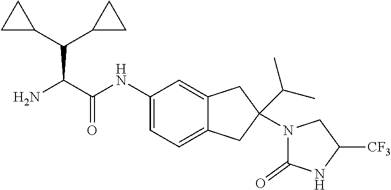 [SMILES: CC(C)C1(N2CC(C(F)(F)F)NC2=O)Cc2ccc(NC(=O)[C@@H](N)C(C3CC3)C3CC3)cc2C1]

To a solution of tert-butyl ((2S)-1,1-dicyclopropyl-3-((2-isopropyl-2-(2-oxo-4-(trifluoromethyl)imidazolidin-1-yl)-2,3-dihydro-1H-inden-5-yl)amino)-3-oxopropan-2-yl)carbamate (30 mg) in THF was added HCl in dioxane (2M, 10 mL). The mixture was stirred at room temperature for 1 hour. After the solvent was removed in vacuum, (2S)-2-amino-3,3-dicyclopropyl-N-(2-isopropyl-2-(2-oxo-4-(trifluoromethyl)imidazolidin-1-yl)-2,3-dihydro-1H-inden-5-yl)propanamide (20 mg, crude) was obtained. MS (ESI, m/e) [M+1]⁺ 479.1.

### Step 14: N-((2S)-1,1-dicyclopropyl-3-((2-isopropyl-2-(2-oxo-4-(trifluoromethyl)imidazolidin-1-yl)-2,3-dihydro-1H-inden-5-yl)amino)-3-oxopropan-2-yl)-4-methyl-1,2,5-oxadiazole-3-carboxamide

### Example 212

To a solution of (2S)-2-amino-3,3-dicyclopropyl-N-(2-isopropyl-2-(2-oxo-4-(trifluoromethyl)imidazolidin-1-yl)-2,3-dihydro-1H-inden-5-yl)propanamide (20 mg, 0.042 mmol) in DCM (10 mL) were added 4-methyl-1,2,5-oxadiazole-3-carboxylic acid (5 mg, 0.042 mmol), HATU (24 mg, 0.063 mmol) and TEA (0.1 mL). The mixture was stirred at room temperature for 1 hour. The reaction mixture was concentrated to remove solvent. The residue was purified by prep-TLC (eluent: DCM/CH3OH (v/v)=20/1). The title compound example 212 (8 mg, yield: 32%) was obtained. ¹H NMR (400 MHz, DMSO-d₆) δ ppm: 10.06 (s, 1H), 9.02 (d, J=8.8 Hz, 1H), 7.46 (d, J=7.8 Hz, 1H), 7.33 (d, J=8.2 Hz, 1H), 7.22 (s, 1H), 7.12 (d, J=8.0 Hz, 1H), 4.89-4.80 (m, 1H), 4.24 (s, 1H), 3.65 (d, J=10.6 Hz, 1H), 3.49-3.41 (m, 2H), 3.09-3.01 (m, 3H), 2.59-2.53 (m, 1H), 2.48 (s, 3H), 0.94-0.76 (m, 9H), 0.50-0.11 (m, 8H). MS (ESI, m/e) [M+1]⁺ 589.5.

### Example 238

### N-((2S)-1,1-dicyclopropyl-3-((2-((R)-4-isopropyl-2-oxoimidazolidin-1-yl)-2-(2,2,2-trifluoro-1-hydroxyethyl)-2,3-dihydro-1H-inden-5-yl)amino)-3-oxopropan-2-yl)-4-methyl-1,2,5-oxadiazole-3-carboxamide

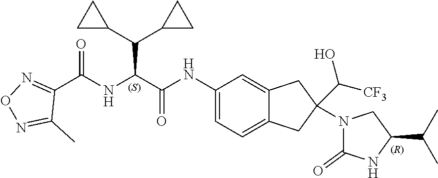

### Step 1: (R)-1-(2-(hydroxymethyl)-2,3-dihydro-1H-inden-2-yl)-4-isopropylimidazolidin-2-one

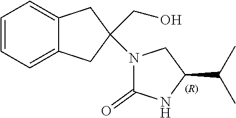

To a solution of methyl (R)-2-(4-isopropyl-2-oxoimidazolidin-1-yl)-2,3-dihydro-1H-indene-2-carboxylate (430 mg, 1.4 mmol, prepared by the procedure similar with 2-((R)-4-isopropyl-2-oxoimidazolidin-1-yl)-N-methyl-5-nitro-2,3-dihydro-1H-indene-2-carboxamide as described in intermediate A2) in THF (8 mL) was added a solution of LAH (2.5 M, 1.5 mL, 3.7 mmol) dropwise with stirring at 0° C. under N₂ atmosphere. After addition, the mixture was stirred for 1 hour at 0° C. The reaction mixture was quenched by aq. Na₂SO₄ and extracted with EA (15 mL×3). The combined organic layers were washed with brine, dried over anhydrous Na₂SO₄, filtered and concentrated in vacuum. The residue was purified by Prep-TLC (eluent: DCM/MeOH (v/v)=20/1) to afford (R)-1-(2-(hydroxymethyl)-2,3-dihydro-1H-inden-2-yl)-4-isopropylimidazolidin-2-one (270 mg, yield: 69%). MS (ESI) m/e [M+1]⁺ 275.1.

### Step 2: (R)-2-(4-isopropyl-2-oxoimidazolidin-1-yl)-2,3-dihydro-1H-indene-2-carbaldehyde

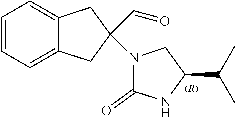

To a solution of (R)-1-(2-(hydroxymethyl)-2,3-dihydro-1H-inden-2-yl)-4-isopropylimidazolidin-2-one (270 mg, 1.0 mmol) in DCM (20 mL). The resulting solution was cooled to 0° C. under nitrogen atmosphere was added Dess-Martin reagent (636 mg, 1.5 mmol) in portions at 0° C. under nitrogen atmosphere. The mixture was stirred at room temperature for 2 hrs. The reaction mixture was quenched by aq. Na₂S₂O₃ (5 mL) at 0° C. and was then diluted with aq. NaHCO₃ (10 mL) and extracted with EA (15 mL×3). The combined organic layers were washed with brine, dried and concentrated in vacuum. The residue was purified by Prep-TLC (eluent: DCM/MeOH (v/v)=30/1) to afford (R)-2-(4-isopropyl-2-oxoimidazolidin-1-yl)-2,3-dihydro-1H-indene-2-carbaldehyde (240 mg, yield: 88%). MS (ESI) m/e [M+1]⁺ 273.1.

### Step 3: (4R)-4-isopropyl-1-(2-(2,2,2-trifluoro-1-((trimethylsilyl)oxy)ethyl)-2,3-dihydro-1H-inden-2-yl)imidazolidin-2-one

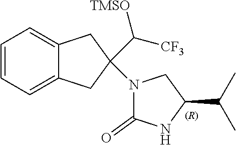

To a solution of (R)-2-(4-isopropyl-2-oxoimidazolidin-1-yl)-2,3-dihydro-1H-indene-2-carbaldehyde (240 mg, 0.88 mmol) in THF (10 mL) was added CsF (174 mg, 1.15 mmol) under nitrogen atmosphere. The resulting mixture was cooled to 0° C. To this mixture was added TMS-CF₃ (163 mg, 1.15 mmol). The mixture was stirred at room temperature for 30 min. The reaction mixture was diluted with EtOAc (50 mL) and then washed with brine. The organic layer was separated, dried over Na₂SO₄ and concentrated in vacuum. (4R)-4-isopropyl-1-(2-(2,2,2-trifluoro-1-((trimethylsilyl)oxy)ethyl)-2,3-dihydro-1H-inden-2-yl)imidazolidin-2-one (300 mg, crude) was obtained, which used directly in next step.

### Step 4: (4R)-4-isopropyl-1-(2-(2,2,2-trifluoro-1-hydroxyethyl)-2,3-dihydro-1H-inden-2-yl)imidazolidin-2-one

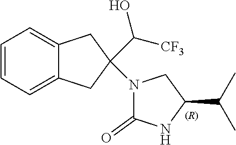

(4R)-4-isopropyl-1-(2-(2,2,2-trifluoro-1-((trimethylsilyl)oxy)ethyl)-2,3-dihydro-1H-inden-2-yl)imidazolidin-2-one (300 mg, crude) was dissolved into THF (10 mL) and was cooled to 0° C. To this solution was added TBAF (0.1 mL, 1M). The mixture was stirred at 0° C. for 1 hour. The reaction mixture was diluted with EtOAc (50 mL) and then washed with brine. The organic layer was separated, dried over Na₂SO₄ and concentrated in vacuum. The residue was purified by Prep-TLC (eluent: DCM/MeOH (v/v)=25/1) to afford (4R)-4-isopropyl-1-(2-(2,2,2-trifluoro-1-hydroxyethyl)-2,3-dihydro-1H-inden-2-yl)imidazolidin-2-one (60 mg, yield: 20% for two steps). MS (ESI) m/e [M+1]⁺ 343.2.

### Step 5: (4R)-1-(5-amino-2-(2,2,2-trifluoro-1-hydroxyethyl)-2,3-dihydro-1H-inden-2-yl)-4-isopropylimidazolidin-2-one

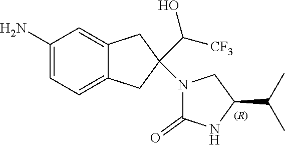

To the mixture of H₂SO₄ (1 mL) and HNO₃ (1.5 mL) was added a solution of (4R)-4-isopropyl-1-(2-(2,2,2-trifluoro-1-hydroxyethyl)-2,3-dihydro-1H-inden-2-yl)imidazolidin-2-one (60 mg) in AcOH (2 mL) in batches at −5° C. The mixture was further stirred for 5 min at −5° C. The reaction mixture was poured in water/ice and was then extracted with EtOAc (15 mL×3). The combined organic layers were washed with water, aq. NaHCO₃ and brine, dried and concentrated in vacuum. (4R)-1-(5-amino-2-(2,2,2-trifluoro-1-hydroxyethyl)-2,3-dihydro-1H-inden-2-yl)-4-isopropylimidazolidin-2-one (70 mg, crude) was afforded. The crude nitro product was dissolved in MeOH (10 mL), stirred and hydrogenated with H₂ in presence of Pd/C (35 mg) for 1 hour at room temperature. (4R)-1-(5-amino-2-(2,2,2-trifluoro-1-hydroxyethyl)-2,3-dihydro-1H-inden-2-yl)-4-isopropylimidazolidin-2-one (40 mg, crude) was obtained. MS (ESI) m/e [M+1]+358.3.

### Step 6: benzyl ((2S)-1,1-dicyclopropyl-3-((2-((R)-4-isopropyl-2-oxoimidazolidin-1-yl)-2-(2,2,2-trifluoro-1-hydroxyethyl)-2,3-dihydro-1H-inden-5-yl)amino)-3-oxopropan-2-yl)carbamate

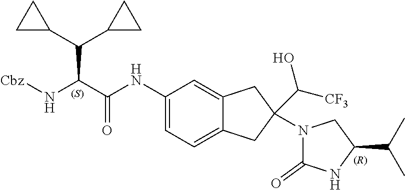

To a solution of (4R)-1-(5-amino-2-(2,2,2-trifluoro-1-hydroxyethyl)-2,3-dihydro-1H-inden-2-yl)-4-isopropylimidazolidin-2-one (40 mg, crude) in DCM (5 mL) was added (S)-2-(((benzyloxy)carbonyl)amino)-3,3-dicyclopropylpropanoic acid (34 mg, 0.11 mmol), DIPEA (43.4 mg, 0.34 mmol) and HATU (55.4 mg, 0.15 mmol). The mixture was stirred at room temperature for 1 hour. The reaction mixture was diluted with DCM (15 mL) and then washed with aq. NaHCO₃. The organic layer was washed with brine, dried and concentrated in vacuum. The crude product was purified by Prep-TLC (eluent: EA/PE (v/v)=1/1). Benzyl ((2S)-1,1-dicyclopropyl-3-((2-((R)-4-isopropyl-2-oxoimidazolidin-1-yl)-2-(2,2,2-trifluoro-1-hydroxyethyl)-2,3-dihydro-1H-inden-5-yl)amino)-3-oxopropan-2-yl)carbamate (30 mg, yield: 42%) was obtained. MS (ESI) m/e [M+1]⁺ 643.5.

### Step 7: (2S)-2-amino-3,3-dicyclopropyl-N-(2-((R)-4-isopropyl-2-oxoimidazolidin-1-yl)-2-(2,2,2-trifluoro-1-hydroxyethyl)-2,3-dihydro-1H-inden-5-yl)propanamide

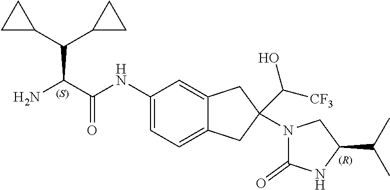

A solution of benzyl ((2S)-1,1-dicyclopropyl-3-((2-((R)-4-isopropyl-2-oxoimidazolidin-1-yl)-2-(2,2,2-trifluoro-1-hydroxyethyl)-2,3-dihydro-1H-inden-5-yl)amino)-3-oxopropan-2-yl)carbamate (30 mg, 0.05 mmol) in MeOH was added Pd/C (30 mg). This mixture was then degassed with H₂ and stirred at room temperature for 1 hour under H₂ atmosphere. The reaction mixture was diluted with DCM and filtered. The filtrate was concentrated to afford (2S)-2-amino-3,3-dicyclopropyl-N-(2-((R)-4-isopropyl-2-oxoimidazolidin-1-yl)-2-(2,2,2-trifluoro-1-hydroxyethyl)-2,3-dihydro-1H-inden-5-yl)propanamide (29 mg, crude). MS (ESI) m/e [M+1]⁺ 509.4.

### Step 8: N-((2S)-1,1-dicyclopropyl-3-((2-((R)-4-isopropyl-2-oxoimidazolidin-1-yl)-2-(2,2,2-trifluoro-1-hydroxyethyl)-2,3-dihydro-1H-inden-5-yl)amino)-3-oxopropan-2-yl)-4-methyl-1,2,5-oxadiazole-3-carboxamide (Example 238)

To a solution of (2S)-2-amino-3,3-dicyclopropyl-N-(2-((R)-4-isopropyl-2-oxoimidazolidin-1-yl)-2-(2,2,2-trifluoro-1-hydroxyethyl)-2,3-dihydro-1H-inden-5-yl)propanamide (29 mg, crude) in DCM (5 mL) was added 4-methyl-1,2,5-oxadiazole-3-carboxylic acid (7.3 mg, 0.06 mmol), DIPEA (22 mg, 0.17 mmol) and HATU (33 mg, 0.09 mmol). The mixture was stirred at room temperature for 1 hour. The reaction mixture was diluted with DCM (15 mL) and then washed with aq. NaHCO₃. The organic layer was washed with brine, dried and concentrated in vacuum. The residue was firstly purified by prep-TLC. The crude product was then purified by Prep-HPLC (mobile phase: MeCN/water (0.1% FA); column: SunFire; elution: 60%-80% MeCN). The title compound example 238 (8.16 mg, 28% for two steps) was obtained. ¹H NMR (400 MHz, DMSO-d₆) δ ppm: 10.12 (s, 1H), 9.05 (d, J=8.8 Hz, 1H), 7.54-7.50 (m, 1H), 7.43-7.32 (m, 1H), 7.24-7.11 (m, 1H), 6.90-6.76 (m, 1H), 4.87-4.75 (m, 2H), 3.53-3.38 (m, 3H), 3.31-3.14 (m, 4H), 2.97-2.89 (m, 1H), 2.49 (s, 3H), 1.37-1.24 (m, 1H), 0.93-0.67 (m, 3H), 0.63-0.50 (m, 6H), 0.49-0.35 (m, 2H), 0.33-0.08 (m, 6H). MS (ESI) m/e [M+1]⁺ 619.5.

### Example 256: N-((2S)-1,1-dicyclopropyl-3-oxo-3-((2-(((S)-2-oxo-4-(trifluoromethyl)imidazolidin-1-yl)methyl)-2,3-dihydrobenzofuran-6-yl)amino)propan-2-yl)-1-isopropyl-1H-pyrazole-5-carboxamide

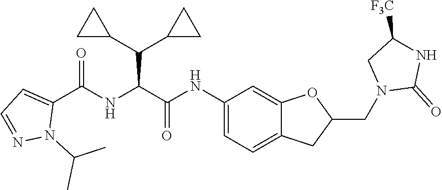 [SMILES: CC(C)n1nccc1C(=O)N[C@H](C(=O)Nc1ccc2c(c1)OC(CN1C[C@@H](C(F)(F)F)NC1=O)C2)C(C1CC1)C1CC1]

### Step 1: 2-(2,2-dimethoxyethoxy)-4-nitrobenzaldehyde

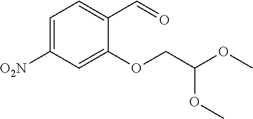 [SMILES: COC(COc1cc([N+](=O)[O-])ccc1C=O)OC]

To a solution of 2-hydroxy-4-nitrobenzaldehyde (10 g, 29.92 mmol) in DMA (100 mL) was added 2-bromo-1,1-dimethoxyethane (40 g, 239.35 mmol), K₂CO₃ (33 g, 239.35 mmol) at room temperature slowly. The mixture was stirred at 140° C. for 12 hrs. The reaction mixture was poured into ice-water (500 mL). The precipitate was collected by filtration and washed with water (500 mL). After the cake was dried in vacuum, 2-(2,2-dimethoxyethoxy)-4-nitrobenzaldehyde (10 g, yield: 66%) was obtained. ¹H NMR (400 MHz, CDCl₃) δ ppm 10.55 (s, 1H), 8.04-7.96 (m, 1H), 7.94-7.86 (m, 2H), 4.81 (t, J=5.2 Hz, 1H), 4.23 (d, J=5.2 Hz, 2H), 3.52-3.44 (m, 6H).

### Step 2: 6-nitrobenzofuran-2-carbaldehyde

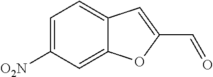 [SMILES: O=Cc1cc2ccc([N+](=O)[O-])cc2o1]

A solution of 2-(2,2-dimethoxyethoxy)-4-nitrobenzaldehyde (10 g, 39.18 mmol) in AcOH (100 mL) was heated to 60° C. and stirred for 12 hrs. The reaction mixture was concentrated under reduced pressure. The residue was poured into saturated aq. Na₂CO₃ solution (100 mL) and then extracted with DCM (100 mL×3). The combined organic layers were washed with brine, dried over anhydrous Na₂SO₄, filtered and concentrated in vacuum. 6-nitrobenzofuran-2-carbaldehyde (6 g, yield: 80%) was obtained. ¹H NMR (400 MHz, CDCl₃) δ ppm 9.99 (s, 1H), 8.52 (s, 1H), 8.28 (dd, J=2.0, 8.8 Hz, 1H), 7.92 (d, J=8.8 Hz, 1H), 7.65 (d, J=0.4 Hz, 1H).

### Step 3: (S)-3,3,3-trifluoro-N¹-((6-nitrobenzofuran-2-yl)methyl)propane-1,2-diamine

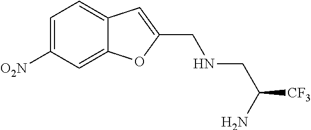 [SMILES: N[C@@H](CNCc1cc2ccc([N+](=O)[O-])cc2o1)C(F)(F)F]

To a solution of 6-nitrobenzofuran-2-carbaldehyde (2 g, 10.46 mmol) in i-PrOH (40 mL) was added (S)-3,3,3-trifluoropropane-1,2-diamine hydrochloride (2.58 g, 15.70 mmol) and TEA (4.24 g, 41.85 mmol) at 20° C. The mixture was stirred at 60° C. for 2 hrs. After TLC indicated the aldehyde reactant was consumed completely, AcOH (3.14 g, 52.30 mmol) and NaBH₃CN (1.64 g, 26.16 mmol) were added into the reaction mixture (mainly containing the imine intermediate of (S)-1,1,1-trifluoro-3-(((6-nitrobenzofuran-2-yl)methylene)amino)propan-2-amine in i-PrOH) at 0° C. After addition, the reaction mixture was further stirred at 80° C. for 12 hours. The reaction mixture was cooled and then poured into saturated aq. NaHCO₃ solution (20 mL) and extracted with DCM (50 mL×3). The combined layers were washed with brine, dried over anhydrous Na₂SO₄, filtered and concentrated in vacuum. The residue was purified by column chromatography on silica gel (eluent: Petroleum ether/Ethyl acetate (v/v)=100/1 to 0/1). (S)-3,3,3-trifluoro-N¹-((6-nitrobenzofuran-2-yl)methyl)propane-1,2-diamine (2.3 g, 70% yield) was obtained. ¹H NMR (400 MHz, CDCl₃) δ ppm 8.37 (d, J=1.2 Hz, 1H), 8.18 (dd, J=2.0, 8.4 Hz, 1H), 7.63 (d, J=8.4 Hz, 1H), 6.74 (s, 1H), 4.05 (s, 2H), 3.36 (s, 1H), 3.37 (s, 1H), 3.00 (dd, J=3.6, 12.4 Hz, 1H), 2.75 (dd, J=8.8, 12.0 Hz, 1H). MS (ESI, m/e) [M+1]⁺ 303.2.

### Step 4: (S)-1-((6-nitrobenzofuran-2-yl)methyl)-4-(trifluoromethyl)imidazolidin-2-one

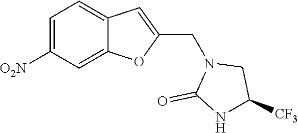 [SMILES: O=C1N[C@H](C(F)(F)F)CN1Cc1cc2ccc([N+](=O)[O-])cc2o1]

To a solution of (S)-3,3,3-trifluoro-N¹-((6-nitrobenzofuran-2-yl)methyl)propane-1,2-diamine (2.3 g, 7.58 mmol) in THF (20 mL) was added CDI (1.84 g, 11.38 mmol) at room temperature. The mixture was stirred at 80° C. for 2 hrs. The reaction mixture was cooled and poured into water (20 mL), extracted with DCM (20 mL×3). The combined organic layers were washed with brine, dried over anhydrous Na₂SO₄, filtered and concentrated in vacuum. The crude product was purified by column chromatography on silica gel (eluent: Petroleum ether/Ethyl acetate (v/v)=50/1 to 1/1). (S)-1-((6-nitrobenzofuran-2-yl)methyl)-4-(trifluoromethyl)imidazolidin-2-one (1.5 g, yield: 60%) was obtained. ¹H NMR (400 MHz, CDCl₃) δ ppm 8.42-8.35 (m, 1H), 8.23-8.15 (m, 1H), 7.67-7.62 (m, 1H), 6.81-6.75 (m, 1H), 5.01-4.94 (m, 1H), 4.68-4.54 (m, 1H), 4.24-4.17 (m, 1H), 3.84-3.62 (m, 2H).

### Step 5: (4S)-1-((6-amino-2,3-dihydrobenzofuran-2-yl)methyl)-4-(trifluoromethyl)imidazolidin-2-one

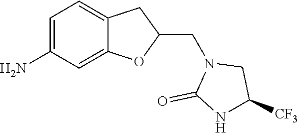 [SMILES: Nc1ccc2c(c1)OC(CN1C[C@@H](C(F)(F)F)NC1=O)C2]

To a solution of (S)-1-((6-nitrobenzofuran-2-yl)methyl)-4-(trifluoromethyl)imidazolidin-2-one (1.5 g, 4.56 mmol) in i-PrOH (10 mL) was added Pd/C (180 mg) at room temperature. The mixture was stirred and hydrogenated under H₂ atmosphere (50 psi) at 50° C. for 12 hrs. The reaction mixture was filtered and concentrated in vacuum. The crude product was purified by column chromatography on silica gel (eluent: Petroleum ether/Ethyl acetate (v/v)=100/1 to 0/1). (4S)-1-((6-amino-2,3-dihydrobenzofuran-2-yl)methyl)-4-(trifluoromethyl)imidazolidin-2-one (810 mg, yield: 59%) was obtained. ¹H NMR (400 MHz, CDCl₃) δ ppm 6.92 (d, J=7.6 Hz, 1H), 6.30-6.11 (m, 2H), 5.16-4.88 (m, 2H), 4.18-4.07 (m, 1 H), 3.84-3.71 (m, 1H), 3.67-3.54 (m, 3H), 3.47-3.29 (m, 1H), 3.18 (ddd, J=3.2, 9.6, 15.2 Hz, 1H), 2.86 (dt, J=7.2, 16.4 Hz, 1H). MS (ESI, m/e) [M+1]⁺ 302.1.

### Step 6: benzyl ((2S)-1,1-dicyclopropyl-3-oxo-3-((2-(((S)-2-oxo-4-(trifluoromethyl)imidazolidin-1-yl)methyl)-2,3-dihydrobenzofuran-6-yl)amino)propan-2-yl)carbamate

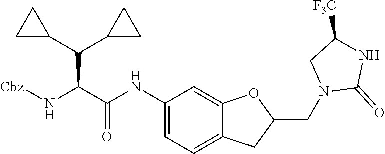 [SMILES: O=C(N[C@H](C(=O)Nc1ccc2c(c1)OC(CN1C[C@@H](C(F)(F)F)NC1=O)C2)C(C1CC1)C1CC1)OCc1ccccc1]

To a solution of (4S)-1-((6-amino-2,3-dihydrobenzofuran-2-yl)methyl)-4-(trifluoromethyl)imidazolidin-2-one (301 mg, 1.0 mmol) in DCM (100 mL) were added (S)-2-(((benzyloxy)carbonyl)amino)-3,3-dicyclopropylpropanoic acid (303 mg, 1.0 mmol), HATU (380 mg, 1.0 mmol) and TEA (202 mg, 2.0 mmol). The mixture was stirred at room temperature for 4 hrs. The reaction mixture was then quenched with NaHCO₃ (aq, 150 mL). The organic layer was separated, washed with brine, dried over anhydrous Na₂SO₄, filtered and concentrated in vacuum. The crude product was purified by column chromatography on silica gel (eluent: DCM/MeOH (v/v)=50/1). benzyl ((2S)-1,1-dicyclopropyl-3-oxo-3-((2-(((S)-2-oxo-4-(trifluoromethyl)imidazolidin-1-yl)methyl)-2,3-dihydrobenzofuran-6-yl)amino)propan-2-yl)carbamate (469 mg, 80%) was obtained. MS (ESI, m/e) [M+1]⁺ 587.2.

### Step 7: (2S)-2-amino-3,3-dicyclopropyl-N-(2-(((S)-2-oxo-4-(trifluoromethyl)imidazolidin-1-yl)methyl)-2,3-dihydrobenzofuran-6-yl)propanamide

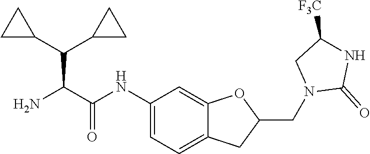 [SMILES: N[C@H](C(=O)Nc1ccc2c(c1)OC(CN1C[C@@H](C(F)(F)F)NC1=O)C2)C(C1CC1)C1CC1]

To the mixture of benzyl ((2S)-1,1-dicyclopropyl-3-oxo-3-((2-(((S)-2-oxo-4-(trifluoromethyl)imidazolidin-1-yl)methyl)-2,3-dihydrobenzofuran-6-yl)amino)propan-2-yl)carbamate (469 mg, 0.8 mmol) and Pd/C (50 mg) in MeOH (50 mL) was bubbled with H₂ balloon and stirred for 2 hrs. The mixture was filtrated and concentrated in vacuum. The crude product (2S)-2-amino-3,3-dicyclopropyl-N-(2-(((S)-2-oxo-4-(trifluoromethyl)imidazolidin-1-yl)methyl)-2,3-dihydrobenzofuran-6-yl)propanamide (226 mg, crude) was obtained, which was used in next step directly. MS (ESI, m/e) [M+1]⁺ 453.2.

### Step 8: N-((2S)-1,1-dicyclopropyl-3-oxo-3-((2-(((S)-2-oxo-4-(trifluoromethyl)imidazolidin-1-yl)methyl)-2,3-dihydrobenzofuran-6-yl)amino)propan-2-yl)-1-isopropyl-1H-pyrazole-5-carboxamide

### Example 256

To a solution of (2S)-2-amino-3,3-dicyclopropyl-N-(2-(((S)-2-oxo-4-(trifluoromethyl)imidazolidin-1-yl)methyl)-2,3-dihydrobenzofuran-6-yl)propanamide (226 mg, 0.5 mmol) in DCM (50 mL) were added 1-isopropyl-1H-pyrazole-5-carboxylic acid (14 mg, 0.09 mmol), HATU (40 mg, 0.09 mmol) and DIEA (23 mg, 0.18 mmol). The mixture was stirred at room temperature for 4 hrs. The reaction mixture was quenched with saturated aq. NaHCO₃ (50 mL). The organic layer was separated, washed with brine, dried over anhydrous Na₂SO₄, filtered and concentrated in vacuum. The residue was purified by Prep-TLC (eluent: MeOH/DCM (v/v)=1/20) to give the crude product. The crude product was purified by prep-HPLC. N-((2S)-1,1-dicyclopropyl-3-oxo-3-((2-(((S)-2-oxo-4-(trifluoromethyl)imidazolidin-1-yl)methyl)-2,3-dihydrobenzofuran-6-yl)amino)propan-2-yl)-1-isopropyl-1H-pyrazole-5-carboxamide (16.85 mg, 32.3%) was obtained. ¹H NMR (400 MHz, DMSO-d₆) δ ppm: 10.09 (d, J=5.0 Hz, 1H), 8.42 (d, J=8.7 Hz, 1H), 7.52-7.50 (m, 2H), 7.18 (d, J=4.4 Hz, 1H), 7.10 (t, J=8.4 Hz, 1H), 7.00 (d, J=7.8 Hz, 1H), 6.92 (s, 1H), 5.44-5.37 (m, 1H), 4.95-4.93 (m, 1H), 4.76 (t, J=8.2 Hz, 1H), 4.35 (s, 1H), 3.79-3.66 (m, 1H), 3.58-3.37 (m, 2H), 3.30-3.14 (m, 2H), 2.87-2.82 (m, 1H), 1.38-1.33 (m, 6H), 0.89-0.67 (m, 3H), 0.45-0.06 (m, 8H). MS (ESI, m/e) [M+1]⁺ 589.5.

### Example 261: N-((2S)-1,1-dicyclopropyl-3-((2-((R)-4-isopropyl-2-oxoimidazolidin-1-yl)-2-(o-tolylcarbamoyl)-2,3-dihydro-1H-inden-5-yl)amino)-3-oxopropan-2-yl)-4-methyl-1,2,5-oxadiazole-3-carboxamide

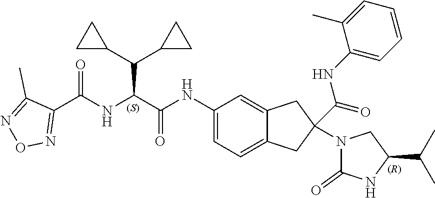

To a solution of 5-((S)-2-amino-3,3-dicyclopropylpropanamido)-2-((R)-4-isopropyl-2-oxoimidazolidin-1-yl)-N-(o-tolyl)-2,3-dihydro-1H-indene-2-carboxamide (65 mg, 0.12 mmol) in DCM (5 mL) was added 4-methyl-1,2,5-oxadiazole-3-carboxylic acid (23 mg, 0.18 mmol), HATU (68 mg, 0.18 mmol) and DIEA (31 mg, 0.24 mmol) at 0° C. The mixture was stirred at room temperature for 12 hrs. The reaction mixture was diluted with water (3 mL), extracted with DCM (5 mL×3). The combined organic layers were washed with brine, dried over anhydrous Na₂SO₄, filtered and concentrated in vacuum. The crude product was purified by prep-HPLC (column: Phenomenex Luna C18 75*30 mm*3 um; mobile phase: [water(NH4HCO3)-ACN]; B %: 40%-70%, 8 min). N-((2S)-1,1-dicyclopropyl-3-((2-((R)-4-isopropyl-2-oxoimidazolidin-1-yl)-2-(o-tolylcarbamoyl)-2,3-dihydro-1H-inden-5-yl)amino)-3-oxopropan-2-yl)-4-methyl-1,2,5-oxadiazole-3-carboxamide (15.5 mg, yield: 19.8%) was obtained. ¹H NMR (400 MHz, DMSO-d₆) δ ppm 10.10 (s, 1H), 9.07-8.96 (m, 2H), 7.56-7.47 (m, 1H), 7.46-7.32 (m, 2H), 7.19 (d, J=7.6 Hz, 2H), 7.16-7.10 (m, 1H), 7.08-7.03 (m, 1H), 7.02 (s, 1H), 4.86 (t, J=8.0 Hz, 1H), 3.70 (t, J=17.6 Hz, 1H), 3.62-3.49 (m, 2H), 3.48-3.38 (m, 1H), 3.30-3.25 (m, 1H), 3.22 (d, J=16.4 Hz, 1H), 2.94 (td, J=7.6, 12.4 Hz, 1H), 2.49 (s, 3H), 2.17 (s, 3H), 1.55-1.42 (m, 1H), 0.93-0.84 (m, 1H), 0.82-0.68 (m, 8H), 0.52-0.36 (m, 2H), 0.34-0.11 (m, 6H). MS (ESI, m/e) [M+1]⁺ 654.2.

### Example 279: N-((2S)-1,1-dicyclopropyl-3-((2-(2-hydroxypropan-2-yl)-2-(2-oxo-4-(trifluoromethyl)imidazolidin-1-yl)-2,3-dihydro-1H-inden-5-yl)amino)-3-oxopropan-2-yl)-4-methyl-1,2,5-oxadiazole-3-carboxamide

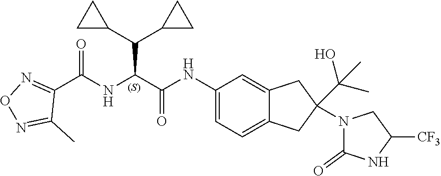

### Step 1: 2-(2-amino-2,3-dihydro-1H-inden-2-yl)propan-2-ol

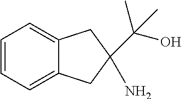

Methyl 2-amino-2,3-dihydro-1H-indene-2-carboxylate (1 g, 5.2 mmol) was dissolved into THF (20 mL) and cooled to 0° C. under N₂ atmosphere. To this solution was added CH₃MgBr (26.2 mL, 26.2 mmol) dropwise with stirring below 10° C. The mixture was warmed to room temperature and stirred for 30 min. The reaction mixture was quenched with aq. NH₄Cl at 0° C. and was extracted with DCM (30 mL×3). The combined organic layers were washed with brine, dried and concentrated in vacuum. The residue was purified by column chromatography on silica gel (eluent: DCM/MeOH (v/v)=10/1). 2-(2-amino-2,3-dihydro-1H-inden-2-yl)propan-2-ol (980 mg, yield: 98%) was obtained. MS (ESI) m/e [M+1]⁺ 192.2.

### Step 2: benzyl (1,1,1-trifluoro-3-((2-(2-hydroxypropan-2-yl)-2,3-dihydro-1H-inden-2-yl)amino)-3-oxopropan-2-yl)carbamate

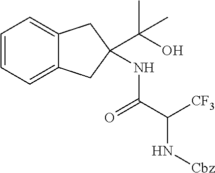 [SMILES: CC(C)(O)C1(NC(=O)C(NC(=O)OCc2ccccc2)C(F)(F)F)Cc2ccccc2C1]

To the mixture of 2-(((benzyloxy)carbonyl)amino)-3,3,3-trifluoropropanoic acid (1.4 g, 5.1 mmol), EDCI (2.5 g, 12.8 mmol) and HOBt (727 mg, 5.4 mmol) in DCM (30 mL) were added 2-(2-amino-2,3-dihydro-1H-inden-2-yl)propan-2-ol (980 mg, 5.1 mmol) and TEA (1.6 g, 15.4 mmol) at 0° C. The mixture was warmed to room temperature and stirred for 1.5 hrs. The reaction mixture was diluted with DCM and quenched by NaHCO₃ (aq.). The organic layer was washed with brine, dried and concentrated in vacuum. The residue was purified by column chromatography on silica gel (eluent: PE/EA (v/v)=1/1). Benzyl (1,1,1-trifluoro-3-((2-(2-hydroxypropan-2-yl)-2,3-dihydro-1H-inden-2-yl)amino)-3-oxopropan-2-yl)carbamate (400 mg, yield: 17%) was obtained. MS (ESI) m/e [M+1]⁺ 451.3.

### Step 3: benzyl (1,1,1-trifluoro-3-((2-(2-hydroxypropan-2-yl)-2,3-dihydro-1H-inden-2-yl)amino)propan-2-yl)carbamate

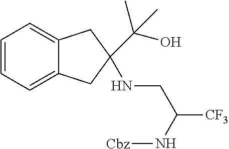 [SMILES: CC(C)(O)C1(NCC(NC(=O)OCc2ccccc2)C(F)(F)F)Cc2ccccc2C1]

Benzyl (1,1,1-trifluoro-3-((2-(2-hydroxypropan-2-yl)-2,3-dihydro-1H-inden-2-yl)amino)-3-oxopropan-2-yl)carbamate (400 mg, 0.89 mmol) was added into BH₃-THF (1M, 10 mL) under stirring. The mixture was heated to 60° C. and stirred for 6 hrs. The reaction mixture was cooled to room temperature and quenched by MeOH (5 mL) at 0° C. The mixture was concentrated in vacuum. The residue was purified by column chromatography on silica gel (eluent: DCM/MeOH (v/v)=25/1). Benzyl (1,1,1-trifluoro-3-((2-(2-hydroxypropan-2-yl)-2,3-dihydro-1H-inden-2-yl)amino)propan-2-yl)carbamate (110 mg, yield: 28%) was obtained. MS (ESI) m/e [M+1]⁺ 437.3.

### Step 4: benzyl (3-((2-(2-((tert-butyldimethylsilyl)oxy)propan-2-yl)-2,3-dihydro-1H-inden-2-yl)amino)-1,1,1-trifluoropropan-2-yl)carbamate

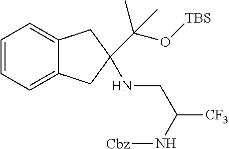

To a solution of benzyl (1,1,1-trifluoro-3-((2-(2-hydroxypropan-2-yl)-2,3-dihydro-1H-inden-2-yl)amino)propan-2-yl)carbamate (110 mg, 0.25 mmol) in DCM (5 mL) were added 2,6-lutidine (40.5 mg, 0.38 mmol) and TBS-OTf (86.6 mg, 0.33 mmol) dropwise at 0° C. under N₂ atmosphere. The mixture was stirred at 0° C. for 1 hour. The reaction mixture was quenched by aq. NaHCO₃ (10 mL) at 0° C. and was extracted with EtOAc (10 mL×3). The combined organic layers were washed with brine, dried and concentrated in vacuum. The residue was purified by column chromatography on silica gel (eluent: PE/EA (v/v)=3/1). Benzyl (3-((2-(2-((tert-butyldimethylsilyl)oxy)propan-2-yl)-2,3-dihydro-1H-inden-2-yl)amino)-1,1,1-trifluoropropan-2-yl)carbamate (110 mg, yield: 79%) was obtained. MS (ESI) m/e [M+1]⁺ 551.4.

### Step 5: N¹-(2-(2-((tert-butyldimethylsilyl)oxy)propan-2-yl)-2,3-dihydro-1H-inden-2-yl)-3,3,3-trifluoropropane-1,2-diamine

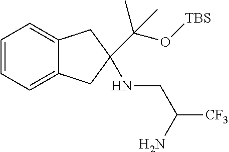

To the solution of benzyl (3-((2-(2-((tert-butyldimethylsilyl)oxy)propan-2-yl)-2,3-dihydro-1H-inden-2-yl)amino)-1,1,1-trifluoropropan-2-yl)carbamate (110 mg, 0.2 mmol) in MeOH (5 mL) was added Pd/C (55 mg). This mixture was then degassed with H₂ and stirred at room temperature for 1 hour at H₂ atmosphere. The reaction mixture was diluted with DCM (5 mL) and then filtered. The filtrate was concentrated in vacuum. N¹-(2-(2-((tert-butyldimethylsilyl)oxy)propan-2-yl)-2,3-dihydro-1H-inden-2-yl)-3,3,3-trifluoropropane-1,2-diamine (84 mg, crude) was obtained, which was used directly in next step. MS (ESI) m/e [M+1]⁺ 417.4.

### Step 6: 1-(2-(2-((tert-butyldimethylsilyl)oxy)propan-2-yl)-2,3-dihydro-1H-inden-2-yl)-4-(trifluoromethyl)imidazolidin-2-one

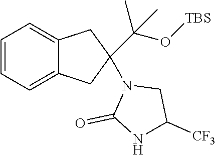

To a solution of N¹-(2-(2-((tert-butyldimethylsilyl)oxy)propan-2-yl)-2,3-dihydro-1H-inden-2-yl)-3,3,3-trifluoropropane-1,2-diamine (84 mg, 0.2 mmol) in THF (6 mL) was added CDI (82 mg, 0.5 mmol) and TEA (102 mg, 1.0 mmol) at room temperature. The mixture was heated to 60° C. and stirred for 2 hrs. The reaction mixture was then cooled to room temperature, diluted with EtOAc (20 mL) and washed with brine. The organic layer was dried and concentrated in vacuum. The residue was purified by Prep-TLC (eluent: EtOAc/DCM (v/v)=2/1). 1-(2-(2-((tert-butyldimethylsilyl)oxy)propan-2-yl)-2,3-dihydro-1H-inden-2-yl)-4-(trifluoromethyl)imidazolidin-2-one (80 mg, crude) was obtained. MS (ESI) m/e [M+1]+443.3.

### Step 7: 1-(2-(2-hydroxypropan-2-yl)-5-nitro-2,3-dihydro-1H-inden-2-yl)-4-(trifluoromethyl)imidazolidin-2-one

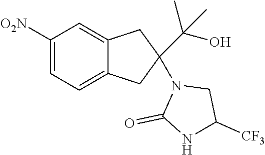 [SMILES: CC(C)(O)C1(N2CC(C(F)(F)F)NC2=O)Cc2ccc([N+](=O)[O-])cc2C1]

To the mixture of H₂SO₄ (2 mL) and HNO₃ (3 mL) was added a solution of (1-(2-(2-((tert-butyldimethylsilyl)oxy)propan-2-yl)-2,3-dihydro-1H-inden-2-yl)-4-(trifluoromethyl)imidazolidin-2-one (80 mg) in AcOH (2 mL) in batches at −5° C. The mixture was further stirred for 5 min at −5° C. The reaction mixture was poured in water/ice and was then extracted with EtOAc (15 mL×3). The combined organic layers were washed with water, aq. NaHCO₃ and brine, dried and concentrated in vacuum. 1-(2-(2-hydroxypropan-2-yl)-5-nitro-2,3-dihydro-1H-inden-2-yl)-4-(trifluoromethyl)imidazolidin-2-one (50 mg, crude) was obtained. MS (ESI) m/e [M+23]⁺ 374.2.

### Step 8: 1-(5-amino-2-(2-hydroxypropan-2-yl)-2,3-dihydro-1H-inden-2-yl)-4-(trifluoromethyl)imidazolidin-2-one

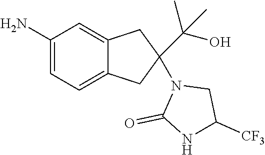 [SMILES: CC(C)(O)C1(N2CC(C(F)(F)F)NC2=O)Cc2ccc(N)cc2C1]

To a solution of 1-(2-(2-hydroxypropan-2-yl)-5-nitro-2,3-dihydro-1H-inden-2-yl)-4-(trifluoromethyl)imidazolidin-2-one (50 mg, crude) in MeOH (6 mL) was added Pd/C (30 mg). The mixture was then degassed with H₂ and stirred at room temperature for 1 hour. The reaction mixture was diluted with DCM (15 mL) and filtered. The filtrate was concentrated in vacuum. 1-(5-amino-2-(2-hydroxypropan-2-yl)-2,3-dihydro-1H-inden-2-yl)-4-(trifluoromethyl)imidazolidin-2-one (40 mg, crude) was obtained. MS (ESI) m/e [M+1]⁺ 344.2.

### Step 9: benzyl ((2S)-1,1-dicyclopropyl-3-((2-(2-hydroxypropan-2-yl)-2-(2-oxo-4-(trifluoromethyl)imidazolidin-1-yl)-2,3-dihydro-1H-inden-5-yl)amino)-3-oxopropan-2-yl)carbamate

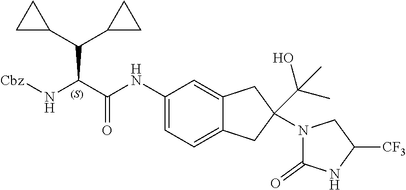

To a solution of 1-(5-amino-2-(2-hydroxypropan-2-yl)-2,3-dihydro-1H-inden-2-yl)-4-(trifluoromethyl)imidazolidin-2-one (40 mg, 0.11 mmol) in DCM (5 mL) was added (S)-2-(((benzyloxy)carbonyl)amino)-3,3-dicyclopropylpropanoic acid (35 mg, 0.11 mmol), DIPEA (45 mg, 0.35 mmol) and HATU (66 mg, 0.17 mmol). The mixture was stirred at room temperature for 1 hour. The reaction mixture was diluted with DCM (15 mL) and was washed with aq. NaHCO₃. The organic layer was washed with brine, dried and concentrated in vacuum. The residue was purified by Prep-TLC (eluent: EtOAc/DCM (v/v)=1/1). Benzyl ((2S)-1,1-dicyclopropyl-3-((2-(2-hydroxypropan-2-yl)-2-(2-oxo-4-(trifluoromethyl)imidazolidin-1-yl)-2,3-dihydro-1H-inden-5-yl)amino)-3-oxopropan-2-yl)carbamate (30 mg, 3 step yield: 41%) was obtained. MS (ESI) m/e [M+1]⁺ 629.1.

### Step 10: (2S)-2-amino-3,3-dicyclopropyl-N-(2-(2-hydroxypropan-2-yl)-2-(2-oxo-4-(trifluoromethyl)imidazolidin-1-yl)-2,3-dihydro-1H-inden-5-yl)propanamide

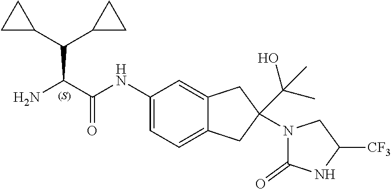

To a solution of benzyl ((2S)-1,1-dicyclopropyl-3-((2-(2-hydroxypropan-2-yl)-2-(2-oxo-4-(trifluoromethyl)imidazolidin-1-yl)-2,3-dihydro-1H-inden-5-yl)amino)-3-oxopropan-2-yl)carbamate (30 mg, 0.05 mmol) in MeOH (6 mL) was added Pd/C (30 mg). The mixture was then degassed with H₂ and stirred for 1 hour under H₂ atmosphere. The reaction mixture was diluted with DCM (10 mL) and filtered. The filtrate was concentrated in vacuum. (2S)-2-amino-3,3-dicyclopropyl-N-(2-(2-hydroxypropan-2-yl)-2-(2-oxo-4-(trifluoromethyl)imidazolidin-1-yl)-2,3-dihydro-1H-inden-5-yl)propanamide (28 mg, crude) was obtained. MS (ESI) m/e [M+1]⁺ 495.2.

### Step 11: N-((2S)-1,1-dicyclopropyl-3-((2-(2-hydroxypropan-2-yl)-2-(2-oxo-4-(trifluoromethyl)imidazolidin-1-yl)-2,3-dihydro-1H-inden-5-yl)amino)-3-oxopropan-2-yl)-4-methyl-1,2,5-oxadiazole-3-carboxamide (Example 279)

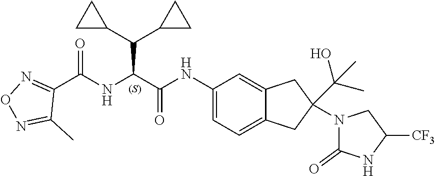

To a solution of (2S)-2-amino-3,3-dicyclopropyl-N-(2-(2-hydroxypropan-2-yl)-2-(2-oxo-4-(trifluoromethyl)imidazolidin-1-yl)-2,3-dihydro-1H-inden-5-yl)propanamide (28 mg, crude) in DCM (5 mL) was added 4-methyl-1,2,5-oxadiazole-3-carboxylic acid (7.3 mg, 0.06 mmol), DIPEA (22 mg, 0.17 mmol) and HATU (33 mg, 0.09 mmol). The mixture was stirred for 1 hour at room temperature. The reaction mixture was diluted with DCM (15 mL) and then washed with aq. NaHCO₃. The organic layer was washed with brine, dried and concentrated in vacuum. The residue was firstly purified by prep-TLC. The crude product was further purified by Prep-HPLC (mobile phase: MeCN/water (0.1% FA); column: SunFire; elution: 50%-68% MeCN). The title compound example 279 (10 mg, yield: 29% for two steps) was obtained. ¹H NMR (400 MHz, DMSO-d₆) δ ppm: 10.06 (s, 1H), 9.01 (d, J=8.8 Hz, 1H), 7.46 (t, J=9.4 Hz, 1H), 7.34-7.30 (m, 2H), 7.12 (d, J=7.8 Hz, 1H), 5.21 (s, 1H), 4.85 (t, J=7.8 Hz, 1H), 4.23 (s, 1H), 3.83-3.57 (m, 3H), 3.43-3.18 (m, 3H), 2.48 (s, 3H), 1.10 (s, 6H), 0.93-0.62 (m, 3H), 0.52-0.11 (m, 8H). MS (ESI) m/e [M+1]⁺ 605.2.

Other examples were synthesized according to the similar methods with Example 32 or Example 170, Example 212, Example 238, Example 256, Example 261, Example 279 which are known to those skilled in this art. The corresponding compound name, structure and spectrum data were listed in the Table.

| Ex. | structure | Compound name |
|---|---|---|
| 1 | 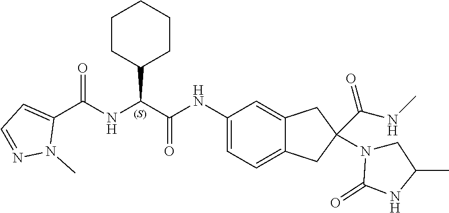 | N-((1S)-1-cyclohexyl- 2-((2-(4-methyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 2 | 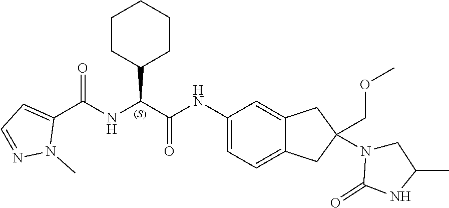 | N-((1S)-1-cyclohexyl- 2-((2-(methoxymethyl)- 2-(4-methyl-2- oxoimidazolidin-1-yl)- 2,3-dihydro-1H-inden- 5-yl)amino)-2- oxoethyl)-1-methyl-1H- pyrazole-5-carboxamide |
| 3 | 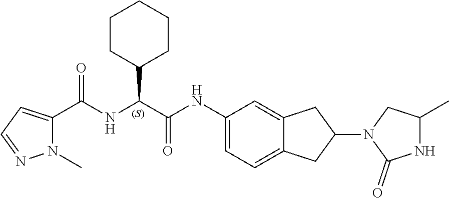 | N-((1S)-1-cyclohexyl- 2-((2-(4-methyl-2- oxoimidazolidin-1-yl)- 2,3-dihydro-1H-inden- 5-yl)amino)-2- oxoethyl)-1-methyl-1H- pyrazole-5-carboxamide |
| 4 | 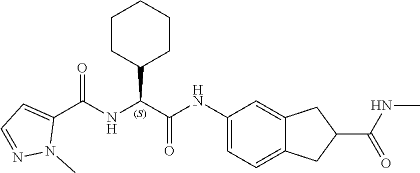 | N-((1S)-1-cyclohexyl- 2-((2- (methylcarbamoyl)-2,3- dihydro-1H-inden-5- yl)amino)-2-oxoethyl)- 1-methyl-1H-pyrazole- 5-carboxamide |
| 5 | 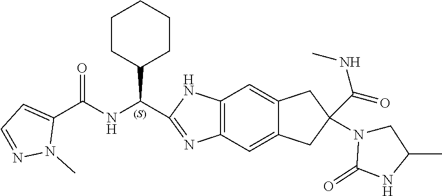 | 2-((S)-cyclohexyl(1- methyl-1H-pyrazole-5- carboxamido)methyl)- N-methyl-6-(4-methyl- 2-oxoimidazolidin-1- yl)-1,5,6,7- tetrahydroindeno[5,6- d]imidazole-6- carboxamide |
| 6 | 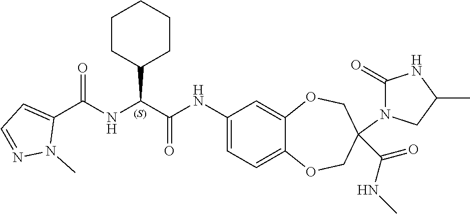 | N-((1S)-1-cyclohexyl- 2-((3-(4-methyl-2- oxoimidazolidin-1-yl)- 3-(methylcarbamoyl)- 3,4-dihydro-2H- benzo[b][1,4]dioxepin- 7-yl)amino)-2- oxoethyl)-1-methyl-1H- pyrazole-5-carboxamide |
| 7 | 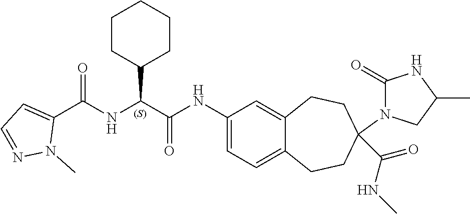 | N-((1S)-1-cyclohexyl- 2-((7-(4-methyl-2- oxoimidazolidin-1-yl)- 7-(methylcarbamoyl)- 6,7,8,9-tetrahydro-5H- benzo[7]annulen-2- yl)amino)-2-oxoethyl)- 1-methyl-1H-pyrazole- 5-carboxamide |
| 8 | 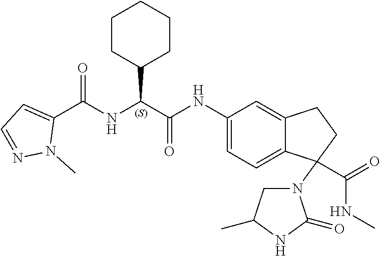 | N-((1S)-1-cyclohexyl- 2-((1-(4-methyl-2- oxoimidazolidin-1-yl)- 1-(methylcarbamoyl)- 2,3-dihydro-1H-inden- 5-yl)amino)-2- oxoethyl)-1-methyl-1H- pyrazole-5-carboxamide |
| 9 | 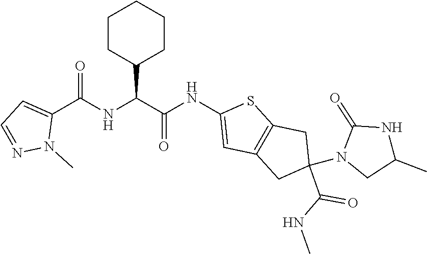 [SMILES: CNC(=O)C1(N2CC(C)NC2=O)Cc2cc(NC(=O)[C@@H](NC(=O)c3ccnn3C)C3CCCCC3)sc2C1] | N-((1S)-1-cyclohexyl- 2-((5-(4-methyl-2- oxoimidazolidin-1-yl)- 5-(methylcarbamoyl)- 5,6-dihydro-4H- cyclopenta[b]thiophen- 2-yl)amino)-2- oxoethyl)-1-methyl-1H- pyrazole-5-carboxamide |
| 10 | 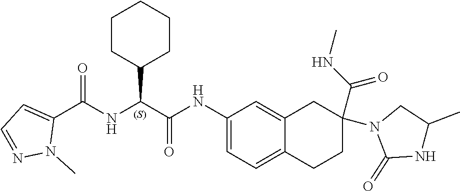 | N-((1S)-1-cyclohexyl- 2-((7-(4-methyl-2- oxoimidazolidin-1-yl)- 7-(methylcarbamoyl)- 5,6,7,8- tetrahydronaphthalen-2- yl)amino)-2-oxoethyl)- 1-methyl-1H-pyrazole- 5-carboxamide |
| 11 | 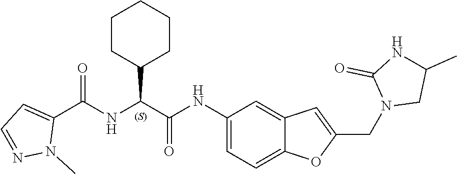 | N-((1S)-1-cyclohexyl- 2-((2-((4-methyl-2- oxoimidazolidin-1- yl)methyl)benzofuran- 5-yl)amino)-2- oxoethyl)-1-methyl-1H- pyrazole-5-carboxamide |
| 12 | 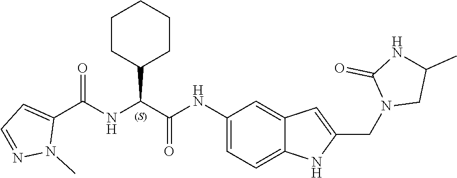 | N-((1S)-1-cyclohexyl- 2-((2-((4-methyl-2- oxoimidazolidin-1- yl)methyl)-1H-indol-5- yl)amino)-2-oxoethyl)- 1-methyl-1H-pyrazole- 5-carboxamide |
| 13 | 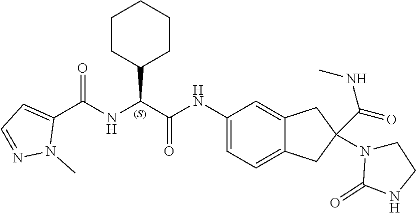 | N-((1S)-1-cyclohexyl- 2-((2- (methylcarbamoyl)-2- (2-oxoimidazolidin-1- yl)-2,3-dihydro-1H- inden-5-yl)amino)-2- oxoethyl)-1-methyl-1H- pyrazole-5-carboxamide |
| 14 | 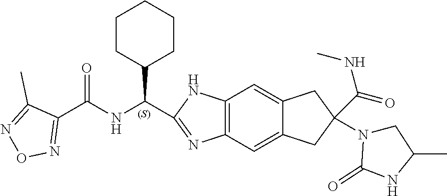 | N-((1S)-cyclohexyl(6- (4-methyl-2- oxoimidazolidin-1-yl)- 6-(methylcarbamoyl)- 1,5,6,7- tetrahydroindeno[5,6- d]imidazol-2- yl)methyl)-4-methyl- 1,2,5-oxadiazole-3- carboxamide |
| 15 | 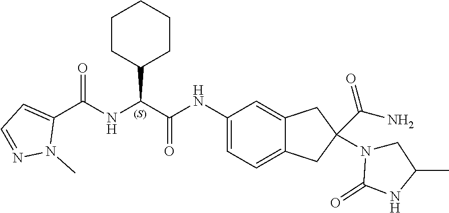 | N-((1S)-2-((2- carbamoyl-2-(4-methyl- 2-oxoimidazolidin-1- yl)-2,3-dihydro-1H- inden-5-yl)amino)-1- cyclohexyl-2-oxoethyl)- 1-methyl-1H-pyrazole- 5-carboxamide |
| 16 | 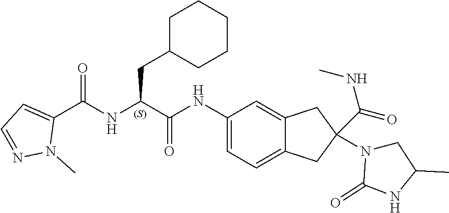 | N-((2S)-3-cyclohexyl- 1-((2-(4-methyl-2- oxoimidazolidin-1-yl)- 2-(methylcarbamoyl)- 2,3-dihydro-1H-inden- 5-yl)amino)-1- oxopropan-2-yl)-1- methyl-1H-pyrazole-5- carboxamide |
| 17 | 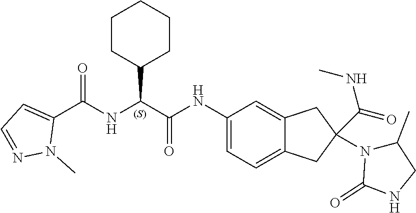 | N-((1S)-1-cyclohexyl- 2-((2-(5-methyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 18 | 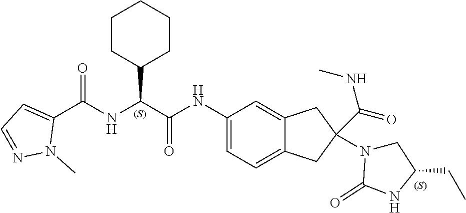 | N-((1S)-1-cyclohexyl- 2-((2-((S)-4-ethyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 19 | 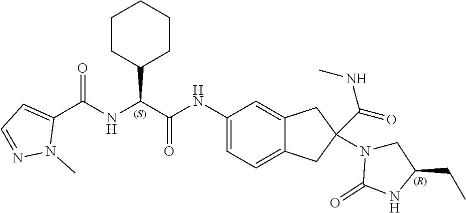 | N-((1S)-1-cyclohexyl- 2-((2-((R)-4-ethyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 20 | 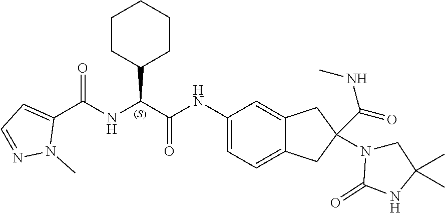 | N-((1S)-1-cyclohexyl- 2-((2-(4,4-dimethyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 21 | 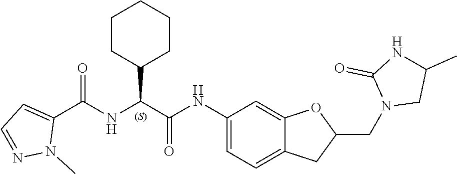 | N-((1S)-1-cyclohexyl- 2-((2-((4-methyl-2- oxoimidazolidin-1- yl)methyl)-2,3- dihydrobenzofuran-6- yl)amino)-2-oxoethyl)- 1-methyl-1H-pyrazole- 5-carboxamide |
| 22 | 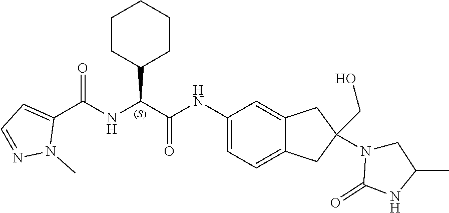 | N-((1S)-1-cyclohexyl- 2-((2-(hydroxymethyl)- 2-(4-methyl-2- oxoimidazolidin-1-yl)- 2,3-dihydro-1H-inden- 5-yl)amino)-2- oxoethyl)-1-methyl-1H- pyrazole-5-carboxamide |
| 23 | 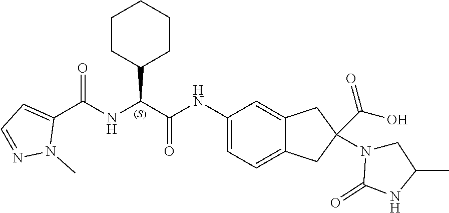 | 5-((S)-2-cyclohexyl-2- (1-methyl-1H-pyrazole- 5- carboxamido) acetamido)- 2-(4-methyl-2- oxoimidazolidin-1-yl)- 2,3-dihydro-1H-indene- 2-carboxylic acid |
| 24 | 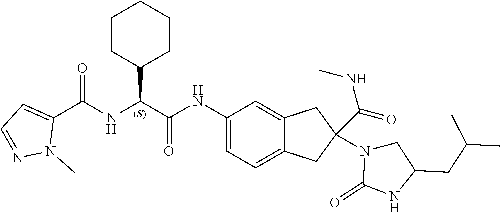 | N-((1S)-1-cyclohexyl- 2-((2-(4-isobutyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 25 | 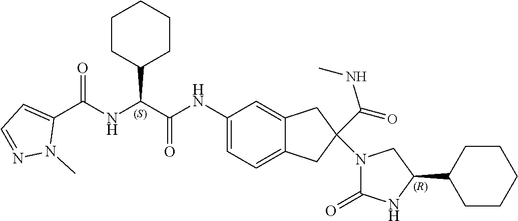 | N-((1S)-1-cyclohexyl- 2-((2-((R)-4- cyclohexyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 26 | 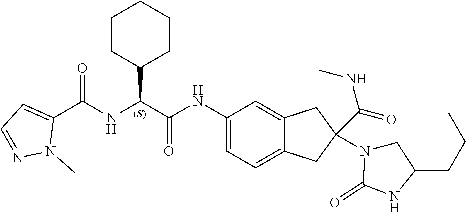 | N-((1S)-1-cyclohexyl- 2-((2- (methylcarbamoyl)-2- (2-oxo-4- propylimidazolidin-1- yl)-2,3-dihydro-1H- inden-5-yl)amino)-2- oxoethyl)-1-methyl-1H- pyrazole-5-carboxamide |
| 27 | 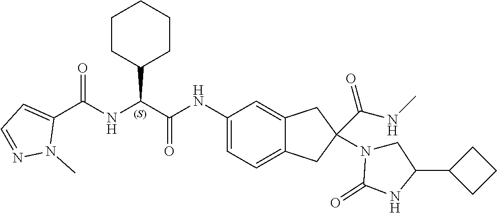 | N-((1S)-2-((2-(4- cyclobutyl-2- oxoimidazolidin-1-yl)- 2-(methylcarbamoyl)- 2,3-dihydro-1H-inden- 5-yl)amino)-1- cyclohexyl-2-oxoethyl)- 1-methyl-1H-pyrazole- 5-carboxamide |
| 28 | 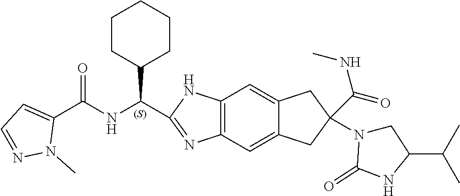 | 2-((S)-cyclohexyl(1- methyl-1H-pyrazole-5- carboxamido)methyl)- 6-(4-isopropyl-2- oxoimidazolidin-1-yl)- N-methyl-1,5,6,7- tetrahydroindeno[5,6- d]imidazole-6- carboxamide |
| 29 | 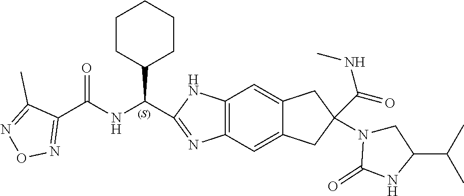 | N-((1S)-cyclohexyl(6- (4-isopropyl-2- oxoimidazolidin-1-yl)- 6-(methylcarbamoyl)- 1,5,6,7- tetrahydroindeno[5,6- d]imidazol-2- yl)methyl)-4-methyl- 1,2,5-oxadiazole-3- carboxamide |
| 30 | 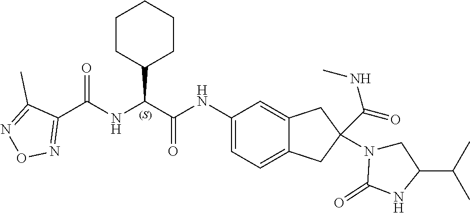 | N-((1S)-1-cyclohexyl- 2-((2-(4-isopropyl-2- oxoimidazolidin-1-yl)- 2-(methylcarbamoyl)- 2,3-dihydro-1H-inden- 5-yl)amino)-2- oxoethyl)-4-methyl- 1,2,5-oxadiazole-3- carboxamide |
| 31 | 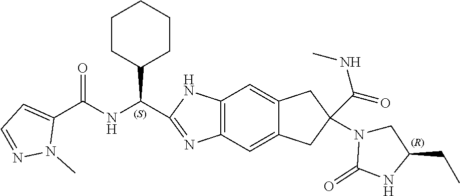 | 2-((S)-cyclohexyl(1- methyl-1H-pyrazole-5- carboxamido)methyl)- 6-((R)-4-ethyl-2- oxoimidazolidin-1-yl)- N-methyl-1,5,6,7- tetrahydroindeno[5,6- d]imidazole-6- carboxamide |
| 33 | 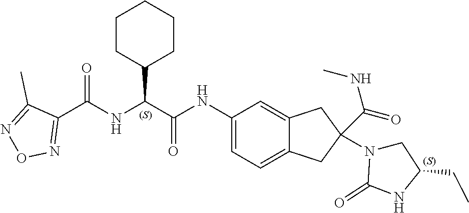 | N-((1S)-cyclohexyl(6- ((S)-4-ethyl-2- oxoimidazolidin-1-yl)- 6-(methylcarbamoyl)- 1,5,6,7- tetrahydroindeno[5,6- d]imidazol-2- yl)methyl)-4-methyl- 1,2,5-oxadiazole-3- carboxamide |
| 34 | 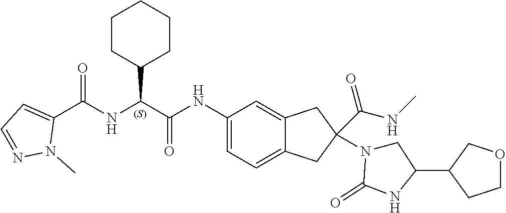 | N-((1S)-1-cyclohexyl- 2-((2- (methylcarbamoyl)-2- (2-oxo-4- (tetrahydrofuran-3- yl)imidazolidin-1-yl)- 2,3-dihydro-1H-inden- 5-yl)amino)-2- oxoethyl)-1-methyl-1H- pyrazole-5-carboxamide |
| 35 | 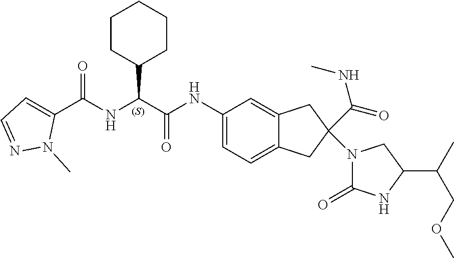 | N-((1S)-1-cyclohexyl- 2-((2-(4-(1- methoxypropan-2-yl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 36 | 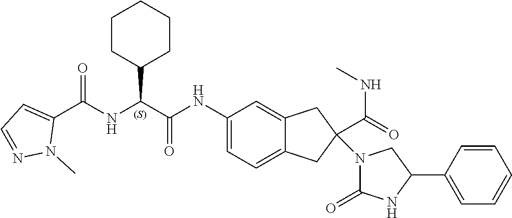 | N-((1S)-1-cyclohexyl- 2-((2- (methylcarbamoyl)-2- (2-oxo-4- phenylimidazolidin-1- yl)-2,3-dihydro-1H- inden-5-yl)amino)-2- oxoethyl)-1-methyl-1H- pyrazole-5-carboxamide |
| 37 | 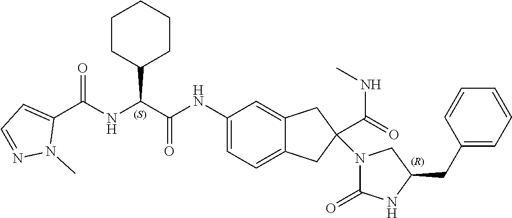 | N-((1S)-2-((2-((R)-4- benzyl-2- oxoimidazolidin-1-yl)- 2-(methylcarbamoyl)- 2,3-dihydro-1H-inden- 5-yl)amino)-1- cyclohexyl-2-oxoethyl)- 1-methyl-1H-pyrazole- 5-carboxamide |
| 38 | 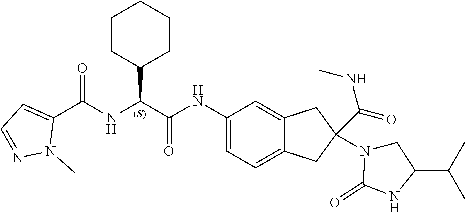 | N-((1S)-1-cyclohexyl- 2-((2-(4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 39 | 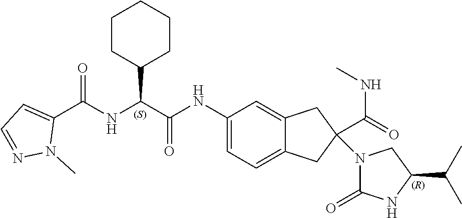 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-methyl-1H-pyrazole- 5-carboxamide |
| 40 | 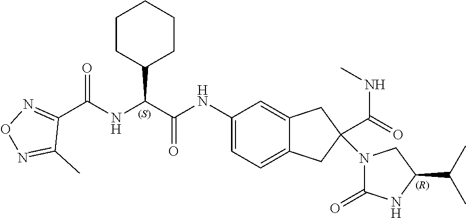 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 4-methyl-1,2,5- oxadiazole-3- carboxamide |
| 41 | 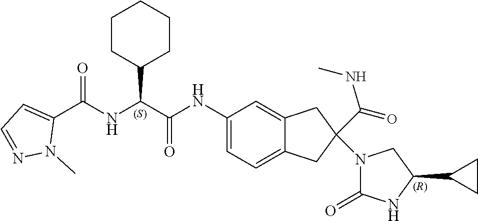 | N-((1S)-1-cyclohexyl- 2-((2-((R)-4- cyclopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 42 | 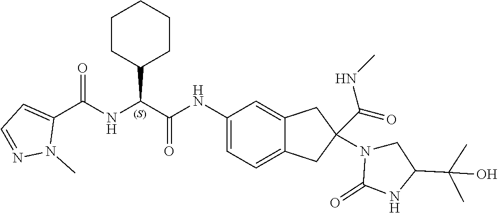 | N-((1S)-1-cyclohexyl- 2-((2-(4-(2- hydroxypropan-2-yl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 43 | 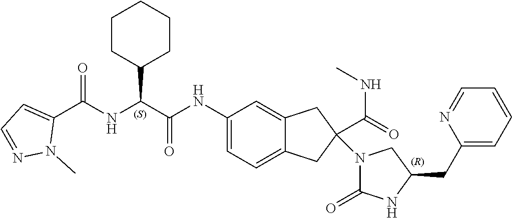 | N-((1S)-1-cyclohexyl- 2-((2- (methylcarbamoyl)-2- ((R)-2-oxo-4-(pyridin- 2- ylmethyl)imidazolidin- 1-yl)-2,3-dihydro-1H- inden-5-yl)amino)-2- oxoethyl)-1-methyl-1H- pyrazole-5-carboxamide |
| 44 | 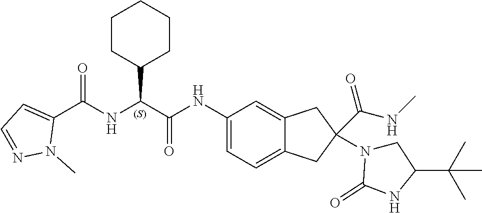 | N-((1S)-2-((2-(4-(tert- butyl)-2- oxoimidazolidin-1-yl)- 2-(methylcarbamoyl)- 2,3-dihydro-1H-inden- 5-yl)amino)-1- cyclohexyl-2-oxoethyl)- 1-methyl-1H-pyrazole- 5-carboxamide |
| 45 | 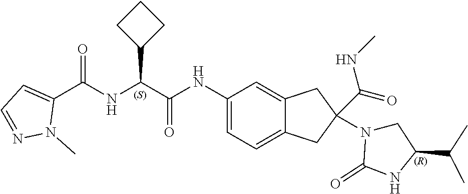 | N-((1S)-1-cyclobutyl-2- ((2-((R)-4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 46 | 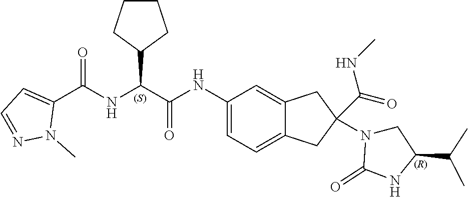 | N-((1S)-1-cyclopentyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-methyl-1H-pyrazole- 5-carboxamide |
| 47 | 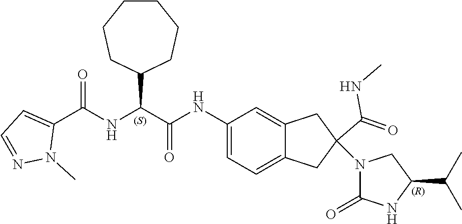 | N-((1S)-1-cycloheptyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-methyl-1H-pyrazole- 5-carboxamide |
| 48 | 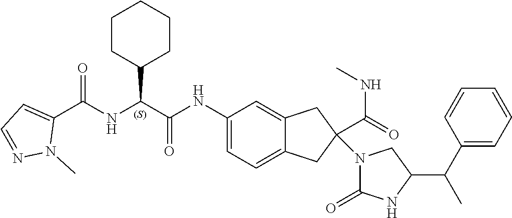 | N-((1S)-1-cyclohexyl- 2-((2- (methylcarbamoyl)-2- (2-oxo-4-(1- phenylethyl)imidazolidin- 1-yl)-2,3-dihydro-1H- inden-5-yl)amino)-2- oxoethyl)-1-methyl-1H- pyrazole-5-carboxamide |
| 49 | 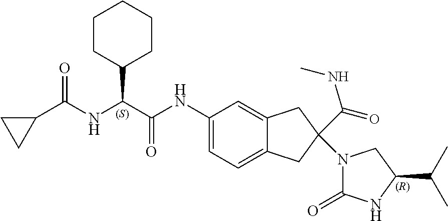 | 5-((S)-2-cyclohexyl-2- (cyclopropanecarboxamido) acetamido)-2-((R)- 4-isopropyl-2- oxoimidazolidin-1-yl)- N-methyl-2,3-dihydro- 1H-indene-2- carboxamide |
| 50 | 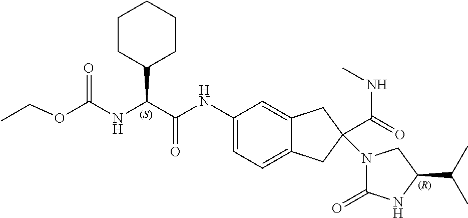 | ethyl ((1S)-1- cyclohexyl-2-((2-((R)- 4-isopropyl-2- oxoimidazolidin-1-yl)- 2-(methylcarbamoyl)- 2,3-dihydro-1H-inden- 5-yl)amino)-2- oxoethyl)carbamate |
| 51 | 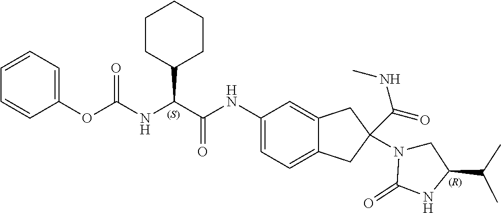 | phenyl ((1S)-1- cyclohexyl-2-((2-((R)- 4-isopropyl-2- oxoimidazolidin-1-yl)- 2-(methylcarbamoyl)- 2,3-dihydro-1H-inden- 5-yl)amino)-2- oxoethyl)carbamate |
| 52 | 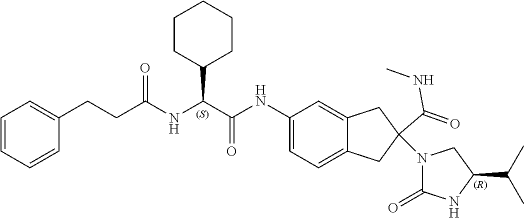 | 5-((S)-2-cyclohexyl-2- (3- phenylpropanamido) acetamido)-2-((R)-4- isopropyl-2- oxoimidazolidin-1-yl)- N-methyl-2,3-dihydro- 1H-indene-2- carboxamide |
| 53 | 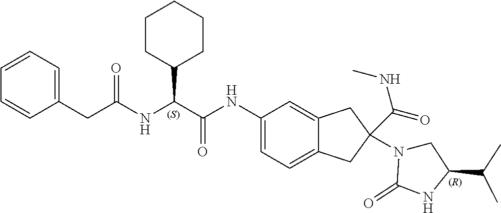 | 5-((S)-2-cyclohexyl-2- (2- phenylacetamido) acetamido)-2-((R)-4- isopropyl-2- oxoimidazolidin-1-yl)- N-methyl-2,3-dihydro- 1H-indene-2- carboxamide |
| 54 | 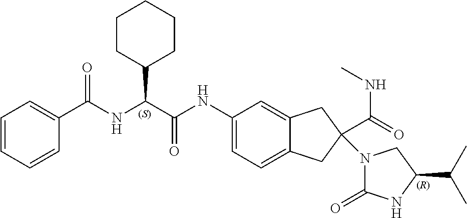 | 5-((S)-2-benzamido-2- cyclohexylacetamido)- 2-((R)-4-isopropyl-2- oxoimidazolidin-1-yl)- N-methyl-2,3-dihydro- 1H-indene-2- carboxamide |
| 55 | 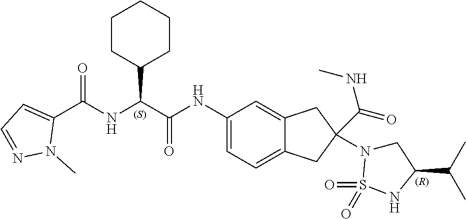 | N-((1S)-1-cyclohexyl- 2-((2-((R)-4-isopropyl- 1,1-dioxido-1,2,5- thiadiazolidin-2-yl)-2- (methylcarbamoyl)-2,3- dihydro-1H-inden-5- yl)amino)-2-oxoethyl)- 1-methyl-1H-pyrazole- 5-carboxamide |
| 56 | 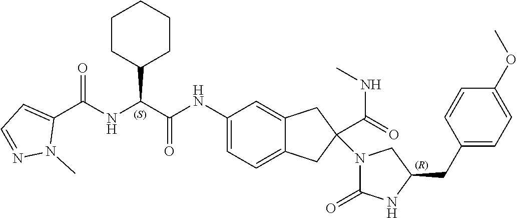 | N-((1S)-1-cyclohexyl- 2-((2-((R)-4-(4- methoxybenzyl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 57 | 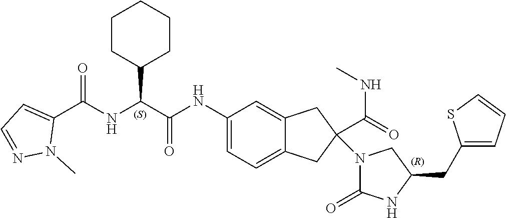 | N-((1S)-1-cyclohexyl- 2-((2- (methylcarbamoyl)-2- ((R)-2-oxo-4-(thiophen- 2- ylmethyl)imidazolidin- 1-yl)-2,3-dihydro-1H- inden-5-yl)amino)-2- oxoethyl)-1-methyl-1H- pyrazole-5-carboxamide |
| 58 | 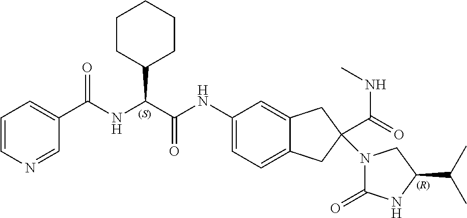 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2- oxoethyl)nicotinamide |
| 59 | 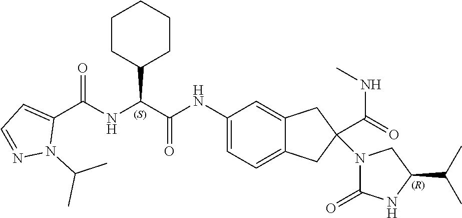 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-isopropyl-1H- pyrazole-5-carboxamide |
| 60 | 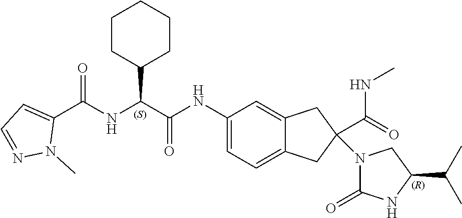 | N-((1S)-2-((2-((R)-4- isopropyl-2- oxoimidazolidin-1-yl)- 2-(methylcarbamoyl)- 2,3-dihydro-1H-inden- 5-yl)amino)-2-oxo-1- phenylethyl)-1-methyl- 1H-pyrazole-5- carboxamide |
| 61 | 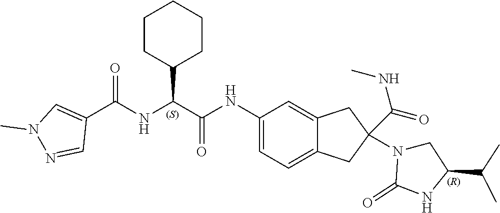 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-methyl-1H-pyrazole- 4-carboxamide |
| 62 | 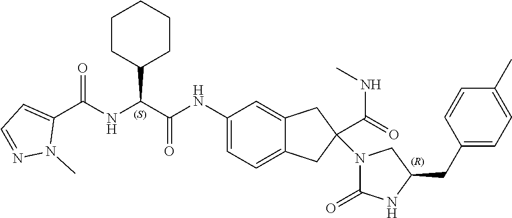 | N-((1S)-1-cyclohexyl- 2-((2-((R)-4-(4- methylbenzyl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 63 | 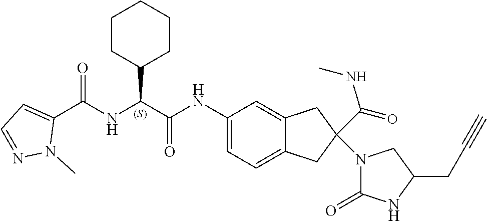 | N-((1S)-1-cyclohexyl- 2-((2- (methylcarbamoyl)-2- (2-oxo-4-(prop-2-yn-1- yl)imidazolidin-1-yl)- 2,3-dihydro-1H-inden- 5-yl)amino)-2- oxoethyl)-1-methyl-1H- pyrazole-5-carboxamide |
| 64 | 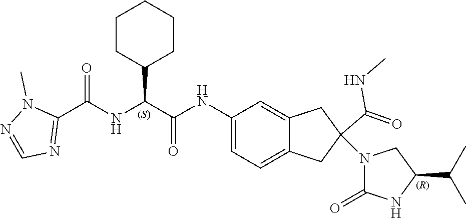 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-methyl-1H-1,2,4- triazole-5-carboxamide |
| 65 | 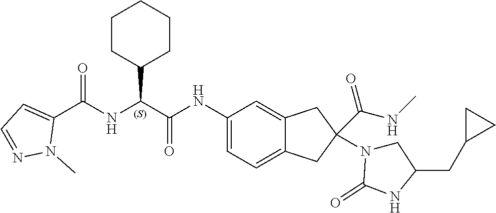 | N-((1S)-1-cyclohexyl- 2-((2-(4- (cyclopropylmethyl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 66 | 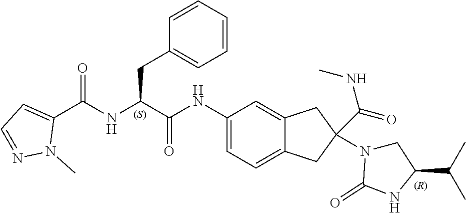 | N-((2S)-1-((2-((R)-4- isopropyl-2- oxoimidazolidin-1-yl)- 2-(methylcarbamoyl)- 2,3-dihydro-1H-inden- 5-yl)amino)-1-oxo-3- phenylpropan-2-yl)-1- methyl-1H-pyrazole-5- carboxamide |
| 67 | 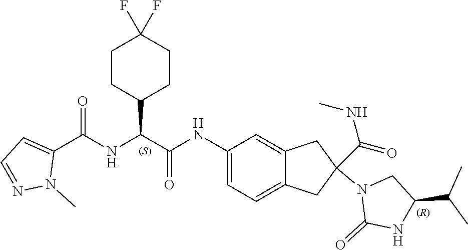 | N-((1S)-1-(4,4- difluorocyclohexyl)-2- ((2-((R)-4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 68 | 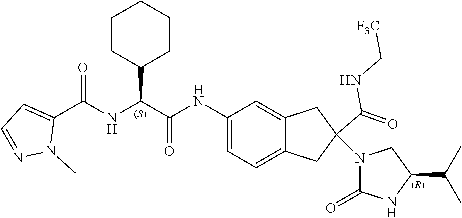 | N-((1S)-1-cyclohexyl- 2-((2-((R)-4-isopropyl- 2-oxoimidazolidin-1- yl)-2-((2,2,2- trifluoroethyl)carbamoyl)- 2,3-dihydro-1H- inden-5-yl)amino)-2- oxoethyl)-1-methyl-1H- pyrazole-5-carboxamide |
| 69 | 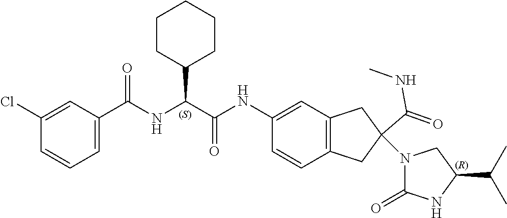 | 5-((S)-2-(3- chlorobenzamido)-2- cyclohexylacetamido)- 2-((R)-4-isopropyl-2- oxoimidazolidin-1-yl)- N-methyl-2,3-dihydro- 1H-indene-2- carboxamide |
| 70 | 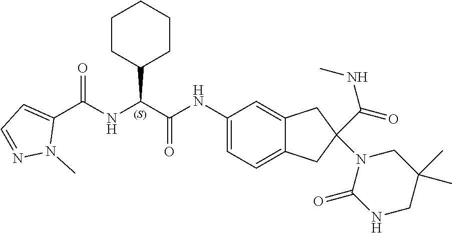 | N-((1S)-1-cyclohexyl- 2-((2-(5,5-dimethyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 71 | 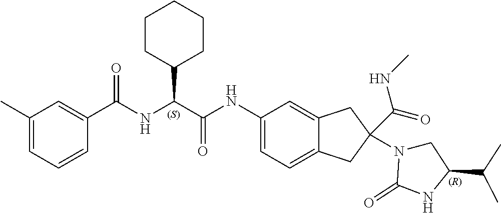 | 5-((S)-2-cyclohexyl-2- (3- methylbenzamido) acetamido)- 2-((R)-4- isopropyl-2- oxoimidazolidin-1-yl)- N-methyl-2,3-dihydro- 1H-indene-2- carboxamide |
| 72 | 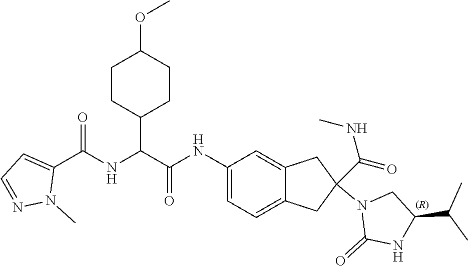 | N-(2-((2-((R)-4- isopropyl-2- oxoimidazolidin-1-yl)- 2-(methylcarbamoyl)- 2,3-dihydro-1H-inden- 5-yl)amino)-1-(4- methoxycyclohexyl)-2- oxoethyl)-1-methyl-1H- pyrazole-5-carboxamide |
| 73 | 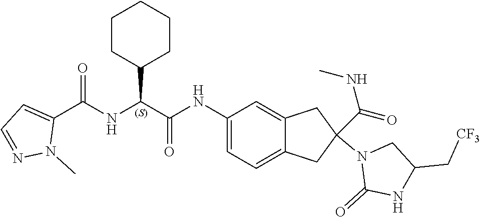 | N-((1S)-1-cyclohexyl- 2-((2- (methylcarbamoyl)-2- (2-oxo-4-(2,2,2- trifluoroethyl) imidazolidin- 1-yl)-2,3-dihydro- 1H-inden-5-yl)amino)- 2-oxoethyl)-1-methyl- 1H-pyrazole-5- carboxamide |
| 74 | 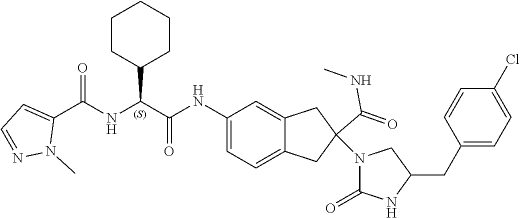 | N-((1S)-2-((2-(4-(4- chlorobenzyl)-2- oxoimidazolidin-1-yl)- 2-(methylcarbamoyl)- 2,3-dihydro-1H-inden- 5-yl)amino)-1- cyclohexyl-2-oxoethyl)- 1-methyl-1H-pyrazole- 5-carboxamide |
| 75 | 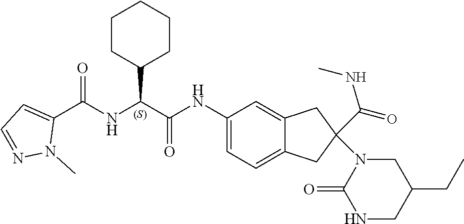 | N-((1S)-1-cyclohexyl- 2-((2-(5-ethyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 76 | 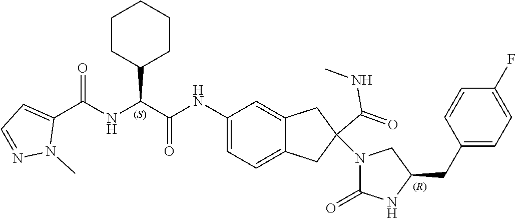 | N-((1S)-1-cyclohexyl- 2-((2-((R)-4-(4- fluorobenzyl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 77 | 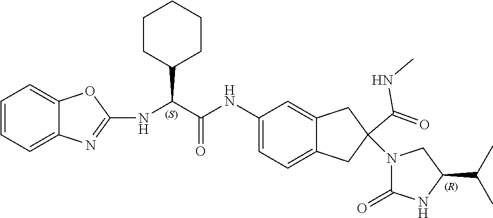 | 5-((S)-2- (benzo[d]oxazol-2- ylamino)-2- cyclohexylacetamido)- 2-((R)-4-isopropyl-2- oxoimidazolidin-1-yl)- N-methyl-2,3-dihydro- 1H-indene-2- carboxamide |
| 78 | 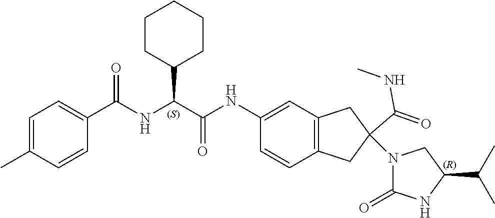 | 5-((S)-2-cyclohexyl-2- (4- methylbenzamido) acetamido)- 2-((R)-4- isopropyl-2- oxoimidazolidin-1-yl)- N-methyl-2,3-dihydro- 1H-indene-2- carboxamide |
| 79 | 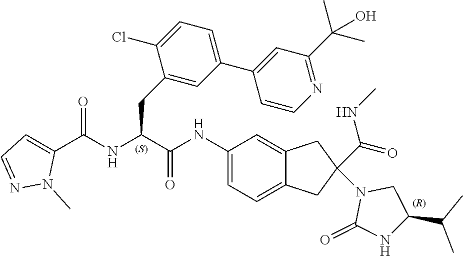 | N-((2S)-3-(2-chloro-5- (2-(2-hydroxypropan-2- yl)pyridin-4-yl)phenyl)- 1-((2-((R)-4-isopropyl- 2-oxoimidazolidin-1- yl)-2- (methylcarbamoyl)-2,3- dihydro-1H-inden-5- yl)amino)-1-oxopropan- 2-yl)-1-methyl-1H- pyrazole-5-carboxamide |
| 80 | 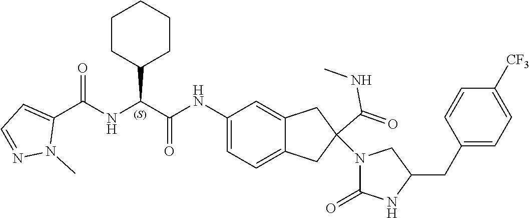 | N-((1S)-1-cyclohexyl- 2-((2- (methylcarbamoyl)-2- (2-oxo-4-(4- (trifluoromethyl)benzyl) imidazolidin-1-yl)-2,3- dihydro-1H-inden-5- yl)amino)-2-oxoethyl)- 1-methyl-1H-pyrazole- 5-carboxamide |
| 81 | 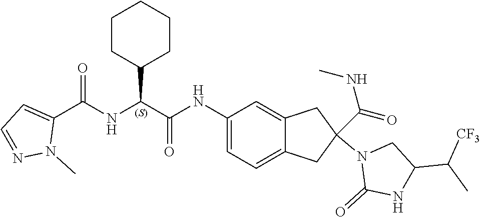 | N-((1S)-1-cyclohexyl- 2-((2- (methylcarbamoyl)-2- (2-oxo-4-(1,1,1- trifluoropropan-2- yl)imidazolidin-1-yl)- 2,3-dihydro-1H-inden- 5-yl)amino)-2- oxoethyl)-1-methyl-1H- pyrazole-5-carboxamide |
| 82 | 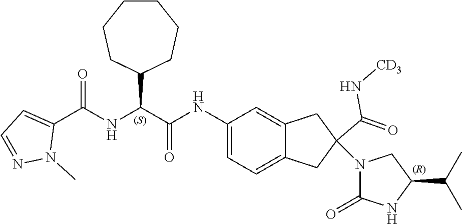 | N-((1S)-1-cycloheptyl- 2-((2-((R)-4-isopropyl- 2-oxoimidazolidin-1- yl)-2-((methyl- d3)carbamoyl)-2,3- dihydro-1H-inden-5- yl)amino)-2-oxoethyl)- 1-methyl-1H-pyrazole- 5-carboxamide |
| 83 | 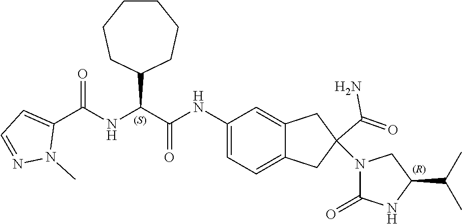 | N-((1S)-2-((2- carbamoyl-2-((R)-4- isopropyl-2- oxoimidazolidin-1-yl)- 2,3-dihydro-1H-inden- 5-yl)amino)-1- cycloheptyl-2- oxoethyl)-1-methyl-1H- pyrazole-5-carboxamide |
| 84 | 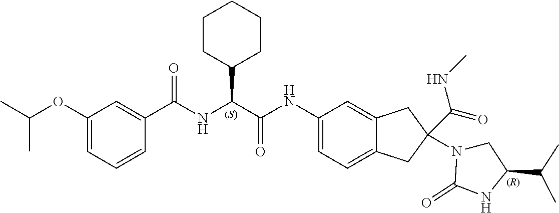 | 5-((S)-2-cyclohexyl-2- (3- isopropoxybenzamido) acetamido)-2-((R)-4- isopropyl-2- oxoimidazolidin-1-yl)- N-methyl-2,3-dihydro- 1H-indene-2- carboxamide |
| 85 | 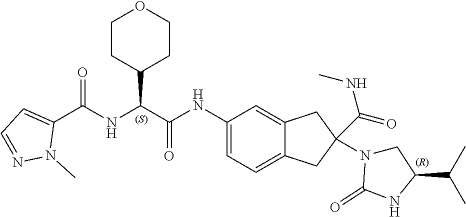 | N-((1S)-2-((2-((R)-4- isopropyl-2- oxoimidazolidin-1-yl)- 2-(methylcarbamoyl)- 2,3-dihydro-1H-inden- 5-yl)amino)-2-oxo-1- (tetrahydro-2H-pyran-4- yl)ethyl)-1-methyl-1H- pyrazole-5-carboxamide |
| 86 | 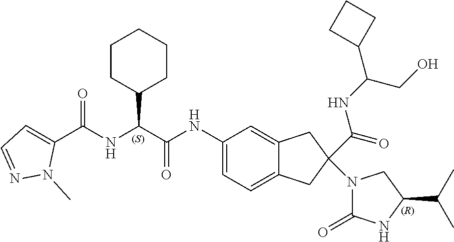 | N-((1S)-2-((2-((1- cyclobutyl-2- hydroxyethyl) carbamoyl)- 2-((R)-4-isopropyl-2- oxoimidazolidin-1-yl)- 2,3-dihydro-1H-inden- 5-yl)amino)-1- cyclohexyl-2-oxoethyl)- 1-methyl-1H-pyrazole- 5-carboxamide |
| 87 | 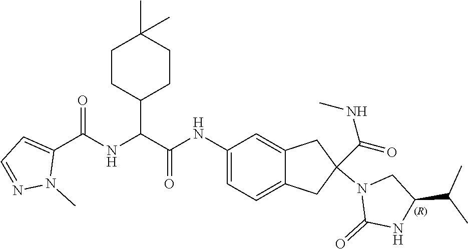 | N-(1-(4,4- dimethylcyclohexyl)-2- ((2-((R)-4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 88 | 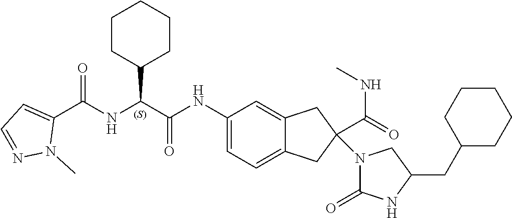 | N-((1S)-1-cyclohexyl- 2-((2-(4- (cyclohexylmethyl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 89 | 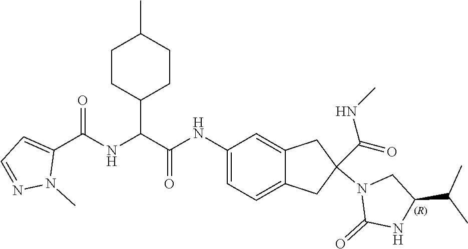 | N-(2-((2-((R)-4- isopropyl-2- oxoimidazolidin-1-yl)- 2-(methylcarbamoyl)- 2,3-dihydro-1H-inden- 5-yl)amino)-1-(4- methylcyclohexyl)-2- oxoethyl)-1-methyl-1H- pyrazole-5-carboxamide |
| 90 | 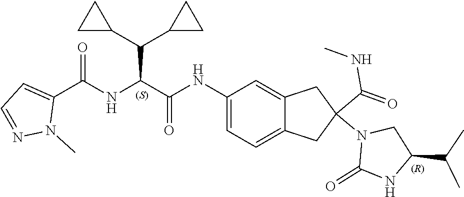 | N-((2S)-1,1- dicyclopropyl-3-((2- ((R)-4-isopropyl-2- oxoimidazolidin-1-yl)- 2-(methylcarbamoyl)- 2,3-dihydro-1H-inden- 5-yl)amino)-3- oxopropan-2-yl)-1- methyl-1H-pyrazole-5- carboxamide |
| 91 | 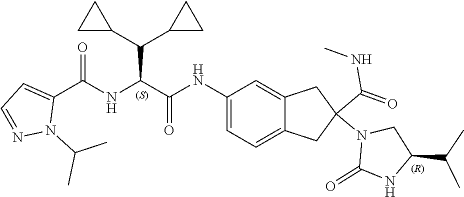 | N-((2S)-1,1- dicyclopropyl-3-((2- ((R)-4-isopropyl-2- oxoimidazolidin-1-yl)- 2-(methylcarbamoyl)- 2,3-dihydro-1H-inden- 5-yl)amino)-3- oxopropan-2-yl)-1- isopropyl-1H-pyrazole- 5-carboxamide |
| 92 | 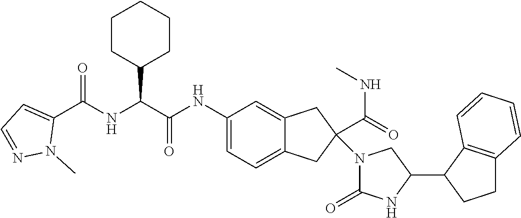 [SMILES: CNC(=O)C1(N2CC(C3CCc4ccccc43)NC2=O)Cc2ccc(NC(=O)[C@@H](NC(=O)c3ccnn3C)C3CCCCC3)cc2C1] | N-((1S)-1-cyclohexyl- 2-((2-(4-(2,3-dihydro- 1H-inden-1-yl)-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 93 | 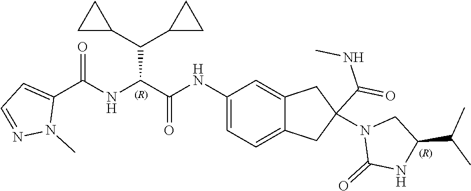 | N-((2R)-1,1- dicyclopropyl-3-((2- ((R)-4-isopropyl-2- oxoimidazolidin-1-yl)- 2-(methylcarbamoyl)- 2,3-dihydro-1H-inden- 5-yl)amino)-3- oxopropan-2-yl)-1- methyl-1H-pyrazole-5- carboxamide |
| 94 | 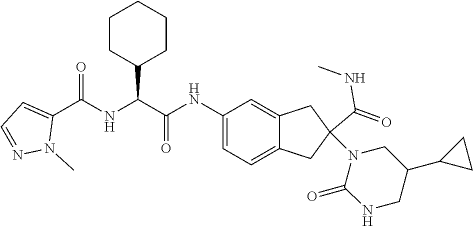 [SMILES: CNC(=O)C1(N2CC(C3CC3)CNC2=O)Cc2ccc(NC(=O)[C@@H](NC(=O)c3ccnn3C)C3CCCCC3)cc2C1] | N-((1S)-1-cyclohexyl- 2-((2-(5-cyclopropyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 95 | 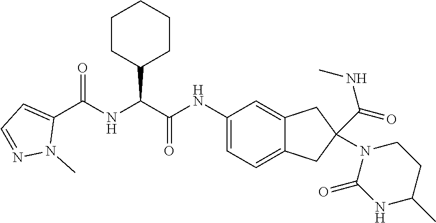 [SMILES: CNC(=O)C1(N2CCC(C)NC2=O)Cc2ccc(NC(=O)[C@@H](NC(=O)c3ccnn3C)C3CCCCC3)cc2C1] | N-((1S)-1-cyclohexyl- 2-((2-(4-methyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 96 | 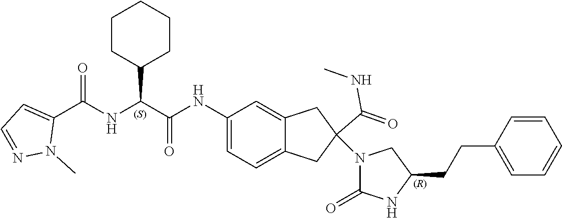 | N-((1S)-1-cyclohexyl- 2-((2- (methylcarbamoyl)-2- ((R)-2-oxo-4- phenethylimidazolidin- 1-yl)-2,3-dihydro-1H- inden-5-yl)amino)-2- oxoethyl)-1-methyl-1H- pyrazole-5-carboxamide |
| 97 | 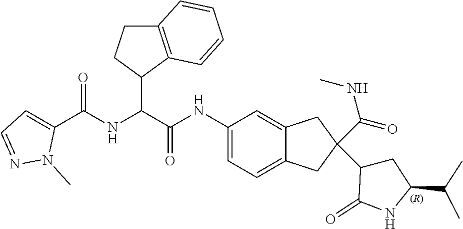 | N-(1-(2,3-dihydro-1H- inden-1-yl)-2-((2-((R)- 4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 98 | 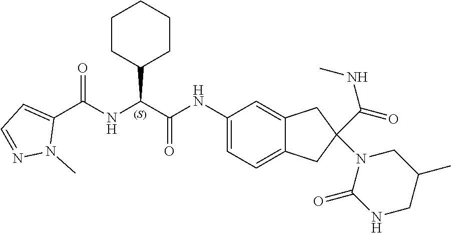 | N-((1S)-1-cyclohexyl- 2-((2-(5-methyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 99 | 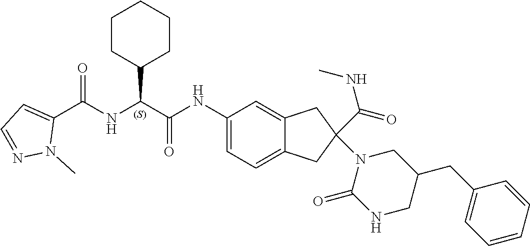 | N-((1S)-2-((2-(5- benzyl-2- oxotetrahydropyrimidin- 1(2H)-yl)-2- (methylcarbamoyl)-2,3- dihydro-1H-inden-5- yl)amino)-1- cyclohexyl-2-oxoethyl)- 1-methyl-1H-pyrazole- 5-carboxamide |
| 100 | 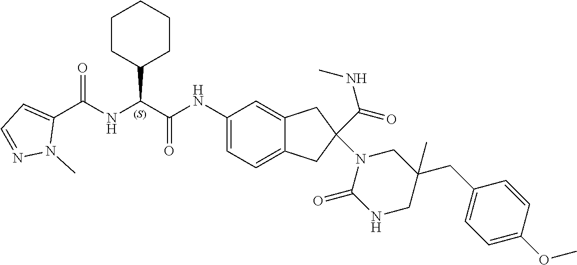 | N-((1S)-1-cyclohexyl- 2-((2-(5-(4- methoxybenzyl)-5- methyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 101 | 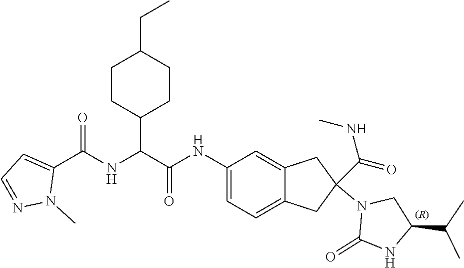 | N-(1-(4- ethylcyclohexyl)-2-((2- ((R)-4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 102 | 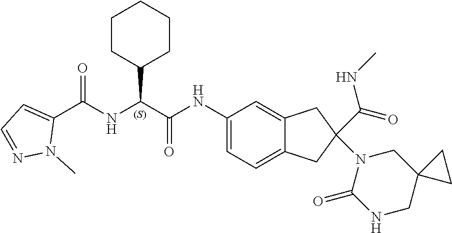 | N-((1S)-1-cyclohexyl- 2-((2- (methylcarbamoyl)-2- (6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 103 | 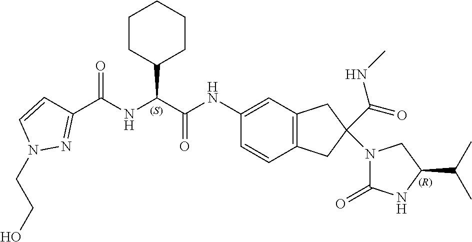 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-(2-hydroxyethyl)-1H- pyrazole-3-carboxamide |
| 104 | 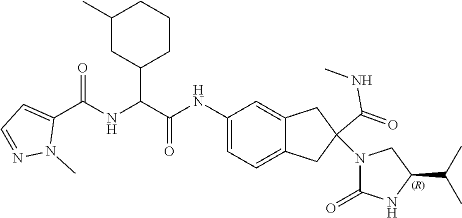 | N-(2-((2-((R)-4- isopropyl-2- oxoimidazolidin-1-yl)- 2-(methylcarbamoyl)- 2,3-dihydro-1H-inden- 5-yl)amino)-1-(3- methylcyclohexyl)-2- oxoethyl)-1-methyl-1H- pyrazole-5-carboxamide |
| 105 | 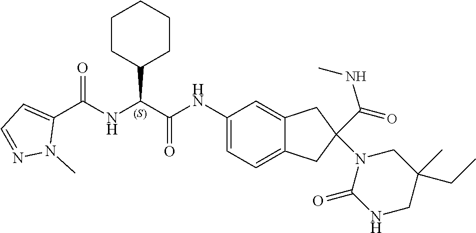 | N-((1S)-1-cyclohexyl- 2-((2-(5-ethyl-5-methyl- 2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 106 | 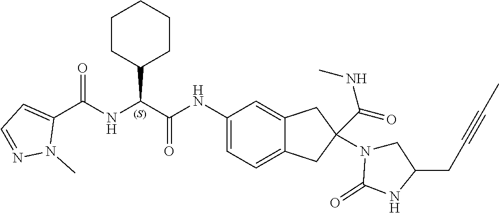 | N-((1S)-2-((2-(4-(but-2- yn-1-yl)-2- oxoimidazolidin-1-yl)- 2-(methylcarbamoyl)- 2,3-dihydro-1H-inden- 5-yl)amino)-1- cyclohexyl-2-oxoethyl)- 1-methyl-1H-pyrazole- 5-carboxamide |
| 107 | 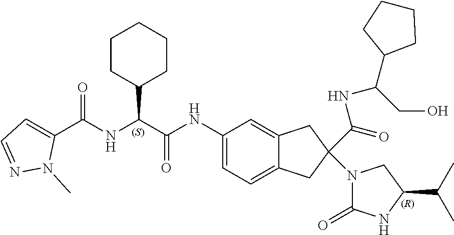 | N-((1S)-1-cyclohexyl- 2-((2-((1-cyclopentyl-2- hydroxyethyl)carbamoyl)- 2-((R)-4-isopropyl-2- oxoimidazolidin-1-yl)- 2,3-dihydro-1H-inden- 5-yl)amino)-2- oxoethyl)-1-methyl-1H- pyrazole-5-carboxamide |
| 108 | 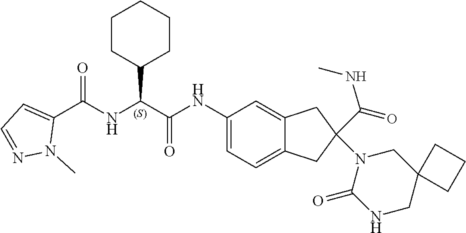 | N-((1S)-1-cyclohexyl- 2-((2- (methylcarbamoyl)-2- (7-oxo-6,8- diazaspiro[3.5]nonan-6- yl)-2,3-dihydro-1H- inden-5-yl)amino)-2- oxoethyl)-1-methyl-1H- pyrazole-5-carboxamide |
| 109 | 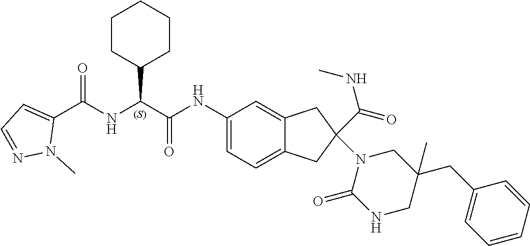 | N-((1S)-2-((2-(5- benzyl-5-methyl-2- oxotetrahydropyrimidin- 1(2H)-yl)-2- (methylcarbamoyl)-2,3- dihydro-1H-inden-5- yl)amino)-1- cyclohexyl-2-oxoethyl)- 1-methyl-1H-pyrazole- 5-carboxamide |
| 110 | 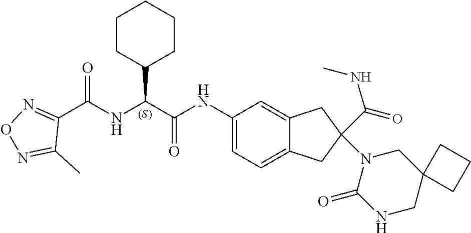 | N-((1S)-1-cyclohexyl- 2-((2- (methylcarbamoyl)-2- (7-oxo-6,8- diazaspiro[3.5]nonan-6- yl)-2,3-dihydro-1H- inden-5-yl)amino)-2- oxoethyl)-4-methyl- 1,2,5-oxadiazole-3- carboxamide |
| 111 | 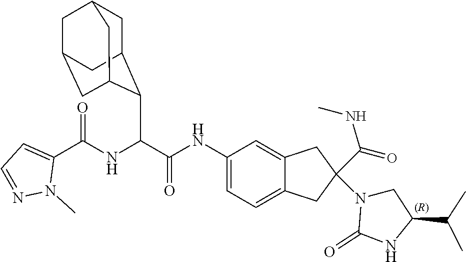 | [0344] N-(1- ((1R,3S,5R,7R)- adamantan-2-yl)-2-((2- ((R)-4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 112 | 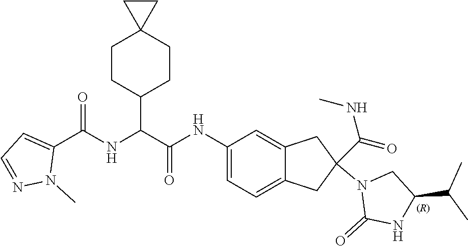 | N-(2-((2-((R)-4- isopropyl-2- oxoimidazolidin-1-yl)- 2-(methylcarbamoyl)- 2,3-dihydro-1H-inden- 5-yl)amino)-2-oxo-1- (spiro[2.5]octan-6- yl)ethyl)-1-methyl-1H- pyrazole-5-carboxamide |
| 113 | 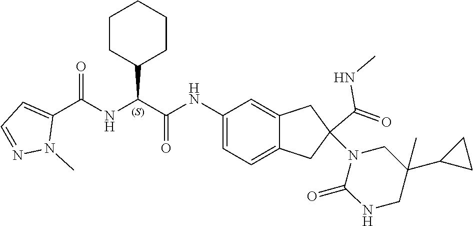 | N-((1S)-1-cyclohexyl- 2-((2-(5-cyclopropyl-5- methyl-2- oxotetrahydropyrimidin- 1(2H)-yl)-2- (methylcarbamoyl)-2,3- dihydro-1H-inden-5- yl)amino)-2-oxoethyl)- 1-methyl-1H-pyrazole- 5-carboxamide |
| 114 | 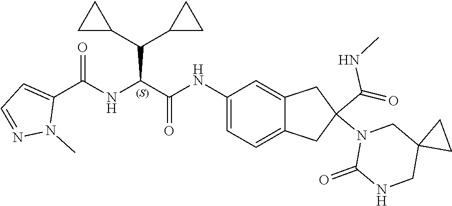 | N-((2S)-1,1- dicyclopropyl-3-((2- (methylcarbamoyl)-2- (6-oxo-5,7- diazaspiro[2.5]octan-5- yl)-2,3-dihydro-1H- inden-5-yl)amino)-3- oxopropan-2-yl)-1- methyl-1H-pyrazole-5- carboxamide |
| 115 | 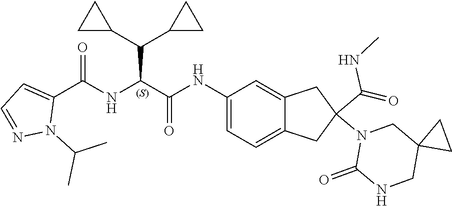 | N-((2S)-1,1- dicyclopropyl-3-((2- (methylcarbamoyl)-2- (6-oxo-5,7- diazaspiro[2.5]octan-5- yl)-2,3-dihydro-1H- inden-5-yl)amino)-3- oxopropan-2-yl)-1- isopropyl-1H-pyrazole- 5-carboxamide |
| 116 | 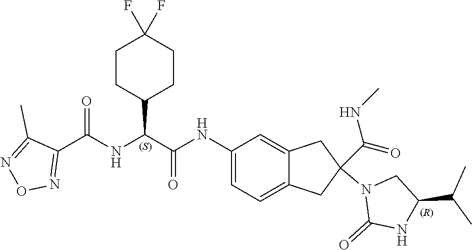 | N-((1S)-1-(4,4- difluorocyclohexyl)-2- ((2-((R)-4-isopropyl-2- oxoimidazolidin-1-yl)- 2-(methylcarbamoyl)- 2,3-dihydro-1H-inden- 5-yl)amino)-2- oxoethyl)-4-methyl- 1,2,5-oxadiazole-3- carboxamide |
| 117 | 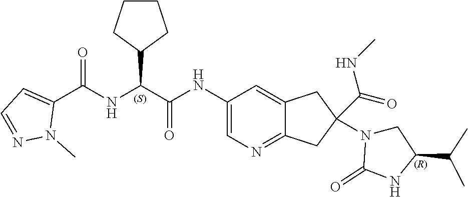 | 3-((S)-2-cyclohexyl-2- (1-methyl-1H-pyrazole- 5- carboxamido)acetamido)- 6-((R)-4-isopropyl-2- oxoimidazolidin-1-yl)- N-methyl-6,7-dihydro- 5H- cyclopenta[b]pyridine- 6-carboxamide |
| 118 | 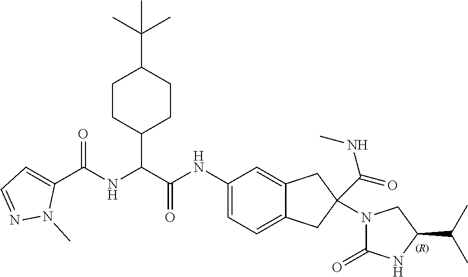 | N-(1-(4-(tert- butyl)cyclohexyl)-2-((2- ((R)-4-isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 119 | 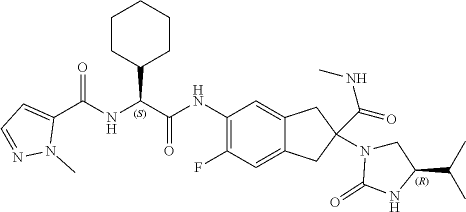 | N-((1S)-1-cyclohexyl- 2-((6-fluoro-2-((R)-4- isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 120 | 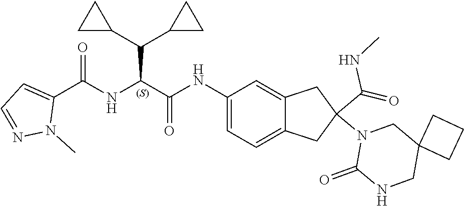 | N-((2S)-1,1- dicyclopropyl-3-((2- (methylcarbamoyl)-2- (7-oxo-6,8- diazaspiro[3.5]nonan-6- yl)-2,3-dihydro-1H- inden-5-yl)amino)-3- oxopropan-2-yl)-1- methyl-1H-pyrazole-5- carboxamide |
| 121 | 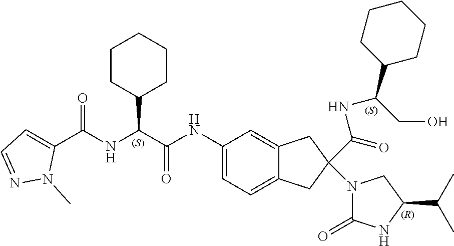 | N-((1S)-1-cyclohexyl- 2-((2-(((S)-1- cyclohexyl-2- hydroxyethyl)carbamoyl)- 2-((R)-4-isopropyl-2- oxoimidazolidin-1-yl)- 2,3-dihydro-1H-inden- 5-yl)amino)-2- oxoethyl)-1-methyl-1H- pyrazole-5-carboxamide |
| 122 | 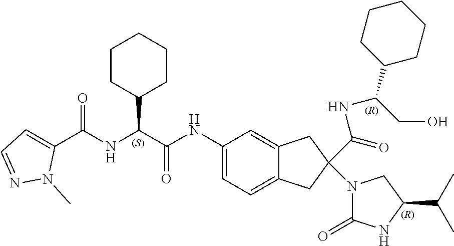 | N-((1S)-1-cyclohexyl- 2-((2-(((R)-1- cyclohexyl-2- hydroxyethyl)carbamoyl)- 2-((R)-4-isopropyl-2- oxoimidazolidin-1-yl)- 2,3-dihydro-1H-inden- 5-yl)amino)-2- oxoethyl)-1-methyl-1H- pyrazole-5-carboxamide |
| 123 | 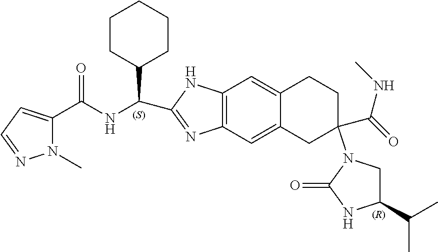 | 2-((S)-cyclohexyl(1- methyl-1H-pyrazole-5- carboxamido)methyl)- 6-((R)-4-isopropyl-2- oxoimidazolidin-1-yl)- N-methyl-5,6,7,8- tetrahydro-1H- naphtho[2,3- d]imidazole-6- carboxamide |
| 124 | 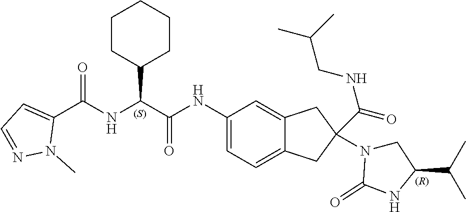 | N-((1S)-1-cyclohexyl- 2-((2- (isobutylcarbamoyl)-2- ((R)-4-isopropyl-2- oxoimidazolidin-1-yl)- 2,3-dihydro-1H-inden- 5-yl)amino)-2- oxoethyl)-1-methyl-1H- pyrazole-5-carboxamide |
| 125 | 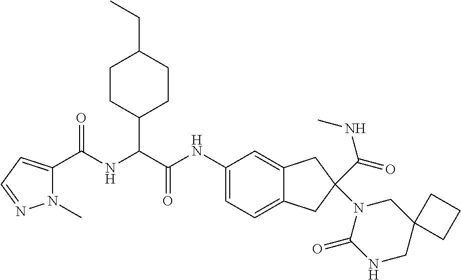 [SMILES: CCC1CCC(C(NC(=O)c2ccnn2C)C(=O)Nc2ccc3c(c2)CC(C(=O)NC)(N2CC4(CCC4)CNC2=O)C3)CC1] | N-(1-(4- ethylcyclohexyl)-2-((2- (methylcarbamoyl)-2- (7-oxo-6,8- diazaspiro[3.5]nonan-6- yl)-2,3-dihydro-1H- inden-5-yl)amino)-2- oxoethyl)-1-methyl-1H- pyrazole-5-carboxamide |
| 126 | 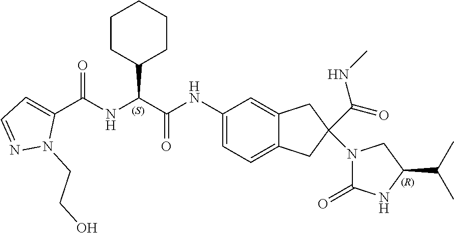 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-(2-hydroxyethyl)-1H- pyrazole-5-carboxamide |
| 127 | 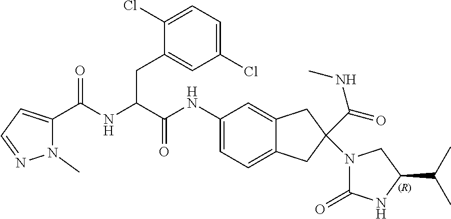 | N-(3-(2,5- dichlorophenyl)-1-((2- ((R)-4-isopropyl-2- oxoimidazolidin-1-yl)- 2-(methylcarbamoyl)- 2,3-dihydro-1H-inden- 5-yl)amino)-1- oxopropan-2-yl)-1- methyl-1H-pyrazole-5- carboxamide |
| 128 | 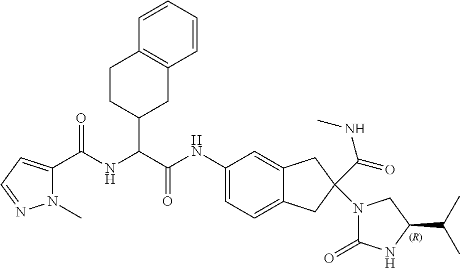 | N-(2-((2-((R)-4- isopropyl-2- oxoimidazolidin-1-yl)- 2-(methylcarbamoyl)- 2,3-dihydro-1H-inden- 5-yl)amino)-2-oxo-1- (1,2,3,4- tetrahydronaphthalen-2- yl)ethyl)-1-methyl-1H- pyrazole-5-carboxamide |
| 129 | 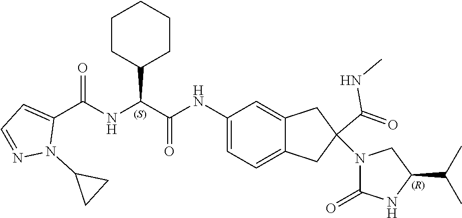 | N-((1S)-1-cyclohexyl- 2-((2-((R)-4-isopropyl- 2-oxoimidazolidin-1- yl)-2- (methylcarbamoyl)-2,3- dihydro-1H-inden-5- yl)amino)-2-oxoethyl)- 1-cyclopropyl-1H- pyrazole-5-carboxamide Chemical Formula: C32H43N704 |
| 130 | 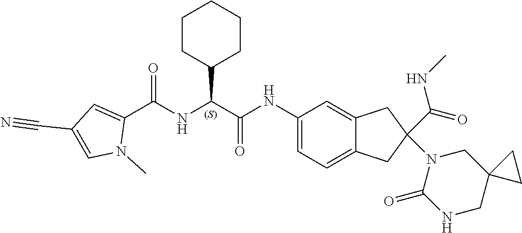 | 4-cyano-N-((1S)-1- cyclohexyl-2-((2- (methylcarbamoyl)-2- (6-oxo-5,7- diazaspiro[2.5]octan-5- yl)-2,3-dihydro-1H- inden-5-yl)amino)-2- oxoethyl)-1-methyl-1H- pyrrole-2-carboxamide |
| 131 | 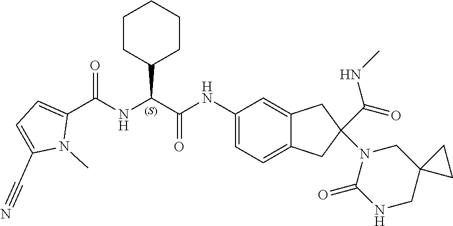 | 5-cyano-N-((1S)-1- cyclohexyl-2-((2- (methylcarbamoyl)-2- (6-oxo-5,7- diazaspiro[2.5]octan-5- yl)-2,3-dihydro-1H- inden-5-yl)amino)-2- oxoethyl)-1-methyl-1H- pyrrole-2-carboxamide |
| 132 | 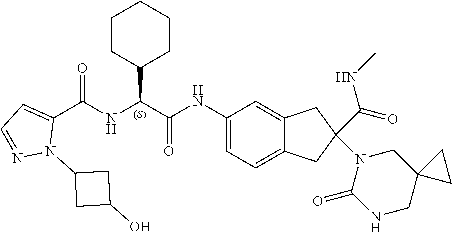 | N-((1S)-1-cyclohexyl- 2-((2- (methylcarbamoyl)-2- (6-oxo-5,7- diazaspiro[2.5]octan-5- yl)-2,3-dihydro-1H- inden-5-yl)amino)-2- oxoethyl)-1-(3- hydroxycyclobutyl)-1H- pyrazole-5-carboxamide |
| 133 | 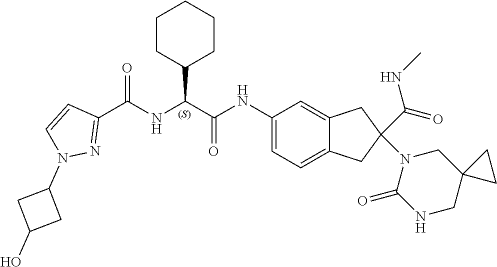 | N-((1S)-1-cyclohexyl- 2-((2- (methylcarbamoyl)-2- (6-oxo-5,7- diazaspiro[2.5]octan-5- yl)-2,3-dihydro-1H- inden-5-yl)amino)-2- oxoethyl)-1-(3- hydroxycyclobutyl)-1H- pyrazole-3-carboxamide |
| 134 | 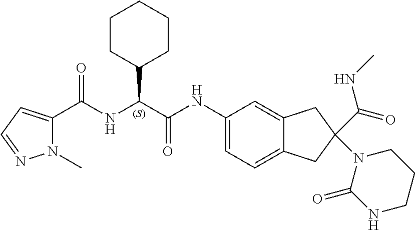 | N-((1S)-1-cyclohexyl- 2-((2- (methylcarbamoyl)-2- (2- oxotetrahydropyrimidin- 1(2H)-yl)-2,3-dihydro- 1H-inden-5-yl)amino)- 2-oxoethyl)-1-methyl- 1H-pyrazole-5- carboxamide |
| 135 | 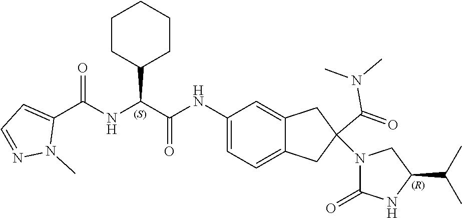 | N-((1S)-1-cyclohexyl- 2-((2- (dimethylcarbamoyl)-2- ((R)-4-isopropyl-2- oxoimidazolidin-1-yl)- 2,3-dihydro-1H-inden- 5-yl)amino)-2- oxoethyl)-1-methyl-1H- pyrazole-5-carboxamide |
| 136 | 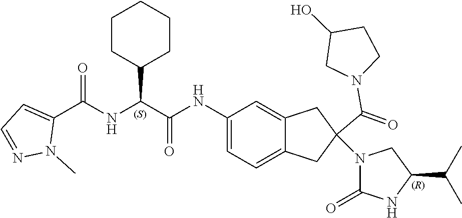 | N-((1S)-1-cyclohexyl- 2-((2-(3- hydroxypyrrolidine-1- carbonyl)-2-((R)-4- isopropyl-2- oxoimidazolidin-1-yl)- 2,3-dihydro-1H-inden- 5-yl)amino)-2- oxoethyl)-1-methyl-1H- pyrazole-5-carboxamide |
| 137 | 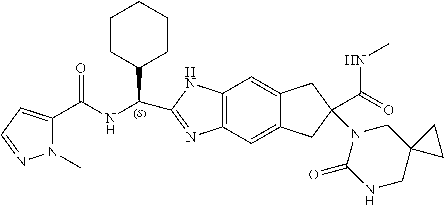 | 2-((S)-cyclohexyl(1- methyl-1H-pyrazole-5- carboxamido)methyl)- N-methyl-6-(6-oxo-5,7- diazaspiro[2.5]octan-5- yl)-1,5,6,7- tetrahydroindeno[5,6- d]imidazole-6- carboxamide |
| 138 | 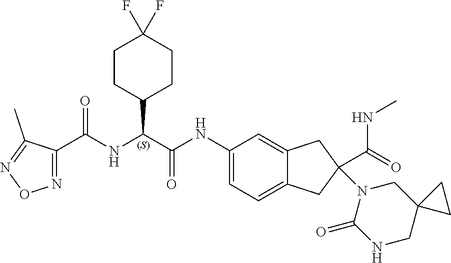 | N-((1S)-1-(4,4- difluorocyclohexyl)-2- ((2-(methylcarbamoyl)- 2-(6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 139 | 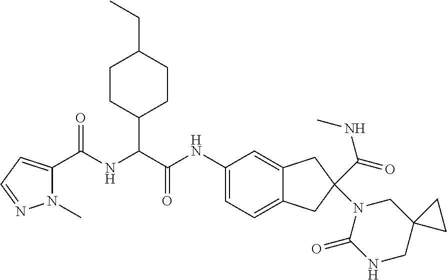 [SMILES: CCC1CCC(C(NC(=O)c2ccnn2C)C(=O)Nc2ccc3c(c2)CC(C(=O)NC)(N2CC4(CC4)CNC2=O)C3)CC1] | N-(1-(4- ethylcyclohexyl)-2-((2- (methylcarbamoyl)-2- (6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 140 | 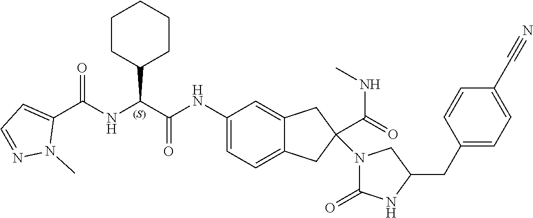 | N-((1S)-2-((2-(4-(4- cyanobenzyl)-2- oxoimidazolidin-1-yl)- 2-(methylcarbamoyl)- 2,3-dihydro-1H-inden- 5-yl)amino)-1- cyclohexyl-2-oxoethyl)- 1-methyl-1H-pyrazole- 5-carboxamide |
| 141 | 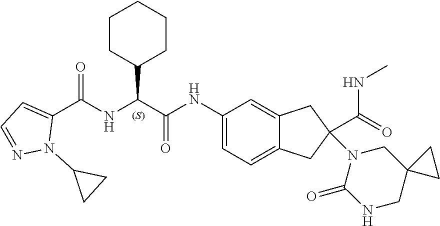 | N-((1S)-1-cyclohexyl- 2-((2- (methylcarbamoyl)-2- (6-oxo-5,7- diazaspiro[2.5]octan-5- yl)-2,3-dihydro-1H- inden-5-yl)amino)-2- oxoethyl)-1- cyclopropyl-1H- pyrazole-5-carboxamide |
| 142 | 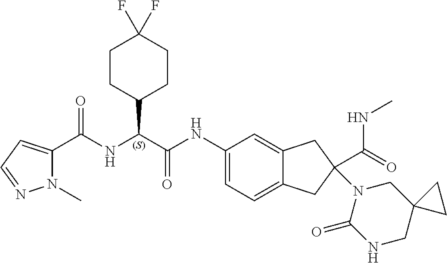 | N-((1S)-1-(4,4- difluorocyclohexyl)-2- ((2-(methylcarbamoyl)- 2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 143 | 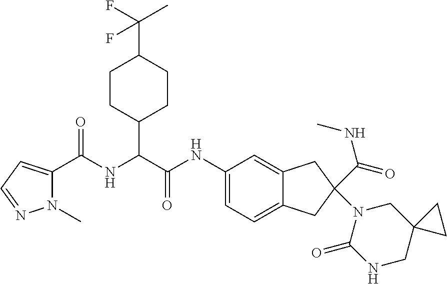 [SMILES: CNC(=O)C1(N2CC3(CC3)CNC2=O)Cc2ccc(NC(=O)C(NC(=O)c3ccnn3C)C3CCC(C(C)(F)F)CC3)cc2C1] | N-(1-(4-(1,1- difluoroethyl)cyclohexyl)- 2-((2- (methylcarbamoyl)-2- (6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 144 | 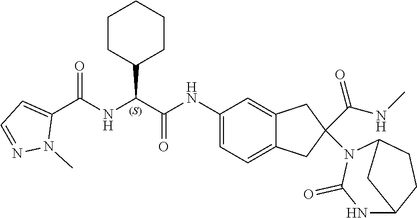 | N-((1S)-1-cyclohexyl-2- ((2-(methylcarbamoyl)- 2-(3-oxo-2,4- diazabicyclo[3.2.1]octan- 2-yl)-2,3-dihydro-1H- inden-5-yl)amino)-2- oxoethyl)-1-methyl-1H- pyrazole-5-carboxamide |
| 146 | 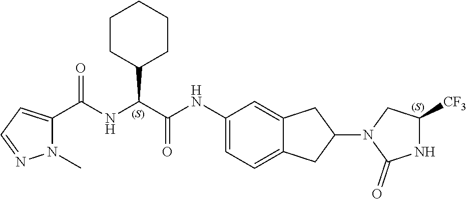 | N-((1S)-1-cyclohexyl- 2-oxo-2-((2-((S)-2-oxo- 4- (trifluoromethyl) imidazolidin-1-yl)-2,3- dihydro-1H-inden-5- yl)amino)ethyl)-1- methyl-1H-pyrazole-5- carboxamide |
| 147 | 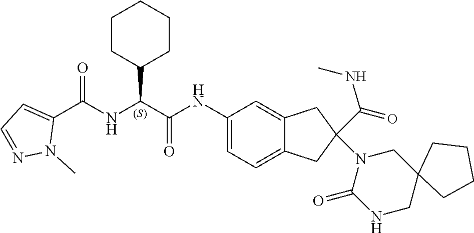 | N-((1S)-1-cyclohexyl- 2-((2- (methylcarbamoyl)-2- (8-oxo-7,9- diazaspiro[4.5]decan-7- yl)-2,3-dihydro-1H- inden-5-yl)amino)-2- oxoethyl)-1-methyl-1H- pyrazole-5-carboxamide |
| 148 | 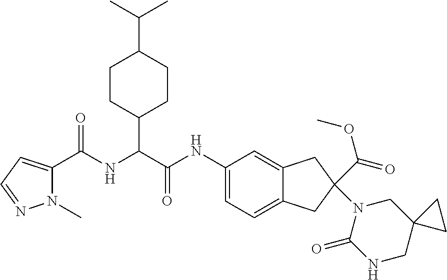 [SMILES: COC(=O)C1(N2CC3(CC3)CNC2=O)Cc2ccc(NC(=O)C(NC(=O)c3ccnn3C)C3CCC(C(C)C)CC3)cc2C1] | methyl 5-(2-(4- isopropylcyclohexyl)-2- (1-methyl-1H-pyrazole- 5- carboxamido)acetamido)- 2-(6-oxo-5,7- diazaspiro[2.5]octan-5- yl)-2,3-dihydro-1H- indene-2-carboxylate |
| 149 | 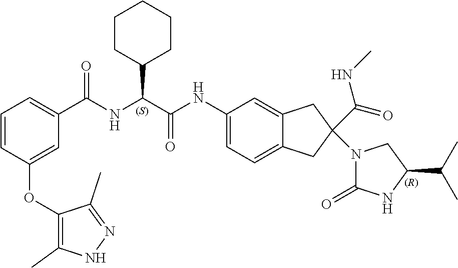 | 5-((S)-2-cyclohexyl-2- (3-((3,5-dimethyl-1H- pyrazol-4- yl)oxy)benzamido) acetamido)-2-((R)-4- isopropyl-2- oxoimidazolidin-1-yl)- N-methyl-2,3-dihydro- 1H-indene-2- carboxamide |
| 150 | 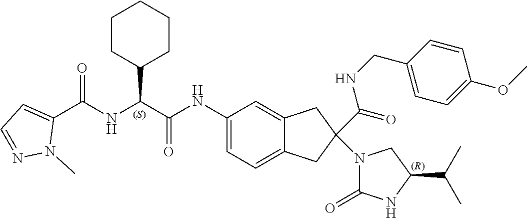 | N-((1S)-1-cyclohexyl- 2-((2-((R)-4-isopropyl- 2-oxoimidazolidin-1- yl)-2-((4- methoxybenzyl)carbam oyl)-2,3-dihydro-1H- inden-5-yl)amino)-2- oxoethyl)-1-methyl-1H- pyrazole-5-carboxamide |
| 151 | 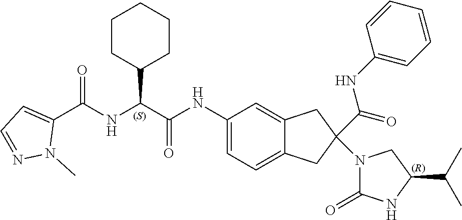 | N-((1S)-1-cyclohexyl- 2-((2-((R)-4-isopropyl- 2-oxoimidazolidin-1- yl)-2- (phenylcarbamoyl)-2,3- dihydro-1H-inden-5- yl)amino)-2-oxoethyl)- 1-methyl-1H-pyrazole- 5-carboxamide |
| 152 | 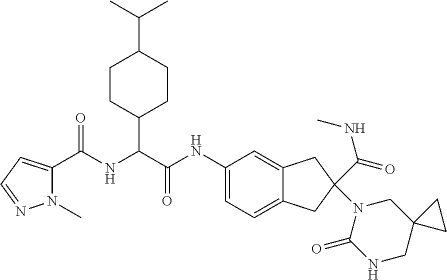 [SMILES: CNC(=O)C1(N2CC3(CC3)CNC2=O)Cc2ccc(NC(=O)C(NC(=O)c3ccnn3C)C3CCC(C(C)C)CC3)cc2C1] | N-(1-(4- isopropylcyclohexyl)-2- ((2-(methylcarbamoyl)- 2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 153 | 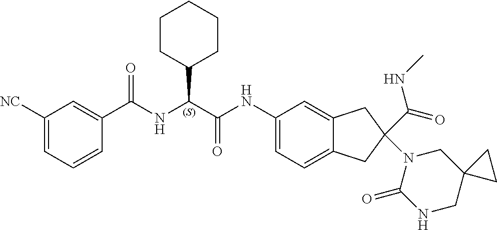 | 5-((S)-2-(3- cyanobenzamido)-2- cyclohexylacetamido)- N-methyl-2-(6-oxo-5,7- diazaspiro[2.5]octan-5- yl)-2,3-dihydro-1H- indene-2-carboxamide |
| 154 | 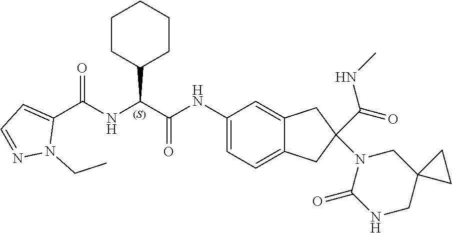 | N-((1S)-1-cyclohexyl- 2-((2- (methylcarbamoyl)-2- (6-oxo-5,7- diazaspiro[2.5]octan-5- yl)-2,3-dihydro-1H- inden-5-yl)amino)-2- oxoethyl)-1-ethyl-1H- pyrazole-5-carboxamide |
| 155 | 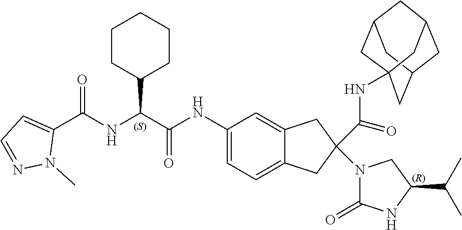 | N-((1S)-2-((2- (((3R,5R)-adamantan-1- yl)carbamoyl)-2-((R)-4- isopropyl-2- oxoimidazolidin-1-yl)- 2,3-dihydro-1H-inden- 5-yl)amino)-1- cyclohexyl-2-oxoethyl)- 1-methyl-1H-pyrazole- 5-carboxamide |
| 156 | 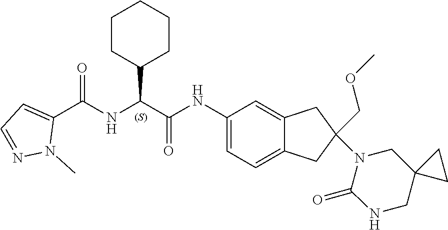 | N-((1S)-1-cyclohexyl- 2-((2-(methoxymethyl)- 2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 157 | 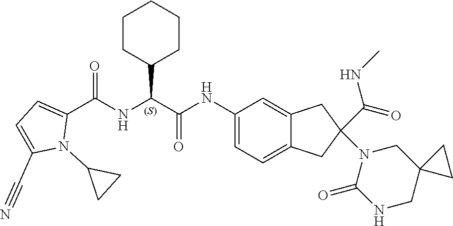 | 5-cyano-N-((1S)-1- cyclohexyl-2-((2- (methylcarbamoyl)-2- (6-oxo-5,7- diazaspiro[2.5]octan-5- yl)-2,3-dihydro-1H- inden-5-yl)amino)-2- oxoethyl)-1- cyclopropyl-1H- pyrrole-2-carboxamide |
| 158 | 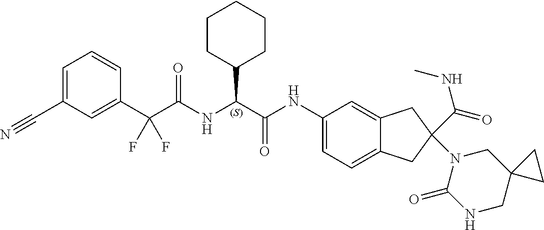 | 5-((S)-2-(2-(3- cyanophenyl)-2,2- difluoroacetamido)-2- cyclohexylacetamido)- N-methyl-2-(6-oxo-5,7- diazaspiro[2.5]octan-5- yl)-2,3-dihydro-1H- indene-2-carboxamide |
| 159 | 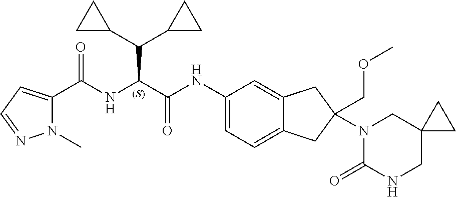 | N-((2S)-1,1- dicyclopropyl-3-((2- (methoxymethyl)-2-(6- oxo-5,7- diazaspiro[2.5]octan-5- yl)-2,3-dihydro-1H- inden-5-yl)amino)-3- oxopropan-2-yl)-1- methyl-1H-pyrazole-5- carboxamide |
| 160 | 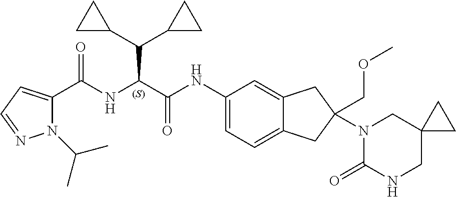 | N-((2S)-1,1- dicyclopropyl-3-((2- (methoxymethyl)-2-(6- oxo-5,7- diazaspiro[2.5]octan-5- yl)-2,3-dihydro-1H- inden-5-yl)amino)-3- oxopropan-2-yl)-1- isopropyl-1H-pyrazole- 5-carboxamide |
| 161 | 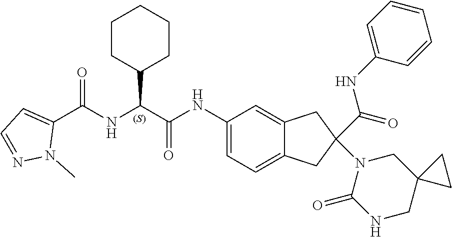 | N-((1S)-1-cyclohexyl-2- oxo-2-((2-(6-oxo-5,7- diazaspiro[2.5]octan-5- yl)-2- ((phenylamino)methyl)- 2,3-dihydro-1H-inden- 5-yl)amino)ethyl)-1- methyl-1H-pyrazole-5- carboxamide |
| 162 | 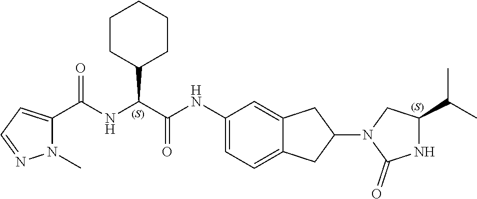 | N-((1S)-1-cyclohexyl- 2-((2-((R)-4-isopropyl- 2-oxoimidazolidin-1- yl)-2,3-dihydro-1H- inden-5-yl)amino)-2- oxoethyl)-1-methyl-1H- pyrazole-5-carboxamide |
| 163 | 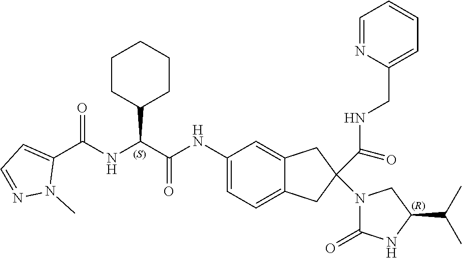 | N-((1S)-1-cyclohexyl- 2-((2-((R)-4-isopropyl- 2-oxoimidazolidin-1- yl)-2-((pyridin-2- ylmethyl)carbamoyl)- 2,3-dihydro-1H-inden- 5-yl)amino)-2- oxoethyl)-1-methyl-1H- pyrazole-5-carboxamide |
| 164 | 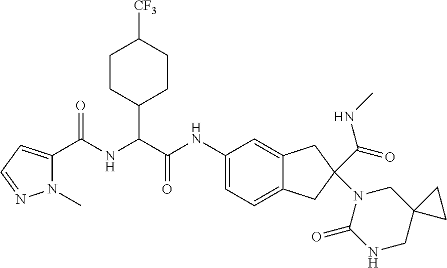 [SMILES: CNC(=O)C1(N2CC3(CC3)CNC2=O)Cc2ccc(NC(=O)C(NC(=O)c3ccnn3C)C3CCC(C(F)(F)F)CC3)cc2C1] | 1-methyl-N-(2-((2- (methylcarbamoyl)-2- (6-oxo-5,7- diazaspiro[2.5]octan-5- yl)-2,3-dihydro-1H- inden-5-yl)amino)-2- oxo-1-(4- (trifluoromethyl)cycloh exyl)ethyl)-1H- pyrazole-5-carboxamide |
| 165 | 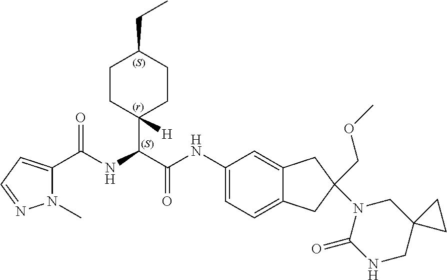 | N-((1S)-1-((1r,4S)-4- ethylcyclohexyl)-2-((2- (methoxymethyl)-2-(6- oxo-5,7- diazaspiro[2.5]octan-5- yl)-2,3-dihydro-1H- inden-5-yl)amino)-2- oxoethyl)-1-methyl-1H- pyrazole-5-carboxamide |
| 166 | 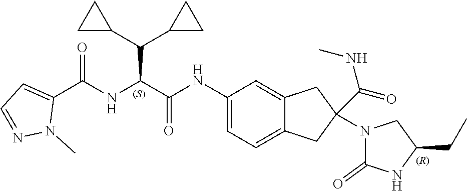 | N-((2S)-1,1- dicyclopropyl-3-((2- ((R)-4-ethyl-2- oxoimidazolidin-1-yl)- 2-(methylcarbamoyl)- 2,3-dihydro-1H-inden- 5-yl)amino)-3- oxopropan-2-yl)-1- methyl-1H-pyrazole-5- carboxamide |
| 167 | 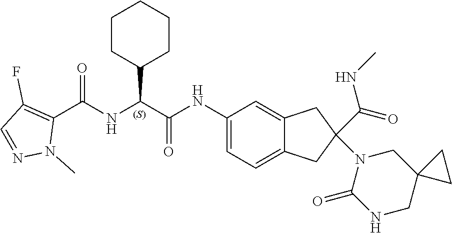 | N-((1S)-1-cyclohexyl- 2-((2- (methylcarbamoyl)-2- (6-oxo-5,7- diazaspiro[2.5]octan-5- yl)-2,3-dihydro-1H- inden-5-yl)amino)-2- oxoethyl)-4-fluoro-1- methyl-1H-pyrazole-5- carboxamide |
| 168 | 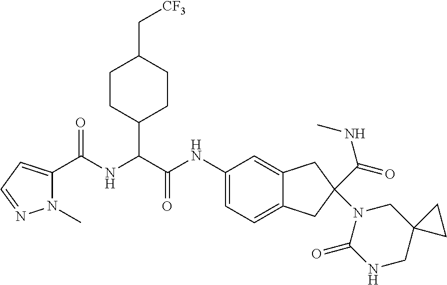 [SMILES: CNC(=O)C1(N2CC3(CC3)CNC2=O)Cc2ccc(NC(=O)C(NC(=O)c3ccnn3C)C3CCC(CC(F)(F)F)CC3)cc2C1] | 1-methyl-N-(2-((2- (methylcarbamoyl)-2- (6-oxo-5,7- diazaspiro[2.5]octan-5- yl)-2,3-dihydro-1H- inden-5-yl)amino)-2- oxo-1-(4-(2,2,2- trifluoroethyl)cyclohexyl) ethyl)-1H-pyrazole-5- carboxamide |
| 169 | 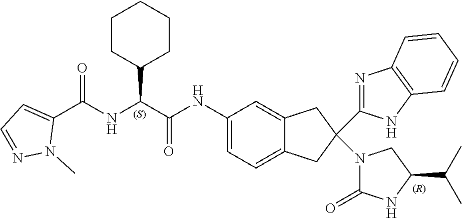 | N-((1S)-2-((2-(1H- benzo[d]imidazol-2-yl)- 2-((R)-4-isopropyl-2- oxoimidazolidin-1-yl)- 2,3-dihydro-1H-inden- 5-yl)amino)-1- cyclohexyl-2-oxoethyl)- 1-methyl-1H-pyrazole- 5-carboxamide |
| 171 | 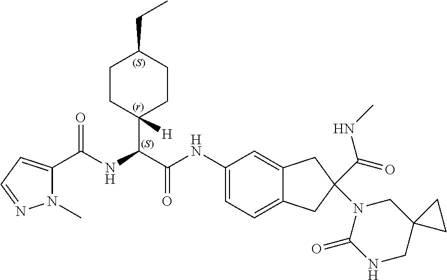 | N-((1S)-1-((1r,4S)-4- ethylcyclohexyl)-2-((2- (methylcarbamoyl)-2- (6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 172 | 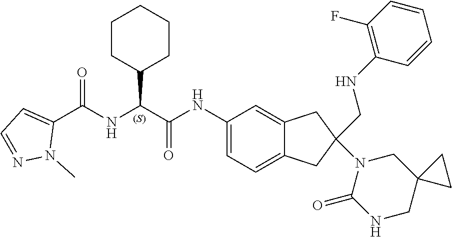 | N-((1S)-1-cyclohexyl-2- ((2-(((2- fluorophenyl)amino) methyl)-2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 173 | 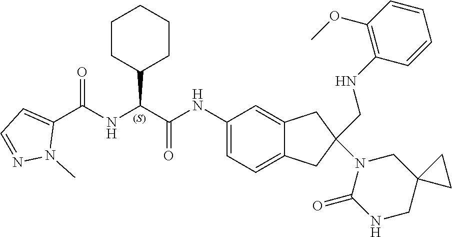 | N-((1S)-1-cyclohexyl-2- ((2-(((2- methoxyphenyl)amino) methyl)-2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 174 | 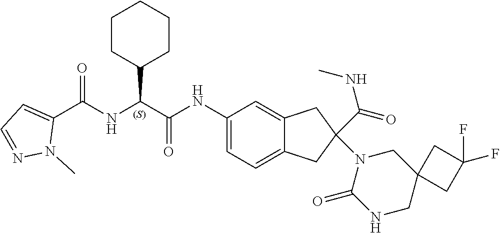 | N-((1S)-1-cyclohexyl- 2-((2-(2,2-difluoro-7- oxo-6,8- diazaspiro[3.5]nonan-6- yl)-2- (methylcarbamoyl)-2,3- dihydro-1H-inden-5- yl)amino)-2-oxoethyl)- 1-methyl-1H-pyrazole- 5-carboxamide |
| 175 | 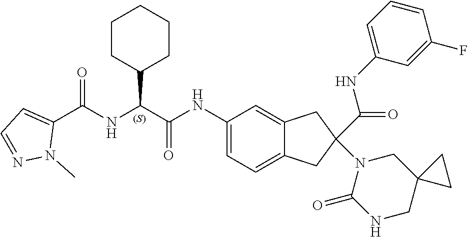 | N-((1S)-1-cyclohexyl- 2-((2-((2- fluorobenzyl)carbamoyl)- 2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 176 | 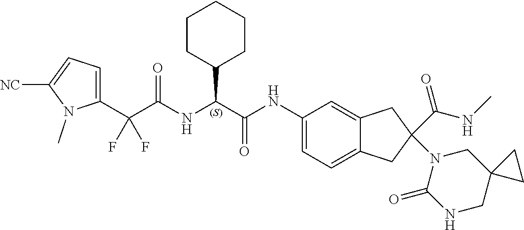 | 5-((S)-2-(2-(5-cyano-1- methyl-1H-pyrrol-2-yl)- 2,2-difluoroacetamido)- 2- cyclohexylacetamido)- N-methyl-2-(6-oxo-5,7- diazaspiro[2.5]octan-5- yl)-2,3-dihydro-1H- indene-2-carboxamide |
| 177 | 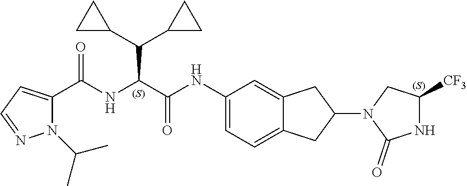 | N-((2S)-1,1- dicyclopropyl-3-oxo-3- ((2-((S)-2-oxo-4- (trifluoromethyl) imidazolidin- 1-yl)-2,3- dihydro-1H-inden-5- yl)amino)propan-2-yl)- 1-isopropyl-1H- pyrazole-5-carboxamide |
| 178 | 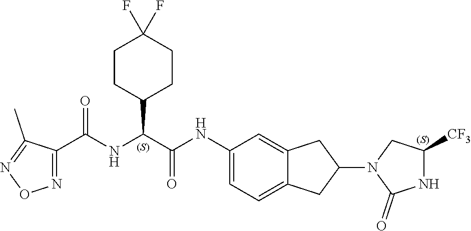 | N-((1S)-1-(4,4- difluorocyclohexyl)-2- oxo-2-((2-((S)-2-oxo-4- (trifluoromethyl) imidazolidin- 1-yl)-2,3- dihydro-1H-inden-5- yl)amino)ethyl)-4- methyl-1,2,5- oxadiazole-3- carboxamide |
| 179 | 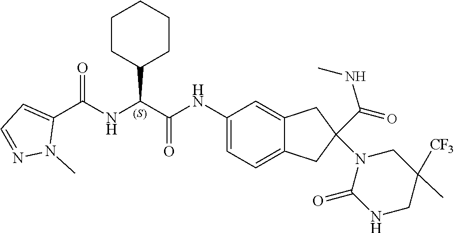 | N-((1S)-1-cyclohexyl-2- ((2-(5-methyl-2-oxo-5- (trifluoromethyl) tetrahydropyrimidin- 1(2H)-yl)- 2-(methylcarbamoyl)- 2,3-dihydro-1H-inden- 5-yl)amino)-2- oxoethyl)-1-methyl-1H- pyrazole-5-carboxamide |
| 180 | 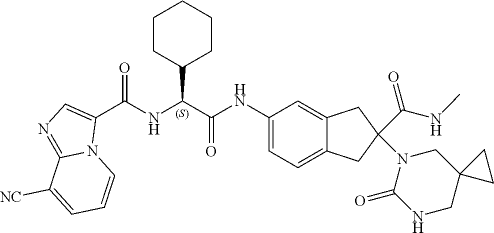 | 8-cyano-N-((1S)-1- cyclohexyl-2-((2- (methylcarbamoyl)-2- (6-oxo-5,7- diazaspiro[2.5]octan-5- yl)-2,3-dihydro-1H- inden-5-yl)amino)-2- oxoethyl)imidazo[1,2- a]pyridine-3- carboxamide |
| 181 | 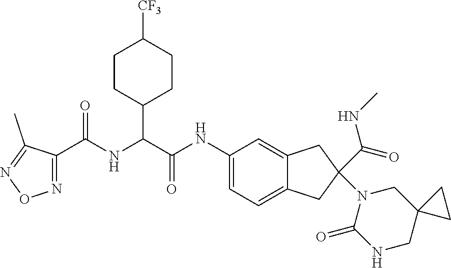 [SMILES: CNC(=O)C1(N2CC3(CC3)CNC2=O)Cc2ccc(NC(=O)C(NC(=O)c3nonc3C)C3CCC(C(F)(F)F)CC3)cc2C1] | 4-methyl-N-(2-((2- (methylcarbamoyl)-2- (6-oxo-5,7- diazaspiro[2.5]octan-5- yl)-2,3-dihydro-1H- inden-5-yl)amino)-2- oxo-1-(4- (trifluoromethyl) cyclohexyl) ethyl)-1,2,5- oxadiazole-3- carboxamide |
| 182 | 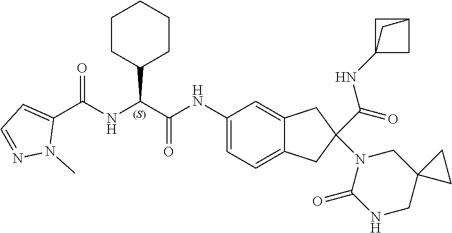 | N-((1S)-2-((2- (bicyclo[1.1.1]pentan-1- ylcarbamoyl)-2-(6-oxo- 5,7- diazaspiro[2.5]octan-5- yl)-2,3-dihydro-1H- inden-5-yl)amino)-1- cyclohexyl-2-oxoethyl)- 1-methyl-1H-pyrazole- 5-carboxamide |
| 183 | 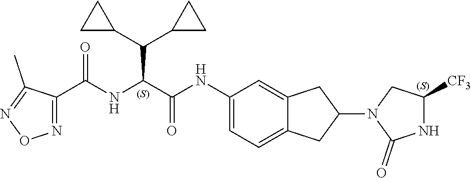 | N-((2S)-1,1- dicyclopropyl-3-oxo-3- ((2-((S)-2-oxo-4- (trifluoromethyl) imidazolidin- 1-yl)-2,3- dihydro-1H-inden-5- yl)amino)propan-2-yl)- 4-methyl-1,2,5- oxadiazole-3- carboxamide |
| 184 | 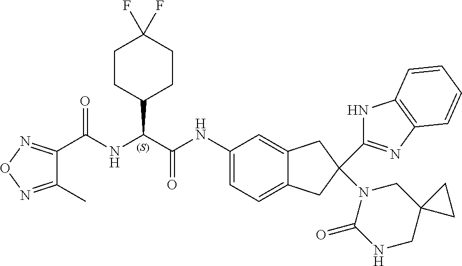 | N-((1S)-2-((2-(1H- benzo[d]imidazol-2-yl)- 2-(6-oxo-5,7- diazaspiro[2.5]octan-5- yl)-2,3-dihydro-1H- inden-5-yl)amino)-1- (4,4- difluorocyclohexyl)-2- oxoethyl)-4-methyl- 1,2,5-oxadiazole-3- carboxamide |
| 185 | 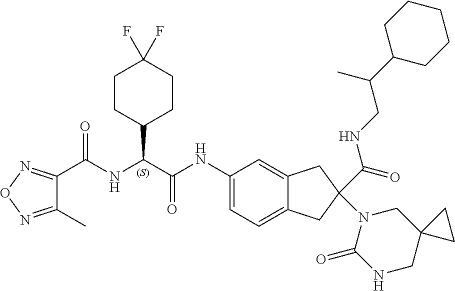 | N-((1S)-2-((2-((2- cyclohexylpropyl) carbamoyl)-2- (6-oxo-5,7- diazaspiro[2.5]octan-5- yl)-2,3-dihydro-1H- inden-5-yl)amino)-1- (4,4- difluorocyclohexyl)-2- oxoethyl)-4-methyl- 1,2,5-oxadiazole-3- carboxamide |
| 186 | 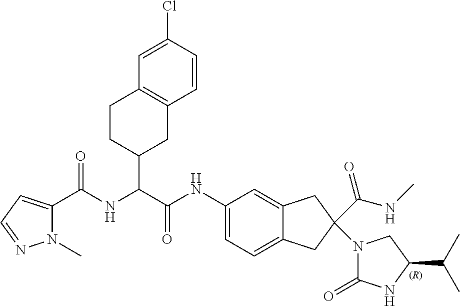 | N-(1-(6-chloro-1,2,3,4- tetrahydronaphthalen-2- yl)-2-((2-((R)-4- isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 187 | 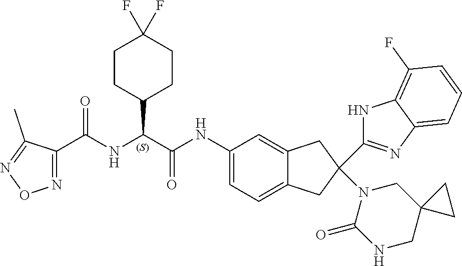 | N-((1S)-1-(4,4- difluorocyclohexyl)-2- ((2-(7-fluoro-1H- benzo[d]imidazol-2-yl)- 2-(6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 188 | 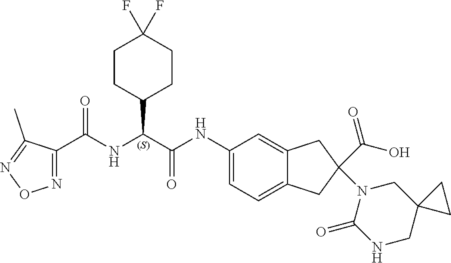 | 5-((S)-2-(4,4- difluorocyclohexyl)-2- (4-methyl-1,2,5- oxadiazole-3- carboxamido) acetamido)- 2-(6-oxo-5,7- diazaspiro [2.5]octan-5- yl)-2,3-dihydro-1H- indene-2-carboxylic acid |
| 189 | 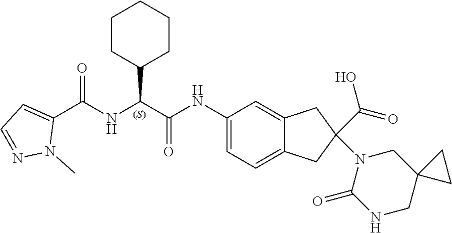 | 5-((S)-2-cyclohexyl-2- (1-methyl-1H-pyrazole- 5- carboxamido)acetamido)- 2-(6-oxo-5,7- diazaspiro[2.5]octan-5- yl)-2,3-dihydro-1H- indene-2-carboxylic acid |
| 190 | 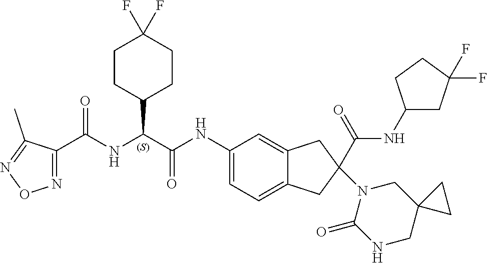 | N-((1S)-1-(4,4- difluorocyclohexyl)-2- ((2-((3,3- difluorocyclopentyl) carbamoyl)-2-(6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 191 | 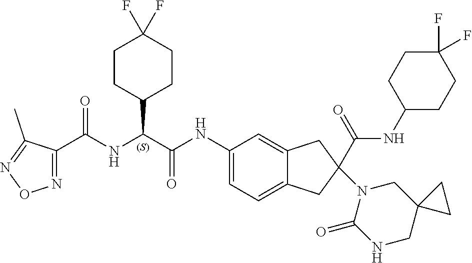 | N-((1S)-1-(4,4- difluorocyclohexyl)-2- ((2-((4,4- difluorocyclohexyl) carbamoyl)-2- (6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 192 | 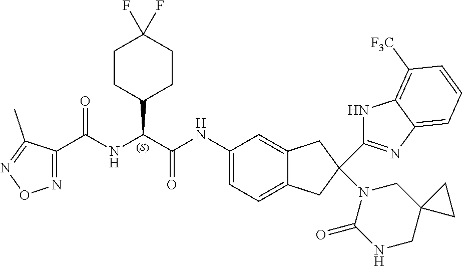 | N-((1S)-1-(4,4- difluorocyclohexyl)-2- oxo-2-((2-(6-oxo-5,7- diazaspiro[2.5]octan-5- yl)-2-(7- (trifluoromethyl)-1H- benzo[d]imidazol-2-yl)- 2,3-dihydro-1H-inden- 5-yl)amino)ethyl)-4- methyl-1,2,5- oxadiazole-3- carboxamide |
| 193 | 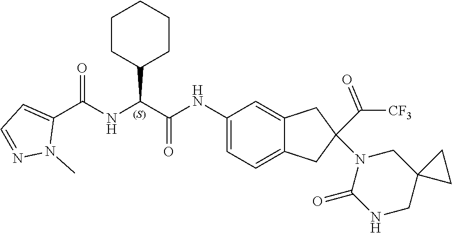 | N-((1S)-1-cyclohexyl-2- oxo-2-((2-(6-oxo-5,7- diazaspiro[2.5]octan-5- yl)-2-(2,2,2- trifluoroacetyl)-2,3- dihydro-1H-inden-5- yl)amino)ethyl)-1- methyl-1H-pyrazole-5- carboxamide |
| 194 | 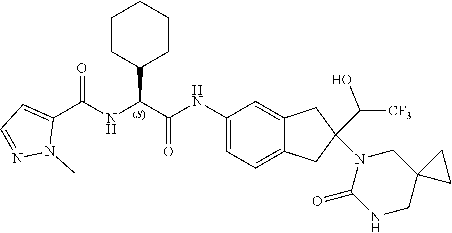 | N-((1S)-1-cyclohexyl-2- oxo-2-((2-(6-oxo-5,7- diazaspiro[2.5]octan-5- yl)-2-(2,2,2-trifluoro-1- hydroxyethyl)-2,3- dihydro-1H-inden-5- yl)amino)ethyl)-1- methyl-1H-pyrazole-5- carboxamide |
| 195 | 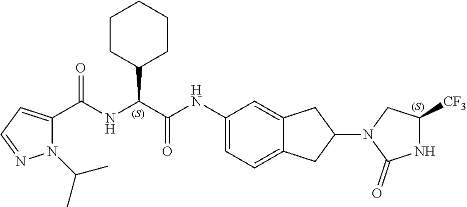 | N-((1S)-1-cyclohexyl- 2-oxo-2-((2-((S)-2-oxo- 4- (trifluoromethyl) imidazolidin- 1-yl)-2,3- dihydro-1H-inden-5- yl)amino)ethyl)-1- isopropyl-1H-pyrazole- 5-carboxamide |
| 196 | 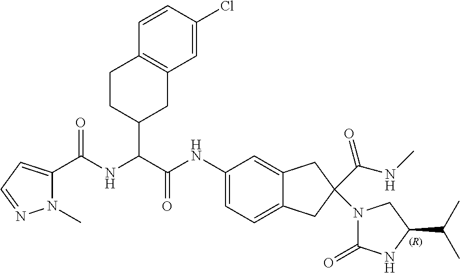 | N-(1-(7-chloro-1,2,3,4- tetrahydronaphthalen-2- yl)-2-((2-((R)-4- isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 197 | 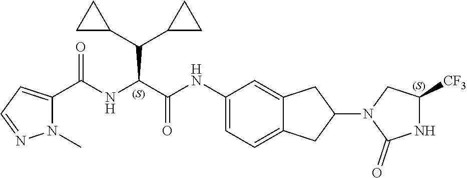 | N-((2S)-1,1- dicyclopropyl-3-oxo-3- ((2-((S)-2-oxo-4- (trifluoromethyl) imidazolidin-1-yl)-2,3- dihydro-1H-inden-5- yl)amino)propan-2-yl)- 1-methyl-1H-pyrazole- 5-carboxamide |
| 198 | 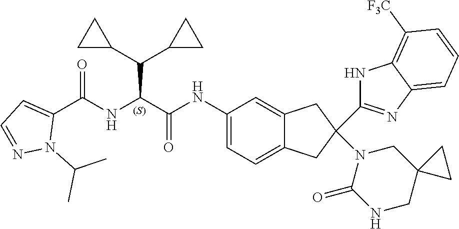 | N-((2S)-1,1- dicyclopropyl-3-oxo-3- ((2-(6-oxo-5,7- diazaspiro[2.5]octan-5- yl)-2-(7- (trifluoromethyl)-1H- benzo[d]imidazol-2-yl)- 2,3-dihydro-1H-inden- 5-yl)amino)propan-2- yl)-1-isopropyl-1H- pyrazole-5-carboxamide |
| 199 | 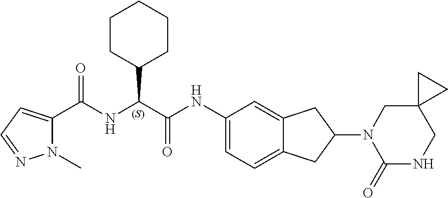 | N-((1S)-1-cyclohexyl- 2-oxo-2-((2-(6-oxo-5,7- diazaspiro[2.5]octan-5- yl)-2,3-dihydro-1H- inden-5- yl)amino)ethyl)-1- methyl-1H-pyrazole-5- carboxamide |
| 200 | 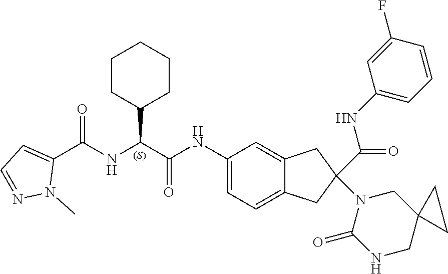 | N-((1S)-1-cyclohexyl- 2-((2-((3- fluorophenyl)carbamoyl)- 2-(6-oxo-5,7- diazaspiro[2.5]octan-5- yl)-2,3-dihydro-1H- inden-5-yl)amino)-2- oxoethyl)-1-methyl-1H- pyrazole-5-carboxamide |
| 201 | 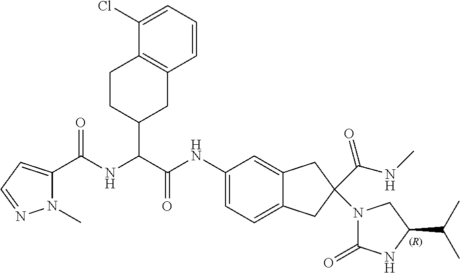 | N-(1-(5-chloro-1,2,3,4- tetrahydronaphthalen-2- yl)-2-((2-((R)-4- isopropyl-2- oxoimidazolidin-1-yl)- 2-(methylcarbamoyl)- 2,3-dihydro-1H-inden- 5-yl)amino)-2- oxoethyl)-1-methyl-1H- pyrazole-5-carboxamide |
| 202 | 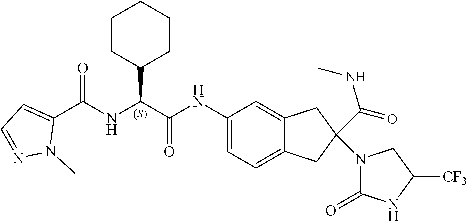 | N-((1S)-1-cyclohexyl- 2-((2- (methylcarbamoyl)-2- (2-oxo-4- (trifluoromethyl) imidazolidin-1-yl)-2,3- dihydro-1H-inden-5- yl)amino)-2-oxoethyl)- 1-methyl-1H-pyrazole- 5-carboxamide |
| 203 | 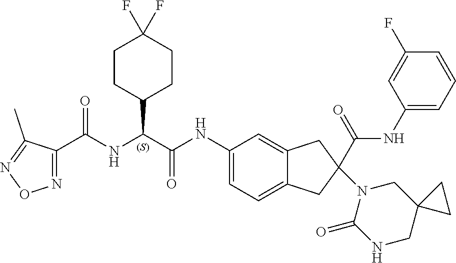 | N-((1S)-1-(4,4- difluorocyclohexyl)-2- ((2-((3- fluorophenyl) carbamoyl)- 2-(6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 204 | 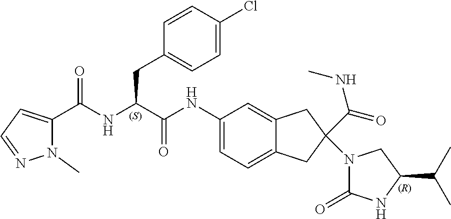 | N-((2S)-3-(4- chlorophenyl)-1-((2- ((R)-4-isopropyl-2- oxoimidazolidin-1-yl)- 2-(methylcarbamoyl)- 2,3-dihydro-1H-inden- 5-yl)amino)-1- oxopropan-2-yl)-1- methyl-1H-pyrazole-5- carboxamide |
| 205 | 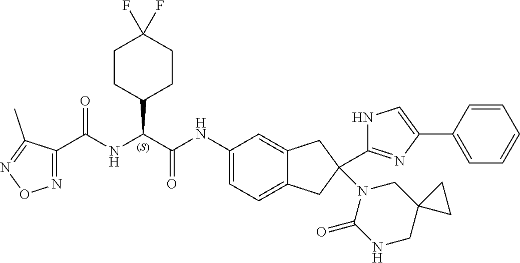 | N-((1S)-1-(4,4- difluorocyclohexyl)-2- oxo-2-((2-(6-oxo-5,7- diazaspiro[2.5]octan-5- yl)-2-(4-phenyl-1H- imidazol-2-yl)-2,3- dihydro-1H-inden-5- yl)amino)ethyl)-4- methyl-1,2,5- oxadiazole-3- carboxamide |
| 206 | 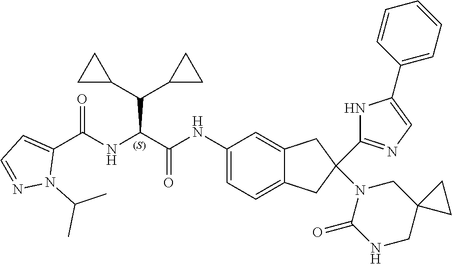 | N-((2S)-1,1- dicyclopropyl-3-oxo-3- ((2-(6-oxo-5,7- diazaspiro[2.5]octan-5- yl)-2-(5-phenyl-1H- imidazol-2-yl)-2,3- dihydro-1H-inden-5- yl)amino)propan-2-yl)- 1-isopropyl-1H- pyrazole-5-carboxamide |
| 207 | 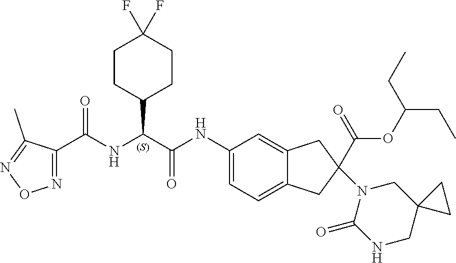 | pentan-3-yl 5-((S)-2- (4,4- difluorocyclohexyl)-2- (4-methyl-1,2,5- oxadiazole-3- carboxamido) acetamido)- 2-(6-oxo-5,7- diazaspiro[2.5]octan-5- yl)-2,3-dihydro-1H- indene-2-carboxylate |
| 208 | 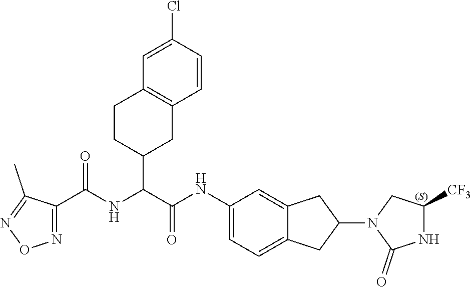 | N-(1-(6-chloro-1,2,3,4- tetrahydronaphthalen-2- yl)-2-oxo-2-((2-((S)-2- oxo-4- (trifluoromethyl) imidazolidin- 1-yl)-2,3- dihydro-1H-inden-5- yl)amino)ethyl)-4- methyl-1,2,5- oxadiazole-3- carboxamide |
| 209 | 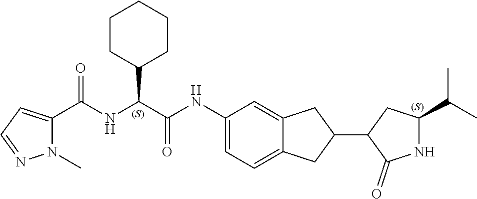 | N-((1S)-1-cyclohexyl- 2-((2-((5S)-5-isopropyl- 2-oxopyrrolidin-3-yl)- 2,3-dihydro-1H-inden- 5-yl)amino)-2- oxoethyl)-1-methyl-1H- pyrazole-5-carboxamide |
| 210 | 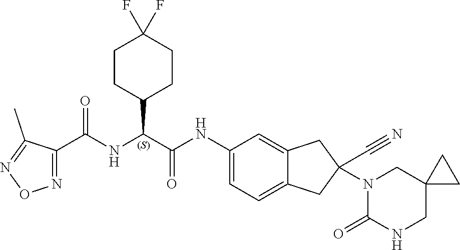 | N-((1S)-2-((2-cyano-2- (6-oxo-5,7- diazaspiro[2.5]octan-5- yl)-2,3-dihydro-1H- inden-5-yl)amino)-1- (4,4- difluorocyclohexyl)-2- oxoethyl)-4-methyl- 1,2,5-oxadiazole-3- carboxamide |
| 211 | 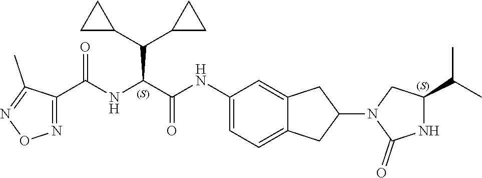 | N-((2S)-1,1- dicyclopropyl-3-((2- ((5S)-5-isopropyl-2- oxopyrrolidin-3-yl)-2,3- dihydro-1H-inden-5- yl)amino)-3-oxopropan- 2-yl)-4-methyl-1,2,5- oxadiazole-3- carboxamide |
| 213 | 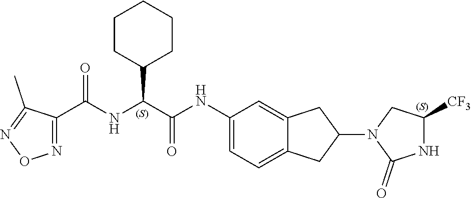 | N-((1S)-1-cyclohexyl- 2-oxo-2-((2-((S)-2-oxo- 4- (trifluoromethyl) imidazolidin-1-yl)-2,3- dihydro-1H-inden-5- yl)amino)ethyl)-4- methyl-1,2,5- oxadiazole-3- carboxamide |
| 214 | 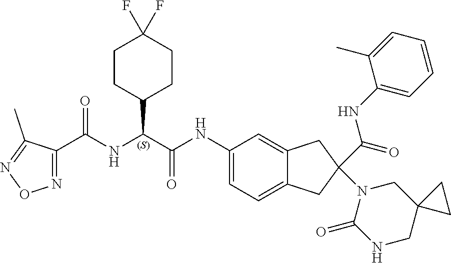 | N-((1S)-1-(4,4- difluorocyclohexyl)-2- oxo-2-((2-(6-oxo-5,7- diazaspiro[2.5]octan-5- yl)-2-(o- tolylcarbamoyl)-2,3- dihydro-1H-inden-5- yl)amino)ethyl)-4- methyl-1,2,5- oxadiazole-3- carboxamide |
| 215 | 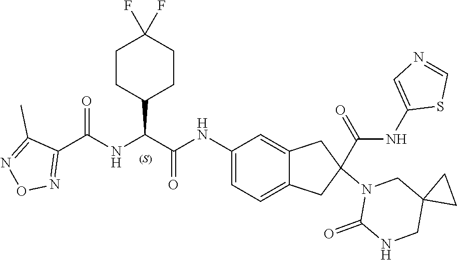 | N-((1S)-1-(4,4- difluorocyclohexyl)-2- oxo-2-((2-(6-oxo-5,7- diazaspiro[2.5]octan-5- yl)-2-(thiazol-5- ylcarbamoyl)-2,3- dihydro-1H-inden-5- yl)amino)ethyl)-4- methyl-1,2,5- oxadiazole-3- carboxamide |
| 216 | 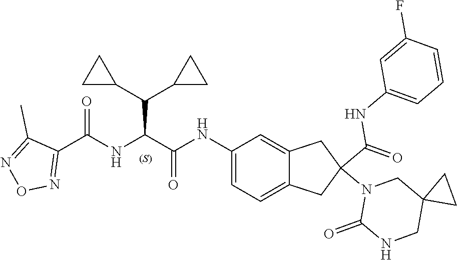 | N-((2S)-1,1- dicyclopropyl-3-((2-((3- fluorophenyl)carbamoyl)- 2-(6-oxo-5,7- diazaspiro[2.5]octan-5- yl)-2,3-dihydro-1H- inden-5-yl)amino)-3- oxopropan-2-yl)-4- methyl-1,2,5- oxadiazole-3- carboxamide |
| 217 | 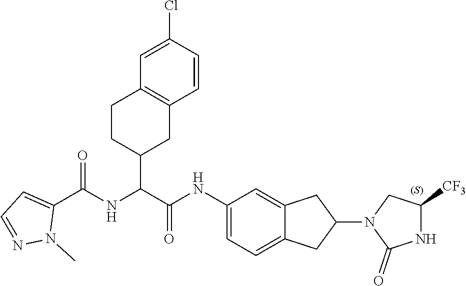 | N-(1-(6-chloro-1,2,3,4- tetrahydronaphthalen-2- yl)-2-oxo-2-((2-((S)-2- oxo-4- (trifluoromethyl) imidazolidin-1-yl)-2,3- dihydro-1H-inden-5- yl)amino)ethyl)-1- methyl-1H-pyrazole-5- carboxamide |
| 218 | 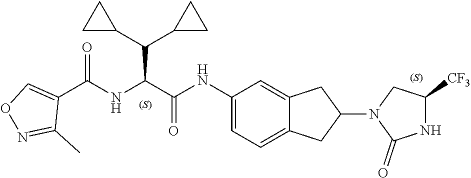 | N-((2S)-1,1- dicyclopropyl-3-oxo-3- ((2-((S)-2-oxo-4- (trifluoromethyl) imidazolidin-1-yl)-2,3- dihydro-1H-inden-5- yl)amino)propan-2-yl)- 3-methylisoxazole-4- carboxamide |
| 219 | 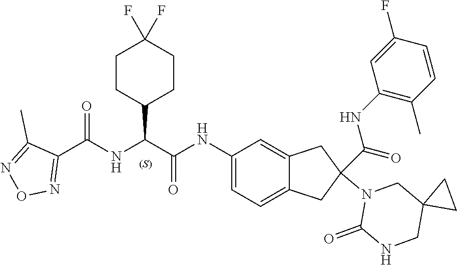 | N-((1S)-1-(4,4- difluorocyclohexyl)-2- ((2-((5-fluoro-2- methylphenyl)carbamoyl)- 2-(6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 220 | 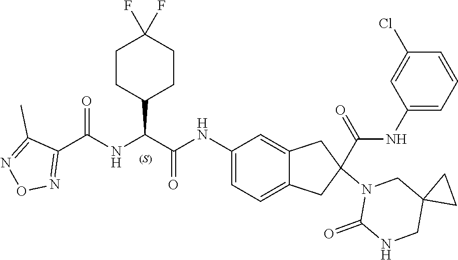 | N-((1S)-2-((2-((3- chlorophenyl)carbamoyl)- 2-(6-oxo-5,7- diazaspiro[2.5]octan-5- yl)-2,3-dihydro-1H- inden-5-yl)amino)-1- (4,4- difluorocyclohexyl)-2- oxoethyl)-4-methyl- 1,2,5-oxadiazole-3- carboxamide |
| 221 | 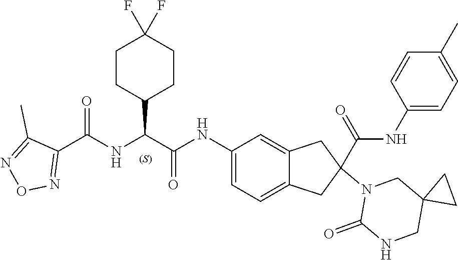 | N-((1S)-1-(4,4- difluorocyclohexyl)-2- oxo-2-((2-(6-oxo-5,7- diazaspiro[2.5]octan-5- yl)-2-(p- tolylcarbamoyl)-2,3- dihydro-1H-inden-5- yl)amino)ethyl)-4- methyl-1,2,5- oxadiazole-3- carboxamide |
| 222 | 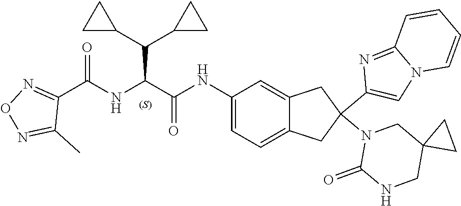 | N-((2S)-1,1- dicyclopropyl-3-((2- (imidazo[1,2-a]pyridin- 2-yl)-2-(6-oxo-5,7- diazaspiro[2.5]octan-5- yl)-2,3-dihydro-1H- inden-5-yl)amino)-3- oxopropan-2-yl)-4- methyl-1,2,5- oxadiazole-3- carboxamide |
| 223 | 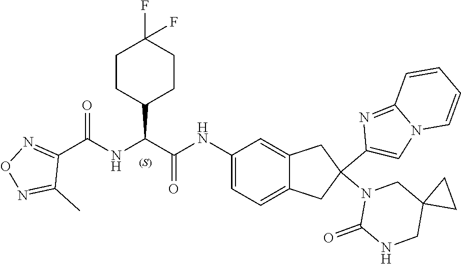 | N-((1S)-1-(4,4- difluorocyclohexyl)-2- ((2-(imidazo[1,2- a]pyridin-2-yl)-2-(6- oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 224 | 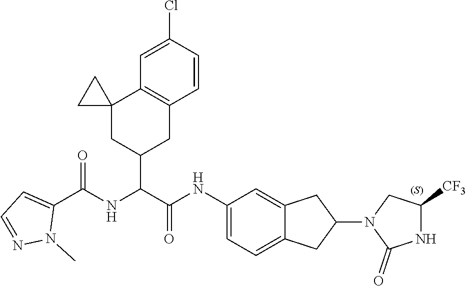 | N-(1-(7′-chloro-3′,4′- dihydro-2′H- spiro[cyclopropane-1,1′- naphthalen]-3′-yl)-2- oxo-2-((2-((S)-2-oxo-4- (trifluoromethyl) imidazolidin- 1-yl)-2,3- dihydro-1H-inden-5- yl)amino)ethyl)-1- methyl-1H-pyrazole-5- carboxamide |
| 225 | 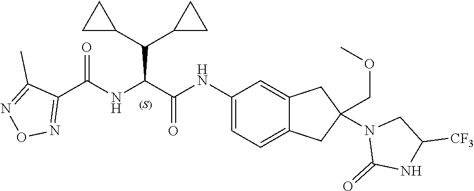 | N-((2S)-1,1- dicyclopropyl-3-((2- (methoxymethyl)-2-(2- oxo-4- (trifluoromethyl) imidazolidin- 1-yl)-2,3- dihydro-1H-inden-5- yl)amino)-3-oxopropan- 2-yl)-4-methyl-1,2,5- oxadiazole-3- carboxamide |
| 226 | 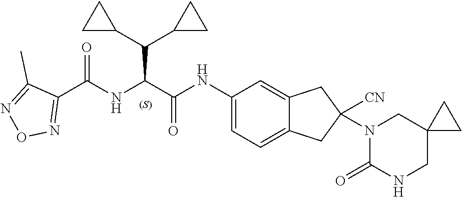 | N-((2S)-1-((2-cyano-2- (6-oxo-5,7- diazaspiro[2.5]octan-5- yl)-2,3-dihydro-1H- inden-5-yl)amino)-3,3- dicyclopropyl-1- oxopropan-2-yl)-4- methyl-1,2,5- oxadiazole-3- carboxamide |
| 227 | 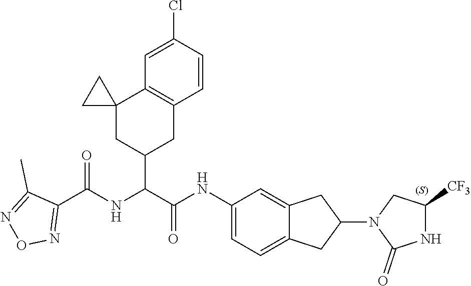 | N-(1-(7′-chloro-3′,4′- dihydro-2′H- spiro[cyclopropane-1,1′- naphthalen]-3′-yl)-2- oxo-2-((2-((S)-2-oxo-4- (trifluoromethyl) imidazolidin- 1-yl)-2,3- dihydro-1H-inden-5- yl)amino)ethyl)-4- methyl-1,2,5- oxadiazole-3- carboxamide |
| 228 | 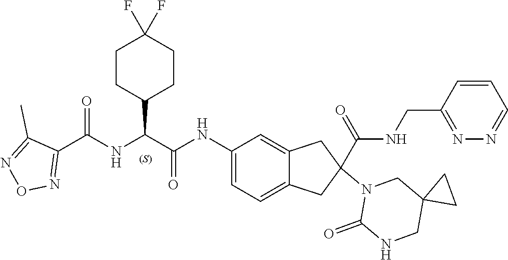 | N-((1S)-1-(4,4- difluorocyclohexyl)-2- oxo-2-((2-(6-oxo-5,7- diazaspiro[2.5]octan-5- yl)-2-((pyridazin-3- ylmethyl)carbamoyl)- 2,3-dihydro-1H-inden- 5-yl)amino)ethyl)-4- methyl-1,2,5- oxadiazole-3- carboxamide |
| 229 | 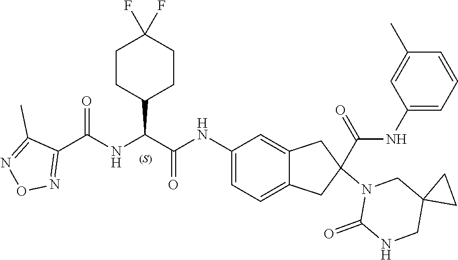 | N-((1S)-1-(4,4- difluorocyclohexyl)-2- oxo-2-((2-(6-oxo-5,7- diazaspiro[2.5]octan-5- yl)-2-(m- tolylcarbamoyl)-2,3- dihydro-1H-inden-5- yl)amino)ethyl)-4- methyl-1,2,5- oxadiazole-3- carboxamide |
| 230 | 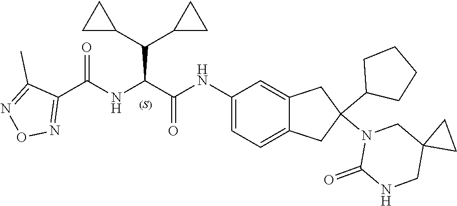 | N-((2S)-1-((2- cyclopentyl-2-(6-oxo- 5,7- diazaspiro[2.5]octan-5- yl)-2,3-dihydro-1H- inden-5-yl)amino)-3,3- dicyclopropyl-1- oxopropan-2-yl)-4- methyl-1,2,5- oxadiazole-3- carboxamide |
| 231 | 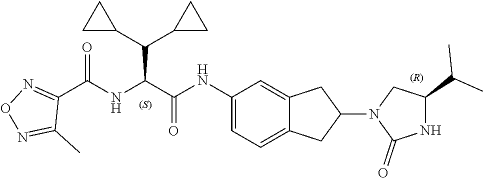 | N-((2S)-1,1- dicyclopropyl-3-((2- ((R)-4-isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 232 | 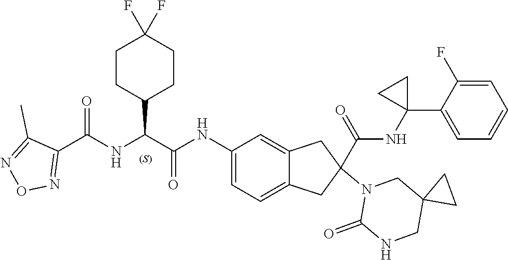 | N-((1S)-1-(4,4- difluorocyclohexyl)-2- ((2-((1-(2- fluorophenyl)cyclopropyl) carbamoyl)-2-(6- oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 233 | 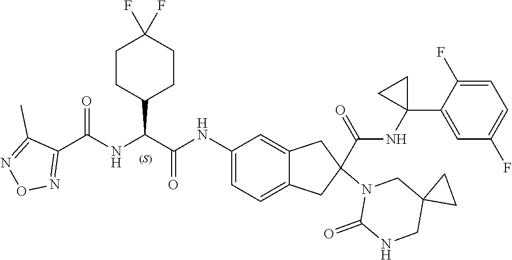 | N-((1S)-1-(4,4- difluorocyclohexyl)-2- ((2-((1-(2,5- difluorophenyl) cyclopropyl) carbamoyl)-2-(6- oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 234 | 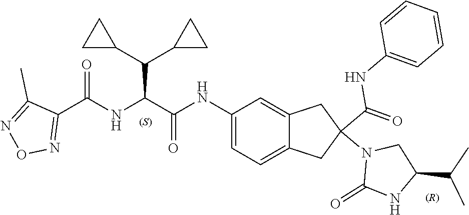 | N-((2S)-1,1- dicyclopropyl-3-((2- ((R)-4-isopropyl-2- oxoimidazolidin-1-yl)- 2-(phenylcarbamoyl)- 2,3-dihydro-1H-inden- 5-yl)amino)-3- oxopropan-2-yl)-4- methyl-1,2,5- oxadiazole-3- carboxamide |
| 235 | 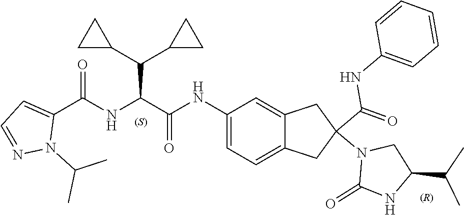 | N-((2S)-1,1- dicyclopropyl-3-((2- ((R)-4-isopropyl-2- oxoimidazolidin-1-yl)- 2-(phenylcarbamoyl)- 2,3-dihydro-1H-inden- 5-yl)amino)-3- oxopropan-2-yl)-1- isopropyl-1H-pyrazole- 5-carboxamide |
| 236 | 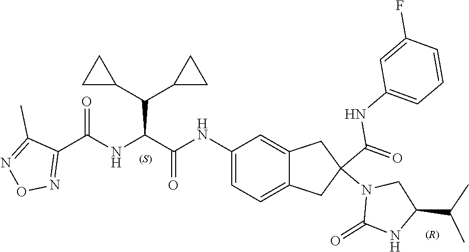 | N-((2S)-1,1- dicyclopropyl-3-((2-((3- fluorophenyl)carbamoyl)- 2-((R)-4-isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 237 | 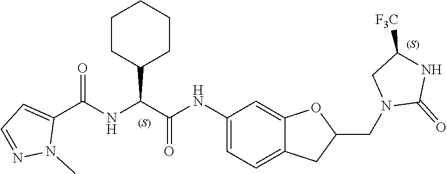 | N-((1S)-1-cyclohexyl- 2-oxo-2-((2-(((S)-2- oxo-4- (trifluoromethyl) imidazolidin-1-yl) methyl)-2,3- dihydrobenzofuran-6- yl)amino)ethyl)-1- methyl-1H-pyrazole-5- carboxamide |
| 239 | 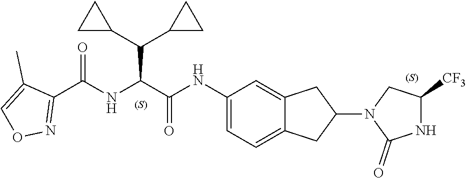 | N-((2S)-1,1- dicyclopropyl-3-oxo-3- ((2-((S)-2-oxo-4- (trifluoromethyl) imidazolidin-1-yl)-2,3- dihydro-1H-inden-5- yl)amino)propan-2-yl)- 4-methylisoxazole-3- carboxamide |
| 240 | 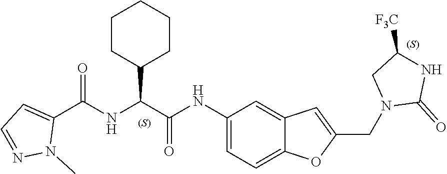 | N-((S)-1-cyclohexyl-2- oxo-2-((2-(((S)-2-oxo- 4- (trifluoromethyl) imidazolidin-1- yl)methyl)benzofuran- 5-yl)amino)ethyl)-1- methyl-1H-pyrazole-5- carboxamide |
| 241 | 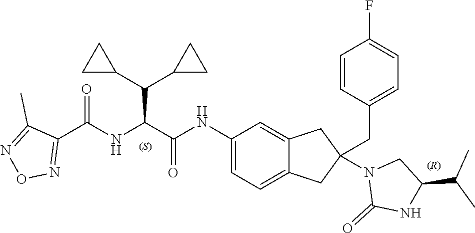 | N-((2S)-1,1- dicyclopropyl-3-((2-(4- fluorobenzyl)-2-((R)-4- isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 242 | 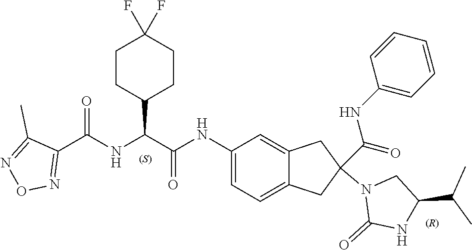 | N-((1S)-1-(4,4- difluorocyclohexyl)-2- ((2-((R)-4-isopropyl-2- oxoimidazolidin-1-yl)- 2-(phenylcarbamoyl)- 2,3-dihydro-1H-inden- 5-yl)amino)-2- oxoethyl)-4-methyl- 1,2,5-oxadiazole-3- carboxamide |
| 243 | 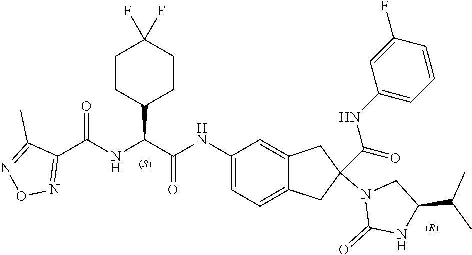 | N-((1S)-1-(4,4- difluorocyclohexyl)-2- ((2-((3- fluorophenyl)carbamoyl)- 2-((R)-4-isopropyl-2- oxoimidazolidin-1-yl)- 2,3-dihydro-1H-inden- 5-yl)amino)-2- oxoethyl)-4-methyl- 1,2,5-oxadiazole-3- carboxamide |
| 244 | 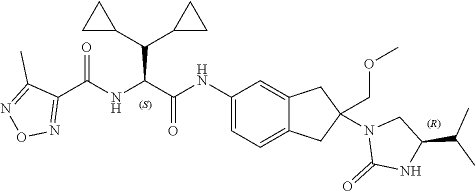 | N-((2S)-1,1- dicyclopropyl-3-((2- ((R)-4-isopropyl-2- oxoimidazolidin-1-yl)- 2-(methoxymethyl)-2,3- dihydro-1H-inden-5- yl)amino)-3-oxopropan- 2-yl)-4-methyl-1,2,5- oxadiazole-3- carboxamide |
| 245 | 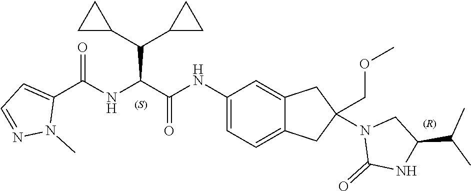 | N-((2S)-1,1- dicyclopropyl-3-((2- ((R)-4-isopropyl-2- oxoimidazolidin-1-yl)- 2-(methoxymethyl)-2,3- dihydro-1H-inden-5- yl)amino)-3-oxopropan- 2-yl)-1-methyl-1H- pyrazole-5-carboxamide |
| 246 | 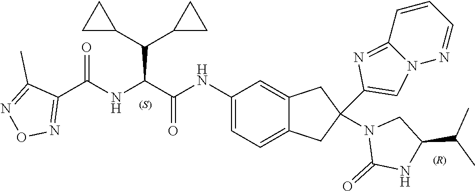 | N-((2S)-1,1- dicyclopropyl-3-((2- (imidazo[1,2- b]pyridazin-2-yl)-2- ((R)-4-isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 247 | 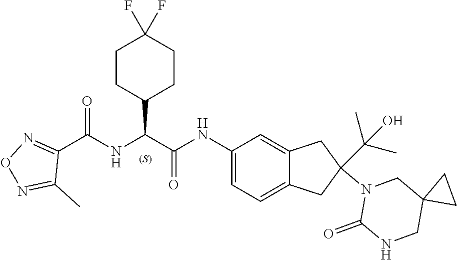 | N-((1S)-1-(4,4- difluorocyclohexyl)-2- ((2-(2-hydroxypropan- 2-yl)-2-(6-oxo-5,7- diazaspiro[2.5]octan-5- yl)-2,3-dihydro-1H- inden-5-yl)amino)-2- oxoethyl)-4-methyl- 1,2,5-oxadiazole-3- carboxamide |
| 248 | 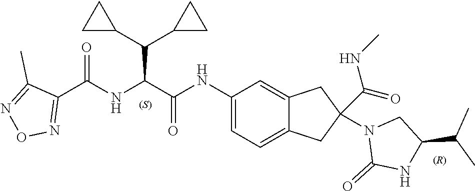 | N-((2S)-1,1- dicyclopropyl-3-((2- ((R)-4-isopropyl-2- oxoimidazolidin-1-yl)- 2-(methylcarbamoyl)- 2,3-dihydro-1H-inden- 5-yl)amino)-3- oxopropan-2-yl)-4- methyl-1,2,5- oxadiazole-3- carboxamide |
| 249 | 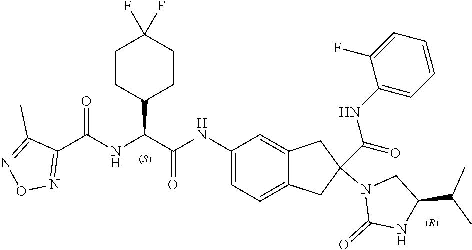 | N-((1S)-1-(4,4- difluorocyclohexyl)-2- ((2-((2- fluorophenyl)carbamoyl)- 2-((R)-4-isopropyl-2- oxoimidazolidin-1-yl)- 2,3-dihydro-1H-inden- 5-yl)amino)-2- oxoethyl)-4-methyl- 1,2,5-oxadiazole-3- carboxamide |
| 250 | 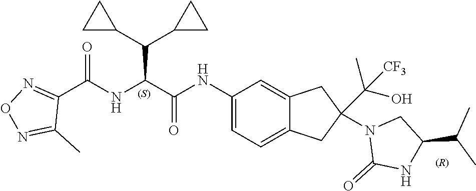 | N-((2S)-1,1- dicyclopropyl-3-((2- ((R)-4-isopropyl-2- oxoimidazolidin-1-yl)- 2-(1,1,1-trifluoro-2- hydroxypropan-2-yl)- 2,3-dihydro-1H-inden- 5-yl)amino)-3- oxopropan-2-yl)-4- methyl-1,2,5- oxadiazole-3- carboxamide |
| 251 | 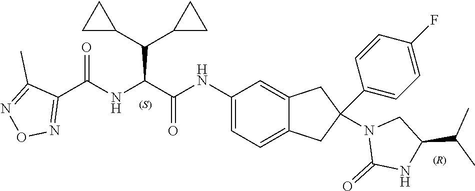 | N-((2S)-1,1- dicyclopropyl-3-((2-(4- fluorophenyl)-2-((R)-4- isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 252 | 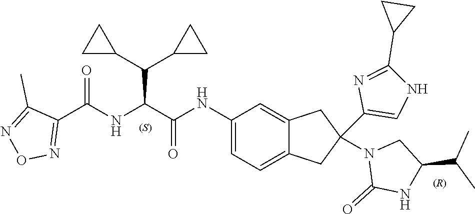 | N-((2S)-1,1- dicyclopropyl-3-((2-(2- cyclopropyl-1H- imidazol-4-yl)-2-((R)-4- isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 253 | 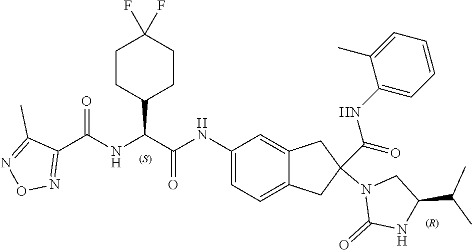 | N-((1S)-1-(4,4- difluorocyclohexyl)-2- ((2-((R)-4-isopropyl-2- oxoimidazolidin-1-yl)- 2-(o-tolylcarbamoyl)- 2,3-dihydro-1H-inden- 5-yl)amino)-2- oxoethyl)-4-methyl- 1,2,5-oxadiazole-3- carboxamide |
| 254 | 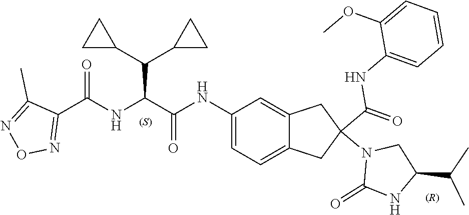 | N-((2S)-1,1- dicyclopropyl-3-((2- ((R)-4-isopropyl-2- oxoimidazolidin-1-yl)- 2-((2- methoxyphenyl) carbamoyl)- 2,3-dihydro-1H- inden-5-yl)amino)-3- oxopropan-2-yl)-4- methyl-1,2,5- oxadiazole-3- carboxamide |
| 255 | 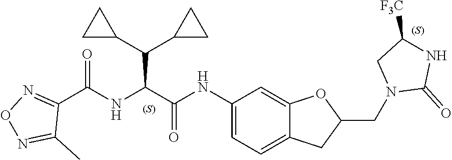 | N-((2S)-1,1- dicyclopropyl-3-oxo-3- ((2-(((S)-2-oxo-4- (trifluoromethyl) imidazolidin- 1-yl)methyl)-2,3- dihydrobenzofuran-6- yl)amino)propan-2-yl)- 4-methyl-1,2,5- oxadiazole-3- carboxamide |
| 257 | 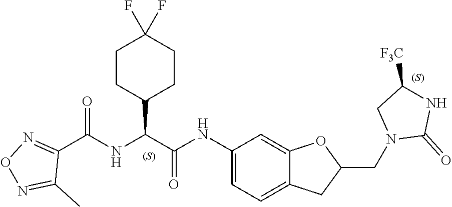 | N-((1S)-1-(4,4- difluorocyclohexyl)-2- oxo-2-((2-(((S)-2-oxo- 4- (trifluoromethyl) imidazolidin-1- yl)methyl)-2,3- dihydrobenzofuran-6- yl)amino)ethyl)-4- methyl-1,2,5- oxadiazole-3- carboxamide |
| 258 | 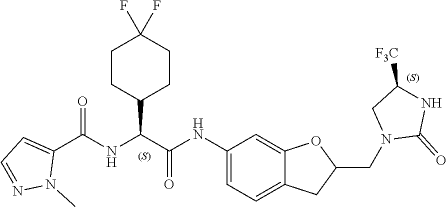 | N-((1S)-1-(4,4- difluorocyclohexyl)-2- oxo-2-((2-(((S)-2-oxo- 4- (trifluoromethyl) imidazolidin-1- yl)methyl)-2,3- dihydrobenzofuran-6- yl)amino)ethyl)-1- methyl-1H-pyrazole-5- carboxamide |
| 259 | 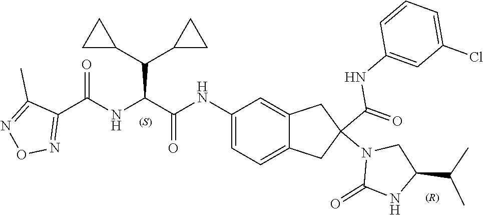 | N-((2S)-1-((2-((3- chlorophenyl) carbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 260 | 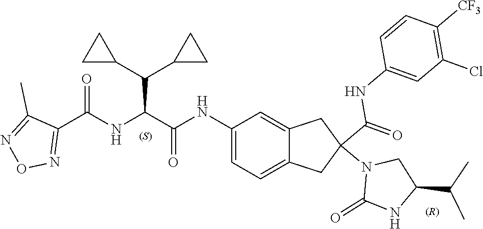 | N-((2S)-1-((2-((3- chloro-4- (trifluoromethyl)phenyl) carbamoyl)-2-((R)-4- isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 262 | 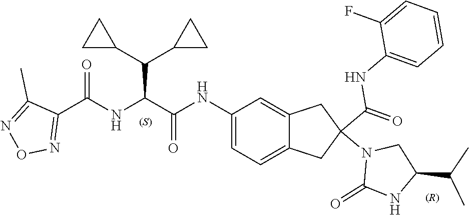 | N-((2S)-1,1- dicyclopropyl-3-((2-((2- fluorophenyl)carbamoyl)- 2-((R)-4-isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 263 | 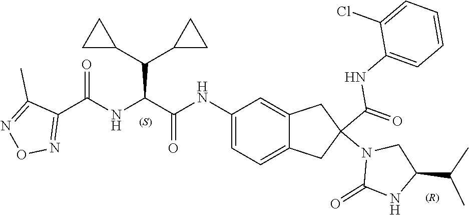 | N-((2S)-1-((2-((2- chlorophenyl) carbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 264 | 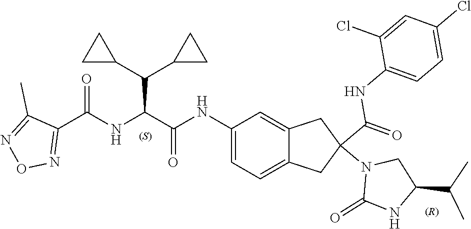 | N-((2S)-1,1- dicyclopropyl-3-((2- ((2,4- dichlorophenyl) carbamoyl)- 2-((R)-4-isopropyl- 2-oxoimidazolidin-1- yl)-2,3-dihydro-1H- inden-5-yl)amino)-3- oxopropan-2-yl)-4- methyl-1,2,5- oxadiazole-3- carboxamide |
| 265 | 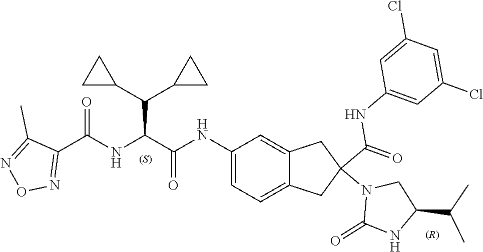 | N-((2S)-1,1- dicyclopropyl-3-((2- ((3,5- dichlorophenyl) carbamoyl)- 2-((R)-4-isopropyl- 2-oxoimidazolidin-1- yl)-2,3-dihydro-1H- inden-5-yl)amino)-3- oxopropan-2-yl)-4- methyl-1,2,5- oxadiazole-3- carboxamide |
| 266 | 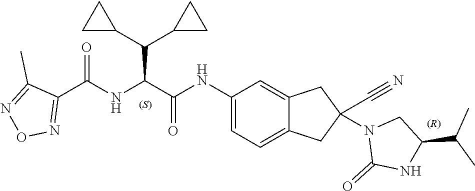 | N-((2S)-1-((2-cyano-2- ((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 267 | 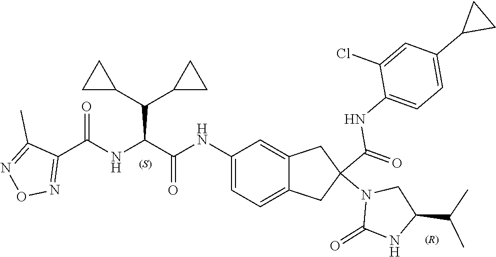 | N-((2S)-1-((2-((2- chloro-4- cyclopropylphenyl) carbamoyl)-2-((R)-4- isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 268 | 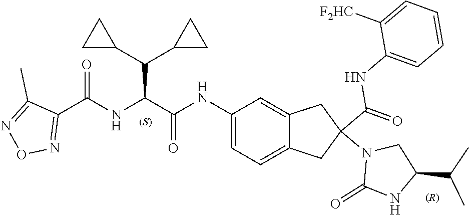 | N-((2S)-1,1- dicyclopropyl-3-((2-((2- (difluoromethyl)phenyl) carbamoyl)-2-((R)-4- isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 269 | 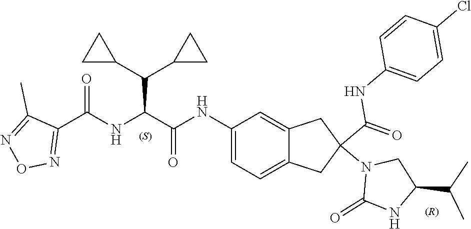 | N-((2S)-1-((2-((4- chlorophenyl)carbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 270 | 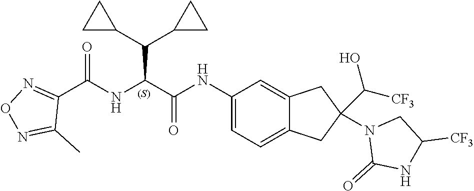 | N-((2S)-1,1- dicyclopropyl-3-oxo-3- ((2-(2-oxo-4- (trifluoromethyl) imidazolidin- 1-yl)-2-(2,2,2- trifluoro-1- hydroxyethyl)-2,3- dihydro-1H-inden-5- yl)amino)propan-2-yl)- 4-methyl-1,2,5- oxadiazole-3- carboxamide |
| 271 | 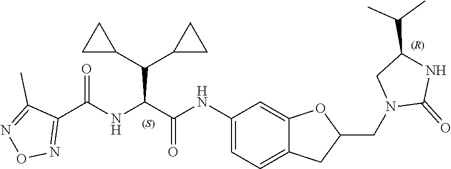 | N-((2S)-1,1- dicyclopropyl-3-((2- (((R)-4-isopropyl-2- oxoimidazolidin-1- yl)methyl)-2,3- dihydrobenzofuran-6- yl)amino)-3-oxopropan- 2-yl)-4-methyl-1,2,5- oxadiazole-3- carboxamide |
| 272 | 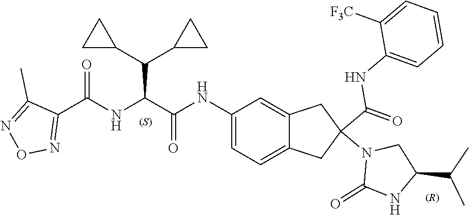 | N-((2S)-1,1- dicyclopropyl-3-((2- ((R)-4-isopropyl-2- oxoimidazolidin-1-yl)- 2-((2- (trifluoromethyl)phenyl) carbamoyl)-2,3- dihydro-1H-inden-5- yl)amino)-3-oxopropan- 2-yl)-4-methyl-1,2,5- oxadiazole-3- carboxamide |
| 273 | 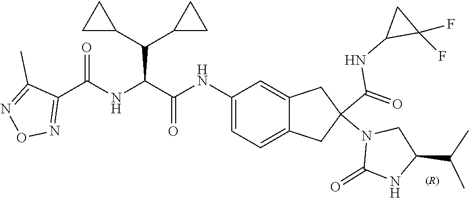 | N-((2S)-1,1- dicyclopropyl-3-((2- ((2,2- difluorocyclopropyl) carbamoyl)-2-((R)-4- isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 274 | 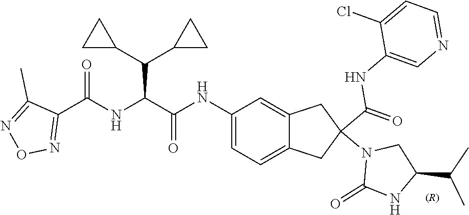 | N-((2S)-1-((2-((4- chloropyridin-3- yl)carbamoyl)-2-((R)-4- isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 275 | 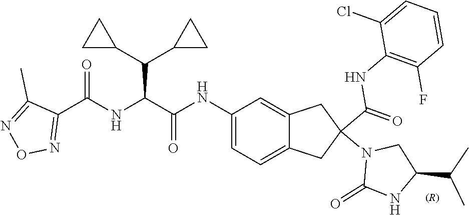 | N-((2S)-1-((2-((2- chloro-6- fluorophenyl)carbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 276 | 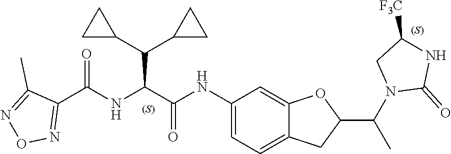 | N-((2S)-1,1- dicyclopropyl-3-oxo-3- ((2-(1-((S)-2-oxo-4- (trifluoromethyl) imidazolidin-1-yl) ethyl)-2,3- dihydrobenzofuran-6- yl)amino)propan-2-yl)- 4-methyl-1,2,5- oxadiazole-3- carboxamide |
| 277 | 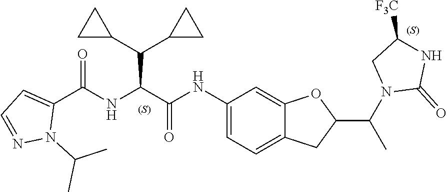 | N-((2S)-1,1- dicyclopropyl-3-oxo-3- ((2-(1-((S)-2-oxo-4- (trifluoromethyl) imidazolidin- 1-yl)ethyl)-2,3- dihydrobenzofuran-6- yl)amino)propan-2-yl)- 1-isopropyl-1H- pyrazole-5-carboxamide |
| 278 | 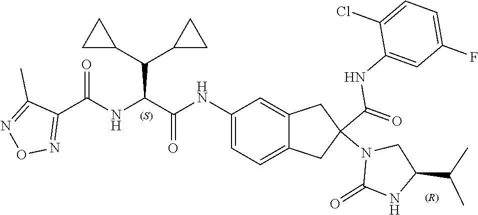 | N-((2S)-1-((2-((2- chloro-5- fluorophenyl)carbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 280 | 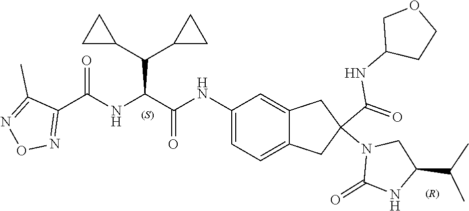 | N-((2S)-1,1- dicyclopropyl-3-((2- ((R)-4-isopropyl-2- oxoimidazolidin-1-yl)- 2-((tetrahydrofuran-3- yl)carbamoyl)-2,3- dihydro-1H-inden-5- yl)amino)-3-oxopropan- 2-yl)-4-methyl-1,2,5- oxadiazole-3- carboxamide |
| 281 | 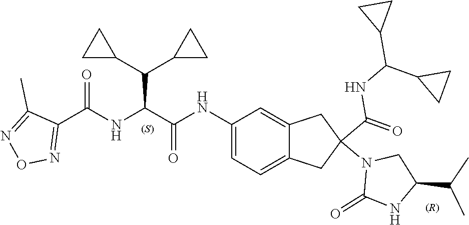 | N-((2S)-1,1- dicyclopropyl-3-((2- ((dicyclopropylmethyl) carbamoyl)-2-((R)-4- isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 282 | 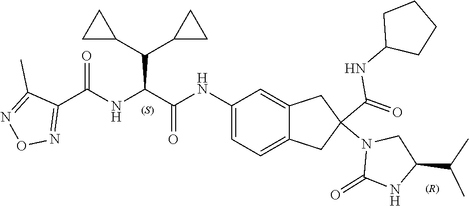 | N-((2S)-1-((2- (cyclopentylcarbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 283 | 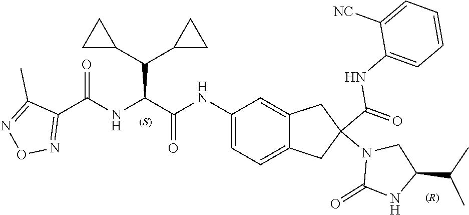 | N-((2S)-1-((2-((2- cyanophenyl)carbamoyl)- 2-((R)-4-isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 284 | 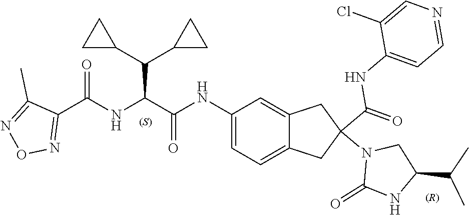 | N-((2S)-1-((2-((3- chloropyridin-4- yl)carbamoyl)-2-((R)-4- isopropyl-2- oxoimidazolidin-1-yl)- 2,3-dihydro-1H-inden- 5-yl)amino)-3,3- dicyclopropyl-1- oxopropan-2-yl)-4- methyl-1,2,5- oxadiazole-3- carboxamide |
| 285 | 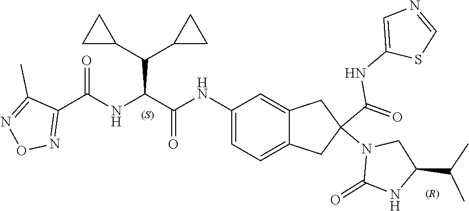 | N-((2S)-1,1- dicyclopropyl-3-((2- ((R)-4-isopropyl-2- oxoimidazolidin-1-yl)- 2-(thiazol-5- ylcarbamoyl)-2,3- dihydro-1H-inden-5- yl)amino)-3-oxopropan- 2-yl)-4-methyl-1,2,5- oxadiazole-3- carboxamide |
| 286 | 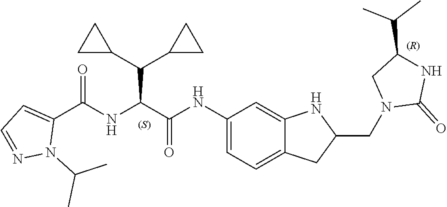 | N-((2S)-1,1- dicyclopropyl-3-((2- (((R)-4-isopropyl-2- oxoimidazolidin-1- yl)methyl)indolin-6- yl)amino)-3-oxopropan- 2-yl)-1-isopropyl-1H- pyrazole-5-carboxamide |
| 287 | 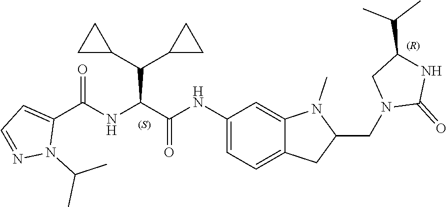 | N-((2S)-1,1- dicyclopropyl-3-((2- (((R)-4-isopropyl-2- oxoimidazolidin-1- yl)methyl)-1- methylindolin-6- yl)amino)-3-oxopropan- 2-yl)-1-isopropyl-1H- pyrazole-5-carboxamide |
| 288 | 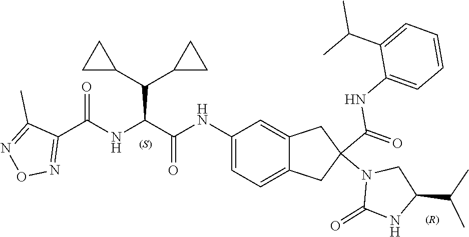 | N-((2S)-1,1- dicyclopropyl-3-((2- ((R)-4-isopropyl-2- oxoimidazolidin-1-yl)- 2-((2- isopropylphenyl) carbamoyl)-2, 3-dihydro-1H- inden-5-yl)amino)-3- oxopropan-2-yl)-4- methyl-1,2,5- oxadiazole-3- carboxamide |
| 289 | 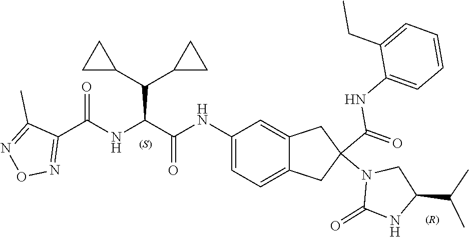 | N-((2S)-1,1- dicyclopropyl-3-((2-((2- ethylphenyl)carbamoyl)- 2-((R)-4-isopropyl-2- oxoimidazolidin-1-yl)- 2,3-dihydro-1H-inden- 5-yl)amino)-3- oxopropan-2-yl)-4- methyl-1,2,5- oxadiazole-3- carboxamide |
| 290 | 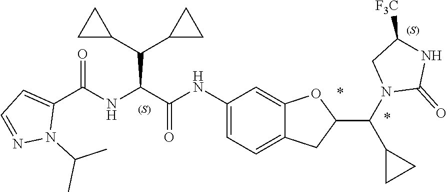 | N-((S)-1,1- dicyclopropyl-3-((2- (cyclopropyl((S)-2-oxo- 4- (trifluoromethyl) imidazolidin- 1-yl)-13-methyl)- 2,3-dihydro-213- benzofuran-6- yl)amino)-3-oxopropan- 2-yl)-1-isopropyl-1H- pyrazole-5-carboxamide |
| 291 | 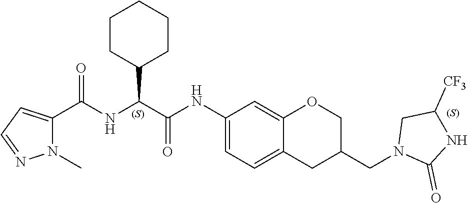 | N-((1S)-1-cyclohexyl- 2-oxo-2-((3-(((S)-2- oxo-4- (trifluoromethyl) imidazolidin-1- yl)methyl)chroman-7- yl)amino)ethyl)-1- methyl-1H-pyrazole-5- carboxamide |
| 292 | 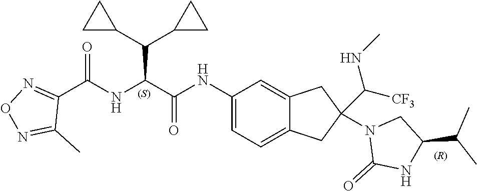 | N-((2S)-1,1- dicyclopropyl-3-((2- ((R)-4-isopropyl-2- oxoimidazolidin-1-yl)- 2-(2,2,2-trifluoro-1- (methylamino)ethyl)- 2,3-dihydro-1H-inden- 5-yl)amino)-3- oxopropan-2-yl)-4- methyl-1,2,5- oxadiazole-3- carboxamide |
| 293 | 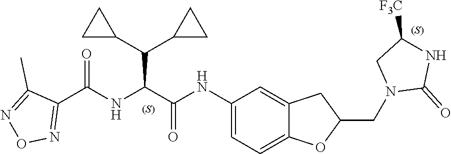 | N-((2S)-1,1- dicyclopropyl-3-oxo-3- ((2-(((S)-2-oxo-4- (trifluoromethyl) imidazolidin- 1-yl)methyl)-2,3- dihydrobenzofuran-5- yl)amino)propan-2-yl)- 4-methyl-1,2,5- oxadiazole-3- carboxamide |

| Ex. | Data |
|---|---|
| 1 | ¹H NMR (400 MHz, DMSO-d₆) δ ppm: 10.07 |
| | (s, 1H), 8.45 (d, J = 8.4 Hz, 1H), 7.54-7.25 (m, |
| | 4H), 7.10-7.05 (m, 1H), 7.01 (d, J = 1.6 Hz, |
| | 1H), 6.57 (s, 1H), 4.33 (t, J = 8.4 Hz, 1H), 3.98 |
| | (s, 3H), 3.55-3.36 (m, 4H), 3.33-3.05 (m, 2H), |
| | 2.76-2.69 (m, 1H), 2.54 (d, J = 4.0 Hz, 3H), |
| | (s, 3H), 3.55-3.36 (m, 4H), 3.33-3.05 (m, 2H), |
| | 2.76-2.69 (m, 1H), 2.54 (d, J = 4.0 Hz, 3H), |
| | 1.85-1.50 (m, 6H), 1.21-1.10 (m, 5H), 1.03- |
| | 0.95 (m, 3H). MS (ESI, m/e) [M + 1]⁺ 536.5. |
| 2 | ¹H NMR (400 MHz, DMSO-d₆) δ ppm: 10.05 |
| | (s, 1H), 8.44 (d, J = 8.4 Hz, 1H), 7.49-7.28 (m, |
| | 3H), 7.09-7.04 (m, 1H), 7.02 (d, J = 2.0 Hz, |
| | 1H), 6.35 (s, 1H), 4.33 (t, J = 8.4 Hz, 1H), 3.98 |
| | (s, 3H), 3.59-3.44 (m, 4H), 3.33-3.15 (m, 5H), |
| | 3.06-2.95 (m, 3H), 1.85-1.50 (m, 6H), 1.21- |
| | 1.10 (m, 5H), 1.01 (d, J = 6.0 Hz, 3H). MS |
| | (ESI, m/e) [M + 1]⁺ 523.3. |
| 3 | ¹H NMR (400 MHz, DMSO-d₆) δ ppm: 10.07 |
| | (s, 1H), 8.45 (d, J = 8.0 Hz, 1H), 7.59-7.28 (m, |
| | 3H), 7.11 (d, J = 8.0 Hz, 1H), 7.02 (d, J = 2.0 |
| | Hz, 1H), 6.43 (s, 1H), 4.59-4.50 (m, 1H), 4.33 |
| | (t, J = 8.4 Hz, 1H), 3.98 (s, 3H), 3.57-3.47 (m, |
| | 1H), 3.33-3.15 (m, 2H), 3.06-2.85 (m, 2H), |
| | 2.84-2.70 (m, 2H), 2.64-2.55 (m, 1H), 1.85- |
| | 1.50 (m, 6H), 1.21-1.10 (m, 4H), 1.03-0.95 (m, |
| | 3H). MS (ESI, m/e) [M + 1]⁺ 479.2. |
| 4 | ¹H NMR (400 MHz, DMSO-d₆) δ ppm: 10.04 |
| | (s, 1H), 8.42 (d, J = 8.0 Hz, 1H), 7.85-7.82 (m, |
| | 1H), 7.48 (d, J = 8.0 Hz, 1H), 7.42 (d, J = 4.0 |
| | Hz, 1H), 7.30-7.26 (m, 1H), 7.06 (d, J = 8.0 |
| | Hz, 1H), 7.02 (d, J = 4.0 Hz, 1H), 4.35-4.31 |
| | (m, 1H), 3.98 (s, 3H), 3.27 (s, 1H), 3.13-3.05 |
| | (m, 1H), 2.97-2.91 (m, 4H), 2.56 (d, J = 8.0 |
| | Hz, 1H), 1.79-1.77 (m, 2H), 1.68-1.65 (m, |
| | 2H), 1.58-1.51 (m, 2H), 1.19-1.11 (m, 4H). |
| | MS (ESI, m/e) [M + 1]⁺ 438.1. |
| 5 | ¹H NMR (400 MHz, DMSO-d₆) δ ppm: 12.23 |
| | (br, 1H), 8.74 (d, J = 8.8 Hz, 1H), 7.50-7.45 |
| | (m, 1H), 7.42 (d, J = 2.0 Hz, 1H), 7.30-7.20 |
| | (m, 2H), 7.02 (d, J = 1.6 Hz, 1H), 6.52 (s, 1H), |
| | 4.97 (t, J = 8.8 Hz, 1H), 3.97 (s, 3H), 3.59-3.40 |
| | (m, 4H), 3.32-3.15 (m, 2H), 2.73 (t, J = 7.6 Hz, |
| | 1H), 2.54 (d, J = 4.4 Hz, 3H), 2.11-2.00 (m, |
| | 1H), 1.85-1.78 (m, 1H), 1.72-1.52 (m, 3H), |
| | 1.38-1.30 (m, 1H), 1.21-0.95 (m, 9H). MS |
| | (ESI, m/e) [M + 1]⁺ 533.5. |
| 6 | ¹H NMR (400 MHz, DMSO-d₆) δ ppm: 10.10 |
| | (s, 1H), 8.46 (d, J = 8.2 Hz, 1H), 7.58 (d, J = |
| | 4.4 Hz, 1H), 7.41 (d, J = 2.0 Hz, 1H), 7.31 (s, |
| | 1H), 7.11-7.00 (m, 2H), 6.84 (d, J = 8.8 Hz, |
| | 1H), 6.60 (s, 1H), 4.50-4.25 (m, 5H), 3.98 (s, |
| | 3H), 3.74-3.63 (m, 2H), 3.27 (s, 3H), 3.18-3.12 |
| | (m, 1H), 2.55-2.53 (m, 3H), 1.78-1.75 (m, 2H), |
| | 1.65-1.54 (m, 4H), 1.20-1.05 (m, 6H). MS |
| | (ESI, m/e) [M + 1]⁺ 568.3. |
| 7 | ¹H NMR (400 MHz, DMSO-d₆) δ ppm: 10.01 |
| | (s, 1H), 8.42 (d, J = 8.4 Hz, 1H), 7.42 (d, J = |
| | 1.6 Hz, 1H), 7.39-7.27 (m, 3H), 7.04-6.97 (m, |
| | 2H), 6.40 (s, 1H), 4.32 (t, J = 8.8 Hz, 1H), 4.03 |
| | (s, 3H), 3.60-3.40 (m, 2H), 2.95- 2.50 (m, 8H), |
| | 2.38-2.20 (m, 1H), 1.90-1.50 (m, 8H), 1.21- |
| | 1.10 (m, 6H), 1.07-0.95 (m, 3H). MS (ESI, |
| | m/e) [M + 1]⁺ 564.3. |
| 8 | ¹H NMR (400 MHz, DMSO-d₆) δ ppm: 10.21 |
| | (s, 1H), 8.50 (d, J = 8.0 Hz, 1H), 7.80-7.76 (m, |
| | 1H), 7.57-7.28 (m, 4H), 7.06 (d, J = 2.0 Hz, |
| | 1H), 6.74 (d, J = 27.6 Hz, 1H), 4.37 (t, J = 8.4 |
| | Hz, 1H), 4.02 (s, 3H), 3.69-3.45 (m, 1H), 3.30- |
| | 2.72 (m, 5H), 2.55 (t, J = 4.8 Hz, 3H), 2.30- |
| | 1.91 (m, 2H), 1.90-1.55 (m, 6H), 1.21-1.10 (m, |
| | 5H), 1.07-0.95 (m, 3H). MS (ESI, m/e) [M + 1]⁺ |
| | 536.3. |
| 9 | MS (ESI) m/e [M + 1]⁺ 542.2 |
| 10 | ¹H NMR (400 MHz, DMSO-d₆) δ ppm: 10.06 |
| | (s, 1H), 8.47 (d, J = 8.0 Hz, 1H), 7.45-7.41 (m, |
| | 2H), 7.37-7.35 (m, 1H), 7.28-7.25 (m, 1H), |
| | 7.02-7.01 (m, 1H), 6.94 (d, J = 8.0 Hz, 1H), |
| | 6.38 (s, 1H), 4.36-4.31 (m, 1H), 3.46-3.41 (m, |
| | 1H), 3.24-3.18 (m, 2H), 3.08-2.83 (m, 5H), |
| | 2.56-2.53 (m, 5H), 1.98-1.92 (m, 1H), 1.79- |
| | 1.77 (m, 2H), 1.68-1.66 (m, 2H), 1.58-1.52 (m, |
| | 2H), 1.19-1.11 (m, 4H), 1.01-0.99 (m, 4H). |
| | MS (ESI, m/e) [M + 1]⁺ 550.3. |
| 11 | ¹H NMR (400 MHz, DMSO-d₆) δ ppm: 10.23 |
| | (s, 1H), 8.51 (d, J = 8.2 Hz, 1H), 7.99-7.94 (m, |
| | 1H), 7.52-7.36 (m, 3H), 7.10-7.03 (m, 1H), |
| | 6.75 (s, 1H), 6.69 (s, 1H), 4.47-4.29 (m, 3H), |
| | 4.03 (s, 3H), 3.70-3.59 (m, 1H), 3.48-3.40 (m, |
| | 1H), 2.89-2.78 (m, 1H), 1.95-1.77 (m, 2H), |
| | 1.77-1.55 (m, 4H), 1.30-1.22 (m, 5H), 1.12- |
| | 1.05 (m, 3H). MS (ESI, m/e) [M + 1]⁺ 493.3 |
| 12 | ¹H NMR (400 MHz, DMSO-d₆) δ ppm: 7.71 |
| | (s, 1H), 7.47 (s, 1H), 7.28 (d, J = 8.8 Hz, 1H), |
| | 7.18 (d, J = 8.8 Hz, 1H), 6.90 (d, J = 1.6 Hz, |
| | 1H), 6.31 (s, 1H), 4.48-4.41 (m, 2H), 4.09 (s, |
| | 3H), 3.79-3.75 (m, 1H), 3.52-3.47 (m, 1H), |
| | 3.00-2.88 (m, 1H), 1.94-1.72 (m, 5H), 1.41- |
| | 1.32 (m, 6H), 1.18 (d, J = 6.2 Hz, 4H). MS |
| | (ESI, m/e) [M + 1]⁺ 492.2. |
| 13 | ¹H NMR (400 MHz, DMSO-d₆) δ ppm: 10.12 |
| | (s, 1H), 8.49 (d, J = 8.0 Hz, 1H), 7.64-7.30 (m, |
| | 4H), 7.14-6.95 (m, 2H), 6.46 (s, 1H), 4.37 (t, |
| | J = 8.4 Hz, 1H), 4.02 (s, 3H), 3.50-3.40 (m, 2H), |
| | 3.33-3.09 (m, 6H), 2.58 (d, J = 4.4 Hz, 3H), |
| | 1.90-1.55 (m, 6H), 1.21-0.95 (m, 5H). MS |
| | (ESI, m/e) [M + 1]⁺ 522.4. |
| 14 | ¹H NMR (400 MHz, DMSO-d₆) δ ppm: 12.20 |
| | (d, J = 18.0 Hz, 1H), 9.40 (t, J = 6.4 Hz, 1H), |
| | 7.55-7.48 (m, 1H), 7.35 (d, J = 3.2 Hz, 1H), |
| | 7.27 (d, J = 4.0 Hz, 1H), 6.57 (s, 1H), 5.05 (t, |
| | J = 8.0 Hz, 1H), 3.65-3.40 (m, 4H), 3.32-3.20 |
| | (m, 2H), 2.83-2.69 (m, 1H), 2.58 (d, J = 4.4 |
| | Hz, 3H), 2.11-1.96 (m, 2H), 1.90-1.55 (m, |
| | 4H), 1.21-0.95 (m, 8H). MS (ESI, m/e) [M + 1]⁺ |
| | 535.4. |
| 15 | ¹H NMR (400 MHz, DMSO-d₆) δ ppm: 10.12 |
| | (s, 1H), 8.49 (d, J = 8.4 Hz, 1H), 7.57-7.30 (m, |
| | 3H), 7.14-6.95 (m, 4H), 6.63 (s, 1H), 4.38 (t, |
| | J = 8.8 Hz, 1H), 4.03 (s, 3H), 3.60-3.40 (m, 4H), |
| | 3.33-3.09 (m, 2H), 2.83-2.75 (m, 1H), 1.90- |
| | 1.55 (m, 6H), 1.21-1.10 (m, 5H), 1.07-0.95 (m, |
| | 3H). MS (ESI, m/e) [M + 1]⁺ 522.2. |
| 16 | ¹H NMR (400 MHz, DMSO-d₆) δ ppm: 10.03 |
| | (s, 1H), 8.58 (d, J = 8.0 Hz, 1H), 7.59-7.22 (m, |
| | 3H), 7.14-7.05 (m, 2H), 6.61 (s, 1H), 4.63-4.40 |
| | (m, 1H), 4.03 (s, 3H), 3.63-3.40 (m, 4H), 3.33- |
| | 3.05 (m, 2H), 2.87-2.69 (m, 1H), 2.58 (d, J = |
| | 4.4 Hz, 3H), 2.05-2.00 (m, 2H), 1.90-1.55 (m, |
| | 6H), 1.21-1.10 (m, 5H), 1.07-0.95 (m, 3H), |
| | 0.93-0.85 (m, 2H). MS (ESI, m/e) [M + 1]⁺ |
| | 550.2. |
| 17 | ¹H NMR (400 MHz, DMSO-d₆) δ ppm: 10.10 |
| | (s, 1H), 8.48 (d, J = 8.0 Hz, 1H), 7.77-7.30 (m, |
| | 4H), 7.14-7.05 (m, 2H), 6.48 (s, 1H), 4.38 (t, J = |
| | 8.0 Hz, 1H), 4.02 (s, 3H), 3.93-3.78 (m, 1H), |
| | 3.76-3.68 (m, 1H), 3.63-3.40 (m, 3H), 3.33- |
| | 3.05 (m, 1H), 2.88-2.76 (m, 1H), 2.58 (d, J = |
| | 4.4 Hz, 3H), 1.90-1.55 (m, 6H), 1.21-1.10 (m, |
| | 5H), 1.07-0.95 (m, 3H). MS (ESI, m/e) [M + 1]⁺ |
| | 536.3 |
| 18 | ¹H NMR (400 MHz, DMSO-d₆) δ ppm: 10.10 |
| | (s, 1H), 8.49 (d, J = 8.0 Hz, 1H), 7.58-7.29 (m, |
| | 4H), 7.12 (d, J = 7.6 Hz, 1H), 7.10 (s, 1H), |
| | 6.74 (s, 1H), 4.37 (t, J = 8.4 Hz, 1H), 4.02 (s, |
| | 3H), 3.55-3.35 (m, 4H), 3.33-3.11 (m, 2H), |
| | 2.90-2.83 (m, 1H), 2.58 (d, J = 4.4 Hz, 3H), |
| | 1.90-1.54 (m, 6H), 1.38-1.30 (m, 2H), 1.20- |
| | 0.95 (m, 5H), 0.74 (t, J = 7.2 Hz, 1H). MS |
| | (ESI, m/e) [M + 1]⁺ 550.4. |
| 19 | ¹H NMR (400 MHz, DMSO-d₆) δ ppm: 10.11 |
| | (s, 1H), 8.48 (d, J = 8.4 Hz, 1H), 7.58-7.29 (m, |
| | 4H), 7.13-7.05 (m, 2H), 6.73 (s, 1H), 4.37 (t, |
| | J = 8.8 Hz, 1H), 4.02 (s, 3H), 3.55-3.35 (m, 4H), |
| | 3.33-3.11 (m, 2H), 2.85 (t, J = 7.2 Hz, 1H), |
| | 2.58 (d, J = 4.4 Hz, 3H), 1.90-1.54 (m, 6H), |
| | 1.38-1.30 (m, 2H), 1.20-0.95 (m, 5H), 0.74 (t, |
| | J = 7.2 Hz, 3H). MS (ESI, m/e) [M + 1]⁺ 550.4. |
| 20 | ¹H NMR (400 MHz, DMSO-d₆) δ ppm: 10.11 |
| | (s, 1H), 8.48 (d, J = 8.4 Hz, 1H), 7.60-7.28 (m, |
| | 4H), 7.12 (d, J = 8.0 Hz, 1H), 7.06 (s, 1H), |
| | 6.62 (s, 1H), 4.37 (t, J = 8.4 Hz, 1H), 4.02 (s, |
| | 3H), 3.49-3.42 (m, 2H), 3.24-3.00 (m, 4H), |
| | 2.58 (d, J = 4.4 Hz, 3H), 1.90-1.57 (m, 6H), |
| | 1.20-1.10 (m, 5H), 1.07-0.95 (m, 3H). MS |
| | (ESI, m/e) [M + 1]⁺ 550.4. |
| 21 | ¹H NMR (400 MHz, DMSO-d₆) δ ppm: 10.12 |
| | (d, J = 3.2 Hz, 1H), 8.48 (d, J = 8.2 Hz, 1H), |
| | 7.46 (d, J = 1.6 Hz, 1H), 7.27-7.16 (m, 1H), |
| | 7.14-7.05 (m, 2H), 7.05-6.91 (m, 1H), 6.50 (d, |
| | J = 18.4 Hz, 1H), 4.96-4.83 (m, 1H), 4.42-4.32 |
| | (m, 1H), 4.03 (s, 3H), 3.64-3.45 (m, 2H), 3.30- |
| | 3.11 (m, 3H), 3.03-2.80 (m, 2H), 1.91-1.47 (m, |
| | 6H), 1.21-1.05 (m, 5H), 1.05-0.95 (m, 1H), |
| | 0.93-0.85 (m, 2H). MS (ESI, m/e) [M + 1]⁺. |
| | 495.3. |
| 22 | ¹H NMR (400 MHz, DMSO-d₆) δ ppm: 10.08 |
| | (s, 1H), 8.48 (d, J = 8.0 Hz, 1H), 7.57-7.30 (m, |
| | 3H), 7.14-7.05 (m, 2H), 6.37 (s, 1H), 4.99 (t, J = |
| | 5.2 Hz, 1H), 4.38 (t, J = 8.0 Hz, 1H), 4.02 (s, |
| | 3H), 3.63-3.40 (m, 3H), 3.33-3.05 (m, 6H), |
| | 1.90-1.55 (m, 6H), 1.21-1.10 (m, 5H), 1.07- |
| | 0.95 (m, 3H). MS (ESI, m/e) [M + 1]⁺ 509.3. |
| 23 | ¹H NMR (400 MHz, DMSO-d₆) δ ppm: 12.46 |
| | (br, 1H), 10.12 (s, 1H), 8.49 (d, J = 8.0 Hz, |
| | 1H), 7.57-7.30 (m, 3H), 7.14-7.05 (m, 2H), |
| | 6.61 (s, 1H), 4.38 (t, J = 8.0 Hz, 1H), 3.92 (s, |
| | 3H), 3.63-3.40 (m, 4H), 3.33-3.05 (m, 2H), |
| | 3.00-2.88 (m, 1H), 1.90-1.55 (m, 6H), 1.21- |
| | 1.10 (m, 5H), 1.07-0.95 (m, 3H). MS (ESI, |
| | m/e) [M + 1]⁺ 523.3. |
| 24 | ¹H NMR (400 MHz, DMSO-d₆) δ ppm: 10.11 |
| | (s, 1H), 8.48 (d, J = 8.0 Hz, 1H), 7.57-7.25 (m, |
| | 4H), 7.14-7.05 (m, 2H), 6.70 (s, 1H), 4.35 (t, J = |
| | 7.6 Hz, 1H), 4.02 (s, 3H), 3.63-3.40 (m, 3H), |
| | 3.33-3.05 (m, 3H), 2.75-2.68 (m, 1H), 2.58 (d, |
| | J = 4.4 Hz, 3H), 1.90-1.55 (m, 6H), 1.21-1.10 |
| | (m, 5H), 1.07-0.95 (m, 9H). MS (ESI, m/e) |
| | [M + 1]⁺ 578.4. |
| 25 | ¹H NMR (400 MHz, DMSO-d₆) δ ppm: 10.10 |
| | (s, 1H), 8.46 (d, J = 8.2 Hz, 1H), 7.53 (s, 2H), |
| | 7.46 (d, J = 2.0 Hz, 1H), 7.39-7.31 (m, 1H), |
| | 7.14-7.09 (m, 1H), 7.06 (s, 1H), 6.76-6.70 (m, |
| | 1H), 4.41-4.33 (m, 1H), 4.02 (s, 3H), 3.58-3.35 |
| | (s, 2H), 3.22-3.16 (m, 1H), 3.05-2.94 (m, 4H), |
| | 2.57 (d, J = 4.4 Hz, 3H), 1.86-1.77 (m, 2H), |
| | 1.31-0.93 (m, 20H). MS (ESI, m/e) [M + 1]⁺ |
| | 604.4. |
| 26 | ¹H NMR (400 MHz, DMSO-d₆) δ ppm: 10.11 |
| | (s, 1H), 8.47 (d, J = 8.2 Hz, 1H), 7.57-7.48 (m, |
| | 2H), 7.46 (d, J = 2.0 Hz, 1H), 7.42-7.30 (m, |
| | 1H), 7.15-7.08 (m, 1H), 7.08-7.04 (m, 1H), |
| | 6.71 (s, 1H), 4.42-4.30 (m, 1H), 4.02 (s, 3H), |
| | 3.54-3.35 (m, 5H), 3.29-3.09 (m, 2H), 2.90- |
| | 2.76 (m, 1H), 2.57 (d, J = 4.4 Hz, 3H), 2.03 - |
| | 1.52 (m, 7H), 1.35-1.27 (m, 2H), 1.20-1.07 (s, |
| | 5H), 0.84-0.77 (m, 3H). MS (ESI, m/e) |
| | [M + 1]⁺. 564.4. |
| 27 | ¹H NMR (400 MHz, DMSO-d₆) δ ppm: 10.09 |
| | (s, 1H), 8.48 (d, J = 8.4 Hz. 1H), 7.49-7.42 (m, |
| | 4H), 7.08-7.02 (m, 2H), 6.76 (s, 1H), 4.39-4.36 |
| | (m, 1H), 4.02 (s, 3H), 3.46-3.41 (m, 3H), 3.21- |
| | 3.01 (m, 2H), 2.54-2.51 (m, 3H), 2.24-2.21 (m, |
| | 1H),1.81-1.57 (m, 13H), 1.05-1.02 (m, 4H). |
| | MS (ESI, m/e) [M + 1]⁺ 576.4. |
| 28 | ¹H NMR (400 MHz, DMSO-d₆) δ ppm: 12.18 |
| | (br, 1H), 8.79 (s, 1H), 7.55-7.20 (m, 4H), 7.07 |
| | (s, 1H), 6.73 (s, 1H), 5.02 (t, J = 5.6 Hz, 1H), |
| | 4.02 (s, 3H), 3.64-3.40 (m, 4H), 3.32-3.14 (m, |
| | 2H), 2.97-2.89 (m, 1H), 2.58 (d, J = 4.4 Hz, |
| | 3H), 2.15-1.95 (m, 2H), 1.90-1.35 (m, 6H), |
| | 1.21-0.95 (m, 5H), 0.71 (d, J = 6.8 Hz, 3H), |
| | 0.66 (d, J = 6.8 Hz, 3H). MS (ESI, m/e) |
| | [M + 1]⁺ 561.3. |
| 29 | ¹H NMR (400 MHz, DMSO-d₆) δ ppm: 12.18 |
| | (br, 1H), 9.41 (d, J = 9.2 Hz, 1H), 7.59-7.20 |
| | (m, 3H), 6.73 (s, 1H), 5.06 (t, J = 8.0 Hz, 1H), |
| | 3.67-3.40 (m, 4H), 3.32-3.14 (m, 2H), 2.97- |
| | 2.89 (m, 1H), 2.58 (d, J = 4.4 Hz, 3H), 2.11- |
| | 1.95 (m, 2H), 1.90-1.39 (m, 6H), 1.21-0.95 (m, |
| | 4H), 0.71 (d, J = 6.8 Hz, 3H), 0.66 (d, J = 6.8 |
| | Hz, 3H). MS (ESI, m/e) [M + 1]⁺ 563.3. |
| 30 | ¹H NMR (400 MHz, DMSO-d₆) δ ppm: 10.14 |
| | (s, 1H), 9.09 (d, J = 8.0 Hz, 1H), 7.59-7.29 (m, |
| | 3H), 7.13 (d, J = 8.4 Hz, 1H), 6.78 (s, 1H), |
| | 4.45 (t, J = 8.0 Hz, 1H), 3.59-3.46 (m, 2H), |
| | 3.32-3.10 (m, 4H), 2.93-2.87 (m, 1H), 2.58 (d, |
| | J = 4.4 Hz, 3H), 1.90-1.39 (m, 7H), 1.21-0.95 |
| | (m, 5H), 0.75 (d, J = 6.8 Hz, 3H), 0.70 (d, J = |
| | 6.4 Hz, 3H). MS (ESI, m/e) [M + 1]⁺ 566.3. |
| 31 | ¹H NMR (400 MHz, DMSO-d₆) δ ppm: 12.20 |
| | (br, 1H), 8.78 (d, J = 8.0 Hz, 1H), 7.55-7.20 |
| | (m, 4H), 7.07 (s, 1H), 6.69 (s, 1H), 5.01 (t, J = |
| | 8.4 Hz, 1H), 4.02 (s, 3H), 3.65-3.40 (m, 4H), |
| | 3.32-3.23 (m, 2H), 2.93-2.87 (m, 1H), 2.58 (d, |
| | J = 4.4 Hz, 3H), 2.10-1.55 (m, 5H), 1.42-1.27 |
| | (m, 3H), 1.21-0.95 (m, 5H), 0.70 (t, J = 7.2 Hz, |
| | 3H). MS (ESI, m/e) [M + 1]⁺ 547.3. |
| 33 | ¹H NMR (400 MHz, DMSO-d₆) δ ppm: 12.28 |
| | (br, 1H), 9.42 (d, J = 8.8 Hz, 1H), 7.55-7.50 |
| | (m, 1H), 7.32 (s, 2H), 6.69 (s, 1H), 5.06 (t, J = |
| | 8.8 Hz, 1H), 3.62-3.40 (m, 4H), 3.32-3.23 (m, |
| | 2H), 2.93-2.87 (m, 1H), 2.58 (d, J = 4.4 Hz, |
| | 3H), 2.14-1.55 (m, 5H), 1.42-1.27 (m, 3H), |
| | 1.21-0.95 (m, 5H), 0.71 (t, J = 7.2 Hz, 3H). |
| | MS (ESI, m/e) [M + 1]⁺ 549.4. |
| 34 | ¹H NMR (400 MHz, DMSO-d₆) δ ppm: 10.09 |
| | (s, 1H), 8.53 (d, J = 8.4 Hz. 1H), 7.49-7.42 (m, |
| | 4H), 7.08-7.02 (m, 2H), 6.76 (s, 1H), 4.39-4.36 |
| | (m, 1H), 4.02 (s, 3H), 3.46-3.41 (m, 6H), 3.21- |
| | 3.01 (m, 4H), 2.54-2.51 (m, 3H), 1.81-1.57 (m, |
| | 7H), 1.31-1.17 (m, 5H), 1.05-1.02 (m, 2H). |
| | MS (ESI, m/e) [M + 1]⁺ 592.3. |
| 35 | ¹H NMR (400 MHz, DMSO-d₆) δ ppm: 10.09 |
| | (s, 1H), 8.53 (d, J = 8.4 Hz. 1H), 7.49-7.42 (m, |
| | 4H), 7.08-7.02 (m, 2H), 6.76 (s, 1H), 4.39-4.36 |
| | (m, 1H), 4.02 (s, 3H), 3.46-3.41 (m, 3H), 3.21- |
| | 3.01 (m, 10H), 2.54-2.51 (m, 3H), 1.81-1.57 |
| | (m, 7H), 1.31-1.17 (m, 6H), 1.05-1.02 (m, 2H), |
| | 0.63-0.55 (m, 2H). MS (ESI, m/e) [M + 1]⁺ |
| | 594.3. |
| 36 | MS (ESI, m/e) [M + 1]⁺ 598.3. |
| 37 | ¹H NMR (400 MHz, DMSO-d₆) δ ppm: 10.15 |
| | (d, J = 20.0 Hz, 1H), 8.52-8.47 (m, 1H), 7.59- |
| | 7.45 (m, 2H), 7.41-7.34 (m, 1H), 7.25-7.12 (m, |
| | 4H), 7.09-7.02 (m, 3H), 6.93 (d, J = 8.0 Hz, |
| | 1H), 6.74 (d, J = 4.0 Hz, 1H), 4.42-4.36 (m, |
| | 1H), 4.02 (d, J = 8.0 Hz, 1H), 3.69-3.63 (m, |
| | 1H), 3.50-3.41 (m, 2H), 3.31-3.27 (m, 2H), |
| | 3.12-3.05 (m, 2H), 2.98-2.95 (m, 1H), 2.72- |
| | 2.65 (m, 1H), 2.55-2.53 (m, 3H), 1.90-1.79 (m, |
| | 2H), 1.75-1.54 (m, 4H), 1.35-1.10 (m, 8H), |
| | 1.02-0.78 (m, 2H). MS (ESI, m/e) [M + 1]⁺. |
| 38 | ¹H NMR (400 MHz, DMSO-d₆) δ ppm: 10.11 |
| | (s, 1H), 8.49 (d, J = 8.0 Hz, 1H), 7.57-7.29 (m, |
| | 4H), 7.12 (d, J = 8.4 Hz, 1H), 7.06 (s, 1H), |
| | 6.77 (s, 1H), 4.37 (t, J = 8.8 Hz, 1H), 4.02 (s, |
| | 3H), 3.58-3.45 (m, 2H), 3.33-3.11 (m, 4H), |
| | 2.94-2.87 (m, 1H), 2.58 (d, J = 4.4 Hz, 3H), |
| | 1.90-1.54 (m, 6H), 1.48-1.40 (m, 2H), 1.20- |
| | 0.95 (m, 5H),), 0.75 (d, J = 6.4 Hz, 3H), 0.70 |
| | (d, J = 6.8 Hz, 3H). MS (ESI, m/e) [M + 1]⁺ |
| | 564.3. |
| 39 | ¹H NMR (400 MHz, DMSO-d₆) δ ppm: 10.11 |
| | (s, 1H), 8.49 (d, J = 8.0 Hz, 1H), 7.59-7.29 (m, |
| | 4H), 7.12 (d, J = 8.0 Hz, 1H), 7.06 (d, J = 2.0 |
| | Hz, 1H), 6.77 (s, 1H), 4.37 (t, J = 8.4 Hz, 1H), |
| | 4.02 (s, 3H), 3.57-3.45 (m, 2H), 3.32-3.10 (m, |
| | 4H), 2.93-2.87 (m, 1H), 2.58 (d, J = 4.4 Hz, |
| | 3H), 1.90-1.39 (m, 7H), 1.21-0.95 (m, 5H), |
| | 0.74 (d, J = 6.8 Hz, 3H), 0.70 (d, J = 6.4 Hz, |
| | 3H). MS (ESI, m/e) [M + 1]⁺ 564.3. |
| 40 | ¹H NMR (400 MHz, DMSO-d₆) δ ppm: 10.15 |
| | (s, 1H), 9.09 (d, J = 8.0 Hz, 1H), 7.55-7.27 (m, |
| | 3H), 7.13 (d, J = 8.4 Hz, 1H), 6.78 (s, 1H), |
| | 4.46 (t, J = 8.0 Hz, 1H), 3.57-3.45 (m, 2H), |
| | 3.32-3.10 (m, 4H), 2.93-2.87 (m, 1H), 2.58 (d, |
| | J = 4.4 Hz, 3H), 1.90-1.39 (m, 7H), 1.21-1.00 |
| | (m, 5H), 0.77-0.50 (m, 6H). MS (ESI, m/e) |
| | [M + 1]⁺ 566.5. |
| 41 | ¹H NMR (400 MHz, DMSO-d₆) δ ppm: 10.12 |
| | (s, 1H), 8.49 (d, J = 8.4 Hz, 1H), 7.58-7.27 (m, |
| | 4H), 7.15-7.10 (m, 1H), 7.06 (d, J = 1.6 Hz, |
| | 1H), 6.73 (s, 1H), 4.37 (t, J = 8.0 Hz, 1H), 4.02 |
| | (s, 3H), 3.56-3.35 (m, 3H), 3.30-3.10 (m, 2H), |
| | 3.04-2.89 (m, 2H), 2.58 (d, J = 4.4 Hz, 3H), |
| | 1.90-1.55 (m, 6H), 1.20-0.78 (m, 5H), 0.38- |
| | 0.28 (m, 2H), 0.15-0.09 (m, 2H). MS (ESI, |
| | m/e) [M + 1]⁺ 562.3. |
| 42 | ¹H NMR (400 MHz, DMSO-d₆) δ ppm: 10.11 |
| | (s, 1H), 8.53 (d, J = 8.4 Hz. 1H), 7.49-7.22 (m, |
| | 4H), 7.08-7.02 (m, 2H), 6.76 (s, 1H), 4.59-4.56 |
| | (m, 1H), 4.39-4.35 (m, 1H), 4.03 (s, 3H), 3.46- |
| | 3.41 (m, 3H), 3.21-3.01 (m, 2H), 2.54-2.51 (m, |
| | 3H), 1.91-1.57 (m, 7H), 1.31-1.17 (m, 2H), |
| | 1.05-1.02 (m, 2H), 0.87-0.81 (m, 7H). MS |
| | (ESI, m/e) [M + 1]⁺ 580.3. |
| 43 | MS (ESI, m/e) [M + 1]⁺ 613.4. |
| 44 | ¹H NMR (400 MHz, DMSO-d₆) δ ppm: 10.11 |
| | (d, J = 4.0 Hz, 1H), 8.48 (d, J = 8.0 Hz, 1H), |
| | 7.56-7.47 (m, 2H), 7.46 (d, J = 4.0 Hz, 1H), |
| | 7.42-7.30 (m, 1H), 7.12-7.10 (m, 1H), 7.06 (d, |
| | J = 4.0 Hz, 1H), 6.79 (s, 1H), 4.40-4.35 (m, |
| | 1H), 4.02 (s, 3H), 3.60-3.50 (m, 1H), 3.45-3.41 |
| | (m, 1H), 3.30-3.28 (m, 1H), 3.24-3.10 (m, 2H), |
| | 2.99-2.94 (m, 1H), 2.57 (d, J = 4.0 Hz, 3H), |
| | 1.83-1.81 (m, 2H), 1.73-1.68 (m, 2H), 1.62- |
| | 1.53 (m, 2H), 1.23-0.79 (m, 6H), 0.66 (s, 9H). |
| | MS (ESI, m/e) [M + 1]⁺ 578.4. |
| 45 | ¹H NMR (400 MHz, DMSO-d₆) δ ppm: 10.06 |
| | (s, 1H), 8.50 (d, J = 7.6 Hz, 1H), 7.58-7.27 (m, |
| | 4H), 7.12 (d, J = 8.0 Hz, 1H), 7.03 (d, J = 1.6 |
| | Hz, 1H), 6.77 (s, 1H), 4.54 (t, J = 9.6 Hz, 1H), |
| | 4.03 (s, 3H), 3.57-3.45 (m, 2H), 3.32-3.10 (m, |
| | 4H), 2.93-2.87 (m, 1H), 2.80-2.67 (m, 1H), |
| | 2.58 (d, J = 4.4 Hz, 3H), 2.10-1.75 (m, 1H), |
| | 1.50-1.40 (m, 1H), 0.72-0.66 (m, 6H). MS |
| | (ESI, m/e) [M + 1]⁺ 536.5. |
| 46 | ¹H NMR (400 MHz, DMSO-d₆) δ ppm: 10.10 |
| | (s, 1H), 8.61 (d, J = 8.0 Hz, 1H), 7.58-7.30 (m, |
| | 4H), 7.12 (d, J = 8.0 Hz, 1H), 7.05 (d, J = 1.6 |
| | Hz, 1H), 6.77 (s, 1H), 4.37 (t, J = 9.2 Hz, 1H), |
| | 4.03 (s, 3H), 3.57-3.45 (m, 2H), 3.32-3.12 (m, |
| | 4H), 2.93-2.87 (m, 1H), 2.58 (d, J = 4.4 Hz, |
| | 3H), 2.40-2.30 (m, 1H), 1.89-1.80 (m, 1H), |
| | 1.70-1.28 (m, 9H), 0.75 (d, J = 6.4 Hz, 3H), |
| | 0.70 (d, J = 6.8 Hz, 3H). MS (ESI, m/e) |
| | [M + 1]⁺ 550.5. |
| 47 | ¹H NMR (400 MHz, DMSO-d₆) δ ppm: 10.11 |
| | (s, 1H), 8.50 (d, J = 8.8 Hz, 1H), 7.57-7.30 (m, |
| | 4H), 7.12 (d, J = 8.0 Hz, 1H), 7.06 (d, J = 1.6 |
| | Hz, 1H), 6.77 (s, 1H), 4.45 (t, J = 8.0 Hz, 1H), |
| | 4.03 (s, 3H), 3.57-3.45 (m, 2H), 3.32-3.12 (m, |
| | 4H), 2.93-2.87 (m, 1H), 2.58 (d, J = 4.4 Hz, |
| | 3H), 2.13-2.04 (m, 1H), 1.78-1.28 (m, 14H), |
| | 0.74 (d, J = 6.4 Hz, 3H), 0.70 (d, J = 6.4 Hz, |
| | 3H). MS (ESI, m/e) [M + 1]⁺ 578.4. |
| 48 | ¹H NMR (400 MHz, DMSO-d₆) δ ppm: 10.22- |
| | 10.19 (m, 1H), 8.58-8.54 (m, 1H), 7.73-7.56 |
| | (m, 1H), 7.54-7.50 (m, 1H), 7.47-7.40 (m, 1H), |
| | 7.32-7.24 (m, 3H), 7.22-7.10 (m, 4H), 7.08- |
| | 7.02 (m, 1H), 6.86-6.72 (m, 1H), 4.47-4.43 (m, |
| | 1H), 4.10-4.07 (m, 3H), 3.17-3.02 (m, 3H), |
| | 2.89-2.76 (m, 1H), 2.61-2.59 (m, 2H), 2.09- |
| | 2.02 (m, 1H), 1.95-1.86 (m, 2H), 1.80-1.73 (m, |
| | 2H), 1.71-1.62 (m, 2H), 1.42 (s, 1H), 1.30-1.20 |
| | (m, 9H), 1.14-1.05 (m, 2H). MS (ESI, m/e) |
| | [M + 1]⁺ 626.4. |
| 49 | ¹H NMR (400 MHz, DMSO-d₆) δ ppm: 10.00 |
| | (s, 1H), 8.27 (d, J = 8.4 Hz, 1H), 7.55-7.25 (m, |
| | 3H), 7.11 (d, J = 8.4 Hz, 1H), 6.76 (s, 1H), |
| | 4.29 (t, J = 8.0 Hz, 1H), 3.59-3.45 (m, 2H), |
| | 3.32-3.10 (m, 4H), 2.94-2.87 (m, 1H), 2.58 (d, |
| | J = 4.4 Hz, 3H), 1.81-1.40 (m, 7H), 1.21-0.80 |
| | (m, 7H), 0.74 (d, J = 6.8 Hz, 3H), 0.72-0.66 |
| | (m, 6H). MS (ESI, m/e) [M + 1]⁺ 524.4. |
| 50 | ¹H NMR (400 MHz, DMSO-d₆) δ ppm: 9.94 |
| | (s, 1H), 7.55-7.20 (m, 4H), 7.11 (d, J = 8.0 Hz, |
| | 1H), 6.77 (s, 1H), 4.02-3.93 (m, 3H), 3.59-3.45 |
| | (m, 2H), 3.32-3.10 (m, 4H), 2.94-2.87 (m, 1H), |
| | 2.58 (d, J = 4.8 Hz, 3H), 1.78-1.40 (m, 7H), |
| | 1.21-0.93 (m, 8H), 0.74 (d, J = 6.8 Hz, 3H), |
| | 0.69 (d, J = 6.4 Hz, 3H). MS (ESI, m/e) |
| | [M + 1]⁺ 528.3. |
| 51 | ¹H NMR (400 MHz, DMSO-d₆) δ ppm: 10.03 |
| | (s, 1H), 8.01 (d, J = 8.4 Hz, 1H), 7.58-7.08 (m, |
| | 9H), 6.77 (s, 1H), 4.03 (t, J = 8.4 Hz, 1H), |
| | 3.59-3.45 (m, 2H), 3.32-3.10 (m, 4H), 2.94- |
| | 2.87 (m, 1H), 2.58 (d, J = 4.8 Hz, 3H), 1.85- |
| | 1.40 (m, 7H), 1.21-1.03 (m, 5H), 0.77-0.66 (m, |
| | 6H). MS (ESI, m/e) [M + 1]⁺ 576.4. |
| 52 | ¹H NMR (400 MHz, DMSO-d₆) δ ppm: 9.98 |
| | (s, 1H), 8.02 (d, J = 8.8 Hz, 1H), 7.55-7.08 (m, |
| | 9H), 6.76 (s, 1H), 4.27 (t, J = 8.0 Hz, 1H), |
| | 3.59-3.45 (m, 2H), 3.32-3.10 (m, 4H), 2.94- |
| | 2.87 (m, 1H), 2.84-2.78 (m, 2H), 2.58 (d, J = |
| | 4.8 Hz, 3H), 2.48-2.38 (m, 2H), 1.70-1.40 (m, |
| | 7H), 1.21-0.83 (m, 5H), 0.74 (d, J = 6.8 Hz, |
| | 3H), 0.69 (d, J = 6.4 Hz, 3H). MS (ESI, m/e) |
| | [M + 1]⁺ 588.4. |
| 53 | ¹H NMR (400 MHz, DMSO-d₆) δ ppm: 9.99 |
| | (s, 1H), 8.26 (d, J = 8.4 Hz, 1H), 7.55-7.17 (m, |
| | 8H), 7.11 (d, J = 8.4 Hz, 1H), 6.76 (s, 1H), |
| | 4.28 (t, J = 8.0 Hz, 1H), 3.59-3.45 (m, 4H), |
| | 3.32-3.10 (m, 4H), 2.94-2.86 (m, 1H), 2.58 (d, |
| | J = 4.8 Hz, 3H), 1.75-1.40 (m, 7H), 1.21-0.95 |
| | (m, 5H), 0.77-0.66 (m, 6H). MS (ESI, m/e) |
| | [M + 1]⁺ 574.4. |
| 54 | ¹H NMR (400 MHz, DMSO-d₆) δ ppm: 10.08 |
| | (s, 1H), 8.46 (d, J = 8.0 Hz, 1H), 7.91 (d, J = |
| | 7.2 Hz, 1H), 7.58-7.29 (m, 6H), 7.12 (d, J = |
| | 8.4 Hz, 1H), 6.76 (s, 1H), 4.41 (t, J = 8.4 Hz, |
| | 1H), 3.59-3.45 (m, 2H), 3.32-3.10 (m, 4H), |
| | 2.90 (t, J = 8.0 Hz, 1H), 2.58 (d, J = 4.4 Hz, |
| | 3H), 1.93-1.55 (m, 6H), 1.48-1.38 (m, 1H), |
| | 1.21-0.95 (m, 5H), 0.76-0.66 (m, 6H). MS |
| | (ESI, m/e) [M + 1]⁺ 560.3. |
| 55 | ¹H NMR (400 MHz, DMSO-d₆) δ ppm: 10.11 |
| | (s, 1H), 8.48 (d, J = 8.4 Hz, 1H), 7.77-7.72 (m, |
| | 1H), 7.54-7.33 (m, 4H), 7.11 (d, J = 8.0 Hz, |
| | 1H), 7.06 (d, J = 2.0 Hz, 1H), 4.37 (t, J = 8.8 |
| | Hz, 1H), 4.02 (s, 3H), 3.55-3.40 (m, 5H), 3.26- |
| | 3.10 (m, 2H), 2.60 (d, J = 4.4 Hz, 3H), 2.04- |
| | 1.56 (m, 7H), 1.20-0.95 (m, 5H), 0.91 (d, J = |
| | 6.4 Hz, 3H), 0.82 (d, J = 6.8 Hz, 3H). MS |
| | (ESI, m/e) [M + 1]⁺ 600.3. |
| 56 | ¹H NMR (400 MHz, DMSO-d₆) δ ppm: 10.16- |
| | 10.12 (m, 1H), 8.48 (d, J = 8.0 Hz, 1H), 7.57- |
| | 7.51 (m, 1H), 7.47-7.45 (m, 1H), 7.41-7.33 (m, |
| | 1H), 7.16-7.09 (m, 2H), 7.07-7.05 (m, 1H), |
| | 6.95-6.88 (m, 2H), 6.81-6.76 (m, 2H), 6.70 (s, |
| | 1H), 4.42-4.36 (m, 1H), 4.02 (d, J = 4.0 Hz, |
| | 3H), 3.70-3.68 (m, 2H), 3.64 (s, 1H), 3.47-3.38 |
| | (m, 2H), 3.29-3.25 (m, 1H), 3.12-3.06 (m, 2H), |
| | 2.99-2.95 (m, 1H), 2.66-2.59 (m, 2H), 2.54- |
| | 2.52 (m, 3H), 2.45-2.34 (m, 1H), 1.89-1.81 (m, |
| | 2H), 1.73-1.56 (m, 4H), 1.23-1.15 (m, 6H), |
| | 1.04-0.99 (m, 1H). MS (ESI, m/e) [M + 1]⁺ |
| | 642.5. |
| 57 | ¹H NMR (400 MHz, DMSO-d₆) δ ppm: 10.15- |
| | 10.12 (m, 1H), 8.50 (d, J = 8.0 Hz, 1H), 7.55- |
| | 7.45 (m, 2H), 7.39-7.24 (m, 3H), 7.11-7.07 (m, |
| | 2H), 6.93-6.90 (m, 1H), 6.78-6.69 (m, 2H), |
| | 4.40-4.35 (m, 1H), 4.02 (d, J = 4.0 Hz, 3H), |
| | 3.74-3.64 (m, 1H), 3.45-3.41 (m, 1H), 3.13- |
| | 3.00 (m, 3H), 2.89-2.79 (m, 2H), 2.56-2.54 (m, |
| | 3H), 2.03-1.95 (m, 1H), 1.87-1.82 (m, 1H), |
| | 1.73-1.57 (m, 4H), 1.23-1.11 (m, 8H). MS |
| | (ESI, m/e) [M + 1]⁺ 618.4. |
| 58 | ¹H NMR (400 MHz, DMSO-d₆) δ ppm: 10.13 |
| | (s, 1H), 9.04 (s, 1H), 8.88-8.73 (m, 1H), 8.71 |
| | (d, J = 4.8 Hz, 1H), 8.25 (d, J = 8.0 Hz, 1H), |
| | 7.57-7.29 (m, 4H), 7.12 (d, J = 8.4 Hz, 1H), |
| | 6.76 (s, 1H), 4.47-4.40 (m, 1H), 3.59-3.45 (m, |
| | 2H), 3.32-3.10 (m, 4H), 2.94-2.87 (m, 1H), |
| | 2.58 (d, J = 4.4 Hz, 3H), 1.94-1.40 (m, 7H), |
| | 1.21-0.95 (m, 5H), 0.74 (d, J = 6.4 Hz, 3H), |
| | 0.70 (d, J = 6.8 Hz, 3H). MS (ESI, m/e) |
| | [M + 1]⁺ 561.4. |
| 59 | ¹H NMR (400 MHz, DMSO-d₆) δ ppm: 10.10 |
| | (s, 1H), 8.48 (d, J = 8.4 Hz, 1H), 7.57-7.30 (m, |
| | 4H), 7.12 (d, J = 8.4 Hz, 1H), 6.95 (d, J = 1.0 |
| | Hz, 1H), 6.77 (s, 1H), 5.45-5.35 (m, 1H), 4.38- |
| | 4.32 (m, 1H), 3.59-3.45 (m, 2H), 3.30-3.12 (m, |
| | 4H), 2.93-2.87 (m, 1H), 2.58 (d, J = 4.4 Hz, |
| | 3H), 1.90-1.40 (m, 7H), 1.35 (t, J = 7.2 Hz, |
| | 6H), 1.20-0.95 (m, 5H), 0.75 (d, J = 6.8 Hz, |
| | 3H), 0.70 (d, J = 6.4 Hz, 3H). MS (ESI, m/e) |
| | [M + 1]⁺ 592.5. |
| 60 | ¹H NMR (400 MHz, DMSO-d₆) δ ppm: 10.32 |
| | (s, 1H), 9.01 (d, J = 7.6 Hz, 1H), 7.57-7.30 (m, |
| | 9H), 7.13 (s, 1H), 7.12 (d, J = 7.6 Hz, 1H), |
| | 6.76 (s, 1H), 5.80 (d, J = 7.6 Hz, 1H), 4.04 (s, |
| | 3H), 3.59-3.44 (m, 2H), 3.30-3.12 (m, 4H), |
| | 2.93-2.87 (m, 1H), 2.57 (d, J = 4.4 Hz, 3H), |
| | 1.48-1.38 (m, 1H), 0.76-0.66 (m, 6H). MS |
| | (ESI, m/e) [M + 1]⁺ 558.3. |
| 61 | ¹H NMR (400 MHz, DMSO-d₆) δ ppm: 10.07 |
| | (s, 1H), 8.24 (s, 1H), 8.10-8.05 (m, 3H), 7.93 |
| | (s, 1H), 7.57-7.30 (m, 3H), 7.11 (d, J = 8.0 Hz, |
| | 1H), 6.76 (s, 1H), 4.43-4.35 (m, 1H), 3.85 (s, |
| | 3H), 3.59-3.45 (m, 2H), 3.32-3.10 (m, 4H), |
| | 2.94-2.87 (m, 1H), 2.58 (d, J = 4.4 Hz, 3H), |
| | 1.85-1.40 (m, 7H), 1.21-0.95 (m, 5H), 0.74 (d, |
| | J = 6.4 Hz, 3H), 0.69 (d, J = 6.4 Hz, 3H). MS |
| | (ESI, m/e) [M + 1]⁺ 564.4. |
| 62 | ¹H NMR (400 MHz, DMSO-d₆) δ ppm: 10.17- |
| | 10.12 (m, 1H), 8.51-8.47 (m, 1H), 7.58-7.46 |
| | (m, 2H), 7.41-7.34 (m, 1H), 7.20-6.99 (m, 5H), |
| | 6.91-6.89 (m, 1H), 6.85-6.81 (m, 1H), 6.70 (s, |
| | 1H), 4.43-4.36 (m, 1H), 4.02-4.01 (m, 3H), |
| | 3.65-3.59 (m, 1H), 3.46-3.40 (m, 2H), 3.31- |
| | 3.26 (m, 2H), 3.13-3.04 (m, 2H), 2.98-2.92 (m, |
| | 1H), 2.68-2.60 (m, 1H), 2.55-2.53 (m, 3H), |
| | 2.47-2.31 (m, 1H), 2.23-2.15 (m, 3H), 1.89- |
| | 1.82 (m, 1H), 1.75-1.54 (m, 3H), 1.23-1.14 (m, |
| | 6H). MS (ESI, m/e) [M + 1]⁺ 626.4. |
| 63 | ¹H NMR (400 MHz, DMSO- d6) δ ppm: 10.10 |
| | (s, 1H), 8.46 (d, J = 8.6 Hz, 1H), 7.58-7.47 (m, |
| | 2H), 7.46 (d, J = 1.8 Hz, 1H) 7.41-7.28 (m, |
| | 1H), 7.11 (d, J = 7.8 Hz, 1H), 7.07-7.03 (m, |
| | 1H), 6.74 (s, 1H), 4.42-4.31 (m, 1H), 4.02 (s, |
| | 3H), 3.65-3.54 (m, 1H), 3.52-3.37 (m, 3H), |
| | 3.23-3.05 (m, 3H), 2.84-2.78 (m, 1H), 2.58 (d, |
| | J = 4.4 Hz, 3H), 2.39-2.22 (m, 2H), 2.03-1.50 |
| | (m, 7H), 1.21-1.08 (m, 4H). MS (ESI, m/e) |
| | [M + 1]⁺ 560.4. |
| 64 | ¹H NMR (400 MHz, DMSO-d₆) δ ppm: 10.15 |
| | (s, 1H), 8.38 (d, J = 7.2 Hz, 1H), 8.24 (s, 1H), |
| | 7.55-7.27 (m, 3H), 7.13 (d, J = 8.4 Hz, 1H), |
| | 6.76 (s, 1H), 4.52-4.45 (m, 1H), 4.13 (s, 3H), |
| | 3.58-3.45 (m, 2H), 3.32-3.12 (m, 4H), 2.94- |
| | 2.87 (m, 1H), 2.58 (d, J = 4.4 Hz, 3H), 1.90- |
| | 1.40 (m, 7H), 1.21-0.95 (m, 5H), 0.76 (d, J = |
| | 6.4 Hz, 3H), 0.71 (d, J = 6.4 Hz, 3H). MS |
| | (ESI, m/e) [M + 1]⁺ 565.5. |
| 65 | ¹H NMR (400 MHz, DMSO-d₆) δ ppm: 10.09 |
| | (s, 1H), 8.47-8.44 (m, 1H), 7.53-7.46 (m, 3H), |
| | 7.21-7.06 (m, 3H), 6.68-6.65 (m, 2H), 4.40- |
| | 4.35 (m, 1H), 4.02 (s, 3H), 3.54-3.38 (m, 3H), |
| | 3.31-3.29 (m, 2H), 2.98-2.91 (m, 2H), 2.68- |
| | 2.65 (m, 1H), 2.59-2.56 (m, 3H), 2.34-2.31 (m, |
| | 1H), 2.02-1.95 (m, 6H), 1.85-1.81 (m, 1H), |
| | 1.75-1.67 (m, 2H), 1.48-1.44 (m, 2H), 1.23- |
| | 1.14 (m, 5H). MS (ESI, m/e) [M + 1]⁺ 576.5. |
| 66 | ¹H NMR (400 MHz, DMSO-d₆) δ ppm: 10.15 |
| | (s, 1H), 8.74 (d, J = 8.0 Hz, 1H), 7.54-7.25 (m, |
| | 8H), 7.20-7.12 (m, 2H), 6.95 (s, 1H), 6.77 (s, |
| | 1H), 4.83-4.77 (m, 1H), 3.94 (s, 3H), 3.59-3.34 |
| | (m, 2H), 3.31 (s, 1H), 3.24-3.12 (m, 3H), 3.07- |
| | 3.01 (m, 1H), 2.92-2.88 (m, 1H), 2.57 (d, J = |
| | 4.0 Hz, 1H), 1.48-1.40 (m, 1H), 1.23-1.16 (m, |
| | 1H), 0.76-0.69 (m, 6H). MS (ESI, m/e) [M + 1]⁺ |
| | 572.3. |
| 67 | ¹H NMR (400 MHz, DMSO-d₆) δ ppm: 10.18 |
| | (s, 1H), 8.62 (d, J = 12.0 Hz, 1H), 7.56-7.46 |
| | (m, 3H), 7.41-7.30 (m, 1H), 7.12 (d, J = 8.0 |
| | Hz, 1H), 7.06 (d, J = 4.0 Hz, 1H), 6.76 (s, 1H), |
| | 4.49-4.45 (m, 1H), 4.03 (s, 3H), 3.57-3.45 (m, |
| | 2H), 3.30 (s, 1H), 3.23-3.15 (m, 2H), 3.12-3.05 |
| | (m, 2H), 2.93-2.88 (m, 1H), 2.57 (d, J = 8.0 |
| | Hz, 1H), 2.07-1.99 (m, 3H), 1.92-1.65 (m, |
| | 4H), 1.46-1.42 (m, 2H), 1.35-1.23 (m, 1H), |
| | 0.75-0.68 (m, 6H). MS (ESI, m/e) [M + 1]⁺ |
| | 600.4. |
| 68 | ¹H NMR (400 MHz, DMSO-d₆) δ ppm: 10.11 |
| | (s, 1H), 8.48 (d, J = 8.0 Hz, 1H), 8.23-8.17 (m, |
| | 1H), 7.57-7.32 (m, 3H), 7.13 (d, J = 8.0 Hz, |
| | 1H), 7.06 (s, 1H), 6.90 (s, 1H), 4.42-4.35 (m, |
| | 1H), 4.02 (s, 3H), 3.95-3.74 (m, 2H), 3.64-3.38 |
| | (m, 3H), 3.32-3.07 (m, 3H), 2.87-2.80 (m, 1H), |
| | 1.90-1.40 (m, 7H), 1.21-0.95 (m, 5H), 0.76 (d, |
| | J = 6.4 Hz, 3H), 0.71 (d, J = 6.4 Hz, 3H). MS |
| | (ESI, m/e) [M + 1]⁺ 632.5. |
| 69 | ¹H NMR (400 MHz, DMSO-d₆) δ ppm: 10.09 |
| | (s, 1H), 8.63 (d, J = 8.2 Hz, 1H), 8.00-7.94 (m, |
| | 1H), 7.86 (d, J = 7.8 Hz, 1H), 7.64-7.46 (m, |
| | 4H), 7.42-7.28 (m, 1H), 7.11 (d, J = 8.2 Hz, |
| | 1H), 6.74 (s, 1H), 4.47-4.35 (m, 1H), 3.60-3.43 |
| | (m, 2H), 3.31-3.05 (m, 4H), 2.95-2.85 (m, 1H), |
| | 2.57 (d, J = 4.4 Hz, 3H), 1.94-1.53 (m, 6H), |
| | 1.50-1.38 (m, 1H), 1.20-1.01 (m, 5H), 0.78- |
| | 0.62 (m, 6H). MS (ESI, m/e) [M + 1]⁺ 594.4. |
| 70 | ¹H NMR (400 MHz, DMSO-d₆) δ ppm: 10.03 |
| | (s, 1H), 8.43 (d, J = 8.4 Hz. 1H), 7.49-7.42 (m, |
| | 4H), 7.08-7.02 (m, 2H), 6.36 (s, 1H), 4.39-4.36 |
| | (m, 1H), 4.02 (s, 3H), 3.46-3.41 (m, 2H), 3.07- |
| | 3.01 (m, 4H), 2.98-2.96 (m, 2H), 2.54-2.51 (m, |
| | 3H).1.81-1.67 (m, 7H), 1.05-1.02 (m, 6H), |
| | 0.79-0.72 (m, 3H). MS (ESI, m/e) [M + 1]⁺ |
| | 564.5. |
| 71 | ¹H NMR (400 MHz, DMSO-d₆) δ ppm: 10.07 |
| | (s, 1H), 8.33 (d, J = 8.4 Hz, 1H), 7.73-7.62 (m, |
| | 2H), 7.53-7.32 (m, 5H), 7.10-7.05 (m, 1H), |
| | 6.75 (s, 1H), 4.45-4.41 (m, 1H), 3.46-3.41 (m, |
| | 2H), 3.22-3.16 (m, 2H), 2.98-2.96 (m, 1H), |
| | 2.64-2.60 (m, 3H), 2.31 (s, 3H), 1.81-1.67 (m, |
| | 7H), 1.21-1.02 (m, 5H), 0.65-0.55 (m, 6H). |
| | MS (ESI, m/e) [M + 1]⁺ 574.5. |
| 72 | ¹H NMR (400 MHz, DMSO-d₆) δ ppm: 10.11 |
| | (s, 1H), 8.49 (d, J = 8.2 Hz, 1H), 7.60-7.44 (m, |
| | 3H), 7.44-7.26 (m, 1H), 7.11 (d, J = 8.2 Hz, |
| | 1H), 7.07-7.03 (m, 1H), 6.76 (s, 1H), 4.43-4.31 |
| | (m, 1H), 4.02 (s, 3H), 3.53-3.44 (m, 2H), 3.23- |
| | 3.15 (m, 5H), 3.12-3.04 (m, 2H), 2.94-2.88 (m, |
| | 1H), 2.57 (d, J = 4.6 Hz, 3H), 2.08-1.75 (m, |
| | 4H), 1.65-1.52 (m, 1H), 1.50-1.28 (m, 4H), |
| | 1.20-1.14 (m, 2H), 0.78-0.66 (m, 6H). MS |
| | (ESI, m/e) [M + 1]⁺ 594.5. |
| 73 | ¹H NMR (400 MHz, DMSO-d₆) δ ppm: 10.11 |
| | (s, 1H), 8.48 (d, J = 8.0 Hz, 1H), 7.64-7.30 (m, |
| | 4H), 7.12 (d, J = 7.6 Hz, 1H), 7.06 (s, 1H), |
| | 6.78 (s, 1H), 4.42-4.35 (m, 1H), 4.02 (s, 3H), |
| | 3.83-3.74 (m, 1H), 3.53-3.38 (m, 3H), 3.22- |
| | 3.12 (m, 2H), 3.09-2.99 (m, 1H), 2.59 (d, J = |
| | 4.4 Hz, 3H), 2.56-2.40 (m, 2H), 1.90-1.54 (m, |
| | 6H), 1.21-0.95 (m, 5H). MS (ESI, m/e) [M + 1]⁺ |
| | 604.4. |
| 74 | ¹H NMR (400 MHz, DMSO-d₆) δ ppm: 10.18- |
| | 10.08 (m, 1H), 8.55-8.40 (m, 1H), 7.57-7.48 |
| | (m, 1H), 7.47-7.43 (m, 1H), 7.43-7.32 (m, 1H), |
| | 7.32-7.15 (m, 3H), 7.15-6.95 (m, 4H), 6.72 (s, |
| | 1H), 4.48-4.30 (m, 1H), 4.06-3.90 (m, 3H), |
| | 3.72-3.58 (s, 1H), 3.50-3.38 (m, 1H), 3.16-2.82 |
| | (m, 4H), 2.70-2.60 (m, 1H), 2.59-2.52 (m, 3H), |
| | 2.48-2.38 (m, 1H), 1.95-1.44 (m, 7H), 1.20- |
| | 0.98 (m, 5H). MS (ESI, m/e) [M + 1]⁺ 646.5. |
| 75 | ¹H NMR (400 MHz DMSO-d₆) δ ppm: 10.08 |
| | (s, 1H), 8.45 (d, J = 7.8 Hz, 1H), 7.62-7.25 (m, |
| | 4H), 7.20-6.94 (m, 2H), 6.34 (s, 1H), 4.38 (t, J = |
| | 8.2 Hz, 1H), 4.02 (s, 3H), 3.60-3.32 (m, 3H), |
| | 3.21-3.02 (m, 3H), 2.84-2.68 (m, 2H), 2.54 (d, |
| | J = 3.6 Hz, 3H), 1.83-1.59 (m, 7H), 1.15-0.99 |
| | (m, 7H), 0.71 (d, J = 6.2 Hz, 3H). MS (ESI) |
| | m/e [M + 1]⁺ 564.4. |
| 76 | ¹H NMR (400 MHz, Methanol-d4) δ ppm: |
| | 7.48-7.29 (m, 3H), 7.18-7.11 (m, 1H), 7.02- |
| | 6.84 (m, 5H), 4.44 (d, J = 8.8 Hz, 1H), 4.11- |
| | 4.05 (m, 3H), 3.85-3.75 (m, 1H), 3.67-3.37 (m, |
| | 4H), 3.20-3.07 (m, 3H), 2.85-2.71 (m,3H), |
| | 1.98-1.65 (m, 6H), 1.36-1.07 (m, 6H). MS |
| | (ESI, m/e) [M + 1]⁺ 630.5. |
| 77 | ¹H NMR (400 MHz, DMSO-d₆) δ ppm: 10.15 |
| | (s, 1H), 8.24 (d, J = 8.4 Hz, 1H), 7.53-7.32 (m, |
| | 4H), 7.15-7.05 (m, 3H), 6.75 (s, 1H), 4.30-4.25 |
| | (m, 1H), 3.46-3.41 (m, 2H), 3.22-3.16 (m, 2H), |
| | 2.98-2.96 (m, 1H), 2.64-2.60 ( m, 3H), 1.99- |
| | 1.97 (m, 1H), 1.81-1.67 (m, 7H), 1.53-1.47 (m, |
| | 2H), 1.21-1.02 (m, 5H), 0.65-0.55 (m, 6H). |
| | MS (ESI, m/e) [M + 1]⁺ 573.5. |
| 78 | ¹H NMR (400 MHz, DMSO-d₆) δ ppm: 10.07 |
| | (s, 1H), 8.34 (d, J = 8.2 Hz, 1H), 7.81 (d, J = |
| | 8.0 Hz, 2H), 7.58-7.46 (m, 2H), 7.42-7.29 (m, |
| | 1H), 7.27 (d, J = 8.2 Hz, 2H), 7.10 (d, J = 8.2 |
| | Hz, 1H), 6.75 (s, 1H), 4.44-4.34 (m, 1H), 3.53- |
| | 3.44 (m, 2H), 3.30-3.25 (m 1H), 3.23-3.12 (m, |
| | 2H), 2.95-2.87 (m, 1H), 2.57 (d, J = 4.4 Hz, |
| | 3H), 2.36 (s, 3H), 1.94-1.51 (m, 7H), 1.48-1.41 |
| | (m, 1H), 1.19-1.01 (m, 5H), 0.75-0.68 (m, 6H). |
| | MS (ESI, m/e) [M + 1]⁺ 574.5. |
| 79 | ¹H NMR (400 MHz DMSO-d₆) δ ppm: 10.04 |
| | (s, 1H), 8.83 (d, J = 8.4 Hz, 1H), 8.51 (s, 1H), |
| | 7.92 (s, 2H), 7.67-7.31 (m, 7H), 7.11 (d, J = |
| | 8.0 Hz, 1H), 6.95 (s, 1H), 6.76 (s, 1H), 4.98 (s, |
| | 1H), 3.91 (s, 3H), 3.57-3.16 (m, 6H), 3.11-3.06 |
| | (m, 2H), 2.88 (s, 1H), 2.57 (d, J = 2.8 Hz, 3H), |
| | 1.48 (s, 6H), 1.46-1.39 (m, 1H), 0.77-0.64 (m, |
| | 6H). MS (ESI) m/e [M + 1]⁺ 742.4. |
| 80 | ¹H NMR (400 MHz, DMSO-d₆) δ ppm: 10.13 |
| | (s, 1H), 8.46 (d, J = 7.8 Hz, 1H), 7.59 (d, J = |
| | 7.0 Hz, 2H), 7.53 (d, J = 7.8 Hz, 1H), 7.45 (s, |
| | 1H), 7.38-7.31 (m, 2H), 7.25 (d, J = 7.4 Hz, |
| | 2H), 7.14-7.02 (m, 2H), 6.75 (d, J = 5.2 Hz, |
| | 1H), 4.39 (d, J = 7.6 Hz, 1H), 4.02 (d, J = 3.2 |
| | Hz, 3H), 3.73 (s, 1H), 3.46-3.32 (m, 3H), 3.12- |
| | 3.06 (m, 2H), 2.97 (s, 1H), 2.78 (s, 1H), 2.62 |
| | (s, 1H), 2.55 (d, J = 3.2 Hz, 3H), 1.84 (s, 2H), |
| | 1.69 (s, 2H), 1.60 (s, 2H), 1.15 (d, J = 6.2 Hz, |
| | 3H), 1.00 (d, J = 11.2 Hz, 1H). MS (ESI) m/e |
| | [M + 1]⁺ 680.4 |
| 81 | ¹H NMR (400 MHz, DMSO-d₆) δ ppm: 10.12 |
| | (s, 1H), 8.48 (d, J = 8.0 Hz, 1H), 7.61-7.50 (m, |
| | 2H), 7.46 (d, J = 4.0 Hz, 1H), 7.41-7.31 (m, |
| | 1H), 7.13-7.11 (m, 1H), 7.06 (d, J = 4.0 Hz, |
| | 1H), 6.79 (s, 1H), 4.40-4.35 (m, 1H), 4.02 (s, |
| | 3H), 3.77-3.72 (m, 1H), 3.77-3.72 (m, 1H), |
| | 3.64-3.50 (m, 2H), 3.30-3.24 (m, 2H), 3.19- |
| | 3.12 (m, 1H), 3.05-3.01 (m, 1H), 2.57 (d, J = |
| | 4.0 Hz, 3H), 1.87-1.81 (m, 2H), 1.74-1.70 (m, |
| | 2H), 1.62-1.55 (m, 2H), 1.23-1.15 (m, 6H), |
| | 0.96-0.92 (m, 3H). MS (ESI, m/e) [M + 1]⁺ |
| | 618.5. |
| 82 | ¹H NMR (400 MHz, DMSO-d₆) δ ppm: 10.10 |
| | (s, 1H), 8.48 (d, J = 8.4 Hz, 1H), 7.58-7.30 (m, |
| | 4H), 7.12 (d, J = 8.4 Hz, 1H), 7.06 (s, 1H), |
| | 6.75 (s, 1H), 4.48-4.43 (m, 1H), 4.01 (s, 3H), |
| | 3.57-3.45 (m, 2H), 3.25-3.12 (m, 4H), 2.95- |
| | 2.89 (m, 1H), 2.13-2.06 (m, 1H), 1.77-1.28 (m, |
| | 14H), 0.75 (d, J = 6.4 Hz, 3H), 0.70 (d, J = 6.4 |
| | Hz, 3H). MS (ESI, m/e) [M + 1]⁺ 581.5. |
| 83 | ¹H NMR (400 MHz, DMSO-d₆) δ ppm: 10.10 |
| | (s, 1H), 8.48 (d, J = 8.4 Hz, 1H), 7.57-7.31 (m, |
| | 3H), 7.12 (d, J = 8.4 Hz, 1H), 7.06 (s, 1H), |
| | 7.05-6.93 (m, 2H), 6.77 (s, 1H), 4.48-4.43 (m, |
| | 1H), 4.01 (s, 3H), 3.57-3.35 (m, 4H), 3.25-3.12 |
| | (m, 2H), 2.98-2.89 (m, 1H), 2.13-2.06 (m, 1H), |
| | 1.77-1.28 (m, 14H), 0.76 (d, J = 6.4 Hz, 3H) , |
| | 0.71 (d, J = 6.4 Hz, 3H). MS (ESI, m/e) |
| | [M + 1]⁺ 564.5. |
| 84 | ¹H NMR (400 MHz, DMSO-d₆) δ ppm: 10.08 |
| | (s, 1H), 8.45 (d, J = 7.2 Hz, 1H), 7.57-7.28 (m, |
| | 3H), 7.12 (d, J = 8.4 Hz, 1H), 7.07 (s, 1H), |
| | 6.75 (s, 1H), 4.73-4.65 (m, 1H), 4.43-4.38 (m, |
| | 1H), 3.58-3.44 (m, 2H), 3.32-3.07 (m, 4H), |
| | 2.94-2.87 (m, 1H), 2.58 (d, J = 4.4 Hz, 3H), |
| | 1.95-1.40 (m, 7H), 1.30-1.26 (m, 6H), 1.20- |
| | 1.11 (m, 4H), 1.08-0.95 (m, 1H), 0.74 (d, J = |
| | 6.8 Hz, 3H), 0.70 (d, J = 6.8 Hz, 3H). MS |
| | (ESI, m/e) [M + 1]⁺ 618.5. |
| 85 | ¹H NMR (400 MHz, DMSO-d₆) δ ppm: 10.15 |
| | (s, 1H), 8.54 (d, J = 8.4 Hz, 1H), 7.58-7.43 (m, |
| | 3H), 7.43-7.29 (m, 1H), 7.11 (d, J = 8.2 Hz, |
| | 1H), 7.06 (d, J = 2.0 Hz, 1H), 6.75 (s, 1H), |
| | 4.47-4.35 (m, 1H), 4.03 (s, 3H), 3.93-3.82 (m, |
| | 2H), 3.60-3.42 (m, 2H), 3.26-3.04 (m, 7H), |
| | 2.95-2.85 (m, 1H), 2.57 (d, J = 4.6 Hz, 3H), |
| | 2.15-2.00 (m, 1H), 1.75-1.65 (m, 1H), 1.52- |
| | 1.40 (m, 3H), 0.76-0.66 (m, 6H). MS (ESI, |
| | m/e) [M + 1]⁺ 566.5 |
| 86 | ¹H NMR (400 MHz, DMSO-d₆) δ ppm: 10.09 |
| | (s, 1H), 8.44 (d, J = 8.4 Hz, 1H), 7.45-7.42 (m, |
| | 3H), 7.13-7.10 (m, 4H), 4.39-4.36 (m, 1H), |
| | 4.02 (s, 3H), 3.56-3.51 (s, 4H), 3.26-3.21 (m, |
| | 4H), 2.89-2.85 (m, 1H), 1.91-1.67 (m, 14H), |
| | 1.20-1.04 (m, 4H), 0.84-0.75 (m, 7H). MS |
| | (ESI, m/e) [M + 1]⁺ 648.5. |
| 87 | ¹H NMR (400 MHz, DMSO-d₆) δ ppm: 10.09 |
| | (s, 1H), 8.53 (d, J = 8.4 Hz. 1H), 7.49-7.42 (m, |
| | 4H), 7.08-7.02 (m, 2H), 6.76 (s, 1H), 4.39-4.36 |
| | (m, 1H), 4.02 (s, 3H), 3.46-3.41 (m, 2H), 3.07- |
| | 3.01 (m, 3H), 2.54-2.51 (m, 3H), 1.81-1.67 (m, |
| | 2H), 1.51-1.17 (m, 7H), 1.05-1.02 (m, 2H), |
| | 0.79-0.72 (m, 6H), 0.63-0.55 (m, 6H). MS |
| | (ESI, m/e) [M + 1]⁺ 592.5. |
| 88 | ¹H NMR (400 MHz, DMSO-d₆) δ ppm: 10.10 |
| | (s, 1H), 8.43 (d, J = 8.4 Hz. 1H), 7.49-7.42 (m, |
| | 4H), 7.08-7.02 (m, 2H), 6.65 (s, 1H), 4.40-4.36 |
| | (m, 1H), 4.02 (s, 3H), 3.46-3.41 (m, 4H), 3.07- |
| | 3.01 (m, 2H), 2.78-2.76 (m, 1H), 2.54-2.51 (m, |
| | 3H).1.81-1.67 (m, 11H), 1.05-1.02 (m, 8H), |
| | 0.79-0.72 (m, 2H). MS (ESI, m/e) [M + 1]⁺ |
| | 618.6. |
| 89 | ¹H NMR (400 MHz, DMSO-d₆) δ ppm: 10.10 |
| | (s, 1H), 8.49 (d, J = 8.0 Hz, 1H), 7.58-7.30 (m, |
| | 4H), 7.12 (d, J = 8.0 Hz, 1H), 7.07 (s, 1H), |
| | 6.75 (s, 1H), 4.40-4.34 (m, 1H), 4.02 (s, 3H), |
| | 3.59-3.46 (m, 2H), 3.27-3.12 (m, 4H), 2.95- |
| | 2.87 (m, 1H), 2.58 (d, J = 4.4 Hz, 3H), 1.90- |
| | 1.54 (m, 5H), 1.50-1.25 (m, 3H), 1.21-0.85 (m, |
| | 6H), 0.75 (d, J = 6.8 Hz, 3H), 0.70 (d, J = 6.4 |
| | Hz, 3H). MS (ESI, m/e) [M + 1]⁺ 578.5. |
| 90 | ¹H NMR (400 MHz, DMSO-d₆) δ ppm: 10.07 |
| | (s, 1H), 8.42 (d, J = 8.8 Hz, 1H), 7.52-7.46 (m, |
| | 4H), 7.17 (d, J = 8.4 Hz, 1H), 7.05 (s, 1H), |
| | 6.74 (s, 1H), 4.82-4.78 (m, 1H), 4.05 (s, 3H), |
| | 3.50-3.47 (m, 2H), 3.28-3.21 (m, 3H), 3.03- |
| | 2.98 (m, 1H), 2.58-2.52 (m, 3H), 1.49-1.34 (m, |
| | 1H), 0.93-0.68 (m, 9H), 0.45-0.07 (m, 8H). |
| | MS (ESI, m/e) [M + 1]⁺ 576.5. |
| 91 | ¹H NMR (400 MHz, DMSO-d₆) δ ppm: 10.05 |
| | (s, 1H), 8.42 (d, J = 8.8 Hz, 1H), 7.52-7.46 (m, |
| | 4H), 7.17 (d, J = 8.4 Hz, 1H), 6.93 (d, J = 1.8 |
| | Hz, 1H), 6.74 (s, 1H), 5.48-5.34 (m, 1H), 4.82- |
| | 4.78 (m, 1H), 3.50-3.47 (m, 2H), 3.28-3.21 (m, |
| | 3H), 3.17-3.15 (m, 1H), 3.03-2.98 (m, 1H), |
| | 2.58-2.52 (m, 3H), 1.49-1.34 (m, 7H), 0.93- |
| | 0.68 (m, 10H), 0.45-0.07 (m, 8H). MS (ESI, |
| | m/e) [M + 1]⁺ 604.5. |
| 92 | ¹H NMR (400 MHz, DMSO-d₆) δ ppm: 10.10 |
| | (d, J = 8.8 Hz, 1H), 8.47 (d, J = 8.0 Hz, 1H), |
| | 7.59-7.30 (m, 4H), 7.18-7.00 (m, 6H), 6.82 (d, |
| | J = 4.4 Hz, 1H), 4.44-4.37 (m, 1H), 4.02 (s, |
| | 3H), 3.74-3.46 (m, 3H), 3.27-3.00 (m, 5H), |
| | 2.86-2.65 (m, 2H), 2.58-2.54 (m, 3H), 2.04- |
| | 1.54 (m, 6H), 1.20-0.95 (m, 5H). MS (ESI, |
| | m/e) [M + 1]⁺ 638.5. |
| 93 | ¹H NMR (400 MHz, DMSO-d₆) δ ppm: 10.07 |
| | (s, 1H), 8.40 (d, J = 8.8 Hz, 1H), 7.57-7.50 (m, |
| | 1H), 7.50-7.43 (m, 2H), 7.40-7.32 (m, 1H), |
| | 7.11 (d, J = 7.8 Hz, 1H), 7.08-7.02 (m, 1H), |
| | 6.74 (s, 1H), 4.85-4.74 (m, 1H), 4.04 (s, 3H), |
| | 3.61-3.45 (m, 2H), 3.29-3.27 (m, 1H), 3.25- |
| | 3.12 (m, 2H), 2.92-2.82 (m, 1H), 2.60-2.55 (m, |
| | 3H), 2.04-1.91 (m, 1H), 1.53-1.35 (m, 1H), |
| | 0.85-0.65 (m, 9H), 0.48-0.05 (m, 8H). MS |
| | (ESI, m/e) [M + 1]⁺ 576.5. |
| 94 | ¹H NMR (400 MHz, DMSO-d₆) δ ppm: 10.07 |
| | (s, 1H), 8.44 (d, J = 8.4 Hz, 1H), 7.53 (d, J = |
| | 19.0 Hz, 1H), 7.45 (d, J = 2.0 Hz, 1H), 7.39- |
| | 7.28 (m, 2H), 7.10 (d, J = 8.2 Hz, 1H), 7.08- |
| | 7.03 (m, 1H), 6.34 (s, 1H), 4.44-4.30 (m, 1H), |
| | 4.02 (s, 3H), 3.66-3.52 (m, 1H), 3.47-3.38 (m, |
| | 1H), 3.21-2.81 (m, 6H), 2.54 (d, J = 4.6 Hz, |
| | 3H), 1.93-1.52 (m, 6H), 1.21-0.94 (m, 6H), |
| | 0.54-0.41 (m, 1H), 0.39-0.29 (m, 1H), 0.29- |
| | 0.18 (m, 1H), 0.11-0.04 (m, 1H), 0.00--0.1 (m, |
| | 1H). MS (ESI, m/e) [M + 1]⁺ 576.5. |
| 95 | ¹H NMR (400 MHz, DMSO-d₆) δ ppm: 10.06 |
| | (s, 1H), 8.43 (d, J = 8.4 Hz, 1H), 7.48-7.43 (m, |
| | 2H), 7.40-7.34 (m, 1H), 7.34-7.28 (m, 1H), |
| | 7.09 (d, J = 8.4 Hz, 1H), 7.05 (d, J = 2.0 Hz, |
| | 1H), 6.32 (s, 1H), 4.42-4.34 (m, 1H), 4.02 (s, |
| | 3H), 3.58-3.40 (m, 2H), 3.22-3.02 (m, 4H), |
| | 2.54 (d, J = 4.6 Hz, 3H), 2.02-1.50 (m, 8H), |
| | 1.43-1.32 (m, 1H), 1.23-1.07 (m, 4H), 1.07- |
| | 0.95 (m, 4H). MS (ESI, m/e) [M + 1]⁺ 550.5. |
| 96 | ¹H NMR (400 MHz, DMSO-d₆) δ ppm: 10.13- |
| | 10.10 (m, 1H), 8.47 (d, J = 8.0 Hz, 1H), 7.56- |
| | 7.49 (m, 2H), 7.46-7.45 (m, 1H), 7.43-7.33 (m, |
| | 1H), 7.26-7.21 (m, 1H), 7.16-7.10 (m, 4H), |
| | 7.07-7.06 (m, 1H), 6.79 (s, 1H), 4.41-4.36 (m, |
| | 1H), 4.02 (d, J = 4.0 Hz, 3H), 3.54-3.40 (m, |
| | 4H), 3.26-3.13 (m, 2H), 2.93-2.88 (m, 1H), |
| | 2.58 (d, J = 4.0 Hz, 3H), 2.47-2.45 (m, 1H), |
| | 1.87-1.81 (m, 2H), 1.73-1.69 (m, 2H), 1.63- |
| | 1.56 (m, 2H), 1.23-1.15 (m, 5H). MS (ESI, |
| | m/e) [M + 1]⁺ 626.5. |
| 97 | ¹H NMR (400 MHz, DMSO-d₆) δ ppm: 10.28 |
| | (s, 0.19H), 10.02 (s, 0.78H), 9.03 (d, J = 8.0 Hz, |
| | 0.2H), 8.65 (d, J = 8.0 Hz, 0.8H), 7.63-7.28 (m, |
| | 5H), 7.22-7.05 (m, 4H), 7.01-6.99 (m, 0.8H), |
| | 6.81 (s, 1H), 4.96-4.92 (m, 0.8H), 4.66-4.61 (m, |
| | 0.2H), 4.05-4.02 (m, 3H), 3.85-3.80 (m, 1H), |
| | 3.65-3.52 (m, 2H), 3.31-3.19 (m, 2H), 3.08-2.82 |
| | (m, 3H), 2.66-2.62 (m, 3H), 2.20-2.03 (m, 2H), |
| | 1.53-1.48 (m, 2H), 0.81-0.75 (m, 6H). MS (ESI, |
| | m/e) [M + 1]⁺ 598.5. |
| 98 | ¹H NMR (400 MHz, DMSO-d₆) δ ppm: 10.03 |
| | (s, 1H), 8.43 (d, J = 8.4 Hz. 1H), 7.49-7.42 (m, |
| | 4H), 7.08-7.02 (m, 2H), 6.36 (s, 1H), 4.39-4.36 |
| | (m, 1H), 4.02 (s, 3H), 3.46-3.41 (m, 2H), 3.07- |
| | 3.01 (m, 4H), 2.98-2.96 (m, 2H), 2.54-2.51 (m, |
| | 3H).1.81-1.67 (m, 7H), 1.05-1.02 (m, 6H), |
| | 0.79-0.72 (m, 3H). MS (ESI, m/e) [M + 1]⁺ |
| | 550.5. |
| 99 | ¹H NMR (400 MHz, DMSO-d₆) δ ppm: 10.10 |
| | (d, J = 4.0 Hz, 1H), 8.47 (d, J = 7.4 Hz, 1H), |
| | 7.51 (s, 1H), 7.46 (s, 1H), 7.38-7.33 (m, 2H), |
| | 7.22-7.18 (m, 2H), 7.15-7.03 (m, 3H), 7.00- |
| | 6.95 (m, 2H), 6.35 (s, 1H), 4.40 (t, J = 8.4 Hz, |
| | 1H), 4.03 (s, 3H), 3.62 (t, J = 15.6 Hz, 1H), |
| | 3.37-3.32 (m, 1H), 3.17-3.01 (m, 5H), 2.97 (s, |
| | 1H), 2.83-2.75 (m, 2H), 2.54 (d, J = 4.0 Hz, |
| | 3H), 2.43-2.41 (m, 2H), 2.05 (s, 1H), 1.85 (s, |
| | 2H), 1.70 (s, 2H), 1.61 (s, 2H), 1.23-1.16 (m, |
| | 3H), 1.05-0.99 (m, 1H). MS (ESI) m/e [M + 1]⁺ |
| | 626.4 |
| 100 | ¹H NMR (400 MHz, DMSO-d₆) δ ppm: 10.09 |
| | (s, 1H), 8.45-8.43(m, 1H), 7.56-7.27 (m, 4H), |
| | 7.11-7.06 (m, 2H), 6.89-6.66 (m, 4H), 6.41 (d, |
| | J = 7.4 Hz, 1H), 4.39 (t, J = 74 Hz, 1H), 4.02 |
| | (d, J = 5.8 Hz, 3H), 3.75-3.62 (m, 4H), 3.50- |
| | 3.38 (m, 2H), 3.20-2.91 (m, 5H), 2.80 (d, J = |
| | 11.2 Hz, 1H), 2.68 (d, J = 9.8 Hz, 1H), 2.55 (d, |
| | J = 4.4 Hz, 3H), 2.40 (d, J = 13.4 Hz, 1H), |
| | 2.24 (d, J = 13.4 Hz, 1H), 1.84 (s, 2H), 1.70 (s, |
| | 2H), 1.59 (s, 2H), 1.16 (s, 2H), 1.02 (s, 1H), |
| | 0.64-0.55 (m, 3H). MS (ESI) m/e [M + 1]⁺ |
| | 670.4. |
| 101 | ¹H NMR (400 MHz, DMSO-d₆) δ ppm: 10.06 |
| | (s, 1H), 8.44 (d, J = 8.0 Hz, 1H), 7.55-7.47 (m, |
| | 2H), 7.45 (d, J = 4.0 Hz, 1H), 7.40-7.29 (m, |
| | 1H), 7.11 (d, J = 8.0 Hz, 1H), 7.05 (d, J = 4.0 |
| | Hz, 1H), 6.74 (s, 1H), 4.38-4.34 (m, 1H), 4.03- |
| | 4.02 (m, 3H), 3.57-3.45 (m, 2H), 3.23-3.12 (m, |
| | 3H), 2.92-2.89 (m, 1H), 2.58 (d, J = 4.0 Hz, |
| | 3H), 1.88-1.75 (m, 4H), 1.61-1.58 (m, 1H), |
| | 1.47-1.42 (m, 2H), 1.24-1.15 (m, 5H), 1.07-1.00 |
| | (m, 2H), 0.85-0.82 (m, 3H), 0.85-0.82 (m, 3H), |
| | 0.75-0.68 (m, 6H). MS (ESI, m/e) [M + 1]⁺ |
| | 592.6. |
| 102 | ¹H NMR (400 MHz, DMSO-d₆) δ ppm: 10.06 (s, |
| | 1H), 8.44 (d, J = 8.2 Hz, 1H), 7.53 (s, 1H), 7.45 |
| | (d, J = 2.0 Hz, 1H), 7.33-7.29 (m, 2H), 7.11- |
| | 7.02 (m, 2H), 6.42 (s, 1H), 4.38 (t, J = 8.4 Hz, |
| | 1H), 4.03 (s, 3H), 3.55-3.45 (m, 2H), 3.21-2.98 |
| | (m, 5H), 2.85 (s, 2H), 2.56 (d, J = 4.4 Hz, 3H), |
| | 1.88-1.52 (m, 6H), 1.35-1.01 (m, 5H), 0.39 (s, |
| | 2H), 0.24-0.11 (m, 2H). MS (ESI, m/e) [M + 1]⁺ |
| | 562.5 |
| 103 | ¹H NMR (400 MHz, DMSO-d₆) δ ppm: 10.14 (s, |
| | 1H), 8.39-8.31 (m, 1H), 7.80 (d, J = 2.2 Hz, |
| | 1H), 7.64 (d, J = 8.8 Hz, 1H), 7.58-7.51 (m, |
| | 1H), 7.39-7.31 (m, 1H), 7.11 (d, J = 8.6 Hz, 1H), |
| | 6.74 (s, 1H), 6.65 (d, J = 2.2 Hz, 1H), 4.7- |
| | 4.92(m, 1H), 4.56-4.52 (m, 1H), 4.25-4.21 (m, |
| | 2H), 3.85-3.75 (m, 2H), 3.71-3.51 (m, 4H), 3.9- |
| | 3.05 (m, 4H), 2.65-2.55 (m, 3H), 1.81-1.61 (m, |
| | 6H), 1.35-1.1 (s, 5H), 0.85-0.69 (m, 6H). MS |
| | (ESI, m/e) [M + 1]⁺ 594.8. |
| 104 | ¹H NMR (400 MHz, DMSO-d₆) δ ppm: 10.07 |
| | (s, 1H), 8.44 (d, J = 8.0 Hz, 1H), 7.56-7.49 (m, |
| | 2H), 7.46-7.44 (m, 1H), 7.40-7.30 (m, 1H), 7.11 |
| | (d, J = 8.0 Hz, 1H), 7.07-7.05 (m, 1H), 6.75 (s, |
| | 1H), 4.39-4.34 (m, 1H), 4.04-4.01 (m, 3H), |
| | 3.57-3.45 (m, 2H), 3.23-3.12 (m, 2H), 2.94-2.88 |
| | (m, 1H), 2.58 (d, J = 4.0 Hz, 3H), 1.90-1.78 (m, |
| | 2H), 1.74-1.69 (m, 1H), 1.65-1.54 (m, 2H), |
| | 1.49-1.40 (m, 1H), 1.35-1.20 (m, 4H), 1.10-0.99 |
| | (m, 1H), 0.87-0.82 (m, 5H), 0.74-0.68 (m, 6H). |
| | MS (ESI, m/e) [M + 1]⁺ 578.5. |
| 105 | ¹H NMR (400 MHz, DMSO-d₆) δ ppm: 10.08 |
| | (s, 1H), 8.46 (d, J = 8.0 Hz, 1H), 7.57-7.28 (m, |
| | 4H), 7.12 (d, J = 8.0 Hz, 1H), 7.06 (s, 1H), |
| | 6.33 (s, 1H), 4.44-4.37 (m, 1H), 4.03 (s, 3H), |
| | 3.59-3.46 (m, 2H), 3.17-3.06 (m, 2H), 2.98- |
| | 2.92 (m, 2H), 2.78-2.70 (m, 2H), 2.58 (d, J = |
| | 4.4 Hz, 3H), 1.90-1.54 (m, 6H), 1.20-0.95 (m, |
| | 7H), 0.65-0.55 (m, 6H). MS (ESI, m/e) [M + 1]⁺ |
| | 578.5. |
| 106 | ¹H NMR (400 MHz, DMSO-d₆) δ ppm: 10.13 |
| | (s, 1H), 8.43 (d, J = 8.4 Hz, 1H), 7.49-7.42 (m, |
| | 3H), 7.42-7.35 (m, 2H), 7.06 (s, 1H), 6.36 (s, |
| | 1H), 4.39-4.36 (m, 1H), 4.02 (s, 3H), 3.46-3.41 |
| | (m, 4H), 3.22-3.16 (m, 2H), 2.98-2.96 (m, 1H), |
| | 2.54-2.52(m, 3H), 2.24-2.22 (m, 2H), 1.81- |
| | 1.67 (m, 2H), 1.21-1.02 (m, 5H), 1.16-1.14 (m, |
| | 5H). MS (ESI, m/e) [M + 1]⁺ 574.5. |
| 107 | ¹H NMR (400 MHz, DMSO-d₆) δ ppm: 10.09 |
| | (s, 1H), 8.44 (d, J = 8.4 Hz, 1H), 7.45-7.42 (m, |
| | 3H), 7.13-7.10 (m, 4H), 4.39-4.36 (m, 1H), |
| | 4.02 (s, 3H), 3.56-3.51 (s, 3H), 3.46-3.41 (m, |
| | 3H), 1.91-1.67 (m, 17H), 1.20-1.04 (m, 6H), |
| | 0.84-0.75 (m, 7H). MS (ESI, m/e) [M + 1]⁺ |
| | 662.6 |
| 108 | ¹H NMR (400 MHz, DMSO-d₆) δ ppm: 10.09 |
| | (s, 1H), 8.45 (d, J = 7.6 Hz, 1H), 7.55 (s, 1H), |
| | 7.45 (s, 1H), 7.38 (d, J = 3.8 Hz, 1H), 7.33 (d, |
| | J = 8.0 Hz, 1H), 7.12 (d, J = 8.0 Hz, 1H), 7.06 |
| | (s, 1H), 6.31 (s, 1H), 4.39 (t, J = 8.4 Hz, 1H), |
| | 4.02 (s, 3H), 3.57-3.47 (m, 2H), 3.20-2.91 (m, |
| | 6H), 2.55 (d, J = 4.2 Hz, 3H), 1.82 (d, J = 10.8 |
| | Hz, 2H), 1.77-1.52 (m, 8H), 1.45-1.39 (m, |
| | 2H), 1.19-1.16 (m, 5H). MS (ESI) m/e [M + 1]⁺ |
| | 576.4. |
| 109 | ¹H NMR (400 MHz, DMSO-d₆) δ ppm: 10.17- |
| | 10.02 (m, 1H), 8.46 (d, J = 8.2 Hz, 1H), 7.55- |
| | 7.29 (m, 4H), 7.23-7.05 (m, 5H), 6.92 (t, J = |
| | 5.8 Hz, 2H), 6.42 (s, 1H), 4.40 (t, J = 7.6 Hz, |
| | 1H), 4.03 (d, J = 4.4 Hz, 3H), 3.62-3.56 (m, |
| | 2H), 3.17-2.99 (m, 4H), 2.82 (d, J = 10.0 Hz, |
| | 1H), 2.70 (d, J = 10.0 Hz, 1H), 2.56 (d, J = 4.2 |
| | Hz, 3H), 2.45 (d, J = 12.8 Hz, 1H), 2.29 (d, J = |
| | 12.8 Hz, 1H), 2.01-1.97 (m, 1H), 1.84 (s, 2H), |
| | 1.70 (s, 2H), 1.61 (s, 2H), 1.16 (s, 3H), 1.01 (d, |
| | J = 11.0 Hz, 1H), 0.59 (s, 3H). MS (ESI) m/e |
| | [M + 1]⁺ 640.4. |
| 110 | ¹H NMR (400 MHz, DMSO-d₆) δ ppm: 10.11 |
| | (s, 1H), 9.03 (d, J = 8.2 Hz, 1H), 7.59-6.93 (m, |
| | 4H), 6.31 (s, 1H), 4.47 (t, J = 8.2 Hz, 1H), |
| | 3.56-3.49 (m, 2H), 3.19 (s, 2H), 3.15-3.09 m, |
| | 2H), 2.94 (s, 2H), 2.55 (d, J = 4.4 Hz, 3H), |
| | 2.47 (s, 3H), 1.86-1.50 (m, 10H), 1.43 (s, 2H), |
| | 1.21-1.12 (m, 3H), 1.03 (d, J = 10.4 Hz, 1H), |
| | 0.89-0.80 (m, 1H). MS (ESI) m/e [M + 1]⁺ |
| | 578.4. |
| 111 | [0345] ¹H NMR (400 MHz, DMSO-d₆) δ |
| | ppm: 10.24 (s, 1H), 8.54 (d, J = 8.4 Hz, 1H), |
| | 7.49-7.42 (m, 4H), 7.42-7.35 (m, 1H), 7.13- |
| | 7.10 (m, 2H), 6.76 (m, 1H), 4.92-4.86 (m, 1H), |
| | 4.02 (s, 3H), 3.46-3.41 (m, 2H), 3.22-3.16 (m, |
| | 4H), 2.58-2.56 (m, 3H), 2.40-2.36 (m, 2H), |
| | 1.81-1.67 (m, 11H), 1.55-1.52 (m, 3H), 0.74- |
| | 0.65 (m, 6H). MS (ESI, m/e) [M + 1]⁺ 616.3. |
| 112 | ¹H NMR (400 MHz, DMSO-d₆) δ ppm: 10.03 |
| | (s, 1H), 8.53 (d, J = 8.4 Hz. 1H), 7.49-7.42 (m, |
| | 4H), 7.08-7.02 (m, 2H), 6.36 (s, 1H), 4.39-4.36 |
| | (m, 1H), 4.02 (s, 3H), 3.46-3.41 (m, 2H), 3.07- |
| | 3.01 (m, 3H), 2.98-2.96 (m, 3H), 2.64-2.60(m, |
| | 3H).1.81-1.67 (m, 6H), 1.05-1.02 (m, 3H), |
| | 0.91-0.72 (m, 8H), 0.26-0.14 (m, 3H). MS |
| | (ESI, m/e) [M + 1]⁺ 590.3. |
| 113 | ¹H NMR (400 MHz, DMSO-d₆) δ ppm: 10.03 |
| | (s, 1H), 8.43 (d, J = 8.4 Hz. 1H), 7.49-7.42 (m, |
| | 4H), 7.08-7.02 (m, 2H), 6.36 (s, 1H), 4.39-4.36 |
| | (m, 1H), 4.02 (s, 3H), 3.46-3.41 (m, 2H), 3.07- |
| | 3.01 (m, 3H), 2.98-2.96 (m, 3H), 2.64-2.60 (m, |
| | 3H). 1.81-1.67 (m, 6H), 1.05-1.02 (m, 4H), |
| | 0.51-0.42 (m, 4H), 0.26-0.14 (m, 3H). MS |
| | (ESI, m/e) [M + 1]⁺ 590.4. |
| 114 | ¹H NMR (400 MHz, DMSO-d₆) δ ppm: 10.16 |
| | (d, J = 4.0 Hz, 1H), 8.50 (d, J = 8.0 Hz, 1H), |
| | 7.56-7.49 (m, 2H), 7.44-7.38 (m, 2H), 7.16-7.12 |
| | (m, 2H), 6.48 (s, 1H), 4.88-4.83 (m, 1H), 4.11 |
| | (s, 3H), 3.57 (d, J = 16.0 Hz, 2H), 3.51-3.46 (m, |
| | 1H), 3.16-3.07 (m, 3H), 3.03-2.87 (m, 3H), 2.63 |
| | (d, J = 4.0 Hz, 3H), 0.91-0.77 (m, 3H), 0.51- |
| | 0.31 (m, 6H), 0.25-0.06 (m, 6H). MS (ESI, m/e) |
| | [M + 1]⁺ 574.5 |
| 115 | ¹H NMR (400 MHz, DMSO-d₆) δ ppm: 10.05 |
| | (d, J = 8.0 Hz, 1H), 8.41-8.38 (m, 1H), 7.50 (d, |
| | J = 4.0 Hz, 1.5H), 7.43 (s, 0.5H), 7.38-7.31 (m, |
| | 2H), 7.08 (d, J = 4.0 Hz, 1H), 6.93-6.91(m, 1H), |
| | 6.40 (d, J = 4.0 Hz, 1H), 5.43-5.39 (m, 1H), |
| | 4.80-4.75(m, 1H), 4.54-4.38 (m, 1H), 3.10-3.01 |
| | (m, 4H), 2.57 (d, J = 4.0 Hz, 3H), 2.54 (s, 3H), |
| | 2.03-1.96 (m, 2H), 1.38-1.34 (m, 6H), 0.87-0.84 |
| | (m, 3H), 0.45-0.08 (m, 10H). MS (ESI, m/e) |
| | [M + 1]⁺ 602.4 |
| 116 | ¹H NMR (400 MHz, DMSO-d₆) δ ppm: 10.19 |
| | (s, 1H), 9.78 (d, J = 8.0 Hz, 1H), 7.58-7.45 (m, |
| | 2H), 7.42-7.28 (m, 1H), 7.14 (d, J = 8.4 Hz, |
| | 1H), 6.76 (s, 1H), 4.58-4.53 (m, 1H), 3.60-3.42 |
| | (m, 4H), 3.25-2.82 (m, 2H), 2.94-2.87 (m, 1H), |
| | 2.58 (d, J = 4.8 Hz, 3H), 2.12-1.65 (m, 7H), |
| | 1.52-1.30 (m, 3H), 0.78-0.68 (m, 6H). MS (ESI, |
| | m/e) [M + 1]⁺ 602.4. |
| 117 | ¹H NMR (400 MHz, DMSO-d₆) δ ppm: 10.33 |
| | (s, 1H), 8.53-8.3 (m, 2H), 7.49-7.42 (m, 1H), |
| | 7.42-7.35 (m, 1H), 7.06 (s, 1H), 6.36 (s, 1H), |
| | 4.39-4.36 (m, 1H), 4.02 (s, 3H), 3.46-3.41 (m, |
| | 4H), 3.22-3.16 (m, 2H), 2.98-2.96 (m, 1H), |
| | 2.64-2.60( m, 3H). 1.81-1.67 (m, 8H), 1.21-1.02 |
| | (m, 5H), 1.16-1.14 (m, 6H). MS (ESI, m/e) |
| | [M + 1]⁺ 565.5. |
| 118 | ¹H NMR (400 MHz, DMSO-d₆) δ ppm: 10.06 |
| | (s, 1H), 8.41 (d, J = 8.0 Hz, 1H), 7.55-7.29 (m, |
| | 4H), 7.11 (d, J = 8.1 Hz, 1H), 7.05 (s, 1H), 6.74 |
| | (s, 1H), 4.35 (t, J = 8.4 Hz, 1H), 4.02 (s, 3H), |
| | 3.59-3.43 (m, 2H), 3.22-3.12 (m, 3H), 2.94-2.88 |
| | (m, 2H), 2.57 (d, J = 4.2 Hz, 3H), 1.90 (s, 1H), |
| | 1.76 (s, 3H), 1.65 (d, J = 13.2 Hz, 1H), 1.46- |
| | 1.42 (m, 1H), 1.20-1.10 (m, 2H), 0.94 (s, 3H), |
| | 0.82 (s, 9H), 0.74 (d, J = 6.4 Hz, 3H), 0.69 (d, |
| | J = 6.5 Hz, 3H). MS (ESI) m/e [M + 1]⁺ 620.4. |
| 119 | ¹H NMR (400 MHz, DMSO-d₆) δ ppm: 9.78 (d, |
| | J = 4.8 Hz, 1H), 8.42 (d, J = 8.0 Hz, 1H), 7.60- |
| | 7.45 (m, 3H), 7.10 (d, J = 8.0 Hz, 1H), 7.03 (s, |
| | 1H), 6.77 (s, 1H), 4.56-4.48 (m, 1H), 4.03 (s, |
| | 3H), 3.60-3.42 (m, 3H), 3.25-2.82 (m, 4H), 2.58 |
| | (d, J = 4.4 Hz, 3H), 1.90-1.42 (m, 9H), 1.20- |
| | 1.02 (m, 3H), 0.80-0.68 (m, 6H). MS (ESI, m/e) |
| | [M + 1]⁺ 582.5. |
| 120 | ¹H NMR (400 MHz, DMSO-d₆) δ ppm: 10.11 |
| | (s, 1H), 8.44 (d, J = 8.0 Hz, 1H), 7.55-7.34 (m, |
| | 4H), 7.13 (d, J = 8.0 Hz, 1H), 7.07-7.06 (m, |
| | 1H), 6.29 (s, 1H), 4.82-4.78 (m, 1H), 4.04 (s, |
| | 3H), 3.56-3.45 (m, 2H), 3.19-3.04 (m, 5H), 2.93 |
| | (s, 2H), 2.55 (d, J = 4.0 Hz, 3H), 1.72-1.59 (m, |
| | 3H), 1.53-1.47 (m, 1H), 1.40-1.34 (m, 2H), |
| | 0.87-0.72 (m, 3H), 0.46-0.43 (m, 1H), 0.37-0.34 |
| | (m, 2H), 0.29-0.24 (m, 1H), 0.20-0.16 (m, 2H), |
| | 0.10-0.05 (m, 1H). MS (ESI, m/e) [M + 1]⁺ 588.5 |
| 121 | ¹H NMR (400 MHz, DMSO-d₆) δ ppm: 10.09 |
| | (s, 1H), 8.44 (d, J = 8.4 Hz, 1H), 7.45-7.42 (m, |
| | 3H), 7.13-7.10 (m, 4H), 4.39-4.36 (m, 2H), 4.02 |
| | (s, 3H), 3.56-3.51 (s, 5H), 3.46-3.41 (m, 2H), |
| | 3.27-3.21 (m, 1H), 2.89-2.85 (m, 1H), 1.81-1.67 |
| | (m, 13H), 1.55-1.52 (m, 7H), 1.16-1.14 (m, |
| | 2H), 0.84-0.75 (m, 6H). MS (ESI, m/e) [M + 1]⁺ |
| | 677.2. |
| 122 | ¹H NMR (400 MHz, DMSO-d₆) δ ppm: 10.09 |
| | (s, 1H), 8.44 (d, J = 8.4 Hz, 1H), 7.45-7.42 (m, |
| | 3H), 7.13-7.10 (m, 4H), 4.39-4.36 (m, 2H), 4.02 |
| | (s, 3H), 3.56-3.51 (s, 5H), 3.46-3.41 (m, 2H), |
| | 3.27-3.21 (m, 1H), 2.89-2.85 (m, 1H), 1.81-1.67 |
| | (m, 14H), 1.55-1.52 (m, 7H), 1.16-1.14 (m, |
| | 1H), 0.84-0.75 (m, 6H). MS (ESI, m/e) [M + 1]⁺ |
| | 677.3. |
| 123 | ¹H NMR (400 MHz, DMSO-d₆) δ ppm: 12.11 |
| | (br, 1H), 8.77 (d, J = 8.8 Hz, 1H), 7.60-7.10 (m, |
| | 4H), 7.08-7.05 (m, 1H), 6.52 (s, 1H), 5.06-4.98 |
| | (m, 1H), 4.02 (s, 3H), 3.65-3.56 (m, 1H), 3.25- |
| | 3.10 (m, 3H), 2.85-2.60 (m, 6H), 2.15-2.03 (m, |
| | 2H), 1.90-1.42 (m, 6H), 1.20-0.92 (m, 5H), |
| | 0.84-0.45 (m, 6H). MS (ESI, m/e) [M + 1]⁺ |
| | 575.7. |
| 124 | ¹H NMR (400 MHz, DMSO-d₆) δ ppm: 10.09 |
| | (s, 1H), 8.46 (d, J = 8.4 Hz, 1H), 7.60-7.30 (m, |
| | 4H), 7.10 (d, J = 8.4 Hz, 1H), 7.06 (s, 1H), 6.85 |
| | (s, 1H), 4.45-4.38 (m, 1H), 4.02 (s, 3H), 3.65- |
| | 3.42 (m, 4H), 3.25-2.82 (m, 5H), 1.90-1.42 (m, |
| | 7H), 1.20-0.92 (m, 5H), 0.84-0.70 (m, 12H). |
| | MS (ESI, m/e) [M + 1]⁺ 606.5. |
| 125 | ¹H NMR (400 MHz, DMSO-d₆) δ ppm: 10.08 |
| | (s, 1H), 8.47 (d, J = 8.0 Hz, 1H), 7.58-7.30 (m, |
| | 4H), 7.11 (d, J = 8.0 Hz, 1H), 7.06 (s, 1H), |
| | 6.31(s, 1H), 4.40-4.35 (m, 1H), 4.03 (s, 3H), |
| | 3.58-3.48 (m, 2H), 3.25-3.08 (m, 4H), 2.96-2.92 |
| | (m, 2H), 2.55 (d, J = 4.4 Hz, 3H), 2.10-1.56 (m, |
| | 11H), 1.50-1.38 (m, 3H), 1.22-0.95 (m, 5H), |
| | 0.90-0.78 (m, 3H). MS (ESI, m/e) [M + 1]⁺ |
| | 604.4. |
| 126 | MS (ESI, m/e) [M + 1]⁺ 594.6. |
| 127 | ¹H NMR (400 MHz, DMSO-d₆) δ ppm: 10.10 |
| | (s, 1H), 8.78 (d, J = 8.2 Hz, 1H), 7.46-7.31 (m, |
| | 7H), 7.16-7.12 (m, 1H), 6.95 (s, 2H), 6.75 (s, |
| | 1H), 4.92-4.89 (m, 1H), 3.95 (s, 3H), 3.57-3.38 |
| | (m, 3H), 3.16-3.12 (m, 3H), 2.90-2.87 (m, 1H), |
| | 2.87-2.85 (m, 1H), 1.49-1.47 (m, 1H), 1.16-1.14 |
| | (m, 2H), 0.81-0.69 (m, 6H). MS (ESI, m/e) |
| | [M + 1]⁺ 640.4. |
| 128 | ¹H NMR (400 MHz, DMSO-d₆) δ ppm: 10.23 |
| | (s, 1H), 8.66 (d, J = 8.2 Hz, 1H), 7.51-7.47 (m, |
| | 4H), 7.07-7.00 (m, 6H), 6.76 (s, 1H), 4.52-4.46 |
| | (m, 1H), 4.03 (s, 3H), 3.57-3.38 (m, 2H), 3.16- |
| | 3.12 (m, 3H), 2.90-2.87 (m, 4H), 2.60-2.52 (m, |
| | 4H), 1.84-1.82 (m, 1H), 1.70 (s, 2H), 1.71-1.68 |
| | (m, 1H), 1.49-1.47 (m, 2H), 0.81-0.69 (m, 6H). |
| | MS (ESI, m/e) [M + 1]⁺ 612.8. |
| 129 | ¹H NMR (400 MHz, DMSO-d₆) δ ppm: 10.08 |
| | (s, 1H), 8.45 (d, J = 7.8 Hz, 1H), 7.57-7.46 (m, |
| | 2H), 7.40 (s, 1H), 7.36-7.27 (m, 1H), 7.13-7.08 |
| | (m, 1H), 6.95 (d, J = 1.6 Hz, 1H), 6.75 (s, 1H), |
| | 4.45-4.39 (m, 2H), 3.54-3.44 (m, 2H), 3.22-3.11 |
| | (m, 3H), 2.95-2.85 (m, 2H), 2.57 (d, J = 4.4 Hz, |
| | 3H), 1.87-1.79 (m, 2H), 1.75-1.67 (m, 2H), |
| | 1.67-1.51 (m, 2H), 1.51-1.39 (m, 1H), 1.21-1.11 |
| | (m, 4H), 1.10-0.98 (m, 3H), 0.98-0.87 (m, 2H), |
| | 0.71 (dd, J = 19.4, 6.6 Hz, 6H). MS (ESI, m/e) |
| | [M + 1]⁺ 590.5. |
| 130 | ¹H NMR (400 MHz, DMSO-d₆) δ ppm: 10.01 |
| | (s, 1H), 8.21 (d, J = 8.3 Hz, 1H), 7.76 (d, J = 1.6 |
| | Hz, 1H), 7.51 (s, 1H), 7.44-7.38 (m, 1H), 7.38- |
| | 7.28 (m, 2H), 7.07 (d, J = 8.2 Hz, 1H), 6.42 (s, |
| | 1H), 4.34 (t, J = 8.6 Hz, 1H), 3.85 (s, 3H), 3.57- |
| | 3.45 (m, 2H), 3.12-2.94 (m, 4H), 2.87 (t, J = 7.8 |
| | Hz, 2H), 2.55 (t, J = 4.6 Hz, 3H), 1.87-1.52 (m, |
| | 6H), 1.24-0.92 (m, 5H), 0.39 (s, 2H), 0.19 (t, |
| | J = 9.4 Hz, 2H). MS (ESI, m/e) [M + 1]⁺ 586.4 |
| 131 | ¹H NMR (400 MHz, DMSO-d₆) δ ppm: 10.05 |
| | (s, 1H), 8.43 (d, J = 8.4 Hz, 1H), 7.52 (s, 1H), |
| | 7.39-7.24 (m, 2H), 7.12-7.01 (m, 2H), 6.98 (d, |
| | J = 4.4 Hz, 1H), 6.42 (s, 1H), 4.37 (t, J = 8.6 Hz, |
| | 1H), 3.93 (s, 3H), 3.56-3.38 (m, 2H), 3.15-2.95 |
| | (m, 4H), 2.89-2.79 (m, 2H), 2.56 (d, J = 4.5 Hz, |
| | 3H), 1.91-1.50 (m, 6H), 1.26-0.91 (m, 5H), 0.39 |
| | (s, 2H), 0.18 (q, J = 9.4 Hz, 2H). MS (ESI, m/e) |
| | [M + 1]⁺ 586.5 |
| 132 | ¹H NMR (400 MHz, DMSO-d₆) δ ppm: 10.07 |
| | (s, 1H), 8.46 (d, J = 8.0 Hz, 1H), 7.54-7.52 (m, |
| | 2H), 7.37-7.29 (m, 2H), 7.08 (d, J = 8.0 Hz, |
| | 2H), 7.00 (d, J = 4.0 Hz, 1H), 6.42 (s, 1H), 5.21 |
| | (d, J = 4.0 Hz, 1H), 5.15-5.06 (m, 1H), 4.37- |
| | 4.33 (m, 1H), 3.95-3.86 (m, 1H), 3.54-3.43 (m, |
| | 2H), 3.11-2.95 (m, 4H), 2.88-2.84 (m, 2H), |
| | 2.66-2.60 (m, 2H), 2.56 (d, J = 8.0 Hz, 3H), |
| | 2.43-2.33 (m, 2H), 1.87-1.79 (m, 2H), 1.74-1.69 |
| | (m, 2H), 1.62-1.52 (m, 2H), 1.24-1.14 (m, 4H), |
| | 1.02-0.98 (m, 1H), 0.39 (s, 2H), 0.22-0.15 (m, |
| | 2H). MS (ESI, m/e) [M + 1]⁺ 618.7 |
| 133 | ¹H NMR (400 MHz, DMSO-d₆) δ ppm: 10.15 |
| | (s, 1H), 7.90 (d, J = 4.0 Hz, 1H), 7.64 (d, J = |
| | 12.0 Hz, 1H), 7.52 (s, 1H), 7.35-7.32 (m, 1H), |
| | 7.27 (d, J = 8.0 Hz, 1H), 7.09 (d, J = 12.0 Hz, |
| | 1H), 6.69 (d, J = 4.0 Hz, 1H), 6.41 (s, 1H), 5.35 |
| | (d, J = 4.0 Hz, 1H), 4.55-4.51 (m, 1H), 4.48- |
| | 4.40 (m, 1H), 4.02-3.93 (m, 1H), 3.53-3.44 (m, |
| | 2H), 3.11-3.05 (m, 2H), 3.01-2.96 (m, 2H), 2.85 |
| | (s, 2H), 2.79-2.73 (m, 2H), 2.57 (d, J = 4.0 Hz, |
| | 3H), 2.38-2.30 (m, 2H), 1.77-1.61 (m, 6H), |
| | 1.24-1.08 (m, 4H), 1.00-0.97 (m, 1H), 0.39 (s, |
| | 2H), 0.20-0.17 (m, 2H). MS (ESI, m/e) [M + 1]⁺ |
| | 618.6 |
| 134 | ¹H NMR (400 MHz, DMSO-d₆) δ ppm: 10.10 |
| | (s, 1H), 8.47 (d, J = 8.0 Hz, 1H), 7.55-7.45 (m, |
| | 2H), 7.39-7.29 (m, 2H), 7.10-7.06 (m, 2H), 6.32 |
| | (s, 1H), 4.39-4.35 (m, 1H), 4.02 (s, 3H), 3.53- |
| | 3.44 (m, 2H), 3.14-3.08 (m, 3H), 3.04-2.99 (m, |
| | 2H), 2.55 (d, J = 4.0 Hz, 3H), 2.03-1.97 (m, |
| | 1H), 1.87-1.81 (m, 2H), 1.75-1.67 (m, 4H), |
| | 1.62-1.57 (m, 2H), 1.23-1.15 (m, 5H). MS (ESI, |
| | m/e) [M + 1]⁺ 536.4 |
| 135 | ¹H NMR (400 MHz, DMSO-d₆) δ ppm: 10.08 |
| | (s, 1H), 8.46 (d, J = 8.4 Hz, 1H), 7.56-7.30 (m, |
| | 3H), 7.09 (d, J = 8.4 Hz, 1H), 7.06 (s, 1H), 6.76 |
| | (s, 1H), 4.45-4.38 (m, 1H), 4.02 (s, 3H), 3.68- |
| | 3.42 (m, 3H), 3.25-2.82 (m, 9H), 1.90-1.42 (m, |
| | 7H), 1.20-0.97 (m, 5H), 0.82-0.74 (m, 6H). MS |
| | (ESI, m/e) [M + 1]⁺ 578.6. |
| 136 | ¹H NMR (400 MHz, DMSO-d₆) δ ppm: 10.11 |
| | (s, 1H), 8.49 (d, J = 8.0 Hz, 1H), 7.56-7.28 (m, |
| | 3H), 7.16-7.08 (m, 2H), 6.77-6.72 (m, 2H), |
| | 5.00-4.79 (m, 1H), 4.42-4.19 (m, 2H), 4.02 (s, |
| | 3H), 3.78-3.42 (m, 5H), 3.25-2.86 (m, 4H), |
| | 1.95-1.55 (m, 9H), 1.50-1.36 (m, 1H), 1.20-0.86 |
| | (m, 5H), 0.82-0.70 (m, 6H). MS (ESI, m/e) |
| | [M + 1]⁺ 620.9. |
| 137 | ¹H NMR (400 MHz, DMSO-d₆) δ ppm: 8.78 (d, |
| | J = 8.0 Hz, 1H), 7.46 (d, J = 2.0 Hz, 1H), 7.35- |
| | 7.25 (m, 3H), 7.08 (s, 1H), 6.40 (s, 1H), 5.09- |
| | 5.02 (m, 1H), 4.05 (s, 3H), 3.68-3.55 (m, 2H), |
| | 3.25-2.82 (m, 6H), 2.56 (d, J = 4.8 Hz, 3H), |
| | 2.15-1.62 (m, 8H), 1.40-1.32 (m, 1H), 0.45-0.14 |
| | (m, 4H). MS (ESI, m/e) [M + 1]⁺ 559.6. |
| 138 | ¹H NMR (400 MHz, DMSO-d₆) δ ppm: 10.18 |
| | (s, 1H), 9.32 (s, 1H), 7.52-7.25 (m, 3H), 7.12- |
| | 7.06 (m, 1H), 6.65 (s, 1H), 4.57-4.53 (m, 2H), |
| | 3.65-3.30 (m, 4H), 3.28-3.10 (m, 7H), 2.49-2.45 |
| | (m, 3H), 2.01-1.63 (m, 9H), 1.55-1.43 (m, 2H), |
| | 0.78-0.70 (m, 2H), 0.58-0.41 (m, 2H). MS (ESI, |
| | m/e) [M + 1]⁺ 600.4 |
| 139 | ¹H NMR (400 MHz, DMSO-d₆) δ ppm: 8.75 (d, |
| | J = 8.8 Hz, 1H), 7.46 (d, J = 6.0 Hz, 1H), 7.40- |
| | 7.30 (m, 3H), 7.07 (s, 1H), 6.29 (s, 1H), 5.07- |
| | 5.02 (m, 1H), 4.01 (s, 3H), 3.68-3.58 (m, 2H), |
| | 3.25-3.06 (m, 6H), 2.95-2.92 (m, 2H), 2.55 (d, |
| | J = 4.8 Hz, 3H), 2.10-1.56 (m, 8H), 1.40-1.32 |
| | (m, 3H), 1.22-0.85 (m, 7H). MS (ESI, m/e) |
| | [M + 1]⁺ 590.6. |
| 140 | ¹H NMR (400 MHz, DMSO-d₆) δ ppm: 10.19- |
| | 10.04 (m, 1H), 8.46 (t, J = 7.2 Hz, 1H), 7.71- |
| | 7.67 (m, 2H), 7.55-7.34 (m, 3H), 7.32-7.18 (m, |
| | 3H), 7.12-7.01 (m, 2H), 6.74 (d, J = 3.0 Hz, |
| | 1H), 4.39 (d, J = 3.6 Hz, 1H), 4.07-4.02 (m, |
| | 3H), 3.73 (d, J = 5.2 Hz, 1H), 3.54-3.45 (m, |
| | 1H), 3.10-3.06 (m, 4H), 2.95 (s, 1H), 2.79-2.73 |
| | (m, 1H), 2.68-2.60 (m, 1H), 2.55 (d, J = 2.6 Hz, |
| | 3H), 1.84 (s, 2H), 1.70 (s, 2H), 1.60 (s, 2H), |
| | 1.19-1.16 (m, 4H), 1.06-0.99 (m, 1H). MS (ESI) |
| | m/e [M + 1]⁺ 637.5. |
| 141 | ¹H NMR (400 MHz, DMSO-d₆) δ ppm: 10.07 |
| | (s, 1H), 8.45 (d, J = 8.4 Hz, 1H), 7.53 (s, 1H), |
| | 7.41 (d, J = 1.8 Hz, 1H), 7.31 (dd, J = 14.2, 6.6 |
| | Hz, 2H), 7.08 (d, J = 8.2 Hz, 1H), 6.96 (d, J = |
| | 1.8 Hz, 1H), 6.42 (s, 1H), 4.45-4.38 (m, 2H), |
| | 3.54-3.43 (m, 2H), 3.11-2.95 (m, 4H), 2.86 (s, |
| | 2H), 2.56 (d, J = 4.6 Hz, 3H), 1.85-1.59 (m, |
| | 6H),1.27-1.11 (m, 4H), 1.06-0.90 (m, 5H), 0.39 |
| | (s, 2H), 0.20-0.16 (m, 2H). MS (ESI, m/e) |
| | [M + 1]⁺ 588.5 |
| 142 | ¹H NMR (400 MHz, DMSO-d₆) δ ppm: 10.15 |
| | (s, 1H), 8.61 (d, J = 8.8 Hz, 1H), 7.53-7.30 (m, |
| | 4H), 7.09 (d, J = 8.4 Hz, 1H), 7.06 (s, 1H), 6.42 |
| | (s, 1H), 4.53-4.43 (m, 1H), 4.03 (s, 3H), 3.58- |
| | 3.42 (m, 2H), 3.25-2.82 (m, 6H), 2.56 (d, J = |
| | 4.8 Hz, 3H), 2.10-1.62 (m, 8H), 1.50-1.32 (m, |
| | 2H), 0.45-0.14 (m, 4H). MS (ESI, m/e) [M + 1]⁺ |
| | 598.5. |
| 143 | ¹H NMR (400 MHz, DMSO-d₆) δ ppm: 9.93 (s, |
| | 1H), 8.31 (d, J = 8.4 Hz, 1H), 7.38-7.29 (m, |
| | 2H), 7.26-7.10 (m, 2H), 6.93 (d, J = 8.2 Hz, |
| | 1H), 6.91-6.89 (m, 1H), 6.27 (s, 1H), 4.24 (t, J = |
| | 8.4 Hz, 1H), 3.88 (s, 3H), 3.39-3.28 (m, 2H), |
| | 2.98-2.78 (m, 4H), 2.75-2.67 (m, 2H), 2.41 (d, |
| | J = 4.6 Hz, 3H), 1.83-1.47 (m, 6H), 1.38 (t, J = |
| | 19.4 Hz, 3H), 1.05-0.83 (m, 4H), 0.28-0.21 (m, |
| | 2H), 0.08-0.01 (m, 2H). MS (ESI, m/e) [M + 1]⁺ |
| | 626.4. |
| 144 | ¹H NMR (400 MHz, DMSO-d₆) δ ppm: 10.07 |
| | (d, J = 4.4 Hz, 1H), 8.43 (d, J = 8.4 Hz, 1H), |
| | 7.63-7.25 (m, 3H), 7.25-6.91 (m, 3H), 6.57 (s, |
| | 1H), 4.38 (t, J = 8.6 Hz, 1H), 4.03 (s, 3H), 3.82 |
| | (s, 1H), 3.70 (t, J = 14.8 Hz, 1H), 3.44 (s, 1H), |
| | 3.41-3.36 (m, 1H), 3.19-3.12(m, 1H), 3.06-2.95 |
| | (m, 1H), 2.56-2.53 (m, 3H), 1.84-1.70 (m, 6H), |
| | 1.64-1.44 (m, 6H), 1.21-1.10 (m, 4H), 1.05-0.96 |
| | (t, J = 11.8 Hz, 1H). MS (ESI, m/e) [M + 1]⁺ |
| | 562.5 |
| 146 | ¹H NMR (400 MHz, DMSO-d₆) δ ppm: 10.09 |
| | (s, 1H), 8.46 (d, J = 8.0 Hz, 1H), 7.61 (s, 1H), |
| | 7.43 (s, 2H), 7.18-7.02 (m, 2H), 7.05 (s, 1H), |
| | 4.57 (s, 1H), 4.39-4.37 (m, 2H), 4.03 (s, 3H), |
| | 3.23 (s, 1H), 2.98-2.90 (m, 5H), 1.84-1.82 (m, |
| | 2H), 1.70-1.59 (s, 4H), 1.14-1.02 (m, 3H). MS |
| | (ESI, m/e) [M + 1]⁺ 533.4. |
| 147 | ¹H NMR (400 MHz, DMSO-d₆) δ ppm: 10.06 |
| | (s, 1H), 8.44 (d, J = 8.4 Hz, 1H), 7.49-7.42 (m, |
| | 4H), 7.42-7.35 (m, 2H), 6.36 (s, 1H), 4.39-4.36 |
| | (m, 1H), 4.02 (s, 3H), 3.46-3.41 (m, 3H), 3.22- |
| | 3.16 (m, 3H), 2.98-2.96 (m, 3H), 2.64-2.60( m, |
| | 3H).1.81-1.67 (m, 8H), 1.55-1.52 (m, 3H), |
| | 1.16-1.14 (m, 4H). MS (ESI, m/e) [M + 1]⁺ |
| | 590.5. |
| 148 | ¹H NMR (400 MHz, DMSO-d₆) δ ppm: 10.13 |
| | (s, 1H), 8.48 (d, J = 8.0 Hz, 1H), 7.55-7.51 (m, |
| | 1H), 7.47-7.44 (m, 1H), 7.41-7.31 (m, 1H), |
| | 7.11-7.07 (m, 2H), 6.47 (s, 1H), 4.38-4.33 (m, |
| | 1H), 4.04-4.02 (m, 3H), 3.54 (s, 3H), 3.47-3.37 |
| | (m, 2H), 3.19-3.12 (m, 2H), 3.00-2.98 (m, 2H), |
| | 2.86 (s, 2H), 2.01-1.61 (m, 6H), 1.40-1.36 (m, |
| | 2H), 1.02-0.91 (m, 3H), 0.85-0.82 (m, 6H), |
| | 0.46-0.44 (m, 2H), 0.32-0.26 (m, 2H). MS (ESI, |
| | m/e) [M + 1]⁺ 605.5 |
| 149 | ¹H NMR (400 MHz, DMSO-d₆) δ ppm: 12.23 (s, |
| | 1H), 10.03 (s, 1H), 8.44 (d, J = 8.2 Hz, 1H), |
| | 7.60-7.45 (m, 3H), 7.41-7.35 (m, 2H), 7.10 (d, |
| | J = 8.6 Hz, 1H), 6.98 (d, J = 8.6 Hz, 1H), 6.74 |
| | (s, 1H), 4.38 (t, J = 8.8 Hz, 1H), 3.59 -- 3.49 (m, |
| | 2H), 3.24-3.16 (m, 2H), 2.90 (t, J = 7.8 Hz, 2H), |
| | 2.57 (d, J = 4.6 Hz, 3H), 2.12-1.82 (m, |
| | 9H),1.78-1.68(m,2H) 1.68-1.61 (m, 5H), 1.52- |
| | 1.42 (m, 2H), 1.19-1.11 (m, 5H), 0.75-0.68 (m, |
| | 6H). MS (ESI, m/e) [M + 1]⁺ 670.7 |
| 150 | ¹H NMR (400 MHz, DMSO-d₆) δ ppm: 10.09 |
| | (s, 1H), 8.45 (d, J = 8.4 Hz, 1H), 8.04-8.02 (m, |
| | 1H), 7.57 (s, 0.5H), 7.48 (s, 0.5H), 7.45 (d, J = |
| | 2.0 Hz, 1H), 7.41 (d, J = 8.2 Hz, 0.5H), 7.32 (d, |
| | J = 7.8 Hz, 0.5H), 7.15-7.12 (m, 3H), 7.06 (d, J = |
| | 2.0 Hz, 1H), 6.85-6.78 (m, 3H), 4.39-4.35 (m, |
| | 1H), 4.23-4.19 (m, 2H), 4.02 (s, 3H), 3.64-3.34 |
| | (m, 4H), 3.21-3.18 (m, 2H), 2.96-2.85 (m, 1H), |
| | 1.85-1.82 (m, 2H), 1.72-1.70 (m, 2H), 1.61-1.58 |
| | (m, 2H), 1.47-1.35 (m, 1H), 1.22-1.18 (m, 5H), |
| | 1.02-0.98 (m, 1H), 0.75-0.68 (m, 6H). MS (ESI, |
| | m/e) [M + 1]⁺ 670.6. |
| 151 | ¹H NMR (400 MHz, DMSO-d₆) δ ppm: 10.10 |
| | (s, 1H), 9.45 (d, J = 3.4 Hz, 1H), 8.44 (d, J = 8.2 |
| | Hz, 1H), 7.62-7.55 (m, 3H), 7.51 (s, 0.5H), |
| | 7.45-7.42 (m, 1H), 7.35-7.24 (m, 3H), 7.16-7.12 |
| | (m, 1H), 7.07-7.00 (m, 2H), 6.92 (s, 1H), 4.42- |
| | 4.36 (m, 1H), 4.03 (s, 3H), 3.77-3.64 (m, 1H), |
| | 3.57-3.38 (m, 3H), 3.16-3.12 (m, 1H), 2.90-2.87 |
| | (m, 1H), 1.84-1.82 (m, 2H), 1.70 (s, 2H), 1.61- |
| | 1.58 (m, 2H), 1.49-1.47 (m, 1H), 1.16-1.14 (m, |
| | 4H), 1.02-1.0 (m, 1H), 0.81-0.69 (m, 6H). MS |
| | (ESI, m/e) [M + 1]⁺ 626.5. |
| 152 | ¹H NMR (400 MHz, DMSO-d₆) δ ppm: 10.06 |
| | (s, 1H), 8.43 (d, J = 8.0 Hz, 1H), 7.52-7.45 (m, |
| | 2H), 7.39-7.28 (m, 2H), 7.09-7.06 (m, 2H), 6.41 |
| | (s, 1H), 4.38-4.33 (m, 1H), 4.04-4.02 (m, 3H), |
| | 3.54-3.43 (m, 2H), 3.11-3.05 (m, 2H), 3.00-2.95 |
| | (m, 2H), 2.90-2.82 (m, 2H), 2.56 (d, J = 8.0 Hz, |
| | 3H), 2.03-1.61 (m, 5H), 1.47-1.34 (m, 2H), |
| | 1.23-1.11 (m, 2H), 1.02-0.93 (m, 2H), 0.88-0.82 |
| | (m, 6H), 0.42-0.38 (m, 2H), 0.22-0.17 (m, 2H). |
| | MS (ESI, m/e) [M + 1]⁺ 604.7 |
| 153 | ¹H NMR (400 MHz, DMSO-d₆) δ ppm: 10.07 (s, |
| | 1H), 8.70 (d, J = 8.0 Hz, 1H), 8.37 (s, 1H), 8.19 |
| | (d, J = 7.8 Hz, 1H), 8.01 (d, J = 7.8 Hz, 1H), |
| | 7.69 (t, J = 7.8 Hz, 1H), 7.48 (d, J = 33.8 Hz, |
| | 1H), 7.42-7.25 (m, 2H), 7.07 (d, J = 8.0 Hz, |
| | 1H), 6.41 (s, 1H), 4.43 (s, 1H), 3.53-3.46 (m, |
| | 2H), 3.13-2.99 (m, 4H), 2.85 (s, 2H), 2.56 (d, J = |
| | 4.5 Hz, 3H), 1.95-1.54 (m, 6H), 1.28-1.11 (m, |
| | 5H), 0.39 (s, 2H). 0.20-0.16 (m, 2H). MS (ESI, |
| | m/e) [M + 1]⁺ 583.5 |
| 154 | ¹H NMR (400 MHz, DMSO-d₆) δ ppm: 10.05 (s, |
| | 1H), 8.43 (d, J = 8.2 Hz, 1H), 7.53-7.43 (m, |
| | 2H), 7.40-7.27 (m, 2H), 7.07 (d, J = 8.2 Hz, |
| | 1H), 7.01 (s, 1H), 6.41 (s, 1H), 4.52-4.47 (m, |
| | 2H), 4.38 (t, J = 8.6 Hz, 1H), 3.66-3.58 (m, 1H), |
| | 3.52-3.48 (m, 2H), 3.2-2.91 (m, 5H), 2.86 (q, J = |
| | 11.8 Hz, 2H), 2.56 (d, J = 4.6 Hz, 3H), 1.89- |
| | 1.52 (m, 6H), 1.22-0.94 (m, 6H), 0.39 (s, 2H), |
| | 0.18 (q, J = 9.2 Hz, 2H). MS (ESI, m/e) [M + 1]⁺ |
| | 576.5 |
| 155 | ¹H NMR (400 MHz, DMSO-d₆) δ ppm: 10.08 |
| | (s, 1H), 8.44 (d, J = 8.4 Hz, 1H), 7.46-7.41 (m, |
| | 3H), 7.11-7.06 (m, 2H), 6.97 (s, 1H), 6.80 (d, J = |
| | 8.8 Hz, 1H), 4.39-4.35 (m, 1H), 4.02 (s, 3H), |
| | 3.52-3.43 (m, 2H), 3.39-3.36 (m, 1H), 3.16-3.09 |
| | (m, 1H), 2.94-2.91 (m, 1H), 1.99-1.96 (m, 5H), |
| | 1.88-1.86 (m, 9H), 1.70 (s, 2H), 1.61-1.56 (m, |
| | 9H), 1.15 (s, 3H), 0.85-0.76 (m, 6H). MS (ESI, |
| | m/e) [M + 1]⁺ 684.9. |
| 156 | ¹H NMR (400 MHz, DMSO-d₆) δ ppm: 10.08 |
| | (s, 1H), 8.47 (d, J = 8.0 Hz, 1H), 7.50-7.33 (m, |
| | 3H), 7.12-7.06 (m, 2H), 6.18 (s, 1H), 4.42-4.35 |
| | (m, 1H), 4.03 (s, 3H), 3.45-3.36 (m, 2H), 3.25- |
| | 3.07 (m, 9H), 2.95-2.82 (m, 2H), 1.85-2.56 (m, |
| | 6H), 1.22-0.88 (m, 5H), 0.58-0.44 (m, 4H). MS |
| | (ESI, m/e) [M + 1]⁺ 549.9. |
| 157 | ¹H NMR (400 MHz, DMSO-d₆) δ ppm: 10.05 (s, |
| | 1H), 8.46 (d, J = 8.2 Hz, 1H), 7.48 (d, J = 33.0 |
| | Hz, 1H), 7.33 (dd, J = 18.6, 13.6 Hz, 2H), 7.08 |
| | (d, J = 8.1 Hz, 1H), 6.92 (d, J = 4.2 Hz, 1H), |
| | 6.64 (d, J = 4.1 Hz, 1H), 6.43 (s, 1H), 4.38 (t, J = |
| | 8.4 Hz, 1H), 3.61-3.40 (m, 3H), 3.13-2.99 (m, |
| | 9H), 2.85 (d, J = 7.2 Hz, 2H), 2.56 (d, J = 4.4 |
| | Hz, 3H), 1.88-1.50 (m, 6H), 1.07-0.81 (m, 5H), |
| | 0.39 (s, 2H), 0.17 (d, J = 13.4 Hz, 2H). MS (ESI, |
| | m/e) [M + 1]⁺ 612.5 |
| 158 | ¹H NMR (400 MHz, DMSO-d₆) δ ppm: 10.11 |
| | (s, 1H), 9.11 (d, J = 8.0 Hz, 1H), 8.15 (s, 1H), |
| | 8.06 (d, J = 8.0 Hz, 1H), 7.96 (d, J = 8.0 Hz, |
| | 1H), 7.78-7.74 (m, 1H), 7.34 (d, J = 8.0 Hz, |
| | 1H), 7.25-7.21 (m, 1H), 7.07 (d, J = 8.0 Hz, |
| | 1H), 6.65 (s, 1H), 6.42 (s, 1H), 4.26-4.22 (m, |
| | 1H), 3.54-3.46 (m, 2H), 3.10-2.99 (m, 2H), |
| | 2.90-2.82 (m, 2H), 2.56 (d, J = 4.0 Hz, 3H), |
| | 1.68-1.58 (m, 4H), 1.52-1.43 (m, 4H), 0.40-0.38 |
| | (m, 2H), 0.20-0.15 (m, 2H). MS (ESI, m/e) |
| | [M + 1]⁺ 633.7 |
| 159 | ¹H NMR (400 MHz, DMSO-d₆) δ ppm: 10.06 |
| | (s, 1H), 8.41 (d, J = 8.0 Hz, 1H), 7.55-7.28 (m, |
| | 3H), 7.22-7.06 (m, 3H), 6.17 (s, 1H), 4.58-4.52 |
| | (m, 1H), 4.01 (s, 3H), 3.65-3.49 (m, 2H), 3.25- |
| | 3.07 (m, 9H), 2.85-2.70 (m, 2H), 0.92-0.78 (m, |
| | 2H), 0.58-0.14 (m, 12H). MS (ESI, m/e) |
| | [M + 1]⁺ 561.8. |
| 160 | ¹H NMR (400 MHz, DMSO-d₆) δ ppm: 10.04 |
| | (s, 1H), 8.40 (d, J = 8.0 Hz, 1H), 7.55-7.28 (m, |
| | 3H), 7.10 (d, J = 8.0 Hz, 1H), 6.92 (s, 1H), 6.18 |
| | (s, 1H), 5.55-5.42 (m, 1H), 4.88-4.72 (m, 1H), |
| | 3.65-3.49 (m, 2H), 3.25-3.07 (m, 9H), 2.85-2.70 |
| | (m, 2H), 1.48-1.30 (m, 6H), 0.92-0.78 (m, 2H), |
| | 0.58-0.14 (m, 12H). MS (ESI, m/e) [M + 1]⁺ |
| | 589.7. |
| 161 | ¹H NMR (400 MHz, DMSO-d₆) δ ppm: 10.09 |
| | (s, 1H), 8.46 (d, J = 8.2 Hz, 1H), 7.53 (d, J = 9.6 |
| | Hz, 1H), 7.46 (d, J = 1.8 Hz, 1H), 7.41-7.32 (m, |
| | 1H), 7.12 (d, J = 8.2 Hz, 1H), 7.06 (s, 1H), 6.99 |
| | (t, J = 7.8 Hz, 2H), 6.49-6.44 (m, 3H), 6.32 (s, |
| | 1H), 5.86 (t, J = 6.2 Hz, 1H), 4.39 (t, J = 8.6 Hz, |
| | 1H), 4.03 (s, 3H), 3.41-3.19 (m, 4H), 3.16-3.06 |
| | (m, 4H), 2.89-2.74 (m, 2H), 1.88-1.81 (m, 2H), |
| | 1.72-1.70 (m, 2H), 1.63-1.57 (m, 2H), 1.23-1.11 |
| | (m, 4H), 1.04-0.97 (m, 1H), 0.41-0.34 (m, 4H). |
| | MS (ESI, m/e) [M + 1]⁺ 611.0. |
| 162 | ¹H NMR (400 MHz, DMSO-d₆) δ ppm: 10.09 |
| | (s, 1H), 8.46 (d, J = 8.0 Hz, 1H), 7.58-7.54 (m, |
| | 1H), 7.43-7.41 (m, 2H), 7.18-7.02 (m, 2H), 6.64 |
| | (s, 1H), 4.57 (s, 1H), 4.39-4.37 (m, 1H), 4.03 (s, |
| | 3H), 3.23 (s, 2H), 2.98-2.90 (m, 5H), 1.84-1.82 |
| | (m, 2H), 1.70 (s, 2H), 1.58-1.53 (m, 3H), 1.15 |
| | (s, 3H), 0.88-0.70 (m, 7H). MS (ESI, m/e) |
| | [M + 1]⁺ 507.5. |
| 163 | ¹H NMR (400 MHz, DMSO-d₆) δ ppm: 10.12 |
| | (s, 1H), 8.51-8.42 (m, 2H), 8.27 (s, 1H), 7.66- |
| | 7.64 (m, 2H), 7.46-7.43 (m, 2H), 7.36-7.34 (m, |
| | 2H), 7.24-7.18 (m, 1H), 7.14 (d, J = 8.0 Hz, |
| | 1H), 7.06 (s, 1H), 6.91 (s, 1H), 4.44-4.27 (m, |
| | 3H), 4.01 (s, 3H), 3.66-3.48 (m, 2H), 3.22-3.18 |
| | (m, 2H), 2.96-2.93 (m, 1H), 1.83 (s, 2H), 1.68- |
| | 1.63 (m, 5H), 1.47-1.43 (m, 1H), 1.15 (s, 4H), |
| | 1.00 (s, 1H), 0.78-0.70 (m, 6H). MS (ESI, m/e) |
| | [M + 1]⁺ 642.2. |
| 164 | ¹H NMR (400 MHz, DMSO-d₆) δ ppm: 9.96 (s, |
| | 1H), 8.34 (d, J = 4.0 Hz, 1H), 7.34-7.25 (m, |
| | 2H), 7.20-7.09 (m, 2H), 6.89-6.87 (m, 2H), 6.23 |
| | (s, 1H), 4.22-4.18 (m, 1H), 3.84-3.83 (m, 3H), |
| | 3.34-3.23 (m, 2H), 2.91-2.85 (m, 2H), 2.83-2.75 |
| | (m, 2H), 2.69-2.63 (m, 2H), 2.36-2.34 (m, 3H), |
| | 2.07-1.98 (m, 1H), 1.81-1.65 (m, 4H), 1.51-1.48 |
| | (m, 1H), 1.09-0.97 (m, 4H), 0.21-0.15 (m, 2H), |
| | 0.04-0.00 (m, 2H). MS (ESI, m/e) [M + 1]⁺ 630.6 |
| 165 | ¹H NMR (400 MHz, DMSO-d₆) δ ppm: 10.06 |
| | (s, 1H), 8.45 (d, J = 8.0 Hz, 1H), 7.48-7.45 (m, |
| | 2H), 7.34 (d, J = 8.0 Hz, 1H), 7.09 (d, J = 8.0 |
| | Hz, 1H), 7.05 (d, J = 4.0 Hz, 1H), 6.18 (s, 1H), |
| | 4.37-4.33 (m, 1H), 4.02 (s, 3H), 3.41 (s, 2H), |
| | 3.22-3.18 (m, 6H), 3.12-3.03 (m, 2H), 2.89-2.82 |
| | (m, 2H), 2.03-1.97 (m, 1H), 1.88-1.74 (m, 4H), |
| | 1.61-1.58 (m, 1H), 1.23-1.11 (m, 5H), 1.06-0.97 |
| | (m, 2H), 0.86-0.82 (m, 3H), 0.56-0.48 (m, 4H). |
| | MS (ESI, m/e) [M + 1]⁺ 577.7 |
| 166 | ¹H NMR (400 MHz, DMSO-d₆) δ ppm: 10.07 (s, |
| | 1H), 8.40 (d, J = 8.8 Hz, 1H), 7.55-7.45 (m, |
| | 3H), 7.41-7.30 (m, 1H), 7.11 (d, J = 8.4 Hz, 1H), |
| | 7.05 (s, 1H), 6.71 (s, 1H), 4.79 (t, J = 9.0 Hz, |
| | 1H), 4.04 (s, 3H), 3.58-3.51 (m, 2H), 3.47-3.37 |
| | (m, 2H), 3.31-3.21 (m, 2H), 2.91-2.86 (m, 1H), |
| | 2.58 (d, J = 3.0 Hz, 3H), 2.06-1.92 (m, 1H), |
| | 1.41-1.31 (m, 3H), 0.87-0.76 (m, 5H), 0.42-0.19 |
| | (m, 8H). MS (ESI, m/e) [M + 1]⁺ 562.5 |
| 167 | ¹H NMR (400 MHz, DMSO-d₆) δ ppm: 10.01, |
| | (s, 1H), 8.02 (s, 1H), 7.59 (d, J = 4.4 Hz, 1H), |
| | 7.47 (s, 1H), 7.38-7.25 (m, 2H), 7.09 (d, J = 8.2 |
| | Hz, 1H), 6.43 (s, 1H), 4.47 (s, 1H), 3.92 (s, 2H), |
| | 3.58-3.38 (m, 2H), 3 .. 18-3.11 (m, 5H), 2.86 (s, |
| | 2H), 2.55 (d, J = 4.8 Hz, 3H), 1.81-1.52 (m, |
| | 6H), 1.28-1.12 m, 6H), 0.40 (s, 2H), 0.21-0.17 |
| | (m, 2H). MS (ESI, m/e) [M + 1]⁺ 580.5 |
| 168 | ¹H NMR (400 MHz, DMSO-d₆) δ ppm: 10.09 |
| | (s, 1H), 8.48 (d, J = 8.2 Hz, 1H), 7.48 (t, J = |
| | 16.4 Hz, 2H), 7.41-7.25 (m, 2H), 7.07 (d, J = |
| | 12.4 Hz, 2H), 6.43 (s, 1H), 4.37 (t, J = 8.4 Hz, |
| | 1H), 4.02 (s, 3H), 3.56-3.40 (m, 3H), 3.11-2.95 |
| | (m, 4H), 2.91-2.80 (m, 2H), 2.56 (d, J = 4.4 Hz, |
| | 3H), 2.23-2.09 (m, 2H), 1.87-1.78 (m, 4H), |
| | 1.61-1.52 (m, 2H), 1.22-1.16 (m, 1H), 1.11-0.98 |
| | (m, 2H), 0.44-0.36 (m, 2H), 0.21-0.13 (m, 2H). |
| | MS (ESI, m/e) [M + 1]⁺ 644.5. |
| 169 | ¹H NMR (400 MHz, DMSO-d₆) δ ppm: 12.20- |
| | 11.97 (m, 1H), 10.07 (s, 1H), 8.42 (d, J = 8.2 |
| | Hz, 1H), 7.54-7.50 (m, 4H), 7.35-7.30 (m, 1H), |
| | 7.16-7.13 (m, 3H), 7.04 (s, 1H), 6.77 (s, 1H), |
| | 4.38-4.36 (m, 1H), 4.03-3.84 (m, 5H), 3.64-3.52 |
| | (m, 3H), 3.25-3.11 (m, 2H), 1.82 (s, 2H), 1.65- |
| | 1.58 (m, 4H), 1.45-1.44 (m, 1H), 1.18-0.93 (m, |
| | 5H), 0.76-0.72 (m, 6H). MS (ESI, m/e) [M + 1]⁺ |
| | 623.6. |
| 171 | ¹H NMR (400 MHz, DMSO-d₆) δ ppm: 10.11 |
| | (s, 1H), 8.50 (d, J = 8.0 Hz, 1H), 7.54-7.45 (m, |
| | 2H), 7.40-7.29 (m, 2H), 7.10-7.06 (m, 2H), 6.43 |
| | (s, 1H), 4.38-4.33 (m, 1H), 4.03 (s, 3H), 3.53- |
| | 3.42 (m, 2H), 3.11-2.95 (m, 2H), 2.89-2.82 (m, |
| | 2H), 2.56 (d, J = 4.0 Hz, 3H), 1.88-1.75 (m, |
| | 4H), 1.61-1.57 (m, 1H), 1.23-1.11 (m, 5H), |
| | 1.06-0.97 (m, 2H), 0.90-0.77 (m, 3H), 0.43-0.36 |
| | (m, 2H), 0.21-0.15 (m, 2H). MS (ESI, m/e) |
| | [M + 1]⁺ 590.5 |
| 172 | ¹H NMR (400 MHz, DMSO-d₆) δ ppm: 10.12 |
| | (s, 1H), 8.49 (d, J = 8.2 Hz, 1H), 7.54 (d, J = |
| | 10.2 Hz, 1H), 7.45 (d, J = 1.4 Hz, 1H), 7.37 (dd, |
| | J = 13.2, 8.4 Hz, 1H), 7.12 (d, J = 8.2 Hz, 1H), |
| | 7.06 (s, 1H), 6.95 (dd, J = 12.2, 7.8 Hz, 1H), |
| | 6.86 (t, J = 7.8 Hz, 1H), 6.58 (t, J = 8.6 Hz, 1H), |
| | 6.49-6.44 (m, 2H), 5.99 (s, 1H), 4.38 (t, J = 8.4 |
| | Hz, 1H), 4.02 (s, 3H), 3.44-3.40 (m, 2H), 3.26- |
| | 3.07 (m, 6H), 2.90-2.78 (m, 2H), 1.88-1.80 (m, |
| | 2H), 1.72-1.69 (m, 2H), 1.62-1.55 (m, 2H), |
| | 1.20-1.12 (m, 4H), 1.05-0.92 (m, 1H), 0.44-0.38 |
| | (m, 4H). MS (ESI, m/e) [M + 1]⁺ 628.5. |
| 173 | ¹H NMR (400 MHz, DMSO-d₆) δ ppm: 10.10 |
| | (s, 1H), 8.47 (d, J = 8.4 Hz, 1H), 7.52 (d, J = |
| | 10.4 Hz, 1H), 7.46 (s, 1H), 7.41-7.33 (m, 1H), |
| | 7.12 (d, J = 8.0 Hz, 1H), 7.06 (s, 1H), 6.75 (d, |
| | J = 7.8 Hz, 1H), 6.66 (t, J = 7.4 Hz, 1H), 6.48 (t, |
| | J = 7.6 Hz, 1H), 6.41 (s, 1H), 6.35 (d, J = 7.8Hz, |
| | 1H), 5.33 (d, J = 4.6 Hz, 1H), 4.38 (t, J = 8.6 |
| | Hz, 1H), 4.02 (s, 3H), 3.73 (s, 3H), 3.20-3.12 |
| | (m, 6H), 2.87 (s, 2H), 2.04-1.94 (m, 2H), 1.86- |
| | 1.80 (m, 2H), 1.72-1.69 (m, 2H), 1.62-1.59 (m, |
| | 2H), 1.17-1.14 (s, 4H), 0.85 (t, J = 6.0 Hz, 1H), |
| | 0.49 (d, J = 9.2 Hz, 4H). MS (ESI, m/e) [M + 1]⁺ |
| | 640.5. |
| 174 | ¹H NMR (400 MHz, DMSO-d₆) δ ppm: 10.10 (s, |
| | 1H), 8.46 (d, J = 8.2 Hz, 1H), 8.23 (s, 1H), 7.54 |
| | (d,J = 6.8 Hz, 1H), 7.46 (s, 1H), 7.37 (d, J = 4.6 |
| | Hz, 1H), 7.11 (d, J = 8.0 Hz, 1H), 7.06 (s, 1H), |
| | 6.48 (s, 1H), 4.38 (d, J = 4.2 Hz, 1H), 4.02 (s, |
| | 3H), 3.55-3.44 (m, 2H), 3.25 (s, 1H), 3.11-3.07 |
| | (m, 2H), 3.02 (s, 1H), 2.65 (d, J = 14.8 Hz, 2H), |
| | 2.55 (d, J = 4.4 Hz, 3H), 2.37-2.24 (m, 2H), |
| | 2.20-2.01 (m, 2H), 1.83 (s, 2H), 1.69 (s, 2H), |
| | 1.59 (d, J = 18.8 Hz, 2H), 1.15 (s, 4H), 1.05- |
| | 0.98 (m, 1H). MS (ESI) m/e [M + 1]⁺ 612.5. |
| 175 | ¹H NMR (400 MHz, DMSO-d₆) δ ppm: 10.10 |
| | (s, 1H), 8.47 (d, J = 8.2 Hz, 1H), 8.03 (s, 1H), |
| | 7.60-7.20 (m, 5H), 7.17-7.02 (m, 4H), 6.59 (s, |
| | 1H), 4.43-4.26 (m, 3H), 4.03 (s, 3H), 3.65-3.54 |
| | (m, 2H), 3.23-2.96 (m, 4H), 2.89-2.85 (m, 2H), |
| | 1.83-1.82 (m, 2H), 1.70 (s, 2H), 1.59-1.56 (m, |
| | 2H), 1.15 (s, 4H), 0.39 (s, 2H), 0.21-0.18 (m, |
| | 2H). MS (ESI, m/e) [M + 1]⁺ 656.5. |
| 176 | MS (ESI, m/e) [M + 1]⁺ 636.7 |
| 177 | ¹H NMR (400 MHz, DMSO-d₆) δ ppm: 10.10 |
| | (s, 1H), 8.42 (d, J = 8.8 Hz, 1H), 7.59 (s, 1H), |
| | 7.52-7.46 (m, 2H), 7.34 (d, J = 8.8 Hz, 1H), 7.17 |
| | (d, J = 8.4 Hz, 1H), 6.93 (d, J = 1.8 Hz, 1H), |
| | 5.48-5.34 (m, 1H), 4.82-4.78 (m, 1H), 4.66-4.54 |
| | (m, 1H), 4.31 (s, 1H), 3.50-3.47 (m, 1H), 3.17- |
| | 3.15 (m, 1H), 3.03-2.98 (m, 2H), 2.88-2.80 (m, |
| | 2H), 1.39-1.34 (m, 6H), 0.93-0.68 (m, 3H), |
| | 0.45-0.07 (m, 8H). MS (ESI, m/e) [M + 1]⁺ |
| | 573.6. |
| 178 | ¹H NMR (400 MHz, DMSO-d₆) δ ppm: 10.22 |
| | (s, 1H), 9.23 (d, J = 8.0 Hz, 1H), 7.60 (s, 1H), |
| | 7.48 (s, 1H), 7.33 (d, J = 8.0 Hz, 1H), 7.18 (d, |
| | J = 8.0 Hz, 1H), 4.59-4.56 (m, 2H), 4.32 (s, 1H), |
| | 3.50-3.46 (m, 1H), 3.16-3.12 (m, 1H), 3.04-3.0 |
| | (m, 2H), 2.95-2.86 (m, 3H), 2.47 (s, 3H), 2.04 |
| | (s, 3H), 1.93-1.64 (m, 4H), 1.52-1.27 (m, 2H). |
| | MS (ESI, m/e) [M + 1]⁺ 571.5. |
| 179 | ¹H NMR (400 MHz, DMSO-d₆) δ ppm: 10.11 |
| | (s, 1H), 8.48 (d, J = 8.2 Hz, 1H), 7.60-7.23 (m, |
| | 4H), 7.11 (d, J = 8.2 Hz, 1H), 7.07 (s, 1H), 6.68 |
| | (s, 1H), 4.38 (t, J = 8.4 Hz, 1H), 4.03 (s, 3H), |
| | 3.69-3.46 (m, 3H), 3.27 (d, J = 12.2 Hz, 1H), |
| | 3.15-3.08 (m, 3H), 2.91 (d, J = 9.8 Hz, 1H), 2.55 |
| | (d, J = 4.4 Hz, 3H), 1.86-1.81 (m, 2H), 1.72- |
| | 1.69 (m, 2H), 1.62-1.54 (m, 2H), 1.18-1.12 (m, |
| | 4H), 1.06-0.94 (m, 1H), 0.88 (d, J = 6.4 Hz, |
| | 3H). MS (ESI, m/e) [M + 1]⁺ 618.5. |
| 180 | ¹H NMR (400 MHz, DMSO-d₆) δ ppm: 10.14 (s, |
| | 1H), 9.68 (d, J = 7.0 Hz, 1H), 8.76 (d, J = 8.4 |
| | Hz, 1H), 8.72 (s, 1H), 8.17 (d, J = 7.2 Hz, 1H), |
| | 7.50 (s,1H), 7.34-7.24 (m, 3H), 7.08 (d, J = 8.0 |
| | Hz, 1H), 6.43 (s, 1H), 4.48 (t, J = 9.0 Hz,1H), |
| | 3.56-3.46 (m, 2H), 3.12-2.91 (m, 4H), 2.85 (s, |
| | 2H), 2.55 (d, J = 4.6 Hz, 3H), 1.95-1.55 (m, |
| | 6H), 1.29-0.93 (m, 5H), 0.39 (s, 2H), 0.24-0.07 |
| | (m, 2H). (ESI, m/e) [M + 1]⁺ 623.6 |
| 181 | ¹H NMR (400 MHz, DMSO-d₆) δ ppm: 10.16 |
| | (s, 1H), 9.13-9.11 (m, 1H), 7.52-7.43 (m, 1H), |
| | 7.40-7.26 (m, 2H), 7.09 (d, J = 8.0 Hz, 1H), 6.44 |
| | (s, 1H), 4.49-4.45 (m, 1H), 3.54-3.43 (m, 2H), |
| | 3.11-3.05 (m, 2H), 3.01-2.98 (m, 2H), 2.89-2.84 |
| | (m, 2H), 2.56 (d, J = 4.0 Hz, 3H), 2.48 (s, 3H), |
| | 2.27-2.15 (m,1H), 2.01-1.84 (m, 4H), 1.74-1.68 |
| | (m, 1H), 1.23-1.16 (m, 4H), 0.43-0.36 (m, 2H), |
| | 0.21-0.15 (m, 2H). MS (ESI, m/e) [M + 1]⁺ 632.6 |
| 182 | ¹H NMR (400 MHz, DMSO-d₆) δ ppm: 10.09 |
| | (s, 1H), 8.47 (d, J = 7.8 Hz, 1H), 7.96 (s, 1H), |
| | 7.53 (s, 1H), 7.46-7.43 (m, 1H), 7.33 (d, J = 8.8 |
| | Hz, 1H), 7.09-7.06 (m, 2H), 6.56 (s, 1H), 4.39- |
| | 4.35 (m, 1H), 4.03 (s, 3H), 3.50-3.45 (m, 2H), |
| | 3.12-2.78 (m, 8H), 2.35 (s, 1H), 1.94 (s, 6H), |
| | 1.83 (s, 2H), 1.72-1.70 (m, 2H), 1.63-1.60 (m, |
| | 2H), 1.16-1.10 (s, 3H), 0.42 (s, 2H), 0.25-0.18 |
| | (m, 2H). MS (ESI, m/e) [M + 1]⁺ 614.5. |
| 183 | ¹H NMR (400 MHz, DMSO-d₆) δ ppm: 9.99 (s, |
| | 1H), 8.91 (d, J = 8.0 Hz, 1H), 7.44 (s, 1H), 7.35 |
| | (s, 1H), 7.18 (d, J = 12.0 Hz, 1H), 7.05 (d, J = |
| | 12.0 Hz, 1H), 4.74-4.70 (m, 1H), 4.48-4.45 (m, |
| | 1H), 4.24-4.14 (m, 1H), 3.38-3.33 (m, 1H), |
| | 3.04-3.00 (m, 1H), 2.91-2.85 (m, 2H), 2.79-2.73 |
| | (m, 1H), 2.70-2.65 (m,1H), 2.35 (s, 3H), 0.74- |
| | 0.58 (m, 3H), 0.35-0.30 (m, 1H), 0.27-0.23 (m, |
| | 1H), 0.21-0.01 (m, 6H). MS (ESI, m/e) [M + 1]⁺ |
| | 547.4 |
| 184 | ¹H NMR (400 MHz, DMSO-d₆) δ ppm: 11.94 |
| | (s, 1H), 10.17 (s, 1H), 9.22 (d, J = 8.0 Hz, 1H), |
| | 7.55-7.22 (m, 4H), 7.15 -7.04 (m, 3H), 6.41 (s, |
| | 1H), 4.56-4.48 (m, 1H), 4.19-4.07 (m, 2H), |
| | 3.49-3.36 (m, 2H), 3.30-3.22 (m, 2H), 2.96-2.87 |
| | (m, 2H), 2.48 (s, 3H), 2.10-1.60 (m, 7H), 1.50- |
| | 1.30 (m, 2H), 0.48-0.34 (m, 4H). MS (ESI, m/e) |
| | [M + 1]⁺ 659.5. |
| 185 | ¹H NMR (400 MHz, DMSO-d₆) δ ppm: 10.19 |
| | (s, 1H), 9.24 (d, J = 8.0 Hz, 1H), 7.55-7.26 (m, |
| | 3H), 7.11 (d, J = 8.0 Hz, 1H), 6.67 (s, 1H), 4.58- |
| | 4.50 (m, 1H), 3.58 (t, J = 17.2 Hz, 2H), 3.18- |
| | 2.97 (m, 5H), 2.93-2.83 (m, 3H), 2.48 (s, 3H), |
| | 2.10-1.65 (m, 8H), 1.59-1.30 (m, 7H), 1.19-0.95 |
| | (m, 6H), 0.50-0.28 (m, 4H). MS (ESI, m/e) |
| | [M + 1]⁺ 710.6. |
| 186 | ¹H NMR (400 MHz, DMSO-d₆) δ ppm: 10.22 |
| | (d, J = 11.8 Hz, 1H), 8.66 (d, J = 8.2 Hz, 1H), |
| | 7.59-7.38 (m, 3H), 7.37 (d, J = 25.8 Hz, 1H), |
| | 7.20-7.04 (m, 5H), 6.78 (s, 1H), 4.55 (t, J = 8.8 |
| | Hz, 1H), 4.04 (s, 3H), 3.58-3.46 (m, 2H), 3.33- |
| | 3.29 (m, 3H), 3.23-3.08 (m, 2H), 2.91-2.79 (m, |
| | 2H), 2.79-2.69 (m, 2H), 2.57 (d, J = 4.2 Hz, |
| | 3H), 2.38-2.28 (m, 1H), 1.99-1.88 (m , 1H), |
| | 1.48-1.38 (m, 2H), 0.72 (dd, J = 19.4, 6.2 Hz, |
| | 6H). MS (ESI, m/e) [M + 1]⁺ 646.5 |
| 187 | ¹H NMR (400 MHz, DMSO-d₆) δ ppm: 12.32 |
| | (d, J = 12.6 Hz, 1H), 10.19 (s, 1H), 9.21 (d, J = |
| | 8.6 Hz, 1H), 7.59-7.49 (m, 1H), 7.42-7.22 (m, |
| | 2H), 7.15 (d, J = 8.6 Hz, 2H), 6.93 (s, 1H), 6.44 |
| | (s, 1H), 4.52 (t, J = 8.4 Hz, 1H), 4.14-4.00 (m, |
| | 2H), 3.56-3.49 (m, 2H) 3.27 (s, 2H), 2.87 (s, |
| | 2H), 2.46 (s, 3H), 2.05-2.01 (m, 3H), 1.85-1.61 |
| | (m, 4H), 1.43-1.28 (m, 2H), 0.44-0.39 (m, 4H). |
| | MS (ESI, m/e) [M + 1]⁺678.1 |
| 188 | ¹H NMR (400 MHz, DMSO-d₆) δ ppm: 12.05 |
| | (s, 1H), 10.20 (s, 1H), 9.24 (d, J = 8.0 Hz, 1H), |
| | 7.55-7.26 (m, 2H), 7.12 (d, J = 8.0 Hz, 1H), 6.45 |
| | (s, 1H), 4.58-4.50 (m, 1H), 3.49-3.36 (m, 2H), |
| | 3.20-3.12 (m, 2H), 3.06-2.97 (m, 2H), 2.86 (s, |
| | 2H), 2.48 (s, 3H), 2.10-1.65 (m, 7H), 1.50-1.30 |
| | (m, 2H), 0.50-0.24 (m, 4H). MS (ESI, m/e) |
| | [M + 1]⁺ 587.6. |
| 189 | ¹H NMR (400 MHz, DMSO-d₆) δ ppm: 12.03 |
| | (s, 1H), 10.10 (s, 1H), 8.47 (d, J = 8.4 Hz, 1H), |
| | 7.54 (s, 1H), 7.45 (s, 1H), 7.39 (d, J = 7.4 Hz, |
| | OH), 7.30 (d, J = 8.0 Hz, 0.5H), 7.12-7.01 (m, |
| | 2H), 6.44 (s, 1H), 4.39-4.36 (m, 1H), 4.03 (s, |
| | 3H), 3.46-3.40 (m, 1H), 3.16-3.14 (m, 2H), |
| | 3.10-3.01 (m, 2H), 2.86-2.84 (m, 2H), 1.84-1.80 |
| | (m, 2H), 1.72-1.70 (m, 2H), 1.63-1.60 (m, 2H), |
| | 1.19-1.10 (m, 6H), 0.43 (s, 2H), 0.29-0.21 (m, |
| | 2H). MS (ESI, m/e) [M + 1]⁺ 549.5. |
| 190 | ¹H NMR (400 MHz, DMSO-d₆) δ ppm: 10.19 |
| | (s, 1H), 9.22 (d, J = 8.2 Hz, 1H), 7.52-7.43 (m, |
| | 2H), 7.38 (d, J = 8.6 Hz, 0.5H), 7.28 (d, J = 8.2 |
| | Hz, 0.5H), 7.11 (d, J = 8.0 Hz, 1H), 6.59 (s, 1H), |
| | 4.56-4.51 (m, 1H), 4.19-4.06 (m, 1H), 3.57-3.43 |
| | (m, 2H), 3.10-3.01 (m, 5H), 2.89-2.86 (m, 3H), |
| | 2.47 (s, 2H), 2.34-2.30 (m, 1H), 2.08-1.97 (m, |
| | 5H), 1.87 (s, 1H), 1.70-1.65 (m, 2H), 1.49-1.20 |
| | (m, 3H), 0.42 (s, 2H), 0.40 -0.20 (m, 2H). MS |
| | (ESI, m/e) [M + 1]⁺ 690.6. |
| 191 | ¹H NMR (400 MHz, DMSO-d₆) δ ppm: 10.19 |
| | (s, 1H), 9.23 (d, J = 8.4 Hz, 1H), 7.55-7.32 (m, |
| | 3H), 7.10-7.05 (m, 1H), 6.62 (s, 1H), 4.56-4.51 |
| | (m, 1H), 3.56-3.51 (s, 3H), 3.27-2.95 (m, 4H), |
| | 2.89-2.85 (m, 1H), 2.47 (s, 1H), 1.95-1.67 (m, |
| | 14H), 1.55-1.42 (m, 4H), 0.45-0.43 (m, 2H), |
| | 0.34-0.25 (m, 2H). MS (ESI, m/e) [M + 1]⁺ |
| | 704.6. |
| 192 | ¹H NMR (400 MHz, DMSO-d₆) δ ppm: 12.49 |
| | (s, 1H), 10.19 (s, 1H), 9.21 (d, J = 8.0 Hz, 1H), |
| | 7.69 (d, J = 8.6 Hz, 1H), 7.57-7.50(m, 1H), 7.42 |
| | (d, J = 7.6 Hz, 1H), 7.39-7.23 (m, 2H), 7.15 (d, |
| | J = 8.2 Hz, 1H), 6.39 (s, 1H), 4.52 (t, J = 8.2 Hz, |
| | 1H), 4.14-4.02 (m, 2H), 3.45 (s, 2H), 2.86 (s, |
| | 2H), 2.69 (s, 2H), 2.46 (s, 3H), 2.00 (d, J = 7.6 |
| | Hz, 4H), 1.89-1.62 (m, 5H), 1.48-1.29 (m, 4H). |
| | MS (ESI) m/e [M + 1]⁺ 727.7. |
| 193 | ¹H NMR (400 MHz, DMSO-d₆) δ ppm: 10.19 |
| | (s, 1H), 8.54 (d, J = 8.0 Hz, 1H), 7.63-7.44 (m, |
| | 2H), 7.43-7.33 (m, 1H), 7.25 (s, 1H), 7.14 (d, J = |
| | 8.4 Hz, 1H), 7.07 (s, 1H), 4.36 (t, J = 8.6 Hz, |
| | 1H), 4.02 (s, 3H), 3.52-3.42 (m, 2H), 3.17-3.06 |
| | (m, 2H), 2.92-2.85 (m, 4H), 1.87-1.82 (m, 2H), |
| | 1.72-1.69 (m, 2H), 1.62-1.57 (m, 2H), 1.18-1.11 |
| | (m, 4H), 1.06-0.94 (m, 1H), 0.51 (d, J = 9.6 Hz, |
| | 4H). MS (ESI, m/e) [M + 1]⁺ 601.4. |
| 194 | ¹H NMR (400 MHz, DMSO-d₆) δ ppm: 10.15 |
| | (s, 1H), 8.52 (d, J = 8.2 Hz, 1H), 7.61-7.45 (m, |
| | 2H), 7.38 (dt, J = 15.8, 8.2 Hz, 1H), 7.13 (dd, J = |
| | 7.8, 4.4 Hz, 1H), 7.07 (s, 1H), 6.72 (d, J = 15.6 |
| | Hz, 1H), 4.69 (s, 1H), 4.38 (t, J = 8.6 Hz, 1H), |
| | 4.03 (s, 3H), 3.53-3.35 (m, 3H), 3.26-3.03 (m, |
| | 3H), 2.95-2.72 (m, 2H), 2.70-2.57 (m, 1H), |
| | 1.86-1.81 (m, 2H), 1.72-1.69 (m, 2H), 1.62-1.54 |
| | (m, 2H), 1.18-1.12 (m, 4H), 1.04-0.96 (m, 1H), |
| | 0.38-0.32 (m, 2H), 0.21-0.07 (m, 2H). MS (ESI, |
| | m/e) [M + 1]⁺ 603.4. |
| 195 | ¹H NMR (400 MHz, DMSO-d₆) δ ppm: 10.11 |
| | (s, 1H), 8.46 (d, J = 4.0 Hz, 1H), 7.62-7.58 (m, |
| | 1H), 7.49 (s, 2H), 7.38-7.33 (m, 1H), 7.17 (d, J = |
| | 8.0 Hz, 1H), 6.95 (s, 1H), 5.42-5.36 (m, 1H), |
| | 4.61-4.58 (m, 1H), 4.38-4.28 (m, 2H), 3.50-3.45 |
| | (m, 1H), 3.16-3.11 (m, 1H), 3.04-2.98 (m, 2H), |
| | 2.91-2.85 (m, 1H), 2.82-2.77 (m,1H), 1.89-1.77 |
| | (m, 2H), 1.77-1.67 (m, 2H), 1.62-1.55 (m, 2H), |
| | 1.37-1.33 (m, 6H), 1.23-1.15 (m, 4H), 1.02-0.97 |
| | (m, 1H). MS (ESI, m/e) [M + 1]⁺ 561.5 |
| 196 | ¹H NMR (400 MHz, DMSO-d₆) δ ppm: 10.22 |
| | (d, J = 12.6 Hz, 1H), 8.66 (d, J = 7.4 Hz, 1H), |
| | 7.46 (t, J = 8.8 Hz, 4H), 7.20-7.06 (m, 5H), 6.78 |
| | (s, 1H), 4.55 (s, 1H), 4.09-4.04 (m, 2H), 3.61- |
| | 3.45 (m, 2H), 3.24-3.02 (m, 4H), 2.88-2.78 (m, |
| | 5H), 2.57 (s, 3H), 2.45-2.35 (m, 2H), 2.05-1.99 |
| | (m, 1H), 1.49-1.42 (m, 2H), 0.79-0.66 (m, 6H). |
| | MS (ESI, m/e) [M + 1]⁺ 646.4 |
| 197 | ¹H NMR (400 MHz, DMSO-d₆) δ ppm: 10.11 |
| | (s, 1H), 8.43 (d, J = 8.0 Hz, 1H), 7.59-7.52 (m, |
| | 1H), 7.47 (s, 2H), 7.40-7.33 (m, 1H), 7.17 (d, J = |
| | 8.0 Hz, 1H), 7.05 (d, J = 4.0 Hz, 1H), 4.82- |
| | 4.78 (m, 1H), 4.61-4.58 (m, 1H), 4.38-4.28 (m, |
| | 1H), 4.01 (s, 3H), 3.51-3.46 (m, 1H), 3.16-2.98 |
| | (m, 3H), 2.94-2.78 (m, 2H), 0.87-0.73 (m, 3H), |
| | 0.46-0.43 (m, 1H), 0.39-0.34 (m, 2H), 0.30-0.26 |
| | (m, 1H), 0.22-0.18 (m, 3H), 0.11-0.07 (m, 1H). |
| | MS (ESI, m/e) [M + 1]⁺ 545.6 |
| 198 | ¹H NMR (400 MHz, DMSO-d₆) δ ppm: 12.48 |
| | (s, 1H), 10.06 (d, J = 5.6 Hz, 1H), 8.39 (d, J = |
| | 8.6 Hz, 1H), 7.69 (d, J = 7.8 Hz, 1H), 7.50-7.38 |
| | (m, 3H), 7.33-7.22 (m, 2H), 7.14 (d, J = 8.2 Hz, |
| | 1H), 6.91 (s, 1H), 6.38 (s, 1H), 5.41-5.36 (m, |
| | 1H), 4.76 (t, J = 8.2 Hz, 1H), 4.14-3.95 (m, 2H), |
| | 3.49-3.40 (m, 2H), 2.86 (s, 2H), 2.69 (s, 2H), |
| | 1.36 (d, J = 6.6 Hz, 3H), 1.33 (d, J = 6.6 Hz, |
| | 3H), 1.23 (s, 1H), 0.86-0.67 (m, 4H), 0.34 (s, |
| | 3H), 0.28 (d, J = 8.8 Hz, 1H), 0.18 (s, 3H), 0.07 |
| | (s, 1H). MS (ESI) m/e [M + 1]⁺ 729.5. |
| 199 | ¹H NMR (400 MHz, DMSO-d₆) δ ppm: 10.07 |
| | (s, 1H), 8.47-8.45(m, 1H), 7.57-7.54 (m, 2H), |
| | 7.34-7.29 (m, 1H), 7.09-7.05 (m, 2H), 6.34 (s, |
| | 1H), 4.35-4.30 (m, 1H), 4.03 (s, 1H), 2.89-2.85 |
| | (m, 6H), 2.05-2.01 (m, 2H), 1.91-1.63 (m, 4H), |
| | 1.49-1.20 (m, 3H), 0.45 (s, 4H). MS (ESI, m/e) |
| | [M + 1]⁺ 505.6. |
| 200 | ¹H NMR (400 MHz, DMSO-d₆) δ ppm: 10.12 |
| | (s, 1H), 9.45 (s, 1H), 8.47-8.45(m, 1H), 7.57- |
| | 7.54 (m, 2H), 7.48-7.39 (m, 2H), 7.35-7.30 (m, |
| | 3H), 6.86-6.84 (m, 3H), 6.64 (s, 1H), 4.35-4.30 |
| | (m, 1H), 4.03 (s, 1H),3.70-3.54 (m, 2H), 3.24- |
| | 3.03 (m, 5H), 2.89-2.85 (m, 2H), 2.05-2.01 (m, |
| | 3H), 1.91-1.63 (m, 4H), 1.49-1.20 (m, 3H), 0.45 |
| | (s, 2H), 0.30-0.20 (m, 2H). MS (ESI, m/e) |
| | [M + 1]⁺ 642.8. |
| 201 | ¹H NMR (400 MHz, DMSO-d₆) δ ppm: 10.26- |
| | 10.21 (m, 1H), 8.68 (d, J = 8.6 Hz, 1H), 7.6- |
| | 7.25 (m, 4H), 7.24 (d, J = 7.2 Hz, 1H), 7.18- |
| | 6.97 (m, 4H), 6.78 (s, 1H), 4.55 (s, 1H), 4.05 (s, |
| | 3H), 3.60-3.45 (m, 2H), 3.24-3.12 (m, 3H), 2.90 |
| | (s, 3H), 2.70 (d, J = 26.4 Hz,2H), 2.57 (d, J = |
| | 4.2 Hz, 3H), 2.35-2.32 (m, 2H), 2.05-2.02 (m, |
| | 1H), 1.62-1.33 (m, 2H), 0.77-0.52 (m, 6H). MS |
| | (ESI, m/e) [M + 1]⁺ 646.4 |
| 202 | MS (ESI) m/e [M + 1]⁺ 590.4. |
| 203 | ¹H NMR (400 MHz, DMSO-d₆) δ ppm: 10.22 |
| | (s, 1H), 9.47 (s, 1H), 9.24 (d, J = 8.0 Hz, 1H), |
| | 7.57 (s, 2H), 7.54 (s, 1H), 7.48-7.39 (m, 1H), |
| | 7.35-7.30 (m, 3H), 7.15 (d, J = 8.0 Hz, 1H), |
| | 6.86-6.84 (m, 1H), 6.64 (s, 1H), 4.55-4.52 (m, |
| | 1H), 3.70-3.54 (m, 2H), 3.24-3.03 (m, 5H), |
| | 2.89-2.85 (m, 2H), 2.47 (s, 3H), 2.05-2.01 (m, |
| | 3H), 1.91-1.63 (m, 4H), 1.49-1.20 (m, 4H), 0.45 |
| | (s, 2H), 0.30-0.20 (m, 2H). |
| 204 | ¹H NMR (400 MHz, DMSO-d₆) δ ppm: 10.16 |
| | (s, 1H), 8.75 (d, J = 8.2 Hz, 1H), 7.54-7.52 (m, |
| | 1H), 7.45-7.35(m, 6H), 7.13 (d, J = 8.2 Hz, 1H), |
| | 6.95 (s, 1H), 6.77 (s, 1H), 4.78 (s, 1H), 3.94 (s, |
| | 3H), 3.58-3.44 (m, 2H), 3.22-3.10 (m, 3H), |
| | 3.04-2.80 (m, 2H), 2.59-2.54 (m, 3H), 1.47-1.40 |
| | (m, 1H), 1.29-1.26 (m, 5H), 0.77-0.67 (m, 6H). |
| | MS (ESI, m/e) [M + 1]⁺ 606.5. |
| 205 | ¹H NMR (400 MHz, DMSO-d₆) δ ppm: 11.69 |
| | (s, 1H), 10.16 (s, 1H), 9.21 (d, J = 8.0 Hz, 1H), |
| | 7.69 (d, J = 7.6 Hz, 1H), 7.65-7.47 (m, 2H), |
| | 7.37-7.20 (m, 4H), 7.15-7.07 (m, 2H), 6.34 (s, |
| | 1H), 4.58-4.47 (m, 1H), 4.17-4.05 (m, 2H), |
| | 3.28-3.20 (m, 4H), 2.86 (s, 2H), 2.48 (s, 3H), |
| | 2.10-1.60 (m, 8H), 1.50-1.30 (m, 2H), 0.50-0.40 |
| | (m, 4H). MS (ESI, m/e) [M + 1]⁺ 685.7. |
| 206 | ¹H NMR (400 MHz, DMSO-d₆) δ ppm: 11.68 |
| | (s, 1H), 10.02 (s, 1H), 8.40 (d, J = 8.6 Hz, 1H) |
| | 7.64-7.52 (m, 2H), 7.50-7.35 (m, 4H), 7.30-7.14 |
| | (m, 2H), 6.91 (s, 1H), 6.33 (s, 1H), 4.77-4.74 |
| | (m, 1H), 4.10-4.04 (m, 1H), 3.65-3.40 (m, 4H), |
| | 2.91-2.84 (m, 2H), 1.40-1.33 (m, 6H), 0.89-0.70 |
| | (m, 3H), 0.45-0.11 (m, 12H). MS (ESI, m/e) |
| | [M + 1]⁺ 687.6 |
| 207 | ¹H NMR (400 MHz, DMSO-d₆) δ ppm: 10.18 |
| | (s, 1H), 9.32 (s, 1H), 7.52-7.25 (m, 2H), 7.12- |
| | 7.06 (m, 1H), 6.65 (s, 1H), 4.57-4.53 (m, 2H), |
| | 3.65-3.30 (m, 3H), 3.28-3.10 (m, 2H), 2.89-2.80 |
| | (m, 2H), 2.49-2.45 (m, 3H), 2.01-1.63 (m, 7H), |
| | 1.55-1.43 (m, 5H), 0.78-0.70 (m, 6H), 0.58-0.41 |
| | (m, 4H), MS (ESI, m/e) [M + 1]⁺ 657.7 |
| 208 | ¹H NMR (400 MHz, DMSO-d₆) δ ppm: 10.23- |
| | 10.18 (m, 1H), 9.33-9.29 (m, 1H), 7.57-7.42 (m, |
| | 3H), 7.12-7.01 (s, 4H), 4.57-4.53 (m, 2H), 4.32- |
| | 4.30 (m, 1H), 3.65-3.30 (m, 2H), 3.28-3.10 (m, |
| | 5H), 2.85-2.80 (m, 3H), 2.35-2.30 (m, 1H), |
| | 1.87-1.83 (m, 1H), 1.55-1.23 (m, 5H). MS (ESI, |
| | m/e) [M + 1]⁺ 617.3 |
| 209 | ¹H NMR (400 MHz, DMSO-d₆) δ ppm: 10.07 |
| | (s, 1H), 8.44 (d, J = 8.4 Hz, 1H), 7.85-7.80 (m, |
| | 1H), 7.58-7.50 (m, 2H), 7.32-7.28 (s, 1H), 7.11- |
| | 6.98 (m, 3H), 4.47-4.43 (m, 1H), 4.05 (s, 3H), |
| | 3.61-3.56 (m, 1H), 3.24-3.22 (m, 4H), 3.10-3.05 |
| | (m, 4H), 2.49-2.28 (m, 1H), 1.95-1.65 (m, 5H), |
| | 1.82-1.58 (m, 3H), 1.24-1.20 (m, 5H), 0.89-0.79 |
| | (m, 5H). MS (ESI, m/e) [M + 1]⁺ 506.4. |
| 210 | ¹H NMR (400 MHz, DMSO-d₆) δ ppm: 10.30 |
| | (s, 1H), 9.27 (d, J = 8.0 Hz, 1H), 7.64-7.55 (m, |
| | 1H), 7.48-7.37 (m, 1H), 7.24 (d, J = 8.4 Hz, |
| | 1H), 6.87 (s, 1H), 4.58-4.53 (m, 1H), 3.75-3.64 |
| | (m, 2H), 3.28-3.23 (m, 2H), 3.16 (s, 2H), 2.93 |
| | (s, 2H), 2.48 (s, 3H), 2.10-1.64 (m, 7H), 1.50- |
| | 1.30 (m, 2H), 0.60-0.52 (m, 4H). MS (ESI, m/e) |
| | [M + 1]⁺ 568.6. |
| 211 | ¹H NMR (400 MHz, DMSO-d₆) δ ppm: 10.05 |
| | (s, 1H), 9.03 (d, J = 8.8 Hz, 1H), 7.85 (s, 1H), |
| | 7.47-7.45 (m, 1H), 7.35-7.16 (m, 1H), 7.13 (d, |
| | J = 8.4 Hz, 1H), 4.85-4.82 (m, 1H), 3.23-2.55 |
| | (m, 7H), 2.48 (s, 3H), 2.47-2.38 (m, 1H), 1.89- |
| | 1.82 (m, 1H), 1.59-1.42 (m, 1H), 0.90-0.69 (m, |
| | 7H), 0.51- 0.11 (m, 8H). MS (ESI, m/e) [M + 1]⁺ |
| | 520.6. |
| 213 | ¹H NMR (400 MHz, DMSO-d₆) δ ppm: 10.15 |
| | (s, 1H), 9.07 (d, J = 8.0 Hz, 1H), 7.60-7.57 (m, |
| | 1H), 7.47 (s, 1H), 7.36-7.32 (m, 1H), 7.19-7.16 |
| | (m, 1H), 4.63-4.56 (m, 1H), 4.48-4.44 (m, 1H), |
| | 4.35-4.28 (m, 1H), 3.50-3.45 (m, 1H), 3.15-3.12 |
| | (m, 1H), 3.04-2.98 (m, 2H), 2.91-2.86 (m, 1H), |
| | 2.83-2.77 (m, 1H), 2.47 (s, 3H), 1.87-1.79 (m, |
| | 2H), 1.74-1.68 (m, 2H), 1.65-1.55 (m, 2H), |
| | 1.23-1.05 (m, 5H). MS (ESI, m/e) [M + 1]⁺ 535.3 |
| 214 | ¹H NMR (400 MHz, DMSO-d₆) δ ppm: 10.21 |
| | (s, 1H), 9.24 (d, J = 8.4 Hz, 1H), 9.06 (d, J = 4.4 |
| | Hz, 1H), 7.58-7.47 (m, 2H), 7.44-7.30 (m, 1H), |
| | 7.21-7.11 (m, 3H), 7.02 (d, J = 7.6 Hz, 1H), 6.78 |
| | (s, 1H), 4.58-4.53 (m, 1H), 3.75-3.62 (m, 2H), |
| | 3.28-3.20 (m, 2H), 3.10-3.00 (m, 2H), 2.87 (s, |
| | 2H), 2.48 (s, 3H), 2.20 (s, 3H), 2.10-1.64 (m, |
| | 7H), 1.50-1.30 (m, 2H), 0.50-0.34 (m, 4H). MS |
| | (ESI, m/e) [M + 1]⁺ 676.4. |
| 215 | ¹H NMR (400 MHz, DMSO-d₆) δ ppm: 10.68 |
| | (s, 1H), 10.22 (s, 1H), 9.32 (s, 1H), 8.52 (s, 1H), |
| | 7.58-7.25 (m, 3H), 7.12-7.06 (m, 1H), 6.65 (s, |
| | 1H), 4.57-4.53 (m, 1H), 3.65-3.30 (m, 3H), |
| | 3.28-3.10 (m, 3H), 2.89-2.80 (m, 2H), 2.49-2.45 |
| | (m, 3H), 2.01-1.63 (m, 9H), 1.55-1.23 (m, 3H), |
| | 0.48-0.30 (m, 2H), 0.28-0.21 (m, 2H). MS (ESI, |
| | m/e) [M + 1]⁺ 669.4 |
| 216 | ¹H NMR (400 MHz, DMSO-d₆) δ ppm: 10.13 |
| | (s, 1H), 9.44 (s, 1H), 9.02 (d, J = 8.6 Hz, 1H) |
| | 7.64-7.52 (m, 2H), 7.50-7.35 (m, 4H), 7.30-7.14 |
| | (m, 1H), 6.85 (s, 1H), 6.61 (s, 1H), 4.87-4.83 |
| | (m, 1H), 3.65-3.40 (m, 2H), 3.28-3.26 (m, 4H), |
| | 3.01-2.94 (m, 2H), 2.79-2.65 (m, 3H), 0.89-0.70 |
| | (m, 3H), 0.45-0.11 (m, 12H). MS (ESI, m/e) |
| | [M + 1]⁺ 656.3 |
| 217 | ¹H NMR (400 MHz, DMSO-d₆) δ ppm: 10.23- |
| | 10.18 (m, 1H), 8.70-8.63 (m, 1H), 7.57-7.42 (m, |
| | 4H), 7.12-7.01 (s, 5H), 4.57-4.53 (m, 3H), 4.03 |
| | (s, 3H), 3.65-3.30 (m, 1H), 3.28-3.10 (m, 4H), |
| | 2.85-2.80 (m, 6H), 2.35-2.30 (m, 1H), 1.87-1.83 |
| | (m, 1H),1.55-1.23 (m, 1H). MS (ESI, m/e) |
| | [M + 1]⁺ 615.3 |
| 218 | ¹H NMR (400 MHz, DMSO-d₆) δ ppm: 10.09 |
| | (s, 1H), 9.45 (s, 1H), 8.42 (d, J = 8.0 Hz, 1H), |
| | 7.57 (s, 1H), 7.48 (s, 1H), 7.36-7.32 (m, 1H), |
| | 7.19-7.15 (m, 1H), 4.82-4.78 (m, 1H), 4.63-4.56 |
| | (m, 1H), 4.37-4.28 (m, 1H), 3.51-3.46 (m, 1H), |
| | 3.16-3.13 (m, 1H), 3.04-2.96 (m, 3H), 2.91-2.86 |
| | (m, 1H), 2.83-2.78 (m, 1H), 2.38 (s, 3H), 0.91- |
| | 0.76 (m, 2H), 0.67-0.63 (m, 1H), 0.47-0.42 (m, |
| | 1H), 0.39-0.33 (m, 1H), 0.31-0.26 (m, 2H), |
| | 0.22-0.17 (m, 2H), 0.12-0.07 (m, 1H). MS (ESI, |
| | m/e) [M + 1]⁺ 546.3 |
| 219 | ¹H NMR (400 MHz, DMSO-d₆) δ ppm: 10.22 |
| | (s, 1H), 9.22 (dd, J = 20.4 Hz, 5.2 Hz, 1H), 7.58- |
| | 7.53 (m, 2H), 7.47-7.30 (m, 1H), 7.24-7.13 (m, |
| | 1H), 6.88-6.83 (m, 1H), 4.58-4.53 (m, 1H), |
| | 3.75-3.62 (m, 2H), 3.28-3.20 (m, 2H), 3.10-3.00 |
| | (m, 2H), 2.90 (s, 2H), 2.69 (s, 1H), 2.48 (s, 3H), |
| | 2.19 (s, 3H), 2.13-1.64 (m, 7H), 1.52-1.30 (m, |
| | 2H), 0.53-0.37 (m, 4H). MS (ESI, m/e) [M + 1]⁺ |
| | 694.4. |
| 220 | ¹H NMR (400 MHz, DMSO-d₆) δ ppm: 10.21 |
| | (s, 1H), 9.32 (s, 1H), 9.22 (d, J = 8.6 Hz, 1H), |
| | 7.39-7.15 (m, 4H), 7.12-7.06 (m, 1H), 6.65 (s, |
| | 1H),4.57-4.53 (m, 1H), 3.65-3.30 (m, 2H), |
| | 3.28-3.10 (m, 4H),2.85-2.80 (m, 2H), 2.49-2.45 |
| | (m, 3H), 2.16 (s, 3H), 2.01-1.83 (m, 8H),1.55- |
| | 1.23 (m, 2H), 0.48-0.30 (m, 4H). MS (ESI, m/e) |
| | [M + 1]⁺ 696.4 |
| 221 | ¹H NMR (400 MHz, DMSO-d₆) δ ppm: 10.21 |
| | (s, 1H), 9.32 (s, 1H), 9.22 (d, J = 8.6 Hz, 1H), |
| | 7.39-7.15 (m, 4H), 7.12 (s, 3H), 6.65 (s, |
| | 1H),4.57-4.53 (m, 1H), 3.65-3.30 (m, 2H), |
| | 3.28-3.10 (m, 4H),2.85-2.80 (m, 2H), 2.75 (s, |
| | 3H), 2.49-2.45 (m, 3H), 2.16 (s, 3H), 2.01-1.83 |
| | (m, 6H), 1.55-1.23 (m, 2H), 0.48-0.30 (m, 4H). |
| | MS (ESI, m/e) [M + 1]⁺ 676.4 |
| 222 | ¹H NMR (400 MHz, DMSO-d₆) δ ppm: 10.03 |
| | (d, J = 4.6 Hz, 1H), 9.01 (dd, J = 8.8, 5.4 Hz, |
| | 1H), 8.40 (d, J = 6.8 Hz, 1H), 7.70 (s, 1H), 7.49- |
| | 7.36 (m, 2H), 7.33-7.21 (m, 1H), 7.13-7.08 (m, |
| | 2H), 6.77 (t, J = 6.6 Hz, 1H), 6.12 (s, 1H), 4.82 |
| | (t, J = 7.6 Hz, 1H), 3.92 (t, J = 14.8 Hz, 2H), |
| | 3.43-3.39 (m, 4H), 2.85 (s, 2H), 2.46 (s, 3H), |
| | 0.86-0.68 (m, 3H), 0.57-0.42 (m, 5H), 0.40-0.09 |
| | (m, 7H). MS (ESI, m/e) [M + 1]⁺ 635.2. |
| 223 | ¹H NMR (400 MHz, DMSO-d₆) δ ppm: 10.13 |
| | (s, 1H), 9.19 (d, J = 8.4 Hz, 1H), 8.40 (d, J = 6.8 |
| | Hz, 1H), 7.70 (s, 1H), 7.59-7.34 (m, 2H), 7.31- |
| | 7.24 (m, 1H), 7.13-7.08 (m, 2H), 6.78 (t, J = 6.8 |
| | Hz, 1H), 6.12 (s, 1H), 4.51 (t, J = 8.4 Hz, 1H), |
| | 3.94 (dd, J = 15.6, 9.8 Hz, 2H), 3.45-3.35 (m, |
| | 4H), 2.86 (s, 2H), 2.45 (s, 3H), 2.02-1.98 (m, |
| | 2H), 1.91-1.59 (m, 5H), 1.47-1.28 (m, 2H), 0.55 |
| | (s, 2H), 0.46 (s, 2H). MS (ESI, m/e) [M + 1]⁺ |
| | 659.5. |
| 224 | ¹H NMR (400 MHz, DMSO-d₆) δ ppm: 10.23- |
| | 10.18 (m, 1H), 8.70-8.63 (m, 1H), 7.57-7.42 (m, |
| | 4H), 7.12-7.01 (s, 4H), 6.75 (s, 1H), 4.57-4.53 |
| | (m, 3H), 4.03 (s, 3H), 3.65-3.30 (m, 1H), 3.28- |
| | 3.10 (m, 4H), 2.85-2.80 (m, 4H), 1.87-1.83 (m, |
| | 1H),1.55-1.23 (m, 1H), 0.89-0.70 (m, 4H). MS |
| | (ESI, m/e) [M + 1]⁺ 641.3 |
| 225 | ¹H NMR (400 MHz, DMSO-d₆) δ ppm: 10.08 |
| | (s, 1H), 9.01 (d, J = 8.0 Hz, 1H), 7.52-7.29 (m, |
| | 3H), 7.14 (d, J = 8.0 Hz, 1H), 4.90-4.82 (m, |
| | 1H), 4.31 (s, 1H), 3.85 (s, 1H), 3.58 (s, 1H), |
| | 3.43-3.33 (m, 4H), 3.22 (s, 3H), 3.11-3.04 (m, |
| | 2H), 2.53 (s, 3H), 0.85 (s, 2H), 0.78-0.72 (m, |
| | 1H), 0.46 (s, 1H), 0.38 (s, 1H), 0.31 (s, 1H), |
| | 0.25 (s, 2H), 0.16 (s, 1H). MS (ESI) m/e [M + 1]⁺ |
| | 591.4. |
| 226 | ¹H NMR (400 MHz, DMSO-d₆) δ ppm: 10.19 |
| | (s, 1H), 9.07 (d, J = 9.2 Hz, 1H), 7.58-7.53 (m, |
| | 1H), 7.47-7.38 (m, 1H), 7.24 (d, J = 8.4 Hz, |
| | 1H), 6.86 (s, 1H), 4.88-4.83 (m, 1H), 3.75-3.65 |
| | (m, 2H), 3.38-3.25 (m, 2H), 3.15 (s, 2H), 2.93 |
| | (s, 2H), 2.48 (s, 3H), 2.03-1.94 (m, 1H), 0.90- |
| | 0.69 (m, 4H), 0.51-0.11 (m, 10H). MS (ESI, |
| | m/e) [M + 1]⁺ 544.6. |
| 227 | ¹H NMR (400 MHz, DMSO-d₆) δ ppm: 10.24 |
| | (s, 1H), 9.38 -9.28 (m, 1H), 7.67-7.57 (m, 1H), |
| | 7.48 (s, 1H), 7.41-7.32 (m, 1H), 7.22-7.17 (m, |
| | 1H), 7.10 -7.03 (m, 1H), 6.73 (s, 1H), 4.72-4.56 |
| | (m, 2H), 4.38-4.28 (m, 1H), 3.55-3.45 (m, 1H), |
| | 3.20-3.13 (m, 2H), 3.08-2.70 (m, 7H), 2.48 (s, |
| | 3H), 2.05-1.80 (m, 2H), 1.48-1.24 (m, 2H), |
| | 0.99-0.65 (m, 4H). MS (ESI, m/e) [M + 1]⁺ |
| | 643.3. |
| 228 | ¹H NMR (400 MHz, DMSO-d₆) δ ppm: 10.21 |
| | (s, 1H), 9.24 (d, J = 8.6 Hz, 1H), 9.09 (d, J = 8.6 |
| | Hz, 1H), 8.24 (s, 1H), 7.57-7.42 (m, 4H), 7.12 |
| | (s, 2H), 6.65 (s, 1H),4.57-4.53 (m, 3H), 3.65- |
| | 3.30 (m, 2H), 3.28-3.10 (m, 5H),2.85-2.80 (m, |
| | 2H), 2.49-2.45 (m, 3H), 2.01-1.83 (m, |
| | 6H),1.55-1.23 (m, 2H), 0.48-0.30 (m, 2H), |
| | 0.17-0.12 (m, 2H). MS (ESI, m/e) [M + 1]⁺ 678.7 |
| 229 | ¹H NMR (400 MHz, DMSO-d₆) δ ppm: 10.21 |
| | (s, 1H), 9.32 (s, 1H), 9.22 (d, J = 8.6 Hz, 1H), |
| | 7.57-7.42 (m, 4H), 7.12 (s, 2H), 6.85 (s, 1H), |
| | 6.65 (s, 1H),4.57-4.53 (m, 1H), 3.65-3.30 (m, |
| | 2H), 3.28-3.10 (m, 4H),2.85-2.80 (m, 2H), 2.75 |
| | (s, 3H), 2.49-2.45 (m, 3H), 2.16 (s, 3H), 2.01- |
| | 1.83 (m, 5H), 1.55-1.23 (m, 2H), 0.48-0.30 (m, |
| | 4H). MS (ESI, m/e) [M + 1]⁺ 676.3 |
| 230 | MS (ESI, m/e) [M + 1]⁺ 587.4 |
| 231 | ¹H NMR (400 MHz, DMSO-d₆) δ ppm: 10.09 |
| | (s, 1H), 9.03 (d, J = 8.6 Hz, 1H), 7.51 (d, J = |
| | 20.8 Hz, 1H), 7.34 (dd, J = 18.4, 8.8 Hz, 1H), |
| | 7.16 (d, J = 8.2 Hz, 1H), 6.65 (s, 1H), 4.85 (t, J = |
| | 8.0 Hz, 1H), 4.60-4.54 (m, 1H), 3.24-3.22 (m, |
| | 2H), 2.97-2.80 (m, 6H), 2.49-2.47 (m, 3H), |
| | 1.52-1.48 (m, 1H), 0.83-075 (m, 8H), 0.46-0.20 |
| | (m, 8H). MS (ESI, m/e) [M + 1]⁺ 521.5. |
| 232 | ¹H NMR (400 MHz, DMSO-d₆) δ ppm: 10.18 |
| | (s, 1H), 9.22 (d, J = 8.2 Hz, 1H), 8.21 (s, 1H), |
| | 7.49-7.20 (m, 4H), 7.10-7.01 (m, 3H), 6.64 (s, |
| | 1H), 4.53 (t, J = 8.0 Hz, 1H), 3.47-3.40 (m, 2H), |
| | 3.08-2.96 (m, 4H), 2.87 (d, J = 10.6 Hz, 2H), |
| | 2.82-2.78 (m, 1H), 2.47 (s, 3H), 2.08-1.98 (m, |
| | 2H), 1.90-1.64 (m, 4H), 1.46-1.29 (m, 2H), 1.18 |
| | (s, 2H), 1.03-0.99 (m, 2H), 0.39 (s, 2H), 0.17- |
| | 0.13 (m, 2H). MS (ESI, m/e) [M + 1]⁺ 720.6. |
| 233 | ¹H NMR (400 MHz, DMSO-d₆) δ ppm: 10.18 (s, |
| | 1H), 9.22 (d, J = 8.4 Hz, 1H), 8.23 (s, 1H), 7.52- |
| | 7.25 (m, 3H), 7.15-7.01 (m, 3H), 6.65 (s, 1H), |
| | 4.54 (t, J = 8.2 Hz, 1H), 3.51-3.47 (m, 2H), |
| | 3.08-2.95 (m, 4H), 2.89 (d, J = 13.4 Hz, 2H), |
| | 2.82 (d, J = 10.2 Hz, 1H), 2.47 (s, 3H), 2.05- |
| | 2.02 (m, 2H), 1.76-1.65 (m, 4H), 1.43-1.35 (m, |
| | 2H), 1.29 (s, 2H), 1.04 (s, 2H), 0.39 (s, 2H), |
| | 0.15-0.12 (m, 2H). MS (ESI, m/e) [M + 1]⁺ |
| | 738.6. |
| 234 | ¹H NMR (400 MHz, DMSO-d₆) δ ppm: 10.11 |
| | (s, 1H), 9.48 (s, 1H), 9.04 (d, J = 8.8 Hz, 1H), |
| | 7.62-7.46 (m, 3H), 7.44-7.26 (m, 3H), 7.20-7.15 |
| | (m, 1H), 7.03 (t, J = 7.2 Hz, 1H), 6.93 (s, 1H), |
| | 4.88-4.83 (m, 1H), 3.79-3.67 (m, 1H), 3.60-3.53 |
| | (m, 1H), 3.45-3.34 (m, 2H), 3.32-3.25 (m, 1H), |
| | 3.18-3.11 (m, 1H), 2.87-2.80 (m, 1H), 2.03-1.94 |
| | (m, 1H), 1.53-1.44 (m, 1H), 0.90-0.69 (m, 8H), |
| | 0.51-0.11 (m, 9H). MS (ESI, m/e) [M + 1]⁺ |
| | 640.7. |
| 235 | ¹H NMR (400 MHz, DMSO-d₆) δ ppm: 10.10 |
| | (s, 1H), 9.48 (s, 1H), 8.43 (d, J = 8.8 Hz, 1H), |
| | 7.63-7.48 (m, 4H), 7.44-7.28 (m, 3H), 7.18-7.14 |
| | (m, 1H), 7.03 (t, J = 7.2 Hz, 1H), 6.93 (s, 1H), |
| | 5.44-5.38 (m, 1H), 4.88-4.83 (m, 1H), 3.79-3.67 |
| | (m, 1H), 3.60-3.53 (m, 1H), 3.48-3.38 (m, 2H), |
| | 3.32-3.25 (m, 2H), 3.15-3.07 (m, 1H), 2.87-2.83 |
| | (m, 1H), 1.53-1.34 (m, 7H), 0.93-0.69 (m, 9H), |
| | 0.50-0.05 (m, 8H). MS (ESI, m/e) [M + 1]⁺ |
| | 666.8. |
| 236 | ¹H NMR (400 MHz, DMSO-d₆) δ ppm: 10.11 |
| | (s, 1H), 9.68 (s, 1H), 9.04 (d, J = 8.8 Hz, 1H), |
| | 7.63-7.46 (m, 2H), 7.42-7.28 (m, 3H), 7.20-7.15 |
| | (m, 1H), 6.94 (s, 1H), 6.85 (t, J = 7.6 Hz, 1H), |
| | 4.88-4.83 (m, 1H), 3.79-3.67 (m, 1H), 3.60-3.53 |
| | (m, 1H), 3.45-3.34 (m, 2H), 3.32-3.25 (m, 2H), |
| | 3.15-3.07 (m, 1H), 2.87-2.80 (m, 1H), 2.48 (s, |
| | 3H), 1.53-1.44 (m, 1H), 0.90-0.69 (m, 9H), |
| | 0.51-0.11 (m, 8H). MS (ESI, m/e) [M + 1]⁺ |
| | 658.6. |
| 237 | ¹H NMR (400 MHz, DMSO-d₆) δ ppm: 10.12 |
| | (d, J = 8.8 Hz, 1H), 8.48 (d, J = 7.4 Hz, 1H), |
| | 7.51-7.45 (m, 2H), 7.20 (d, J = 22.6 Hz, 1H), |
| | 7.12-6.98 (m, 3H), 4.94-4.92 (m, 1H), 4.36 (t, J = |
| | 8.2 Hz, 2H), 4.02 (s, 3H), 3.79-3.64 (m, 1H), |
| | 3.58-3.33 (m, 2H), 3.30-3.14 (m, 3H), 2.86-2.79 |
| | (m, 1H), 1.83-1.80 (m, 2H), 1.69-1.67 (m, 2H), |
| | 1.59-1.56 (m, 2H), 1.15-1.14 (m, 4H). MS (ESI, |
| | m/e) [M + 1]⁺ 549.4. |
| 239 | ¹H NMR (400 MHz, DMSO-d₆) δ ppm: 10.09 |
| | (s, 1H), 8.85 (s, 1H), 8.38 (d, J = 12.0 Hz, 1H), |
| | 7.57 (s, 1H), 7.48 (s, 1H), 7.33-7.31 (m, 1H), |
| | 7.19-7.17 (m, 1H), 4.84-4.81 (m, 1H), 4.63-4.56 |
| | (m, 1H), 4.38-4.28 (m, 1H), 3.52-3.47 (m, 1H), |
| | 3.17-3.13 (m, 1H), 3.04-2.98 (m, 2H), 2.92-2.78 |
| | (m, 2H), 2.11 (s, 3H), 0.95-0.71 (m, 3H), 0.46- |
| | 0.13 (m, 8H). MS (ESI, m/e) [M + 1]⁺ 546.3 |
| 240 | ¹H NMR (400 MHz, DMSO-d₆) δ ppm: 10.26 (s, |
| | 1H), 9.10 (d, J = 8.4 Hz, 1H), 7.96 (s, 1H), 7.66 |
| | (s, 1H), 7.49 (d, J = 8.8 Hz, 1H), 7.39 (d, J = 8.8 |
| | Hz, 1H), 6.77 (s, 1H), 4.49 (t, J = 8.4 Hz, 1H), |
| | 3.65 (t, J = 9.8 Hz, 1H), 3.44-3.31 (m, 1H), 3.33 |
| | (s, 3H), 2.47 (s, 3H), 1.93-1.57 (m, 6H), 123- |
| | 1.04 (m, 5H). MS (ESI, m/e) [M + 1]⁺547.5 |
| 241 | MS (ESI, m/e) [M + 1]⁺629.6 |
| 242 | ¹H NMR (400 MHz, DMSO-d₆) δ ppm: 10.23 |
| | (s, 1H), 9.47 (s, 1H), 9.24 (d, J = 5.2 Hz, 1H), |
| | 7.63 -7.47 (m, 3H), 7.38-7.27 (m, 3H), 7.20- |
| | 7.15 (m, 1H), 7.03 (t, J = 7.2 Hz, 1H), 6.95 (s, |
| | 1H), 6.85 (t, J = 8.0 Hz, 1H), 4.58-4.53 (m, 1H), |
| | 3.72 (t, J = 15.6 Hz, 1H), 3.59-3.42 (m, 3H), |
| | 3.32-3.28 (m, 1H), 3.18-3.10 (m, 1H), 2.93-2.83 |
| | (m, 1H), 2.48 (s, 3H), 2.13-1.65 (m, 7H), 1.54- |
| | 1.30 (m, 3H), 0.85-0.78 (m, 3H), 0.75 (d, J = |
| | 6.4 Hz, 3H). MS (ESI, m/e) [M + 1]⁺ 664.7. |
| 243 | ¹H NMR (400 MHz, DMSO-d₆) δ ppm: 10.23 |
| | (s, 1H), 9.67 (s, 1H), 9.24 (d, J = 6.0 Hz, 1H), |
| | 7.63-7.47 (m, 2H), 7.45-7.27 (m, 3H), 7.17(t, |
| | J = 7.2 Hz, 1H), 6.96 (s, 1H), 6.85 (t, J = 8.0 Hz, |
| | 1H), 4.58-4.53 (m, 1H), 3.79-3.68 (m, 1H), |
| | 3.69-3.53 (m, 1H), 3.47-3.39 (m, 2H), 3.32-3.28 |
| | (m, 1H), 3.15-3.07 (m, 1H), 2.93-2.83 (m, 1H), |
| | 2.48 (s, 3H), 2.13-1.65 (m, 7H), 1.54-1.30 (m, |
| | 3H), 0.85-0.78 (m, 3H), 0.75 (d, J = 6.4 Hz, |
| | 3H). MS (ESI, m/e) [M + 1]⁺ 682.6. |
| 244 | ¹H NMR (400 MHz, DMSO-d₆) δ ppm: 10.07 |
| | (s, 1H), 9.01 (d, J = 8.8 Hz, 1H), 7.48 (s, 1H), |
| | 7.31 (d, J = 8.0 Hz, 1H), 7.12 (d, J = 8.0 Hz, |
| | 1H), 6.54 (s, 1H), 4.85 (t, J = 7.8 Hz, 1H), 3.53 |
| | (d, J = 10.6 Hz, 2H), 3.41 (d, J = 9.6 Hz, 1H), |
| | 3.27-3.16 (m, 6H), 3.04 (t, J = 14.8 Hz, 2H), |
| | 2.48 (s, 3H), 1.55-1.41 (m, 1H), 0.85 (s, 1H), |
| | 0.80-0.75 (m, 6H), 0.46 (s, 1H), 0.38 (s, 1H), |
| | 0.25-0.14 (m, 6H). MS (ESI) m/e [M + 1]⁺ 565.7. |
| 245 | ¹H NMR (400 MHz, DMSO-d₆) δ ppm: 10.06 |
| | (s, 1H), 8.40 (d, J = 8.8 Hz, 1H), 7.52-7.29 (m, |
| | 3H), 7.11 (d, J = 7.8 Hz, 1H), 7.05 (s, 1H), 6.53 |
| | (s, 1H), 4.79 (t, J = 8.0 Hz, 1H), 4.04 (s, 3H), |
| | 3.59-3.49 (m, 2H), 3.45-3.36 (m, 2H), 3.27-3.17 |
| | (m, 6H), 3.07-3.01 (m, 2H), 1.48 (d, J = 6.0 Hz, |
| | 1H), 0.84 (s, 1H), 0.80-0.70 (m, 6H), 0.44 (s, |
| | 1H), 0.35 (d, J = 6.2 Hz, 2H), 0.31-0.25 (m, |
| | 1H), 0.21 (s, 3H), 0.10 (s, 1H). MS (ESI) m/e |
| | [M + 1]⁺ 563.5. |
| 246 | ¹H NMR (400 MHz, DMSO-d₆) δ ppm: 10.04 |
| | (s, 1H), 8.98 (d, J = 8.4 Hz, 1H), 8.43 (s, 1H), |
| | 8.12-7.99 (m, 2H), 7.47 (d, J = 24.2 Hz, 1H), |
| | 7.32-7.27 (m, 1H), 7.15 (d, J = 5.8 Hz, 2H), 6.61 |
| | (s, 1H), 4.83 (s, 1H), 3.90-3.72 (m, 2H), 3.57- |
| | 3.54 (m, 3H), 3.30-3.15 (m, 2H), 2.47 (s, 3H), |
| | 1.48-1.39 (m, 1H), 0.84 (d, J = 6.6 Hz, 1H), |
| | 0.76-0.72 (m, 6H), 0.44 (s, 1H), 0.37 (s, 1H), |
| | 0.31 (s, 1H), 0.23-0.18 (m, 5H). MS (ESI) m/e |
| | [M + 1]⁺ 638.5. |
| 247 | ¹H NMR (400 MHz, DMSO-d₆) δ ppm: 10.17 |
| | (s, 1H), 9.22 (d, J = 8.4 Hz, 1H), 7.45 (d, J = 5.8 |
| | Hz, 1H), 7.32 (t, J = 7.2 Hz, 1H), 7.08 (d, J = |
| | 8.2 Hz, 1H), 6.83 (s, 1H), 6.61 (s, 1H), 4.54 (t, |
| | J = 8.4 Hz, 1H), 3.31-3.22 (m, 4H), 2.88 (s, 2H), |
| | 2.52 (d, J = 15.8 Hz, 2H), 2.47 (s, 3H), 2.07- |
| | 2.01 (m, 3H), 1.95-1.61 (m, 4H), 1.54-1.26 (m, |
| | 2H), 0.91 (s, 6H), 0.52 (s, 4H). MS (ESI, m/e) |
| | [M + 1]⁺ 601.3. |
| 248 | ¹H NMR (400 MHz, DMSO-d₆) δ ppm: 10.07 |
| | (s, 1H), 9.01 (d, J = 8.8 Hz, 1H), 7.57-7.44 (m, |
| | 2H), 7.38-7.32 (m, 1H), 7.14 (d, J = 7.6 Hz, |
| | 1H), 6.75 (s, 1H), 4.88-4.83 (m, 1H), 3.59-3.36 |
| | (m, 4H), 3.32-3.18 (m, 2H), 2.85-2.80 (m, 1H), |
| | 2.56-2.53 (m, 3H), 2.53 (s, 3H), 1.49-1.40 (m, |
| | 1H), 0.90-0.65 (m, 8H), 0.51-0.09 (m, 8H). MS |
| | (ESI, m/e) [M + 1]⁺ 578.8. |
| 249 | ¹H NMR (400 MHz, DMSO-d₆) δ ppm: 10.22 |
| | (s, 1H), 9.33-9.18 (m, 2H), 7.85 (s, 1H), 7.61- |
| | 7.31 (m, 2H), 7.28-7.06 (m, 5H), 4.55 (t, J = |
| | 8.4Hz, 1H), 3.70-3.36 (m, 3H), 3.31-3.19 (m, |
| | 1H), 3.04-2.94 (m, 1H), 2.69 (s, 1H), 2.47 (s, |
| | 3H), 2.10-1.96 (m, 3H), 1.88-1.70 (m, 2H), |
| | 1.55-1.42 (m, 3H), 1.36-1.16 (m, 2H), 0.82-0.73 |
| | (m, 6H). MS (ESI, m/e) [M + 1]⁺ 682.8. |
| 250 | Isomer A: ¹H NMR (400 MHz, DMSO-d₆) δ |
| | ppm: 10.13 (s, 1H), 9.05 (d, J = 8.8 Hz, 1H), |
| | 7.52 (s, 1H), 7.40 (d, J = 8.2 Hz, 1H), 7.18 (d, J = |
| | 8.2 Hz, 1H), 7.14 (s, 1H), 4.87 (t, J = 8.2 Hz, |
| | 1H), 3.61-3.55 (m, 2H), 3.43-3.17 (m, 5H), 2.91 |
| | (s, 1H), 2.49 (s, 3H), 1.35 (s, 3H), 1.15-1.08 (m, |
| | 1H), 0.95-0.74 (m, 2H), 0.73-0.67 (m, 1H), |
| | 0.51-0.33 (m, 8H), 0.33-0.04 (m, 6H). MS (ESI, |
| | m/e) [M + 1]⁺ 633.4. Retention time(Prep-HPLC): |
| | 8.0 min. |
| | Isomer B: ¹H NMR (400 MHz, DMSO-d₆) δ |
| | ppm: 10.14 (d, J = 5.2 Hz, 1H), 9.06 (d, J = 8.8 |
| | Hz, 1H), 7.64-7.33 (m, 2H), 7.28-7.20 (m, 1H), |
| | 7.17-7.18 (m, 1H), 4.87 (s, 1H), 3.63-3.56 (m, |
| | 2H), 3.39-3.21 (m, 5H), 3.09-2.96 (m, 1H), 2.50 |
| | (s, 3H), 1.24-1.11 (m, 4H), 0.99-0.65 (m, 6H), |
| | 0.55-0.10 (m, 11H). MS (ESI, m/e) [M + 1]⁺ |
| | 633.3. Retention time(Prep-HPLC): 8.8 min. |
| | Isomer C: ¹H NMR (400 MHz, DMSO-d₆) δ |
| | ppm: 10.13 (s, 1H), 9.04 (d, J = 8.8 Hz, 1H), |
| | 7.56 (s, 1H), 7.35 (d, J = 8.2 Hz, 1H), 7.18 (d, |
| | J = 8.2 Hz, 1H), 7.08 (s, 1H), 4.85 (t, J = 7.8 Hz, |
| | 1H), 3.59-3.52 (m, 1H), 3.41-3.36 (m, 2H), |
| | 3.23-3.00 (m, 5H), 2.49 (s, 3H), 1.41-1.36 (m, |
| | 4H), 0.97-0.66 (m, 8H), 0.51-0.12 (m, 9H). MS |
| | (ESI, m/e) [M + 1]⁺ 633.4. Retention time(Prep- |
| | HPLC): 9.4 min. |
| 251 | ¹H NMR (400 MHz, DMSO-d₆) δ ppm: 10.10 |
| | (s, 1H), 9.03 (d, J = 8.0 Hz, 1H), 7.57-7.29 (m, |
| | 4H), 7.19-7.15 (m, 1H), 7.11-7.07 (m, 1H), 6.61 |
| | (s, 1H), 4.87-4.83 (m, 1H), 3.85-3.64 (m, 2H), |
| | 3.36-3.37 (m, 3H), 3.23-3.16 (m, 1H), 2.97-2.91 |
| | (m, 1H), 2.48 (s, 3H), 0.89-0.67 (m, 10H), 0.49- |
| | 0.14 (m, 8H). MS (ESI, m/e) [M + 1]⁺ 615.6 |
| 252 | ¹H NMR (400 MHz, DMSO-d₆) δ ppm: 10.05 |
| | (s, 1H), 9.08-9.00 (m, 1H), 7.78 (s, 1H), 7.57- |
| | 7.48 (m, 2H), 7.43-7.30 (m, 2H), 7.23-6.93 (m, |
| | 1H), 4.88-4.83 (m, 1H), 3.79-3.36 (m, 6H), |
| | 3.32-3.18 (m, 1H), 2.48 (s, 3H), 2.05-1.88 (m, |
| | 2H), 1.52-1.45 (m, 2H), 0.96-0.70 (m, 10H), |
| | 0.51-0.06 (m, 8H). MS (ESI, m/e) [M + 1]⁺ |
| | 627.8. |
| 253 | ¹H NMR (400 MHz, DMSO-d₆) δ ppm: 10.23 |
| | (s, 1H), 9.25 (d, J = 8.6 Hz, 1H), 9.03 (d, J = 8.6 |
| | Hz, 1H), 7.54-7.42 (m, 3H), 7.20-7.01 (m, 5H), |
| | 4.57-4.53 (m, 4H), 3.65-3.30 (m, 1H), 3.28-3.26 |
| | (m, 1H), 2.75 (s, 3H), 2.49-2.45 (m, 3H), 2.16 |
| | (s, 3H), 2.01-1.83 (m, 5H), 1.55-1.23 (m, 5H), |
| | 0.79-0.50 (m,6H). MS (ESI, m/e) [M + 1]⁺ 678.5 |
| 254 | ¹H NMR (400 MHz, DMSO-d₆) δ ppm: 10.13 (s, |
| | 1H), 9.04 (d, J = 8.8Hz, 1H), 8.88 (s, 1H), 8.15 |
| | (d, J = 7.6 Hz, 1H), 7.56-7.33 (m, 2H), 7.24- |
| | 7.12 (m, 2H), 7.02 (d, J = 4.0 Hz, 2H), 6.89 (s, |
| | 1H), 4.88-4.84 (m, 1H), 3.79(s, 3H), 3.72-3.47 |
| | (m, 2H), 3.42-3.36 (m, 2H), 3.38-3.15 (m, 2H), |
| | 3.15-3.10 (m, 1H), 2.48 (s, 3H), 1.60-1.51 (m, |
| | 1H), 0.91-0.70 (m, 7H), 0.52-0.11 (m, 10H). |
| | MS (ESI, m/e) [M + 1]⁺ 670.8 |
| 255 | ¹H NMR (400 MHz, DMSO-d₆) δ ppm: 10.11 |
| | (d, J = 5.0 Hz, 1H), 9.05 (d, J = 8.8 Hz, 1H), |
| | 7.52 (d, J = 5.8 Hz, 1H), 7.18-7.08 (m, 2H), 6.98 |
| | (d, J = 8.0 Hz, 1H), 4.96-4.92 (m, 1H), 4.84 (t, |
| | J = 7.8 Hz, 1H), 4.36 (s, 1H), 3.79-3.66 (m, 1H), |
| | 3.59-3.38 (m, 2H), 3.30-3.14 (m, 2H), 2.87-2.82 |
| | (m, 1H), 2.48 (s, 3H), 0.90-0.66 (m, 3H), 0.47- |
| | 0.11 (m, 8H). MS (ESI, m/e) [M + 1]⁺ 563.5. |
| 257 | ¹H NMR (400 MHz, DMSO-d₆) δ ppm: 10.21 |
| | (d, J = 7.8 Hz, 1H), 9.23 (d, J = 8.5 Hz, 1H), |
| | 7.52 (d, J = 5.8 Hz, 1H), 7.22-7.08 (m, 2H), |
| | 7.01-6.96 (m, 1H), 4.95-4.94 (m, 1H), 4.52 (t, |
| | J = 8.2 Hz, 1H), 4.36 (s, 1H), 3.79-3.67 (m, 1H), |
| | 3.57-3.41 (m, 2H), 3.28-3.16 (m, 2H), 2.86-2.82 |
| | (m, 1H), 2.47 (s, 3H), 2.04-2.00 (m, 3H), 1.86- |
| | 1.65 (m, 4H), 1.47-1.30 (m, 2H). MS (ESI, m/e) |
| | [M + 1]⁺ 587.6. |
| 258 | ¹H NMR (400 MHz, DMSO-d₆) δ ppm: 10.18 |
| | (d, J = 7.5 Hz, 1H), 8.62 (d, J = 7.5 Hz, 1H), |
| | 7.54-7.44 (m, 2H), 7.23-6.99 (m, 4H), 4.95-4.93 |
| | (m, 1H), 4.48-4.37 (m, 2H), 4.03 (s, 3H), 3.79- |
| | 3.67 (m, 1H), 3.57-3.41 (m, 2H), 3.29-3.15 (m, |
| | 2H), 2.84 (d, J = 17.2 Hz, 1H), 2.05-2.01 (m, |
| | 3H), 1.88-1.63 (m, 4H), 1.43-1.29 (m, 2H). MS |
| | (ESI, m/e) [M + 1]⁺ 585.6. |
| 259 | ¹H NMR (400 MHz, DMSO-d₆) δ ppm: 10.11 |
| | (s, 1H), 9.65 (s, 1H), 9.08-9.00 (m, 1H), 7.78 (s, |
| | 1H), 7.57-7.48 (m, 2H), 7.43-7.30 (m, 2H), |
| | 7.23-7.09 (m, 2H), 6.93 (s, 1H), 4.88-4.83 (m, |
| | 1H), 3.79-3.36 (m, 4H), 3.32-3.18 (m, 2H), |
| | 2.85-2.80 (m, 1H), 2.48 (s, 3H), 1.52-1.45 (m, |
| | 1H), 0.96-0.70 (m, 9H), 0.51-0.09 (m, 8H). MS |
| | (ESI, m/e) [M + 1]⁺ 674.3. |
| 260 | ¹H NMR (400 MHz, DMSO-d₆) δ ppm: 10.13 |
| | (s, 1H), 10.01 (s, 1H), 9.08-9.04 (m, 1H), 8.06 |
| | (s, 1H), 7.77-7.75 (m, 2H), 7.53-7.30 (m, 2H), |
| | 7.25-7.20 (m, 1H), 6.99 (s, 1H), 4.88-4.83 (m, |
| | 1H), 3.79-3.36 (m, 4H), 3.32-3.18 (m, 1H), |
| | 2.85-2.80 (m, 1H), 2.48 (s, 3H), 0.96-0.70 (m, |
| | 10H), 0.51-0.09 (m, 8H). MS (ESI, m/e) |
| | [M + 1]⁺ 742.3. |
| 262 | ¹H NMR (400 MHz, DMSO-d₆) δ ppm: 10.13 |
| | (s, 1H), 9.29 (d, J = 8.6 Hz, 1H),9.02 (d, J = 8.6 |
| | Hz, 1H) 7.84-7.80 (m, 1H), 7.50-7.35 (m, 4H), |
| | 7.30-7.14 (m, 3H), 4.87-4.83 (m, 1H), 3.65-3.30 |
| | (m, 4H), 3.28-3.26 (m, 2H), 3.01-2.94 (m, 1H), |
| | 2.49-2.45 (m, 3H), 1.55-1.53 (m, 1H), 0.79-0.70 |
| | (m,9H), 0.45-0.11 (m, 8H). MS (ESI, m/e) |
| | [M + 1]⁺ 658.3 |
| 263 | ¹H NMR (400 MHz, DMSO-d₆) δ ppm: 10.13 |
| | (s, 1H), 9.17 (d, J = 8.6 Hz, 1H),9.06 (d, J = 8.6 |
| | Hz, 1H) 7.96-7.94 (m, 2H), 7.50-7.35 (m, 4H), |
| | 7.30-7.14 (m, 3H), 4.89-4.87 (m, 1H), 3.65-3.30 |
| | (m, 4H), 3.28-3.26 (m, 2H), 3.01-2.94 (m, 1H), |
| | 2.49-2.45 (m, 3H), 1.55-1.53 (m, 1H), 0.79-0.70 |
| | (m,9H), 0.29-0.11 (m, 7H). MS (ESI, m/e) |
| | [M + 1]⁺ 674.3 |
| 264 | ¹H NMR (400 MHz, DMSO-d₆) δ ppm: 10.12 |
| | (s, 1H), 9.23 (d, J = 6.0 Hz, 1H), 9.03 (d, J = 8.8 |
| | Hz, 1H), 7.96-7.88 (dd, J = 5.2, 8.8 Hz, 1H), |
| | 7.66 (d, J = 2.0 Hz, 1H), 7.56-7.46 (m, 1H), |
| | 7.45-7.33 (m, 2H), 7.23-7.11 (m, 2H), 4.85 (t, J = |
| | 7.6 Hz, 1H), 3.71-3.61 (m, 1H), 3.59-3.48 (m, |
| | 2H), 3.44-3.33 (m, 2H), 3.27-3.20 (m, 1H), |
| | 3.01-2.93 (m, 1H), 2.48 (s, 3H), 1.56-1.47 (m, |
| | 1H), 0.91-0.84 (m, 1H), 0.81-0.72 (m, 8H), |
| | 0.49-0.35 (m, 2H), 0.33-0.13 (m, 6H). MS (ESI, |
| | m/e) [M + 1]⁺ 708.6. |
| 265 | ¹H NMR (400 MHz, DMSO-d₆) δ ppm: 10.11 |
| | (s, 1H), 9.80 (s, 1H), 9.02 (dd, J = 3.6, 8.4 Hz, |
| | 1H), 7.75 (s, 2H), 7.56-7.46 (m, 1H), 7.44-7.31 |
| | (m, 1H), 7.25 (s, 1H), 7.19-7.15 (m, 1H), 6.96 |
| | (s, 1H), 4.92-4.80 (m, 1H), 3.77-3.72 (m, 1H), |
| | 3.61-3.51 (m, 1H), 3.47-3.33 (m, 2H), 3.28 (s, |
| | 1H), 3.07-3.02 (m, 1H), 2.85-2.78 (m, 1H), 2.49 |
| | (s, 3H), 1.48-1.45 (m, 1H), 0.88-0.84 (m, 1H), |
| | 0.82-0.69 (m, 8H), 0.51-0.36 (m, 2H), 0.33-0.12 |
| | (m, 6H). MS (ESI, m/e) [M + 1]⁺ 708.2. |
| 266 | ¹H NMR (400 MHz, DMSO-d₆) δ ppm: 10.07- |
| | 9.93 (m, 1H), 8.54-8.45 (m, 1H), 8.23-8.11 (m, |
| | 1H), 8.08-7.92 (m, 1H), 7.87-7.69 (m, 1H), |
| | 6.91-6.80 (m, 1H), 5.56-5.42 (m, 1H), 4.34-3.92 |
| | (m, 6H), 3.79-3.68 (m, 1H), 3.09-3.03 (m, 3H), |
| | 2.26-2.14 (m, 1H), 1.82-1.76 (m, 1H), 1.49-1.42 |
| | (m, 6H), 1.07-0.69 (m, 10H). MS (ESI, m/e) |
| | [M + 1]⁺ 546.5. |
| 267 | ¹H NMR (400 MHz, DMSO-d₆) δ ppm: 10.11 |
| | (s, 1H), 9.11-8.99 (m, 2H), 7.79-7.71 (m, 1H), |
| | 7.54-7.33 (m, 2H), 7.21-7.16 (m, 2H), 7.13-7.10 |
| | (m, 1H), 7.02 (d, J = 8.4 Hz, 1H), 4.85 (t, J = |
| | 8.0 Hz,1H), 3.76-3.44 (m, 4H), 3.42-3.40 (m, |
| | 1H), 3.27-3.21 (m, 1H), 3.02-2.91 (m, 1H), |
| | 2.50-2.48 (m, 3H), 1.96-1.86 (m, 1H), 1.57-1.46 |
| | (m, 1H), 0.94-0.92 (m, 2H), 0.89-0.84 (m, 1H), |
| | 0.82-0.71 (m, 8H), 0.67-0.64 (m, 2H), 0.49-0.36 |
| | (m, 2H), 0.33-0.13 (m, 6H). MS (ESI, m/e) |
| | [M + 1]⁺ 714.3. |
| 268 | ¹H NMR (400 MHz, DMSO-d₆) δ ppm: 10.12 |
| | (s, 1H), 9.58 (d, J = 8.4 Hz, 1H), 9.05 (d, J = 8.8 |
| | Hz, 1H), 7.59 (d, J = 7.6 Hz, 1H), 7.57-7.42 (m, |
| | 2H), 7.42-7.31 (m, 3H), 7.23-7.15 (m, 1H), |
| | 7.11-7.05 (m, 1H), 4.85 (t, J = 8.0 Hz, 1H), |
| | 3.62-3.52 (m, 1H), 3.79-3.51 (m, 1H), 3.44 (d, |
| | J = 6.0 Hz, 1H), 3.39 (s, 1H), 3.33-3.22 (m, 4H), |
| | 3.09 (d, J = 16.8 Hz, 1H), 2.89-2.80 (m, 1H), |
| | 2.48 (s, 3H), 1.51-1.42 (m, 1H), 0.91-0.79 (m, |
| | 1H), 0.78-0.67 (m, 8H), 0.50-0.28 (m, 2H), |
| | 0.28-0.10 (m, 4H). MS (ESI, m/e) [M + 1]⁺ |
| | 690.3. |
| 269 | ¹H NMR (400 MHz, DMSO-d₆) δ ppm: 10.11 |
| | (s, 1H), 9.62 (s, 1H), 9.08-8.99 (m, 1H), 7.64 (d, |
| | J = 1.8 Hz, 1H), 7.62 (d, J = 2.0 Hz, 1H), 7.55- |
| | 7.45 (m, 1H), 7.37-7.32 (m, 3H), 7.17 (dd, J = |
| | 4.8, 8.0 Hz, 1H), 6.93 (s, 1H), 4.89-4.81 (m, |
| | 1H), 3.79-3.67 (m, 1H), 3.60-3.51 (m, 1H), 3.43 |
| | (s, 1H), 3.39-3.37 (m, 1H), 3.31-3.27 (m, 1H), |
| | 3.10 (d, J = 16.8 Hz, 1H), 2.84 (q, J = 8.0 Hz, |
| | 1H), 2.48 (s, 3H), 2.07 (s, 1H), 1.51-1.41 (m, |
| | 1H), 0.91-0.82 (m, 1H), 0.78-0.75 (m, 4H), |
| | 0.72-0.69 (m, 4H), 0.50-0.42 (m, 1H), 0.41-0.35 |
| | (m, 1H), 0.34-0.17 (m, 6H), 0.17-0.11 (m, 1H). |
| | MS (ESI, m/e) [M + 1]⁺ 674.3. |
| 270 | ¹H NMR (400 MHz, DMSO-d₆) δ ppm: 10.10 |
| | (s, 1H), 9.02 (d, J = 8.8 Hz, 1H), 7.67-7.30 (m, |
| | 3H), 7.18 (dd, J = 18.6, 8.4 Hz, 1H), 7.09-6.92 |
| | (m, 1H), 4.93-4.84 (m, 2H), 4.27 (s, 1H), 3.71- |
| | 3.63 (m, 1H), 3.52-3.40 (m, 2H), 3.33-3.05 (m, |
| | 3H), 2.49 (s, 3H), 0.96-0.66 (m, 3H), 0.47-0.13 |
| | (m, 8H). MS (ESI, m/e) [M + 1]⁺ 645.4. |
| 271 | ¹H NMR (400 MHz, DMSO-d₆) δ ppm: 10.10 |
| | (s, 1H), 9.04 (d, J = 8.6 Hz, 1H), 7.19-7.04 (m, |
| | 2H), 6.99-6.9 (m, 1H), 6.68 (s, 1H), 4.89-4.87 |
| | (m, 2H), 3.50-3.02 (m, 8H), 2.89-2.80 (m, 1H), |
| | 2.49-2.45 (m, 2H), 1.55-1.33 (m, 1H), 0.79-0.70 |
| | (m,10H), 0.29-0.11 (m, 9H). MS (ESI, m/e) |
| | [M + 1]⁺ 537.5 |
| 272 | ¹H NMR (400 MHz, DMSO-d₆) δ ppm: 10.10 |
| | (s, 1H), 9.21-9.16 (m, 1H), 9.01 (d, J = 8.8 Hz, |
| | 1H), 7.80-7.73 (m, 1H), 7.73-7.64 (m, 2H), |
| | 7.56-7.47 (m, 1H), 7.42-7.33 (m, 2H), 7.20-7.11 |
| | (m, 2H), 4.89-4.83 (m, 1H), 3.72-3.61 (m, 1H), |
| | 3.57-3.48 (m, 2H), 3.46-3.39 (m, 1H), 3.35 (d, |
| | J = 6.8 Hz, 1H), 3.20 (dd, J = 2.8, 16.8 Hz, 1H), |
| | 2.97-2.88 (m, 1H), 2.48 (s, 3H), 1.54-1.47 (m, |
| | 1H), 0.91-0.84 (m, 1H), 0.83-0.69 (m, 8H), |
| | 0.50-0.13 (m, 8H). MS (ESI, m/e) [M + 1]⁺ |
| | 708.3. |
| 273 | ¹H NMR (400 MHz, DMSO-d₆) δ ppm: 10.08 |
| | (s, 1H), 9.00 (d, J = 8.8 Hz, 1H), 8.02-7.91 (dd, |
| | J = 3.2, 29.6 Hz, 1H), 7.47 (d, J = 8.0 Hz, 1H), |
| | 7.41-7.31 (m, 1H), 7.14 (d, J = 8.4 Hz, 1H), 6.85 |
| | (s, 1H), 4.89-4.82 (dd, J = 2.0, 7.2 Hz, 1H), |
| | 3.65-3.36 (m, 3H), 3.27-3.17 (m, 3H), 2.94-2.74 |
| | (m, 1H), 2.48 (s, 3H), 1.91-1.79 (m, 1H), 1.57- |
| | 1.38 (m, 2H), 0.87-0.66 (m, 10H), 0.49-0.36 (m, |
| | 2H), 0.31-0.12 (m, 6H). MS (ESI, m/e) [M + 1]⁺ |
| | 640.3. |
| 274 | ¹H NMR (400 MHz, DMSO-d₆) δ ppm: 10.09 |
| | (s, 1H), 9.46-9.36 (m, 1H), 9.01 (d, J = 8.8 Hz, |
| | 1H), 8.85 (d, J = 4.0 Hz, 1H), 8.32 (d, J = 5.6 |
| | Hz, 1H), 7.60 (d, J = 5.2 Hz, 1H), 7.56-7.47 (m, |
| | 1H), 7.44-7.33 (m, 1H), 7.19 (d, J = 7.2 Hz, |
| | 1H), 7.11 (s, 1H), 4.86 (dd, J = 7.2, 8.8 Hz, 1H), |
| | 3.76-3.64 (m, 1H), 3.61-3.48 (m, 2H), 3.47-3.40 |
| | (m, 1H), 3.21 (dd, J = 3.6, 16.8 Hz, 2H), 2.99- |
| | 2.91 (m, 1H), 2.48 (s, 3H), 1.55-1.46 (m, 1H), |
| | 0.92-0.84 (m, 1H), 0.81-0.72 (m, 8H), 0.50-0.43 |
| | (m, 1H), 0.41-0.38 (m, 1H), 0.34-0.30 (m, 1H), |
| | 0.29-0.12 (m, 5H). MS (ESI, m/e) [M + 1]⁺ |
| | 675.3. |
| 275 | ¹H NMR (400 MHz, DMSO-d₆) δ ppm: 10.10 |
| | (s, 1H), 9.40 (d, J = 8.4 Hz, 1H), 9.01 (d, J = 8.4 |
| | Hz, 1H), 7.55-7.46 (m, 1H), 7.40-7.31 (m, 2H), |
| | 7.32-7.31 (m, 1H), 7.28-7.22 (m, 1H), 7.20-7.15 |
| | (m, 1H), 6.97 (s, 1H), 4.86 (t, J = 7.6 Hz, 1H), |
| | 3.70-3.49 (m, 4H), 3.30-3.26 (m, 1H), 3.01-2.89 |
| | (m, 1H), 2.49-2.48 (m, 3H), 1.55-1.42 (m, 1H), |
| | 0.91-0.84 (m, 1H), 0.80-0.76 (m, 4H), 0.75-0.70 |
| | (m, 4H), 0.32 (br s, 3H), 0.30-0.13 (m, 6H). MS |
| | (ESI, m/e) [M + 1]⁺ 692.2 |
| 276 | ¹H NMR (400 MHz, DMSO-d₆) δ ppm: 10.12- |
| | 10.10 (m, 1H), 9.05 (m, 1H), 7.49-7.45 (m, 1H), |
| | 7.22 (s, 1H), 7.16-7.04 (m, 1H), 6.95 (d, J = 8.0 |
| | Hz, 1H), 4.95-4.84 (m, 2H), 4.42 (s, 1H), 3.99- |
| | 3.67 (m, 2H), 3.52-3.41 (m, 1H), 3.25-3.09 (m, |
| | 1H), 2.94-2.80 (m, 1H), 2.48 (s, 2H), 1.26-1.09 |
| | (m, 4H), 0.79-0.70 (m, 3H), 0.43-0.11(m, 8H). |
| | MS (ESI, m/e) [M + 1]⁺ 577.4. |
| 277 | ¹H NMR (400 MHz, DMSO-d₆) δ ppm: 10.12- |
| | 10.10 (m, 1H), 8.43 (d, J = 8.8 Hz, 1H), 7.52- |
| | 7.45 (m, 2H), 7.27-6.88 (m, 4H), 5.46-5.33 (m, |
| | 1H), 4.85-4.80 (m, 2H), 4.44-4.40 (m, 1H), |
| | 3.85-3.70 (m, 2H), 3.55-3.50 (m, 1H), 3.27-3.10 |
| | (m, 1H), 2.92-2.87 (m, 1H), 1.36 (dd, J = 13.8, |
| | 6.6 Hz, 6H), 1.25-1.19 (m, 1H), 1.15-1.09 (m, |
| | 2H), 0.88-0.68 (m, 3H), 0.49-0.03 (m, 8H). MS |
| | (ESI, m/e) [M + 1]⁺ 603.4. |
| 278 | ¹H NMR (400 MHz, DMSO-d₆) δ ppm: 10.11 |
| | (s, 1H), 9.22 (d, J = 6.4 Hz, 1H), 9.01 (d, J = 9.2 |
| | Hz, 1H), 7.95-7.86 (m, 1H), 7.58-7.47 (m, 2H), |
| | 7.44-7.34 (m, 1H), 7.22-7.16 (m, 2H), 7.05-6.98 |
| | (m, 1H), 4.85 (dd, J = 7.2, 8.4 Hz, 1H), 3.70- |
| | 3.55 (m, 2H), 3.54-3.49 (m, 1H), 3.42-3.36 (m, |
| | 1H), 3.29-3.23 (m, 1H), 3.01-2.98 (m, 1H), 2.48 |
| | (s, 3H), 1.54-1.51 (m, 1H), 0.91-0.85 (m, 1H), |
| | 0.82-0.72 (m, 8H), 0.50-0.10 (m, 9H). MS (ESI, |
| | m/e) [M + 1]⁺ 692.3. |
| 280 | ¹H NMR (400 MHz, DMSO-d₆) δ ppm: 10.07 |
| | (s, 1H), 9.00 (d, J = 8.8 Hz, 1H), 7.61 (s, 1H), |
| | 7.46 (d, J = 16.8 Hz, 1H), 7.35 (dd, J = 7.6, 18.4 |
| | Hz, 1H), 7.13 (d, J = 8.0 Hz, 1H), 6.85 (s, 1H), |
| | 4.93-4.78 (t, J = 7.6 Hz, 1H), 4.26-4.22 (m, 1H), |
| | 3.79-3.69 (m, 2H), 3.68-3.52 (m, 2H), 3.49-3.40 |
| | (m, 2H), 3.36 (s, 1H), 3.31-3.20 (m, 2H), 3.15- |
| | 3.04 (m, 1H), 2.86-2.80 (m, 1H), 2.48 (s, 3H), |
| | 2.10-1.97 (m, 1H), 1.80-1.70 (m, 1H), 1.48-1.37 |
| | (m, 1H), 0.91-0.83 (m, 1H), 0.81-0.65 (m, 8H), |
| | 0.50-0.35 (m, 2H), 0.34-0.12 (m, 6H). MS (ESI, |
| | m/e) [M + 1]⁺ 634.2. |
| 281 | ¹H NMR (400 MHz, DMSO-d₆) δ ppm: 8.81 (s, |
| | 1H), 8.47 (s, 1H), 7.87-7.81 (m, 1H), 7.69 (d, |
| | J = 8.0 Hz, 1H), 7.53 (d, J = 8.0 Hz, 1H), 7.27- |
| | 7.11 (m, 2H), 6.76-6.66 (m, 1H), 5.76-5.56 (m, |
| | 1H), 5.35-5.26 (m, 1H), 5.08-4.83 (m, 1H), |
| | 3.99-3.96 (m, 1H), 3.45-3.41 (m, 1H), 3.38-3.33 |
| | (m, 2H), 3.32-3.18 (m, 4H), 2.48 (s, 3H), 1.75- |
| | 1.52 (m, 3H), 1.01-0.29 (m, 22H). |
| 282 | ¹H NMR (400 MHz, DMSO-d₆) δ ppm: 10.07 |
| | (s, 1H), 9.01 (d, J = 8.8 Hz, 1H), 7.47 (d, J = |
| | 14.8 Hz, 1H), 7.37-7.30 (m, 2H), 7.14 (d, J = |
| | 8.0 Hz, 1H), 6.88 (s, 1H), 4.88-4.83 (m, 1H), |
| | 3.99-3.96 (m, 1H), 3.45-3.41 (m, 1H), 3.38-3.33 |
| | (m, 4H), 3.32-3.18 (m, 3H), 2.96-2.93 (m, 1H), |
| | 2.48 (s, 3H), 1.81-1.70 (m, 2H), 1.69-1.65 (m, |
| | 2H), 1.55-1.35 (m, 5H), 0.95-0.70 (m, 7H), |
| | 0.51-0.19 (m, 8H). MS (ESI, m/e) [M + 1]⁺ |
| | 632.6. |
| 283 | ¹H NMR (400 MHz, DMSO-d₆) δ ppm: 10.14- |
| | 10.09 (m, 1H), 9.70 (d, J = 3.6 Hz, 1H), 9.02 (d, |
| | J = 9.2 Hz, 1H), 7.83-7.79 (m, 1H), 7.67 (d, J = |
| | 2.0 Hz, 1H), 7.56-7.48 (m, 1H), 7.44-7.31 (m, |
| | 2H), 7.19 (d, J = 8.0 Hz, 1H), 7.07 (d, J = 2.0 |
| | Hz, 1H), 4.91-4.81 (m, 1H), 3.74 (t, J = 17.6 Hz, |
| | 1H), 3.61-3.54 (m, 1H), 3.53-3.39 (m, 2H), |
| | 3.31-3.27 (m, 1H), 3.16 (dd, J = 3.2, 16.8 Hz, |
| | 1H), 2.91-2.82 (m, 1H), 2.49 (br s, 3H), 1.55- |
| | 1.46 (m, 1H), 0.92-0.85 (m, 1H), 0.81-0.70 (m, |
| | 8H), 0.51-0.44 (m, 1H), 0.42-0.36 (m, 1H), |
| | 0.35-0.30 (m, 1H), 0.29-0.13 (m, 5H). MS (ESI, |
| | m/e) [M + 1]⁺ 665.3. |
| 284 | ¹H NMR (400 MHz, CDCl₃) δ ppm: 8.32-8.31 |
| | (m, 1H), 8.42-8.26 (m, 1H), 7.89-7.78 (m, 1H), |
| | 7.57-7.45 (m, 1H), 7.34 (s, 1H), 7.25-7.19 (m, |
| | 1H), 5.08-4.83 (m, 1H), 3.92-3.17 (m, 7H), |
| | 2.64-2.52 (m, 3H), 1.77-1.70 (m, 1H), 1.55-1.43 |
| | (m, 1H), 1.36-1.22 (m, 2H), 1.00-0.86 (m, 6H), |
| | 0.83-0.76 (m, 2H), 0.60-0.49 (m, 2H), 0.45-0.30 |
| | (m, 2H), 0.28-0.14 (m, 2H). MS (ESI, m/e) |
| | [M + 1]⁺ 675.3. |
| 285 | ¹H NMR (400 MHz, DMSO-d₆) δ ppm: 10.10 |
| | (s, 1H), 9.11-8.88 (m, 1H), 8.53 (s, 1H), 7.67 (s, |
| | 1H), 7.57-7.45 (m, 1H), 7.45-7.24 (m, 2H), |
| | 7.18-7.14 (m, 1H), 6.95-6.91 (m, 1H), 3.48-3.39 |
| | (m, 1H), 3.30-3.23 (m, 2H), 3.17-3.05 (m, 1H), |
| | 2.85-2.80 (m, 1H), 2.45-2.40 (m, 3H), 1.50-1.42 |
| | (m, 1H), 0.90-0.83 (m, 1H), 0.83-0.73 (m, 5H), |
| | 0.71-0.69 (m, 3H), 0.50-0.43 (m, 1H), 0.41-0.35 |
| | (m, 1H), 0.34-0.11 (m, 6H). MS (ESI, m/e) |
| | [M + 1]⁺ 647.3. |
| 286 | ¹H NMR (400 MHz, DMSO-d₆) δ ppm: 9.83 (s, |
| | 1H), 8.37 (d, J = 9.2 Hz, 1H), 7.49 (s, 1H), 6.95- |
| | 6.81 (m, 3H), 6.69 (d, J = 7.8 Hz, 1H), 6.62 (s, |
| | 1H), 5.73 (s, 1H), 5.40 (s, 1H), 4.76 (t, J = 9.4 |
| | Hz, 1H), 3.25-2.87 (m, 8H), 1.55-1.43 (m, 1H), |
| | 1.51-1.42 (m, 6H), 0.91-0.72 (m, 10H), 0.44- |
| | 0.10 (m, 8H). MS (ESI, m/e) [M + 1]⁺ 562.5. |
| 287 | ¹H NMR (400 MHz, DMSO-d₆) δ ppm: 9.89 (d, |
| | J = 7.2 Hz, 1H), 8.33 (d, J = 8.8 Hz, 1 H), 7.50 |
| | (s, 1 H), 6.95-6.74 (m, 4 H), 6.64 (d, J = 9.4 Hz, |
| | 1 H), 5.41 (s, 1 H), 4.77 (t, J = 8.4 Hz, 1 H), |
| | 3.55-3.37 (m, 3 H), 3.28-2.90 (m, 4 H), 2.69 (d, |
| | J = 4.4 Hz, 3 H),2.66-2.62 (m, 1 H), 1.36 (dd, J = |
| | 11.6, 6.6 Hz, 6H), 0.89-0.65 (m, 10 H), 0.35- |
| | 0.11 (m, 8H). MS (ESI, m/e) [M + 1]⁺ 576.5. |
| 288 | ¹H NMR (400 MHz, CDCl₃) δ ppm: 8.70-8.57 |
| | (m, 1H), 8.41-8.28 (m, 1H), 7.81-7.75 (m, 1H), |
| | 7.72-7.68 (m, 1H), 7.52-7.45 (m, 1H), 7.39-7.27 |
| | (m, 1H), 7.23-7.18 (m, 2H), 7.04 (br s, 2H), |
| | 7.05-7.03 (m, 1H), 5.63-5.21 (m, 1H), 4.96-4.86 |
| | (m, 1H), 4.11-3.93 (m, 1H), 3.90-3.79 (m, 1H), |
| | 3.59-3.26 (m, 4H), 2.88-2.78 (m, 1H), 2.77-2.67 |
| | (m, 1H), 2.60-2.58 (m, 3H), 1.71-1.65 (m, 1H), |
| | 1.13-1.05 (m, 6H), 0.99-0.75 (m, 12H), 0.62- |
| | 0.50 (m, 2H), 0.35-0.27 (m, 2H). MS (ESI, m/e) |
| | [M + 1]⁺ 682.5. |
| 289 | ¹H NMR (400 MHz, DMSO-d₆) δ ppm: 10.08 |
| | (s, 1H), 9.05-8.99 (m, 2H), 7.57-7.47 (m, 1H), |
| | 7.46-7.34 (m, 2H), 7.23-7.17 (m, 2H), 7.16-7.08 |
| | (m, 2H), 7.06-7.02 (m, 1H), 4.86 (dd, J = 7.2, |
| | 8.4 Hz, 1H), 3.70-3.67 (m, 1H), 3.62-3.50 (m, |
| | 2H), 3.46-3.39 (m, 1H), 3.03-2.90 (m, 1H), |
| | 1.54-1.44 (m, 1H), 1.09 (t, J = 7.6 Hz, 3H), |
| | 0.92-0.83 (m, 1H), 0.79-0.72 (m, 8H), 0.51-0.12 |
| | (m, 8H). MS (ESI, m/e) [M + 1]⁺ 668.7. |
| 290 | Isomer A: ¹H NMR (400 MHz, DMSO-d₆) δ |
| | ppm: 10.07 (s, 1H), 8.42 (d, J = 8.8 Hz, 1H), |
| 291 | 7.53-7.50 (m, 2H), 7.22-6.89 (m, 4H), 5.43-5.40 |
| | (m, 1H), 4.99-4.97 (m, 1H), 4.77 (s, 1H), 4.39- |
| | 4.37 (m, 1H), 3.87-3.85 (m, 1H), 3.08-3.04(m, |
| | 3H), 1.38-1.33 (m, 6H), 1.12-0.99 (m, 1H), |
| | 0.79-0.70 (m, 4H), 0.52-0.04 (m, 11H). MS |
| | (ESI, m/e) [M + 1]⁺ 629.4. Retention time(Prep- |
| | HPLC): 7.4 min |
| | Isomer B: ¹H NMR (400 MHz, DMSO-d₆) δ |
| | ppm: 10.11 (s, 1H), 8.44 (d, J = 8.8 Hz, 1H), |
| | 7.51-7.40 (m, 2H), 7.27 (s, 1H), 7.17-7.08 (m, |
| | 1H), 7.02-6.88 (m, 2H), 5.48-5.31 (m, 1H), |
| | 4.98-4.95 (m, 1H), 4.78-4.76 (m, 1H), 4.40-4.35 |
| | (m, 1H), 3.85 (s, 1H), 3.63-3.60 (m, 1H), 3.24- |
| | 2.99 (m, 3H), 1.40-1.30 (m, 6H), 1.05 (s, 1H), |
| | 0.91-0.69 (m, 3H), 0.54-0.50 (m, 3H), 0.40-0.15 |
| | (m, 7H). MS (ESI, m/e) [M + 1]⁺ 629.4. |
| | Retention time(Prep-HPLC): 8.2 min |
| | ¹H NMR (400 MHz, DMSO-d₆) δ ppm: 10.15 |
| | (s, 1H), 8.54 (d, J = 8.4 Hz, 1H), 7.58-7.50 (m, |
| | 2H), 7.31-6.98 (m, 4H), 4.47-4.43 (m, 2H), |
| | 4.20-4.05 (m, 4H), 3.80-3.77 (m, 2H), 3.53-3.50 |
| | (m, 1H), 3.24-3.22 (m, 1H), 3.09-3.08 (m, 1H), |
| | 2.76-2.73 (m, 1H), 2.49-2.28 (m, 2H), 1.95-1.65 |
| | (m, 6H), 1.82-1.58 (m, 4H), 1.24-1.20 (m, 3H). |
| | MS (ESI, m/e) [M + 1]⁺ 563.4. |
| 292 | Isomer A: ¹H NMR (400 MHz, DMSO-d₆) δ |
| | ppm: 10.10 (s, 1H), 9.03 (d, J = 8.6 Hz, 1H), |
| | 7.61-7.28 (m, 2H), 7.17 (d, J = 7.6 Hz, 1H), 6.59 |
| | (s, 1H), 4.85 (t, J = 8.0 Hz, 1H), 4.01 (s, 1H), |
| | 3.51-3.36 (m, 2H), 3.27-3.11 (m, 3H), 3.07-3.00 |
| | (m, 2H), 2.81-2.77 (m, 1H), 2.48 (s, 3H), 1.35- |
| | 1.23 (m, 3H), 0.93-0.58 (m, 10H), 0.48-0.14 (m, |
| | 8H). MS (ESI, m/e) [M + 1]⁺ 632.4. Retention |
| | time(Prep-HPLC): 7.9 min. |
| | Isomer B: ¹H NMR (400 MHz, DMSO-d₆) δ |
| | ppm: 10.10 (s, 1H), 9.04 (d, J = 8.8 Hz, 1H), |
| | 7.53 (t, J = 14.6 Hz, 1H), 7.43-7.27 (m, 1H), |
| | 7.20-7.12 (m, 1H), 6.55 (s, 1H), 4.85 (t, J = 7.8 |
| | Hz, 1H), 4.14-4.08 (m, 1H), 3.52-3.45 (m, 3H), |
| | 3.26-3.17 (m, 2H), 3.19-3.03 (m, 2H), 2.84-2.76 |
| | (m, 1H), 2.59 (s, 1H), 2.48 (s, 3H), 1.26-1.18 |
| | (m, 3H), 0.92-0.66 (m, 4H), 0.50-0.45 (m, 6H), |
| | 0.37-0.11 (m, 7H). MS (ESI, m/e) [M + 1]⁺ |
| | 632.4. Retention time(Prep-HPLC): 9.4 min. |
| 293 | ¹H NMR (400 MHz, DMSO-d₆) δ ppm: 10.00 |
| | (d, J = 8.8 Hz, 1H), 9.01-8.96 (m, 1H), 7.52 (s, |
| | 1H), 7.48 (s, 1H), 7.24 (t, J = 8.4 Hz, 1H), 6.70 |
| | (t, J = 8.8 Hz, 1H), 4.98-4.80 (m, 2H), 4.42-4.30 |
| | (m, 1H), 3.79-3.65 (m, 1H), 3.59-3.37 (m, 2H), |
| | 3.32-3.15 (m, 2H), 2.95-2.85 (m, 1H), 2.42 (s, |
| | 3H), 0.90-0.68 (m, 3H), 0.49-0.12 (m, 8H). MS |
| | (ESI, m/e) [M + 1]⁺ 563.4. |

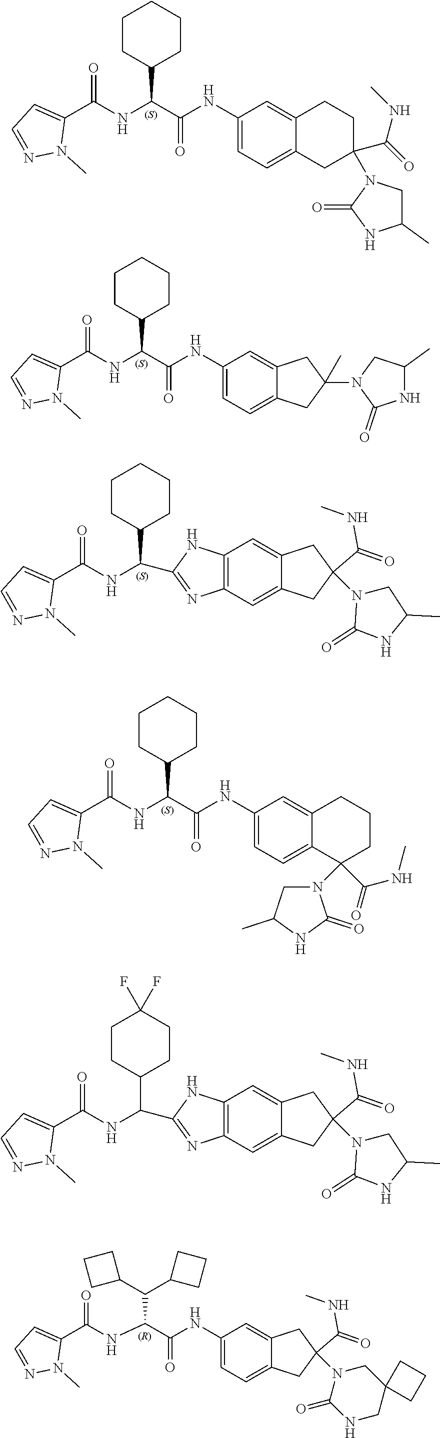

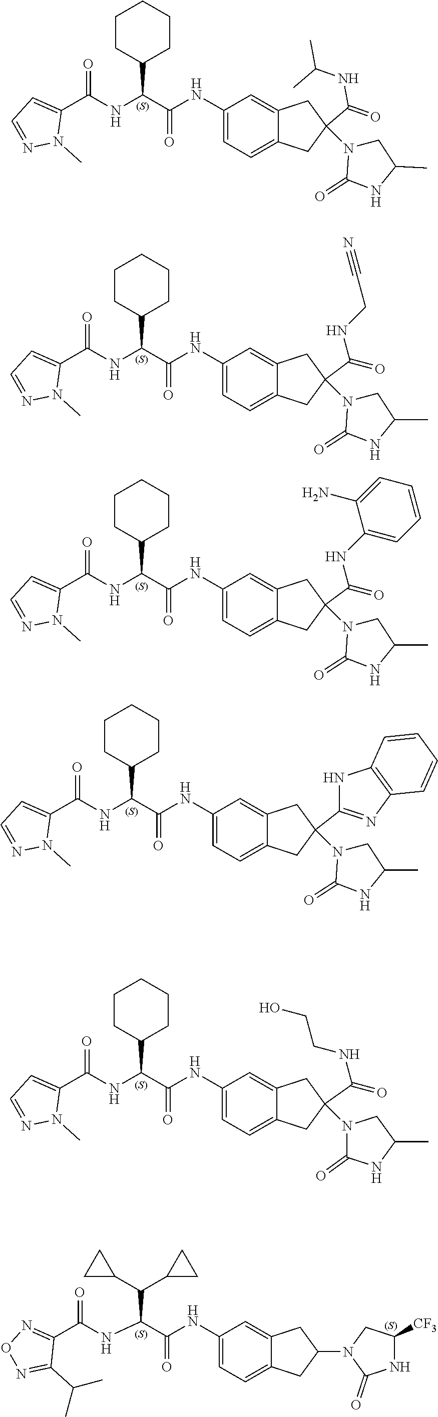

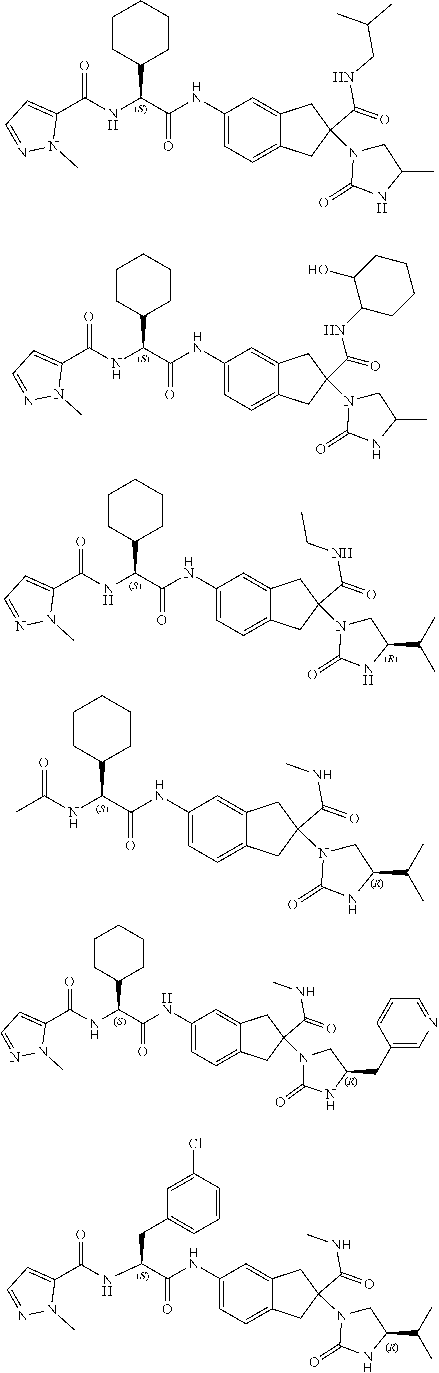

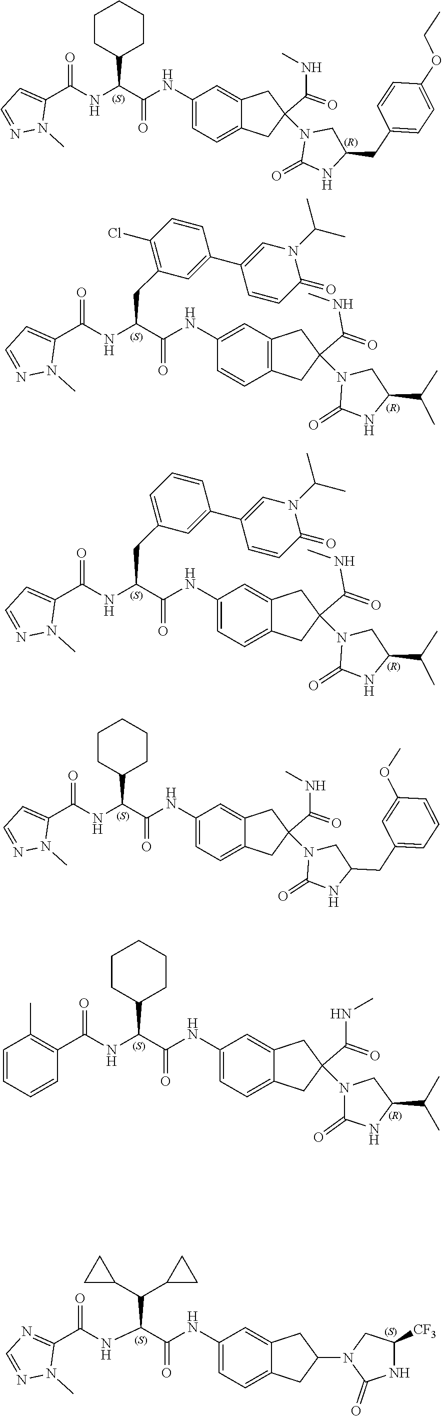

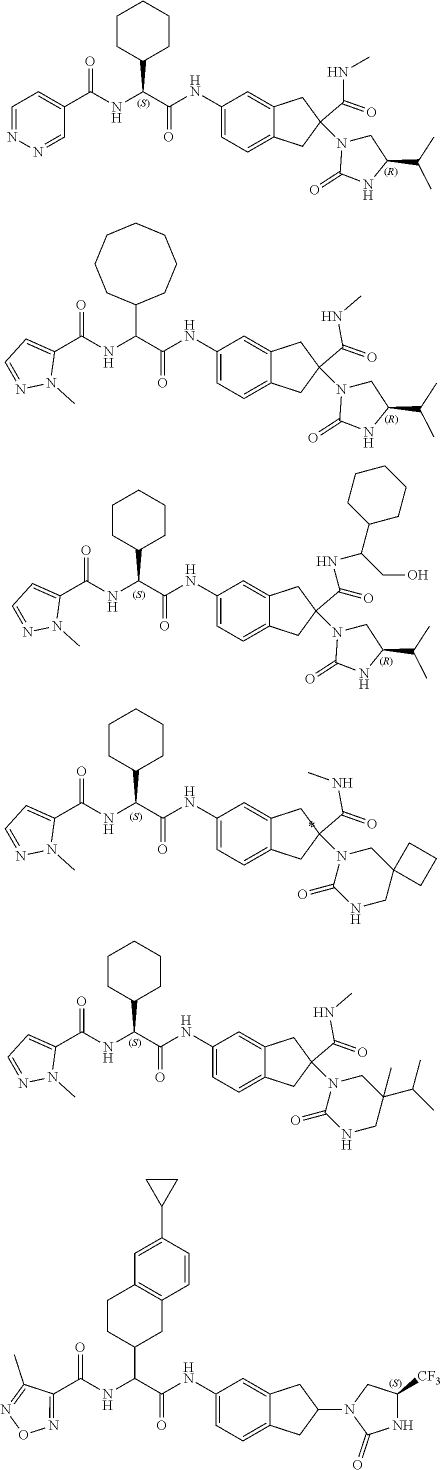

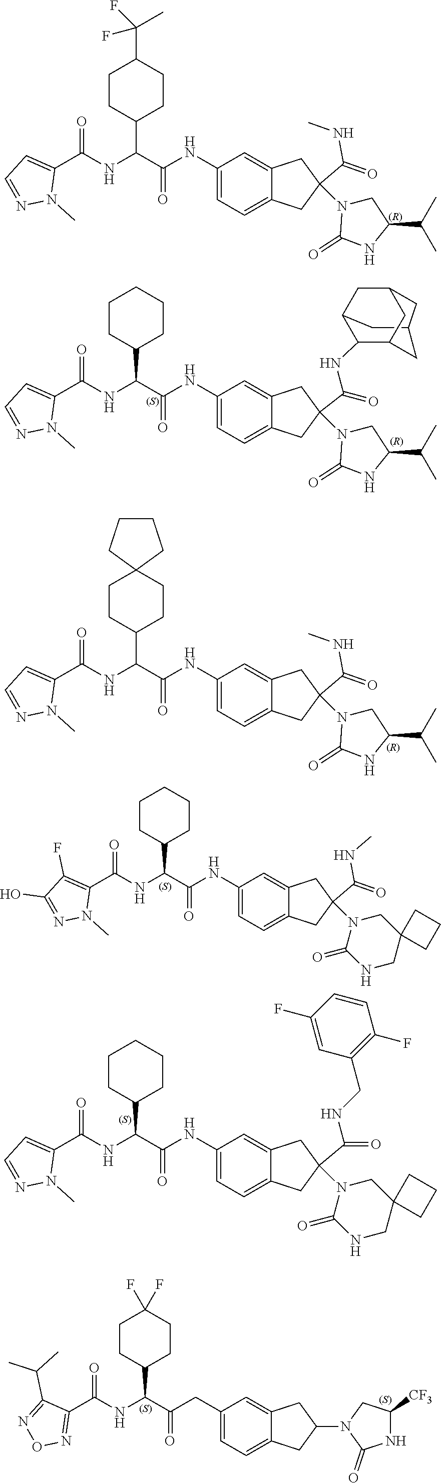

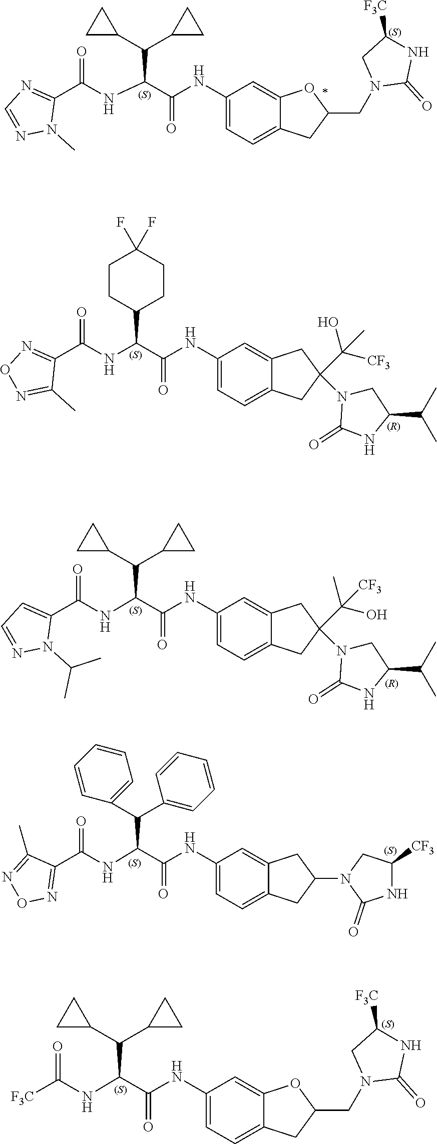

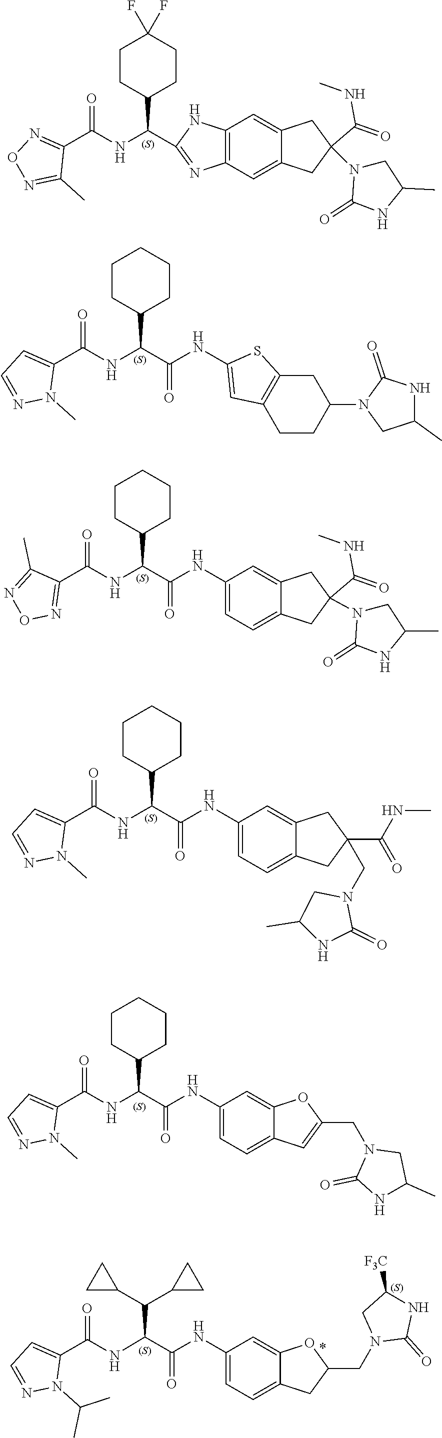

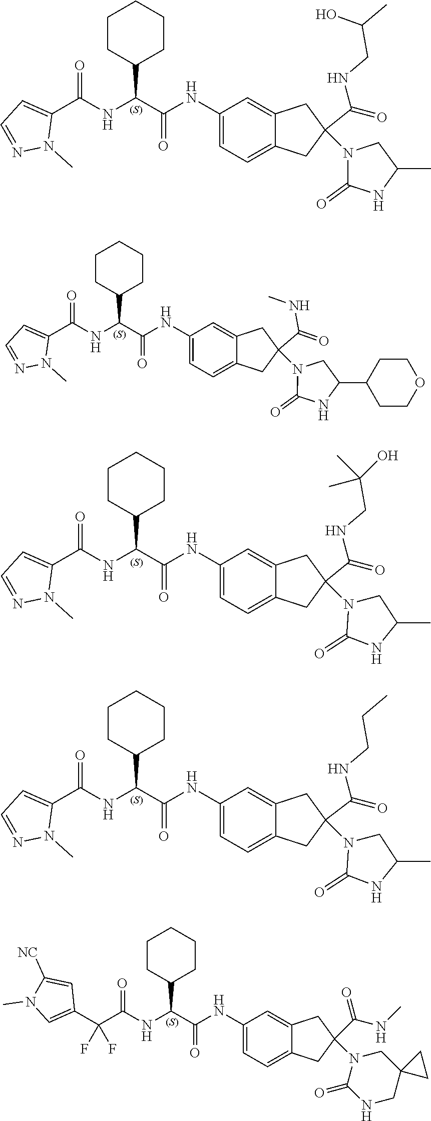

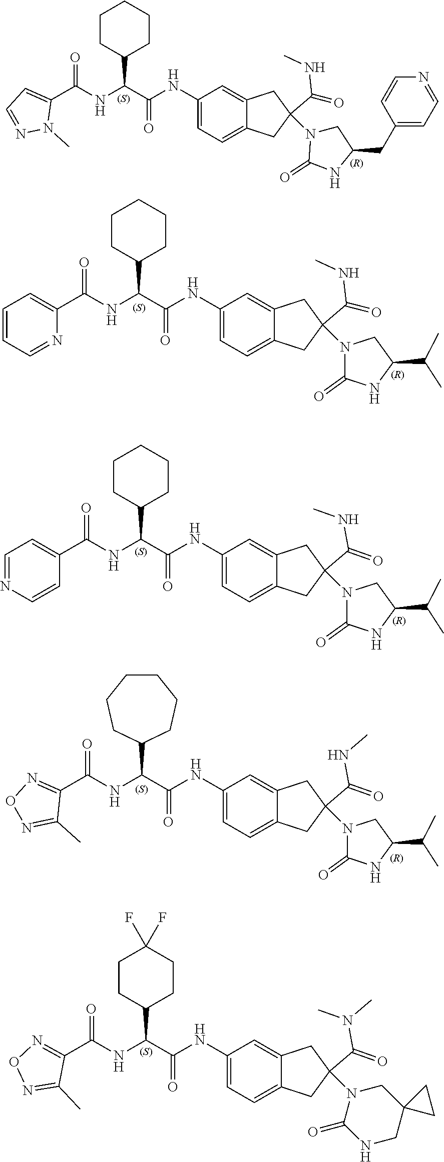

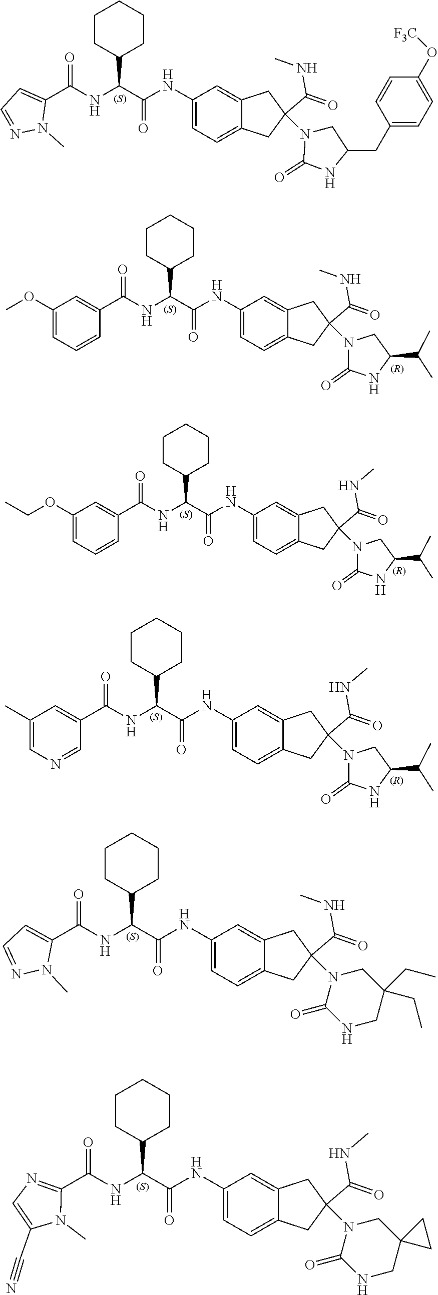

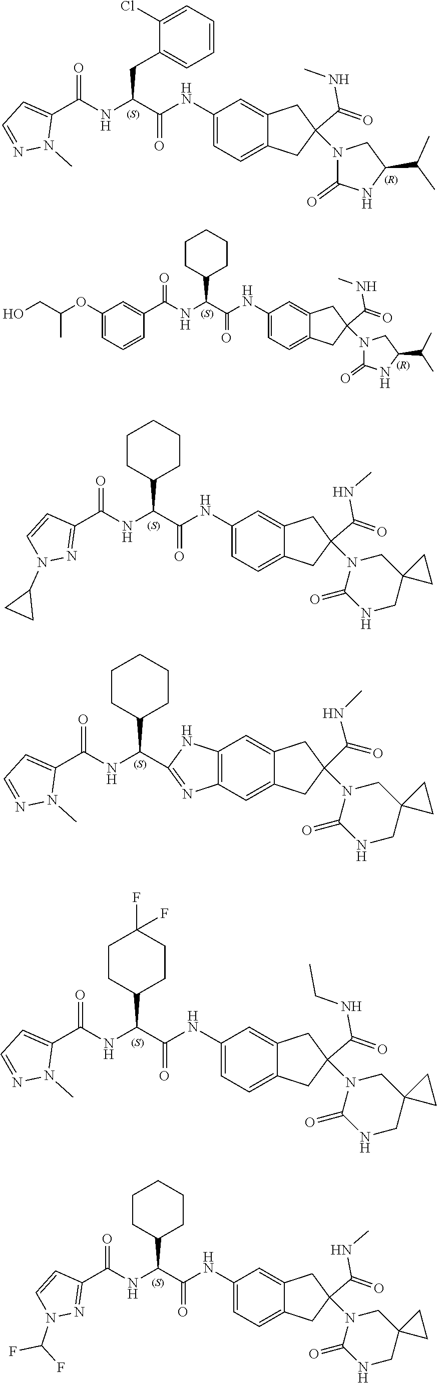

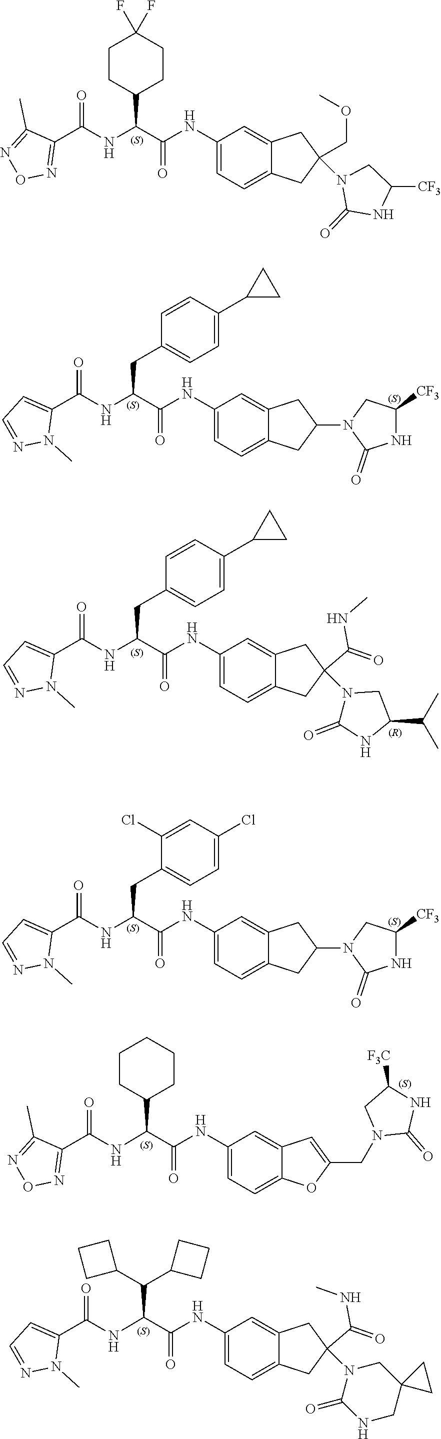

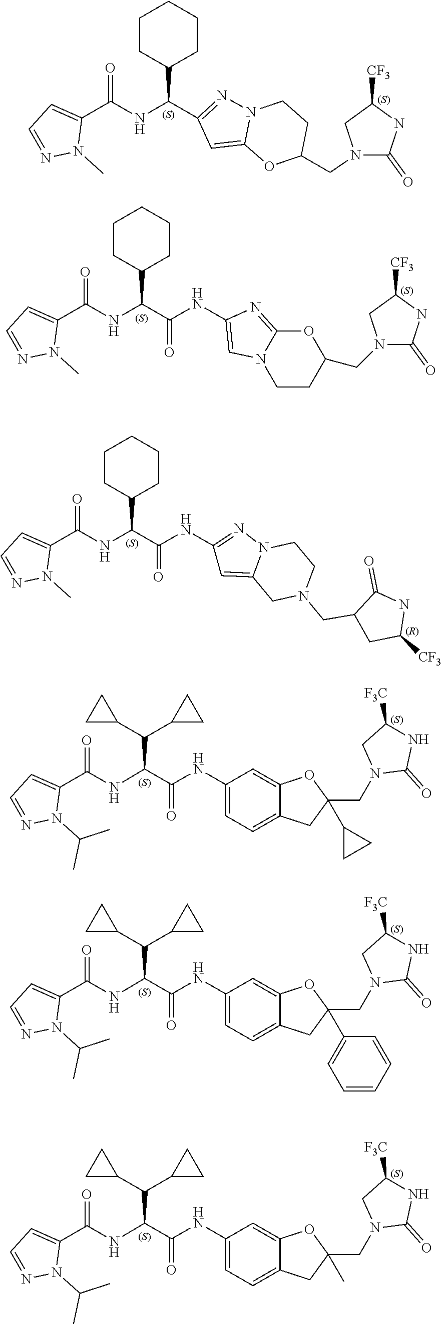

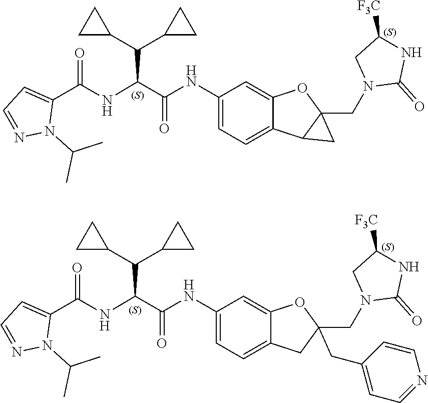

### Biological Assays:

The biological activity of the compounds of the present disclosure was determined utilizing the assays described herein. Values may fluctuate depending on the daily assay performance, fluctuations of this kind a known to those skilled in the art. These results show that the compounds of the present disclosure are capable of inhibiting the biological action of IL-17A.

### Human IL-17A Biochemical Assay

Compounds disclosed herein were tested for blocking of human IL-17A (Cat: C774, novoprotein) protein with its receptor human IL-17RA (Cat: C153, novoprotein) in an assay based on Homogeneous Time Resolved Fluorescence. 0.4 nM recombinant human IL-17A protein was pre-incubated with a serial dilution of compounds disclosed herein (maximum concentration is 10 uM, 2.7-fold serially diluted, 10 points) at room temperature for 3 hours in an assay buffer containing 20 mM HEPES, pH 7.5, 50 mM NaCl, 0.1% BSA, 0.2 mM DTT, 0.005% Tween 20. Then 0.3 nM recombinant human IL-17RA was added to plate and further incubated at room temperature for 1 hour. After that Mab Anti-6His Tb cryptate Gold (Cat: 61HI2TLB, Cisbio Bioassays) and MAb Anti Human IgG-XL665 (Cat: 61HFCXLB, Cisbio Bioassays) were added to plate and further incubated at room temperature for 1 hour. The HTRF signals (ex337 nm, em620 nm/665 nm) were read on BMG PHERAstar FSX instrument. The inhibition percentage of human IL-17A interaction with its receptor human IL-17RA in presence of increasing concentrations of compounds was calculated based on the ratio of fluorescence at 620 nm to that at 665 nm. The IC50 for each compound was derived from fitting the data to the four-parameter logistic equation by Dotmatics.

### GROα Release Assay in Human Colorectal Adenocarcinoma Epithelial Cell

The purpose of the assay is to test the ability of a compound to neutralize IL17A proteins. IL17 can stimulate human epithelial cells to secrete GROα. The ability of one compound of the invention to neutralize IL-17-induced GROα secretion from the human colorectal adenocarcinoma epithelia cell line HT-29 is tested in this assay.

HT-29 cells (human colorectal adenocarcinoma epithelia cell, ATCC) were stimulated with IL-17A (Novoprotein, #C774) in final concentration of 10 ng/mL. The resultant GROα response was measured using an ELISA kit from Invitrogen (#88-52122). HT-29 cells were cultured in complete medium (McCoy's 5A medium+10% FBS) and maintained in a tissue culture flask using standard techniques. On day 1, detached the cell by using TrypLE (Gibco, #12605036) and used the complete medium to neutralize. The cells were centrifuged at 11,00 rmp for 4 minutes. Re-suspended the cell pellet in complete medium and seeded 50,000 HT-29 cells into the 96-well assay plate (Corning, 3599) at 135 μL complete medium per well. The cells were incubated for overnight, at 37° C./5% CO₂ to adherent the plate. On day 2, prepared a 4-fold serial dilution of the tested compound in DMSO. The highest concentration of the compound was 10 μM. Transferred 2 μL of the serial diluted compound from the serial dilution plate into the reagent plate which was contained 198 μL complete medium with 100 ng/mL IL-17A per well. The compounds were incubated with the IL-17A for 15 minutes. 15 μL mixtures from reagent plate were transferred into the cell assay plate. Incubated the plate at 37° C./5% CO₂ for 48 hours. After the incubation, on day 4, GROα level were measured with GROα ELISA as the manufacturer's instructions. 50 μL supernatant was collected form the assay plate to ELISA plate. ELISA plate was read at 450 nm on a microplate reader and compared to a standard calibration curve.

Data were processed using GraphPad Prism. GROα concentration was calculated from the standard curve. IC50 was determined using the equation log concentration (inhibitor) against response and fitted to four parameters.

| | Biochemical | | | | |
|---|---|---|---|---|---|
| | assay | Cellular assay | | | |
| | IL-17A/IL- | HT29 cell/ | | Biochemical assay | Cellular assay |
| | 17RA | CXCL1 | | IL-17A/IL-17RA | HT29 cell/CXCL1 |
| EX | IC50 (nM) | IC50 (nM) | EX | IC50 (nM) | IC50 (nM) |
| 1 | 378.7 | 897.5 | 149 | 92 | 44.3 |
| 2 | 1,100 | 2,565 | 150 | 20 | 46.5 |
| 3 | 3,190 | 8,434 | 151 | 31 | 99.6 |
| 4 | >10,000 | | 152 | 10 | 31.0 |
| 5 | 410 | 352.7 | 153 | 95 | 258.2 |
| 6 | 17,000 | | 154 | 28 | 71.4 |
| 7 | 51,000 | | 155 | 129 | 210.9 |
| 8 | >10,000 | | 156 | 68.3 | 305.3 |
| 9 | 8,000 | >10,000 | 157 | 37 | 398.3 |
| 10 | 19,000 | | 158 | 123.1 | 48.5 |
| 11 | 1,700 | 6,522 | 159 | 53.2 | 186.7 |
| 12 | 13,000 | >10,000 | 160 | 110.5 | 93.1 |
| 13 | 1,800 | 1,495 | 161 | 120.7 | 145.5 |
| 14 | 150 | 300.3 | 162 | 539.9 | 1,063 |
| 15 | 550 | 1,235 | 163 | 19.0 | 38.8 |
| 16 | 5,000 | | 164 | 35.2 | 111.8 |
| 17 | 2,500 | 2,636 | 165 | 173.4 | 322.2 |
| 18 | 1,000 | 2,553 | 166 | 168.8 | 135 |
| 19 | 60 | 262.3 | 167 | 127 | 219.6 |
| 20 | 980 | 475.6 | 168 | 32.8 | 60.5 |
| 21 | 2,400 | | 169 | 56.4 | 65.2 |
| 22 | 950 | 7,117 | 170 | 168.6 | 226.7 |
| 23 | 640 | 3,662 | 171 | 7.0 | 9.7 |
| 24 | 2,342 | | 172 | 400.3 | 376.9 |
| 25 | 6,300 | | 173 | | 153.1 |
| 26 | 360 | 1,082 | 174 | 264 | 485.1 |
| 27 | 160 | 981.5 | 175 | 22.4 | 10.1 |
| 28 | 394 | 76.5 | 176 | 69.9 | 290.3 |
| 29 | 160 | 146.5 | 177 | 53.3 | 59.4 |
| 30 | 58 | 576.3 | 178 | 3,654 | 2,207 |
| 31 | 203 | 115.8 | 179 | 10.9 | 51.6 |
| 32 | 62 | 135.3 | 180 | 3,681 | |
| 33 | 2,036 | | 181 | 192.6 | 153.9 |
| 34 | 8,400 | | 182 | 26.9 | 52.9 |
| 35 | 293 | 2,023 | 183 | 767.7 | 370.4 |
| 36 | 2,429 | | 184 | 150.9 | 57.3 |
| 37 | 112 | 1,483 | 185 | 118.8 | 275.2 |
| 38 | 46 | 155.6 | 186 | 6.3 | 15.1 |
| 39 | 22.6 | 56.4 | 187 | 248.6 | 245.7 |
| 40 | 25 | 143.2 | 188 | 44.9 | 450.1 |
| 41 | 100 | 494.8 | 189 | 15.3 | 124.5 |
| 42 | 3,171 | | 190 | 87.1 | 139.3 |
| 43 | 1,464 | 2,382 | 191 | 76.9 | 168.4 |
| 44 | 47 | 231.3 | 192 | 1,665 | 1,242 |
| 45 | 5,126 | | 193 | 50.7 | 140.2 |
| 46 | 260 | 532.6 | 194 | 33.8 | 21.0 |
| 47 | 11 | 11.6 | 195 | 128.4 | 383.4 |
| 48 | 161 | 1,270 | 196 | 19.8 | 58.5 |
| 49 | 1,806 | | 197 | 80.6 | 397 |
| 50 | 1,583 | | 198 | 1,127 | 316.5 |
| 51 | 686 | 2,050 | 199 | 570.4 | 2,772 |
| 52 | 1,307 | | 200 | 31.2 | 107.1 |
| 53 | 223 | 734.8 | 201 | 6.5 | 39.5 |
| 54 | 24 | 136.7 | 202 | 346 | 518.9 |
| 55 | 1,052 | 1,824 | 203 | 63.9 | 135.2 |
| 56 | 20 | 23.7 | 204 | 405.8 | 2,240 |
| 57 | 334 | 2,148 | 205 | 246.4 | 193.1 |
| 58 | 595 | 1,441 | 206 | 269.7 | 32.5 |
| 59 | 93 | 34.5 | 207 | 347.2 | 860.8 |
| 60 | 2,012 | | 208 | 717.9 | 2,170 |
| 61 | 857 | 4,962 | 209 | 1,065 | 2,179 |
| 62 | 24 | 211.6 | 210 | 91.6 | 294.5 |
| 63 | 85 | 325.7 | 211 | 465 | 3,685 |
| 64 | 1,816 | | 212 | 686.6 | 1,032 |
| 65 | 713 | 2,028 | 213 | 1,762 | 4,518 |
| 66 | 2,124 | | 214 | 37.3 | 101.8 |
| 67 | 70 | 365 | 215 | 33.6 | 133.8 |
| 68 | 30 | 37.0 | 216 | 23.3 | 58.9 |
| 69 | 120 | 432.3 | 217 | 62.5 | 394 |
| 70 | 26 | 70.6 | 218 | 141.1 | 385.4 |
| 71 | 21 | 217.4 | 219 | 106.8 | 297.1 |
| 72 | 3,627 | | 220 | 169 | 80.6 |
| 73 | 391 | | 221 | 47.8 | 111.4 |
| 74 | 32 | 341.8 | 222 | 34.3 | 66.6 |
| 75 | 33 | 186.3 | 223 | 118 | 606.5 |
| 76 | 90 | 736 | 224 | 47.8 | 85.1 |
| 77 | 490 | | 225 | 1,272 | 2,320 |
| 78 | 532 | | 226 | 50.4 | 225.3 |
| 79 | 79 | 1,301 | 227 | 112 | 167.7 |
| 80 | 157 | 971.1 | 228 | 15.9 | 49.1 |
| 81 | 75 | 701.6 | 229 | 28.6 | 85.1 |
| 82 | 32 | 156.2 | 230 | 4,458 | 9,470 |
| 83 | 22 | 189.5 | 231 | 1,078 | 1,970 |
| 84 | 37 | 94.4 | 232 | 117.1 | 158 |
| 85 | 10,076 | | 233 | 77.8 | 90.1 |
| 86 | 22 | 23.5 | 234 | 339.9 | 207 |
| 87 | 36 | 352.5 | 235 | 229.3 | 214 |
| 88 | 2,059 | | 236 | 244.8 | 537.3 |
| 89 | 18 | 29.7 | 237 | 27.1 | 53.3 |
| 90 | 37 | 48.6 | 238 | 221.9 | 137 |
| 91 | 71 | 43.0 | 239 | 977.4 | 3,213 |
| 92 | 3,038 | | 240 | 200.4 | 263.9 |
| 93 | 830 | | 241 | 2,917 | 5,799 |
| 94 | 40 | 118 | 242 | 250.3 | 651.6 |
| 95 | 4,232 | | 243 | 290 | 316.6 |
| 96 | 2,315 | | 244 | 2,126 | 1,574 |
| 97 | 535 | 1,859 | 245 | 52.4 | 92.5 |
| 98 | 628 | 3,279 | 246 | 257.2 | 1,222 |
| 99 | 278 | 2,477 | 247 | 20.9 | 40.8 |
| 100 | 886 | 2,563 | 248 | 126.5 | 427 |
| 101 | 7.5 | 45.2 | 249 | 226 | 695.1 |
| 102 | 9.5 | 17.3 | 250 | 75.9 | 190 |
| 103 | 2,698 | | | >10,000 | |
| 104 | 12 | 23.6 | | 139.9 | 271.2 |
| 105 | 15 | 55.5 | 251 | 1,514 | 1,510 |
| 106 | 48 | 202.4 | 252 | 828.9 | 1,165 |
| 107 | 25 | 34.5 | 253 | 355 | 330.6 |
| 108 | 5.8 | 42.3 | 254 | 178.1 | 318.4 |
| 109 | 3,453 | | 255 | 165 | 665.8 |
| 110 | 15 | 76.5 | 256 | 50.2 | 82.0 |
| 111 | 55 | 111.5 | 257 | 938 | 2,503 |
| 112 | 41 | 180.7 | 258 | 107.9 | 208.6 |
| 113 | 53 | 456.6 | 259 | 1,020 | 1,973 |
| 114 | 73.9 | 57.2 | 260 | >10,000 | |
| 115 | 258 | 72.4 | 261 | 58 | 143.4 |
| 116 | 69.1 | 415.2 | 262 | 99.4 | 446.7 |
| 117 | 924.9 | 1,063 | 263 | 97.1 | 447.4 |
| 118 | 192 | 581.2 | 264 | 2,540 | |
| 119 | 73 | 143.6 | 265 | 8,070 | |
| 120 | 19 | 72.4 | 266 | 912 | 658.8 |
| 121 | 16 | 32.3 | 267 | >10,000 | |
| 122 | 10 | 13.0 | 268 | 88.1 | 248.1 |
| 123 | 441 | 61.6 | 269 | 416 | 1,068 |
| 124 | 19 | 32.5 | 270 | 200 | 350.3 |
| 125 | 5.6 | 25.8 | 271 | 268 | 1,385 |
| 126 | 72 | 3,955 | 272 | 295 | 650.3 |
| 127 | 698 | | 273 | 34.7 | 66.1 |
| 128 | 89.8 | 277 | 274 | 42.4 | 67.6 |
| 129 | 44 | 119.2 | 275 | 61.3 | 133.4 |
| 130 | 19 | 59.6 | 276 | 573 | 1,464 |
| 131 | 5.5 | 14.9 | 277 | 560 | 1,284 |
| 132 | 66 | 177.9 | 278 | 333 | 122.5 |
| 133 | 320 | | 279 | 64.8 | 69.1 |
| 134 | 311 | | 280 | 17.3 | 48.1 |
| 135 | 186 | 244.8 | 281 | 16.5 | 67.4 |
| 136 | 108 | 279.1 | 282 | 33.7 | 95.3 |
| 137 | 413 | 1,490 | 283 | 32.5 | 34.6 |
| 138 | 53.1 | 101 | 284 | 268.5 | 353.6 |
| 139 | 10.0 | 21.9 | 285 | 71.4 | 90.2 |
| 140 | 277 | | 286 | 143.6 | 429.9 |
| 141 | 16 | 41.1 | 287 | 61.5 | 221.8 |
| 142 | 107 | 197.2 | 288 | 109.6 | 408.4 |
| 143 | 163 | 771.5 | 289 | 24.5 | 81.4 |
| 144 | 48.6 | 181.7 | 290 | 18.6 | 60.3 |
| | | | isomer 1 | | |
| 145 | 139 | 454.1 | 290 | 641.3 | 271.3 |
| | | | isomer 2 | | |
| 146 | 203.6 | 767.7 | 291 | 1,552 | 1,060 |
| 147 | 22.4 | 61.8 | 292 | 287.3 | 206 |
| | | | isomer 1 | | |
| 148 | 62 | 163.8 | 292 | 212.8 | 495.5 |
| | | | isomer 2 | | |
| | | | 293 | >10,000 | |

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A compound of Formula (I): 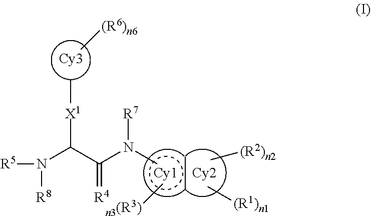
or a N-oxide thereof, or a pharmaceutically acceptable salt thereof, or a stereoisomer thereof, or a deuterated analog thereof, wherein:
Cy1 is a 5-6 membered aromatic ring, said ring comprising 0-3 heteroatoms independently selected from nitrogen, oxygen or sulfur; said ring is optionally substituted with at least one substituent R³;
n3 is independently 0, 1, 2 or 3;
Cy2 is a 4-8 membered saturated or partially or completely unsaturated ring, said ring comprising 0-3 heteroatoms independently selected from nitrogen, oxygen or optionally oxidized sulfur; said ring is substituted with at least one substituent R¹ and/or R²;
n1 is 0 or 1;
n2 is 0, 1 or 2;
n1 and n2 are not 0 at the same time;
Cy1 and Cy2 are fused with each other;
Cy3 is a 3-12 membered saturated or partially or completely unsaturated ring, said ring comprising 0-3 heteroatoms independently selected from nitrogen, oxygen or optionally oxidized sulfur; said ring is optionally substituted with at least one substituent R⁶;
n6 is independently 0, 1, 2, 3, 4 or 5, provided that the valency theory is met;
X¹ is a single bond or —CR⁹R¹⁰—;
R¹ is independently selected from H or 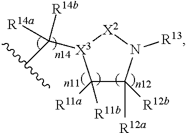
wherein X² is N or C(R¹⁵);
X³ is —C(O)—, —S(O)— or —S(O)₂—;
n11 and n12 are each independently 1, 2 or 3;
n14 is 0 or 1;
R¹⁵ is selected from hydrogen, halogen, —C₁₋₈alkyl, —C₃-C₈cycloalkyl, 3- to 8-membered heterocyclyl, —CN, —OR^{15a}, —NR^{15a}R^{15b}, or —NR^{15a}COR^{15b}; each of —C₁₋₈alkyl, C₃-C₈cycloalkyl and 3- to 8-membered heterocyclyl is optionally substituted with at least one substituent R^{15c},
R^{15a} and R^{15b} are each independently selected from hydrogen, —C₁₋₈alkyl, —C₁₋₈haloalkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl, each of —C₁₋₈alkyl, —C₁₋₈haloalkyl, —C₂₋₈alkenyl, —C₂₋₈alkynyl, C₁₋₈alkoxy-C₁₋₈ alkyl-, C₃-C₈cycloalkyl, 3- to 8-membered heterocyclyl, C₆-C₁₂aryl and 5- to 12-membered heteroaryl is optionally substituted with at least one substituent R^{15d};
R^{15c} and R^{15d}, at each occurrence, are each independently halogen, —OH, —CN, oxo, —C₁₋₈alkyl, —C₁₋₈ haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
R^{11a}, R^{11b}, R^{12a} and R^{12b} are each independently selected from hydrogen, halogen, —C₁₋₈alkyl, —C₂₋₈alkenyl, —C₂₋₈-alkynyl, —C₃-C₈cycloalkyl, 3- to 8-membered heterocyclyl, C₆-C₁₂aryl, 5- to 12-membered heteroaryl, —CN, —OR^{11c}, —COR^{11c}, —CO₂R^{11c}, —CONR^{11c}R^{11d}, —NR^{11c}R^{11d} or —NR^{11c}COR^{11d}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R¹¹°, or
(R^{11a} and R^{11b}), (R^{12a} and R^{12b}), (R^{11a} and R^{12a}), (R^{11a} and R^{12b}), (R^{11b} and R^{12a}) or (R^{11b} and R^{12b}) together with the atom(s) to which they are attached, form a 3- to 8-membered unsaturated or saturated ring, said ring comprising 0-3 heteroatoms independently selected from nitrogen, oxygen or sulfur; said ring is optionally substituted with at least one substituent R^{11f};
R^{11e}, and R^{11f}, at each occurrence, are each independently hydrogen, halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo (═O), —OR^{11g}, thioxo (═S), —SR^{11g}, —CN, —SO₂R^{11g}, —SO₂NR^{11g}R^{11h}, —COR^{11g}, —CO₂R^{11h}, —CONR^{11g}R^{11h}, —NR^{11g}R^{11h}, —NR^{11g}COR^{11h}, —NR^{11g}CO₂R^{11h} or —NR^{11g}SO₂R^{11h}; each of —C₁₋₈alkyl, —C₁₋₈alkoxy, C₃-C₈cycloalkyl, 3- to 8-membered heterocyclyl, C₆-C₁₂aryl or 5- to 12-membered heteroaryl is optionally substituted with at least one substituent R¹¹ⁱ;
R^{11c}, R^{11d}, R^{11g} and R^{11h} are each independently selected from hydrogen, —C₁₋₈alkyl, —C₁₋₈haloalkyl, —C₂₋₈alkenyl, —C₂₋₈alkynyl, C₁₋₈alkoxy-C₁₋₈alkyl-, C₃-C₈cycloalkyl, 3- to 8-membered heterocyclyl, C₆-C₁₂aryl or 5- to 12-membered heteroaryl; each of —C₁₋₈alkyl, —C₁₋₈haloalkyl, —C₂₋₈alkenyl, —C₂₋₈alkynyl, C₁₋₈ alkoxy-C₁₋₈alkyl-, C₃-C₈cycloalkyl, 3- to 8-membered heterocyclyl, C₆-C₁₂aryl and 5- to 12-membered heteroaryl is optionally substituted with at least one substituent R^{11j};
at each occurrence, R¹¹ⁱ and R^{11j} are each independently halogen, —OH, —CN, oxo, —C₁₋₈alkyl, —C₁₋₈haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl; each of —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl is optionally substituted with at least one substituent halogen, —OH, —CN, oxo, —C₁₋₈alkyl, —C₁₋₈haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
R¹³, R^{14a} and R^{14b} are each independently selected from hydrogen, —C₁₋₈alkyl, —C₃-C₈cycloalkyl or 3- to 8-membered heterocyclyl; each of —C₁₋₈alkyl, —C₃-C₈cycloalkyl and 3- to 8-membered heterocyclyl is optionally substituted with at least one substituent R^{13a};
R^{13a}, at each occurrence, is independently halogen, —OH, —CN, oxo, —C₁₋₈alkyl, —C₁₋₈haloalkyl, —C₁₋₈alkoxy, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
R² is independently selected from H, —C₁₋₈alkyl, —C₂₋₈alkenyl, —C₂₋₈alkynyl, —C₃-C₈cycloalkyl, C₆-C₁₂aryl, 5- to 12-membered heteroaryl, —CN, —COR^{2a}, —CO₂R^{2a}, —CONR^{2a}R^{2b}, —NR^{2a}R^{2b}, or —NR^{2a}COR^{2b}, each of —C₁₋₈alkyl, —C₂₋₈alkenyl, —C₂₋₈alkynyl, —C₃-C₈cycloalkyl, C₆-C₁₂aryl and 5- to 12-membered heteroaryl is optionally substituted with at least one substituent R^{2c}; or
when geminal or adjacent, two R² together with the carbon atom(s) to which they are attached, form a 3- to 8-membered unsaturated or saturated ring, said ring comprising 0-3 heteroatoms independently selected from nitrogen, oxygen or sulfur; said ring is optionally substituted with at least one substituent R^{2c};
R^{2a} and R^{2b} are each independently selected from hydrogen, —C₁₋₈alkyl, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{2d}; or
R^{2a} and R^{2b} together with the nitrogen atom to which they are attached, form a 3- to 8-membered unsaturated or saturated ring, said ring comprising 0-3 heteroatoms independently selected from nitrogen, oxygen or sulfur; said ring is optionally substituted with at least one substituent R^{2d};
R^{2c} and R^{2d}, at each occurrence, are each independently hydrogen, halogen, —C₁₋₈alkyl, —C₃-C₈cycloalkyl, 3- to 8-membered heterocyclyl, C₆-C₁₂aryl, 5- to 12-membered heteroaryl, oxo (═O), —OR², —SR^{2e}, —CN, —COR^{2e}, —CO₂R^{2e}, —CONR^{2e}R², —NR^{2e}R², —NR^{2e}COR^{2f} or —NR^{2e}CO₂R^{2f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{2g};
R^{2e} and R^{2f} are each independently selected from hydrogen, —C₁₋₈alkyl, —C₁₋₈alkoxy, C₁₋₈alkoxy-C₁₋₈ alkyl-, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₁₋₈alkoxy, C₁₋₈alkoxy-C₁₋₈alkyl-, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{2h};
R^{2g} and R^{2h}, at each occurrence, are each independently halogen, —OH, —C₁₋₈alkyl, —C₁₋₈haloalkyl, —C₁₋₈alkoxy, C₁₋₈alkoxy-C₁₋₈alkyl-, —C₂₋₈alkenyl, —C₂₋₈alkynyl, —C₃-C₈cycloalkyl, —C₃-C₈halocycloalkyl, 3- to 8-membered heterocyclyl, —C₆-C₁₂aryl, or 5- to 12-membered heteroaryl;
when n1 and n2 are both 1, R¹ and R² are geminal or adjacent;
R³ is independently selected from hydrogen, halogen, —C₁₋₈alkyl, —C₁₋₈alkoxy, —C₃-C₈cycloalkyl, or —CN, wherein each of —C₁₋₈alkyl, —C₁₋₈alkoxy and —C₃-C₈cycloalkyl is optionally substituted with at least one substituent selected from halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁴ is oxo (═O) or thioxo (═S); or
R³ and R⁴ together with the atoms to which they are attached, form a 5- to 6-membered aromatic ring, said ring comprising 1-3 heteroatoms independently selected from nitrogen, oxygen or sulfur; said ring is optionally substituted with at least one substituent R^{3a};
R^{3a} is independently selected from hydrogen, halogen, —C₁₋₈alkyl, —C₁₋₈alkoxy, —C₃-C₈cycloalkyl, or —CN, wherein each of —C₁₋₈alkyl, —C₁₋₈alkoxy and —C₃-C₈cycloalkyl is optionally substituted with at least one substituent selected from halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁵ is C₆-C₁₂aryl, 5- to 12-membered heteroaryl, —COR^{5a}, —CO₂R^{5a}, —CONR^{5a}R^{5b}, wherein each of C₆-C₁₂aryl and 5- to 12-membered heteroaryl is optionally substituted with at least one substituent R^{5c};
R^{5a} and R^{5b} are each independently selected from —C₁₋₈alkyl, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₂₋₈-alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{5d}; or
R^{5a} and R^{5b} together with the atom to which they are attached, form a 3- to 8-membered unsaturated or saturated ring, said ring comprising 0-3 heteroatoms independently selected from nitrogen, oxygen or sulfur; said ring is optionally substituted with at least one substituent R^{5d};
R^{5c} and R^{5d}, at each occurrence, are each independently selected from hydrogen, halogen, —C₁₋₈alkyl, —C₃-C₈cycloalkyl, 3- to 8-membered heterocyclyl, C₆-C₁₂aryl, 5- to 12-membered heteroaryl, oxo (═O), —OR^{5e}, —SR^{5e}, —CN, —COR^{5e}, —CO₂R^{5e}, —CONR^{5e}R^{5d}, —NR^{5e}R^{5f}, —NR^{5e}COR^{5f} or —NR^{5e}CO₂R^{5f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{5g};
R^{5e} and R^{5f}are each independently selected from hydrogen, —C₁₋₈alkyl, —C₁₋₈alkoxy, C₁₋₈alkoxy-C₁₋₈ alkyl-, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; each of —C₁₋₈alkyl, —C₁₋₈alkoxy, C₁₋₈alkoxy-C₁₋₈alkyl-, —C₂₋₈alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent R^{5h};
R^{5g} and R^{5h} are each independently selected from halogen, —OH, —CN, —C₁₋₈alkyl, —C₁₋₈alkoxy, C₁₋₈ alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁶ is H, halogen, —C₁₋₈alkyl, —C₂₋₈alkenyl, —C₂₋₈alkynyl, —C₃-C₈cycloalkyl, C₆-C₁₂aryl, 5- to 12-membered heteroaryl, —CN, —OR^{6a}, —NR^{6a}R^{6b}, —COR^{6a}, —CO₂R^{6a}, —CONR^{6a}R^{6b}, —COR^{6a} or —NR^{6a}COR^{6b}each of —C₁₋₈alkyl, —C₂₋₈alkenyl, —C₂₋₈alkynyl, —C₃-C₈cycloalkyl, C₆-C₁₂aryl and 5- to 12-membered heteroaryl is optionally substituted with at least one substituent R^{6e};
R^{6a} and R^{6b} are each independently selected from hydrogen, —C₁₋₈alkyl, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl; each of —C₁₋₈alkyl, —C₂₋₈-alkenyl, —C₂₋₈alkynyl, C₃-C₁₂cycloalkyl, 3- to 8-membered heterocyclyl, C₆-C₁₂aryl and 5- to 12-membered heteroaryl is optionally substituted with at least one substituent Rd;
R^{6c}, at each occurrence, is independently hydrogen, halogen, —C₁₋₈alkyl, —C₃-C₈cycloalkyl, 3- to 8-membered heterocyclyl, C₆-C₁₂aryl, 5- to 12-membered heteroaryl, oxo (═O), —OR^{6e}, —SR^{6e}, —CN, —COR^{6e}, —CO₂R^{6e}, —CONR^{6e}R^{6f}, —NR^{6e}R^{6f}—NR^{6e}COR^{6f} or —NR^{5e}CO₂R^{6f}; each of —C₁₋₈alkyl, C₃-C₈cycloalkyl, 3- to 8-membered heterocyclyl, C₆-C₁₂aryl or 5- to 12-membered heteroaryl is optionally substituted with at least one substituent R^{6g};
R^{6e} and R^{6f} are each independently selected from hydrogen, —C₁₋₈alkyl, —C₁₋₈alkoxy, C₁₋₈alkoxy-C₁₋₈ alkyl-, —C₂₋₈alkenyl, —C₂₋₈alkynyl, C₃-C₁₂cycloalkyl, 3- to 8-membered heterocyclyl, C₆-C₁₂aryl or 5- to 12-membered heteroaryl;
R^{6d} and R^{6g} are each independently selected from halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl;
R⁷ and R⁸ are each independently hydrogen;
R⁹ and R¹⁰ are each independently selected from H, halogen, —C₁₋₈alkyl, —C₂₋₈alkenyl, —C₂₋₈alkynyl, —C₃-C₈cycloalkyl, C₆-C₁₂aryl or 5- to 12-membered heteroaryl, each of —C₁₋₈alkyl, —C₂₋₈alkenyl, —C₂₋₈alkynyl, —C₃-C₈cycloalkyl, C₆-C₁₂aryl and 5- to 12-membered heteroaryl is optionally substituted with at least one substituent R^{9a}; and
R^{9a}, at each occurrence, is independently halogen, —OH, —C₁₋₈alkyl, —C₁₋₈alkoxy, C₁₋₈alkoxy-C₁₋₈alkyl-, —C₂₋₈alkenyl, —C₂₋₈alkynyl, —C₃-C₈cycloalkyl, 3- to 8-membered heterocyclyl, —C₆-C₁₂aryl, or 5- to 12-membered heteroaryl.

2. The compound of claim 1, wherein the compound is selected from Formula (IIa)-(IIp): 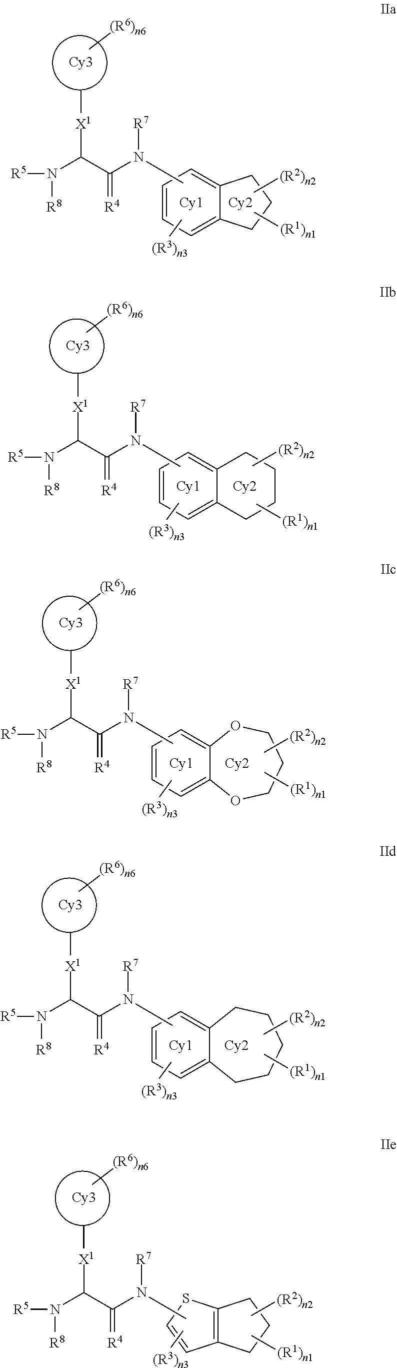 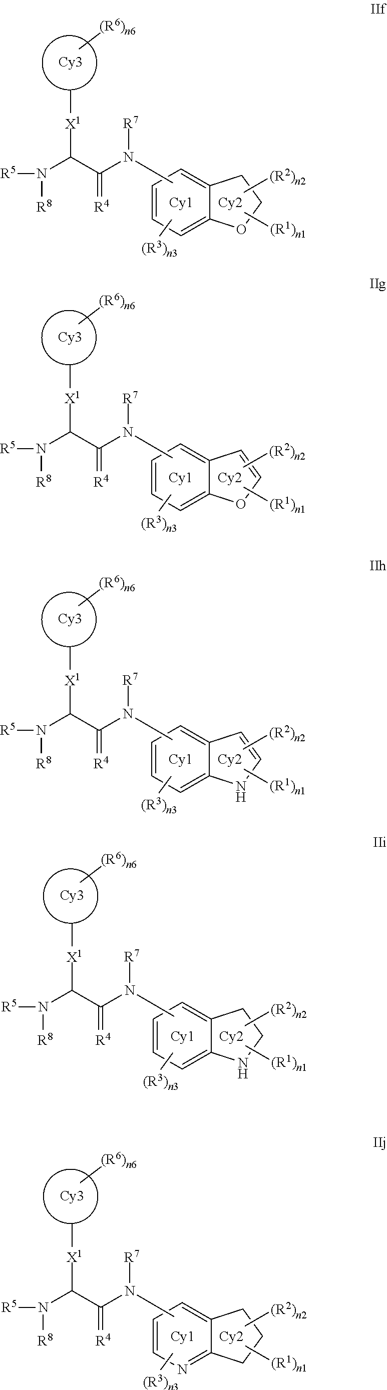 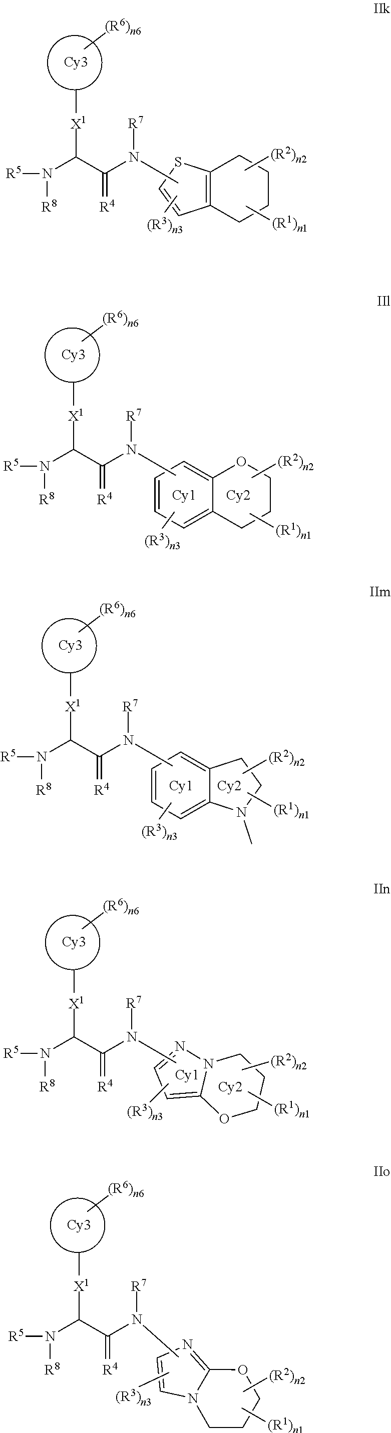 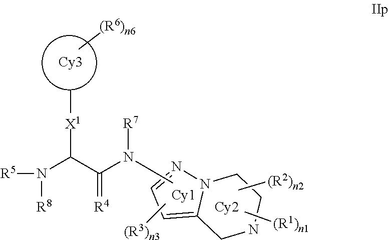
wherein, R¹, R², R³, R⁴, R⁵, R⁶, R⁷, R⁸, Cy3, n1, n2, n3, n6 and X¹ are defined as in claim 1.

3. The compound of claim 1, wherein the compound is selected from Formula (IIf1)-(IIf2): 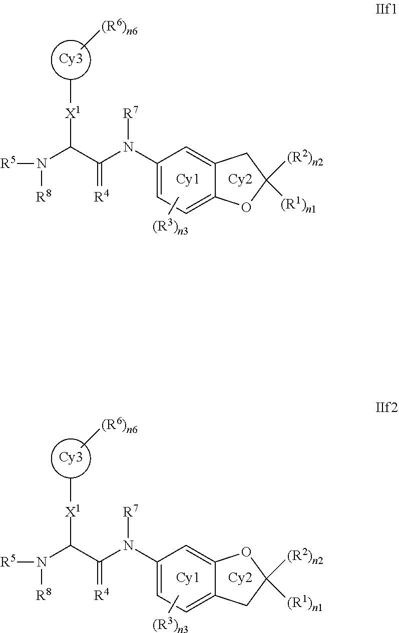
wherein, R¹, R², R³, R⁴, R⁵, R⁶, R⁷, R⁸, Cy3, n1, n2, n3, n6 and X¹ are defined as in claim 1.

4. The compound of claim 1, wherein the compound is selected from Formula (IIIa)-(IIIb): 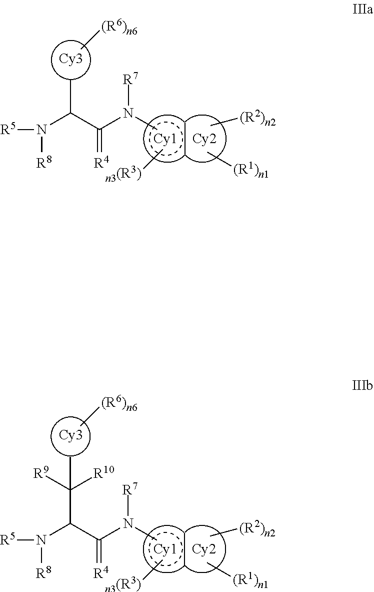
wherein, R¹, R², R³, R⁴, R⁵, R⁶, R⁷, R⁸, Cy1, Cy2, Cy3, n1, n2, n3, n6, R⁹ and R¹⁰ are defined as in claim 1.

5. The compound of claim 1, wherein the compound is selected from Formula (IVa)-(IVb): 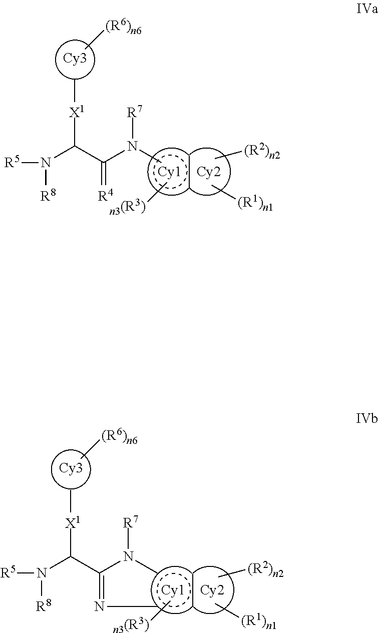
wherein, X¹, R¹, R², R³, R⁵, R⁶, R⁷, R⁸, Cy1, Cy2, Cy3, n1, n2, n3, n6, R⁹ and R¹⁰ are defined as in claim 1.

6. The compound of claim 1, wherein the compound is selected from Formula (Va)-(Vc): 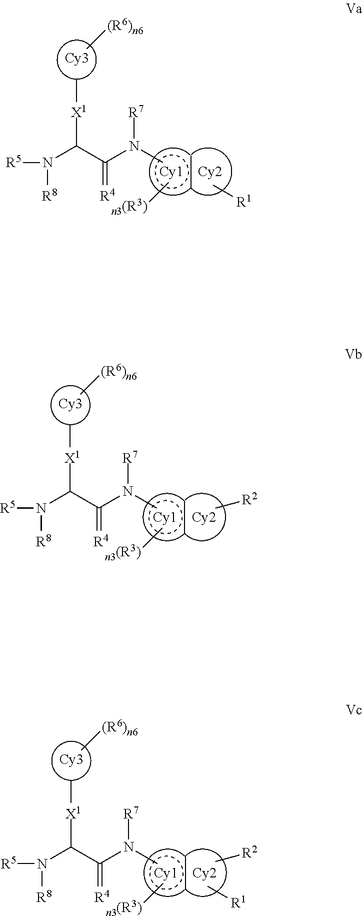
wherein, X¹, R¹, R², R³, R⁴, R⁵, R⁶, R⁷, R⁸, Cy1, Cy2, Cy3, n3, n6, R⁹ and R¹⁰ are defined as in claim 1.

7. The compound claim 1, wherein the compound is VIa, 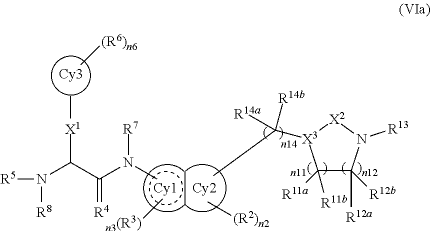
preferably, the compound is selected from VIb and VIc 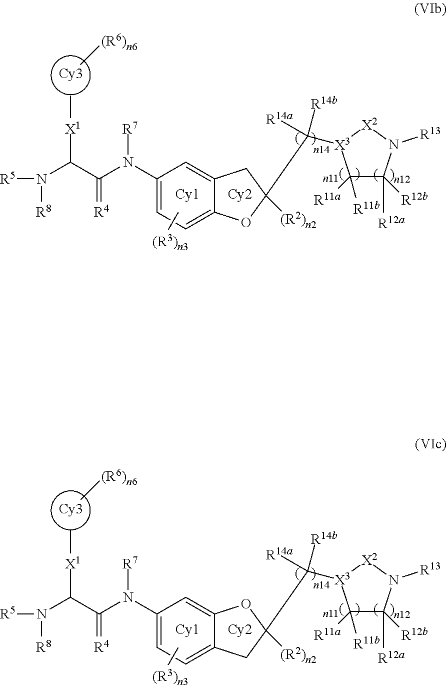
wherein, X¹, X², X³, R², R³, R⁴, R⁵, R⁶, R⁷, R⁸, Cy1, Cy2, Cy3, n2, n3, n6, R^{11a}, R^{11b}, R^{12a}, R^{12b}, R¹³, R^{14a}, R^{14b}, n11, n12 and n14 are defined as in claim 1.

8. The compound claim 1, wherein the compound is VIIa or VIIb, 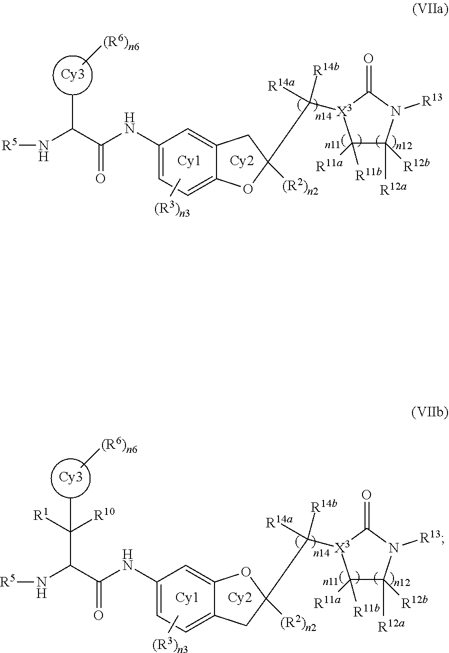
preferably, the compound is selected from VIIc, VIId, VIIe, VIIf, VIIg or VIIh, 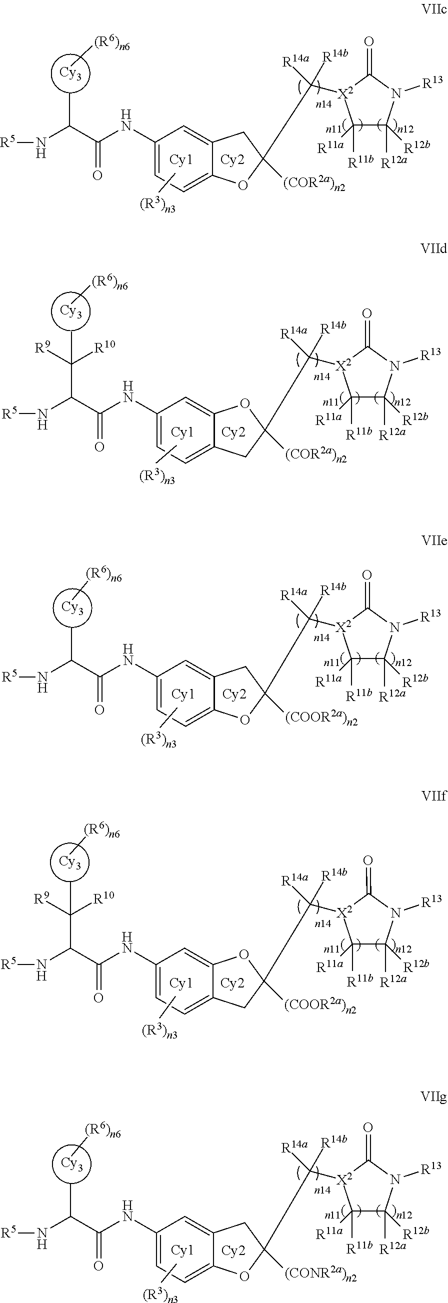 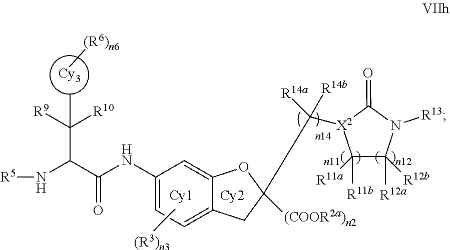
wherein, X², R^{2a}, R³, R⁵, R⁶, R⁹, R¹⁰, Cy1, Cy2, Cy3, n2, n3, n6, R^{11a}, R¹¹ b, R^{12a}, R^{12b}, R¹³, R^{14a}, R^{14b}, n11, n12 and n14 are defined as in claim 1.

9. The compound of any one of the preceding claims, wherein Cy1 is a 5- or 6-membered aromatic ring, said ring comprising 0, 1 or 2 heteroatoms independently selected from nitrogen, oxygen or sulfur; said ring is optionally substituted with 0, 1, 2 or 3 substituents R³;
preferably, Cy1 is phenyl, pyridyl, thiophenyl, imidazolyl, furanyl or pyrazolyl; each of phenyl, pyridyl, thiophenyl, imidazolyl, furanyl or pyrazolyl is optionally substituted with 0, 1 or 2 substituents R³.

10. The compound of any one of the preceding claims, wherein Cy2 a 4-, 5-, 6-, 7- or 8-membered saturated or partially or completely unsaturated ring, said ring comprising 0, 1, 2 or 3 heteroatoms independently selected from nitrogen or oxygen; said ring is substituted with at least one substituent R¹ and/or R²;
preferably, Cy2 a 5-, 6- or 7-membered saturated or partially or completely unsaturated ring, said ring comprising 0, 1 or 2 heteroatoms independently selected from nitrogen or oxygen; said ring is substituted with at least one substituent R¹ and/or R².

11. The compound of any one of the preceding claims, wherein 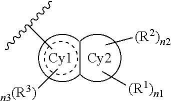
moiety is selected from:

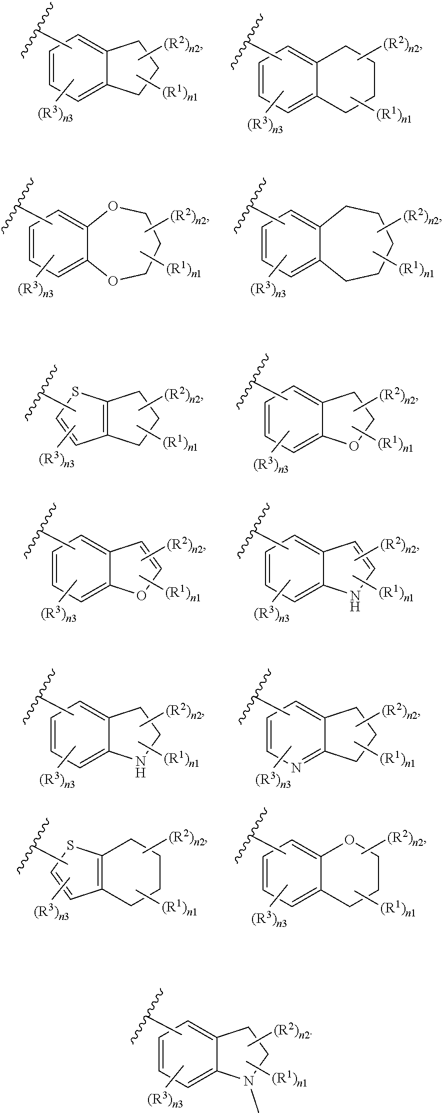

12. The compound of any one of the preceding claims, wherein Cy3 is a 3-, 4-, 5-, 6-, 7-, 8-, 9-, 10-, 11- or 12-membered saturated or partially or completely unsaturated ring, said ring comprising 0, 1, 2 or 3 heteroatoms independently selected from nitrogen, oxygen or optionally oxidized sulfur; said ring is optionally substituted with 0, 1, 2, 3, 4 or 5 substituent R⁶;
preferably, Cy3 is a 3-, 4-, 5-, 6-, 7-, 8-, 9-, 10-, 11- or 12-membered saturated or partially or completely unsaturated ring, said ring is mono-cycle, bi-cycle or tri-cycle comprising 0, 1, 2 or 3 heteroatoms independently selected from nitrogen, oxygen or optionally oxidized sulfur; said ring is optionally substituted with 0, 1, 2, 3, 4 or 5 substituent R⁶;
more preferably, 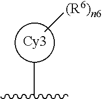
moiety is 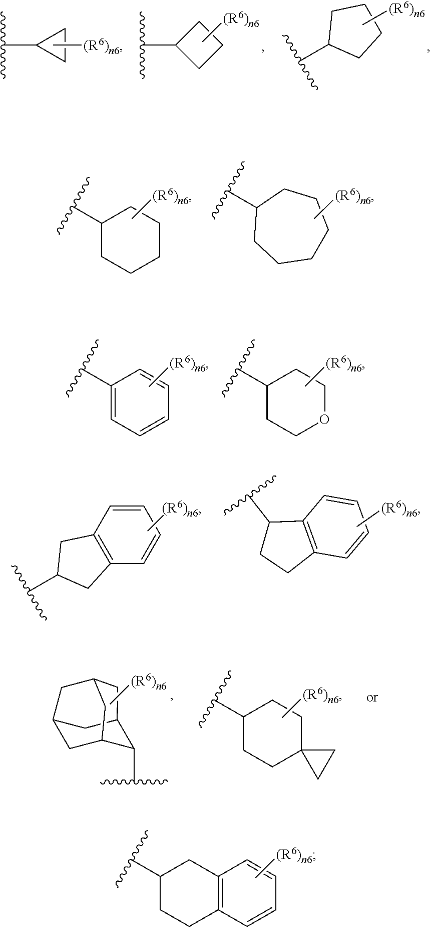
even more preferably, 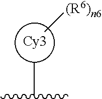
moiety is

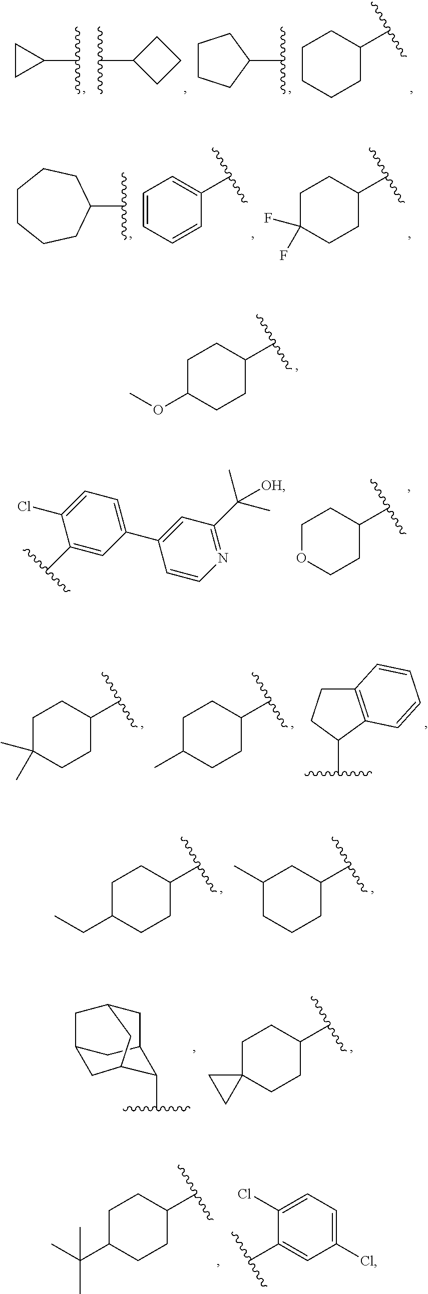

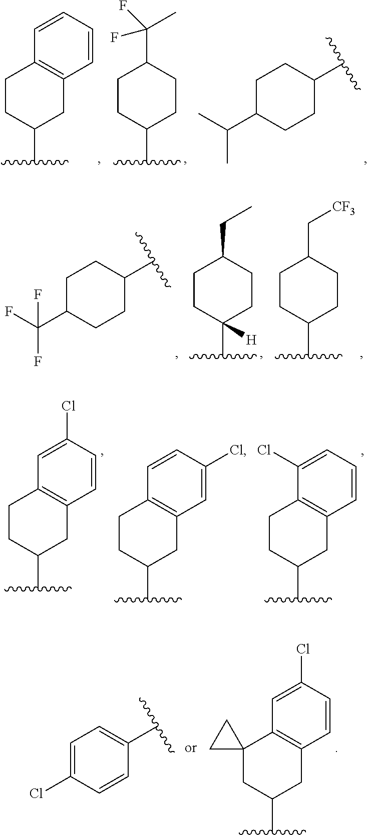

13. The compound of any one of the preceding claims, wherein R¹ is independently selected from 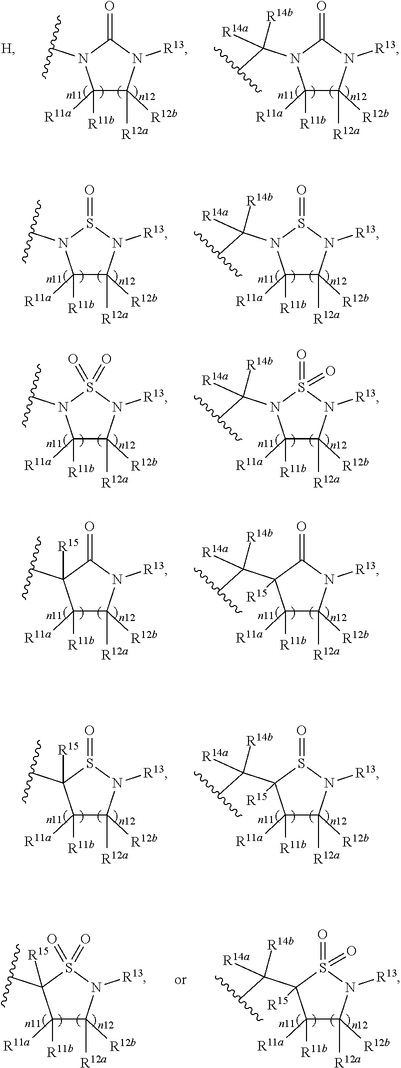
wherein R^{11a}, R^{11b}, R^{12a}, R^{12b}, R¹³, n11 and n12 are defined as in claim 1.

14. The compound of any one of the preceding claims, wherein R¹⁵ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —CN, —OR^{15a}, —NR^{15a}R^{15b}, or —NR^{15a}COR^{15b}; each of methyl, ethyl, propyl, butyl, pentyl, hexyl, heptyl, octyl, cyclopropyl, cyclobutyl, cyclopentyl, cyclohexyl, cycloheptyl, cyclooctyl and 3- to 8-membered heterocyclyl is optionally substituted with at least one substituent R^{15e};
R^{15a} and R^{15b} are each independently selected from hydrogen, methyl, ethyl, propyl, butyl, pentyl, hexyl, heptyl, octyl, —C₁₋₈haloalkyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl, each of methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, C₁₋₈ alkoxy-C₁₋₈alkyl-, cyclopropyl, cyclobutyl, cyclopentyl, cyclohexyl, cycloheptyl, cyclooctyl, 3- to 8-membered heterocyclyl, C₆-C₁₂aryl and 5- to 12-membered heteroaryl is optionally substituted with at least one substituent R^{15d};
R^{15c} and R^{15d}, at each occurrence, are each independently —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, phenyl, or 5- to 12-membered heteroaryl.

15. The compound of any one of the preceding claims, wherein R¹⁵ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, —CN, —OH, methoxy, ethoxy, propoxy, butoxy, pentoxy, hexoxy, heptoxy, octoxy;
preferably, R¹⁵ is selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl;
more preferably, R¹⁵ is selected from hydrogen, —F, —Cl, —Br, —I, methyl, ethyl, propyl, butyl, pentyl, hexyl, heptyl, octyl;
even more preferably, R¹⁵ is selected from hydrogen.

16. The compound of any one of the preceding claims, wherein R^{11a}, R^{11b}, R^{12a} and R^{12b} are each independently selected from hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN; each of methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl and pyrazinyl is optionally substituted with at least one substituent R^{11e};
R^{11e} at each occurrence, is independently hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, oxo (═O), —OR^{11g}, thioxo (═S), —SR^{11g}, —CN, —SO₂R^{11g}, —SO₂NR^{11g}R^{11h}, —COR^{11g}, —CO₂R^{11h}, —CONR^{11g}R^{11h}, —NR^{11g}R^{11h}, —NR^{11g}COR^{11h}, —NR^{11g}COR₂R^{11h} or —NR^{11g}SOR₂R^{11h}; each of methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl and pyrazinyl is optionally substituted with at least one substituent R¹¹ⁱ;
R^{11c}, R^{11d}, R^{11g} and R^{11h} are each independently selected from hydrogen, methyl, ethyl, propyl, butyl, pentyl, hexyl, heptyl, octyl, —C₁₋₈haloalkyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, —C₁₋₈ haloalkyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, phenyl and 5- to 12-membered heteroaryl is optionally substituted with at least one substituent R^{11j};
at each occurrence, R¹¹ⁱ and R^{11j} are each independently —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, —C₃-C₈halocycloalkyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl; each of methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, —C₃-C₈halocycloalkyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl is optionally substituted with at least one substituent —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, phenyl, or 5- to 12-membered heteroaryl.

17. The compound of any one of the preceding claims, wherein R^{11a}, R^{11b}, R^{12a} and R^{12b} are each independently selected from hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN; each of methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl and pyrazinyl is optionally substituted with at least one substituent R^{11e};
R^{11e} at each occurrence, is independently hydrogen, —F, —Cl, —Br, —I, methyl, ethyl, propyl, butyl, pentyl, hexyl, heptyl, octyl, vinyl, propenyl, allyl, but-3-en-1-yl, but-2-en-1-yl, but-1-en-1-yl, pent-4-en-1-yl, pent-3-en-1-yl, pent-2-en-1-yl, pent-1-en-1-yl, hex-5-en-1-yl, hex-4-en-1-yl, hex-3-en-1-yl, hex-2-en-1-yl, hex-1-en-1-yl, hept-6-en-yl, hept-5-en-yl, hept-4-en-yl, hept-3-en-yl, hept-2-en-yl, hept-1-en-yl, oct-7-en-yl, oct-6-en-yl, oct-5-en-yl, oct-4-en-yl, oct-3-en-yl, oct-2-en-yl, oct-1-en-yl, ethynyl, prop-2-yn-1-yl, prop-1-yn-1-yl, but-3-yn-1-yl, but-2-yn-1-yl, but-1-yn-1-yl, pent-4-yn-1-yl, pent-3-yn-1-yl, pent-2-yn-1-yl, pent-1-yn-1-yl, hex-5-yn-1-yl, hex-4-yn-1-yl, hex-3-yn-1-yl, hex-2-yn-1-yl, hex-1-yn-1-yl, hept-6-yn-yl, hept-5-yn-yl, hept-4-yn-yl, hept-3-yn-yl, hept-2-yn-yl, hept-1-yn-yl, oct-7-yn-yl, oct-6-yn-yl, oct-5-yn-yl, oct-4-yn-yl, oct-3-yn-yl, oct-2-yn-yl, oct-1-yn-yl, cyclopropyl, cyclobutyl, cyclopentyl, cyclohexyl, cycloheptyl, cyclooctyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —OR^{11g} or —CN;
R^{11g} is independently selected from hydrogen, methyl, ethyl, propyl, butyl, pentyl, hexyl, heptyl, octyl;
preferably, R^{11a}, R^{11b}, R^{12a} and R^{12b} are each independently selected from H, —CH₃, —CF₃, —CH₂CH₃, —CH₂CF₃, n-propyl, iso-propyl, n-butyl, iso-butyl, sec-butyl, tert-butyl, pentyl, cyclopropyl, cyclobutyl, cyclopentyl, cyclohexyl, cycloheptyl, pyrrolidinyl, tetrahydrofuranyl, oxazolidinyl, piperidinyl, piperazinyl, morpholinyl, tetrahydropyranyl, phenyl, benzyl, vinyl, propenyl, allyl, but-3-en-1-yl, ethynyl, prop-2-yn-1-yl, prop-1-yn-1-yl, but-3-yn-1-yl, but-2-yn-1-yl, but-1-yn-1-yl,

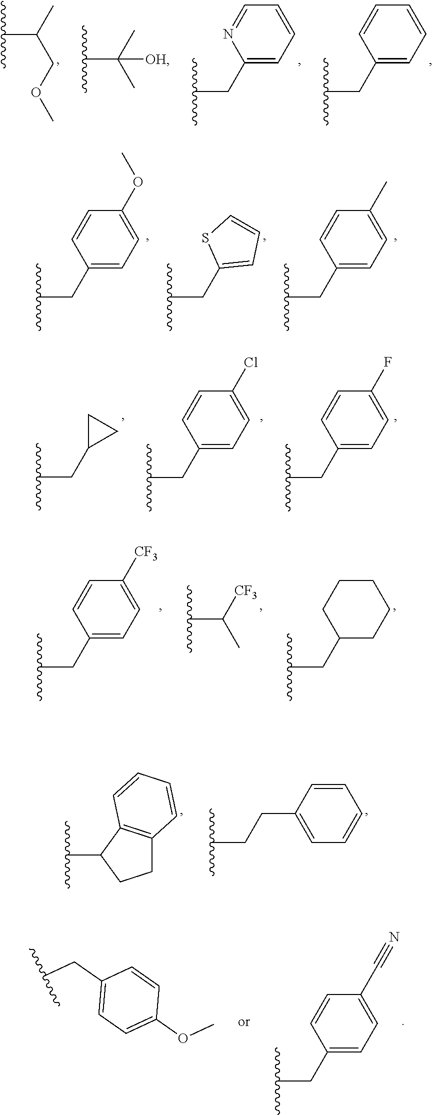

18. The compound of any one of the preceding claims, wherein (R^{11a} and R^{11b}), (R^{12a} and R^{12b}), (R^{11a}and R^{12a}), (R^{11a} and R^{12b}), (R^{11b} and R^{12a}) or (R^{11b} and R^{12b}) together with the carbon atoms to which they are attached, form a 3-, 4-, 5-, 6- or 8-membered unsaturated or saturated ring, said ring comprising 0, 1, 2 or 3 heteroatoms independently selected from nitrogen, oxygen or sulfur; said ring is optionally substituted with at least one substituent R^{11f};
R^{11f}, at each occurrence, is independently hydrogen, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, oxo (═O), —OR^{11g}, thioxo (═S), —SR^{11g}, —CN, —SO₂R^{11g}, —SO₂NR^{11g}R^{11h}, —COR^{11g}, —CO₂R^{11h}, CONR^{11g}R^{11h}, —NR^{11g}R^{11h}, —NR^{11g}COR^{11h}, —NR^{11g}CO₂R^{11h} or —NR^{11g}SO₂R^{11h};
R^{11g} and R^{11h} are each independently selected from hydrogen, methyl, ethyl, propyl, butyl, pentyl, hexyl, heptyl, octyl, —C₂₋₈alkenyl, —C₂₋₈alkynyl, C₁₋₈alkoxy-C₁₋₈alkyl-, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl;
at each occurrence, R¹¹ⁱ is independently —F, —Cl, —Br, —I, —OH, —CN, oxo, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, —C₆-C₁₂aryl, or 5- to 12-membered heteroaryl;
preferably, (R^{11a} and R^{11b}), (R^{12a} and R^{12b}), (R^{11a} and R^{12a}), (R^{11a} and R^{12b}), (R^{11b} and R^{12a}) or (R^{11b} and R^{12b}) together with the carbon atoms to which they are attached, form a 3-, 4-, 5-, 6- or 8-membered unsaturated or saturated ring;
more preferably, (R^{11a} and R^{11b}), (R^{12a} and R^{12b}), (R^{11a} and R^{12a}), (R^{11a} and R^{12b}), (R^{11b} and R^{12a}) or (R^{11b} and R^{12b}) together with the carbon atoms to which they are attached, form a 3-, 4-, 5-, 6- or 8-membered saturated ring;

19. The compound of any one of the preceding claims, wherein R¹³, R^{14a} and R^{14b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl; each of methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl is optionally substituted with at least one substituent R^{13a};
R^{13a}, at each occurrence, is independently halogen, —OH, —CN, oxo, methyl, ethyl, propyl, butyl, pentyl, hexyl, heptyl, octyl, cyclopropyl, cyclobutyl, cyclopentyl, cyclohexyl, cycloheptyl, cyclooctyl, —C₁₋₈haloalkyl, —C₁₋₈alkoxy, —C₂₋₈alkenyl, —C₂₋₈alkynyl, —C₃-C₈halocycloalkyl, 3- to 8-membered heterocyclyl, phenyl, or 5- to 12-membered heteroaryl;
preferably, R¹³, R^{14a} and R^{14b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl;
more preferably, R¹³ is hydrogen, methyl, ethyl, propyl or butyl; R^{14a} and R^{14b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl or 3- to 8-membered heterocyclyl;
even more preferably, R¹³ is hydrogen; R^{14a} and R^{14b} are each independently selected from hydrogen, methyl, ethyl, propyl, butyl, cyclopropyl, cyclobutyl, cyclopentyl.

20. The compound of any one of the preceding claims, wherein R¹ is selected from

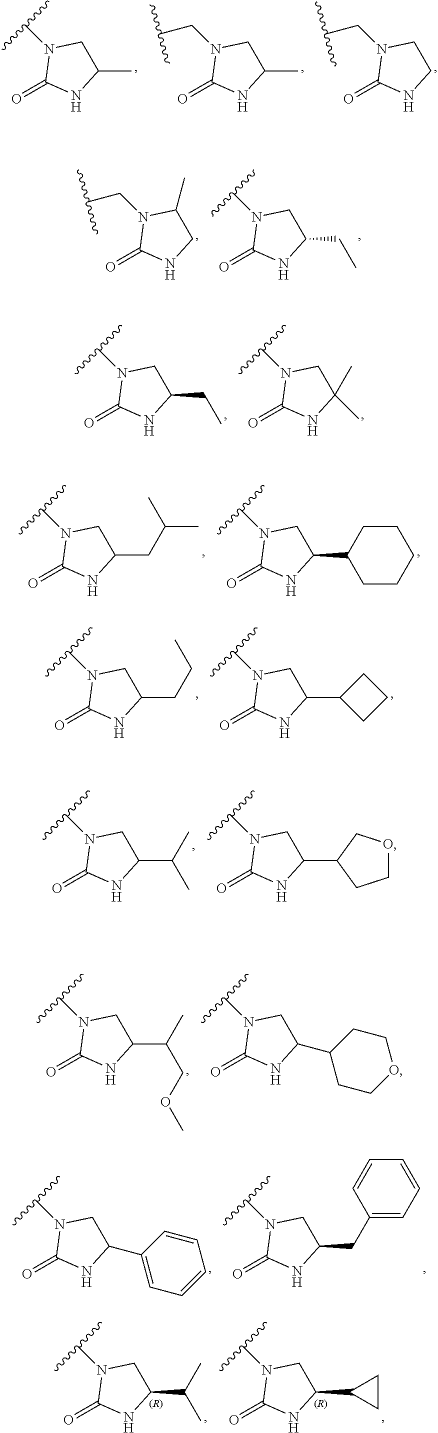

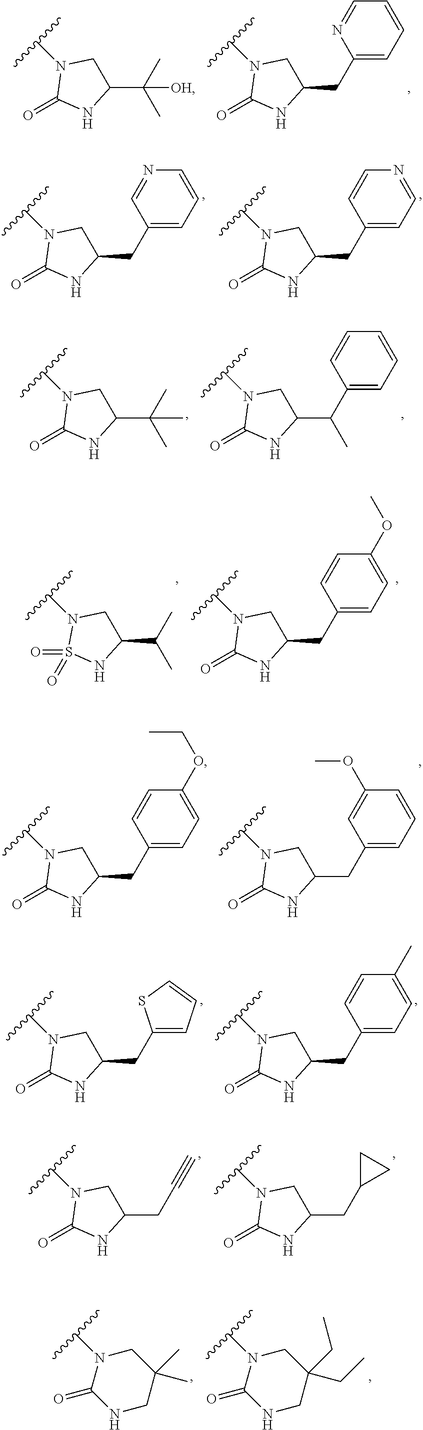

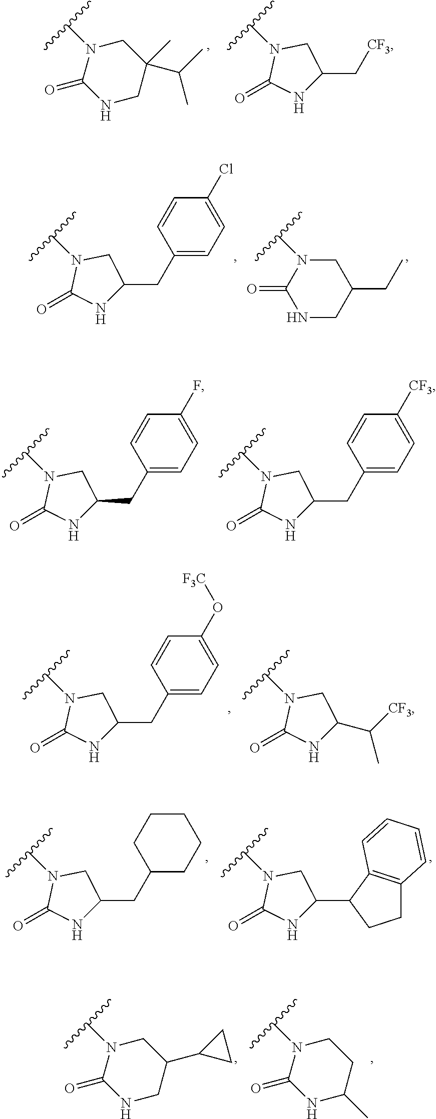

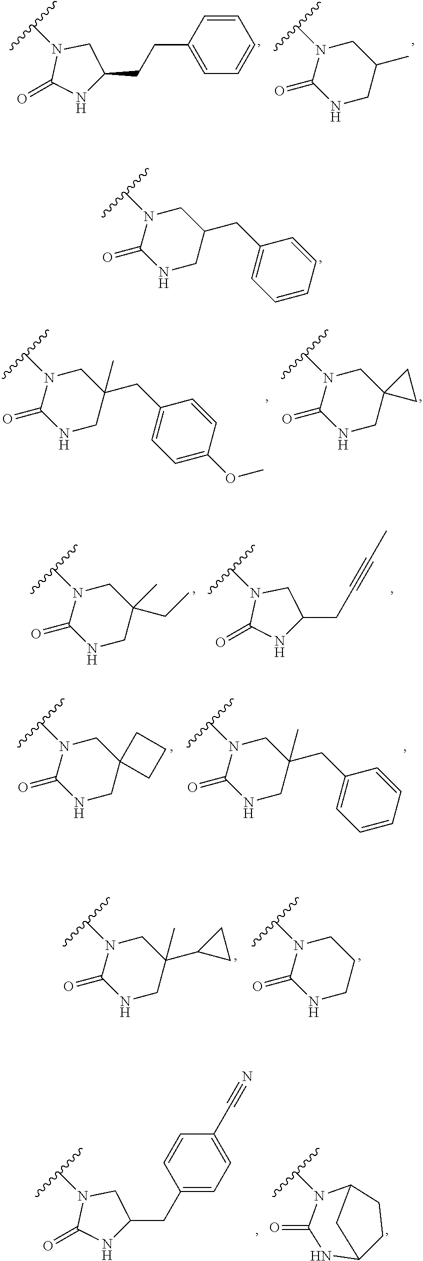

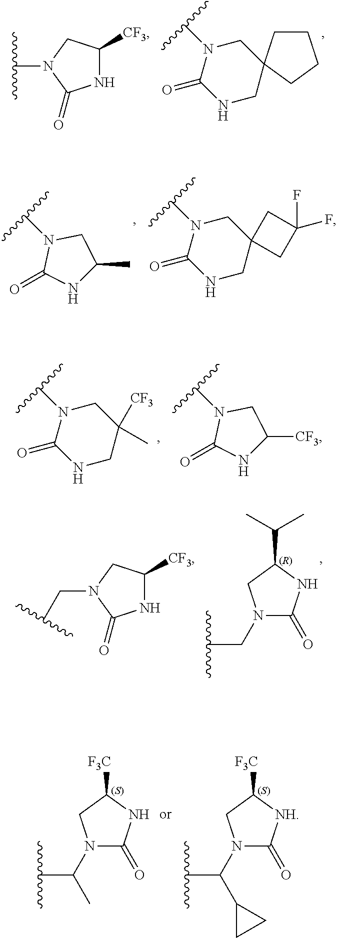

21. The compound of any one of the preceding claims, wherein R² is independently selected from H, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —CN, —COR^{2a}, —CO₂R^{2a}, —CONR^{2a}R^{2b}, —NR^{2a}R^{2b}, —NR^{2a}COR^{2b}, each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl and imidazopyridinyl is optionally substituted with at least one substituent R^{2c};
R^{2a} and R^{2b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2d}; or
R^{2a} and R^{2b} together with the carbon atoms to which they are attached, form a 3-, 4-, 5-, 6-, 7- or 8-membered unsaturated or saturated ring, said ring comprising 0, 1, 2, 3 heteroatoms independently selected from nitrogen, oxygen or sulfur; said ring is optionally substituted with at least one substituent R^{2d};
R^{2c} and R^{2d}, at each occurrence, are each independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, oxo (═O), —OR^{2e}, —SR^{2e}, —CN, —COR^{2e}, —CO₂R^{2e}, —CONR^{2e}R^{2f}, —NR^{2e}R^{2f}, —NR^{2e}COR^{2f} or —NR^{2e}CO₂R^{2f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2g};
R^{2e} and R^{2f}are each independently selected from hydrogen, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈ alkyl-, —C₁₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{2h}
R^{2g} and R^{2h}, at each occurrence, are each independently —F, —Cl, —Br, —I, —OH, methyl, ethyl, propyl, butyl, pentyl, hexyl, heptyl, octyl, —C₁₋₈haloalkyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, —C₃-C₈halocycloalkyl, 3- to 8-membered heterocyclyl, phenyl, or 5- to 12-membered heteroaryl.

22. The compound of any one of the preceding claims, wherein R² is independently selected from H, methyl, ethyl, propyl, butyl, pentyl, hexyl, cyclopropyl, cyclobutyl, cyclopentyl, cyclohexyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —CN, —COR^{2a}, —CO₂R^{2a} or —CONR^{2a}R^{2b}, each of methyl, ethyl, propyl, butyl, pentyl, hexyl, cyclopropyl, cyclobutyl, cyclopentyl, cyclohexyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2c};
R^{2a} and R^{2b} are each independently selected from hydrogen, methyl, ethyl, propyl, butyl, pentyl, hexyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl; each of methyl, ethyl, propyl, butyl, pentyl, hexyl, cyclopropyl, cyclobutyl, cyclopentyl, cyclohexyl, cycloheptyl, cyclooctyl, adamantyl, aziridinyl, azetidinyl, pyrrolidinyl, dihydropyrrolidinyl, tetrahydrofuranyl, dihydrofuranyl, oxazolidinyl, piperidinyl, piperazinyl, morpholinyl, tetrahydropyranyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2d}; or
R^{2a} and R^{2b} together with the carbon atoms to which they are attached, form a 3-, 4-, 5- or 6-membered unsaturated or saturated ring, said ring comprising 0 or 1 heteroatoms independently selected from nitrogen, oxygen or sulfur; said ring is optionally substituted with at least one substituent R^{2d};
R^{2c} and R^{2d}, at each occurrence, are each independently hydrogen (H or D), —F, —Cl, —Br, —I, methyl, ethyl, propyl, butyl, pentyl, hexyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —OR^{2e}, —CN or —NR^{2e}R^{2f}; each of methyl, ethyl, propyl, butyl, pentyl, hexyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl is optionally substituted with at least one substituent R^{2g};
R^{2e} and R^{2f}are each independently selected from hydrogen, methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl or phenyl; each of methyl, ethyl, propyl, butyl, pentyl, hexyl, methoxy, ethoxy, propoxy, butoxy, pentoxy, hexoxy, cyclopropyl, cyclobutyl, cyclopentyl, cyclohexyl or phenyl is optionally substituted with at least one substituent R^{2h}R^{2g} and R^{2h}, at each occurrence, are each independently —F, —Cl, —Br, —I, —OH, methyl, ethyl, propyl, butyl, pentyl, hexyl, methoxy, ethoxy, propoxy, butoxy, pentoxy, hexoxy, cyclopropyl, cyclobutyl, cyclopentyl or cyclohexyl;
preferably, R² is

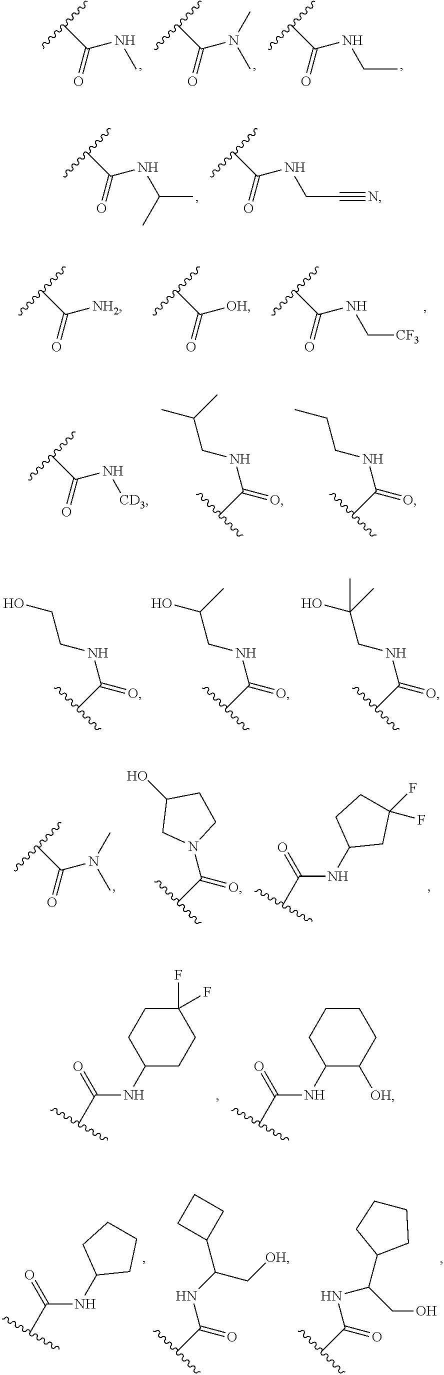

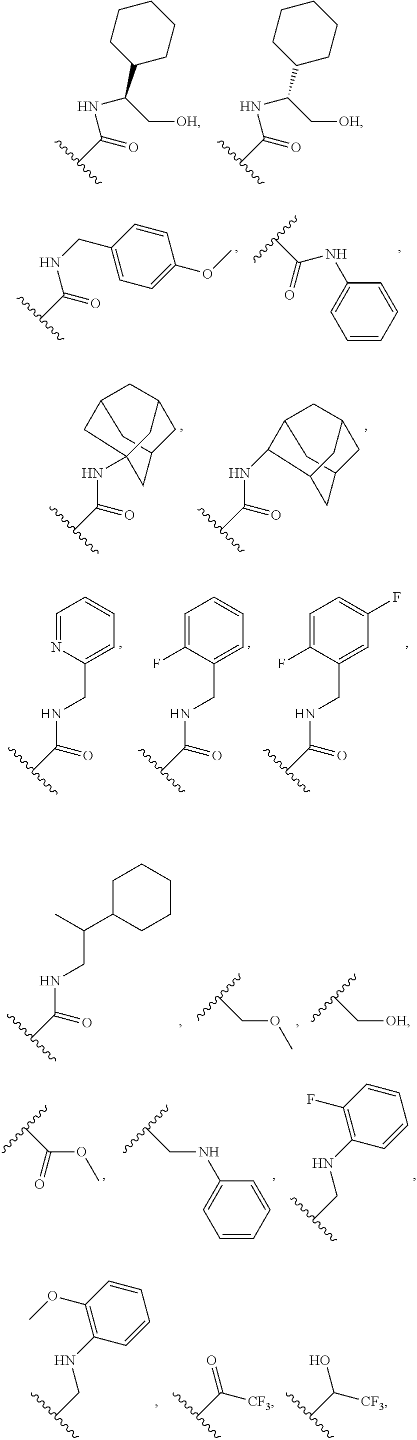

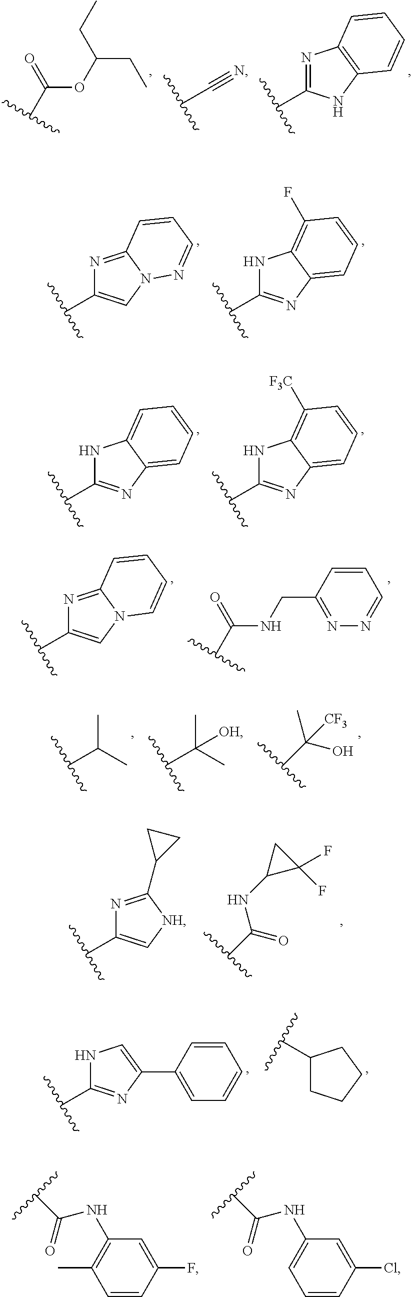

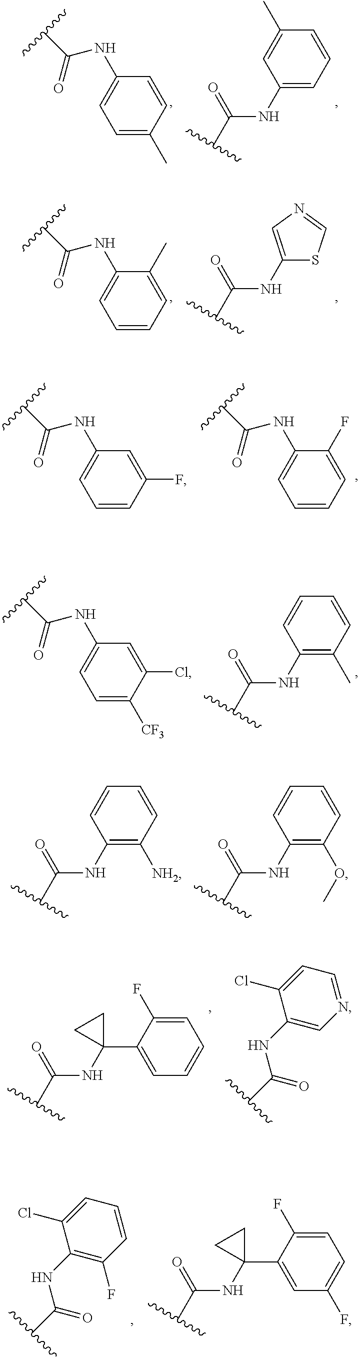

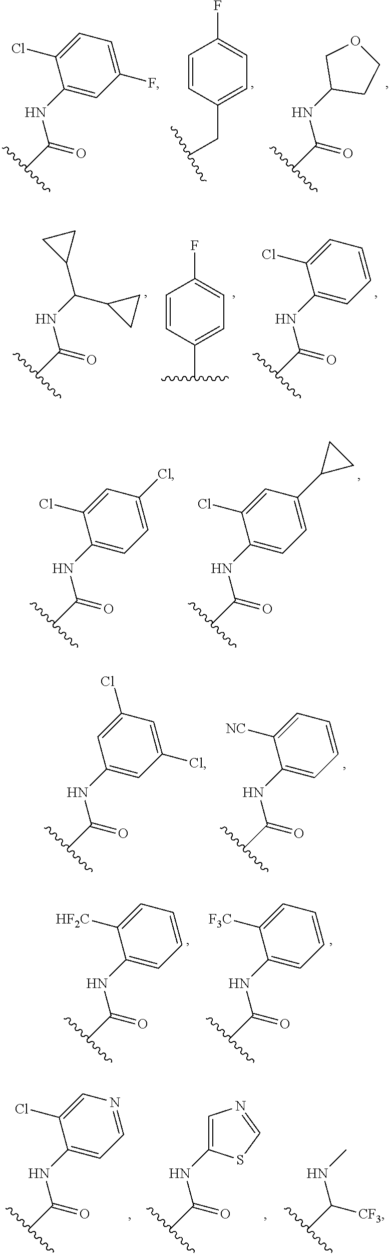

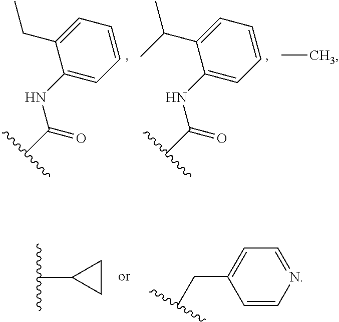

23. The compound of any one of the preceding claims, wherein R³ is independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl or —CN, wherein each of methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl is optionally substituted with at least one substituent selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl;
preferably, R³ is independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl or —CN;
more preferably, R³ is independently selected from hydrogen, —F, —Cl, —Br, —I.

24. The compound of any one of the preceding claims, wherein R⁴ is oxo (═O).

25. The compound of any one of the preceding claims, wherein R³ and R⁴ together with the carbon atoms to which they are attached, form a 5- to 6-membered aromatic ring, said ring comprising 1, 2 or 3 heteroatoms independently selected from nitrogen, oxygen or sulfur; said ring is optionally substituted with at least one substituent R^{3a};
R^{3a} is independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, —CN, wherein each of methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl is optionally substituted with at least one substituent selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, —C₆-C₁₂aryl, or 5- to 12-membered heteroaryl.

26. The compound of any one of the preceding claims, wherein R⁵ is phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl, imidazopyridinyl, —COR^{5a}, —CO₂R^{5a}, or —CONR^{5a}R^{5b}, wherein each of phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5c};
R^{5a} and R^{5b} are each independently selected from methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, oxadiazolyl, triazolyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, oxadiazolyl, triazolyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent Rd; or
R^{5a} and R^{5b} together with the nitrogen atom to which they are attached, form a 3-, 4-, 5-, 6-, 7- or 8-membered unsaturated or saturated ring, said ring comprising 0, 1, 2 or 3 heteroatoms independently selected from nitrogen, oxygen or sulfur; said ring is optionally substituted with at least one substituent R^{5d};
R^{5c} and R^{5d}, at each occurrence, are independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, oxo (═O), —OR^{5e}, —SR^{5e}, —CN, —COR^{5e}, —CO₂R^{5e}, —CONR^{5e}R^{5f}, —NR^{5e}R^{5f}, —NR^{5e}COR^{5f} or —NR^{5e}CO₂R^{5f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl or imidazopyridinyl is optionally substituted with at least one substituent R^{5g};
R^{5e} and R^{5f}are each independently selected from hydrogen, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈ alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5h};
R^{5g} and R^{5h} are each independently selected from halogen, —OH, —CN, methyl, ethyl, propyl, butyl, pentyl, hexyl, heptyl, octyl, methoxy, ethoxy, propoxy, butoxy, pentoxy, hexoxy, heptoxy, octoxy, C₁₋₈ alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

27. The compound of any one of the preceding claims, wherein R⁵ is pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl, wherein each of pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl, imidazopyridinyl, is optionally substituted with at least one substituent R^{5c};
R^{5c}, at each occurrence, is independently selected from hydrogen, —F, —Cl, —Br, —I, methyl, ethyl, propyl, butyl, pentyl, hexyl, cyclopropyl, cyclobutyl, cyclopentyl, cyclohexyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl;
preferably, R⁵ is benzooxazolyl;
more preferably, R⁵ is

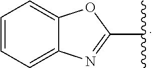 [SMILES: *c1nc2ccccc2o1]

28. The compound of any one of the preceding claims, wherein R⁵ is —COR^{5a}, —CO₂R^{5a} or —CONR^{5a}R^{5b};
wherein R^{5a} and R^{5b} are each independently selected from methyl, ethyl, propyl, butyl, pentyl, hexyl, heptyl, octyl, cyclopropyl, cyclobutyl, cyclopentyl, cyclohexyl, cycloheptyl, cyclooctyl, phenyl, oxadiazolyl, triazolyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5d};
R^{5d}, at each occurrence, is independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl, imidazopyridinyl, —OR^{5e} or —CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzoimidazolyl, imidazopyridazinyl or imidazopyridinyl is optionally substituted with at least one substituent R^{5g};
R^{5e} is selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl; each of methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, 3- to 8-membered heterocyclyl, phenyl, pyrrolyl, pyrazolyl, imidazolyl, oxazolyl, furanyl, thiophenyl, thiazolyl, pyridinyl, pyridazinyl, pyrimidinyl, pyrazinyl, benzooxazolyl, benzoimidazolyl, imidazopyridazinyl or imidazopyridinyl is optionally substituted with at least one substituent R^{5h};
R^{5g} and R^{5h} are each independently selected from halogen, —OH, —CN, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, phenyl or 5- to 12-membered heteroaryl;
preferably, R⁵ 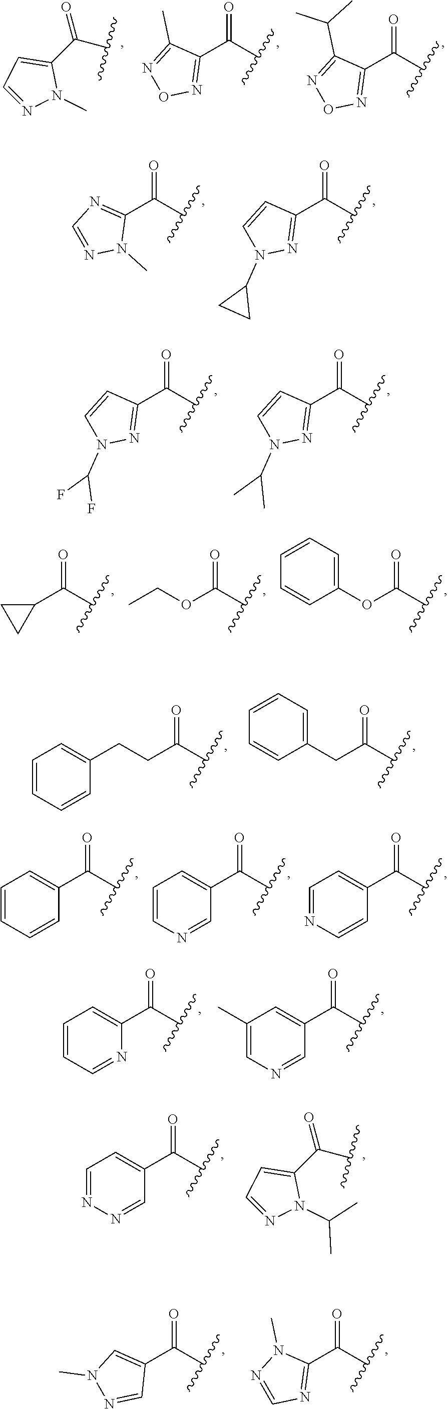 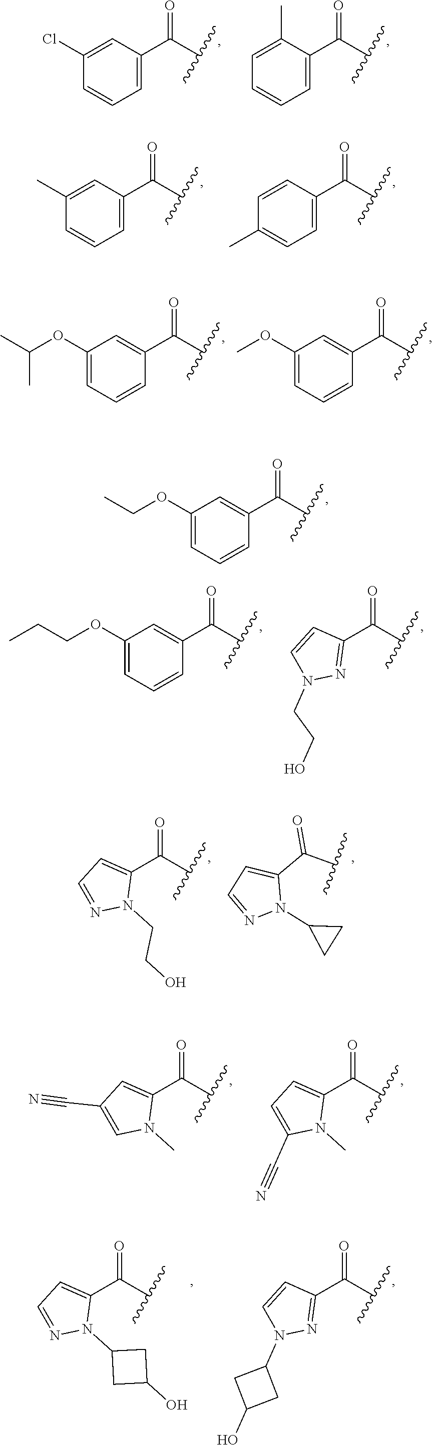 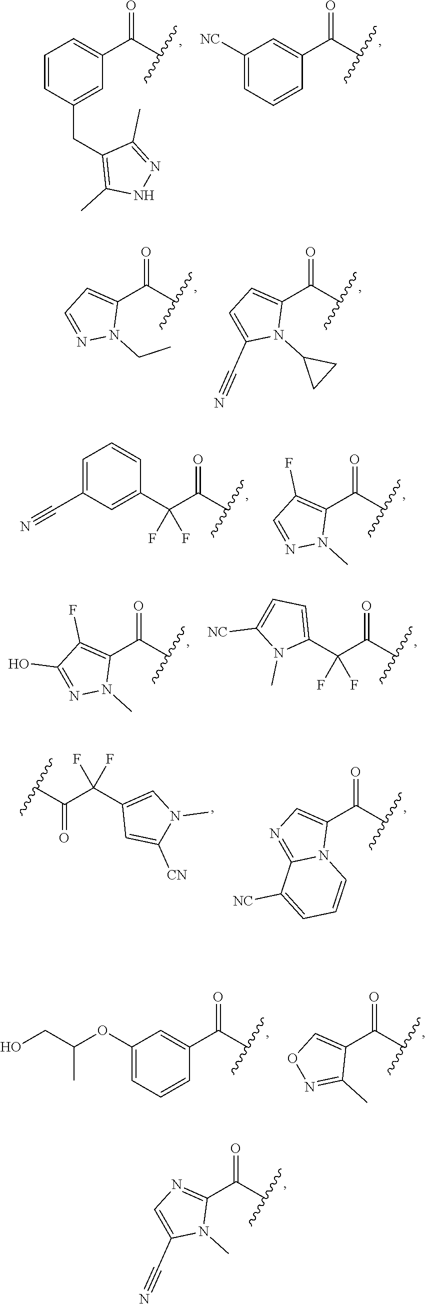
—COCH₃ or —COCF₃.

29. The compound of any one of the preceding claims, wherein R⁶ is H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN, —OR^{6a}, —NR^{6a}R^{6b}, —COR^{6a}, —CO₂R^{6a}, —CONR^{6a}R^{6b}, —COR^{6a} or —NR^{6a}COR^{6b}, each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl is optionally substituted with at least one substituent R^{6C};
R^{6a} and R^{6b} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent Rid;
R^{6c}, at each occurrence, is independently hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —OR^{6e}, —SR^{6e}, —CN, —COR^{6e}, —CO₂R^{6e}, —CONR^{6e}R^{6f}, —NR^{6e}R^{6f}, —NR^{6e}COR^{6f} or —NR^{6e}CO₂R^{6f}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with at least one substituent R^{6g};
R^{6e} and R^{6f} are each independently selected from hydrogen, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈ alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{6d} and R^{6g} are each independently selected from —F, —Cl, —Br, —I, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;

30. The compound of any one of the preceding claims, wherein R⁶ is H, —F, —Cl, —Br, —I, methyl, —CF₃, ethyl, —CH₂CF₃, —CF₂CH₃, propyl, butyl, pentyl, hexyl, heptyl, octyl, cyclopropyl, cyclobutyl, cyclopentyl, cyclohexyl, cycloheptyl, cyclooctyl, phenyl, pyrrolyl, pyrazolyl, imidazolyl, oxazolyl, furanyl, thiophenyl, thiazolyl, pyridinyl, pyridazinyl, pyrimidinyl, pyrazinyl, —CN, methoxy, ethoxy, propoxy, butoxy, pentoxy, hexoxy, heptoxy, octoxy, 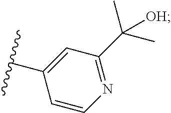 [SMILES: *c1ccnc(C(C)(C)O)c1]
preferably, 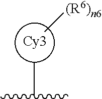
moiety is

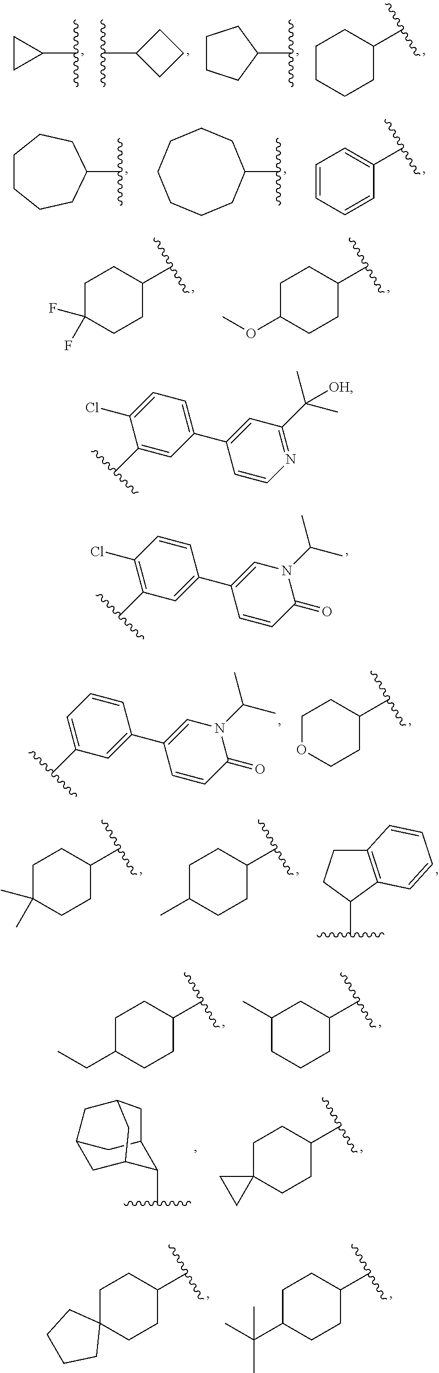

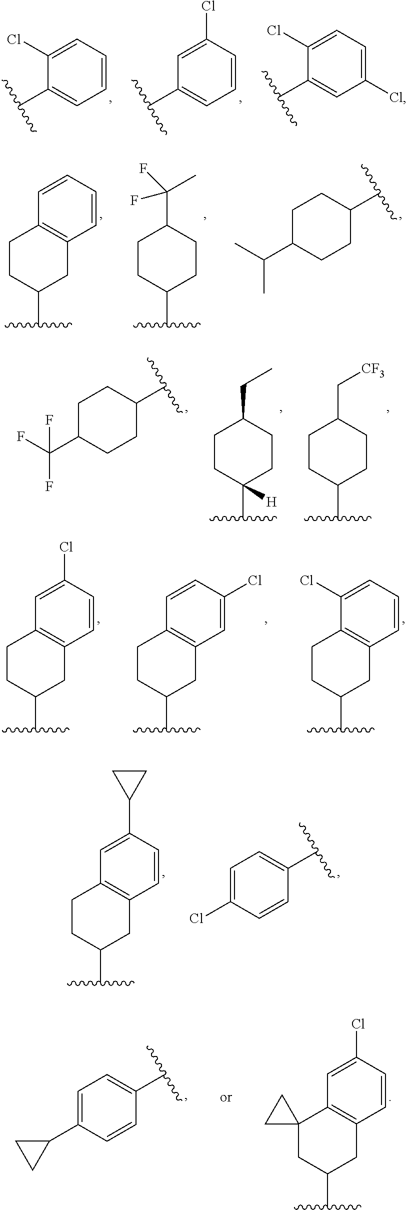

31. The compound of any one of the preceding claims, wherein R⁹ and R¹⁰ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl or 5- to 12-membered heteroaryl, each of methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl or 5- to 12-membered heteroaryl is optionally substituted with at least one substituent R^{9a};
R^{9a}, at each occurrence, is independently halogen, —OH, methyl, ethyl, propyl, butyl, pentyl, hexyl, heptyl, octyl, methoxy, ethoxy, propoxy, butoxy, pentoxy, hexoxy, heptoxy, octoxy, C₁₋₈alkoxy-C₁₋₈alkyl-, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R⁹ and R¹⁰ are each independently selected from H, —F, —Cl, —Br, —I, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, phenyl or 5- to 12-membered heteroaryl;
more preferably, R⁹ and R¹⁰ are each independently selected from H, methyl, ethyl, propyl, butyl, pentyl, cyclopropyl, cyclobutyl, cyclopentyl;
more preferably, R⁹ and R¹⁰ are each independently selected from H, cyclopropyl.

32. The compound of any one of the preceding claims, wherein the compound is selected from

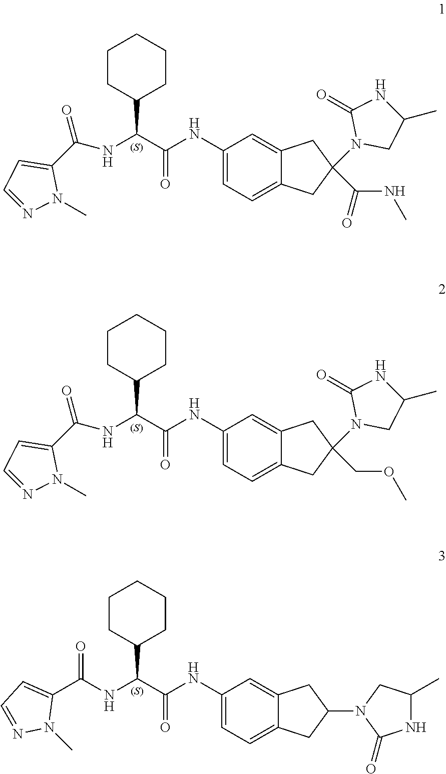

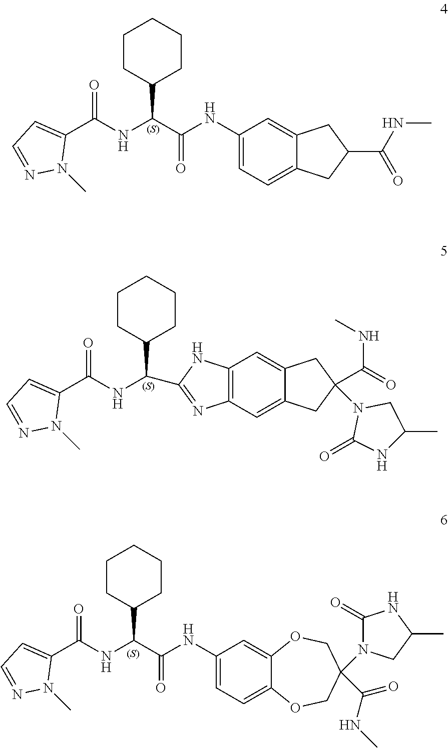

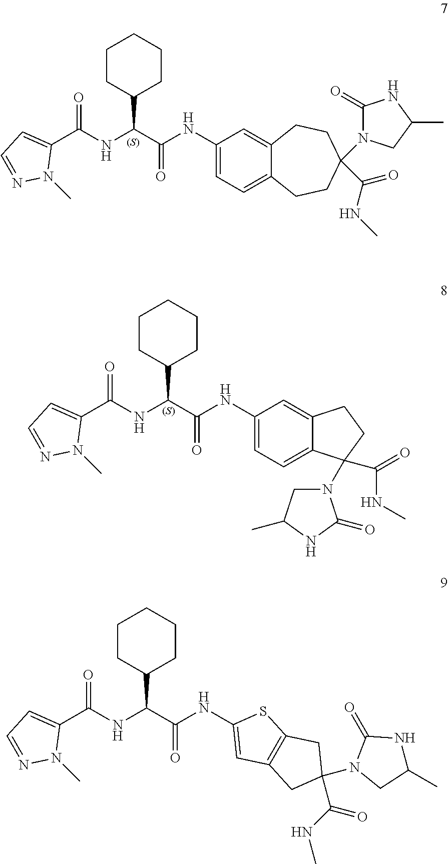

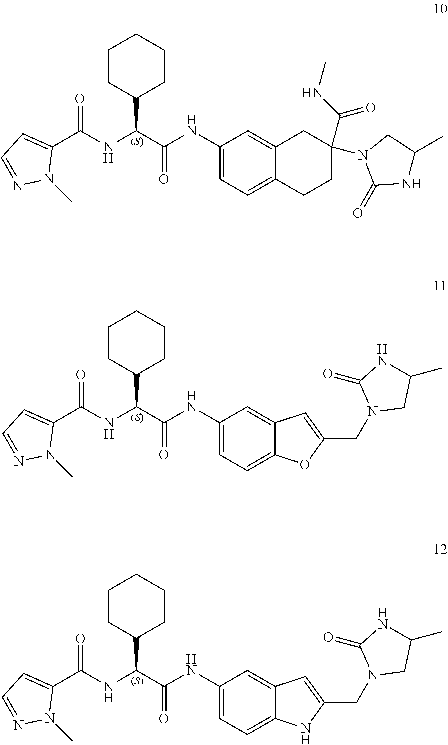

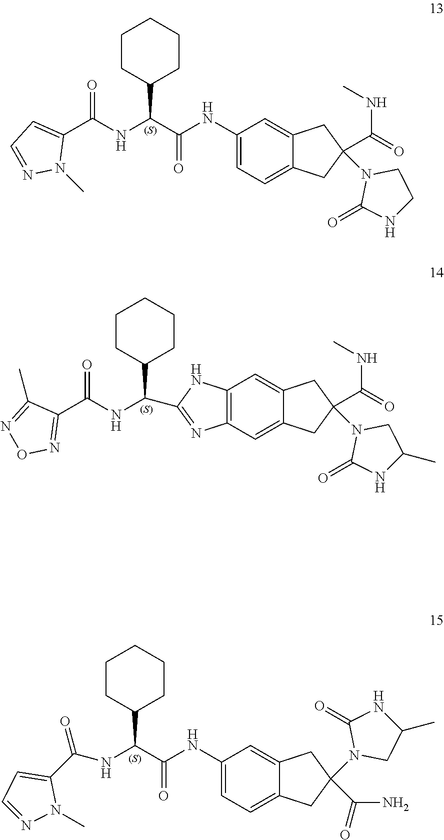

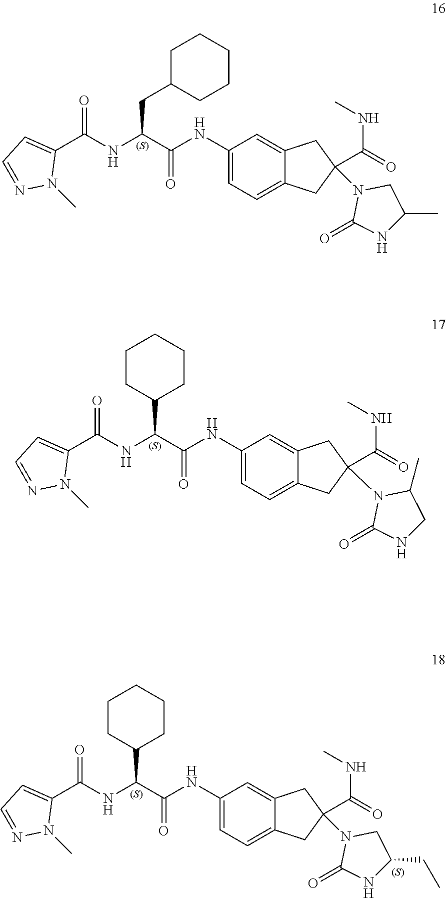

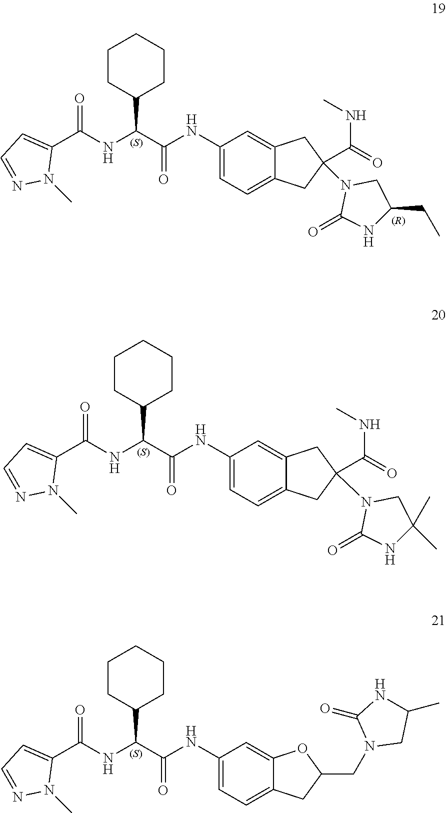

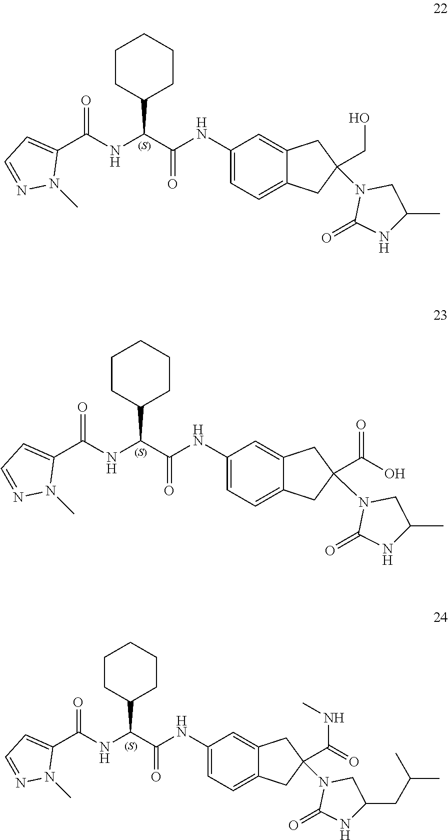

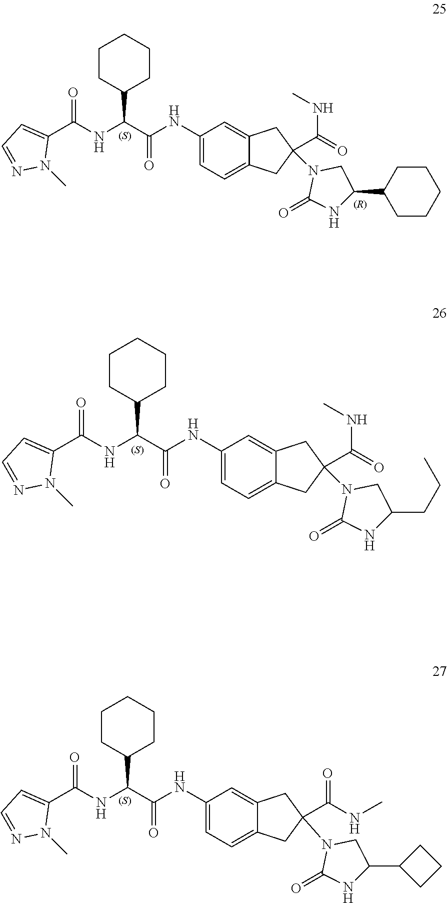

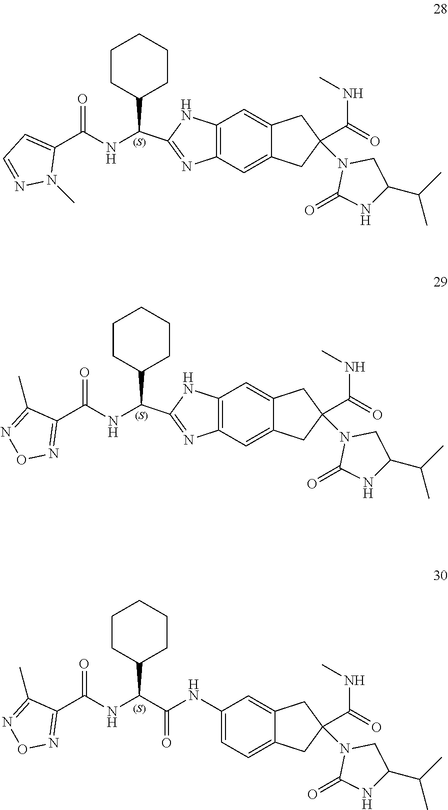

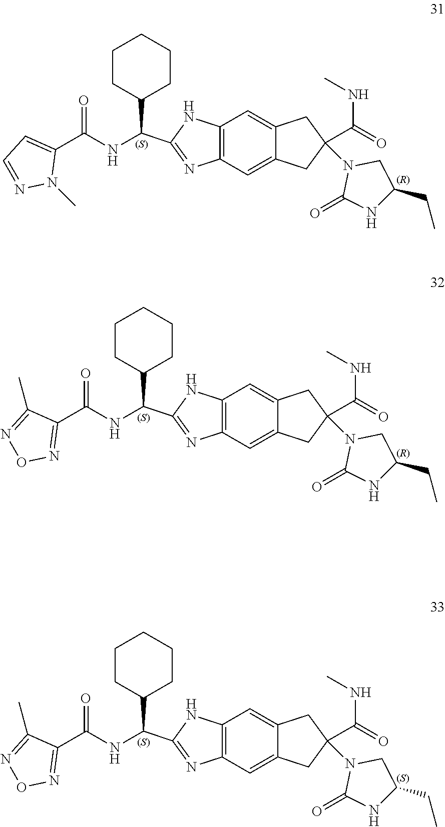

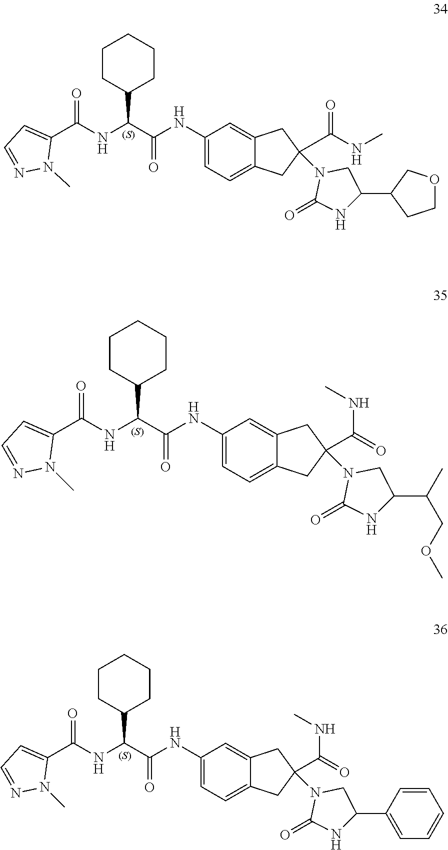

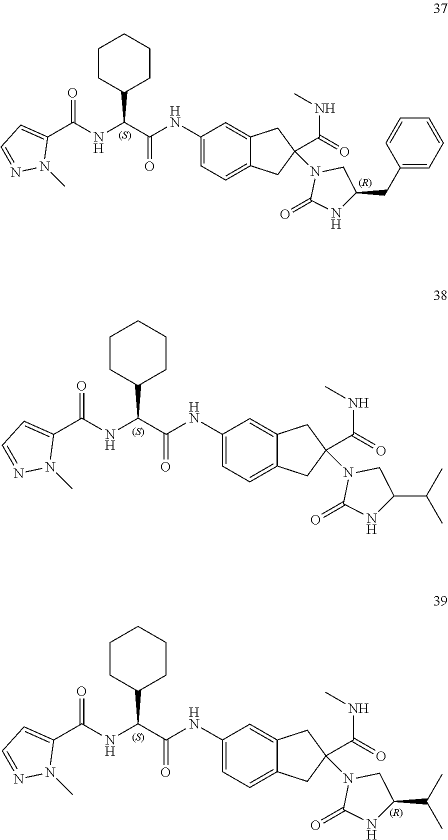

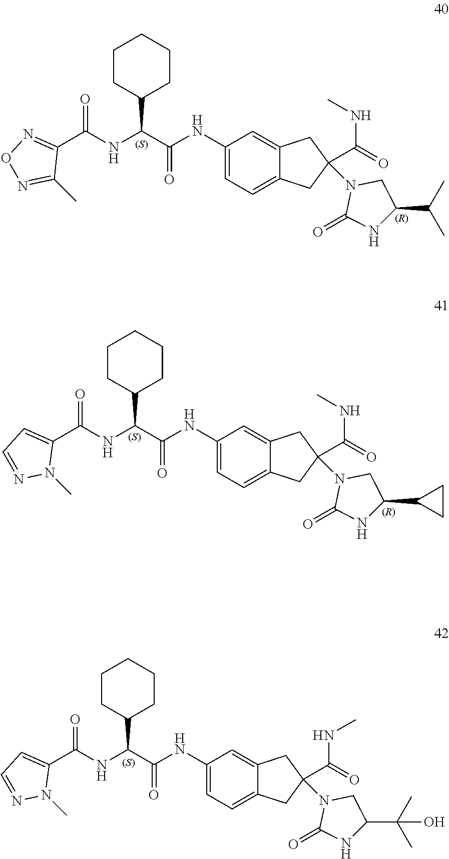

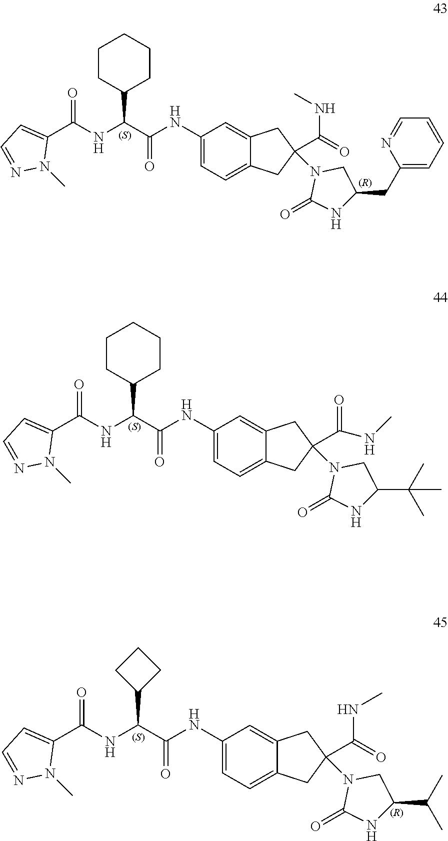

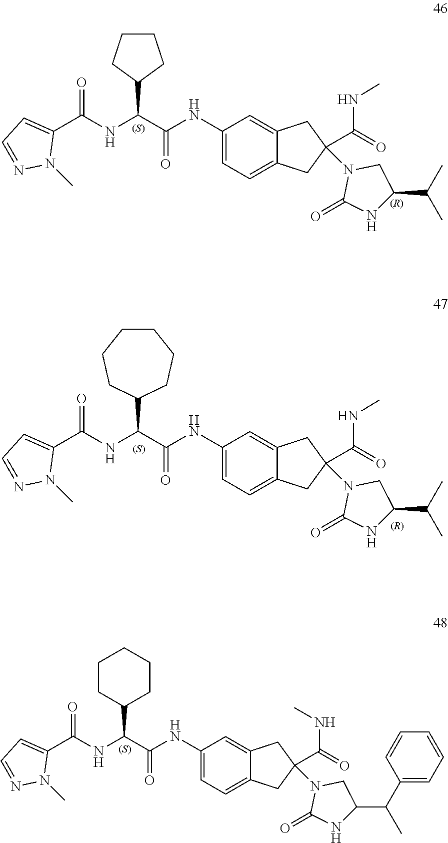

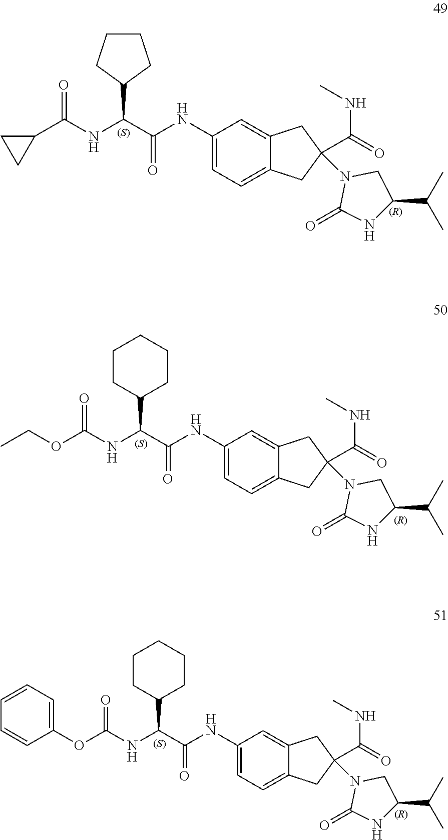

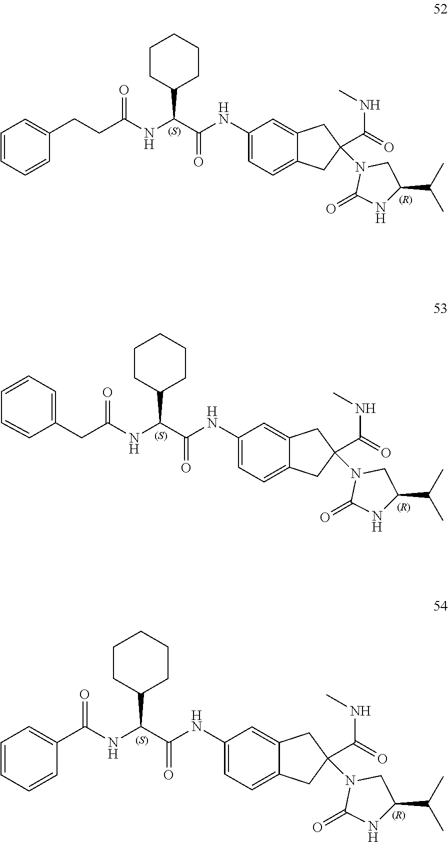

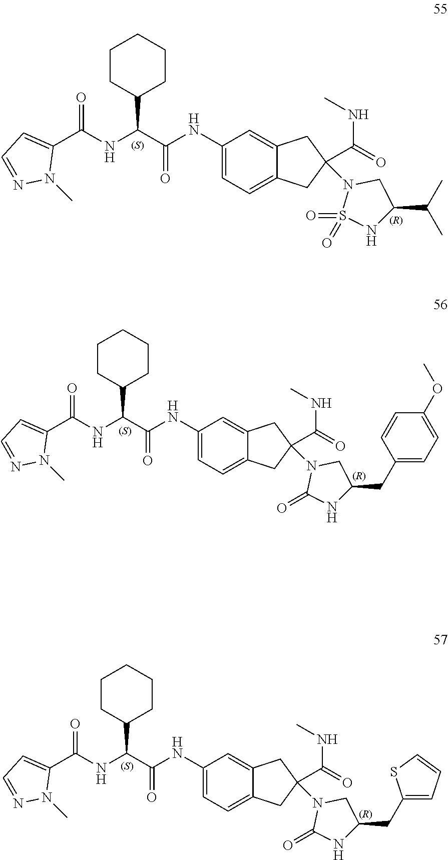

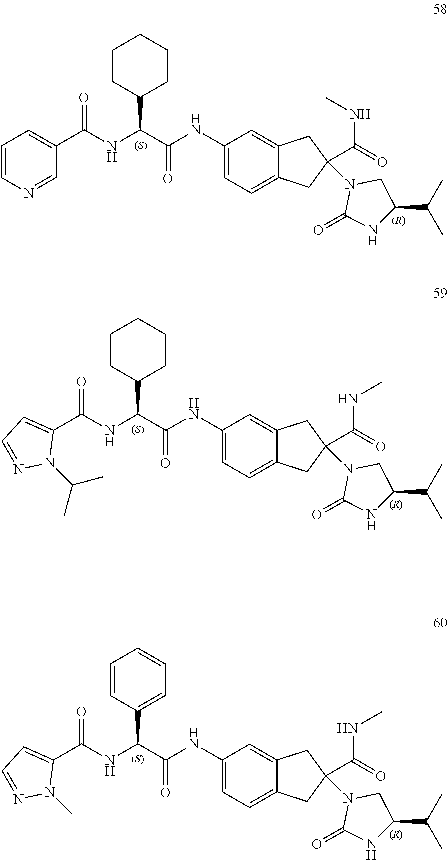

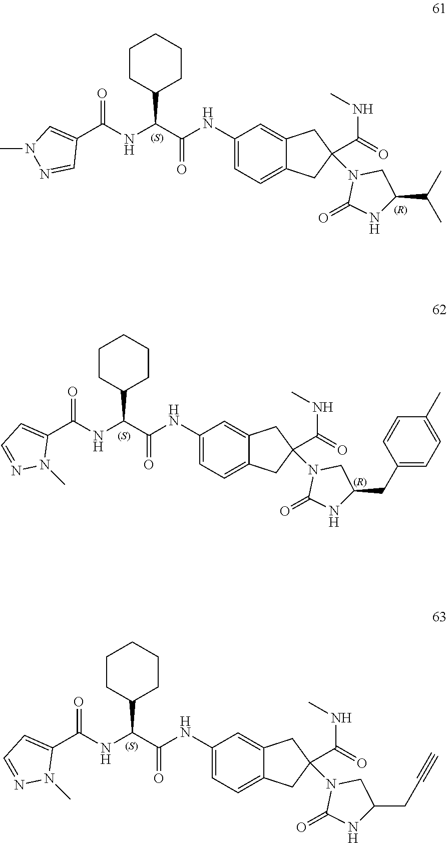

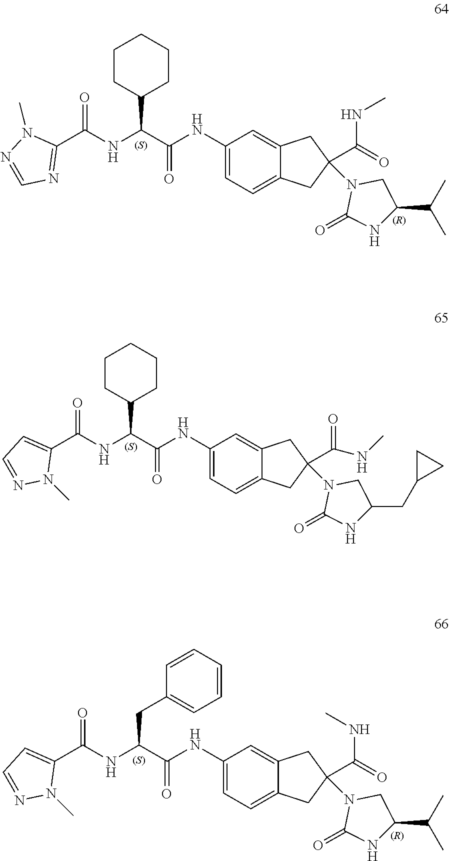

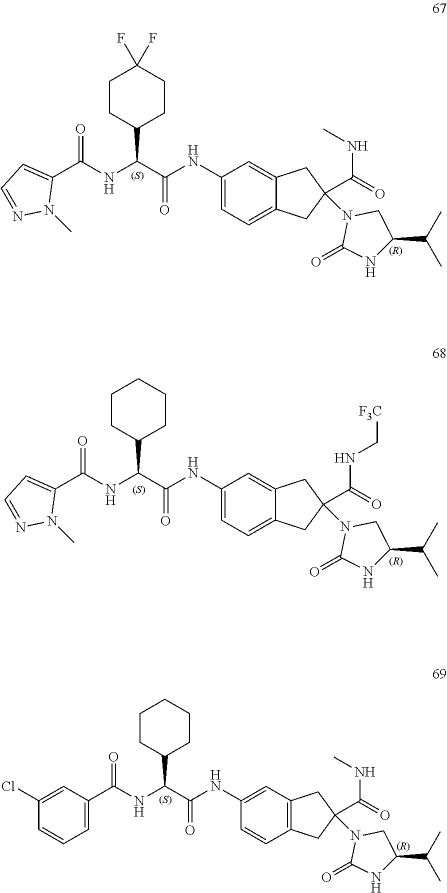

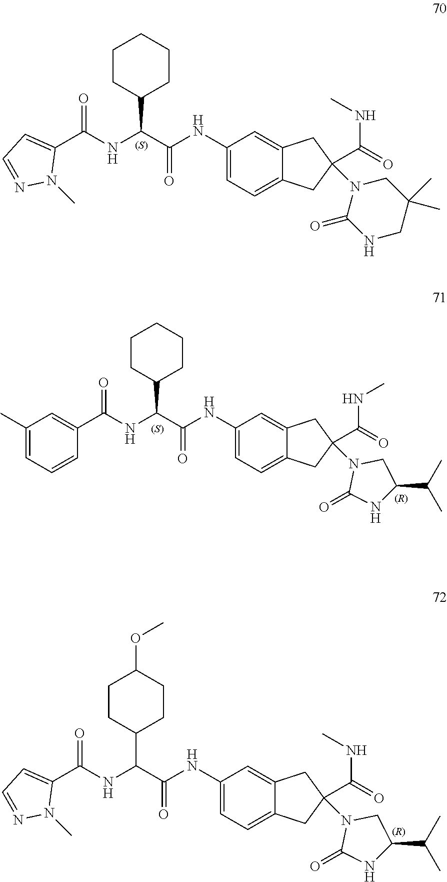

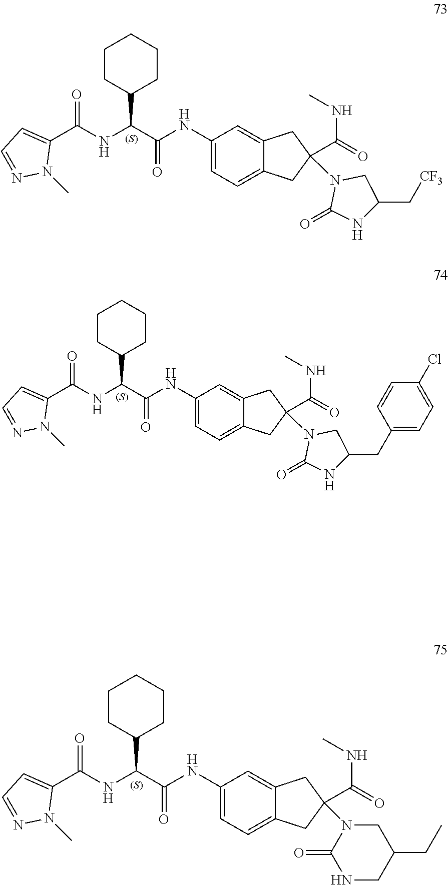

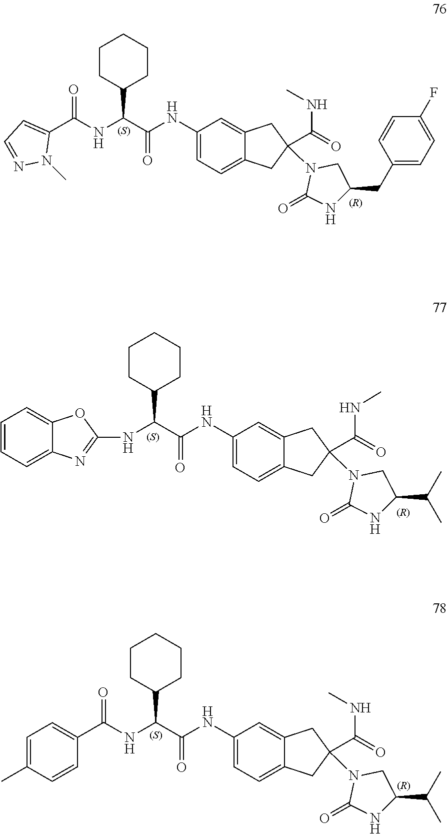

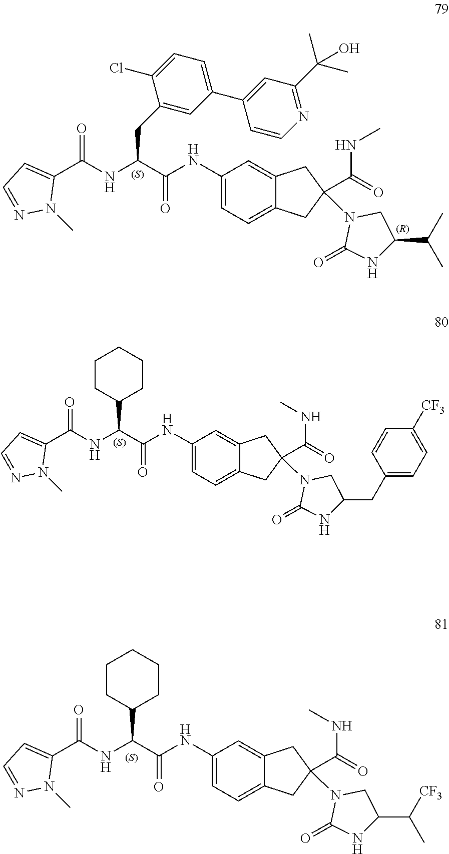

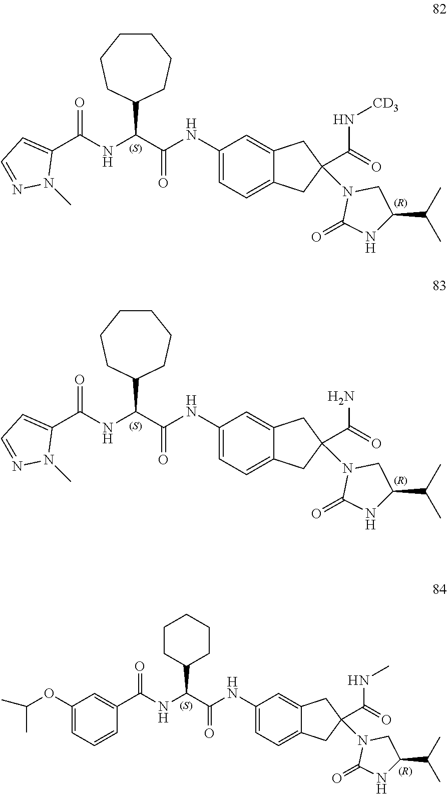

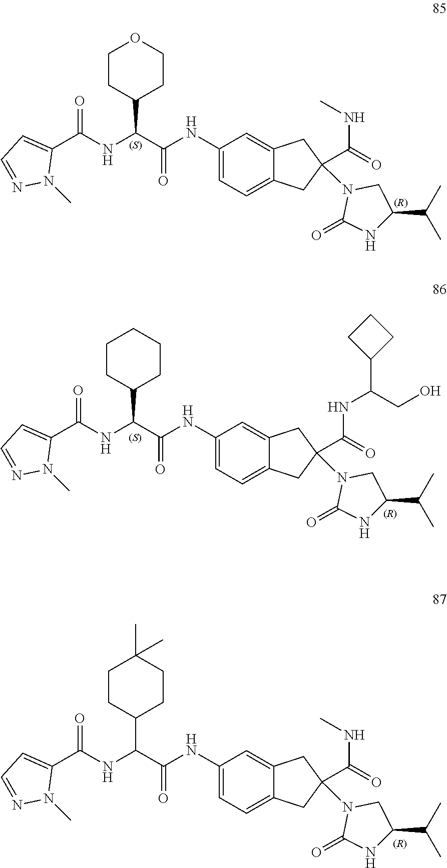

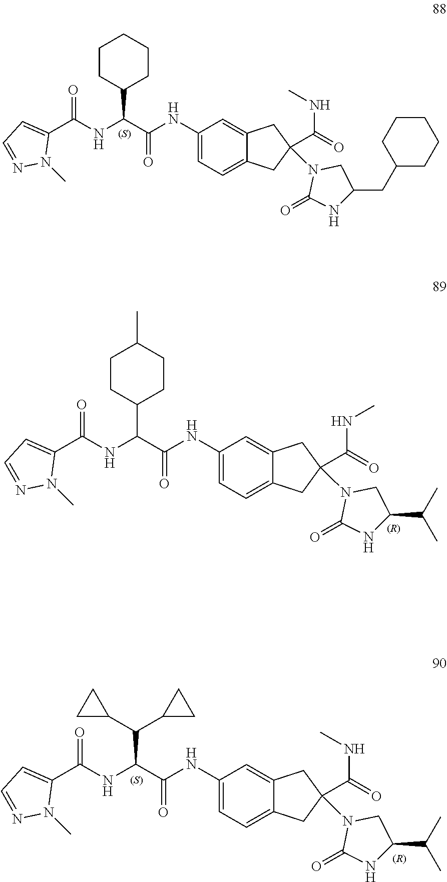

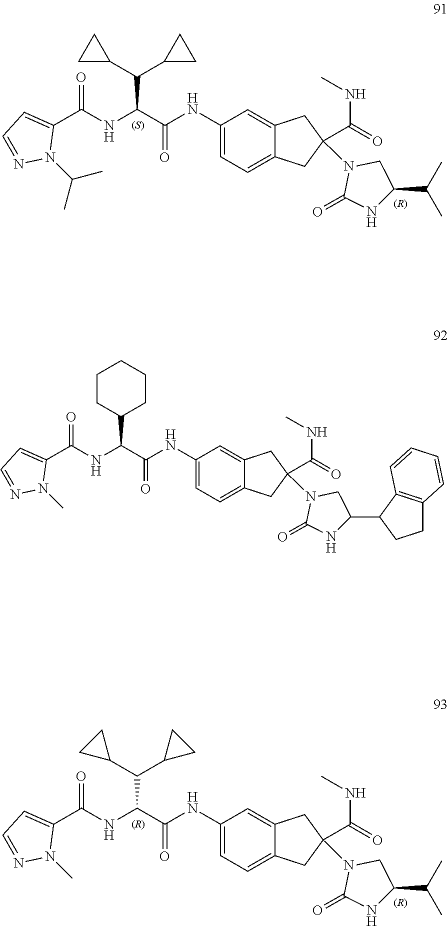

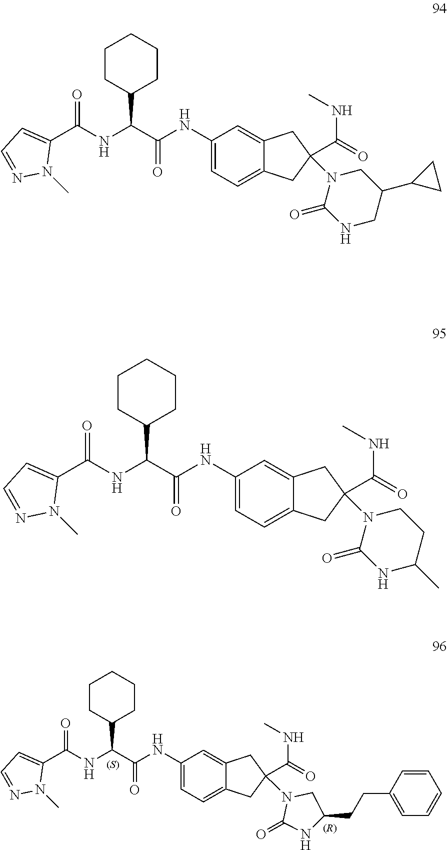

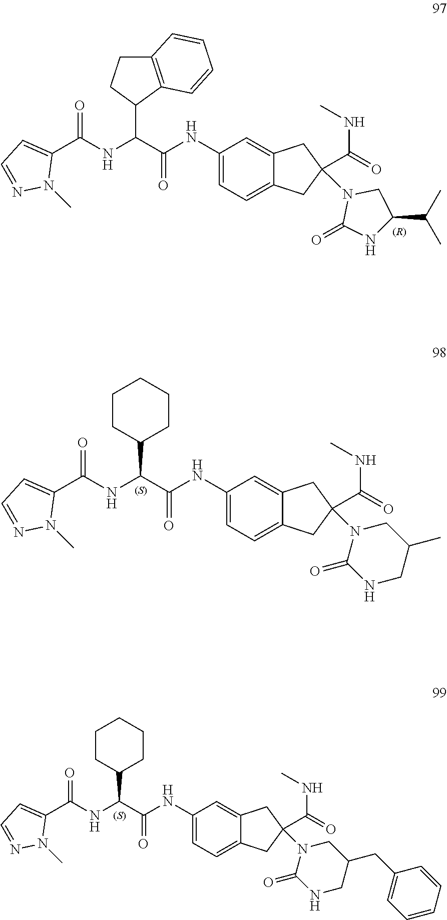

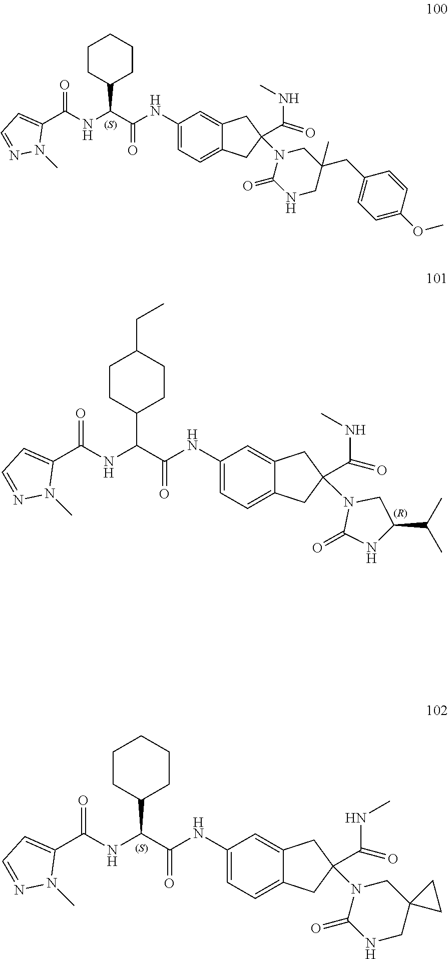

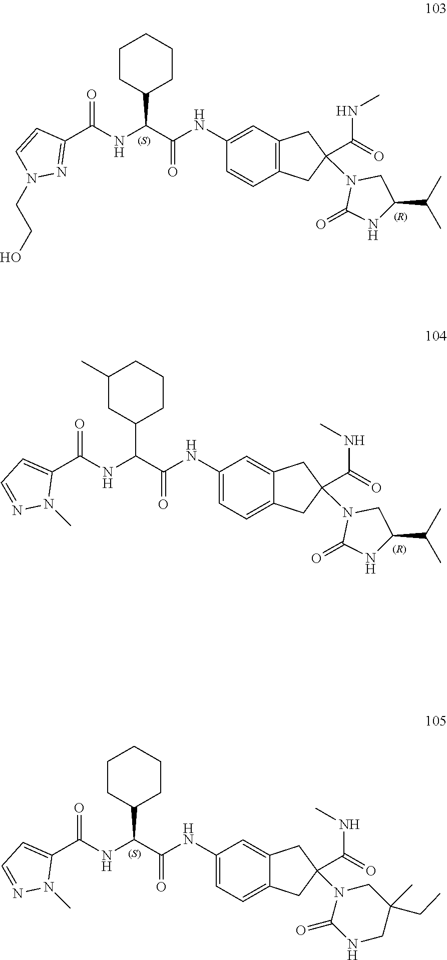

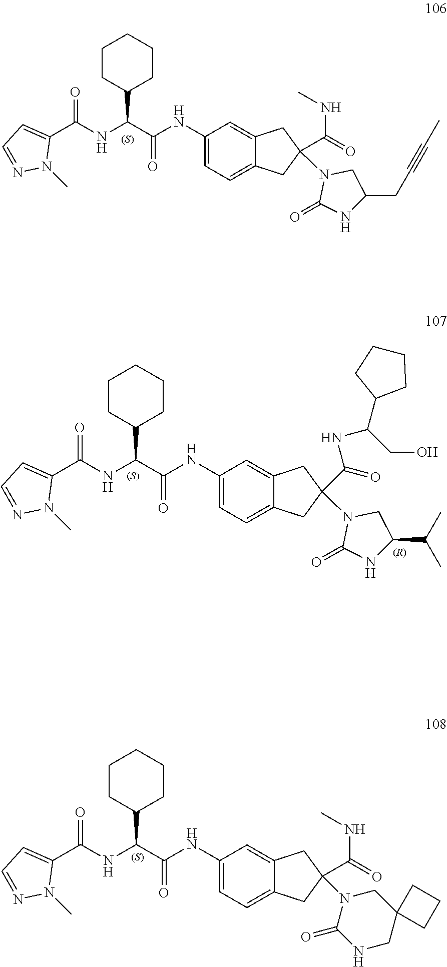

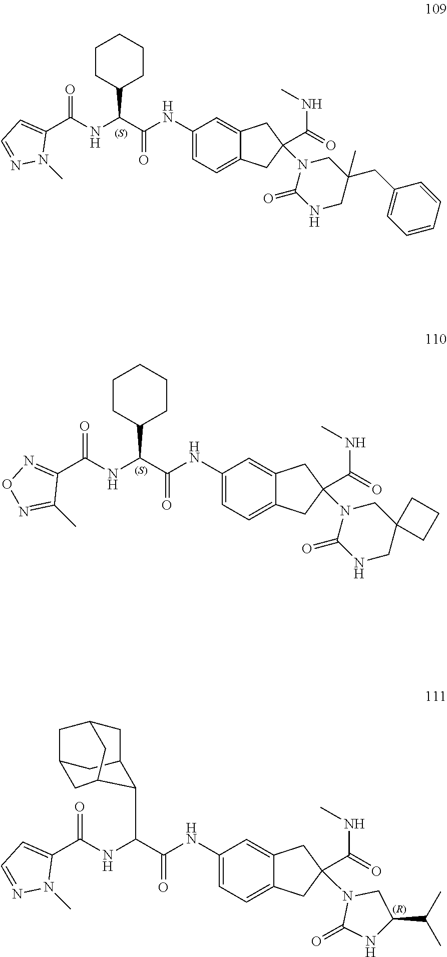

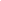

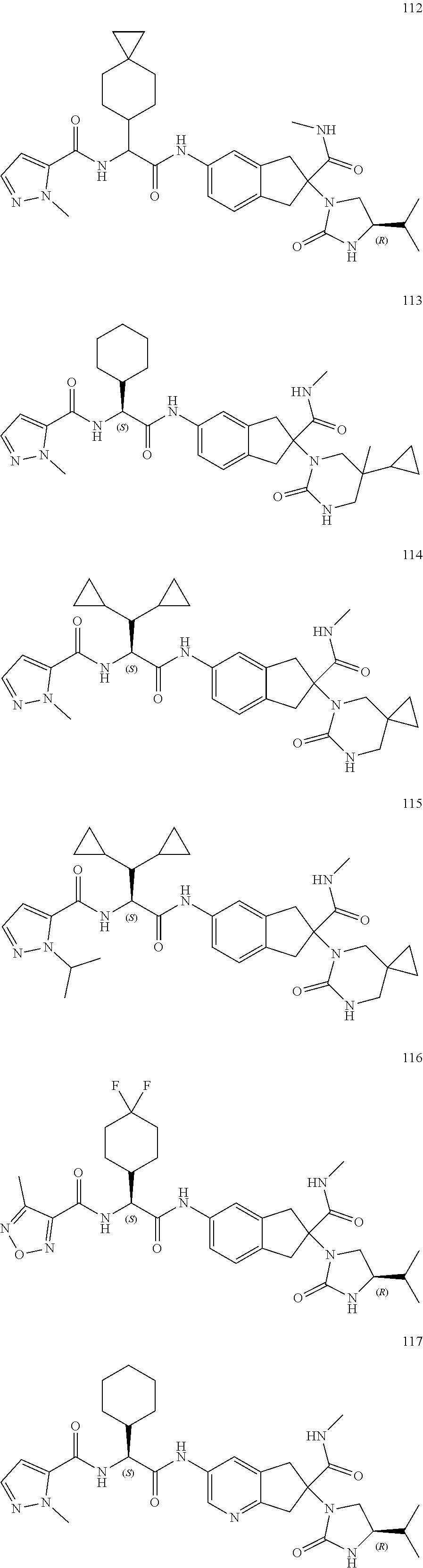

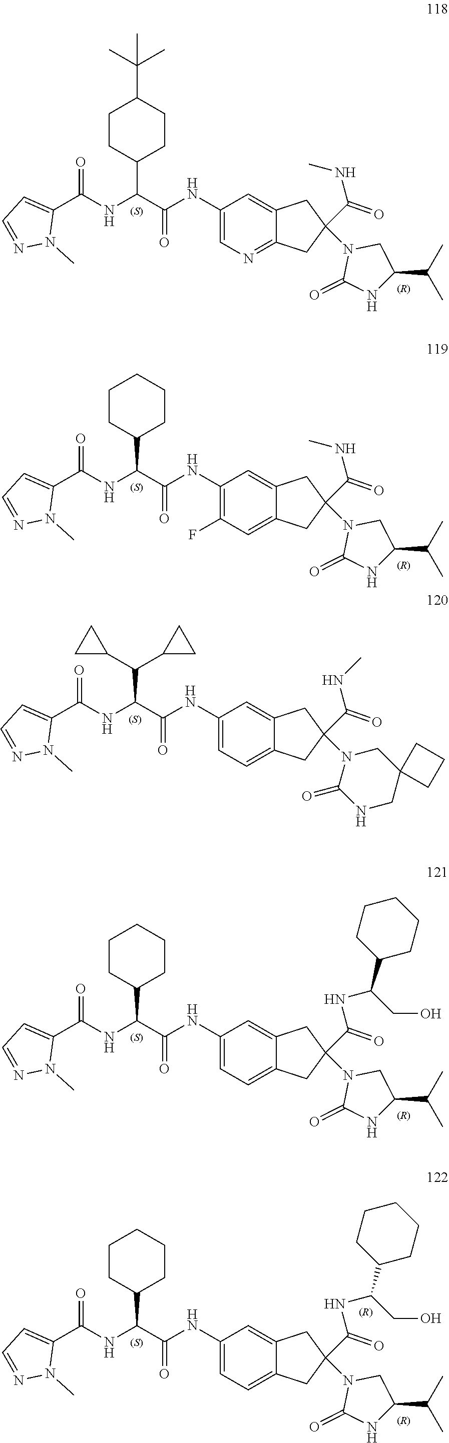

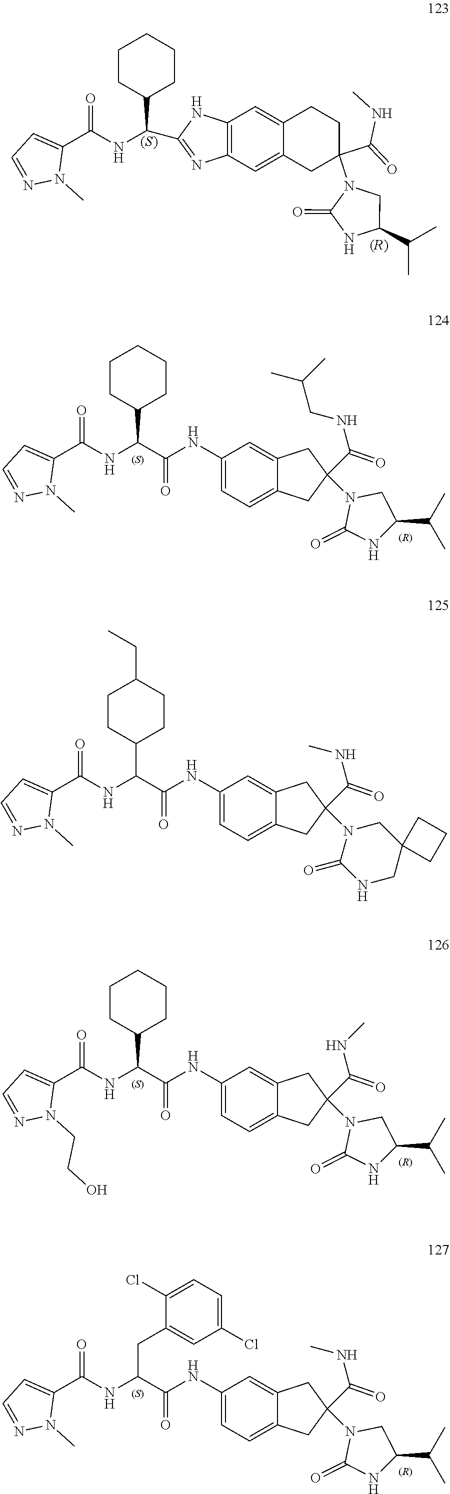

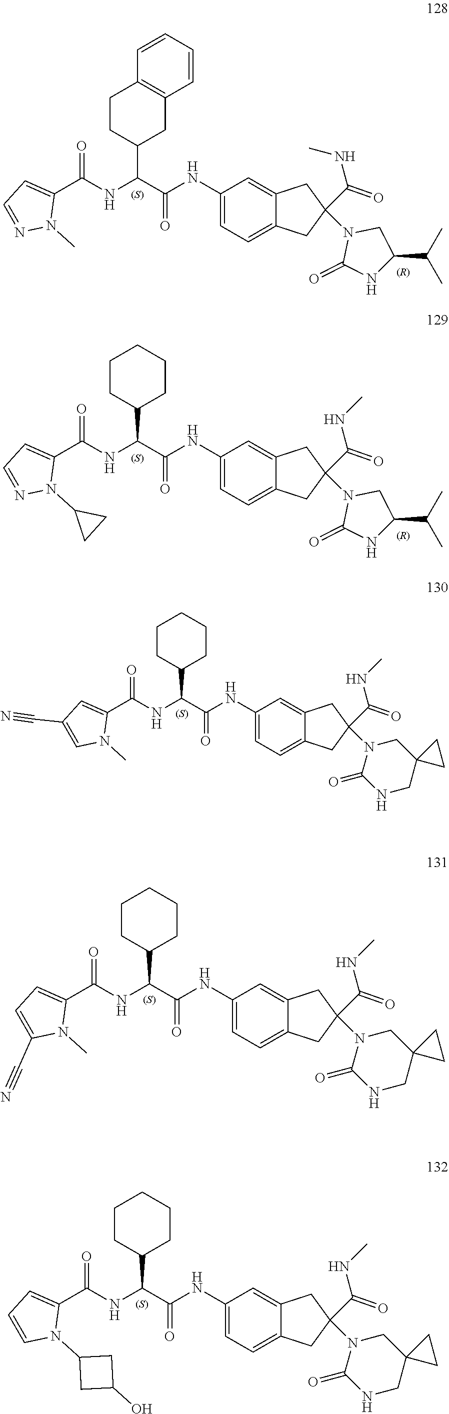

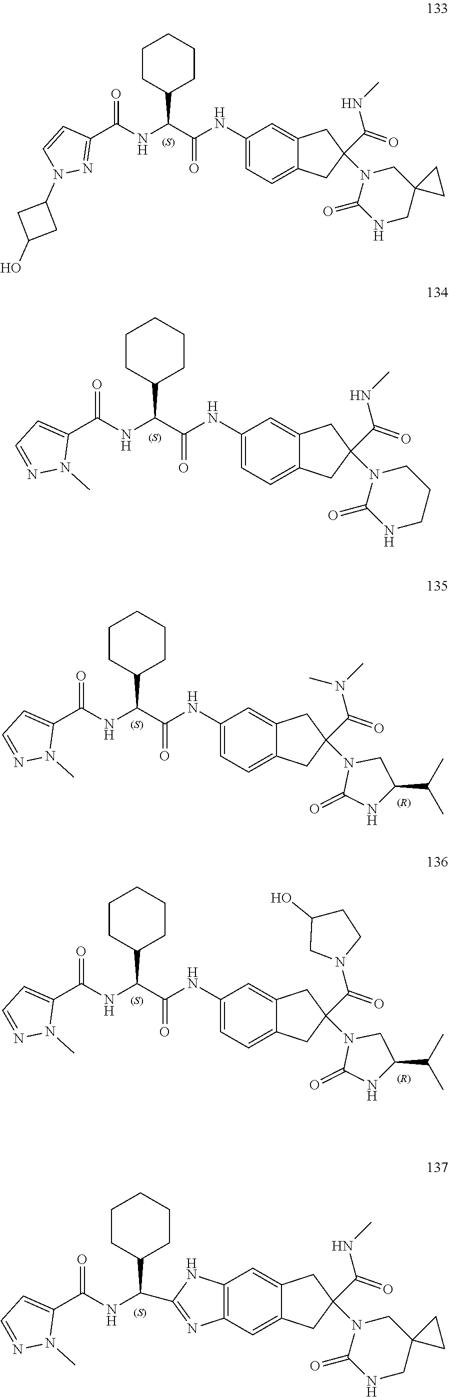

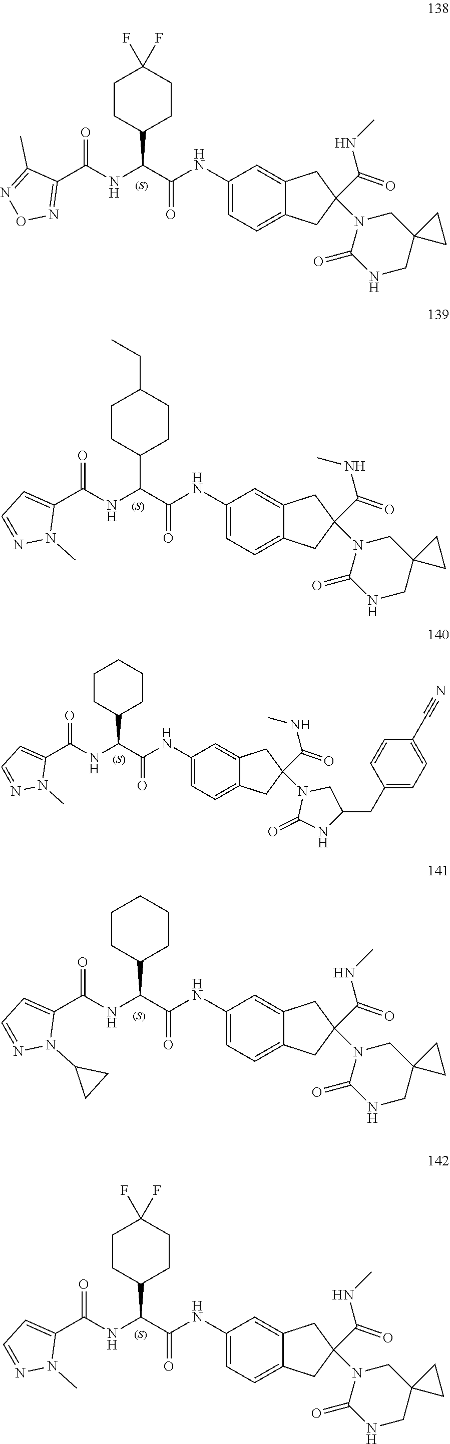

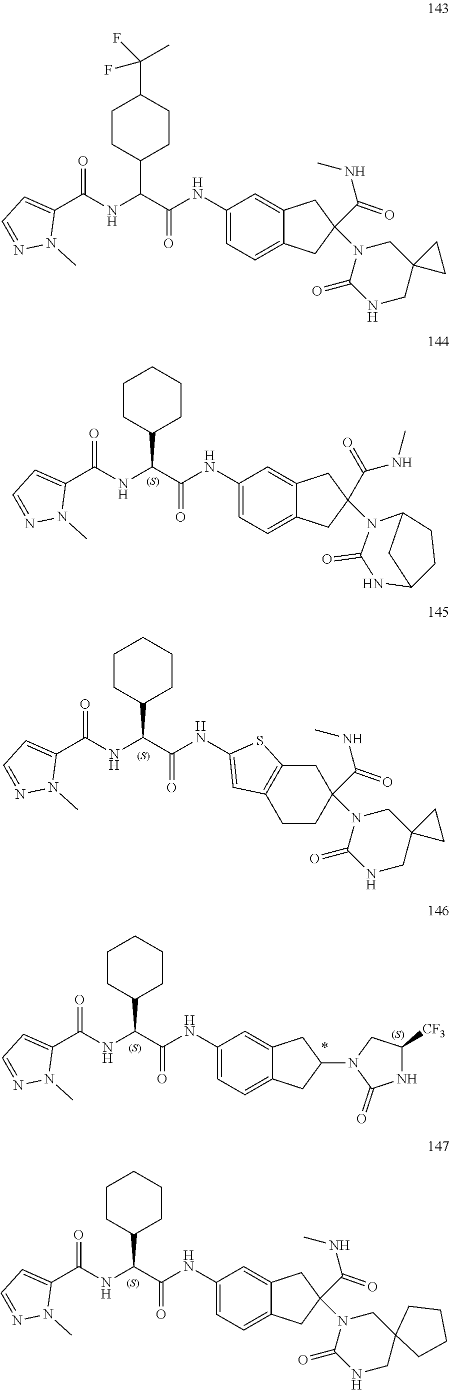

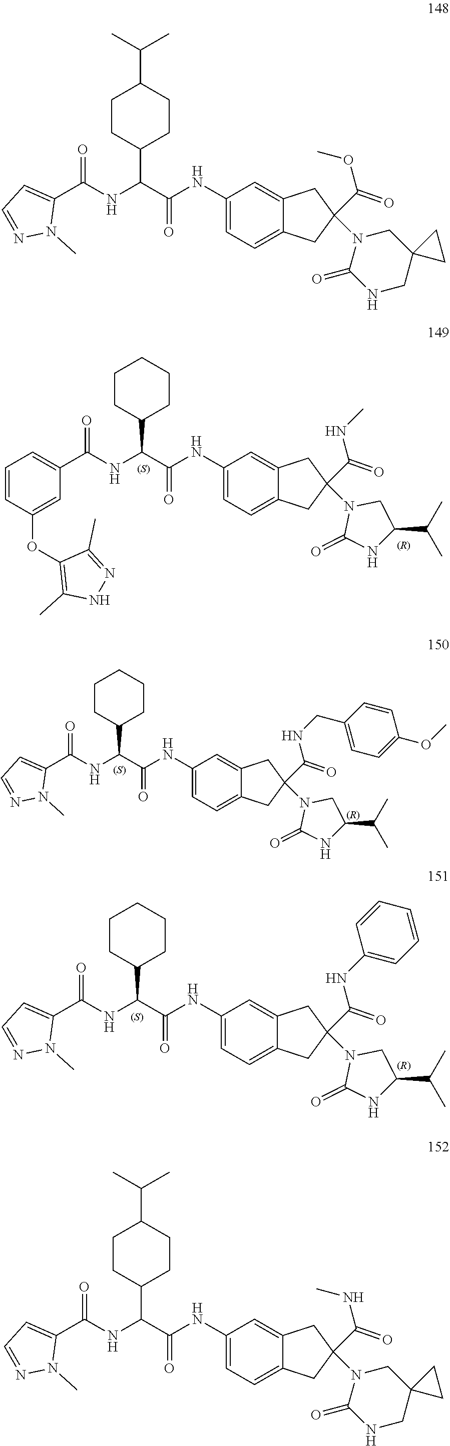

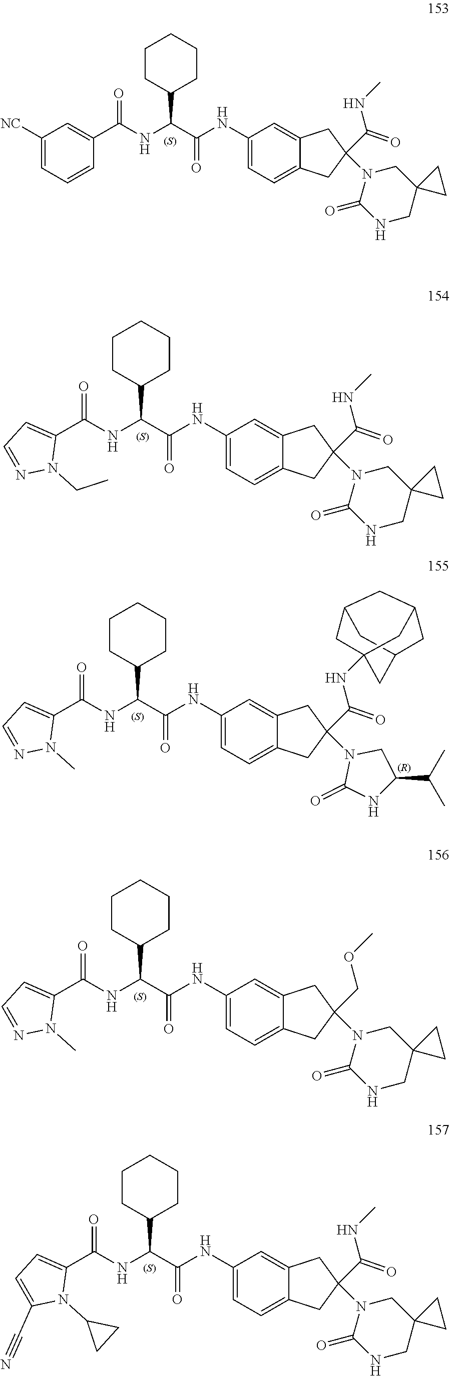

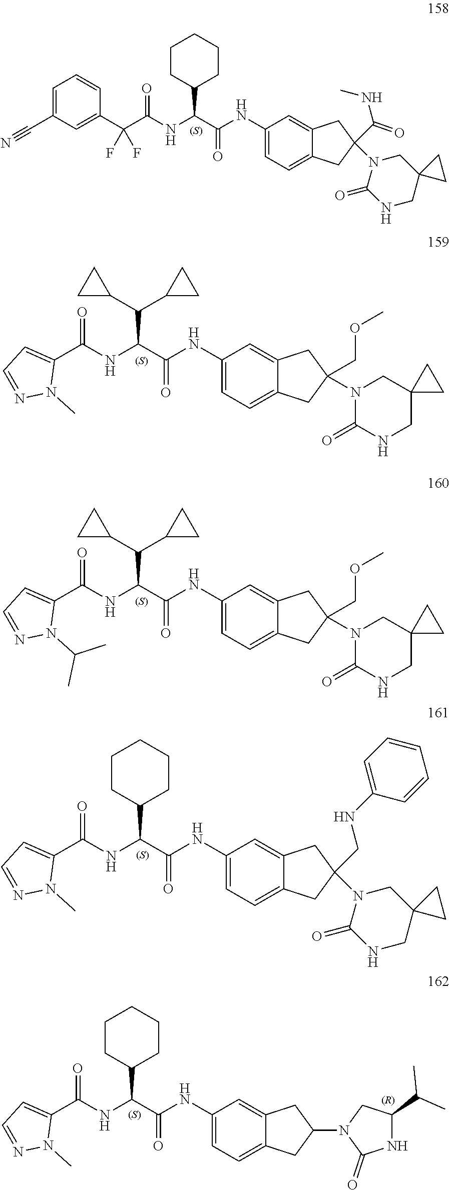

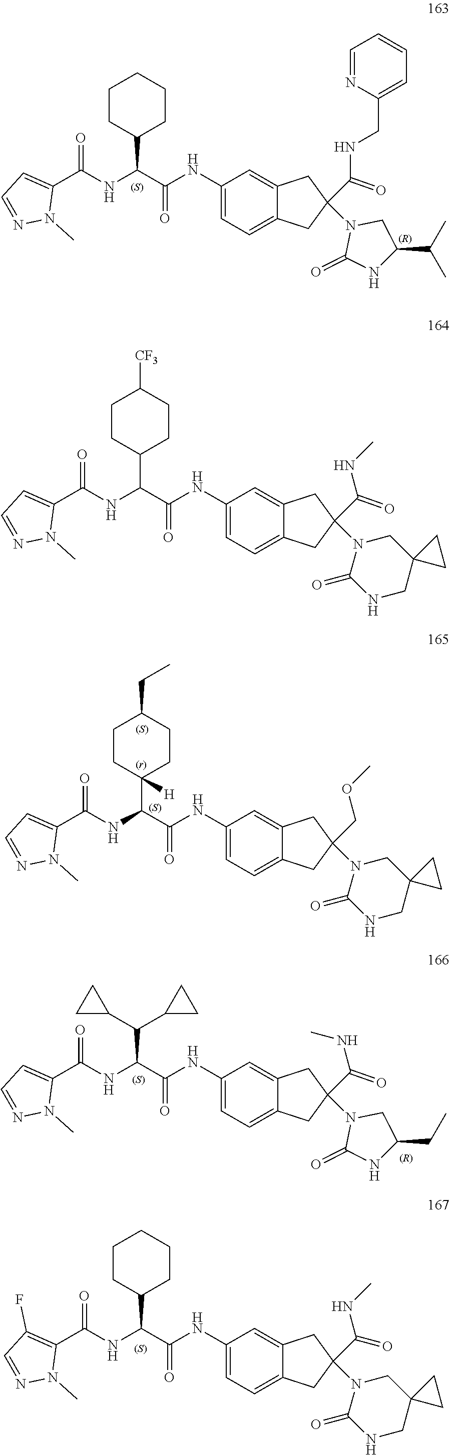

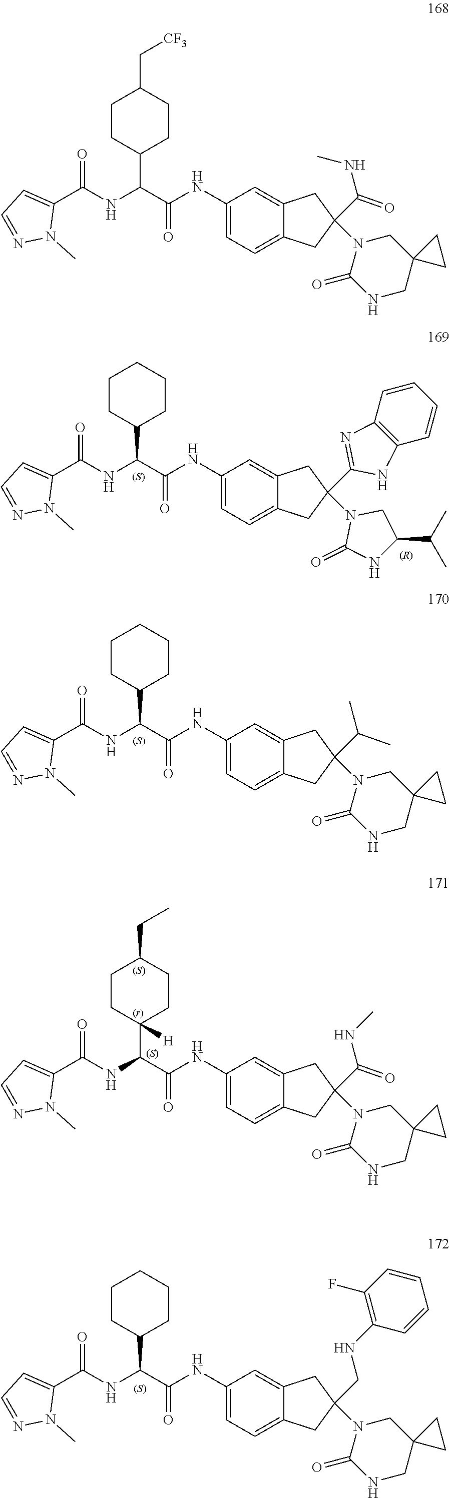

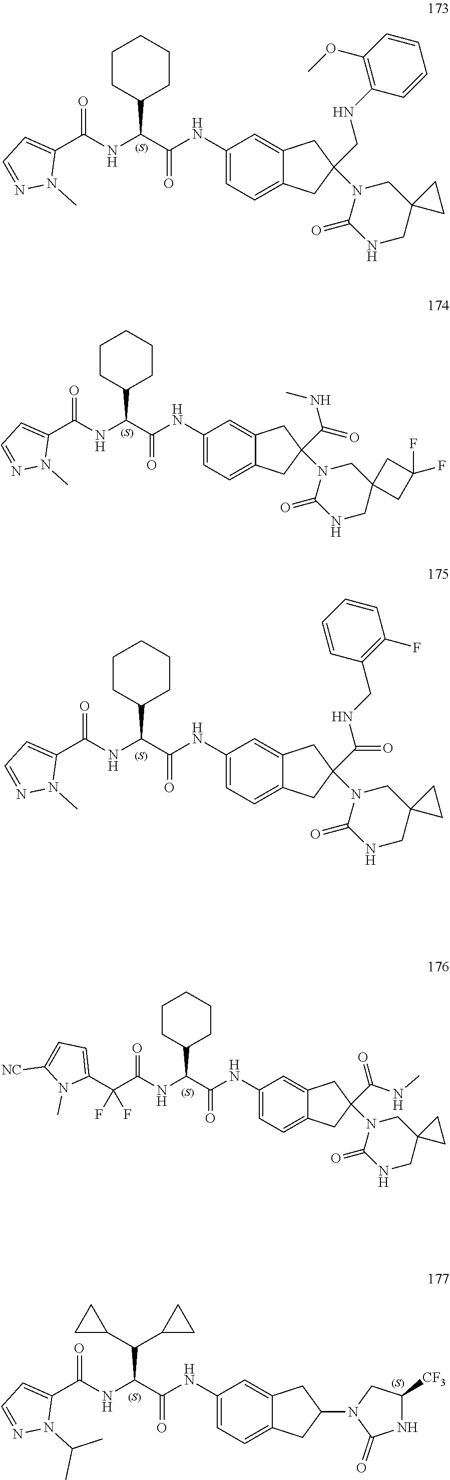

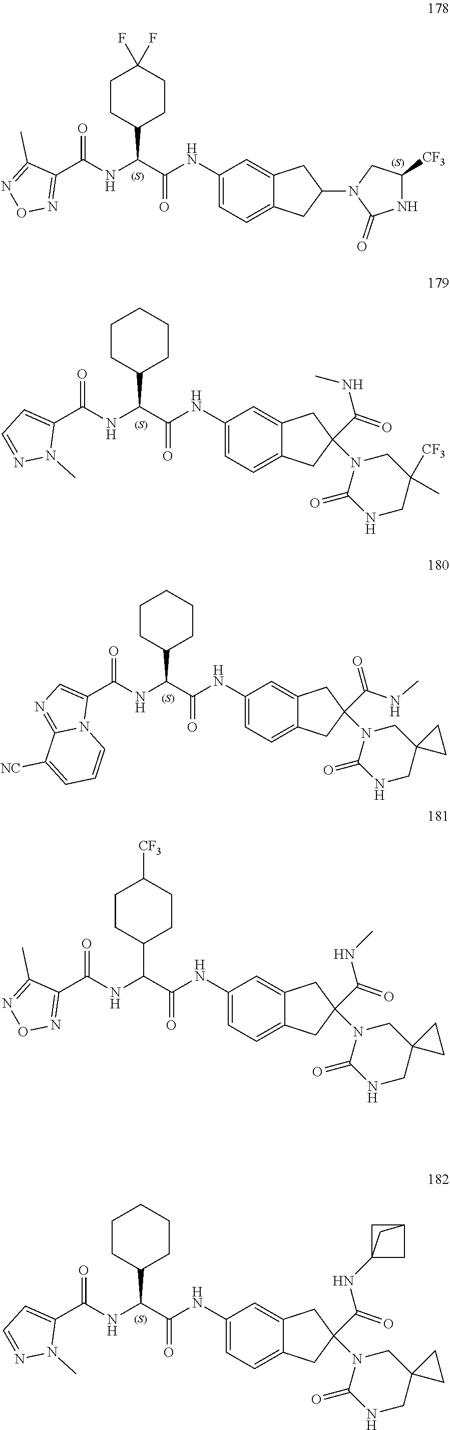

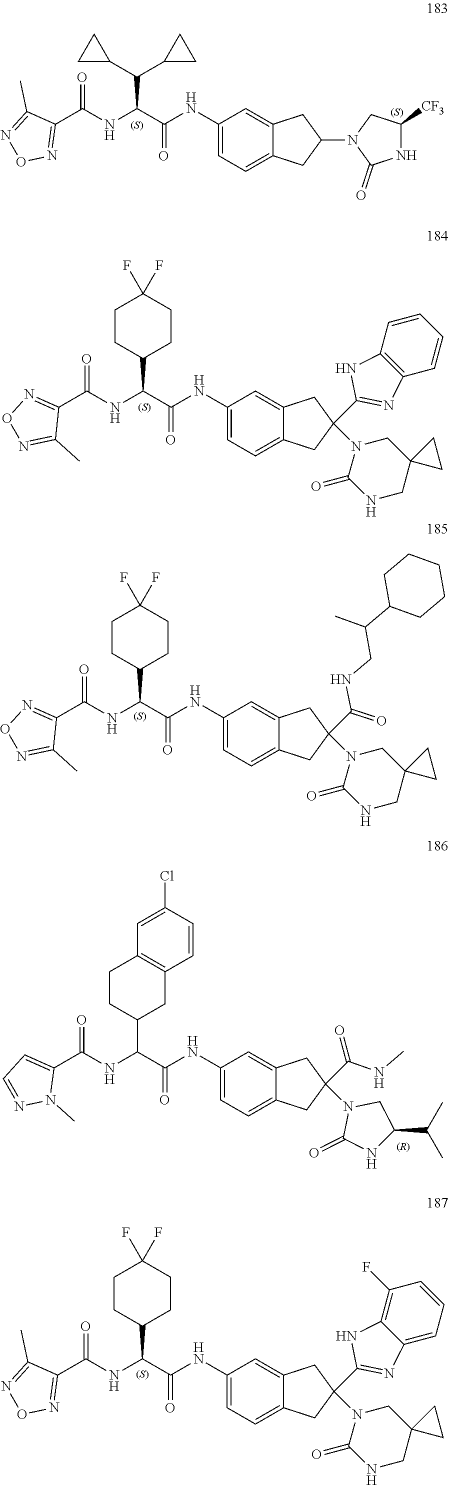

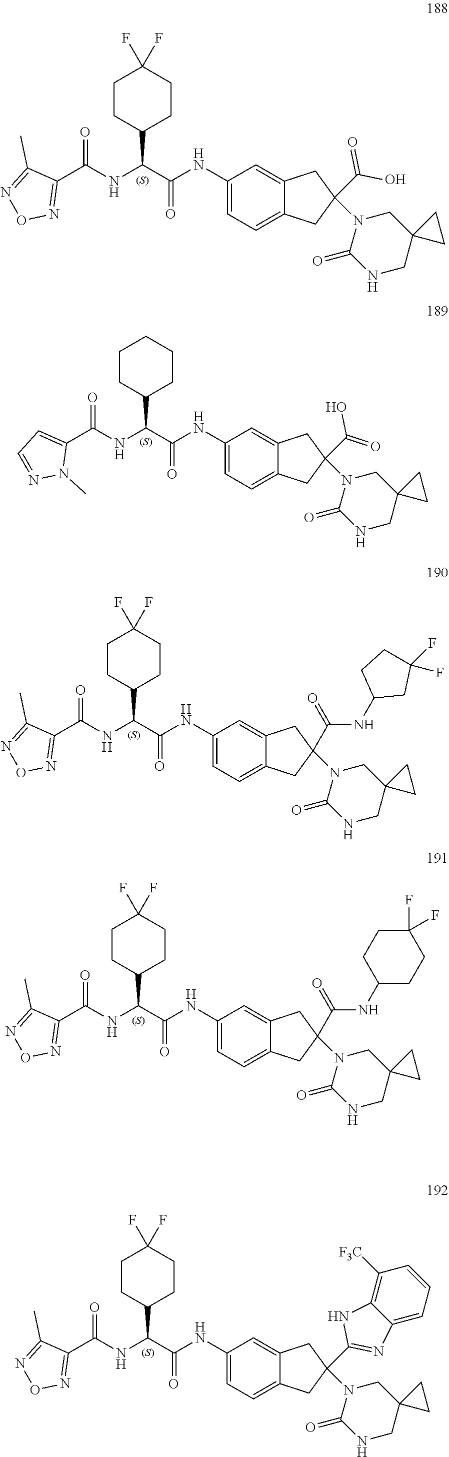

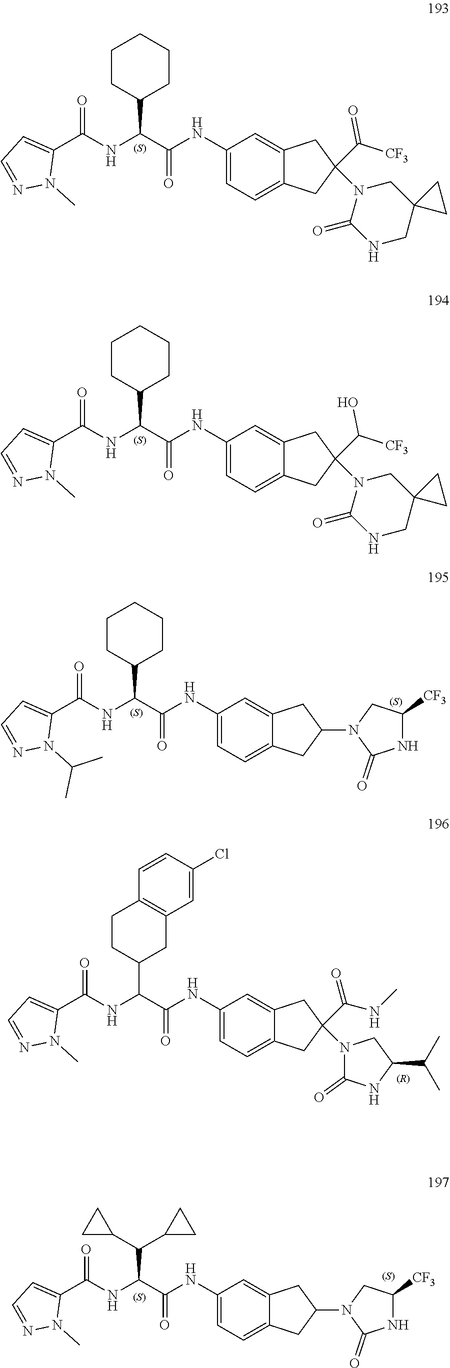

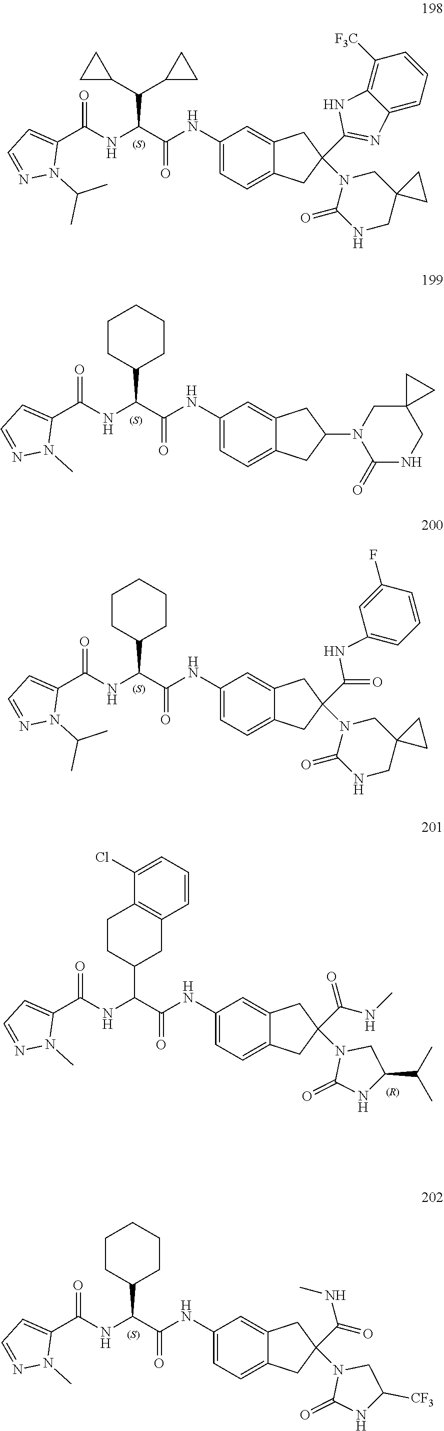

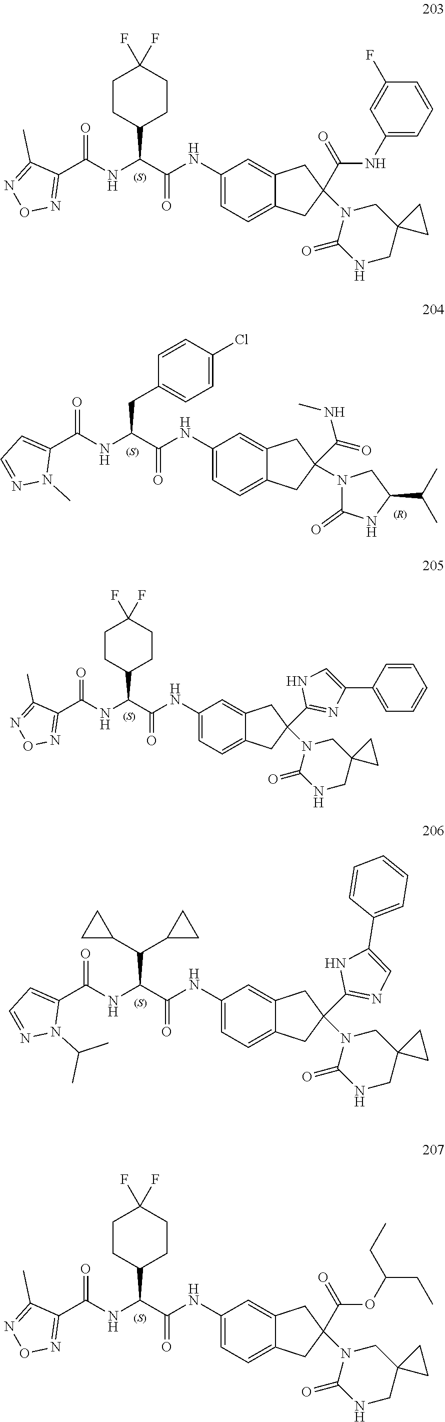

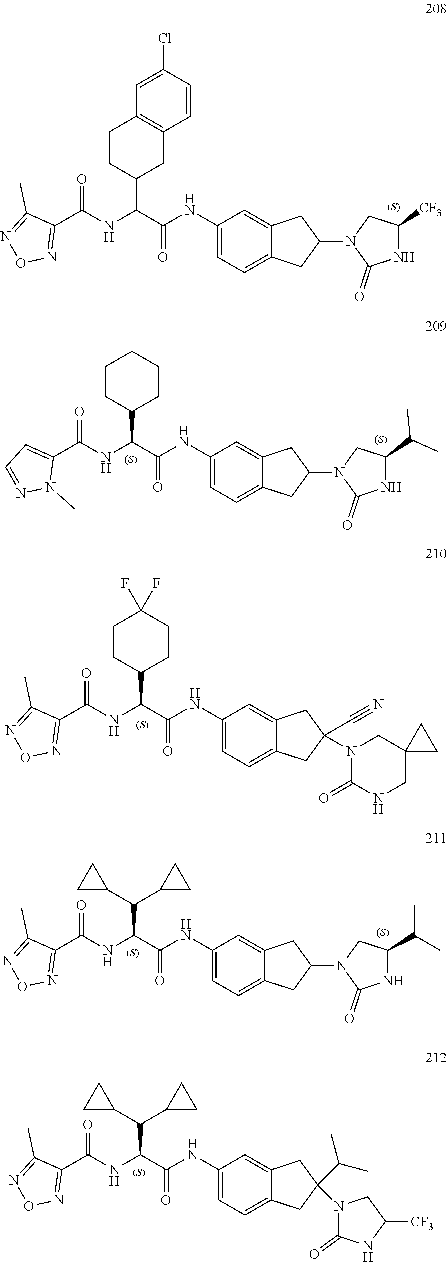

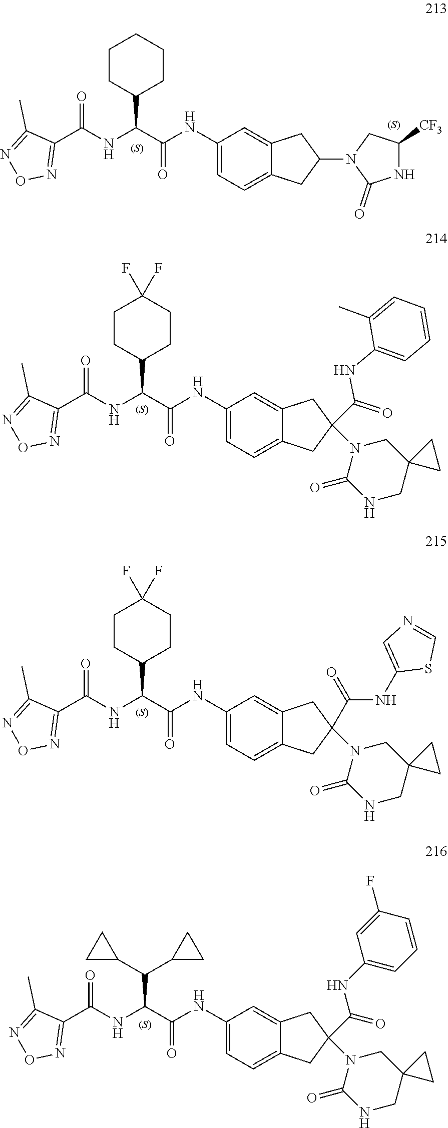

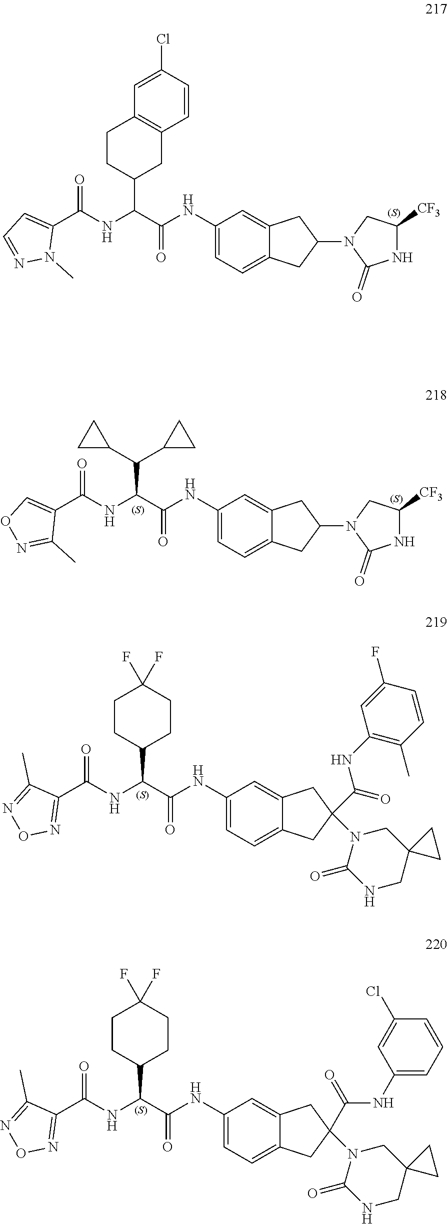

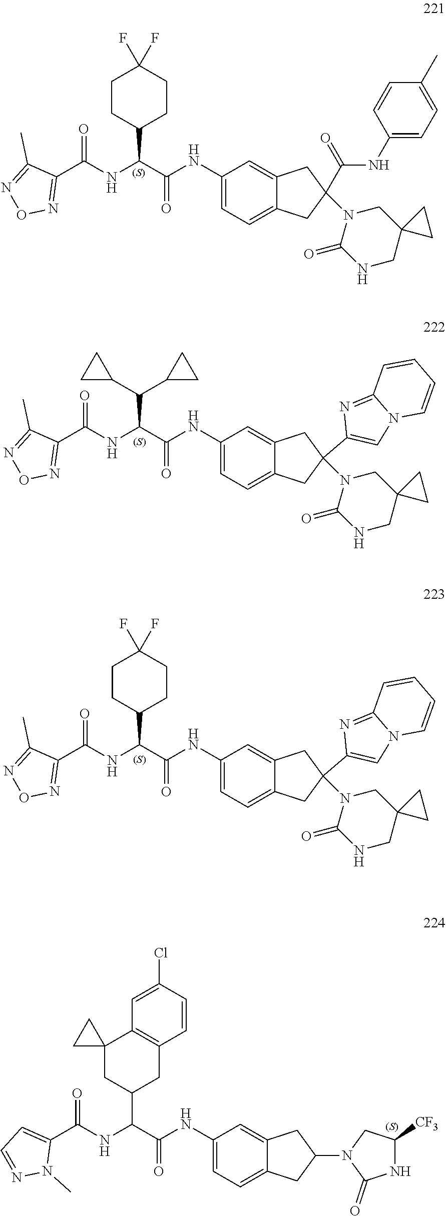

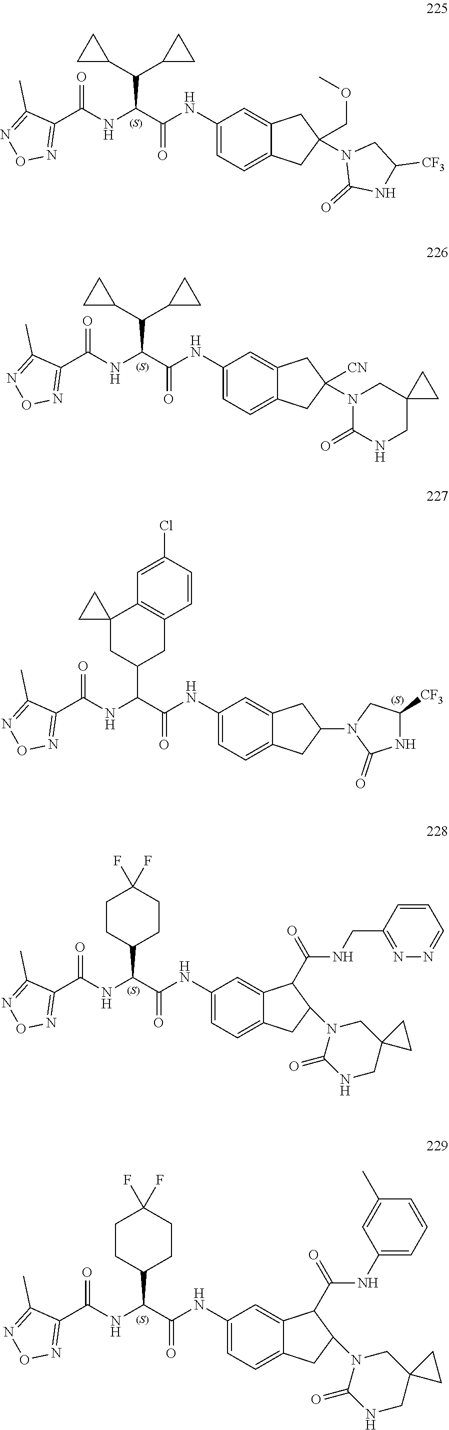

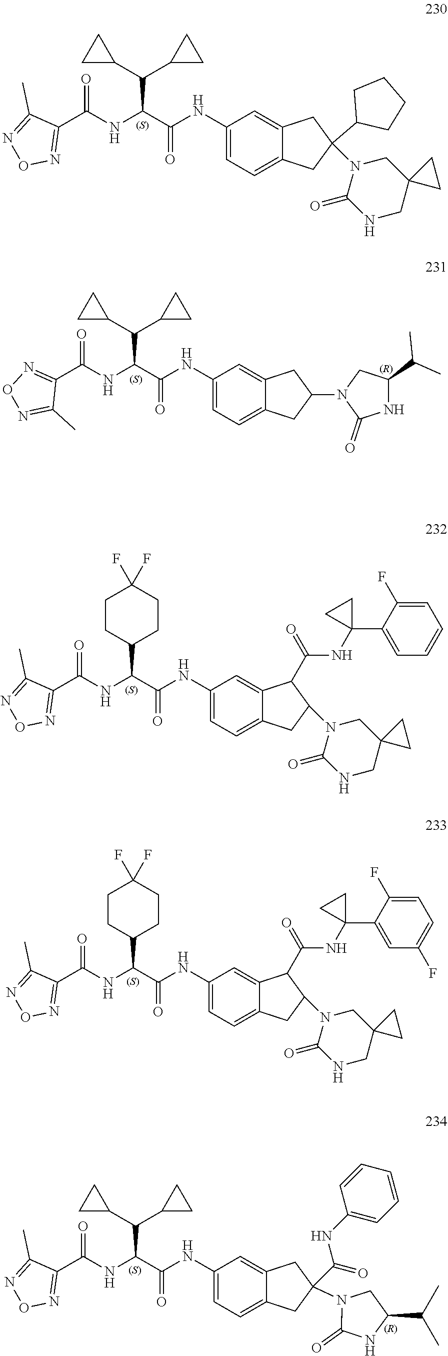

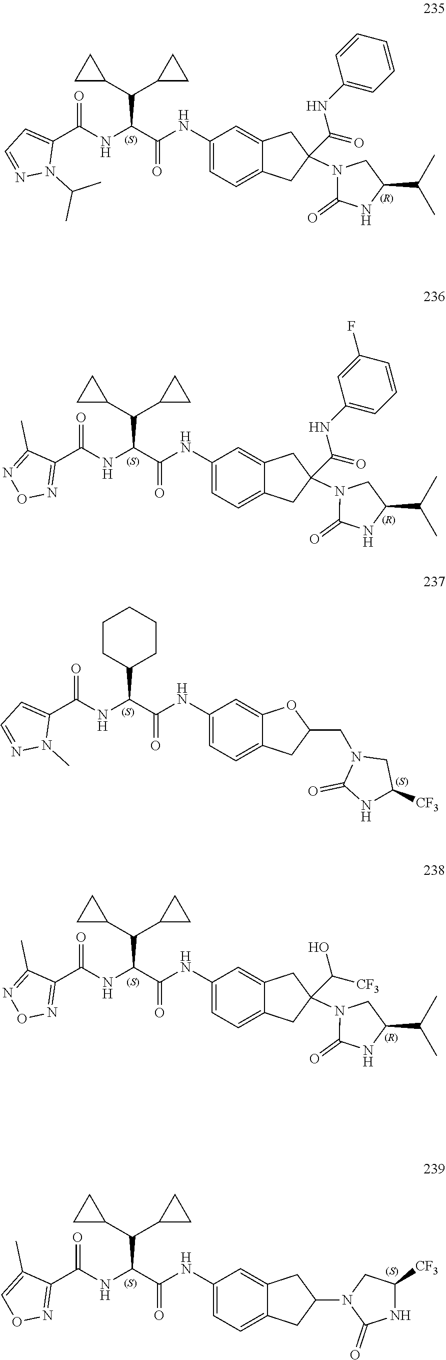

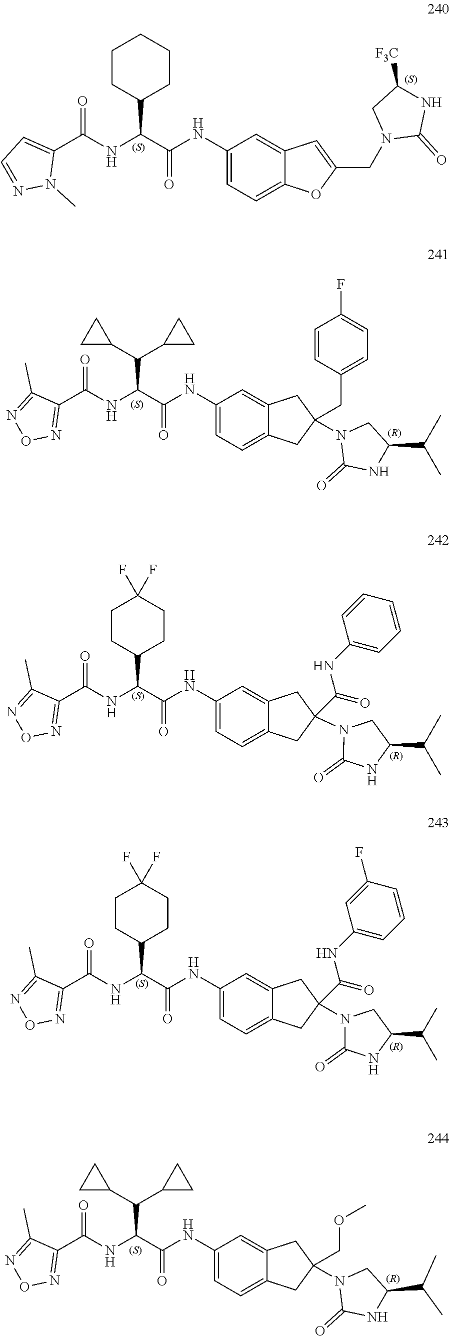

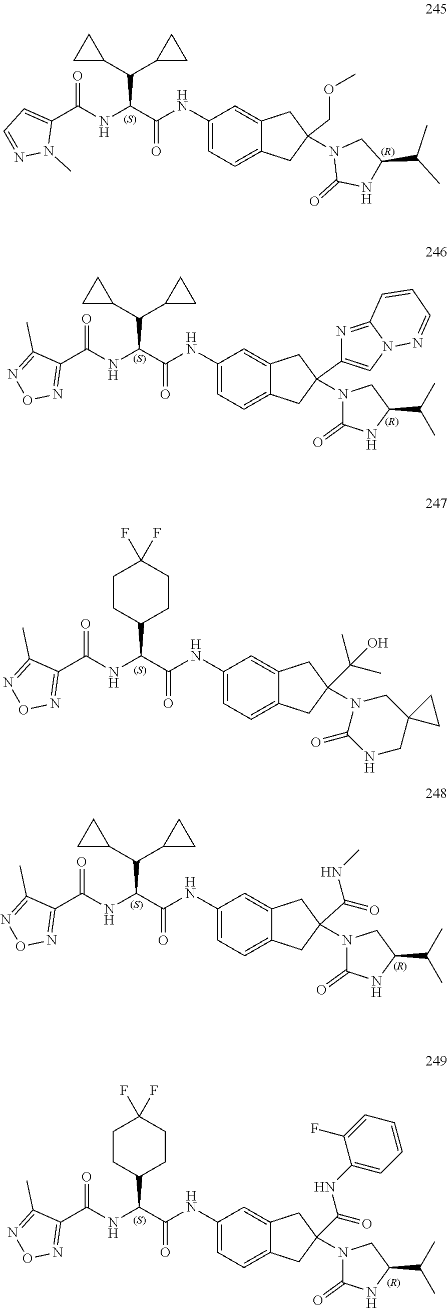

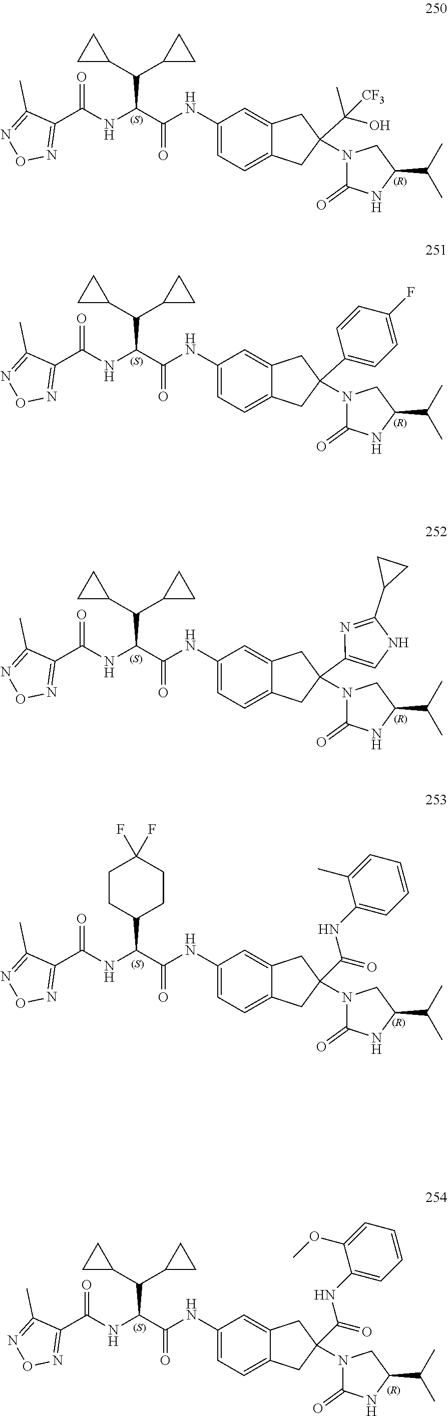

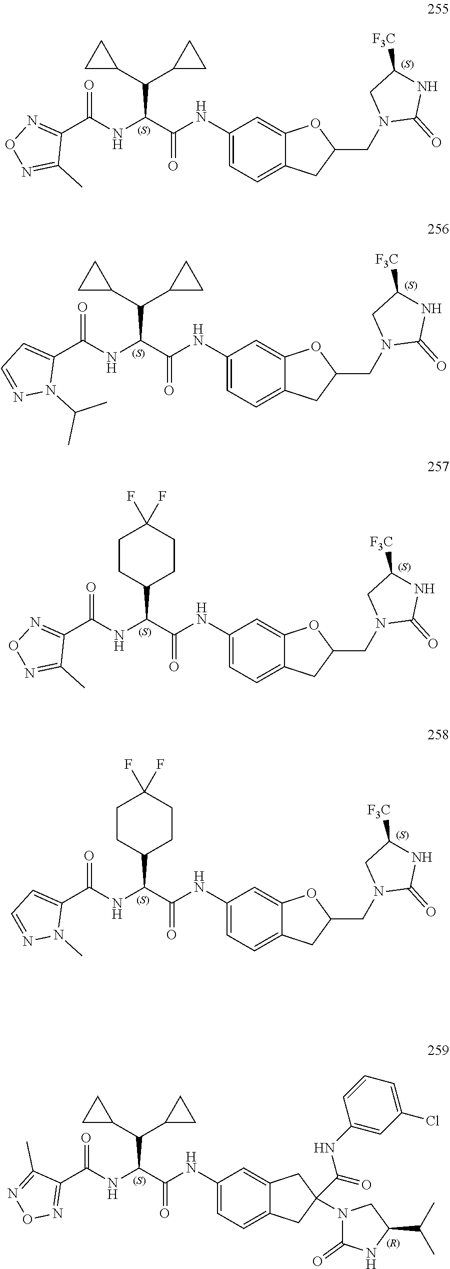

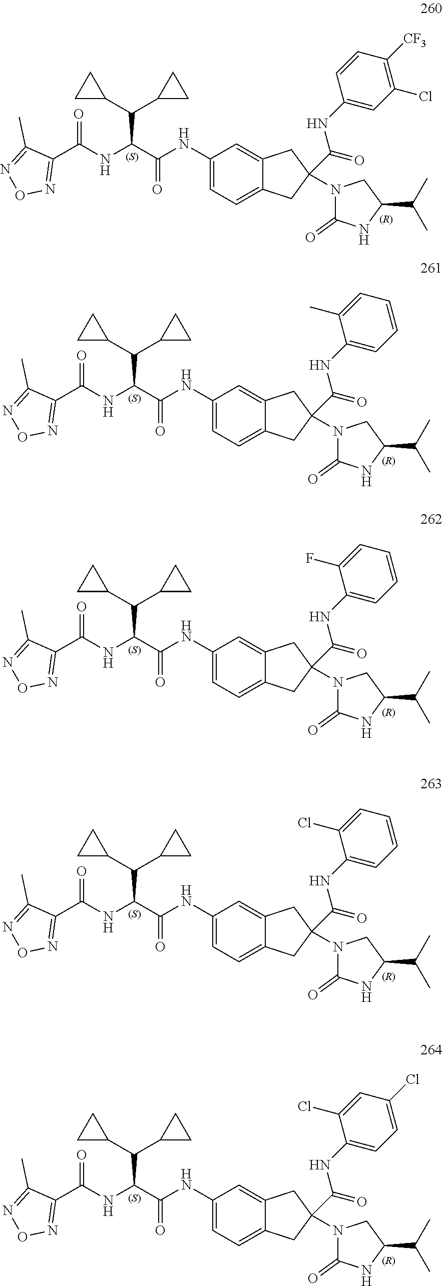

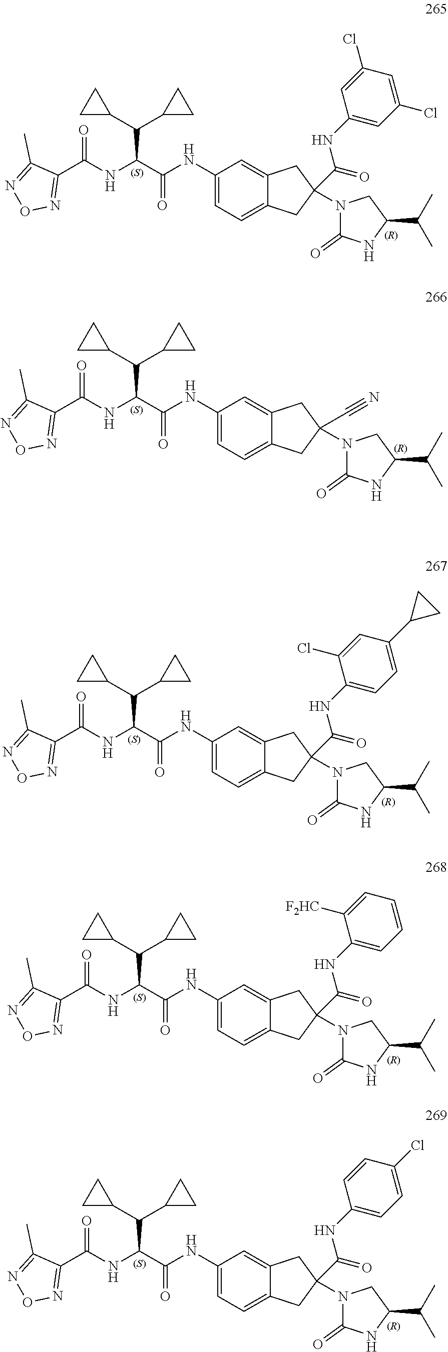

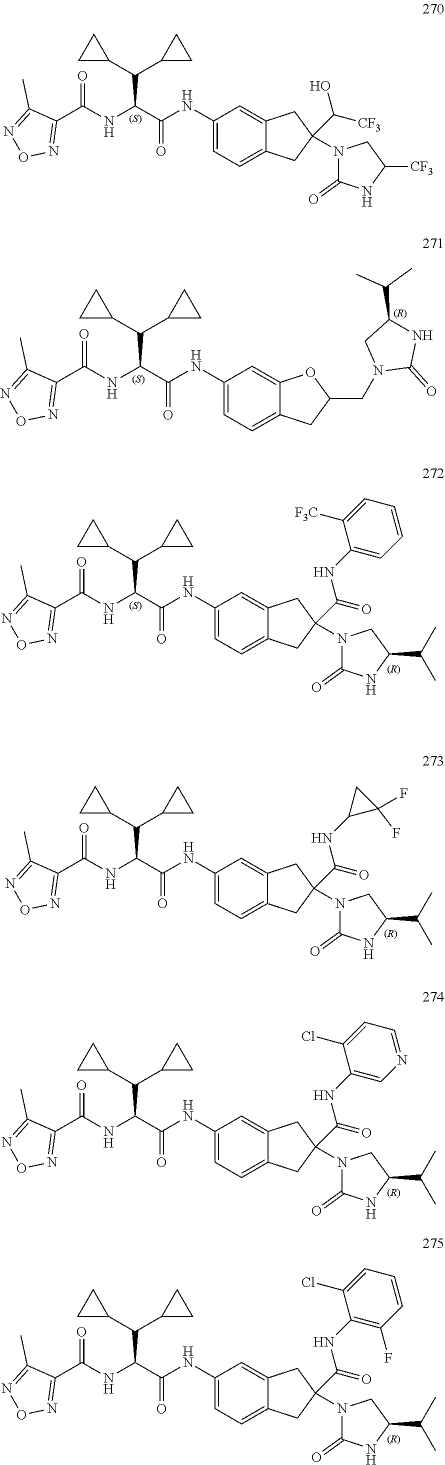

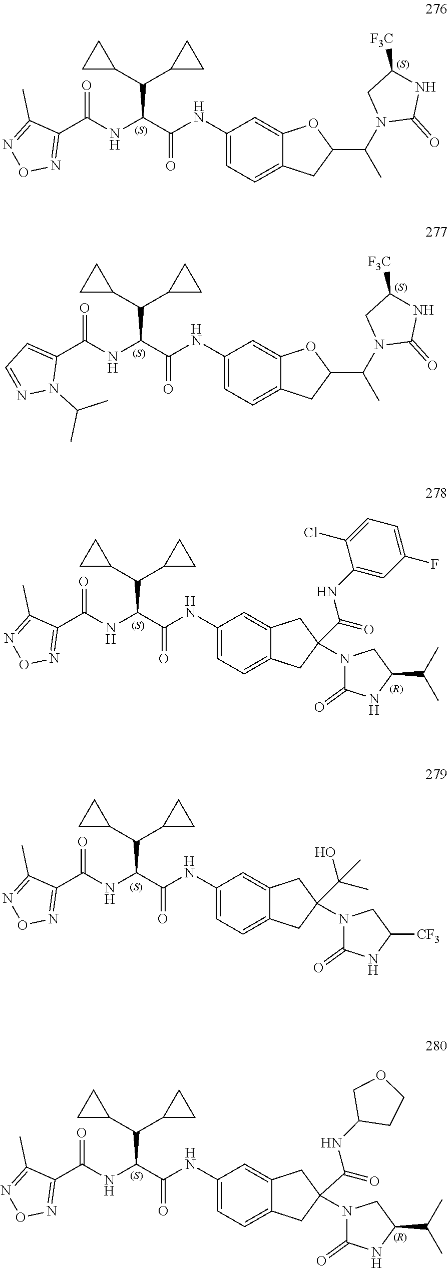

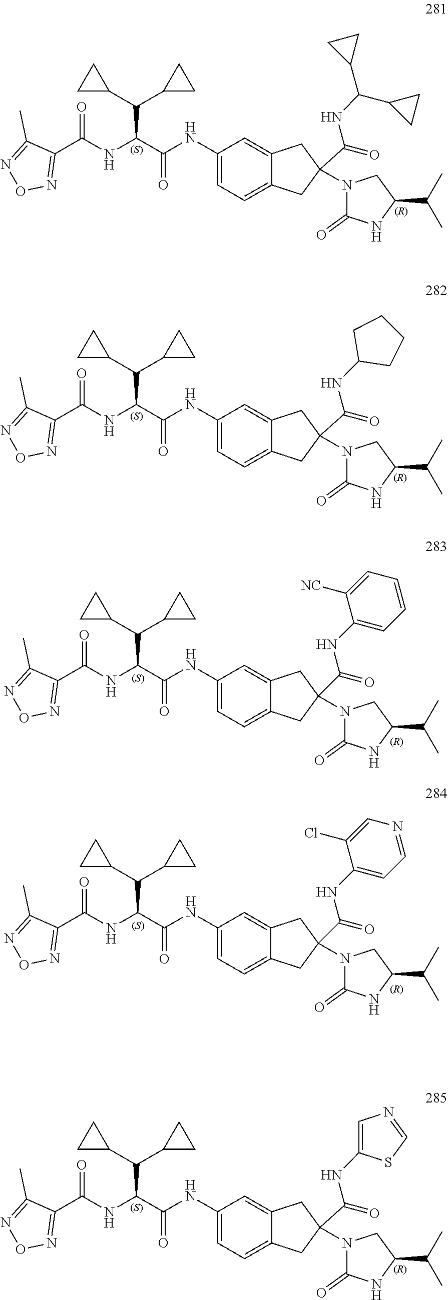

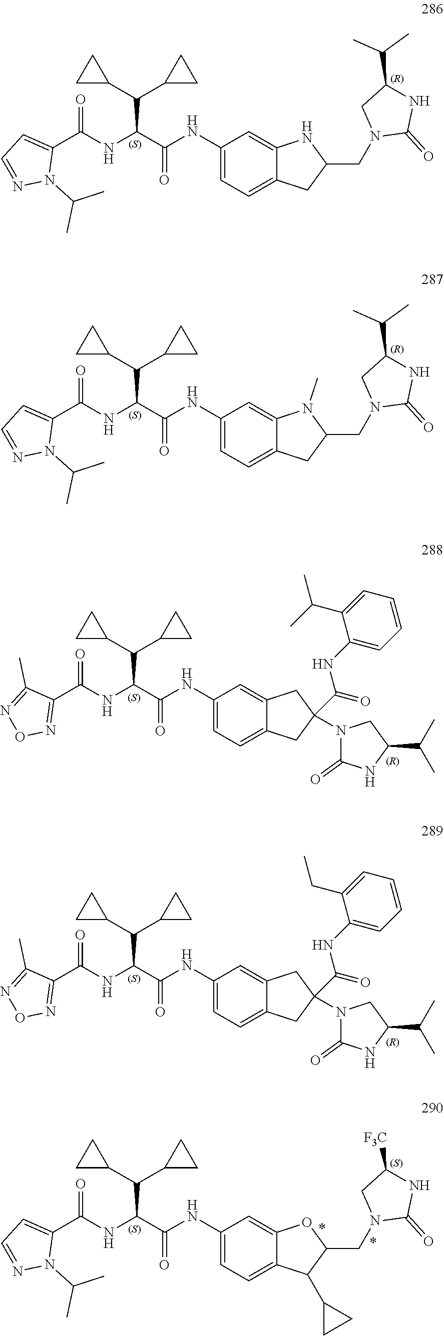

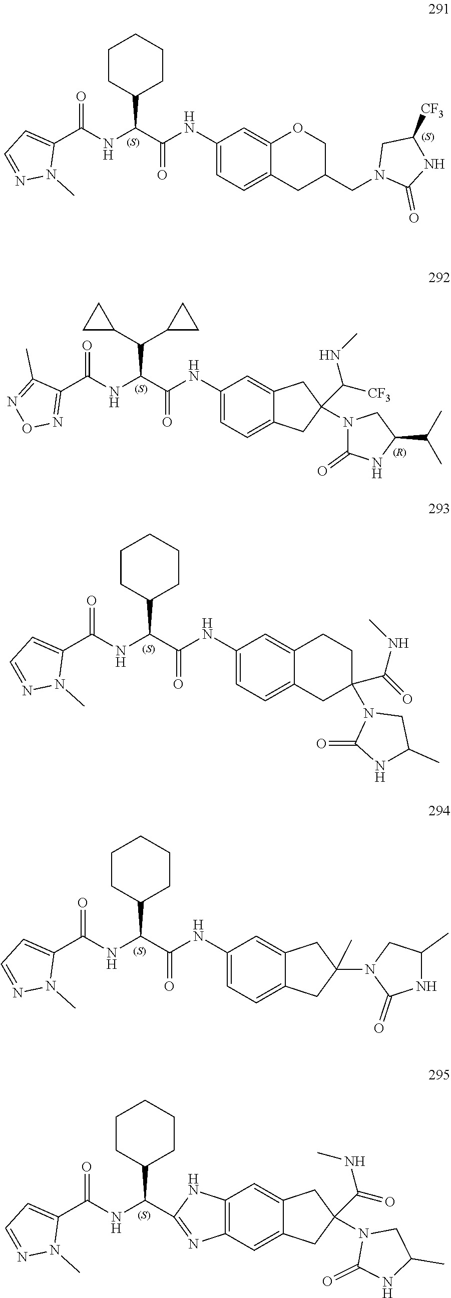

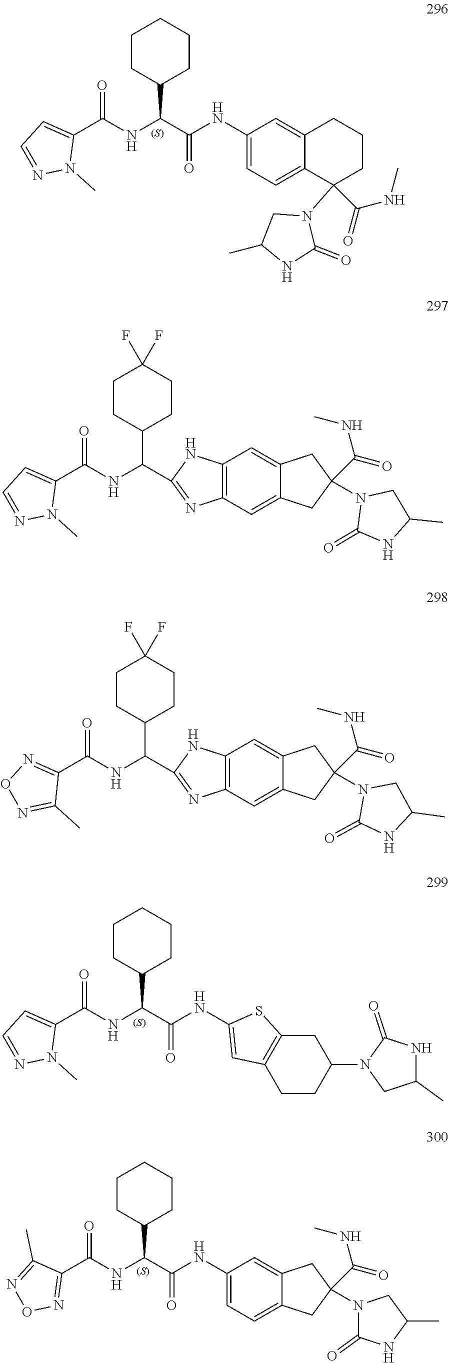

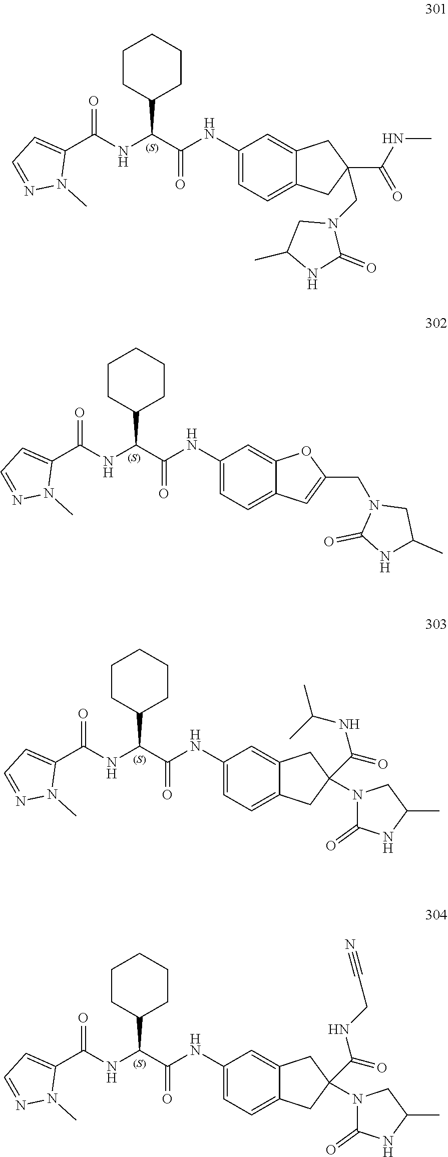

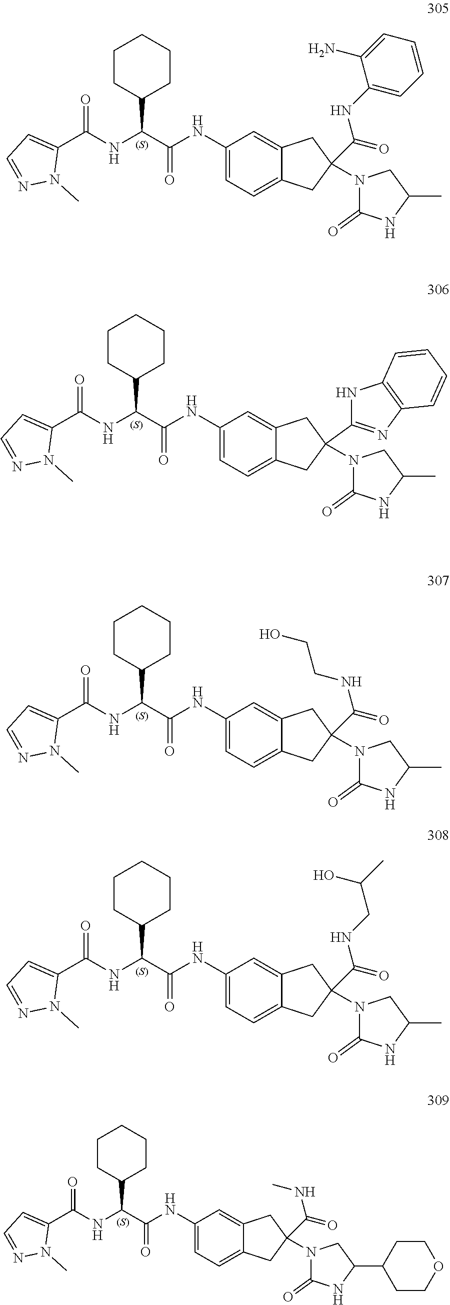

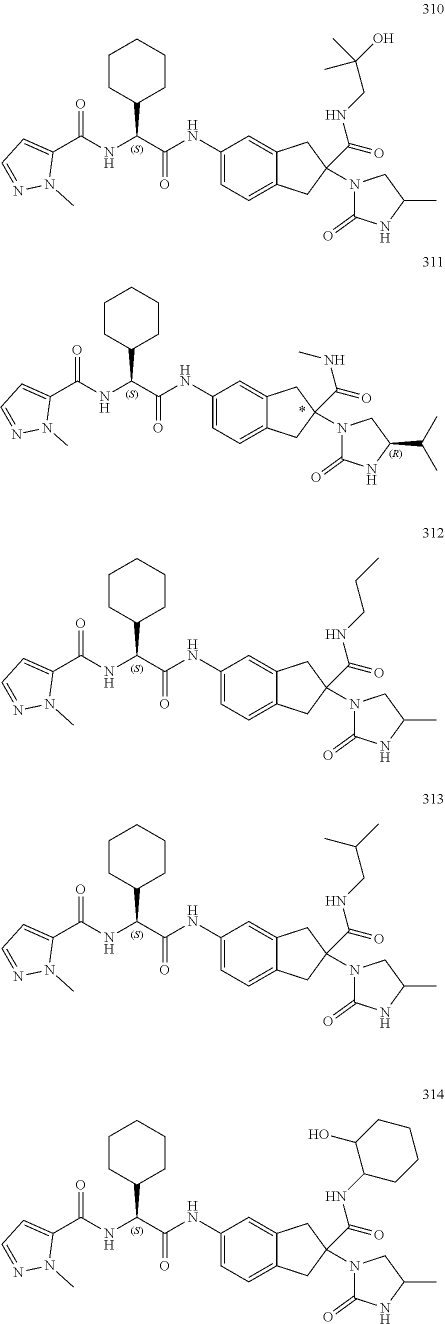

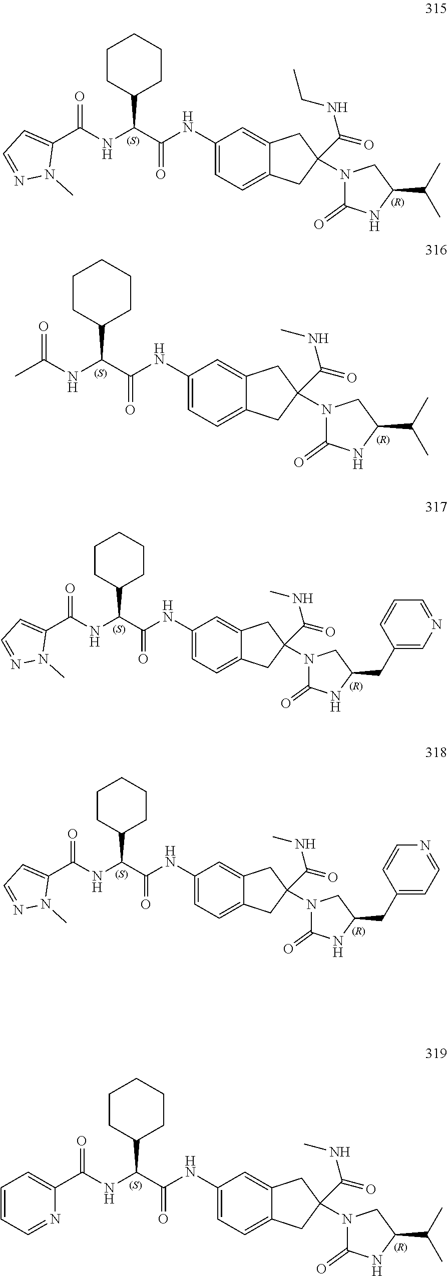

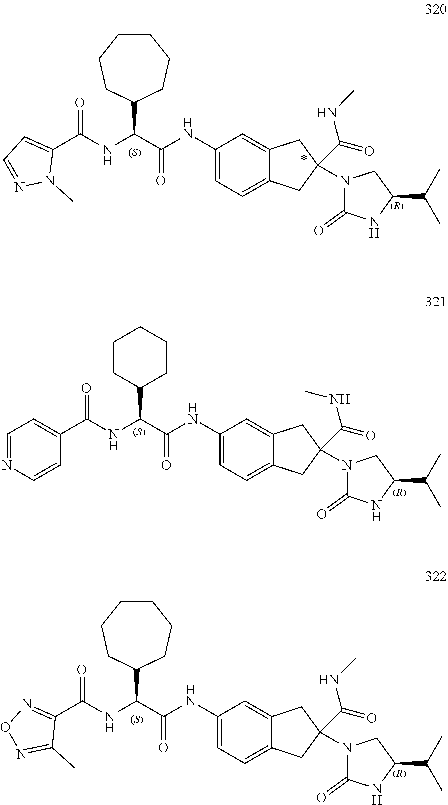

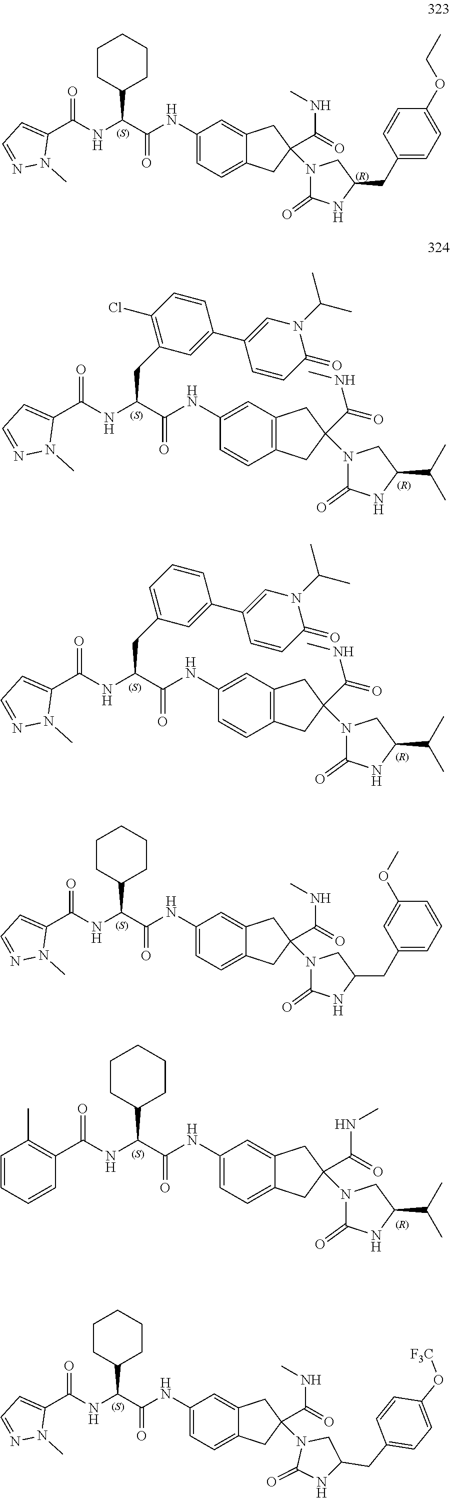

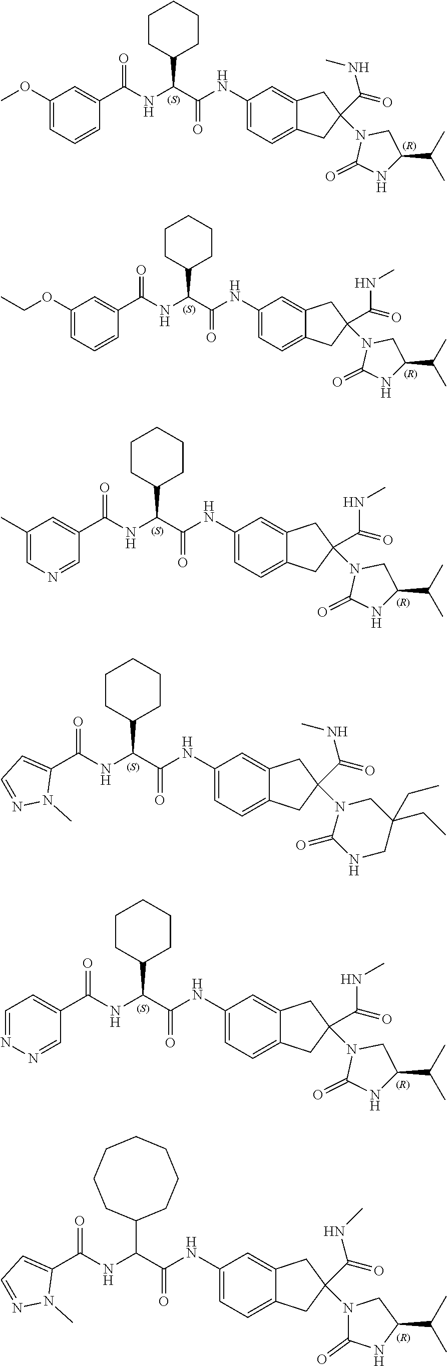

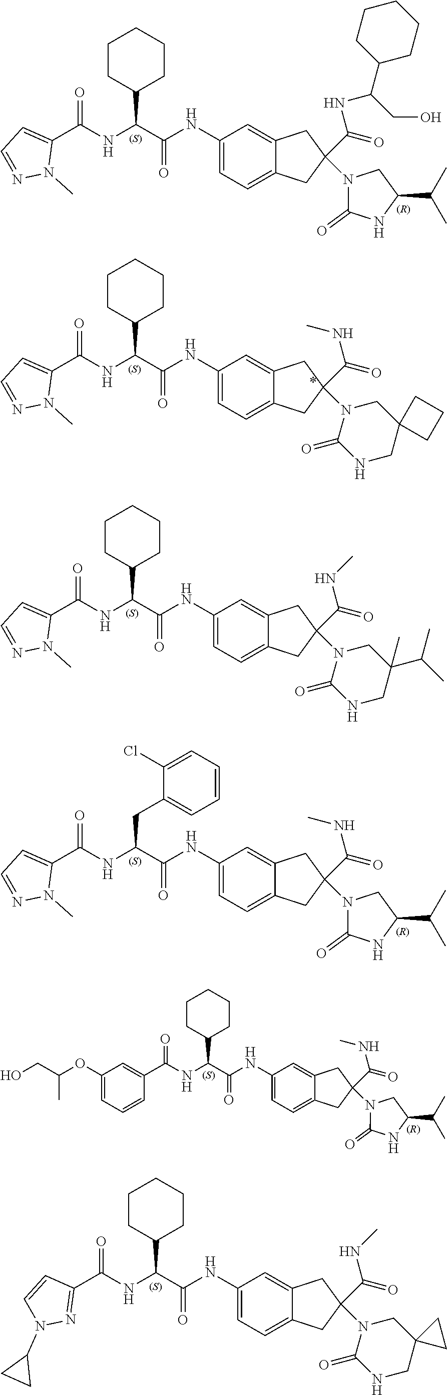

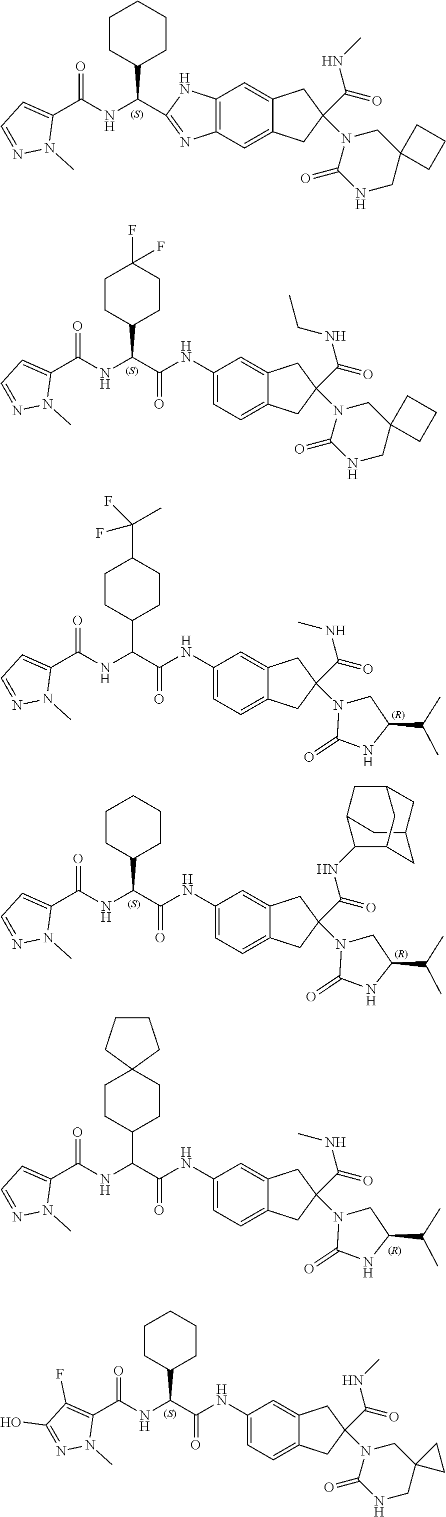

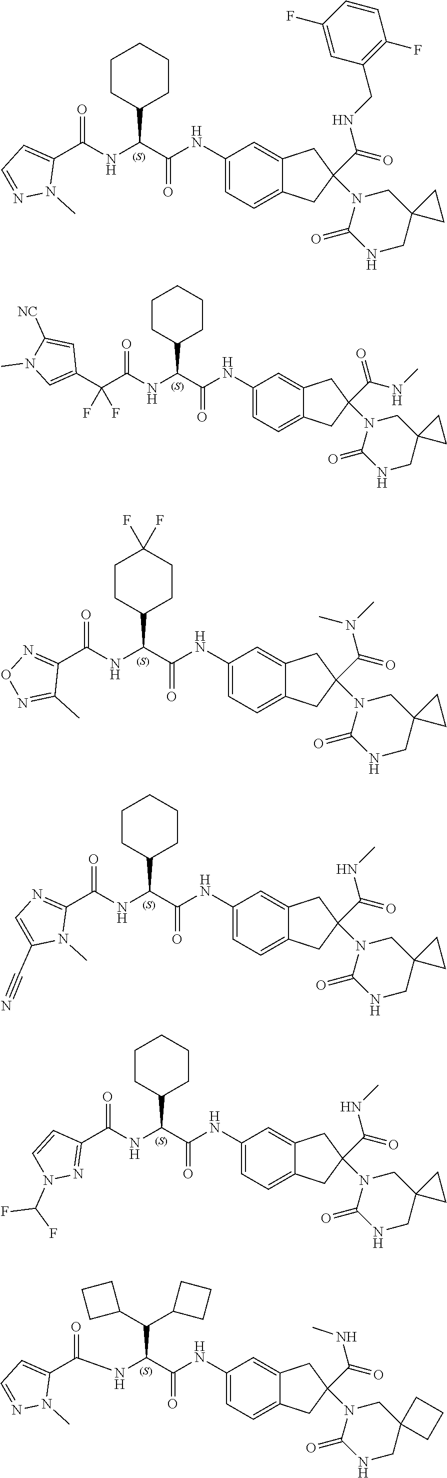

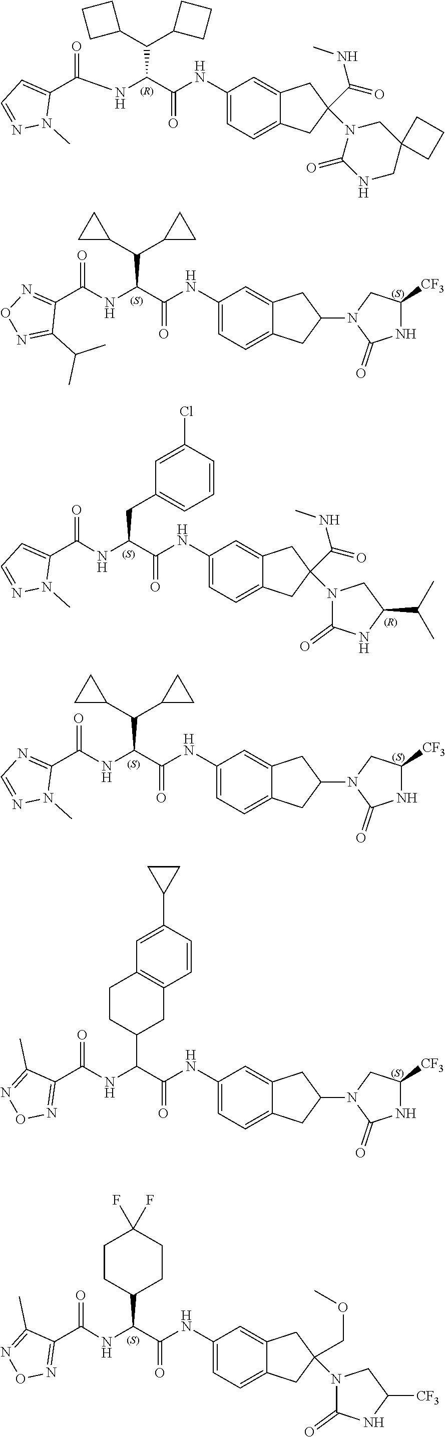

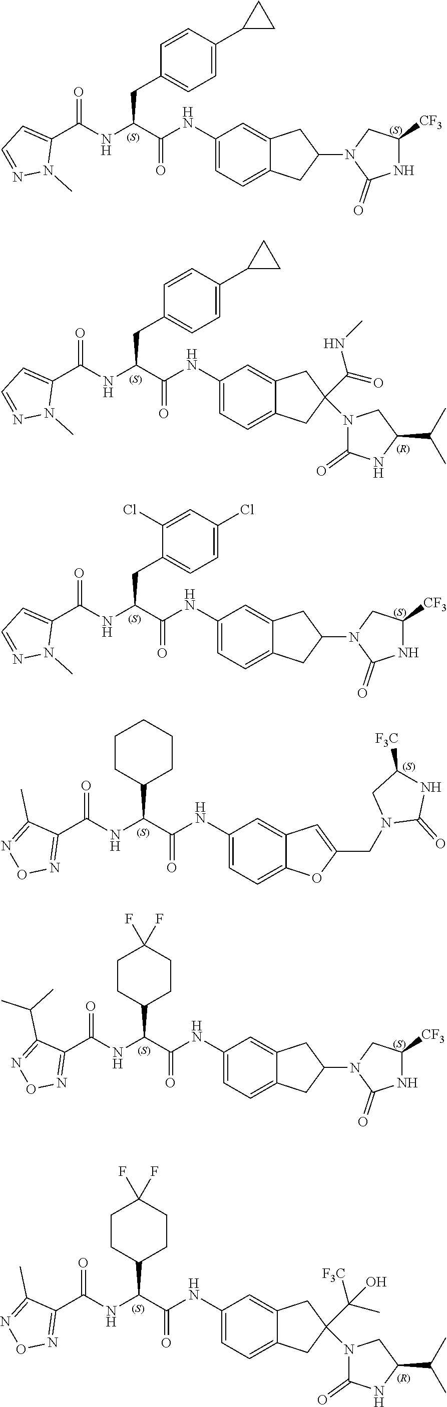

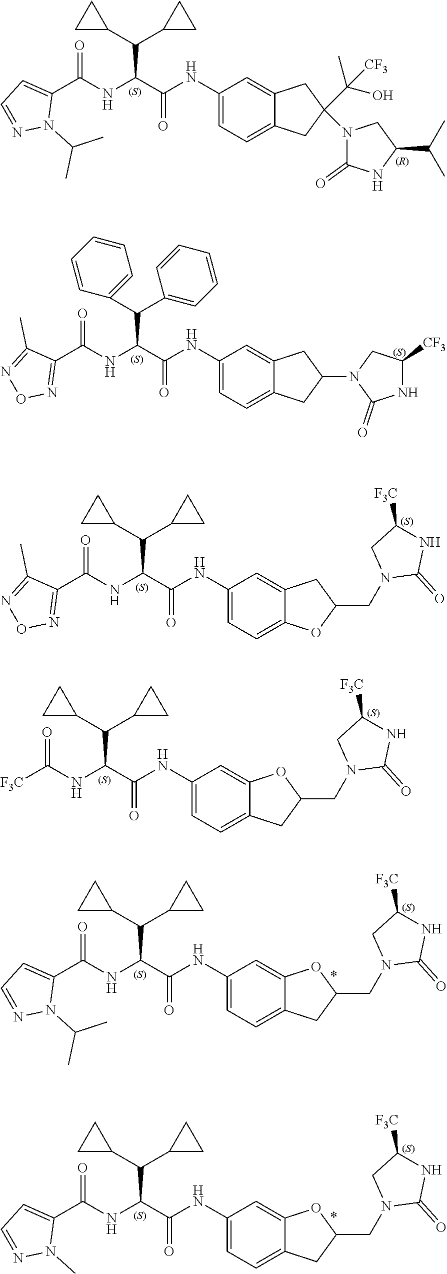

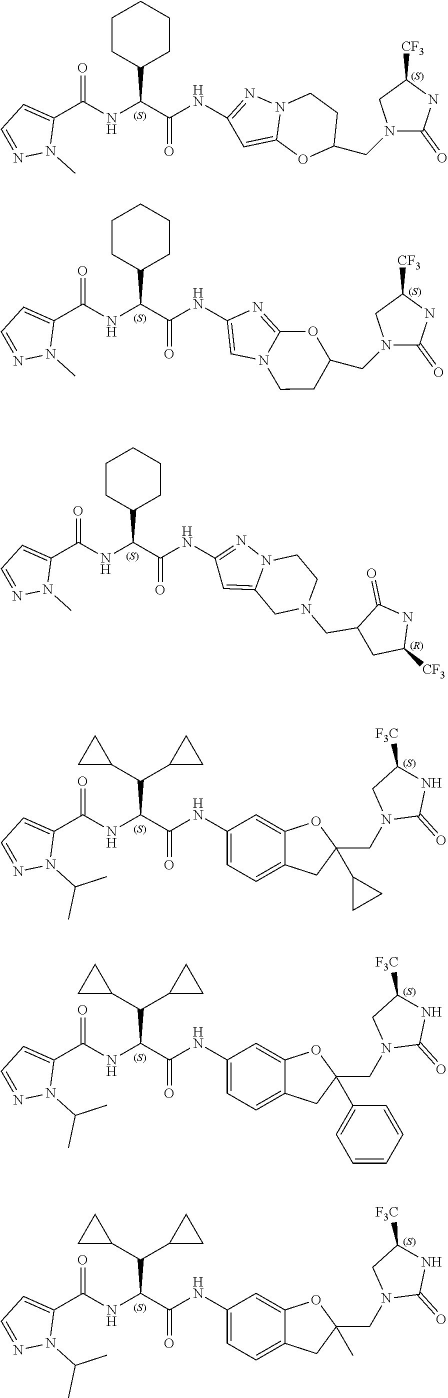

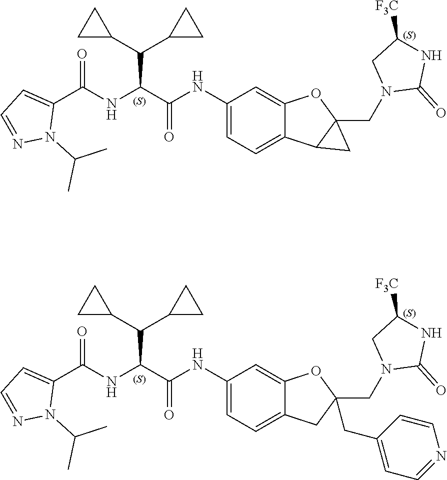

33. A pharmaceutical composition comprising a compound of any one of claims 1-32 or a pharmaceutically acceptable salt, stereoisomer, tautomer or prodrug thereof, together with a
pharmaceutically acceptable excipient.

34. A method of decreasing IL-17 activity by inhibition, which comprises administering to an individual the compound according to any one of claims 1-32, or a pharmaceutically acceptable salt thereof, including the compound of formula (I) or the specific compounds exemplified herein.

35. The method of claim 34, wherein the disease is selected from cancer.

36. Use of a compound of any one of claims 1-32 or a pharmaceutically acceptable salt, stereoisomer, tautomer or prodrug thereof in the preparation of a medicament for treating a disease that can be affected by IL-17.

37. The use of claim 36, wherein the disease is autoimmune disease or inflammatory disease.

38. The use of claim 37, wherein the disease is chronic inflammation, psoriasis, spondylarthritis, rheumatoid arthritis or multiple sclerosis.
